(19) United States
(12) Patent Application Publication
(10) Pub. No.: US 2024/0327415 A1
(43) Pub. Date: Oct. 3, 2024

(54) Multicomponent Complexes, Including Salts, Crystalline Forms, and Hydrates and Solvates of ENT1 Inhibitors

(71) Applicant: iTeos Belgium SA, Gosselies (BE)

(72) Inventors: Ron Lawrence, Bedfordshire (GB); Hayley Ann Reece, Dalkeith (GB); Juliet Adeoye, Bonnyrigg (GB); Laura Allan, Dalkeith (GB); Erica Joke Katelijne Heleen Houthuys, Halle (BE)

(73) Assignee: iTeos Belgium SA, Gosselies (BE)

(21) Appl. No.: 18/612,120

(22) Filed: Mar. 21, 2024

Related U.S. Application Data
(60) Provisional application No. 63/491,684, filed on Mar. 22, 2023.
(60) Provisional application No. 63/494,361, filed on Apr. 5, 2023.

(51) Int. Cl. C07D 487/04; A61K 31/551; A61K 45/06

(52) U.S. Cl. CPC C07D 487/04; A61K 31/551; A61K 45/06

(57) ABSTRACT

In some embodiments, the present disclosure relates to multicomponent complexes, such as salts, of Compound 1, and hydrates and solvates thereof. In some embodiments, the present disclosure relates to pharmaceutically acceptable salts of Compound 1, and hydrates and solvates thereof. In some embodiments, the present disclosure relates to crystalline complexes comprising Compound 1 (free base) and a coformer, and hydrates and solvates thereof. In some embodiments, the present disclosure relates to a crystalline salt of Compound 1 comprising Compound 1 (free base) and a salt coformer, and hydrates and solvates thereof. In some embodiments, the present disclosure relates to crystalline forms of Compound 1 (free base), and hydrates and solvates thereof. In some embodiments, the present disclosure relates to compositions, dosage forms, and kits comprising a multicomponent complex as disclosed herein. In some embodiments, the present disclosure relates to methods of inhibiting ENT1 and methods of treating cancer comprising administering a multicomponent complex as disclosed herein.

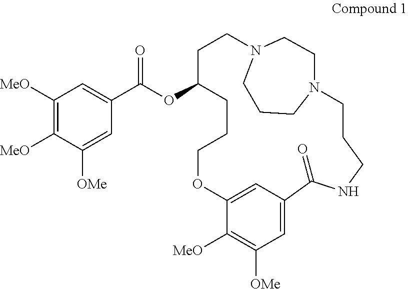 [SMILES: COc1cc(C(=O)O[C@@H]2CCCOc3cc(cc(OC)c3OC)C(=O)NCCCN3CCCN(CC2)CC3)cc(OC)c1OC]

### FIELD

In some embodiments, the present disclosure relates to multicomponent complexes, such as salts, of Compound 1, and hydrates and solvates thereof. In some embodiments, the present disclosure relates to pharmaceutically acceptable salts of Compound 1, and hydrates and solvates thereof. In some embodiments, the present disclosure relates to crystalline complexes comprising Compound 1 (free base) and a coformer, and hydrates and solvates thereof. In some embodiments, the present disclosure relates to a crystalline salt of Compound 1 comprising Compound 1 (free base) and a salt coformer, and hydrates and solvates thereof. In some embodiments, the present disclosure relates to crystalline forms of Compound 1 (free base), and hydrates and solvates thereof.

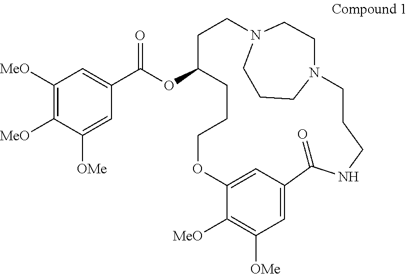 [SMILES: COc1cc(C(=O)O[C@@H]2CCCOc3cc(cc(OC)c3OC)C(=O)NCCCN3CCCN(CC2)CC3)cc(OC)c1OC]

### Background and Summary

The equilibrative nucleoside transporter (ENT) family, also known as SLC29, is a group of plasmalemmal transport proteins which transport nucleoside substrates into cells. There are four known ENTs, designated ENT1, ENT2, ENT3, and ENT4.

One of the endogenous substrates for ENTs is adenosine, a potent physiological and pharmacological regulator of numerous functions. Cellular signaling by adenosine occurs through four known G-protein-coupled adenosine receptors A1, A2A, A2B, and A3. By influencing the concentration of adenosine available to these receptors, ENTs fulfill important regulatory roles in different physiological processes, such as modulation of coronary blood flow, inflammation, and neurotransmission (Griffith D A and Jarvis S M, Biochim Biophys Acta, 1996, 1286, 153-181; Shryock J C and Belardinelli L, Am J Cardiol, 1997, 79(12A), 2-10; Anderson C M et al., J Neurochem, 1999, 73, 867-873).

Adenosine is also a potent immunosuppressive metabolite that is often found elevated in the extracellular tumor microenvironment (TME) (Blay J et al., Cancer Res, 1997, 57, 2602-2605). Extracellular adenosine is generated mainly by the conversion of ATP by the ectonucleotidases CD39 and CD73 (Stagg J and Smyth M J, Oncogene, 2010, 2, 5346-5358). Adenosine activates four G-protein-coupled receptor subtypes (A1, A2A, A2B, and A3). In particular, activation of the A2A receptor is believed to be the main driver of innate and adaptive immune cell suppression leading to suppression of antitumor immune responses (Ohta and Sitkovsky, Nature, 2001, 414, 916-920; Stagg and Smyth, Oncogene, 2010, 2, 5346-5358; Antonioli L et al., Nature Reviews Cancer, 2013, 13, 842-857; Cekic C and Linden J, Nature Reviews, Immunology, 2016, 16, 177-192; Allard B et al., Curr Op Pharmacol, 2016, 29, 7-16; Vijayan D et al., Nature Reviews Cancer, 2017, 17, 709-724).

The Applicant previously evidenced in PCT/EP2019/076244 that adenosine as well as ATP profoundly suppress T cell proliferation and cytokine secretion (IL-2), and strongly reduce T cell viability. Adenosine- and ATP-mediated suppression of T cell viability and proliferation were successfully restored by using ENTs inhibitors. Moreover, the use of an ENT inhibitor in combination with an adenosine receptor antagonist enabled to restore not only adenosine- and ATP-mediated suppression of T cell viability and proliferation, but also restored T cell cytokine secretion. These results showed that ENTs inhibitors either alone or in combination with an adenosine receptor antagonist may be useful for the treatment of cancers.

A variety of drugs such as dilazep, dipyridamole, and draflazine interact with ENTs and alter adenosine levels, and were developed for their cardioprotective or vasodilatory effects.

Currently, two non-selective ENT1 inhibitors (dilazep and dipyridamole) are on the market (Vlachodimou et ah, Bio-Chemical Pharmacology, 2020, 172, 113747). However, their binding kinetics are unknown.

Novel macrocyclic diamine selective ENT inhibitors have recently been disclosed. See, e.g., WO 2021/204896, the entire content of which is incorporated herein by reference. For example, (12R)-7⁴,7⁵-dimethoxy-6-oxo-8-oxa-5-aza-1(1,4)-diazepana-7(1,3)-benzenacyclotetradecaphane-12-yl 3,4,5-trimethoxybenzoate (Compound 10 in WO 2021/204896) has been shown to effectively inhibit ENT1. See id. at p. 197 (showing Compound 10 exhibits an IC50 between 0.001 and 0.02 μM in a human ENT1 binding assay). However, that compound (called “Compound 1” or “Compound 1 (free base)” herein and with the formula shown below)

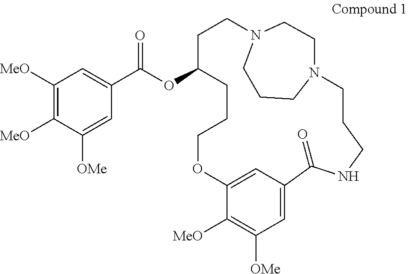 [SMILES: COc1cc(C(=O)O[C@@H]2CCCOc3cc(cc(OC)c3OC)C(=O)NCCCN3CCCN(CC2)CC3)cc(OC)c1OC]

as prepared in WO 2021/204896 is not a salt and was also not prepared as a crystalline solid. Salts are often more desirable than free bases or free acids for drug delivery and manufacturing, and amorphous forms are generally more unstable than crystalline forms. Crystalline solids tend to be more favorable for processing, storage, and stability than non-crystalline solids. Crystalline solids tend to be more favorable for processing, storage, and stability than non-crystalline solids.

Indeed, amorphous Compound 1 was found to be hygroscopic and have a propensity to absorb at least moderate amounts of water. As such, there is a need to create alternative crystalline and/or salt forms of Compound 1. However, energetics may not favor the ready formation of suitable crystalline solids and it is unpredictable what, if any crystalline solids will be suitable. Additionally, polymorphism may make creating stable crystalline solids of a particular active complex and difficult to predict.

In one aspect, the present disclosure relates to solid forms of Compound 1:

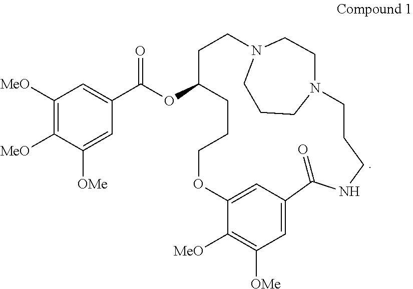 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to salts and crystalline complexes of Compound 1. In one aspect, the present disclosure relates to organic salts and crystalline complexes of Compound 1. In one aspect, the present disclosure relates to inorganic salts and crystalline complexes of Compound 1, wherein the salt or a hydrate or solvate thereof is not a hexafluorophosphate salt of Compound 1 or a hydrate or solvate thereof.

In one aspect, the present disclosure relates to a salt of Compound 1 and hydrates and solvates thereof, wherein the salt or a hydrate or solvate thereof is not a hexafluorophosphate salt of Compound 1 or a hydrate or solvate thereof. In some embodiments, the present disclosure relates to a salt of Compound 1 and hydrates and solvates thereof, wherein the salt or a hydrate or solvate thereof is not a salt formed from Compound 1 and benzotriazol-1-yloxytripyrrolidinophosphonium hexafluorophosphate (PyBOP).

In one aspect, the present disclosure relates to a pharmaceutically acceptable salt of Compound 1, and hydrates and solvates thereof.

In one aspect, the present disclosure relates to a crystalline complex comprising Compound 1 and a coformer, and hydrates and solvates of the crystalline complex.

In one aspect, the present disclosure relates to a crystalline salt of Compound 1 comprising Compound 1 and a salt coformer, and hydrates and solvates of the crystalline salt.

In one aspect, the present disclosure relates to Compound 1 hydrogen sulfate, and hydrates and solvates thereof.

In one aspect, the present disclosure relates to crystalline Form 1 Compound 1 di(hydrogen sulfate), and hydrates and solvates thereof. In one aspect, the present disclosure relates to crystalline Form 2 Compound 1 di(hydrogen sulfate) trihydrate. In one aspect, the present disclosure relates to crystalline Form 3 Compound 1 di(hydrogen sulfate), and hydrates and solvates thereof. In one aspect, the present disclosure relates to crystalline Form 5 Compound 1 di(hydrogen sulfate), and hydrates and solvates thereof. In one aspect, the present disclosure relates to crystalline Form 6 Compound 1 di(hydrogen sulfate), and hydrates and solvates thereof. In one aspect, the present disclosure relates to crystalline Form 7 Compound 1 di(hydrogen sulfate), and hydrates and solvates thereof. In one aspect, the present disclosure relates to crystalline Form 9 Compound 1 di(hydrogen sulfate), and hydrates and solvates thereof. In one aspect, the present disclosure relates to amorphous Compound 1 di(hydrogen sulfate), and hydrates and solvates thereof.

In one aspect, the present disclosure relates to crystalline Form 4 Compound 1 mono(hydrogen sulfate), and hydrates and solvates thereof. In one aspect, the present disclosure relates to crystalline Form 8 Compound 1 mono(hydrogen sulfate), and hydrates and solvates thereof. In one aspect, the present disclosure relates to crystalline Form 10 Compound 1 mono(hydrogen sulfate), and hydrates and solvates thereof. In one aspect, the present disclosure relates to amorphous Compound 1 mono(hydrogen sulfate), and hydrates and solvates thereof.

In one aspect, the present disclosure relates to amorphous Compound 1 hydrogen chloride and hydrates and solvates thereof. In one aspect, the present disclosure relates to amorphous Compound 1 mono(hydrogen chloride) and hydrates and solvates thereof. In one aspect, the present disclosure relates to amorphous Compound 1 di(hydrogen chloride) and hydrates and solvates thereof.

In one aspect, the present disclosure relates to crystalline Compound 1 hydrogen chloride and hydrates and solvates thereof. In one aspect, the present disclosure relates to crystalline Compound 1 di(hydrogen chloride) and hydrates and solvates thereof. In one aspect, the present disclosure relates to crystalline Form 1 Compound 1 hydrogen chloride and hydrates and solvates thereof.

In one aspect, the present disclosure relates to crystalline Compound 1 hydrogen phosphate and hydrates and solvates thereof. In one aspect, the present disclosure relates to crystalline Compound 1 mono(hydrogen phosphate) and hydrates and solvates thereof. In one aspect, the present disclosure relates to crystalline Compound 1 di(hydrogen phosphate) and hydrates and solvates thereof.

In one aspect, the present disclosure relates to crystalline Form 1 Compound 1 hydrogen phosphate and hydrates and solvates thereof. In one aspect, the present disclosure relates to crystalline Form 2 Compound 1 hydrogen phosphate and hydrates and solvates thereof. In one aspect, the present disclosure relates to crystalline Form 3 Compound 1 hydrogen phosphate and hydrates and solvates thereof. In one aspect, the present disclosure relates to Crystalline Form 4 Compound 1 hydrogen phosphate and hydrates and solvates thereof.

In one aspect, the present disclosure relates to crystalline Form 5 Compound 1 hydrogen phosphate and hydrates and solvates thereof. In one aspect, the present disclosure relates to crystalline Form 6 Compound 1 hydrogen phosphate and hydrates and solvates thereof. In one aspect, the present disclosure relates to crystalline Form 7 Compound 1 hydrogen phosphate and hydrates and solvates thereof. In one aspect, the present disclosure relates to crystalline Form 8 Compound 1 hydrogen phosphate and hydrates and solvates thereof.

In one aspect, the present disclosure relates to an amorphous salt of Compound 1 comprising Compound 1 and a salt coformer, and hydrates and solvates of said amorphous salt. In one aspect, the present disclosure relates to an amorphous salt of Compound 1 comprising Compound 1 and a salt coformer, and hydrates and solvates of said amorphous salt, wherein the amorphous salt or a hydrate or solvate thereof is not a hexafluorophosphate salt of Compound 1 or a hydrate or solvate thereof.

In one aspect, the present disclosure relates to crystalline Compound 1 fumarate and hydrates and solvates thereof. In one aspect, the present disclosure relates to crystalline Compound 1 mono(fumarate) and hydrates and solvates thereof. In one aspect, the present disclosure relates to crystalline Compound 1 di(fumarate) and hydrates and solvates thereof.

In one aspect, the present disclosure relates to crystalline Form 1 Compound 1 fumarate and hydrates and solvates thereof. In one aspect, the present disclosure relates to crystalline Form 2 Compound 1 fumarate and hydrates and solvates thereof. In one aspect, the present disclosure relates to crystalline Form 3 Compound 1 fumarate and hydrates and solvates thereof.

In one aspect, the present disclosure relates to crystalline Compound 1 L-malate and hydrates and solvates thereof. In one aspect, the present disclosure relates to crystalline Compound 1 mono(L-malate) and hydrates and solvates thereof. In one aspect, the present disclosure relates to crystalline Compound 1 di(L-malate) and hydrates and solvates thereof.

In one aspect, the present disclosure relates to crystalline Compound 1 benzoate and hydrates and solvates thereof. In one aspect, the present disclosure relates to crystalline Compound 1 mono(benzoate) and hydrates and solvates thereof. In one aspect, the present disclosure relates to crystalline Compound 1 di(benzoate) and hydrates and solvates thereof.

In one aspect, the present disclosure relates to crystalline Form 1 Compound 1 benzoate and hydrates and solvates thereof. In one aspect, the present disclosure relates to crystalline Form 2 Compound 1 benzoate and hydrates and solvates thereof. In one aspect, the present disclosure relates to crystalline Form 3 Compound 1 benzoate and hydrates and solvates thereof.

In one aspect, the present disclosure relates to a substantially crystalline salt of Compound 1, and hydrates and solvates thereof. In one aspect, the present disclosure relates to a substantially crystalline salt of Compound 1, and hydrates and solvates thereof, wherein the salt or a hydrate or solvate thereof is not a hexafluorophosphate salt of Compound 1. In some embodiments, the present disclosure relates a substantially crystalline salt of Compound 1, and hydrates and solvates thereof, wherein the salt or a hydrate or solvate thereof is not a salt formed from Compound 1 and benzotriazol-1-yloxytripyrrolidinophosphonium hexafluorophosphate (PyBOP).

In one aspect, the present disclosure relates to a salt selected from Compound 1 hydrogen chloride, Compound 1 hydrogen sulfate, Compound 1 hydrogen phosphate, Compound 1 tosylate, Compound 1 mesylate, Compound 1 benzenesulfonate, Compound 1 L-tartrate, Compound 1 fumarate, Compound 1 citrate, Compound 1 maleate, Compound 1 L-malate, Compound 1 benzoate, and hydrates and solvates thereof.

In one aspect, the present disclosure relates to crystalline Compound 1, and hydrates and solvates thereof.

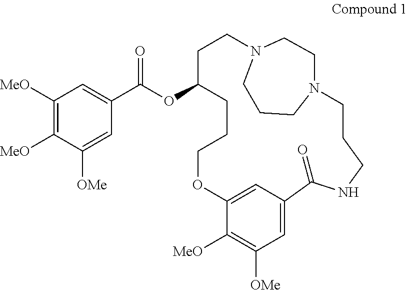 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to crystalline Form 1 Compound 1, and hydrates and solvates thereof. In one aspect, the present disclosure relates to crystalline Form 2 Compound 1, and hydrates and solvates thereof.

In one aspect, the present disclosure relates to a substantially crystalline salt of Compound 1 and hydrates and solvates thereof.

In one aspect, the present disclosure relates to substantially crystalline Compound 1 di(hydrogen sulfate), and hydrates and solvates thereof. In one aspect, the present disclosure relates to substantially crystalline Form 2 Compound 1 di(hydrogen sulfate) trihydrate.

In one aspect, the present disclosure relates to a composition comprising one or more salts of Compound 1 or hydrates or solvates thereof.

In one aspect, the present disclosure relates to a composition comprising two or more of crystalline Form 1 Compound 1 di(hydrogen sulfate), crystalline Form 2 Compound 1 di(hydrogen sulfate) trihydrate, crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), and hydrates and solvates thereof.

In one aspect, the present disclosure relates to a composition comprising substantially crystalline Form 2 Compound 1 di(hydrogen sulfate) trihydrate further comprising one or more of crystalline Form 1 Compound 1 di(hydrogen sulfate), crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), and hydrates and solvates thereof.

In one aspect, the present disclosure relates to a composition comprising amorphous Compound 1 di(hydrogen sulfate) or a hydrate or solvate thereof, and one or more of crystalline Form 1 Compound 1 di(hydrogen sulfate), crystalline Form 2 Compound 1 di(hydrogen sulfate) trihydrate, crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), and hydrates and solvates thereof.

In one aspect, the present disclosure relates to a composition comprising amorphous Compound 1 mono(hydrogen sulfate) or a hydrate or solvate thereof, and one or more of crystalline Form 4 Compound 1 mono(hydrogen sulfate), crystalline Form 8 Compound 1 mono(hydrogen sulfate), crystalline Form 10 Compound 1 mono(hydrogen sulfate), and hydrates and solvates thereof.

In one aspect, the present disclosure relates to a pharmaceutical composition comprising a salt of Compound 1 or a hydrate or solvate thereof, and at least one pharmaceutically acceptable excipient. In some embodiments, the salt is selected from Compound 1 hydrogen chloride, Compound 1 hydrogen sulfate, Compound 1 hydrogen phosphate, Compound 1 tosylate, Compound 1 mesylate, Compound 1 benzenesulfonate, Compound 1 L-tartrate, Compound 1 fumarate, Compound 1 citrate, Compound 1 maleate, Compound 1 L-malate, Compound 1 benzoate, and hydrates and solvates thereof.

In one aspect, the present disclosure relates to a pharmaceutical composition comprising: a Compound 1 hydrogen sulfate or a hydrate or solvate thereof or a composition as disclosed herein, and at least one pharmaceutically acceptable excipient.

In one aspect, the present disclosure relates to a pharmaceutical composition comprising crystalline Compound 1 or a hydrate or solvate thereof and at least one pharmaceutically acceptable excipient.

In one aspect, the present disclosure relates to a dosage form comprising a composition as disclosed herein.

In one aspect, the present disclosure relates to a method of inhibiting ENT1 in a patient need thereof, comprising: administering to said patient an effective amount of a salt of Compound 1 or a hydrate or solvate thereof; an effective amount of crystalline Compound 1 or a hydrate or solvate thereof; an effective amount of a composition as disclosed herein; or a dosage form as disclosed herein. In one aspect, the present disclosure relates to a method of inhibiting ENT1 in a patient need thereof, comprising: administering to said patient an effective amount of a Compound 1 hydrogen sulfate or a hydrate or solvate thereof.

In one aspect, the present disclosure relates to use of a salt of Compound 1 or a hydrate or solvate thereof; an effective amount of crystalline Compound 1 or a hydrate or solvate thereof; a composition as disclosed herein; or a dosage form as disclosed herein, for the manufacture of a medicament for inhibiting ENT1. In one aspect, the present disclosure relates to a use of an effective amount of a Compound 1 hydrogen sulfate or a hydrate or solvate thereof, for the manufacture of a medicament for inhibiting ENT1.

In one aspect, the present disclosure relates to a salt of Compound 1 or a hydrate or solvate thereof; a composition as described herein; or a dosage form as described herein for use as a medicament.

In one aspect, the present disclosure relates to a method of treating cancer in a patient need thereof, comprising: administering to said patient an effective amount of a salt of Compound 1 or a hydrate or solvate thereof; an effective amount of crystalline Compound 1 or a hydrate or solvate thereof; an effective amount of a composition as disclosed herein; or a dosage form as disclosed herein. In one aspect, the present disclosure relates to a method of treating cancer in a patient need thereof, comprising: administering to said patient an effective amount of a Compound 1 hydrogen sulfate or a hydrate or solvate thereof.

In one aspect, the present disclosure relates to a use of a salt of Compound 1 or a hydrate or solvate thereof; a crystalline Compound 1 or a hydrate or solvate thereof; a composition as disclosed herein; or a dosage form as disclosed herein, for the manufacture of a medicament for treating cancer. In one aspect, the present disclosure relates to a use of a Compound 1 hydrogen sulfate or a hydrate or solvate thereof, for the manufacture of a medicament for treating cancer.

In one aspect, the present disclosure relates to a salt of Compound 1 or a hydrate or solvate thereof; a crystalline Compound 1 or a hydrate or solvate thereof, a composition as disclosed herein; or a dosage form as disclosed herein, for use in a method of treating cancer.

In one aspect, the present disclosure relates to a method of treating cancer in a patient need thereof, comprising: administering to said patient (1) an effective amount of a salt of Compound 1 or a hydrate or solvate thereof; an effective amount of crystalline Compound 1 or a hydrate or solvate thereof; an effective amount of a composition as disclosed herein; or a dosage form as disclosed herein; and (2) an adenosine receptor antagonist. In one aspect, the present disclosure relates to a method of treating cancer in a patient need thereof, comprising: administering to said patient (1) an effective amount of Compound 1 hydrogen sulfate or a hydrate or solvate thereof; and (2) an adenosine receptor antagonist.

In one aspect, the present disclosure relates to a use of a combination of (1) a salt of Compound 1 or a hydrate or solvate thereof; an effective amount of crystalline Compound 1 or a hydrate or solvate thereof; a composition as disclosed herein; or a dosage form as disclosed herein; and (2) an adenosine receptor antagonist for the manufacture of a medicament for treating cancer. In one aspect, the present disclosure relates to a use of a combination of (1) an effective amount of Compound 1 hydrogen sulfate or a hydrate or solvate thereof; and (2) an adenosine receptor antagonist for the manufacture of a medicament for treating cancer.

In one aspect, the present disclosure relates to a process for preparing Compound 1 di(hydrogen sulfate) or a hydrate or solvate thereof comprising the steps of dissolving Compound 1 (free base) or a hydrate or solvate thereof in a suitable solvent to form a solution, treating the solution with sulfuric acid, and removing the solvent from the solution to form the Compound 1 di(hydrogen sulfate) or a hydrate or solvate thereof.

In one aspect, the present disclosure relates to Compound 1 di(hydrogen sulfate) or a hydrate or solvate thereof prepared by a process comprising the steps of dissolving Compound 1 (free base) or a hydrate or solvate thereof in a suitable solvent to form a solution, treating the solution with sulfuric acid, and removing the solvent from the solution to form the Compound 1 di(hydrogen sulfate) or a hydrate or solvate thereof.

In one aspect, the present disclosure relates to a process for preparing Compound 1 mono(hydrogen sulfate) or a hydrate or solvate thereof comprising the steps of dissolving a Compound 1 di(hydrogen sulfate) or a hydrate or solvate thereof in a suitable solvent to form a solution and removing the solvent from the solution to form the Compound 1 mono(hydrogen sulfate) or a hydrate or solvate thereof.

In one aspect, the present disclosure relates to Compound 1 mono(hydrogen sulfate) or a hydrate or solvate thereof prepared by a process comprising the steps of dissolving a Compound 1 di(hydrogen sulfate) or a hydrate or solvate thereof in a suitable solvent to form a solution and removing the solvent from the solution to form the Compound 1 mono(hydrogen sulfate) or a hydrate or solvate thereof.

### BRIEF DESCRIPTION OF THE DRAWINGS

FIG. 1 shows an XRPD pattern of crystalline Form 1 Compound 1 di(hydrogen sulfate).

FIG. 2 shows a peak picked XRPD pattern of crystalline Form 1 Compound 1 di(hydrogen sulfate).

FIG. 3 shows an XRPD pattern of crystalline Form 2 Compound 1 di(hydrogen sulfate) trihydrate.

FIG. 4 shows a peak picked XRPD pattern of crystalline Form 2 Compound 1 di(hydrogen sulfate) trihydrate.

FIG. 5 shows an XRPD pattern of crystalline Form 3 Compound 1 di(hydrogen sulfate).

FIG. 6 shows a peak picked XRPD pattern of crystalline Form 3 Compound 1 di(hydrogen sulfate).

FIG. 7 shows an XRPD pattern of damp crystalline Form 4 Compound 1 mono(hydrogen sulfate).

FIG. 8 shows a peak picked XRPD pattern of damp crystalline Form 4 Compound 1 mono(hydrogen sulfate).

FIG. 9 shows an XRPD pattern of dry crystalline Form 4 Compound 1 mono(hydrogen sulfate).

FIG. 10 shows a peak picked XRPD pattern of dry crystalline Form 4 Compound 1 mono(hydrogen sulfate).

FIG. 11 shows an XRPD pattern of crystalline Form 5 Compound 1 di(hydrogen sulfate).

FIG. 12 shows a peak picked XRPD pattern of crystalline Form 5 Compound 1 di(hydrogen sulfate).

FIG. 13 shows an XRPD pattern of crystalline Form 6 Compound 1 di(hydrogen sulfate).

FIG. 14 shows a peak picked XRPD pattern of crystalline Form 6 Compound 1 di(hydrogen sulfate).

FIG. 15 shows an XRPD pattern of crystalline Form 7 Compound 1 di(hydrogen sulfate).

FIG. 16 shows a peak picked XRPD pattern of crystalline Form 7 Compound 1 di(hydrogen sulfate).

FIG. 17 shows an XRPD pattern of crystalline Form 8 Compound 1 mono(hydrogen sulfate).

FIG. 18 shows a peak picked XRPD pattern of crystalline Form 8 Compound 1 mono(hydrogen sulfate).

FIG. 19 shows an XRPD pattern of crystalline Form 9 Compound 1 di(hydrogen sulfate).

FIG. 20 shows a peak picked XRPD pattern of crystalline Form 9 Compound 1 di(hydrogen sulfate).

FIG. 21 shows TG/DSC results of crystalline Form 3 Compound 1 di(hydrogen sulfate).

FIG. 22 shows DSC results of crystalline Form 3 Compound 1 di(hydrogen sulfate).

FIG. 23A shows an IR spectrum of crystalline Form 2 Compound 1 di(hydrogen sulfate) trihydrate.

FIG. 23B shows an IR spectrum of crystalline Form 3 Compound 1 di(hydrogen sulfate) prepared in Example 2A.

FIG. 24 shows a solution ¹H-NMR spectrum of dissolved crystalline Form 3 Compound 1 di(hydrogen sulfate) prepared in Example 2A, compared to an exemplary spectrum of dissolved crystalline Form 2 Compound 1 di(hydrogen sulfate) trihydrate.

FIG. 25 shows a DVS isotherm and kinetic plot of crystalline Form 3 Compound 1 di(hydrogen sulfate) prepared in Example 2A.

FIG. 26 shows the XRPD pattern of crystalline Form 3 Compound 1 di(hydrogen sulfate) prepared in Example 2A and the XRPD pattern of the sample post-DVS, compared to exemplary pattern of crystalline Form 2 Compound 1 di(hydrogen sulfate) trihydrate and crystalline Form 3 Compound 1 di(hydrogen sulfate).

FIGS. 27A, 27B, and 27C show XRPD patterns of crystalline Form 3 Compound 1 di(hydrogen sulfate) prepared in Example 2A when exposed to 20% to 90% RH.

FIGS. 28A and 28B show XRPD patterns at different temperatures of crystalline Form 3 Compound 1 di(hydrogen sulfate) prepared in Example 2A.

FIG. 29 shows XRPD patterns of crystalline Form 3 Compound 1 di(hydrogen sulfate) prepared in Example 2A and crystalline Form 9 Compound 1 di(hydrogen sulfate) prepared from heating crystalline Form 3 Compound 1 di(hydrogen sulfate) to 140° C.

FIG. 30 shows XRPD patterns of crystalline Form 2 Compound 1 di(hydrogen sulfate) trihydrate as prepared in Example 3-damp and dried samples-compared to an exemplary pattern for crystalline Form 2 Compound 1 di(hydrogen sulfate) trihydrate.

FIG. 31 shows TG/DSC results of crystalline Form 2 Compound 1 di(hydrogen sulfate) trihydrate prepared in Example 3.

FIG. 32 shows a DSC thermogram of crystalline Form 2 Compound 1 di(hydrogen sulfate) trihydrate prepared in Example 3.

FIG. 33 shows a ¹H-NMR spectrum of dissolved crystalline Form 2 Compound 1 di(hydrogen sulfate) trihydrate prepared in Example 3, compared to an exemplary spectrum of dissolved Compound 1.

FIG. 34 shows a DVS isothermal plot for crystalline Form 2 Compound 1 di(hydrogen sulfate) trihydrate prepared in Example 3.

FIG. 35 shows a DVS kinetic plot for crystalline Form 2 Compound 1 di(hydrogen sulfate) trihydrate prepared in Example 3.

FIG. 36 shows an XRPD patterns of crystalline Form 2 Compound 1 di(hydrogen sulfate) trihydrate prepared according to Example 4, compared to an exemplary pattern of crystalline Form 2 Compound 1 di(hydrogen sulfate) trihydrate.

FIG. 37 shows a Raman Spectrum of a sample of crystalline Form 2 Compound 1 di(hydrogen sulfate) trihydrate prepared according to Example 4.

FIG. 38 shows XRPD patterns of crystalline Form 2 Compound 1 di(hydrogen sulfate) trihydrate prepared according to Example 5-damp, dried, and after moisture equilibration samples-compared to an exemplary XRPD pattern and an exemplary simulated single crystal XRPD of crystalline Form 2 Compound 1 di(hydrogen sulfate) trihydrate.

FIG. 39 shows PLM images of crystalline Form 2 Compound 1 di(hydrogen sulfate) trihydrate samples prepared according to Example 5-damp, dried, and after moisture equilibration.

FIG. 40 shows an overlay of Particle Size Distributions of crystalline Form 2 Compound 1 di(hydrogen sulfate) trihydrate prepared according to Example 5 at 4.33% and 6.07% water contents.

FIG. 41 shows TG/DSC data of crystalline Form 2 Compound 1 di(hydrogen sulfate) trihydrate prepared according to Example 5.

FIG. 42 shows a DSC thermogram of crystalline Form 2 Compound 1 di(hydrogen sulfate) trihydrate prepared according to Example 5.

FIG. 43 shows a ¹H-NMR spectrum of dissolved crystalline Form 2 Compound 1 di(hydrogen sulfate) trihydrate prepared according to Example 5.

FIG. 44 shows an FT-Infrared spectrum of crystalline Form 2 Compound 1 di(hydrogen sulfate) trihydrate prepared according to Example 5.

FIG. 45 shows an XRPD pattern of amorphous Compound 1 di(hydrogen sulfate) prepared according to Example 7.

FIGS. 46A and 46B show XRPD patterns resulting from the variable humidity study on crystalline Form 2 Compound 1 di(hydrogen sulfate) trihydrate described in Example 9.

FIG. 47 shows XRPD patterns of crystalline Form 2 Compound 1 di(hydrogen sulfate) trihydrate input sample and crystalline Form 5 Compound 1 di(hydrogen sulfate) prepared from exposing crystalline Form 2 Compound 1 di(hydrogen sulfate) trihydrate to 6% RH, as described in Example 9.

FIGS. 48A and 48B show XRPD patterns resulting from the variable temperature study on crystalline Form 2 Compound 1 di(hydrogen sulfate) trihydrate described in Example 9.

FIG. 49 shows XRPD patterns of crystalline Form 2 Compound 1 di(hydrogen sulfate) trihydrate and crystalline Form 5 Compound 1 di(hydrogen sulfate) resulting from heating crystalline Form 2 Compound 1 di(hydrogen sulfate) trihydrate to 80° C., as described in Example 9.

FIG. 50 shows XRPD patterns taken during the experiments relating to crystalline Compound 1 as described in Example 11, compared to exemplary patterns of Compound 1 Form 1 and Compound 1 Form 2.

FIG. 51 shows an XRPD pattern of crystalline Form 1 Compound 1, as prepared in Example 11.

FIG. 52 shows an XRPD pattern of crystalline Form 2 Compound 1, as prepared in Example 11.

FIG. 53 shows XRPD patterns of the results of the DVS experiment described in Example 11 on crystalline Form 1 Compound 1 compared to the input material.

FIG. 54 shows an XRPD pattern of a sample of crystalline Form 10 Compound 1 mono(hydrogen sulfate) as prepared in Example 13.

FIG. 55 shows a peak picked XRPD pattern of a sample of crystalline Form 10 Compound 1 mono(hydrogen sulfate) as prepared in Example 13.

FIG. 56 shows an XRPD pattern of a sample of amorphous Compound 1 mono(hydrogen sulfate) as prepared in Example 13.

FIG. 57 shows TG/DSC thermograms of crystalline Form 10 Compound 1 mono(hydrogen sulfate) obtained from MEK as described in Example 13.

FIG. 58 shows an XRPD pattern of crystalline Form 4 Compound 1 mono(hydrogen sulfate) prepared in Example 14-damp sample.

FIG. 59 shows an XRPD pattern of crystalline Form 8 Compound 1 mono(hydrogen sulfate), prepared by drying crystalline Form 4 Compound 1 mono(hydrogen sulfate) under reduced pressure as described in Example 14.

FIG. 60 shows an XRPD pattern of crystalline Form 4 Compound 1 mono(hydrogen sulfate) prepared in Example 14-air dried.

FIG. 61 shows a TG/DSC of crystalline Form 4 Compound 1 mono(hydrogen sulfate) prepared in Example 14.

FIG. 62 shows a DSC of crystalline Form 4 Compound 1 mono(hydrogen sulfate) prepared in Example 14.

FIG. 63 shows an FT-IR spectrum of crystalline Form 4 Compound 1 mono(hydrogen sulfate) prepared in Example 14.

FIG. 64 shows a ¹H-NMR spectrum of dissolved crystalline Form 4 Compound 1 mono(hydrogen sulfate) prepared in Example 15.

FIG. 65A shows a DVS isotherm of crystalline Form 4 Compound 1 mono(hydrogen sulfate) prepared in Example 14.

FIG. 65B shows a DVS kinetic plot of crystalline Form 4 Compound 1 mono(hydrogen sulfate) prepared in Example 14.

FIG. 66 shows an FT-IR spectrum of crystalline Form 4 Compound 1 mono(hydrogen sulfate) prepared in Example 14, compared to an exemplary FT-IR spectrum of crystalline Form 2 Compound 1 di(hydrogen sulfate) trihydrate.

FIG. 67 shows a ¹H-NMR spectrum of dissolved crystalline Form 4 Compound 1 mono(hydrogen sulfate) prepared in Example 14, compared to exemplary ¹H-NMR spectra from dissolved Compound 1, dissolved crystalline Form 2 Compound 1 di(hydrogen sulfate) trihydrate, and dissolved crystalline Form 3 Compound 1 di(hydrogen sulfate).

FIG. 68 shows an XRPD pattern of crystalline Form 8 Compound 1 mono(hydrogen sulfate) prepared from a DVS experiment on crystalline Form 4 Compound 1 mono(hydrogen sulfate) described in Example 14, compared to the input material and exemplary patterns of crystalline Form 2 Compound 1 di(hydrogen sulfate) trihydrate, crystalline Form 4 Compound 1 mono(hydrogen sulfate), and crystalline Form 8 Compound 1 mono(hydrogen sulfate).

FIG. 69 shows XRPD patterns of crystalline Form 4 Compound 1 mono(hydrogen sulfate) and crystalline Form 8 Compound 1 mono(hydrogen sulfate) after the variable temperature experiment on crystalline Form 4 Compound 1 mono(hydrogen sulfate) described in Example 14.

FIG. 70 shows TG/DSC results of crystalline Form 8 Compound 1 mono(hydrogen sulfate) prepared in Example 14.

FIG. 71 shows an FT-IR spectrum of crystalline Form 8 Compound 1 mono(hydrogen sulfate) prepared in Example 14.

FIG. 72 shows a ¹H-NMR spectrum of dissolved crystalline Form 8 Compound 1 mono(hydrogen sulfate) prepared in Example 14.

FIG. 73 shows an FT-IR spectrum of crystalline Form 8 Compound 1 mono(hydrogen sulfate) prepared in Example 14, compared to an exemplary FT-IR spectrum of crystalline Form 2 Compound 1 di(hydrogen sulfate) trihydrate.

FIG. 74 shows a ¹H-NMR spectrum of dissolved crystalline Form 8 Compound 1 mono(hydrogen sulfate) prepared in Example 14, compared to exemplary ¹H-NMR spectra of dissolved crystalline Form 2 Compound 1 di(hydrogen sulfate) trihydrate and dissolved crystalline Form 4 Compound 1 mono(hydrogen sulfate).

FIG. 75 shows XRPD results from the 7-day stability studies of crystalline Form 3 Compound 1 di(hydrogen sulfate) described in Example 15, compared to exemplary XPRD patterns for crystalline Form 2 Compound 1 di(hydrogen sulfate) trihydrate, crystalline Form 3 Compound 1 di(hydrogen sulfate), crystalline Form 6 Compound 1 di(hydrogen sulfate), and crystalline Form 9 Compound 1 di(hydrogen sulfate).

FIG. 76 shows XRPD results from the 7-day stability studies of crystalline Form 4 Compound 1 mono(hydrogen sulfate) described in Example 15, compared to exemplary XPRD patterns for crystalline Form 4 Compound 1 mono(hydrogen sulfate) and crystalline Form 8 Compound 1 mono(hydrogen sulfate).

FIG. 77 shows XRPD results from pH Solubility Studies of crystalline Form 2 Compound 1 di(hydrogen sulfate) trihydrate described in Example 16, compared to exemplary XRPD patterns for crystalline Form 2 Compound 1 di(hydrogen sulfate) trihydrate and crystalline Form 4 Compound 1 mono(hydrogen sulfate).

FIG. 78 shows XRPD results from pH Solubility Studies of crystalline Form 3 Compound 1 di(hydrogen sulfate) described in Example 16, compared to exemplary XRPD patterns for crystalline Form 2 Compound 1 di(hydrogen sulfate) trihydrate and crystalline Form 4 Compound 1 mono(hydrogen sulfate).

FIG. 79 shows XRPD results from pH Solubility Studies of crystalline Form 4 Compound 1 mono(hydrogen sulfate) described in Example 16, compared to exemplary XRPD patterns for crystalline Form 2 Compound 1 di(hydrogen sulfate) trihydrate and crystalline Form 4 Compound 1 mono(hydrogen sulfate).

FIG. 80 shows XRPD results from hydration mapping studies described in Example 17-Blends, ACN/Water, 50° C., damp—compared to compared to exemplary XRPD patterns for crystalline Form 10 Compound 1 mono(hydrogen sulfate), crystalline Form 1 Compound 1 di(hydrogen sulfate), crystalline Form 2 Compound 1 di(hydrogen sulfate) trihydrate and crystalline Form 3 Compound 1 di(hydrogen sulfate).

FIG. 81 shows XRPD results from hydration mapping studies described in Example 17-Blends, ACN/Water, 20° C., damp—compared to compared to exemplary XRPD patterns for crystalline Form 1 Compound 1 di(hydrogen sulfate), crystalline Form 2 Compound 1 di(hydrogen sulfate) trihydrate and crystalline Form 3 Compound 1 di(hydrogen sulfate).

FIG. 82 shows XRPD results from hydration mapping studies described in Example 17-Blends, IPA/Water, 50° C., damp—compared to compared to exemplary XRPD patterns for crystalline Form 1 Compound 1 di(hydrogen sulfate), crystalline Form 2 Compound 1 di(hydrogen sulfate) trihydrate, crystalline Form 3 Compound 1 di(hydrogen sulfate) and crystalline Form 4 Compound 1 mono(hydrogen sulfate).

FIG. 83 shows XRPD results from hydration mapping studies described in Example 17-Blends, IPA/Water, 20° C., damp—compared to compared to exemplary XRPD patterns for crystalline Form 1 Compound 1 di(hydrogen sulfate), crystalline Form 2 Compound 1 di(hydrogen sulfate) trihydrate, crystalline Form 3 Compound 1 di(hydrogen sulfate) and crystalline Form 4 Compound 1 mono(hydrogen sulfate).

FIG. 84 shows XRPD results from hydration mapping studies described in Example 17-crystalline Form 2 Compound 1 di(hydrogen sulfate) trihydrate, 50° C., damp—compared to compared to exemplary XRPD patterns for crystalline Form 2 Compound 1 di(hydrogen sulfate) trihydrate, crystalline Form 3 Compound 1 di(hydrogen sulfate) and crystalline Form 6 Compound 1 di(hydrogen sulfate).

FIG. 85 shows XRPD results from hydration mapping studies described in Example 17-crystalline Form 3 Compound 1 di(hydrogen sulfate), 50° C., damp—compared to compared to exemplary XRPD patterns for crystalline Form 1 Compound 1 di(hydrogen sulfate), crystalline Form 2 Compound 1 di(hydrogen sulfate) trihydrate, crystalline Form 3 Compound 1 di(hydrogen sulfate) and crystalline Form 6 Compound 1 di(hydrogen sulfate).

FIG. 86 shows XRPD results from hydration mapping studies described in Example 17-crystalline Form 2 Compound 1 di(hydrogen sulfate) trihydrate, 20° C., damp—compared to compared to an exemplary XRPD pattern for crystalline Form 2 Compound 1 di(hydrogen sulfate) trihydrate.

FIG. 87 shows XRPD results from hydration mapping studies described in Example 17-crystalline Form 3 Compound 1 di(hydrogen sulfate), 20° C., damp—compared to compared to exemplary XRPD patterns for crystalline Form 1 Compound 1 di(hydrogen sulfate), crystalline Form 2 Compound 1 di(hydrogen sulfate) trihydrate, crystalline Form 3 Compound 1 di(hydrogen sulfate) and crystalline Form 6 Compound 1 di(hydrogen sulfate).

FIG. 88 shows XRPD results from hydration mapping studies described in Example 17-Blends, ACN/Water, 50° C., dried-compared to compared to exemplary XRPD patterns for crystalline Form 10 Compound 1 mono(hydrogen sulfate), crystalline Form 2 Compound 1 di(hydrogen sulfate) trihydrate, crystalline Form 3 Compound 1 di(hydrogen sulfate) and crystalline Form 6 Compound 1 di(hydrogen sulfate).

FIG. 89 shows XRPD results from hydration mapping studies described in Example 17-Blends, ACN/Water, 20° C., dried-compared to compared to exemplary XRPD patterns for crystalline Form 2 Compound 1 di(hydrogen sulfate) trihydrate, crystalline Form 3 Compound 1 di(hydrogen sulfate) and crystalline Form 6 Compound 1 di(hydrogen sulfate).

FIG. 90 shows XRPD results from hydration mapping studies described in Example 17-Blends, IPA/Water, 50° C., dried-compared to compared to exemplary XRPD patterns for crystalline Form 10 Compound 1 mono(hydrogen sulfate) and crystalline Form 2 Compound 1 di(hydrogen sulfate) trihydrate.

FIG. 91 shows XRPD results from hydration mapping studies described in Example 17-Blends, IPA/Water, 20° C., dried—compared to compared to exemplary XRPD patterns for crystalline Form 10 Compound 1 mono(hydrogen sulfate) and crystalline Form 2 Compound 1 di(hydrogen sulfate) trihydrate.

FIG. 92 shows XRPD results from hydration mapping studies described in Example 17-crystalline Form 2 Compound 1 di(hydrogen sulfate) trihydrate, 50° C., dried—compared to compared to exemplary XRPD patterns for crystalline Form 2 Compound 1 di(hydrogen sulfate) trihydrate and crystalline Form 3 Compound 1 di(hydrogen sulfate).

FIG. 93 shows XRPD results from hydration mapping studies described in Example 17—crystalline Form 3 Compound 1 di(hydrogen sulfate), 50° C., dried-compared to compared to exemplary XRPD patterns for crystalline Form 1 Compound 1 di(hydrogen sulfate), crystalline Form 2 Compound 1 di(hydrogen sulfate) trihydrate and crystalline Form 3 Compound 1 di(hydrogen sulfate).

FIG. 94 shows XRPD results from hydration mapping studies described in Example 17—crystalline Form 2 Compound 1 di(hydrogen sulfate) trihydrate, 20° C., dried—compared to compared to an exemplary XRPD pattern for crystalline Form 2 Compound 1 di(hydrogen sulfate) trihydrate.

FIG. 95 shows XRPD results from hydration mapping studies described in Example 17—crystalline Form 3 Compound 1 di(hydrogen sulfate), 20° C., dried—compared to compared to exemplary XRPD patterns for crystalline Form 2 Compound 1 di(hydrogen sulfate) trihydrate and crystalline Form 3 Compound 1 di(hydrogen sulfate).

FIG. 96 shows an XRPD pattern overlay of various solid forms of Compound 1 hydrogen sulfate.

FIG. 97 shows a Single Crystal Structure Drawing of crystalline Form 2 Compound 1 di(hydrogen sulfate) trihydrate at 100 K—orientation 1.

FIG. 98 shows Single Crystal Structure Drawing of crystalline Form 2 Compound 1 di(hydrogen sulfate) trihydrate at 100 K—orientation 2.

FIG. 99 shows simulated XRPD patterns for crystalline Form 2 Compound 1 di(hydrogen sulfate) trihydrate from crystallographic data obtained at 100 K and 295 K.

FIG. 100 shows stacked plots of simulated XRPD patterns from crystallographic data obtained at 100 K and 295 K (bottom two patterns) along with the experimental XRPD for crystalline Form 2 Compound 1 di(hydrogen sulfate) trihydrate at 298 K (top pattern).

FIG. 101 shows ENT1 inhibition by Compound 1 (free base) and to Form 2 Compound 1 di(hydrogen sulfate) trihydrate.

FIG. 102 shows ENT1 inhibition by Compound 1 (free base) compared to Form 2 Compound 1 di(hydrogen sulfate) trihydrate after storage at −20° C. for 6 months.

FIG. 103 shows an XRPD pattern of crystalline Form 1 Compound 1 hydrogen chloride.

FIG. 104 shows a peak-picked XRPD pattern of crystalline Form 1 Compound 1 hydrogen chloride.

FIG. 105A shows TG/DSC results of crystalline Form 1 Compound 1 hydrogen chloride prepared in Example 3B.

FIG. 105B shows TG/DSC results of crystalline Form 1 Compound 1 hydrogen chloride prepared in Example 3C.

FIG. 106 shows DSC results of crystalline Form 1 Compound 1 hydrogen chloride.

FIG. 107 shows a DVS isotherm plot of crystalline Form 1 Compound 1 hydrogen chloride.

FIG. 108 shows a DVS kinetic plot of crystalline Form 1 Compound 1 hydrogen chloride.

FIG. 109 shows an XRPD pattern of crystalline Form 1 Compound 1 hydrogen phosphate.

FIG. 110 shows a peak-picked XRPD pattern of crystalline Form 1 Compound 1 hydrogen phosphate.

FIG. 111 shows TG/DSC results of crystalline Form 1 Compound 1 hydrogen phosphate (sample dried from ethanol).

FIG. 112 shows TG/DSC results of crystalline Form 1 Compound 1 hydrogen phosphate (sample from ethanol after 48 hours storage at 40° C./75% RH).

FIG. 113 shows an XRPD pattern of crystalline Form 2 Compound 1 hydrogen phosphate.

FIG. 114 shows a peak-picked XRPD pattern of crystalline Form 2 Compound 1 hydrogen phosphate.

FIG. 115 shows TG/DSC results of crystalline Form 2 Compound 1 hydrogen phosphate (sample dried from MEK).

FIG. 116 shows TG/DSC results of crystalline Form 2 Compound 1 hydrogen phosphate (sample from MEK after 48 hours storage at 40° C./75% RH).

FIG. 117 shows an XRPD pattern of crystalline Form 3 Compound 1 hydrogen phosphate.

FIG. 118 shows a peak-picked XRPD pattern of crystalline Form 3 Compound 1 hydrogen phosphate.

FIG. 119 shows an XRPD pattern of crystalline Form 4 Compound 1 hydrogen phosphate.

FIG. 120 shows a peak-picked XRPD pattern of crystalline Form 4 Compound 1 hydrogen phosphate.

FIG. 121 shows an XRPD pattern of crystalline Form 5 Compound 1 hydrogen phosphate.

FIG. 122 shows a peak-picked XRPD pattern of crystalline Form 5 Compound 1 hydrogen phosphate.

FIG. 123 shows an XRPD pattern of crystalline Form 6 Compound 1 hydrogen phosphate from Example 4B.

FIG. 124 shows a peak-picked XRPD pattern of crystalline Form 6 Compound 1 hydrogen phosphate from Example 4B.

FIG. 125 shows an XRPD pattern of crystalline Form 8 Compound 1 hydrogen phosphate.

FIG. 126 shows a peak-picked XRPD pattern of crystalline Form 8 Compound 1 hydrogen phosphate.

FIG. 127 shows TG/DSC results of crystalline Form 5 Compound 1 hydrogen phosphate.

FIG. 128 shows TG/DSC results of crystalline Form 6 Compound 1 hydrogen phosphate (sample dried from ethanol) from Example 4B.

FIG. 129 shows TG/DSC results of crystalline Form 6 Compound 1 hydrogen phosphate (sample from THE after 48 hours storage at 40° C./75% RH) from Example 4B.

FIG. 130 shows TG/DSC results of crystalline Form 8 Compound 1 hydrogen phosphate.

FIG. 131 shows an XRPD pattern of crystalline Form 6 Compound 1 hydrogen phosphate (after 48 hours storage at 40° C./75% RH) from Example 4C.

FIG. 132 shows TG/DSC results of crystalline Form 6 Compound 1 hydrogen phosphate (after 48 hours storage at 40° C./75% RH)) from Example 4C.

FIG. 133 shows an XRPD pattern of crystalline Form 1 Compound 1 fumarate.

FIG. 134 shows a peak-picked XRPD pattern of crystalline Form 1 Compound 1 fumarate.

FIG. 135 shows an XRPD pattern of crystalline Form 2 Compound 1 fumarate.

FIG. 136 shows a peak-picked XRPD pattern of crystalline Form 2 Compound 1 fumarate.

FIG. 137 shows an XRPD pattern of crystalline Form 3 Compound 1 fumarate.

FIG. 138 shows a peak-picked XRPD pattern of crystalline Form 3 Compound 1 fumarate.

FIG. 139 shows TG/DSC results of crystalline Form 1 Compound 1 fumarate.

FIG. 140 shows TG/DSC results of crystalline Form 2 Compound 1 fumarate.

FIG. 141 shows TG/DSC results of crystalline Form 3 Compound 1 fumarate.

FIG. 142 shows TG/DSC results of a mixture of crystalline Form 2 Compound 1 fumarate and crystalline Form 3 Compound 1 fumarate.

FIG. 143A shows DVS isotherm plot of a mixture of crystalline Form 2 Compound 1 fumarate and crystalline Form 3 Compound 1 fumarate.

FIG. 143B shows DVS kinetic plot of a mixture of crystalline Form 2 Compound 1 fumarate and crystalline Form 3 Compound 1 fumarate.

FIG. 144 shows an XRPD pattern of crystalline Form 1 Compound 1 maleate.

FIG. 145 shows a peak-picked XRPD pattern of crystalline Form 1 Compound 1 maleate.

FIG. 146 shows TG/DSC results of crystalline Form 1 Compound 1 maleate.

FIG. 147 shows TG/DSC results of a mixture of crystalline Form 1 Compound 1 maleate and crystalline Form 2 Compound 1 maleate.

FIG. 148 shows an XRPD pattern of crystalline Form 1 Compound 1 benzoate.

FIG. 149 shows a peak-picked XRPD pattern of crystalline Form 1 Compound 1 benzoate.

FIG. 150 shows an XRPD pattern of crystalline Form 2 Compound 1 benzoate.

FIG. 151 shows a peak-picked XRPD pattern of crystalline Form 2 Compound 1 benzoate.

FIG. 152 shows an XRPD pattern of crystalline Form 3 Compound 1 benzoate.

FIG. 153 shows a peak-picked XRPD pattern of crystalline Form 3 Compound 1 benzoate.

FIG. 154 shows TG/DSC results of crystalline Form 1 Compound 1 benzoate.

FIG. 155 shows TG/DSC results of crystalline Form 2 Compound 1 benzoate.

FIG. 156 shows TG/DSC results of crystalline Form 3 Compound 1 benzoate.

FIG. 157 shows TG/DSC results of a mixture of crystalline Form 1 Compound 1 benzoate, crystalline Form 2 Compound 1 benzoate, and crystalline Form 3 Compound 1 benzoate.

FIG. 158 shows an XRPD pattern of crystalline Form 7 Compound 1 hydrogen phosphate.

FIG. 159 shows a peak-picked XRPD pattern of crystalline Form 7 Compound 1 hydrogen phosphate.

### DESCRIPTION OF THE EMBODIMENTS

### I. Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art. Standard techniques may be used for preparation and analysis of solid forms. As appropriate, procedures involving the use of commercially available kits and reagents are generally carried out in accordance with manufacturer-defined protocols and conditions unless otherwise noted.

Unless otherwise indicated, the following terms have the following meanings:

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

As used herein, the term “administration”, or a variant thereof (e.g. “administering”), means providing the active agent or active ingredient, alone or as part of a pharmaceutically acceptable composition, to the patient in whom/which the condition, symptom, or disease is to be treated or prevented.

The term “adenosine receptor antagonist” refers to a compound that, upon administration to a patient, results in inhibition or down-regulation of a biological activity associated with activation of an adenosine receptor in the patient, including any of the downstream biological effects otherwise resulting from the binding to an adenosine receptor of its natural ligand. Such adenosine receptor antagonists include any agent that can block activation of an adenosine receptor or any of the downstream biological effects of an adenosine receptor activation. See, for example, WO 2023/059817 (inupadenant and hydrochloride salt of inupadenant).

The term “patient” refers to a mammal, such as a human, who/which is awaiting the receipt of, or is receiving medical care, or was/is/will be the object of a medical procedure, or is monitored for the development or progression of a disease, such as a cancer.

The expression “pharmaceutically acceptable” refers to compounds, salts, compositions, dosage forms, and other materials which are useful in preparing a pharmaceutical composition that is suitable for veterinary or human pharmaceutical use.

The expression “pharmaceutically acceptable carrier” or “pharmaceutically acceptable excipient” or “excipient” includes any and all solvents, dispersion media, coatings, antibacterial and antifungal agents, isotonic and absorption delaying agents and the like. The use of such media and agents for pharmaceutically active substances is known in the art. Except insofar as any conventional media or agent is incompatible with the active ingredient, its use in the compositions disclosed herein is contemplated. Additional active ingredients can also be incorporated into the compositions.

The terms “therapeutically effective amount” or “effective amount” or “therapeutically effective dose” or dose of a compound or a composition refer to that amount of the compound or the composition that results in reduction or inhibition of symptoms or a prolongation of survival in a subject (such as a human patient). The results may require multiple doses of the compound or the composition. A therapeutically effective amount may be administered prior to the onset of a disease or disorder for a prophylactic or preventive action. Alternatively, or additionally, a therapeutically effective amount may be administered after initiation of a disease or disorder for a therapeutic action. In one embodiment, the disease or disorder is cancer.

At various places in the present disclosure, variables or parameters are disclosed in groups or ranges. It is specifically intended that the description include each and every individual subcombination of the members of such groups and ranges. For example, an integer in the range of 0 to 10 is specifically intended to individually disclose 0, 1, 2, 3, 4, 5, 6, 7, 8, 9, and 10.

The use of any and all examples, or exemplary language herein, for example “such as” or “including” is intended to merely illustrate better the present disclosure and does not pose a limitation on the scope of the disclosure.

As used herein, “Compound 1” and “Compound 1 (free base)” are used interchangeably and refer to the formula shown below.

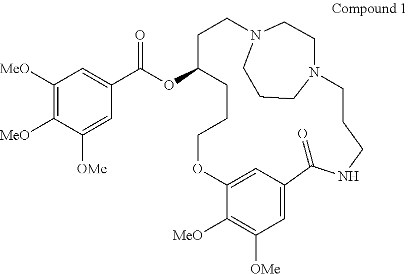 [SMILES: COc1cc(C(=O)O[C@@H]2CCCOc3cc(cc(OC)c3OC)C(=O)NCCCN3CCCN(CC2)CC3)cc(OC)c1OC]

Compound 1 can have the chemical name (16R)-9,10-dimethoxy-6-oxo-12-oxa-1,5,19-triazatricyclo[17.3.2.1^{7,11}]pentacosa-7(25), 8,10-trien-16-yl 3,4,5-trimethoxybenzoate. Compound 1 can also have the chemical name (12R)-7⁴,7⁵-dimethoxy-6-oxo-8-oxa-5-aza-1(1,4)-diazepana-7(1,3)-benzenacyclotetradecaphane-12-yl 3,4,5-trimethoxybenzoate, and this chemical name can be found, for example utilizing ChemDraw® Version 22.0.0.22.

As used herein, “XRPD” refers to X-ray powder diffraction. A typical XRPD pattern is an x-y graph with 20 (diffraction angle) plotted on the x-axis and intensity plotted on the y-axis. These are the diffraction peaks which may be used to characterize a crystalline material. The diffraction peaks are usually represented and referred to by their position on the x-axis rather than the intensity of the diffraction peaks on the y-axis because diffraction peak intensity can be particularly sensitive to sample orientation (see Pharmaceutical Analysis, Lee & Web, pp. 255-257 (2003)). Thus, intensity (sometimes referred to as “Height”) is not typically used by those of skill in the art to characterize a crystalline material. As with any data measurement, there may be variability in XRPD data. In addition to the variability in diffraction peak intensity, there may also be variability in the position of the diffraction peaks on the x-axis. This variability can, however, typically be accounted for when reporting the positions of diffraction peaks for purposes of characterization. Such variability in the position of diffraction peaks along the x-axis may be derived from several sources. One such source can be sample preparation. Samples of the same crystalline material prepared under different conditions may yield slightly different diffractograms. Factors such as particle size, moisture content, solvent content, temperature, and orientation may all affect how a sample diffracts X-rays. Another source of variability comes from instrument parameters. Different X-ray powder diffractometers operate using different parameters and may lead to slightly different diffraction patterns from the same crystalline material. Likewise, different software packages process XRPD data differently and this may also lead to variability. These and other sources of variability are known to those of ordinary skill in the art. Due to such sources of variability, the values of each X-ray diffraction peak are typically preceded with the term “about” or proceeded with an appropriate range defining the experimental variability. For purposes of data reported herein, that value is ±0.2° 2θ unless otherwise stated. This means that on a well-maintained instrument one would expect the variability in peak measurement to be ±0.2° 2θ. X-ray powder diffraction peaks cited herein are generally reported with this variability of ±0.2° 2θ unless stated otherwise and are intended to be reported with such a variability whenever disclosed herein whether the word “about” is present or not, unless context dictates otherwise.

Solid forms, such as those disclosed herein, are readily analyzed by XRPD. The data from X-ray powder diffraction may be used in multiple ways to characterize solid forms. For example, the entire X-ray powder diffraction pattern output from a diffractometer may be used to characterize a solid form. A smaller subset of such data, however, may also be suitable and used for characterizing such solid forms. Indeed, often even a single X-ray powder diffraction peak may be used to characterize a crystalline form.

In some embodiments outside the use of XRPD or as otherwise set forth herein with respect to specific techniques, the term “about” indicates the designated value ±10%, ±5%, or ±1%. In some embodiments, where applicable, the term “about” indicates the designated value(s)±one standard deviation of that value(s).

As used herein, “IR” refers infrared and “FT-IR” refers to Fourier-transform infrared. IR spectroscopies act on the principle that when infrared radiation passes through a sample, some of the radiation is absorbed. The radiation that passes through the sample is recorded. Because different molecules with their different structures produce different spectra, IR (including FT-IR) is a routine technique used to identify and distinguish among molecules. There may be variability in FT-IR data described herein. Due to such sources of variability, the values of each FT-IR peak are typically preceded with the term “about” or proceeded with an appropriate range defining the experimental variability. For purposes of data reported herein, that value is ±4 cm⁻¹ unless otherwise stated. This means that on a well-maintained instrument one would expect the variability in peak measurement to be 4 cm⁻¹. FT-IR peaks cited herein are generally reported with this variability of +4 cm⁻¹ unless stated otherwise and are intended to be reported with such a variability whenever disclosed herein whether the word “about” is present or not, unless context dictates otherwise.

The term “¹H NMR” refers to Proton Nuclear Magnetic Resonance Spectroscopy. Solid-state NMR can be used to study molecules in their solid form. Solution-state NMR can be used to study molecules dissolved in a liquid. Because solution-state NMR is conducted when a molecule is dissolved in a solvent, it provides no data about the solid-state structure (such as a crystal form) but can provide evidence of salt formation due to chemical shift patterns, or hydrate/solvate formation due to the presence of other compounds present in the solution prepared for the NMR analysis.

The term “HPLC-CAD” refers to High Performance Liquid Chromatography-Charged Aerosol Detection. In HPLC-CAD, the CAD (Charged aerosol detector) is used in conjunction with HPLC to measure the amount of chemicals in a sample by creating charged aerosol particles for detection. HPLC-CAD is a well-understood procedure used to obtain a general idea of the quantity of a particular component such as a salt counterion within a compound or material as it is not a precise measure. For example, HPLC-CAD has been used herein to help determine whether, when a salt has formed, whether a hydrogen sulfate salt of Compound 1 is a mono(hydrogen sulfate) salt or di(hydrogen sulfate) salt.

As used herein, the term “one or more peaks” means that any combination of the peaks so given may be present in the corresponding analytical measurement, such as an XRPD pattern. It does not mean (in and of itself) that no other peak may be present in the corresponding analytical measurement, such as an XRPD pattern.

Because hydrates are known to often have greater variability with thermal measurements such as DSC, no explicit definition of “about” is provided herein with respect to thermal measurements.

The term “substantially crystalline” refers to solid forms that may be at least a particular weight percent crystalline. Particular weight percentages may include about 50%, about 75%, about 80%, about 85%, about 87%, about 88%, about 89%, about 90%, about 91%, about 92%, about 93%, about 94%, about 95%, about 96%, about 97%, about 98%, about 99%, about 99.5%, about 99.9%, or any percentage between about 50% and about 100%. In some embodiments, the particular weight is more than about 50%. In some embodiments, the particular weight percent of crystallinity is at least about 90%. In some embodiments, the particular weight percent of crystallinity is at least about 95%.

The term “mono-acid” refers to a molecule that has one transferable hydrogen atom, such as hydrochloric acid (HCl). The term “di-acid” refers to a molecule that has two transferable hydrogen atoms, such as maleic acid.

The term “mono” when referring to a multicomponent complex of Compound 1 (such as Compound 1 mono(hydrogen chloride)), such as a salt, means that the multicomponent complex has a molar ratio of Compound 1 to the coformer (which may be a salt coformer) that is 1:1. Similarly, the term “di” means that the multicomponent complex has a molar ratio of Compound 1 to coformer (which may be the salt coformer) that is 1:2. As used herein, the term coformer means the component of the multicomponent complex which is not Compound 1 (and also not a water or solvent molecule). When the coformer is a salt coformer, then that coformer forms a salt with Compound 1. For example, in Compound 1 di(hydrogen chloride), the coformer is hydrochloric acid and there are two equivalents of hydrochloric acid for each equivalent of Compound 1. In many embodiments, such coformer is a salt coformer and the multicomponent complex is a crystalline complex and, in some embodiments, also a salt.

The multicomponent complexes herein may be identified using their acid coformer name—e.g., Compound 1 fumaric acid, which should be understood to be the same as Compound 1 fumarate.

The term “substantially the same as” refers to results that are, within the experimental variability of the measurements, considered to be equivalent. For example, an XRPD pattern that is substantially the same as another XRPD pattern means the patterns represent the same form of the material as is well understood by one skilled in the art when taking into account, for example, variability associated among samples and instruments, and experimental conditions.

### II. Multicomponent Complexes of Compound 1, Crystalline Forms of Compound 1, and Hydrates and Solvates Thereof

In one aspect, the present disclosure relates to Compound 1 (free base) and hydrates and solvates thereof in crystalline form (also referred to as crystalline Compound 1 and hydrates and solvates thereof).

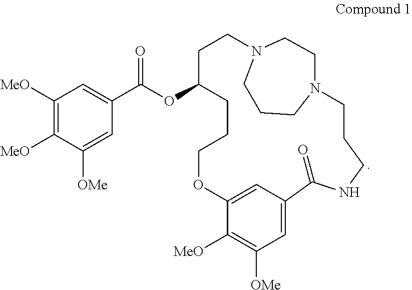 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to multicomponent complexes, such as salts and crystalline complexes, of Compound 1, and hydrates and solvates thereof.

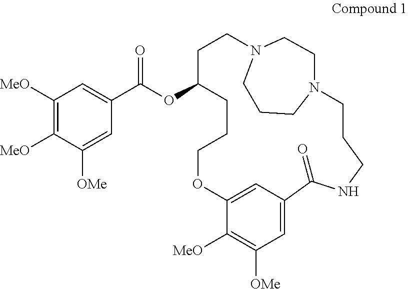 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to a salt of Compound 1 and hydrates and solvates thereof, wherein the salt or hydrate or solvate thereof is not a hexafluorophosphate salt of Compound 1 or a hydrate or solvate thereof.

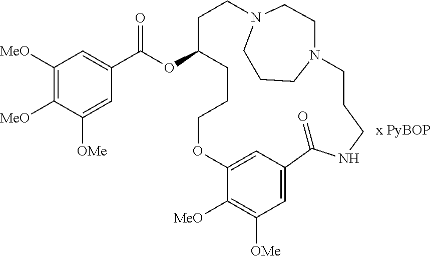 [SMILES: COc1cc(C(=O)O[C@@H]2CCCOc3cc(cc(OC)c3OC)C(=O)NCCCN3CCCN(CC2)CC3)cc(OC)c1OC]

A hexafluorophosphate salt can be formed by, for example, the combination of Compound 1 (free base) and benzotriazol-1-yloxytripyrrolidinophosphonium hexafluorophosphate (PyBOP). Such a salt of Compound 1 can be represented as shown below.

In some embodiments, the present disclosure relates to a salt of Compound 1 and hydrates and solvates of the salt of Compound 1, wherein the salt or hydrate or solvate thereof is not a salt formed from Compound 1 and benzotriazol-1-yloxytripyrrolidinophosphonium hexafluorophosphate (PyBOP).

In one aspect, the present disclosure relates to a pharmaceutically acceptable salt of Compound 1, and hydrates and solvates thereof.

What is meant by “a salt of Compound 1” is a multicomponent complex comprising an acid (the salt coformer) with Compound 1 (which is a free base). For example, in many embodiments, a hydrogen chloride salt of Compound 1 would be a multicomponent complex containing Compound 1 interacting with a hydrogen chloride such that the result would comprise a Compound 1 cation interacting with a chloride anion. The salt may further comprise water or a solvent in which case the salt is also a hydrate or solvate respectively. The salt could be both if both water and a solvent are present. The salt could also be a cocrystal in the sense that one coformer molecule is a salt moiety such that there is proton transfer, and another does not donate a proton such that it is neutral. In such circumstances, the coformers could have the same or different molecular structure. In some embodiments, there is no evidence of proton transfer for any coformer.

As an example, the term “Compound 1 mono(hydrogen chloride)” means, in many embodiments for example, a salt which has been formed by the combination of Compound 1 and hydrochloric acid such that the molar ratio of Compound 1 to hydrogen chloride is 1:1. Likewise, the term “Compound 1 di(hydrogen chloride)” means, in many embodiments for example, a salt that has been formed by the combination of Compound 1 and hydrochloric acid such that the molar ratio of Compound 1 to hydrogen chloride is 1:2.

The ratio of Compound 1 to coformer is not meant to be limited to exactly precisely 1:1 or 1:2. Persons of skill in the art would readily understand the variability associated with different samples of these multicomponent complexes, along with the variability associated with the experimental measurement of the amount of coformer in the multicomponent complex.

When illustrating the multicomponent complex one may, for example, refer to Compound 1 hydrogen chloride as “Compound 1× nHCl” where n is a number greater than 0 with “x” representing an interaction such that the corresponding material is multicomponent.

The term “salt” encompasses salts that are solvates, hydrates, and cocrystals. In many embodiments, the disclosure is directed to solvates and hydrates of Compound 1 salts such as hydrates of Compound 1 hydrogen chloride, including various crystalline forms of Compound 1 hydrogen chloride hydrates. Hydrates may be stoichiometric or non-stoichiometric. For example, when written in the following way: Compound 1 di(hydrogen chloride) trihydrate or Compound 1×2HCl×3H₂O, what is meant is a trihydrate of the di(hydrogen chloride) salt of Compound 1.

In one aspect, the present disclosure relates to a multicomponent complex of Compound 1 and hydrogen sulfate. By “Compound 1 hydrogen sulfate” what is meant is a multicomponent complex comprising hydrogen sulfate anion and/or sulfuric acid (neutral) with Compound 1 which, in many embodiments, is a salt. Such a multicomponent complex could be a hydrate or solvate. In such complexes, the ratio of hydrogen sulfate anion and/or sulfuric acid may be an integer (e.g., mono-, di-, etc.) or a non-integer (e.g., hemi-, sesqui-, or other fractions). Indeed, the ratio of Compound 1 to hydrogen sulfate anion and/or sulfuric acid is not meant to be limited to exactly precisely 1:1 or 1:2. Persons of skill in the art would readily understand the variability associated with different samples of these multicomponent complexes, along with the variability associated with the experimental measurement of the amount of coformer in the multicomponent complex.

The term “Compound 1 mono(hydrogen sulfate)” means, in many embodiments for example, a salt which has been formed by the combination of Compound 1 (free base) and one molecular equivalent of sulfuric acid. Likewise, the term “Compound 1 di(hydrogen sulfate)” means, in many embodiments for example, a salt that has been formed by the combination of Compound 1 and two molecular equivalents of sulfuric acid. When illustrating the multicomponent complex, such as a salt, one may, for example, refer to Compound 1 hydrogen sulfate as “Compound 1×nH₂SO₄” where n is a number greater than 0 with “x” representing an interaction such that the corresponding material is multicomponent.

When n is 2, one of ordinary skill would recognize that such a combination could also be referred to as a “bis(hydrogen sulfate)” complex with Compound 1, and that “bis(hydrogen sulfate)” and di(hydrogen sulfate) are understood to mean the same thing.

In many embodiments, the disclosure is directed to hydrates of Compound 1 such as hydrates of Compound 1 hydrogen sulfate, including various crystalline forms of Compound 1 hydrogen sulfate hydrates. Hydrates may be stoichiometric or non-stoichiometric. For example, when written in the following way: Compound 1 di(hydrogen sulfate) trihydrate or Compound 1×2H₂SO₄×3H₂O, what is meant is a trihydrate of the di(hydrogen sulfate) salt of Compound 1.

In many embodiments, the disclosure is directed to solvates of Compound 1 such as solvates of Compound 1 hydrogen sulfate, including various crystalline forms of Compound 1 hydrogen sulfate solvates. Solvates may be stoichiometric or non-stoichiometric.

In one aspect, the present disclosure relates to a crystalline complex comprising Compound 1 and a coformer, and hydrates and solvates of the crystalline complex.

A “crystalline complex” as used herein refers to a crystalline multicomponent complex comprising two or more molecules, such as, for example, Compound 1 and a cofomer or a water molecule or both. A crystalline complex can be, but is not limited to, crystalline salts.

In one aspect, the present disclosure relates to a crystalline salt of Compound 1 comprising Compound 1 and a salt coformer, and hydrates and solvates of the crystalline salt.

Compound 1 (free base) can be prepared as described in, for example, WO 2021/204896. Compound 1 can also be prepared as shown in Scheme 1, below:

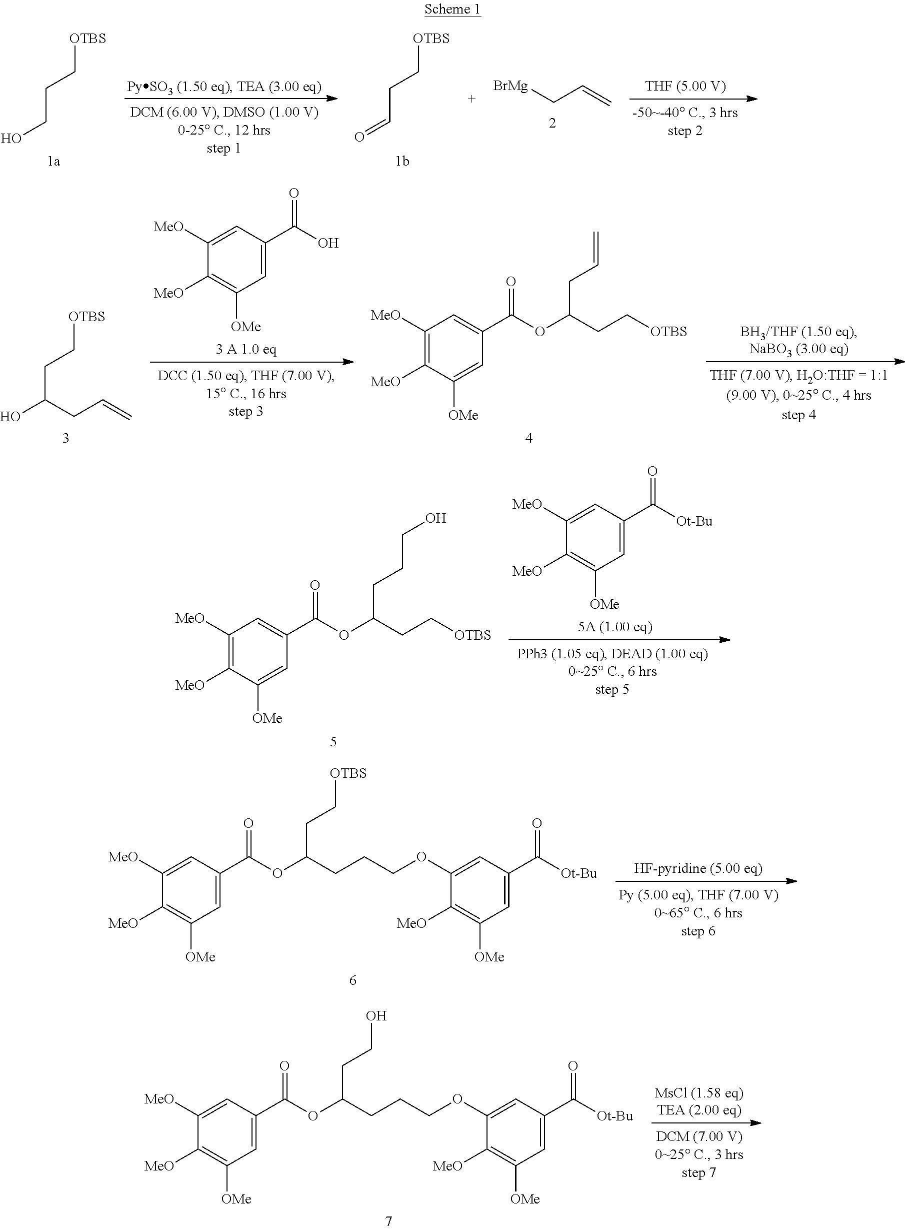

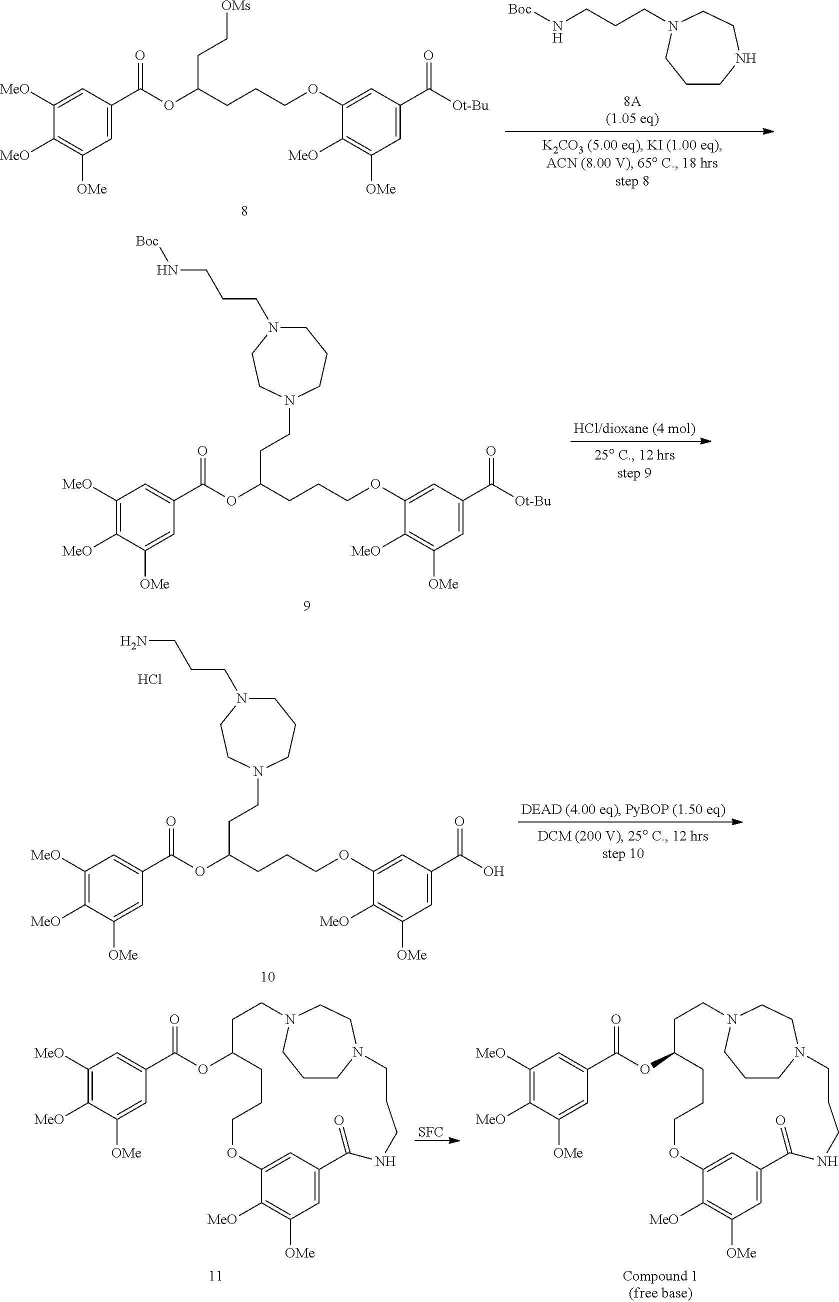

Compound 1 can also be prepared as shown in Scheme 2, below:

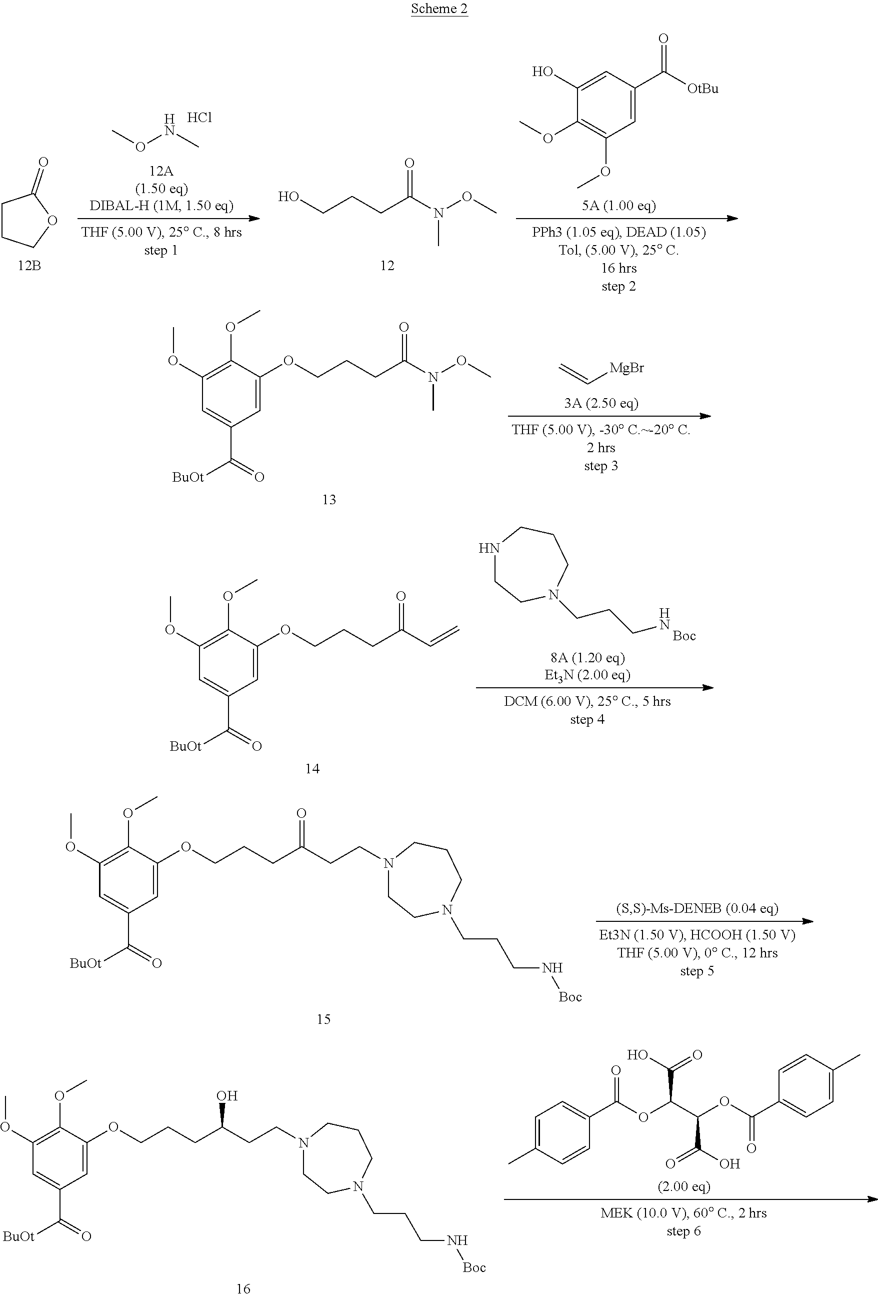

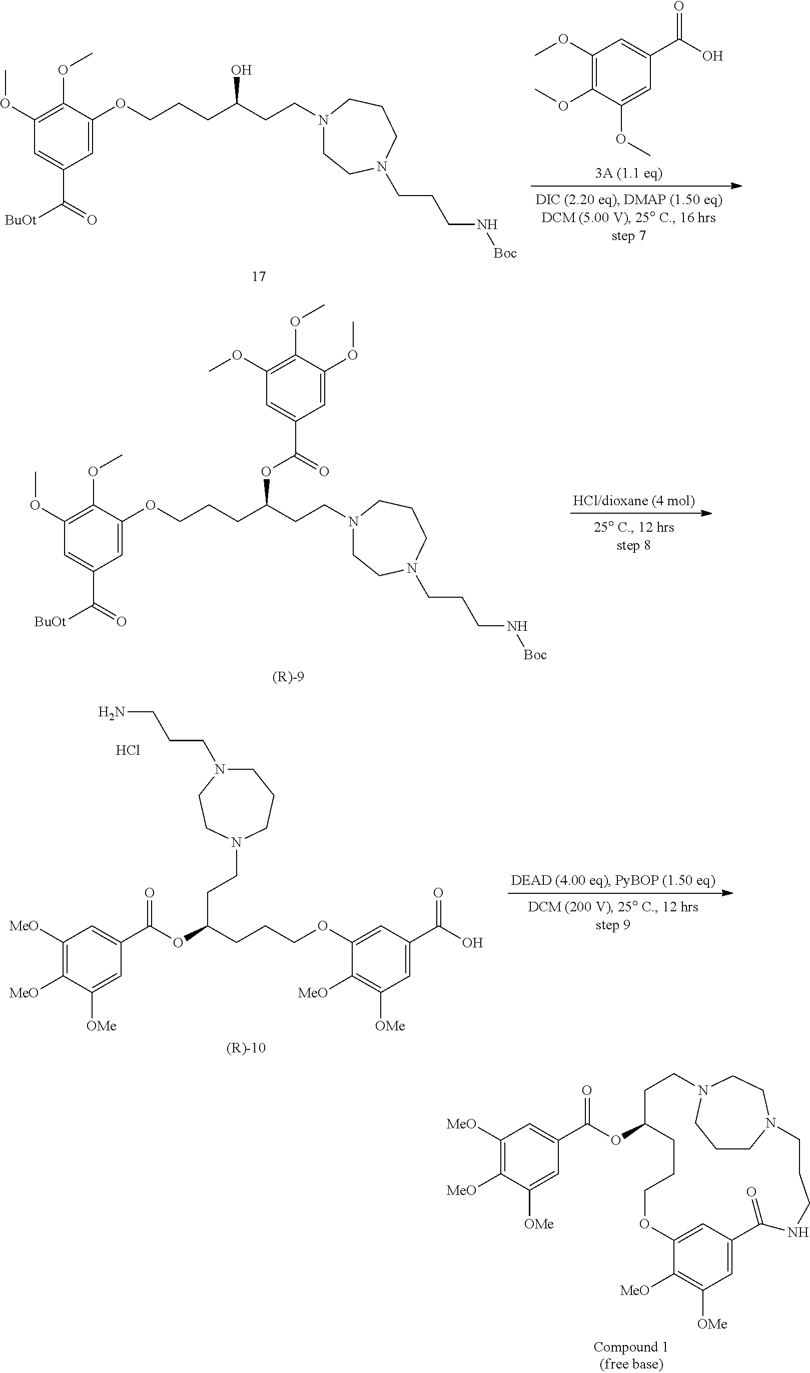

Compound 1 can also be prepared as shown in Scheme 3, below:

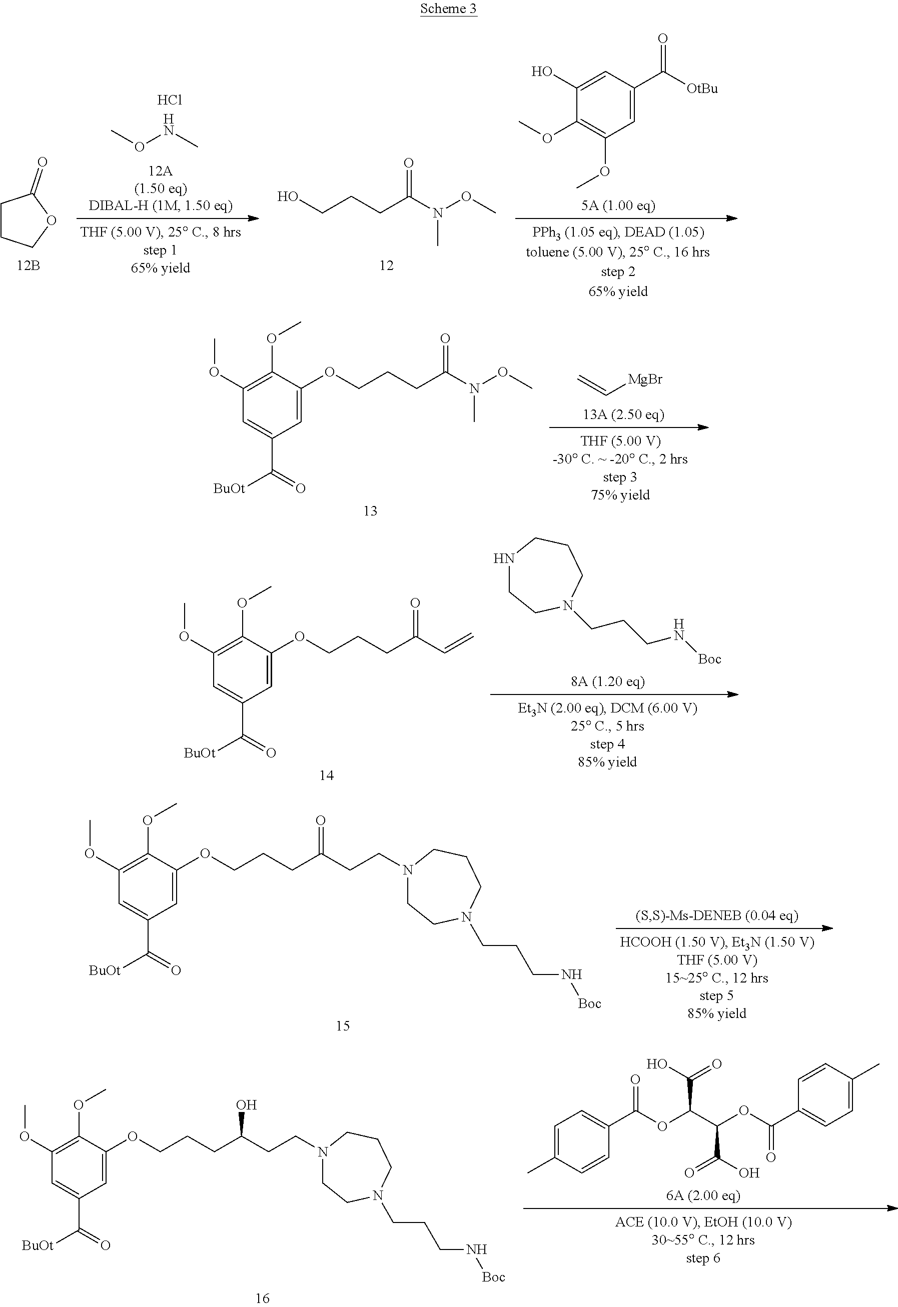

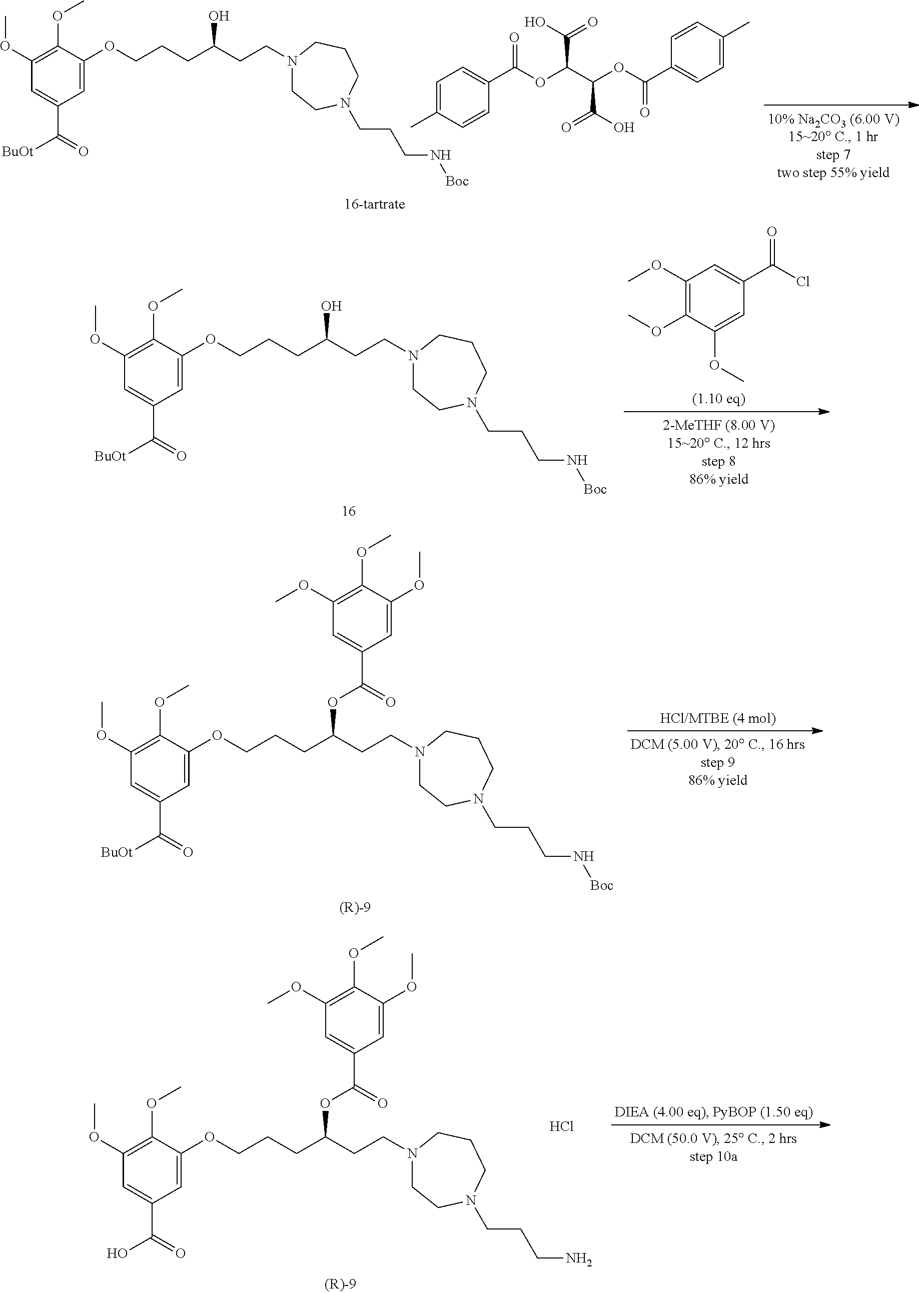

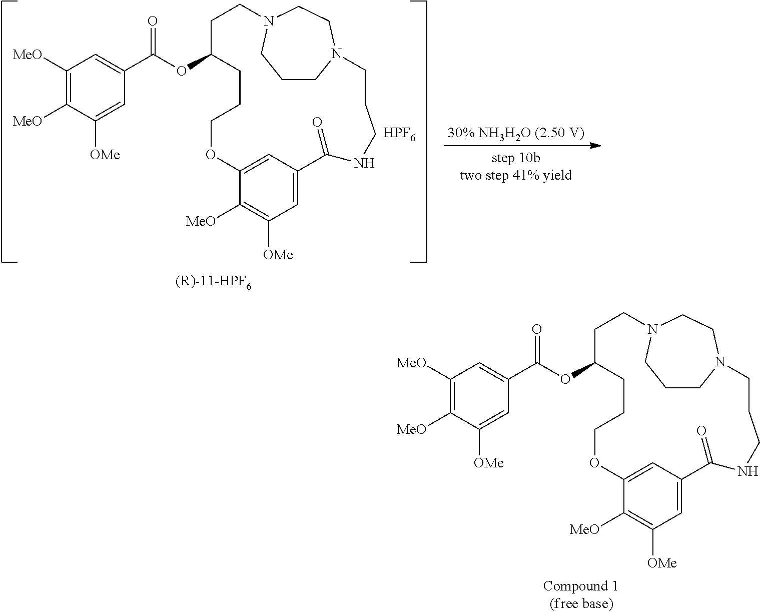

Compound 1 can also be prepared as shown in Scheme 4, below:

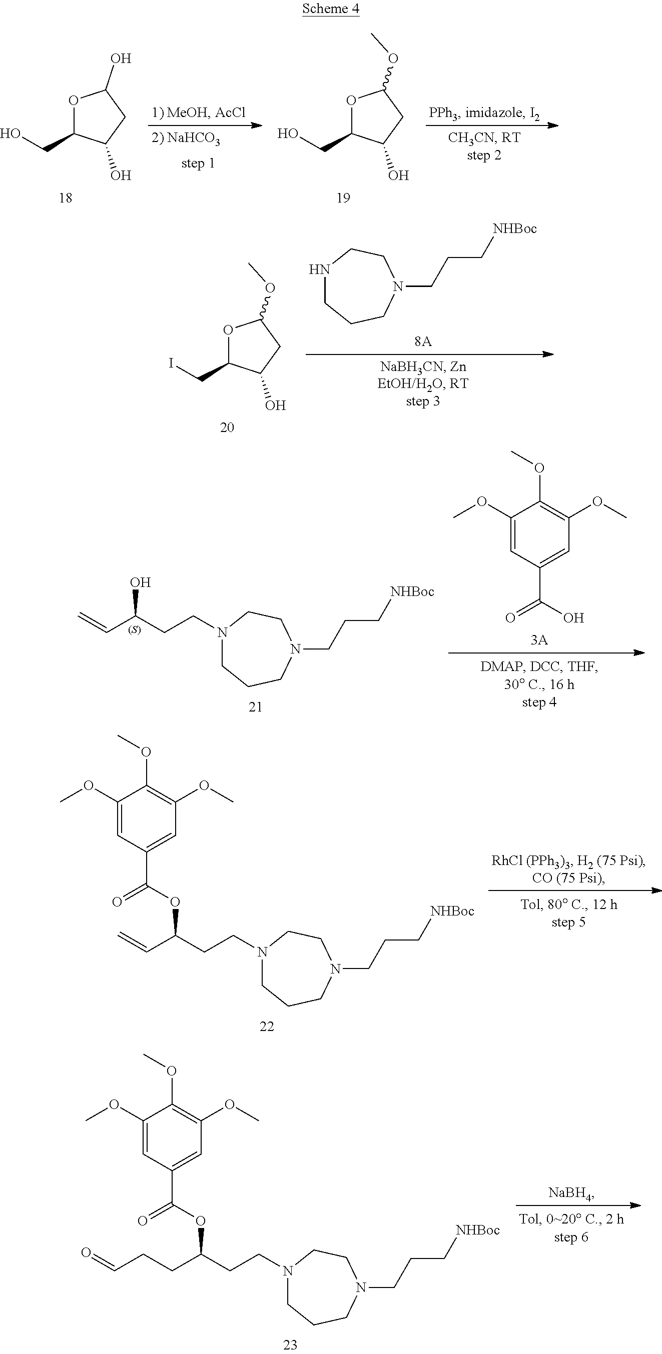

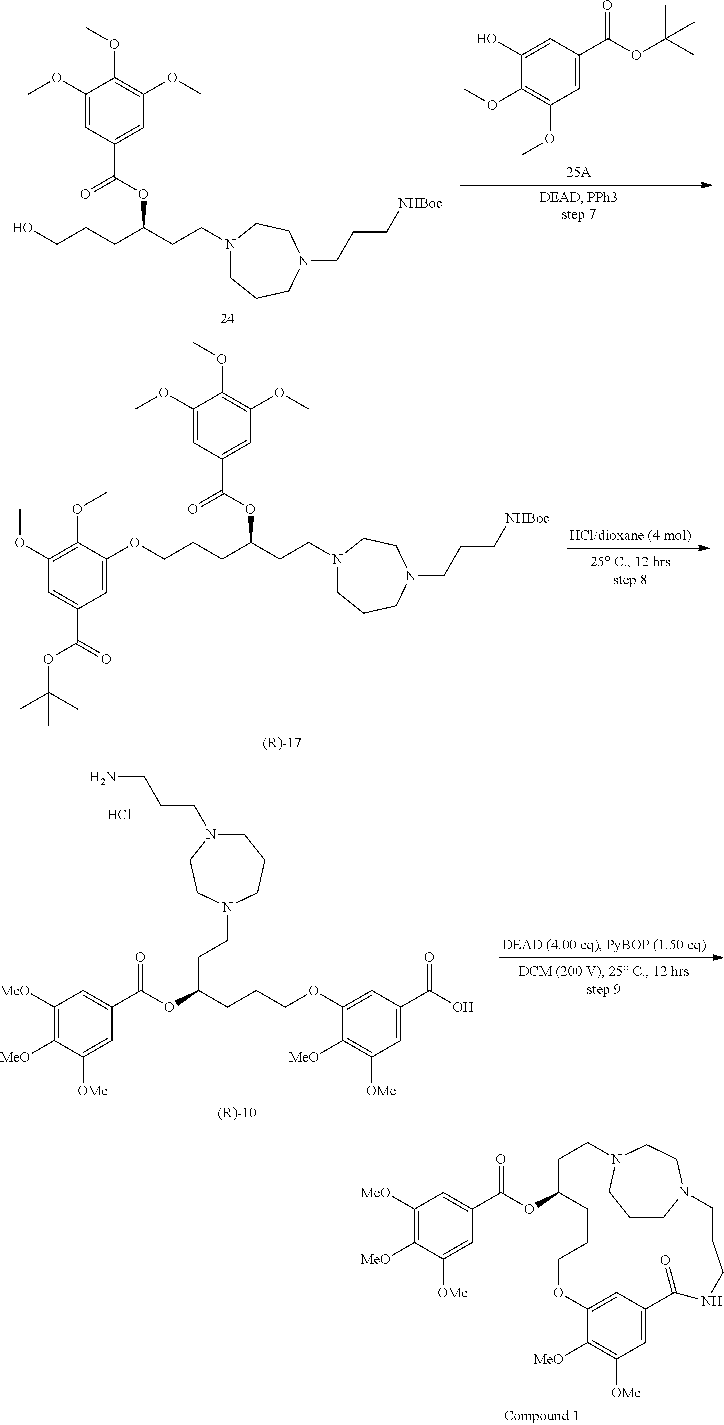 [SMILES: COc1cc(C(=O)O[C@H](CCCO)CCN2CCCN(CCCNC(=O)OC(C)(C)C)CC2)cc(OC)c1OC ; COc1cc(C(=O)OC(C)(C)C)cc(O)c1OC ; COc1cc(C(=O)O[C@H](CCCOc2cc(C(=O)OC(C)(C)C)cc(OC)c2OC)CCN2CCCN(CCCNC(=O)OC(C)(C)C)CC2)cc(OC)c1OC ; COc1cc(C(=O)O[C@H](CCCOc2cc(C(=O)O)cc(OC)c2OC)CCN2CCCN(CCCN)CC2)cc(OC)c1OC ; Cl ; COc1cc(C(=O)O[C@@H]2CCCOc3cc(cc(OC)c3OC)C(=O)NCCCN3CCCN(CC2)CC3)cc(OC)c1OC]

### (1) Multicomponent Complexes of Compound 1 and Hydrates and Solvates Thereof

In one aspect, the present disclosure relates to a salt of Compound 1 and hydrates and solvates thereof, wherein the salt or hydrate or solvate thereof is not a hexafluorophosphate salt of Compound 1 or a hydrate or solvate thereof. In some embodiments, the present disclosure relates to a salt of Compound 1 and hydrates and solvates of the salt of Compound 1, wherein the salt or a hydrate or solvate thereof is not a salt formed from Compound 1 and benzotriazol-1-yloxytripyrrolidinophosphonium hexafluorophosphate (PyBOP).

In one aspect, the present disclosure relates to organic salts and crystalline complexes of Compound 1. In one aspect, the present disclosure relates to inorganic salts and crystalline complexes of Compound 1, wherein the salt or a hydrate or solvate thereof is not a hexafluorophosphate salt of Compound 1 or a hydrate or solvate thereof.

In some embodiments, the salt of Compound 1 or hydrate or solvate thereof is a mono(salt). In some embodiments, the salt of Compound 1 or hydrate or solvate thereof is a di(salt).

In some embodiments, the salt of Compound 1 or hydrate or solvate thereof is selected from Compound 1 hydrogen chloride, Compound 1 hydrogen sulfate, Compound 1 hydrogen phosphate, Compound 1 tosylate, Compound 1 mesylate, Compound 1 benzenesulfonate, Compound 1 L-tartrate, Compound 1 fumarate, Compound 1 citrate, Compound 1 maleate, Compound 1 L-malate, Compound 1 benzoate, and hydrates and solvates thereof.

In one aspect, the present disclosure relates to a pharmaceutically acceptable salt of Compound 1 and hydrates and solvates thereof. In some embodiments, the pharmaceutically acceptable salt or hydrate or solvate thereof is crystalline.

In one aspect, the present disclosure relates to a crystalline complex comprising Compound 1 and a coformer, and hydrates and solvates of the crystalline complex. In some embodiments, the coformer is a neutral coformer. In some embodiments, the coformer is a salt coformer. In some embodiments, the salt coformer is an inorganic acid. In some embodiments, the salt coformer is sulfuric acid. In some embodiments, the salt coformer is hydrochloric acid or phosphoric acid. In some embodiments, the salt coformer is hydrochloric acid. In some embodiments, the salt coformer is phosphoric acid. In some embodiments, the salt coformer is an organic acid. In some embodiments, the salt coformer is an aromatic acid. In some embodiments, the salt coformer is benzoic acid. In some embodiments, the salt coformer is a di-acid. In some embodiments, the di-acid contains at least one hydroxyl group. In some embodiments, the salt coformer is L-malic acid. In some embodiments, the salt coformer is fumaric acid. In some embodiments, the salt coformer is not hexafluorophosphoric acid. In some embodiments, the salt coformer is not benzotriazole-1-yloxytripyrrolidinophosphonium hexafluorophosphate (PyBOP).

In one aspect, the present disclosure relates to a crystalline salt of Compound 1 comprising Compound 1 and a salt coformer, and hydrates and solvates of the crystalline salt. In some embodiments, the salt coformer is an inorganic acid. In some embodiments, the salt coformer is sulfuric acid. In some embodiments, the salt coformer is hydrochloric acid or phosphoric acid. In some embodiments, the salt coformer is hydrochloric acid. In some embodiments, the salt coformer is phosphoric acid. In some embodiments, the salt coformer is an organic acid. In some embodiments, the salt coformer is an aromatic acid. In some embodiments, the salt coformer is benzoic acid. In some embodiments, the salt coformer is a di-acid. In some embodiments, the di-acid contains at least one hydroxyl group. In some embodiments, the salt coformer is L-malic acid. In some embodiments, the salt coformer is fumaric acid. In some embodiments, the salt coformer is not hexafluorophosphoric acid. In some embodiments, the salt coformer is not benzotriazole-1-yloxytripyrrolidinophosphonium hexafluorophosphate (PyBOP).

In some embodiments, the salt coformer of the crystalline salt of Compound 1 or hydrate or solvate thereof is a mono(salt coformer) salt or hydrate or solvate thereof. In some embodiments, the salt coformer of the crystalline salt of Compound 1 or hydrate or solvate thereof is a di(salt coformer) salt or hydrate or solvate thereof.

In one aspect, the present disclosure relates to an amorphous salt of Compound 1 comprising Compound 1 and a salt coformer, and hydrates and solvates of said amorphous salt. In some embodiments, the salt coformer is sulfuric acid. In some embodiments, the salt coformer is selected from hydrochloric acid, p-toluenesulfonic acid, phosphoric acid, methanesulfonic acid, benzenesulfonic acid, L-tartaric acid, fumaric acid, citric acid, maleic acid, L-malic acid, and benzoic acid. In some embodiments, the salt coformer is selected from p-toluenesulfonic acid, methanesulfonic acid, and benzenesulfonic acid. In some embodiments, the salt coformer is not hexafluorophosphoric acid. In some embodiments, the salt coformer is not benzotriazole-1-yloxytripyrrolidinophosphonium hexafluorophosphate (PyBOP).

In some embodiments, the amorphous salt or hydrate or solvate thereof is a mono(salt coformer) salt or hydrate or solvate thereof. In some embodiments, the amorphous salt or hydrate or solvate thereof is a di(salt coformer) salt or hydrate or solvate thereof.

In one aspect, the present disclosure relates to Compound 1 hydrogen sulfate and hydrates and solvates thereof. Compound 1 hydrogen sulfate is formed by, for example, the combination of Compound 1 (free base) and sulfuric acid and can be represented as shown below where n is a number greater than zero.

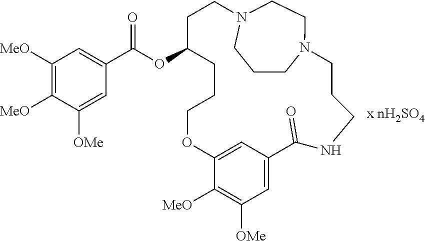 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 mono(hydrogen sulfate) and hydrates and solvates thereof. Compound 1 mono(hydrogen sulfate) is formed by, for example, the combination of Compound 1 (free base) and one molecular equivalent of sulfuric acid and can be represented as shown below.

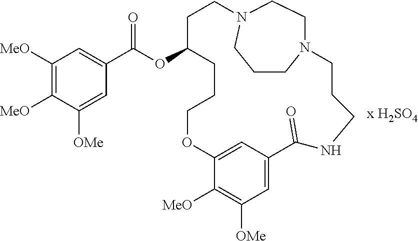 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 di(hydrogen sulfate) and hydrates and solvates thereof. Compound 1 di(hydrogen sulfate) is formed by, for example, the combination of Compound 1 (free base) and two molecular equivalents of sulfuric acid and can be represented as shown below.

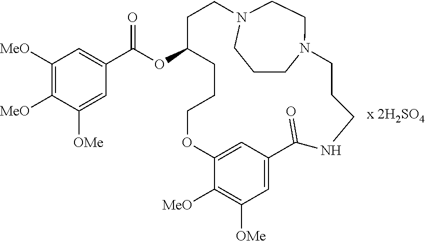 [SMILES: COc1cc(C(=O)O[C@@H]2CCCOc3cc(cc(OC)c3OC)C(=O)NCCCN3CCCN(CC2)CC3)cc(OC)c1OC]

In some embodiments, the Compound 1 hydrogen sulfate or hydrate or solvate thereof is a Compound 1 hydrogen sulfate. In some embodiments, the Compound 1 hydrogen sulfate or hydrate or solvate thereof is a Compound 1 hydrogen sulfate hydrate or solvate.

In some embodiments, the Compound 1 hydrogen sulfate is crystalline. In some embodiments, the Compound 1 hydrogen sulfate hydrate or solvate is crystalline.

In some embodiments, the Compound 1 hydrogen sulfate or hydrate or solvate thereof is a mono(hydrogen sulfate) or a hydrate or solvate thereof. In some embodiments, the Compound 1 mono(hydrogen sulfate) or hydrate or solvate thereof is a Compound 1 mono(hydrogen sulfate). In some embodiments, the Compound 1 mono(hydrogen sulfate) or hydrate or solvate thereof is a Compound 1 mono(hydrogen sulfate) hydrate or solvate.

In some embodiments, the Compound 1 mono(hydrogen sulfate) is crystalline. In some embodiments, the Compound 1 mono(hydrogen sulfate) hydrate or solvate is crystalline.

In some embodiments, the Compound 1 hydrogen sulfate or hydrate or solvate thereof is a di(hydrogen sulfate) or a hydrate or solvate thereof. In some embodiments, the Compound 1 di(hydrogen sulfate) or hydrate or solvate thereof is a Compound 1 di(hydrogen sulfate). In some embodiments, the Compound 1 di(hydrogen sulfate) or hydrate or solvate thereof is a Compound 1 di(hydrogen sulfate) hydrate or solvate. In some embodiments, the Compound 1 di(hydrogen sulfate) hydrate is a trihydrate.

In some embodiments, the Compound 1 di(hydrogen sulfate) is crystalline. In some embodiments, the Compound 1 di(hydrogen sulfate) hydrate or solvate is crystalline. In some embodiments, the Compound 1 di(hydrogen sulfate) trihydrate is crystalline.

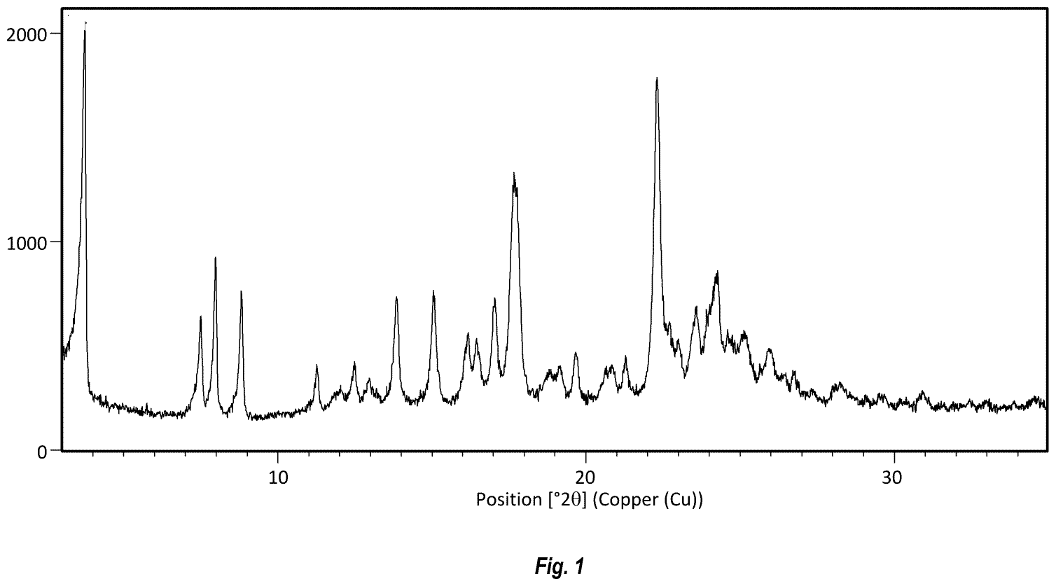

In one aspect, the present disclosure relates to Compound 1 hydrogen sulfate or a hydrate or solvate thereof, including crystalline Form 1 Compound 1 di(hydrogen sulfate) or a hydrate or solvate thereof, having an X-ray powder diffraction pattern comprising a peak at about 3.7° 2θ. In some embodiments, the Compound 1 hydrogen sulfate or hydrate or solvate thereof, including crystalline Form 1 Compound 1 di(hydrogen sulfate) or a hydrate or solvate thereof, has an X-ray powder diffraction pattern comprising one or more peaks chosen from peaks at about 3.7° 2θ, about 7.5° 2θ, about 8.0° 2θ, about 8.8° 2θ, about 11.3° 2θ, about 15.1° 2θ, and about 16.2° 2θ. In some embodiments, the Compound 1 hydrogen sulfate or hydrate or solvate thereof, including crystalline Form 1 Compound 1 di(hydrogen sulfate) or a hydrate or solvate thereof, has an X-ray powder diffraction pattern comprising peaks at about 3.7° 2θ, and about 15.1° 2θ. In some embodiments, the Compound 1 hydrogen sulfate or hydrate or solvate thereof, including crystalline Form 1 Compound 1 di(hydrogen sulfate) or a hydrate or solvate thereof, has an X-ray powder diffraction pattern substantially the same as that of FIG. 1.

In one aspect, the present disclosure relates to crystalline Form 1 Compound 1 di(hydrogen sulfate) and hydrates and solvates thereof. In some embodiments, the crystalline Form 1 Compound 1 di(hydrogen sulfate) or hydrate or solvate thereof has an X-ray powder diffraction pattern comprising one or more peaks chosen from the peaks listed in Table 1 below (all peaks that can be or have been chosen from Table 1 are rounded to the nearest 0.1° 2θ). In some embodiments, the crystalline Form 1 Compound 1 di(hydrogen sulfate) or hydrate or solvate thereof has an X-ray powder diffraction pattern comprising two or more peaks chosen from the peaks listed in Table 1 below (all peaks that can be or have been chosen from Table 1 are rounded to the nearest 0.1° 2θ). In some embodiments, the crystalline Form 1 Compound 1 di(hydrogen sulfate) or hydrate or solvate thereof has an X-ray powder diffraction pattern comprising three or more peaks chosen from the peaks listed in Table 1 below (all peaks that can be or have been chosen from Table 1 are rounded to the nearest 0.1° 2θ).

TABLE 1
Peak list for crystalline Form 1 Compound 1 di(hydrogen sulfate)

| No. | Pos. [°2θ] | d-spacing [Å] | Height [cts] | Rel. Int. [%] |
|---|---|---|---|---|
| 1 | 3.7464 | 23.58479 | 1860.17 | 100 |
| 2 | 7.5083 | 11.77445 | 482.43 | 25.93 |
| 3 | 7.9885 | 11.06769 | 767.94 | 41.28 |
| 4 | 8.8288 | 10.01609 | 600.06 | 32.26 |
| 5 | 11.2762 | 7.84713 | 235.47 | 12.66 |
| 6 | 12.0141 | 7.36674 | 119.08 | 6.4 |
| 7 | 12.4955 | 7.08398 | 247.85 | 13.32 |
| 8 | 12.9476 | 6.83763 | 171.08 | 9.2 |
| 9 | 13.8564 | 6.39118 | 572.78 | 30.79 |
| 10 | 15.0591 | 5.88332 | 551.54 | 29.65 |
| 11 | 16.1731 | 5.4805 | 398.87 | 21.44 |
| 12 | 16.4445 | 5.39066 | 357.09 | 19.2 |
| 13 | 17.0583 | 5.19807 | 514.36 | 27.65 |
| 14 | 17.6424 | 5.02726 | 1112.31 | 59.8 |
| 15 | 19.1684 | 4.63033 | 218.06 | 11.72 |
| 16 | 19.671 | 4.51316 | 291.1 | 15.65 |
| 17 | 20.8581 | 4.25889 | 217.49 | 11.69 |
| 18 | 21.3039 | 4.17079 | 250.83 | 13.48 |
| 19 | 22.2867 | 3.98903 | 1620.88 | 87.14 |
| 20 | 23.581 | 3.77293 | 492.38 | 26.47 |
| 21 | 24.2893 | 3.66448 | 644.33 | 34.64 |
| 22 | 25.1545 | 3.54038 | 369.26 | 19.85 |
| 23 | 25.9599 | 3.43234 | 294.13 | 15.81 |
| 24 | 26.7873 | 3.32817 | 155.01 | 8.33 |
| 25 | 28.1741 | 3.16742 | 110.28 | 5.93 |
| 26 | 30.8859 | 2.89522 | 61.93 | 3.33 |

In one aspect, the present disclosure relates to Compound 1 hydrogen sulfate or a hydrate or solvate thereof, including crystalline Form 2 Compound 1 di(hydrogen sulfate) trihydrate, having an X-ray powder diffraction pattern comprising a peak at about 4.9° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ and about 9.9° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ and about 11.7° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ and about 15.0° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ, about 11.7° 2θ, and about 15.0° 2θ.

In some embodiments, the Compound 1 hydrogen sulfate or hydrate or solvate thereof, including crystalline Form 2 Compound 1 di(hydrogen sulfate) trihydrate, has an X-ray powder diffraction pattern comprising one or more peaks chosen from about 4.9° 2θ, about 9.9° 2θ about 10.2° 2θ, about 11.7° 2θ, about 12.7° 2θ, about 14.4° 2θ, about 15.0° 2θ, about 15.7° 2θ, about 19.0° 2θ, and about 19.6° 2θ.

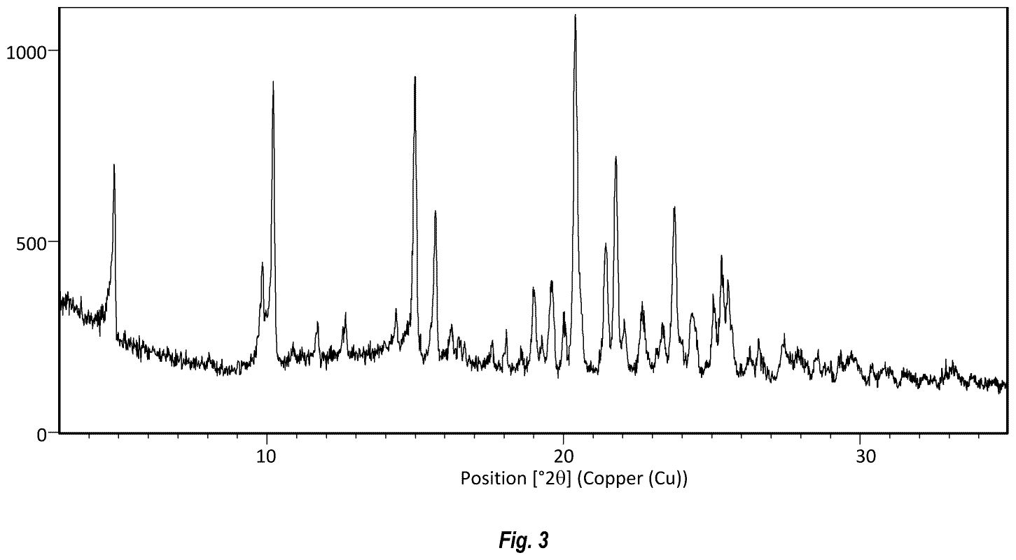

In some embodiments, the Compound 1 hydrogen sulfate or hydrate or solvate thereof, including crystalline Form 2 Compound 1 di(hydrogen sulfate) trihydrate, has an X-ray powder diffraction pattern comprising a peak at about 4.9° 2θ and one or more peaks chosen from peaks at about 10.2° 2θ and about 15.0° 2θ. In some embodiments, the Compound 1 hydrogen sulfate or hydrate or solvate, including crystalline Form 2 Compound 1 di(hydrogen sulfate) trihydrate, has an X-ray powder diffraction pattern substantially the same as that of FIG. 3.

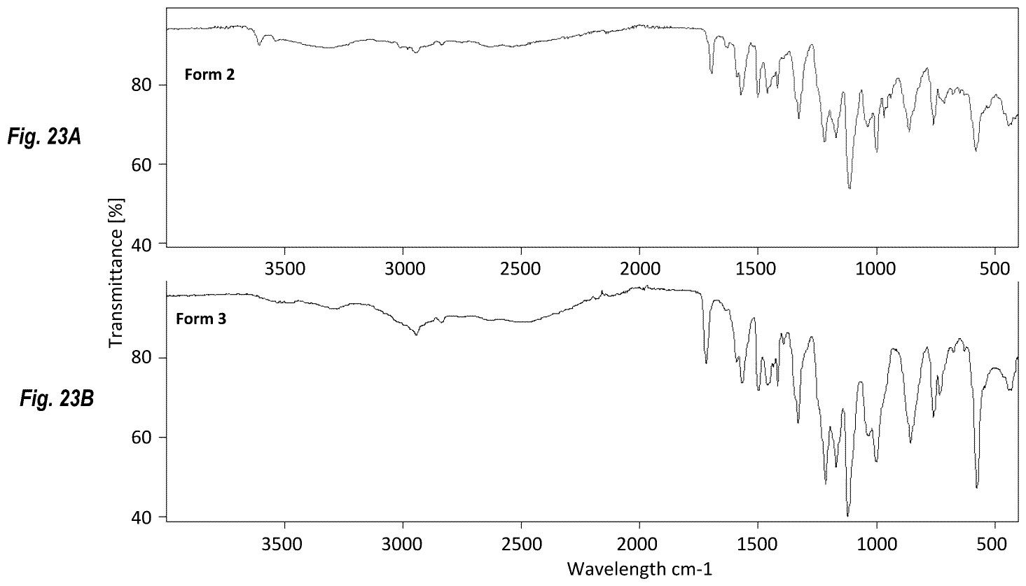

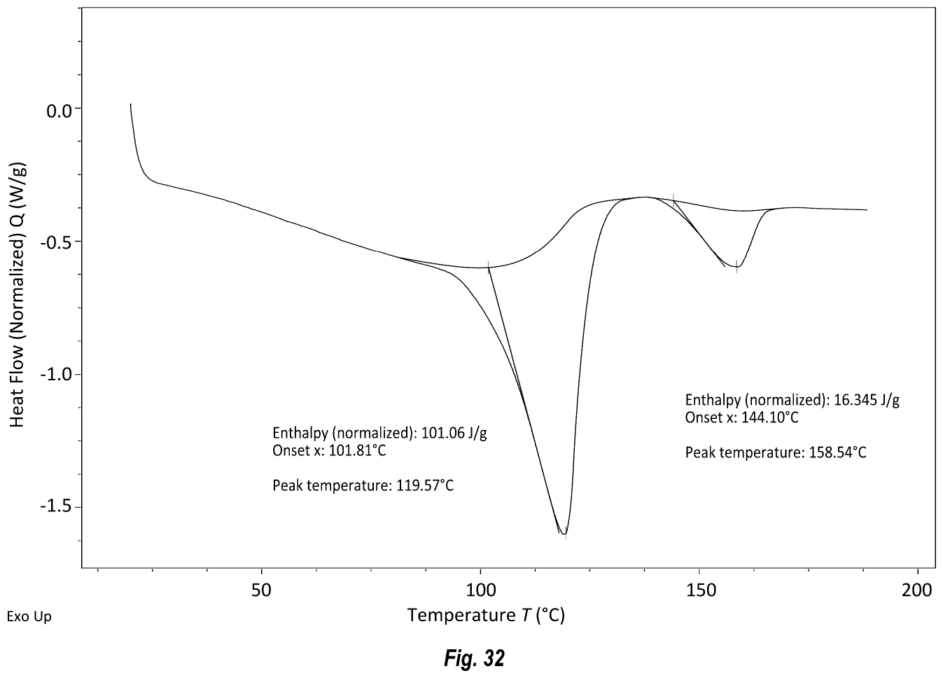

In some embodiments, the Compound 1 hydrogen sulfate or hydrate or solvate thereof, including crystalline Form 2 Compound 1 di(hydrogen sulfate) trihydrate, has an FT-IR spectrum substantially the same as that of FIG. 23A. In some embodiments, the Compound 1 hydrogen sulfate or hydrate or solvate thereof, including crystalline Form 2 Compound 1 di(hydrogen sulfate) trihydrate, has a melting onset as measured by DSC in a sealed aluminum pan with a pierced lid of about 144° C. In some embodiments, the Compound 1 hydrogen sulfate or hydrate or solvate thereof, including crystalline Form 2 Compound 1 di(hydrogen sulfate) trihydrate, has a DSC thermogram substantially the same as that of FIG. 32.

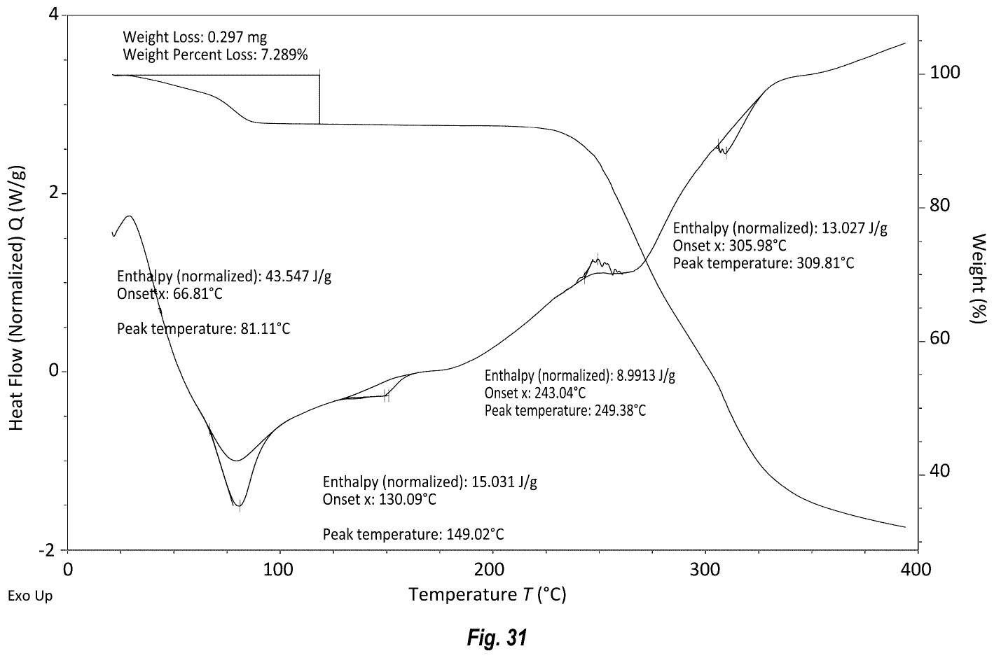

In some embodiments, the Compound 1 hydrogen sulfate or hydrate or solvate thereof, including crystalline Form 2 Compound 1 di(hydrogen sulfate) trihydrate, exhibits a mass loss of between about 5% wt and about 9% wt upon heating from about 20° C. to about 130° C. In some embodiments, the Compound 1 hydrogen sulfate or hydrate or solvate thereof, including crystalline Form 2 Compound 1 di(hydrogen sulfate) trihydrate, exhibits a weight loss of about 6% wt upon heating from about 20° C. to about 130° C. In some embodiments, the Compound 1 hydrogen sulfate or hydrate or solvate thereof, including crystalline Form 2 Compound 1 di(hydrogen sulfate) trihydrate, exhibits a weight loss of about 7% wt upon heating from about 20° C. to about 130° C. In some embodiments, the Compound 1 hydrogen sulfate or hydrate or solvate thereof, including crystalline Form 2 Compound 1 di(hydrogen sulfate) trihydrate, has a TGA thermogram substantially the same as in FIG. 31.

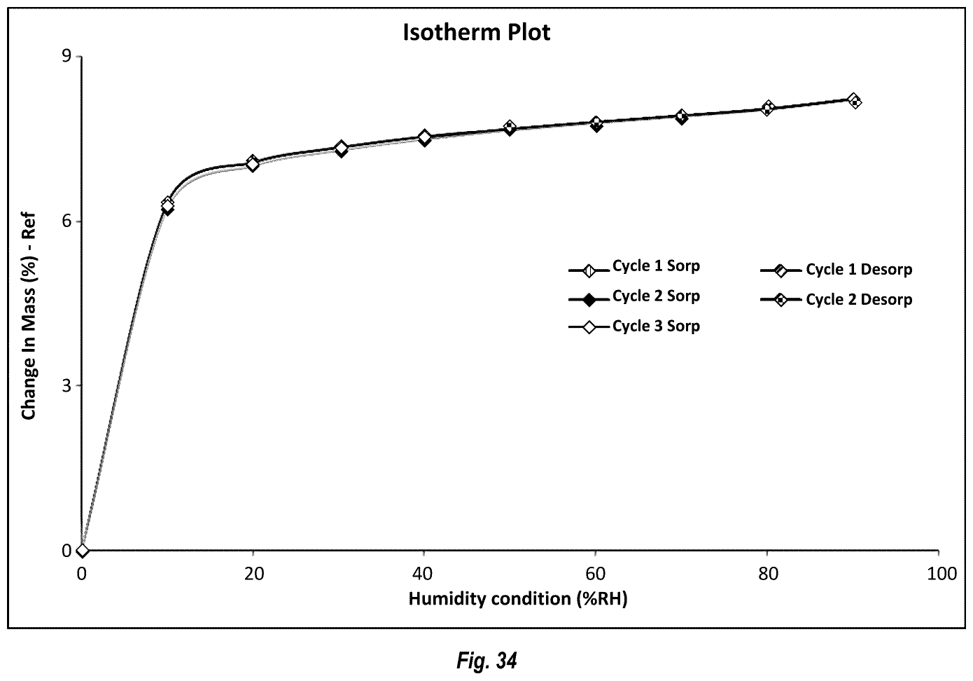

In some embodiments, the Compound 1 hydrogen sulfate or hydrate or solvate thereof, including crystalline Form 2 Compound 1 di(hydrogen sulfate) trihydrate, exhibits a moisture uptake of between about 5% and about 7% between about 0% RH and about 10% RH. In some embodiments, the moisture uptake is between about 6% and about 6.5% between about 0% RH and about 10% RH. In some embodiments, the moisture uptake is about 6% between about 0% RH and about 10% RH. In some embodiments, the moisture uptake is about 7% between about 0% RH and about 10% RH. In some embodiments, the Compound 1 hydrogen sulfate or hydrate or solvate thereof, including crystalline Form 2 Compound 1 di(hydrogen sulfate) trihydrate, has an isotherm plot substantially the same as in FIG. 34.

In some embodiments, the Compound 1 hydrogen sulfate or hydrate or solvate thereof, including crystalline Form 2 Compound 1 di(hydrogen sulfate) trihydrate, has a water content of between about 4% and about 7%. In some embodiments, the water content is between about 6% and about 7%. In some embodiments, the water content is about 4%. In some embodiments, the water content is about 6%. In some embodiments, the water content is about 7%.

In some embodiments, the Compound 1 hydrogen sulfate or hydrate or solvate thereof, including crystalline Form 2 Compound 1 di(hydrogen sulfate) trihydrate, has a chemical purity of about 95%. In some embodiments, the chemical purity is about 97%. In some embodiments, the chemical purity is 99%. In some embodiments, the chemical purity is greater than 99%.

In one aspect, the present disclosure relates to crystalline Form 2 Compound 1 di(hydrogen sulfate) trihydrate. Crystalline Form 2 Compound 1 di(hydrogen sulfate) trihydrate is currently being investigated in patients with advanced solid tumors. Crystalline Form 2 Compound 1 di(hydrogen sulfate) trihydrate has a higher melting point than the corresponding crystalline free base, which is useful when manufacturing a drug product. Further, Applicants discovered that crystalline Form 2 Compound 1 (dihydrogen sulfate) trihydrate often crystallizes into a plate-like crystal habit which may be advantageous in manufacturing tablets, for example. In addition, Applicant surprisingly found that Compound 1 hydrogen sulfate and hydrates and solvates thereof were superior to other multicomponent complexes between Compound 1 and other acids based on, for example, issues with reproducibility, crystallinity, form stability, and higher hygroscopicity.

In some embodiments, the crystalline Form 2 Compound 1 di(hydrogen sulfate) trihydrate has an X-ray powder diffraction pattern comprising one or more peaks chosen from the peak list in Table 2 below (all peaks that can be or have been chosen from Table 2 are rounded to the nearest 0.1° 2θ). In some embodiments, the crystalline Form 2 Compound 1 di(hydrogen sulfate) trihydrate has an X-ray powder diffraction pattern comprising two or more peaks chosen from the peak list in Table 2 below (all peaks that can be or have been chosen from Table 2 are rounded to the nearest 0.1° 2θ). In some embodiments, the crystalline Form 2 Compound 1 di(hydrogen sulfate) trihydrate has an X-ray powder diffraction pattern comprising three or more peaks chosen from the peak list in Table 2 below (all peaks that can be or have been chosen from Table 2 are rounded to the nearest 0.1° 2θ).

TABLE 2
Peak list for crystalline Form 2 Compound
1 di(hydrogen sulfate) trihydrate

| No. | Pos. [°2θ] | d-spacing [Å] | Height [cts] | Rel. Int. [%] |
|---|---|---|---|---|
| 1 | 3.2145 | 27.4858 | 144.12 | 16.34 |
| 2 | 4.8584 | 18.18883 | 503.65 | 57.11 |
| 3 | 9.8661 | 8.96524 | 282.03 | 31.98 |
| 4 | 10.2164 | 8.65865 | 784.06 | 88.9 |
| 5 | 11.6994 | 7.56419 | 119.98 | 13.6 |
| 6 | 12.6578 | 6.99355 | 140.57 | 15.94 |
| 7 | 14.3558 | 6.16994 | 159.37 | 18.07 |
| 8 | 15.0008 | 5.90607 | 803.41 | 91.1 |
| 9 | 15.6918 | 5.64753 | 424.18 | 48.1 |
| 10 | 16.2234 | 5.46363 | 127.56 | 14.46 |
| 11 | 18.0641 | 4.91085 | 105.13 | 11.92 |
| 12 | 19.0052 | 4.66973 | 225.18 | 25.53 |
| 13 | 19.5999 | 4.52935 | 237.59 | 26.94 |
| 14 | 20.019 | 4.43548 | 142.38 | 16.14 |
| 15 | 20.3701 | 4.35981 | 881.93 | 100 |
| 16 | 21.4304 | 4.14645 | 352.47 | 39.97 |
| 17 | 21.7663 | 4.08321 | 585.79 | 66.42 |
| 18 | 22.0553 | 4.03035 | 135.37 | 15.35 |
| 19 | 22.6211 | 3.93081 | 160.73 | 18.23 |
| 20 | 23.3558 | 3.8088 | 120.51 | 13.66 |
| 21 | 23.7449 | 3.74726 | 454.73 | 51.56 |
| 22 | 24.2988 | 3.66308 | 159.23 | 18.05 |
| 23 | 25.0687 | 3.55229 | 188.58 | 21.38 |
| 24 | 25.3332 | 3.51581 | 306.82 | 34.79 |
| 25 | 25.5499 | 3.48647 | 253.82 | 28.78 |
| 26 | 26.2676 | 3.39282 | 56.63 | 6.42 |
| 27 | 26.5834 | 3.35323 | 95.33 | 10.81 |
| 28 | 27.4053 | 3.2545 | 94.49 | 10.71 |
| 29 | 29.7707 | 3.00109 | 50.92 | 5.77 |
| 30 | 33.0781 | 2.70819 | 33 | 3.74 |

Below is the peak list and parameters used for a simulated XRPD diffractogram that was calculated for crystalline Form 2 Compound 1 di(hydrogen sulfate) trihydrate from the crystallographic analysis conducted at 295 K. Accordingly, in some embodiments, the Compound 1 hydrogen sulfate or a hydrate or solvate thereof, including crystalline Form 2 Compound 1 di(hydrogen sulfate) trihydrate, has an X-ray powder diffraction pattern comprising a peak at about 4.9° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ and about 9.9° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ and about 11.8° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ and about 15.1° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ, about 11.8° 2θ, and about 15.1° 2θ.

In some embodiments, the Compound 1 hydrogen sulfate or hydrate or solvate thereof, including crystalline Form 2 Compound 1 di(hydrogen sulfate) trihydrate, has an X-ray powder diffraction pattern comprising one or more peaks chosen from about 4.9° 2θ, about 9.9° 2θ, about 10.3° 2θ, about 11.8° 2θ, about 12.7° 2θ, about 14.4° 2θ, about 15.1° 2θ, about 15.7° 2θ, about 19.1° 2θ, and about 19.7° 2θ.

In some embodiments, the Compound 1 hydrogen sulfate or hydrate or solvate thereof, including crystalline Form 2 Compound 1 di(hydrogen sulfate) trihydrate, has an X-ray powder diffraction pattern comprising a peak at about 4.9° 2θ and one or more peaks chosen from peaks at about 10.3° 2θ and about 15.1° 2θ.

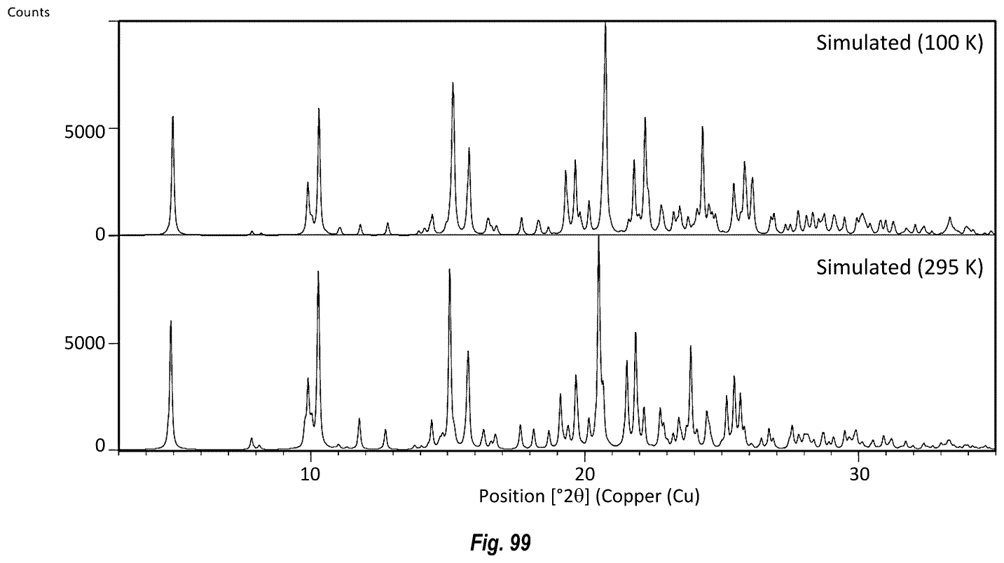

In some embodiments, the Compound 1 hydrogen sulfate or hydrate or solvate, including crystalline Form 2 Compound 1 di(hydrogen sulfate) trihydrate, has an X-ray powder diffraction pattern substantially the same as the bottom pattern of FIG. 99 (Simulated at 295 K).

In some embodiments, the crystalline Form 2 Compound 1 di(hydrogen sulfate) trihydrate has an X-ray powder diffraction pattern comprising one or more peaks chosen from the peak list in Table 4 or 5 below (all peaks that can be or have been chosen from Table 4 or 5 are rounded to the nearest 0.1° 2θ). In some embodiments, the crystalline Form 2 Compound 1 di(hydrogen sulfate) trihydrate has an X-ray powder diffraction pattern comprising two or more peaks chosen from the peak list in Table 4 or 5 below (all peaks that can be or have been chosen from Table 4 or 5 are rounded to the nearest 0.1° 2θ). In some embodiments, the crystalline Form 2 Compound 1 di(hydrogen sulfate) trihydrate has an X-ray powder diffraction pattern comprising three or more peaks chosen from the peak list in Table 4 or 5 below (all peaks that can be or have been chosen from Table 4 or 5 are rounded to the nearest 0.1° 2θ).

TABLE 3
Crystallographic parameters and refinement indicators of Crystalline
Form 2 Compound 1 di(hydrogen sulfate) trihydrate (295 K)

| Empirical | C₃₃H₅₅N₃O₁₉S₂ |
|---|---|
| formula | *the calculated formula differs from the reported formula by two |
| | hydrogen atoms and one oxygen atom that were not located |
| Formula weight | 858.89 g/mol |
| Temperature/K | 295 |
| Crystal system | Triclinic |
| Space group | P1 |
| a/Å | 9.669(2) |
| b/Å | 12.439(3) |
| c/Å | 18.717(4) |
| α/° | 102.143(13) |
| β/° | 94.505(14) |
| γ/° | 110.746(12) |
| Volume/Å³ | 2029.0(8) |
| Z, Z′ | 2, 2 |
| ρ_{calc} g/cm³ | 1.406 |
| μ/mm⁻¹ | 1.896 |
| F(000) | 910.0 |
| Crystal size/mm³ | 0.26 × 0.18 × 0.1 |
| Radiation | CuKα (λ = 1.54184 Å) |
| 2Θ range for data | 4.898 to 133.718 |
| collection/° | |
| Index ranges | −11 ≤ h ≤ 11, −14 ≤ k ≤ 14, −22 ≤ 1 ≤ 22 |
| Reflections | 59429 |
| collected | |
| Independent | 13898 [Rᵢₙₜ = 0.0790, R_{sigma} = 0.0782] |
| reflections | |
| Data/restraints/ | 13898/9/1037 |
| parameters | |
| S | 1.141 |
| Final R indexes | R₁ = 0.0835, wR₂ = 0.2430 |
| [F² > 2σ (F²)] | |
| Final R indexes | R₁ = 0.1304, wR₂ = 0.2767 |
| [all data] | |
| Δρmax, Δρmin/ | 0.75/−0.72 |
| e Å⁻³ | |
| Flack parameter | 0.016(16) |

R₁ = (Σ |Fₒ| − |F_{c}|)/Σ |Fₒ|); wR₂ = {Σ [w(Fₒ² − F_{c}²)²]/Σ [w(Fₒ²)²]}^{1/2}; S = {Σ [w(Fₒ² − F_{c}²)²]/(n−p)}^{1/2.}

TABLE 4
Simulated XRPD 2θ diffractogram of Crystalline Form 2 Compound 1
di(hydrogen sulfate) trihydrate (295 K) (to 35° 2θ).

| No. | Pos. [°2θ] | d-spacing [Å] | Height [cts] | Rel. Int. [%] |
|---|---|---|---|---|
| 1 | 4.8946 | 18.03960 | 6042.30 | 59.50 |
| 2 | 7.8547 | 11.24670 | 556.77 | 5.48 |
| 3 | 8.1269 | 10.87055 | 210.73 | 2.08 |
| 4 | 9.9142 | 8.91452 | 3372.58 | 33.21 |
| 5 | 10.2843 | 8.59445 | 8325.80 | 81.99 |
| 6 | 11.0213 | 8.02136 | 246.99 | 2.43 |
| 7 | 11.3260 | 7.80626 | 124.83 | 1.23 |
| 8 | 11.7769 | 7.50836 | 1449.36 | 14.27 |
| 9 | 12.7288 | 6.94891 | 946.25 | 9.32 |
| 10 | 13.7978 | 6.41286 | 208.45 | 2.05 |
| 11 | 14.0557 | 6.29576 | 175.68 | 1.73 |
| 12 | 14.4212 | 6.13703 | 1381.48 | 13.60 |
| 13 | 14.8161 | 5.97431 | 798.24 | 7.86 |
| 14 | 15.0866 | 5.86780 | 8503.98 | 83.74 |
| 15 | 15.7472 | 5.62311 | 4635.76 | 45.65 |
| 16 | 16.3067 | 5.43141 | 950.60 | 9.36 |
| 17 | 16.5804 | 5.34236 | 396.22 | 3.90 |
| 18 | 16.7518 | 5.28810 | 732.42 | 7.21 |
| 19 | 17.6648 | 5.01677 | 1162.56 | 11.45 |
| 20 | 18.1455 | 4.88495 | 977.39 | 9.63 |
| 21 | 18.7007 | 4.74114 | 895.86 | 8.82 |
| 22 | 19.1179 | 4.63861 | 2593.46 | 25.54 |
| 23 | 19.3951 | 4.57295 | 1135.31 | 11.18 |
| 24 | 19.6844 | 4.50638 | 3525.96 | 34.72 |
| 25 | 20.1541 | 4.40241 | 1482.37 | 14.60 |
| 26 | 20.5087 | 4.32709 | 10154.64 | 100.00 |
| 27 | 20.6883 | 4.28991 | 3063.49 | 30.17 |
| 28 | 21.5353 | 4.12307 | 4158.59 | 40.95 |
| 29 | 21.8691 | 4.06089 | 5594.20 | 55.09 |
| 30 | 22.1724 | 4.00602 | 2019.79 | 19.89 |
| 31 | 22.7572 | 3.90438 | 1932.14 | 19.03 |
| 32 | 23.2319 | 3.82567 | 774.16 | 7.62 |
| 33 | 23.4414 | 3.79193 | 1503.00 | 14.80 |
| 34 | 23.8808 | 3.72316 | 4836.63 | 47.63 |
| 35 | 24.0983 | 3.69005 | 976.79 | 9.62 |
| 36 | 24.4603 | 3.63624 | 1794.58 | 17.67 |
| 37 | 25.1790 | 3.53406 | 2515.82 | 24.78 |
| 38 | 25.4667 | 3.49479 | 3482.27 | 34.29 |
| 39 | 25.6807 | 3.46614 | 2622.15 | 25.82 |
| 40 | 25.8405 | 3.44507 | 1042.79 | 10.27 |
| 41 | 26.0935 | 3.41223 | 282.03 | 2.78 |
| 42 | 26.4560 | 3.36630 | 534.53 | 5.26 |
| 43 | 26.7225 | 3.33333 | 988.58 | 9.74 |
| 44 | 26.8890 | 3.31307 | 537.29 | 5.29 |
| 45 | 27.5852 | 3.23101 | 1124.78 | 11.08 |
| 46 | 27.8048 | 3.20599 | 721.02 | 7.10 |
| 47 | 28.0213 | 3.18171 | 737.19 | 7.26 |
| 48 | 28.1626 | 3.16606 | 700.32 | 6.90 |
| 49 | 28.3719 | 3.14318 | 450.70 | 4.44 |
| 50 | 28.7104 | 3.10689 | 799.44 | 7.87 |
| 51 | 28.9146 | 3.08541 | 359.05 | 3.54 |
| 52 | 29.0906 | 3.06714 | 581.41 | 5.73 |
| 53 | 29.4930 | 3.02620 | 888.83 | 8.75 |
| 54 | 29.6586 | 3.00969 | 595.93 | 5.87 |
| 55 | 29.9076 | 2.98519 | 897.31 | 8.84 |
| 56 | 30.1441 | 2.96231 | 360.12 | 3.55 |
| 57 | 30.5331 | 2.92544 | 448.44 | 4.42 |
| 58 | 30.9066 | 2.89094 | 637.94 | 6.28 |
| 59 | 31.2138 | 2.86318 | 500.03 | 4.92 |
| 60 | 31.7259 | 2.81813 | 403.18 | 3.97 |
| 61 | 31.9896 | 2.79550 | 180.41 | 1.78 |
| 62 | 32.3740 | 2.76318 | 292.01 | 2.88 |
| 63 | 32.7117 | 2.73542 | 173.43 | 1.71 |
| 64 | 32.9971 | 2.71240 | 310.93 | 3.06 |
| 65 | 33.3364 | 2.68557 | 441.08 | 4.34 |
| 66 | 33.9267 | 2.64018 | 201.06 | 1.98 |
| 67 | 34.1530 | 2.62320 | 165.81 | 1.63 |
| 68 | 34.6331 | 2.58793 | 175.24 | 1.73 |

TABLE 5
Simulated XRPD 2θ diffractogram of Crystalline Form 2 Compound
1 di(hydrogen sulfate) trihydrate (295 K) (20 most intense peaks).

| No. | Pos. [°2θ] | d-spacing [Å] | Height [cts] | Rel. Int. [%] |
|---|---|---|---|---|
| 1 | 20.5087 | 4.32709 | 10154.64 | 100.00 |
| 2 | 15.0866 | 5.86780 | 8503.98 | 83.74 |
| 3 | 10.2843 | 8.59445 | 8325.80 | 81.99 |
| 4 | 4.8946 | 18.03960 | 6042.30 | 59.50 |
| 5 | 21.8691 | 4.06089 | 5594.20 | 55.09 |
| 6 | 23.8808 | 3.72316 | 4836.63 | 47.63 |
| 7 | 15.7472 | 5.62311 | 4635.76 | 45.65 |
| 8 | 21.5353 | 4.12307 | 4158.59 | 40.95 |
| 9 | 19.6844 | 4.50638 | 3525.96 | 34.72 |
| 10 | 25.4667 | 3.49479 | 3482.27 | 34.29 |
| 11 | 9.9142 | 8.91452 | 3372.58 | 33.21 |
| 12 | 20.6883 | 4.28991 | 3063.49 | 30.17 |
| 13 | 25.6807 | 3.46614 | 2622.15 | 25.82 |
| 14 | 19.1179 | 4.63861 | 2593.46 | 25.54 |
| 15 | 25.1790 | 3.53406 | 2515.82 | 24.78 |
| 16 | 22.1724 | 4.00602 | 2019.79 | 19.89 |
| 17 | 22.7572 | 3.90438 | 1932.14 | 19.03 |
| 18 | 24.4603 | 3.63624 | 1794.58 | 17.67 |
| 19 | 23.4414 | 3.79193 | 1503.00 | 14.80 |
| 20 | 20.1541 | 4.40241 | 1482.37 | 14.60 |

Below is the peak list and parameters used for a simulated XRPD diffractogram that was calculated for crystalline Form 2 Compound 1 di(hydrogen sulfate) trihydrate at 100 K. Accordingly, in some embodiments, the crystalline Form 2 Compound 1 di(hydrogen sulfate) trihydrate has an X-ray powder diffraction pattern comprising one or more peaks chosen from the peak list in Table 7 or 8 below (all peaks that can be or have been chosen from Table 7 or 8 are rounded to the nearest 0.1° 2θ). In some embodiments, the crystalline Form 2 Compound 1 di(hydrogen sulfate) trihydrate has an X-ray powder diffraction pattern comprising two or more peaks chosen from the peak list in Table 7 or 8 below (all peaks that can be or have been chosen from Table 7 or 8 are rounded to the nearest 0.1° 2θ). In some embodiments, the crystalline Form 2 Compound 1 di(hydrogen sulfate) trihydrate has an X-ray powder diffraction pattern comprising three or more peaks chosen from the peak list in Table 7 or 8 below (all peaks that can be or have been chosen from Table 7 or 8 are rounded to the nearest 0.1° 2θ). In some embodiments, the Compound 1 hydrogen sulfate or hydrate or solvate, including crystalline Form 2 Compound 1 di(hydrogen sulfate) trihydrate, has an X-ray powder diffraction pattern substantially the same as the top pattern of FIG. 99 (Simulated at 100 K).

TABLE 6
Crystallographic parameters and refinement indicators of Crystalline
Form 2 Compound 1 di(hydrogen sulfate) trihydrate (100 K)

| Empirical formula | C₃₃H₅₇N₃O₂₀S₂ |
|---|---|
| Formula weight | 879.93 g/mol |
| Temperature/K | 100 |
| Crystal system | Triclinic |
| Space group | P1 |
| a/Å | 9.6794(2) |
| b/Å | 12.3886(2) |
| c/Å | 18.4016(4) |
| α/° | 101.6708(8) |
| β/° | 94.7448(8) |
| γ/° | 110.7514(8) |
| Volume/Å³ | 1991.88(7) |
| Z, Z′ | 2, 2 |
| ρ_{calc} g/cm³ | 1.467 |
| μ/mm⁻¹ | 1.962 |
| F(000) | 936.0 |
| Crystal size/mm³ | 0.38 × 0.24 × 0.12 |
| Radiation | CuKα (λ = 1.54178 Å) |
| 2Θ range for data collection/° | 4.974 to 144.29 |
| Index ranges | −10 ≤ h ≤ 11, −15 < k ≤ 15, −22 ≤ 1 ≤ 2 |
| Reflections collected | 97841 |
| Independent reflections | 15198 [Rᵢₙₜ = 0.0299, R_{sigma} = 0.0202] |
| Data/restraints/parameters | 15198/33/1139 |
| S | 1.029 |
| Final R indexes [F² > 2σ (F²)] | R₁ = 0.0586, wR₂ = 0.1588 |
| Final R indexes [all data] | R₁ = 0.0592, wR₂ = 0.1597 |
| Δρmax, Δρmin/e Å⁻³ | 0.63/−1.17 |
| Flack parameter | −0.008(5) |

R₁ = (Σ |Fₒ| − |F_{c}|)/Σ |Fₒ|); wR₂ = {Σ [w(Fₒ² − F_{c}²)²]/Ε [w(Fₒ²)²]}^{1/2}; S = {Σ [w(Fₒ² − F_{c}²)²]/(n−p)}^{1/2.}

TABLE 7
Simulated XRPD 2θ diffractogram of Crystalline Form 2 Compound 1
di(hydrogen sulfate) trihydrate (100 K) (to 35° 2θ).

| No. | Pos. [°2θ] | d-spacing [Å] | Height [cts] | Rel. Int. [%] |
|---|---|---|---|---|
| 1 | 4.9708 | 17.76340 | 5692.39 | 57.05 |
| 2 | 7.8721 | 11.22177 | 192.87 | 1.93 |
| 3 | 8.2012 | 10.77221 | 118.89 | 1.19 |
| 4 | 9.9051 | 8.92269 | 2513.16 | 25.19 |
| 5 | 10.3093 | 8.57375 | 6074.58 | 60.88 |
| 6 | 11.0697 | 7.98639 | 381.20 | 3.82 |
| 7 | 11.8171 | 7.48292 | 506.02 | 5.07 |
| 8 | 12.8134 | 6.90323 | 597.90 | 5.99 |
| 9 | 13.9479 | 6.34418 | 211.75 | 2.12 |
| 10 | 14.1586 | 6.25023 | 346.39 | 3.47 |
| 11 | 14.4510 | 6.12442 | 967.78 | 9.70 |
| 12 | 15.2031 | 5.82311 | 7129.03 | 71.45 |
| 13 | 15.7809 | 5.61116 | 4079.65 | 40.89 |
| 14 | 16.4745 | 5.37646 | 794.87 | 7.97 |
| 15 | 16.7852 | 5.27763 | 453.42 | 4.54 |
| 16 | 17.7061 | 5.00516 | 847.97 | 8.50 |
| 17 | 18.3200 | 4.83881 | 709.84 | 7.11 |
| 18 | 18.6841 | 4.74533 | 393.29 | 3.94 |
| 19 | 19.3213 | 4.59023 | 3032.46 | 30.39 |
| 20 | 19.6644 | 4.51092 | 3526.60 | 35.34 |
| 21 | 19.8463 | 4.46998 | 1072.98 | 10.75 |
| 22 | 20.1614 | 4.40082 | 1610.14 | 16.14 |
| 23 | 20.7696 | 4.27331 | 9978.21 | 100.00 |
| 24 | 21.5995 | 4.11096 | 759.84 | 7.61 |
| 25 | 21.8102 | 4.07171 | 3594.54 | 36.02 |
| 26 | 22.2082 | 3.99964 | 5614.95 | 56.27 |
| 27 | 22.3481 | 3.97491 | 1892.70 | 18.97 |
| 28 | 22.7901 | 3.89882 | 1412.77 | 14.16 |
| 29 | 23.2451 | 3.82351 | 1120.21 | 11.23 |
| 30 | 23.4845 | 3.78508 | 1365.48 | 13.68 |
| 31 | 23.7730 | 3.73979 | 884.41 | 8.86 |
| 32 | 24.0898 | 3.69132 | 1251.47 | 12.54 |
| 33 | 24.3072 | 3.65880 | 5161.43 | 51.73 |
| 34 | 24.5353 | 3.62530 | 1465.70 | 14.69 |
| 35 | 24.7824 | 3.58971 | 996.51 | 9.99 |
| 36 | 25.4465 | 3.49751 | 2461.31 | 24.67 |
| 37 | 25.8398 | 3.44517 | 3425.07 | 34.33 |
| 38 | 26.1214 | 3.40866 | 2704.76 | 27.11 |
| 39 | 26.7881 | 3.32531 | 828.92 | 8.31 |
| 40 | 26.9212 | 3.30917 | 1036.14 | 10.38 |
| 41 | 27.3278 | 3.26086 | 516.98 | 5.18 |
| 42 | 27.5109 | 3.23957 | 527.00 | 5.28 |
| 43 | 27.7881 | 3.20787 | 1169.38 | 11.72 |
| 44 | 28.1012 | 3.17284 | 937.34 | 9.39 |
| 45 | 28.3241 | 3.14838 | 1081.95 | 10.84 |
| 46 | 28.5401 | 3.12505 | 773.11 | 7.75 |
| 47 | 28.7454 | 3.10318 | 1024.38 | 10.27 |
| 48 | 29.1078 | 3.06537 | 964.03 | 9.66 |
| 49 | 29.4874 | 3.02676 | 857.33 | 8.59 |
| 50 | 29.9255 | 2.98345 | 806.76 | 8.09 |
| 51 | 30.1457 | 2.96215 | 1038.18 | 10.40 |
| 52 | 30.4145 | 2.93659 | 577.92 | 5.79 |
| 53 | 30.7961 | 2.90106 | 714.10 | 7.16 |
| 54 | 30.9877 | 2.88356 | 724.52 | 7.26 |
| 55 | 31.2722 | 2.85797 | 663.29 | 6.65 |
| 56 | 31.7460 | 2.81639 | 339.03 | 3.40 |
| 57 | 32.0668 | 2.78894 | 523.95 | 5.25 |
| 58 | 32.3819 | 2.76252 | 429.15 | 4.30 |
| 59 | 32.6774 | 2.73821 | 209.40 | 2.10 |
| 60 | 33.3433 | 2.68503 | 859.27 | 8.61 |
| 61 | 33.9170 | 2.64091 | 408.11 | 4.09 |
| 62 | 34.1856 | 2.62078 | 272.79 | 2.73 |
| 63 | 34.6088 | 2.58969 | 110.44 | 1.11 |
| 64 | 34.8394 | 2.57308 | 228.50 | 2.29 |

TABLE 8
Simulated XRPD 2θ diffractogram of Crystalline Form 2 Compound
1 di(hydrogen sulfate) trihydrate (100K) (20 most intense peaks).

| No. | Pos. [° 2θ] | d-spacing [Å] | Height [cts] | Rel. Int. [%] |
|---|---|---|---|---|
| 1 | 20.7696 | 4.27331 | 9978.21 | 100.00 |
| 2 | 15.2031 | 5.82311 | 7129.03 | 71.45 |
| 3 | 10.3093 | 8.57375 | 6074.58 | 60.88 |
| 4 | 4.9708 | 17.76340 | 5692.39 | 57.05 |
| 5 | 22.2082 | 3.99964 | 5614.95 | 56.27 |
| 6 | 24.3072 | 3.65880 | 5161.43 | 51.73 |
| 7 | 15.7809 | 5.61116 | 4079.65 | 40.89 |
| 8 | 21.8102 | 4.07171 | 3594.54 | 36.02 |
| 9 | 19.6644 | 4.51092 | 3526.60 | 35.34 |
| 10 | 25.8398 | 3.44517 | 3425.07 | 34.33 |
| 11 | 19.3213 | 4.59023 | 3032.46 | 30.39 |
| 12 | 26.1214 | 3.40866 | 2704.76 | 27.11 |
| 13 | 9.9051 | 8.92269 | 2513.16 | 25.19 |
| 14 | 25.4465 | 3.49751 | 2461.31 | 24.67 |
| 15 | 22.3481 | 3.97491 | 1892.70 | 18.97 |
| 16 | 20.1614 | 4.40082 | 1610.14 | 16.14 |
| 17 | 24.5353 | 3.62530 | 1465.70 | 14.69 |
| 18 | 22.7901 | 3.89882 | 1412.77 | 14.16 |
| 19 | 23.4845 | 3.78508 | 1365.48 | 13.68 |
| 20 | 24.0898 | 3.69132 | 1251.47 | 12.54 |

Crystalline Form 2 Compound 1 di(hydrogen sulfate) trihydrate contains one stereogenic center and is a single enantiomer with the R-configuration, as shown in Table 9. It contains three molecules of water, as shown in Table 9, and thus is a trihydrate.

TABLE 9

| | |
|---|---|
| Chemical Structure | 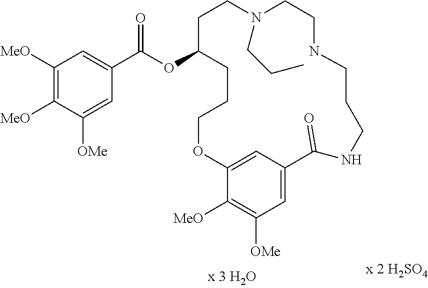 [SMILES: CCCN1CCNCCCNC(=O)c2cc(OC)c(OC)c(c2)OCCC[C@@H](OC(=O)c2cc(OC)c(OC)c(OC)c2)CC1] |
| Molecular formula | C₃₃H₄₇N₃O₉, 2 H₂SO₄, 3 H₂O (C₃₃H₅₁N₃O₁₇S₂, 3 H₂O) |
| Relative molecular | 629.75 (free base); 879.94 (salt: di(hydrogen sulfate)trihydrate) |
| mass (MW) | |

Crystalline Form 2 Compound 1 di(hydrogen sulfate) trihydrate has a single Compound 1 cation and at least one hydrogen sulfate anion and where the other sulfate moiety may be in the form of a second hydrogen sulfate anion or sulfuric acid (which would be neutral).

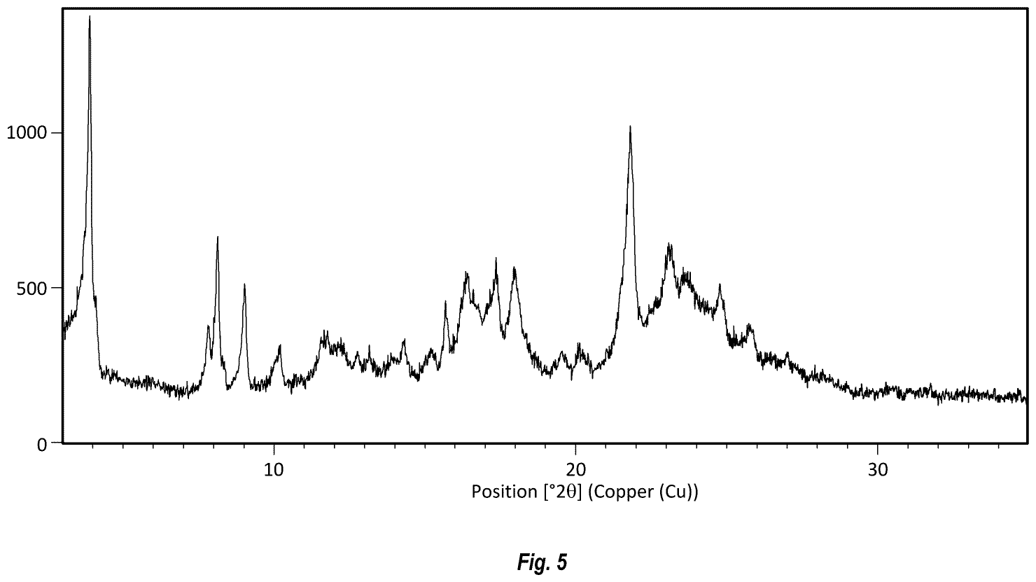

In one aspect, the present disclosure relates to Compound 1 hydrogen sulfate or a hydrate or solvate thereof, including crystalline Form 3 Compound 1 di(hydrogen sulfate) or a hydrate or solvate thereof, having an X-ray powder diffraction pattern comprising a peak at about 3.9° 2θ. In some embodiments, the Compound 1 hydrogen sulfate or hydrate or solvate thereof, including crystalline Form 3 Compound 1 di(hydrogen sulfate) or a hydrate or solvate thereof, has an X-ray powder diffraction pattern comprising one or more peaks chosen from peaks at about 3.9° 2θ, about 7.8° 2θ, about 10.2° 2θ, about 15.7° 2θ, and about 19.5° 2θ. In some embodiments, the Compound 1 hydrogen sulfate or hydrate or solvate thereof, including crystalline Form 3 Compound 1 di(hydrogen sulfate) or a hydrate or solvate thereof, has an X-ray powder diffraction pattern comprising peaks at about 3.9° 2θ, about 7.8° 2θ, about 10.2° 2θ, about 15.7° 2θ, and about 19.5° 2θ. In some embodiments, the Compound 1 hydrogen sulfate or hydrate or solvate thereof, including crystalline Form 3 Compound 1 di(hydrogen sulfate) or a hydrate or solvate thereof, has an X-ray powder diffraction pattern substantially the same as that of FIG. 5.

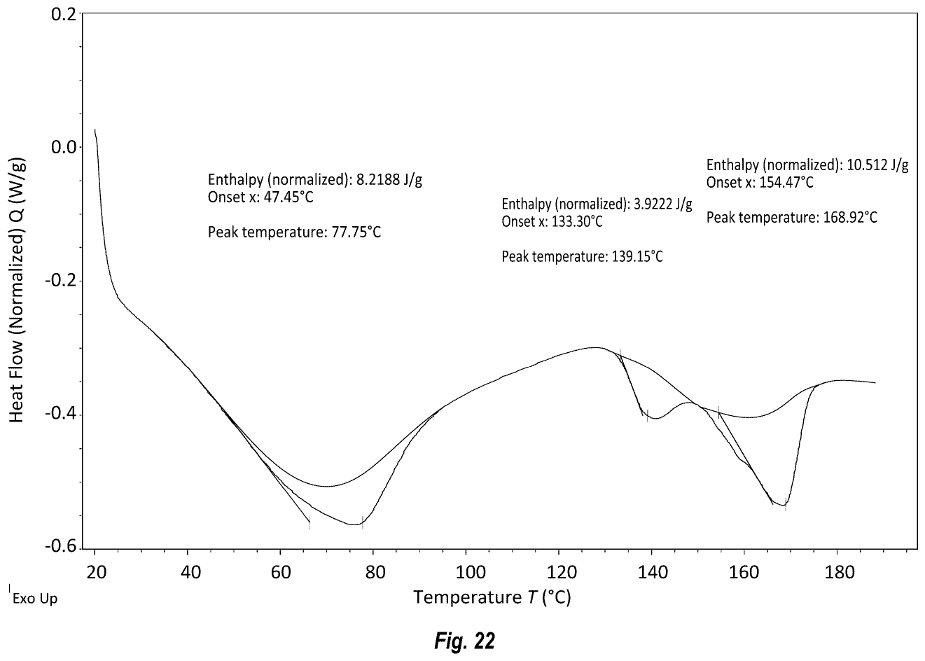

In some embodiments, the Compound 1 hydrogen sulfate or hydrate or solvate thereof, including crystalline Form 3 Compound 1 di(hydrogen sulfate) or a hydrate or solvate thereof, has an FT-IR spectrum substantially the same as that of FIG. 23B. In some embodiments, the Compound 1 hydrogen sulfate or hydrate or solvate thereof, including crystalline Form 3 Compound 1 di(hydrogen sulfate) or a hydrate or solvate thereof, has a melting onset as measured by DSC in an open aluminum pan of about 163° C. In some embodiments, the Compound 1 hydrogen sulfate or hydrate or solvate thereof, including crystalline Form 3 Compound 1 di(hydrogen sulfate) or a hydrate or solvate thereof, has a DSC thermogram substantially the same as that of FIG. 22.

In one aspect, the present disclosure relates to crystalline Form 3 Compound 1 di(hydrogen sulfate) or a hydrate or solvate thereof. In some embodiments, the crystalline Form 3 Compound 1 di(hydrogen sulfate) or hydrate or solvate thereof is a hydrate. In some embodiments, the crystalline Form 3 Compound 1 di(hydrogen sulfate) or hydrate thereof is a monohydrate.

In some embodiments, the crystalline Form 3 Compound 1 di(hydrogen sulfate) or hydrate or solvate thereof has an X-ray powder diffraction pattern comprising one or more peaks chosen from the peak list in Table 10 below (all peaks that can be or have been chosen from Table 10 are rounded to the nearest 0.1° 2θ). In some embodiments, the crystalline Form 3 Compound 1 di(hydrogen sulfate) or hydrate or solvate thereof has an X-ray powder diffraction pattern comprising two or more peaks chosen from the peak list in Table 10 below (all peaks that can be or have been chosen from Table 10 are rounded to the nearest 0.1° 2θ). In some embodiments, the crystalline Form 3 Compound 1 di(hydrogen sulfate) or hydrate or solvate thereof has an X-ray powder diffraction pattern comprising three or more peaks chosen from the peak list in Table 10 below (all peaks that can be or have been chosen from Table 10 are rounded to the nearest 0.1° 2θ).

TABLE 10
Peak list for crystalline Form 3 Compound 1 di(hydrogen sulfate)

| No. | Pos. [° 2θ] | d-spacing [Å] | Height [cts] | Rel. Int. [%] |
|---|---|---|---|---|
| 1 | 3.9044 | 22.63109 | 1207.05 | 100 |
| 2 | 7.8271 | 11.29553 | 211.78 | 17.55 |
| 3 | 8.1384 | 10.86418 | 505.84 | 41.91 |
| 4 | 9.0415 | 9.781 | 325.62 | 26.98 |
| 5 | 10.1705 | 8.69757 | 133.39 | 11.05 |
| 6 | 11.5853 | 7.63845 | 155.12 | 12.85 |
| 7 | 14.317 | 6.18656 | 144.54 | 11.97 |
| 8 | 15.6875 | 5.64904 | 258.3 | 21.4 |
| 9 | 16.4236 | 5.39747 | 366.39 | 30.35 |
| 10 | 17.3748 | 5.10408 | 376.88 | 31.22 |
| 11 | 17.972 | 4.93579 | 365.98 | 30.32 |
| 12 | 19.519 | 4.54794 | 96.95 | 8.03 |
| 13 | 20.0869 | 4.42065 | 106.72 | 8.84 |
| 14 | 21.8128 | 4.07461 | 822.88 | 68.17 |
| 15 | 23.1132 | 3.84822 | 432.37 | 35.82 |
| 16 | 23.7695 | 3.74344 | 307.13 | 25.44 |
| 17 | 24.8126 | 3.58837 | 295.89 | 24.51 |
| 18 | 25.8044 | 3.45267 | 170.01 | 14.08 |
| 19 | 26.9721 | 3.30579 | 63.54 | 5.26 |

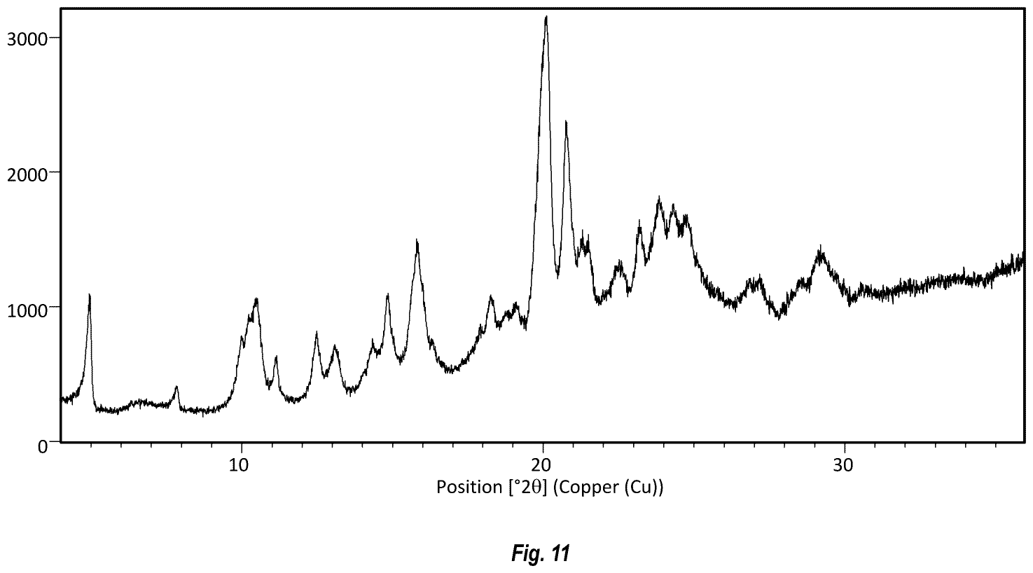

In one aspect, the present disclosure relates to Compound 1 hydrogen sulfate or a hydrate or solvate thereof a hydrate or solvate thereof, including crystalline Form 5 Compound 1 di(hydrogen sulfate), having an X-ray powder diffraction pattern comprising a peak at about 5.0° 2θ. In some embodiments, the Compound 1 hydrogen sulfate or hydrate or solvate thereof, including crystalline Form 5 Compound 1 di(hydrogen sulfate) or a hydrate or solvate thereof, has an X-ray powder diffraction pattern comprising one or more peaks chosen from peaks at about 5.0° 2θ, about 7.8° 2θ, about 10.5° 2θ, and about 11.1° 2θ. In some embodiments, the Compound 1 hydrogen sulfate or hydrate or solvate thereof, including crystalline Form 5 Compound 1 di(hydrogen sulfate) or a hydrate or solvate thereof, has an X-ray powder diffraction pattern comprising a peak at about 5.0° 2θ and one or more peaks chosen from peaks at about 7.8° 2θ, about 10.5° 2θ, and about 11.1° 2θ. In some embodiments, the Compound 1 hydrogen sulfate or hydrate or solvate thereof, including crystalline Form 5 Compound 1 di(hydrogen sulfate) or a hydrate or solvate thereof, has an X-ray powder diffraction pattern comprising peaks at about 5.0° 2θ, about 7.8° 2θ, about 10.5° 2θ, and about 11.1° 2θ. In some embodiments, the Compound 1 hydrogen sulfate or hydrate or solvate thereof, including crystalline Form 5 Compound 1 di(hydrogen sulfate) or a hydrate or solvate thereof, has an X-ray powder diffraction pattern substantially the same as that of FIG. 11.

In one aspect, the present disclosure relates to crystalline Form 5 Compound 1 di(hydrogen sulfate) or a hydrate or solvate thereof. In some embodiments, the crystalline Form 5 Compound 1 di(hydrogen sulfate) or hydrate or solvate thereof has an X-ray powder diffraction pattern comprising one or more peaks chosen from the peak list in Table 11 below (all peaks that can be or have been chosen from Table 11 are rounded to the nearest 0.1° 2θ). In some embodiments, the crystalline Form 5 Compound 1 di(hydrogen sulfate) or hydrate or solvate thereof has an X-ray powder diffraction pattern comprising two or more peaks chosen from the peak list in Table 11 below (all peaks that can be or have been chosen from Table 11 are rounded to the nearest 0.1° 2θ). In some embodiments, the crystalline Form 5 Compound 1 di(hydrogen sulfate) or hydrate or solvate thereof has an X-ray powder diffraction pattern comprising three or more peaks chosen from the peak list in Table 11 below (all peaks that can be or have been chosen from Table 11 are rounded to the nearest 0.1° 2θ).

TABLE 11
Peak list for crystalline Form 5 Compound 1 di(hydrogen sulfate)

| No. | Pos. [° 2θ] | d-spacing [Å] | Height [cts] | Rel. Int. [%] |
|---|---|---|---|---|
| 1 | 4.9586 | 17.82176 | 861.12 | 36.72 |
| 2 | 6.5915 | 13.40992 | 62.14 | 2.65 |
| 3 | 7.8629 | 11.24425 | 172.67 | 7.36 |
| 4 | 9.9807 | 8.86253 | 501.64 | 21.39 |
| 5 | 10.5121 | 8.41569 | 774.53 | 33.03 |
| 6 | 11.1438 | 7.94004 | 315.63 | 13.46 |
| 7 | 12.4842 | 7.09037 | 457.38 | 19.5 |
| 8 | 13.0886 | 6.76432 | 332.66 | 14.18 |
| 9 | 14.3338 | 6.17935 | 300.12 | 12.8 |
| 10 | 14.8292 | 5.97401 | 633.76 | 27.02 |
| 11 | 15.8321 | 5.59779 | 977.9 | 41.7 |
| 12 | 18.2938 | 4.8497 | 475.54 | 20.28 |
| 13 | 19.1375 | 4.63775 | 391.89 | 16.71 |
| 14 | 20.1613 | 4.40449 | 2345.22 | 100 |
| 15 | 20.7629 | 4.27822 | 1699.99 | 72.49 |
| 16 | 21.5067 | 4.13191 | 747.6 | 31.88 |
| 17 | 22.4585 | 3.9589 | 550.04 | 23.45 |
| 18 | 23.1731 | 3.83841 | 811.13 | 34.59 |
| 19 | 23.8388 | 3.73271 | 1011.38 | 43.13 |
| 20 | 24.2785 | 3.66609 | 923.17 | 39.36 |
| 21 | 24.712 | 3.60276 | 817.76 | 34.87 |
| 22 | 27.2221 | 3.27599 | 249.23 | 10.63 |
| 23 | 28.4081 | 3.14186 | 158.81 | 6.77 |
| 24 | 29.0742 | 3.07138 | 345.45 | 14.73 |
| 25 | 30.6695 | 2.91516 | 50.04 | 2.13 |

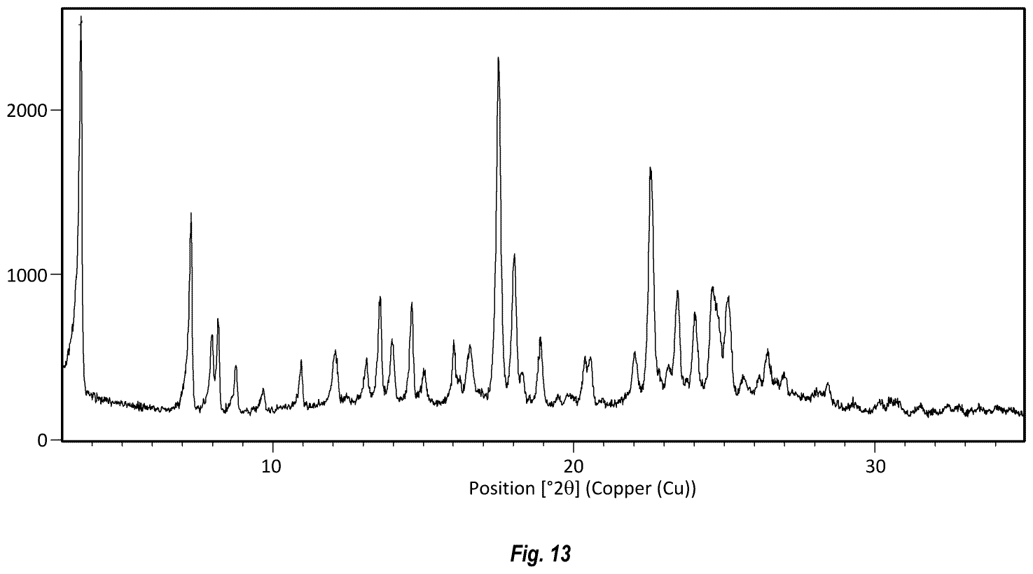

In one aspect, the present disclosure relates to Compound 1 hydrogen sulfate or a hydrate or solvate thereof, including crystalline Form 6 Compound 1 di(hydrogen sulfate) or a hydrate or solvate thereof, having an X-ray powder diffraction pattern comprising a peak at about 3.6° 2θ. In some embodiments, the Compound 1 hydrogen sulfate or hydrate or solvate thereof, including crystalline Form 6 Compound 1 di(hydrogen sulfate) or a hydrate or solvate thereof, has an X-ray powder diffraction pattern comprising one or more peaks chosen from peaks at about 3.6° 2θ, about 7.3° 2θ, about 13.1° 2θ, and about 14.6° 2θ. In some embodiments, the Compound 1 hydrogen sulfate or hydrate or solvate thereof, including crystalline Form 6 Compound 1 di(hydrogen sulfate) or a hydrate or solvate thereof, has an X-ray powder diffraction pattern comprising peaks at about 3.6° 2θ, about 7.3° 2θ, about 13.1° 2θ, and about 14.6° 2θ. In some embodiments, the Compound 1 hydrogen sulfate or hydrate or solvate thereof, including crystalline Form 6 Compound 1 di(hydrogen sulfate) or a hydrate or solvate thereof, has an X-ray powder diffraction pattern substantially the same as that of FIG. 13.

In one aspect, the present disclosure relates to crystalline Form 6 Compound 1 di(hydrogen sulfate) or a hydrate or solvate thereof. In some embodiments, the crystalline Form 6 Compound 1 di(hydrogen sulfate) or hydrate or solvate thereof has an X-ray powder diffraction pattern comprising one or more peaks chosen from the peak list in Table 12 below (all peaks that can be or have been chosen from Table 12 are rounded to the nearest 0.1° 2θ). In some embodiments, the crystalline Form 6 Compound 1 di(hydrogen sulfate) or hydrate or solvate thereof has an X-ray powder diffraction pattern comprising two or more peaks chosen from the peak list in Table 12 below (all peaks that can be or have been chosen from Table 12 are rounded to the nearest 0.1° 2θ). In some embodiments, the crystalline Form 6 Compound 1 di(hydrogen sulfate) or hydrate or solvate thereof has an X-ray powder diffraction pattern comprising three or more peaks chosen from the peak list in Table 12 below (all peaks that can be or have been chosen from Table 12 are rounded to the nearest 0.1° 2θ).

TABLE 12
Peak list for crystalline Form 6 Compound 1 di(hydrogen sulfate)

| No. | Pos. [° 2θ] | d-spacing [Å] | Height [cts] | Rel. Int. [%] |
|---|---|---|---|---|
| 1 | 3.6457 | 24.23649 | 2331.19 | 100 |
| 2 | 7.2964 | 12.11588 | 1219.04 | 52.29 |
| 3 | 7.9727 | 11.08968 | 450.62 | 19.33 |
| 4 | 8.2001 | 10.7826 | 526.8 | 22.6 |
| 5 | 8.7879 | 10.06264 | 272.6 | 11.69 |
| 6 | 9.6919 | 9.12599 | 130.1 | 5.58 |
| 7 | 10.9497 | 8.08032 | 315.45 | 13.53 |
| 8 | 12.0616 | 7.33783 | 356.47 | 15.29 |
| 9 | 13.1127 | 6.75192 | 314.44 | 13.49 |
| 10 | 13.5803 | 6.52046 | 675.85 | 28.99 |
| 11 | 13.9505 | 6.34825 | 421.28 | 18.07 |
| 12 | 14.6229 | 6.05783 | 655.38 | 28.11 |
| 13 | 14.9956 | 5.90811 | 215.48 | 9.24 |
| 14 | 16.0096 | 5.5361 | 447.37 | 19.19 |
| 15 | 16.5706 | 5.34992 | 391.99 | 16.82 |
| 16 | 17.5012 | 5.06749 | 2191.02 | 93.99 |
| 17 | 18.0247 | 4.92149 | 978.53 | 41.98 |
| 18 | 18.2959 | 4.84913 | 235.83 | 10.12 |
| 19 | 18.8901 | 4.69794 | 463.2 | 19.87 |
| 20 | 20.3597 | 4.36202 | 321.19 | 13.78 |
| 21 | 20.5606 | 4.31986 | 331.85 | 14.24 |
| 22 | 22.0077 | 4.03897 | 366.74 | 15.73 |
| 23 | 22.5296 | 3.94331 | 1500.27 | 64.36 |
| 24 | 22.6 | 3.94095 | 1366.54 | 58.62 |
| 25 | 23.1134 | 3.84501 | 266.96 | 11.45 |
| 26 | 23.4488 | 3.79076 | 752.81 | 32.29 |
| 27 | 23.9992 | 3.70506 | 589.32 | 25.28 |
| 28 | 24.5629 | 3.62129 | 696.4 | 29.87 |
| 29 | 25.1055 | 3.54423 | 686.19 | 29.44 |
| 30 | 25.6451 | 3.47087 | 209.07 | 8.97 |
| 31 | 26.4335 | 3.36911 | 370.23 | 15.88 |
| 32 | 27.0285 | 3.29629 | 214.81 | 9.21 |
| 33 | 28.4275 | 3.13717 | 178.34 | 7.65 |
| 34 | 30.6833 | 2.91146 | 58.3 | 2.5 |
| 35 | 31.5194 | 2.83611 | 42.07 | 1.8 |

In one aspect, the present disclosure relates to Compound 1 hydrogen sulfate or a hydrate or solvate thereof, including crystalline Form 7 Compound 1 di(hydrogen sulfate) or a hydrate or solvate thereof, having an X-ray powder diffraction pattern comprising a peak at about 3.6° 2θ. In some embodiments, the Compound 1 hydrogen sulfate or hydrate or solvate thereof, including crystalline Form 7 Compound 1 di(hydrogen sulfate) or a hydrate or solvate thereof, has an X-ray powder diffraction pattern comprising one or more peaks chosen from peaks at about 3.6° 2θ, about 7.1° 2θ, about 9.9° 2θ, and about 12.5° 2θ. In some embodiments, the Compound 1 hydrogen sulfate or hydrate or solvate thereof, including crystalline Form 7 Compound 1 di(hydrogen sulfate) or a hydrate or solvate thereof, has an X-ray powder diffraction pattern comprising peaks at about 3.6° 2θ, about 7.1° 2θ, about 9.9° 2θ, and about 12.5° 2θ. In some embodiments, the Compound 1 hydrogen sulfate or hydrate or solvate thereof, including crystalline Form 7 Compound 1 di(hydrogen sulfate) or a hydrate or solvate thereof, has an X-ray powder diffraction pattern comprising peaks at about 3.6° 2θ, about 7.1° 2θ, about 9.9° 2θ, about 12.5° 2θ, and about 21.5° 2θ.

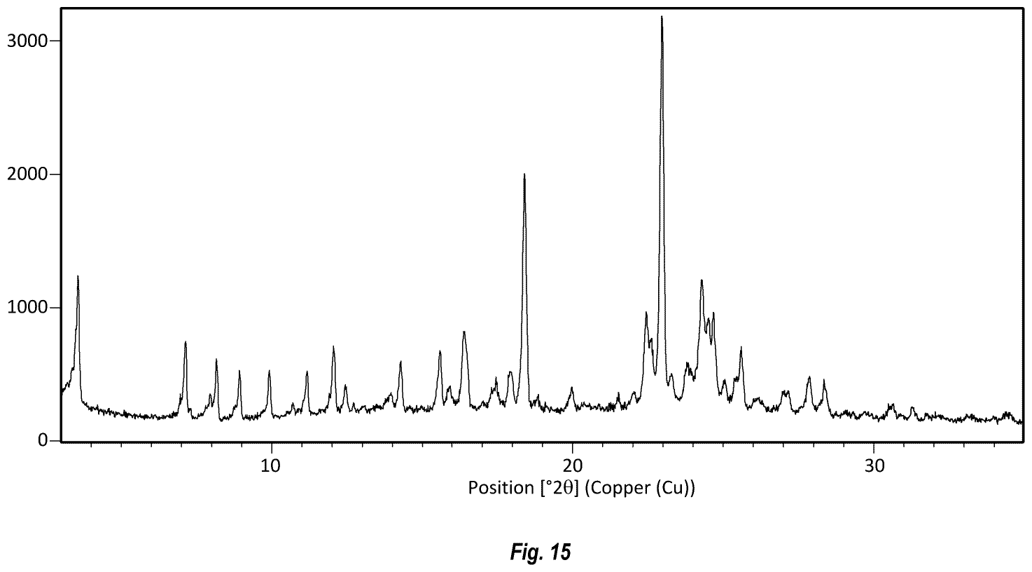

In some embodiments, the Compound 1 hydrogen sulfate or hydrate or solvate thereof, including crystalline Form 7 Compound 1 di(hydrogen sulfate) or a hydrate or solvate thereof, has an X-ray powder diffraction pattern substantially the same as that of FIG. 15.

In one aspect, the present disclosure relates to crystalline Form 7 Compound 1 di(hydrogen sulfate) or a hydrate or solvate thereof. In some embodiments, the crystalline Form 7 Compound 1 di(hydrogen sulfate) or hydrate or solvate thereof has an X-ray powder diffraction pattern comprising one or more peaks chosen from the peak list in Table 13 below (all peaks that can be or have been chosen from Table 13 are rounded to the nearest 0.1° 2θ). In some embodiments, the crystalline Form 7 Compound 1 di(hydrogen sulfate) or hydrate or solvate thereof has an X-ray powder diffraction pattern comprising two or more peaks chosen from the peak list in Table 13 below (all peaks that can be or have been chosen from Table 13 are rounded to the nearest 0.1° 2θ). In some embodiments, the crystalline Form 7 Compound 1 di(hydrogen sulfate) or hydrate or solvate thereof has an X-ray powder diffraction pattern comprising three or more peaks chosen from the peak list in Table 13 below (all peaks that can be or have been chosen from Table 13 are rounded to the nearest 0.1° 2θ).

TABLE 13
Peak list for crystalline Form 7 Compound 1 di(hydrogen sulfate)

| No. | Pos. [° 2θ] | d-spacing [Å] | Height [cts] | Rel. Int. [%] |
|---|---|---|---|---|
| 1 | 3.5699 | 24.75094 | 1055.05 | 34.09 |
| 2 | 7.1442 | 12.37374 | 576.86 | 18.64 |
| 3 | 7.9518 | 11.11867 | 170.24 | 5.5 |
| 4 | 8.1769 | 10.81313 | 411.73 | 13.3 |
| 5 | 8.9403 | 9.89147 | 350.8 | 11.34 |
| 6 | 9.9236 | 8.91345 | 363.87 | 11.76 |
| 7 | 10.7005 | 8.26799 | 106.59 | 3.44 |
| 8 | 11.1775 | 7.91621 | 367.26 | 11.87 |
| 9 | 12.0671 | 7.33452 | 519.05 | 16.77 |
| 10 | 12.4835 | 7.09081 | 237.65 | 7.68 |
| 11 | 13.9186 | 6.36273 | 174.33 | 5.63 |
| 12 | 14.2867 | 6.19962 | 443.25 | 14.32 |
| 13 | 15.602 | 5.67982 | 509.86 | 16.48 |
| 14 | 15.8996 | 5.57415 | 237.92 | 7.69 |
| 15 | 16.3514 | 5.42115 | 599.32 | 19.37 |
| 16 | 17.4668 | 5.07739 | 295.01 | 9.53 |
| 17 | 17.9975 | 4.92886 | 317.75 | 10.27 |
| 18 | 18.4 | 4.82195 | 1880.67 | 60.77 |
| 19 | 19.9806 | 4.44392 | 229.96 | 7.43 |
| 20 | 21.5141 | 4.13051 | 180.3 | 5.83 |
| 21 | 22.035 | 4.03403 | 207.7 | 6.71 |
| 22 | 22.4546 | 3.95958 | 808.09 | 26.11 |
| 23 | 22.6194 | 3.9311 | 605.15 | 19.56 |
| 24 | 22.9688 | 3.8721 | 3094.59 | 100 |
| 25 | 23.2985 | 3.81803 | 336.49 | 10.87 |
| 26 | 23.7853 | 3.74098 | 412.94 | 13.34 |
| 27 | 24.2684 | 3.6676 | 1055.52 | 34.11 |
| 28 | 24.5233 | 3.63004 | 761.9 | 24.62 |
| 29 | 24.6781 | 3.60763 | 827.83 | 26.75 |
| 30 | 25.0584 | 3.55374 | 289.12 | 9.34 |
| 31 | 25.5793 | 3.48254 | 541.42 | 17.5 |
| 32 | 26.0905 | 3.41544 | 154.92 | 5.01 |
| 33 | 27.1775 | 3.28127 | 211.45 | 6.83 |
| 34 | 27.85 | 3.20354 | 331.47 | 10.71 |
| 35 | 28.3484 | 3.14834 | 310.69 | 10.04 |
| 36 | 29.6906 | 3.009 | 68.12 | 2.2 |
| 37 | 30.5903 | 2.92252 | 103.7 | 3.35 |
| 38 | 31.2858 | 2.85913 | 108.43 | 3.5 |
| 39 | 33.1836 | 2.69982 | 41.59 | 1.34 |
| 40 | 34.4205 | 2.60558 | 55.95 | 1.81 |

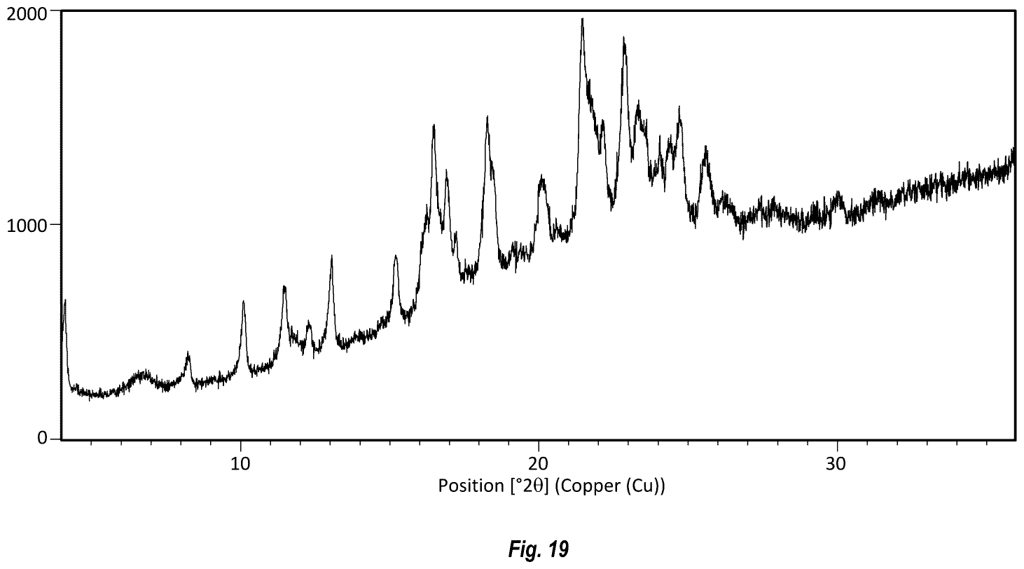

In one aspect, the present disclosure relates to Compound 1 hydrogen sulfate or a hydrate or solvate thereof, including crystalline Form 9 Compound 1 di(hydrogen sulfate) or a hydrate or solvate thereof, having an X-ray powder diffraction pattern comprising a peak at about 4.1° 2θ. In some embodiments, the Compound 1 hydrogen sulfate or hydrate or solvate thereof, including crystalline Form 9 Compound 1 di(hydrogen sulfate) or a hydrate or solvate thereof, has an X-ray powder diffraction pattern comprising one or more peaks chosen from peaks at about 8.3° 2θ, about 10.1° 2θ, about 11.5° 2θ, about 12.3° 2θ, about 13.1° 2θ, and about 16.5° 2θ. In some embodiments, the Compound 1 hydrogen sulfate or hydrate or solvate thereof, including crystalline Form 9 Compound 1 di(hydrogen sulfate) or a hydrate or solvate thereof, has an X-ray powder diffraction pattern comprising peaks at about 8.3° 2θ, about 10.1° 2θ, about 11.5° 2θ, about 12.3° 2θ, about 13.1° 2θ, and about 16.5° 2θ. In some embodiments, the Compound 1 hydrogen sulfate or hydrate or solvate thereof, including crystalline Form 9 Compound 1 di(hydrogen sulfate) or a hydrate or solvate thereof, has an X-ray powder diffraction pattern comprising peaks at about 4.1° 2θ, about 10.1° 2θ, and about 13.1° 2θ. In some embodiments, the Compound 1 hydrogen sulfate or hydrate or solvate thereof, including crystalline Form 9 Compound 1 di(hydrogen sulfate) or a hydrate or solvate thereof, has an X-ray powder diffraction pattern substantially the same as that of FIG. 19.

In one aspect, the present disclosure relates to crystalline Form 9 Compound 1 di(hydrogen sulfate) or a hydrate or solvate thereof. In some embodiments, the crystalline Form 9 Compound 1 di(hydrogen sulfate) or hydrate or solvate thereof has an X-ray powder diffraction pattern comprising one or more peaks chosen from the peak list in Table 14 below (all peaks that can be or have been chosen from Table 14 are rounded to the nearest 0.1° 2θ). In some embodiments, the crystalline Form 9 Compound 1 di(hydrogen sulfate) or hydrate or solvate thereof has an X-ray powder diffraction pattern comprising two or more peaks chosen from the peak list in Table 14 below (all peaks that can be or have been chosen from Table 14 are rounded to the nearest 0.1° 2θ). In some embodiments, the crystalline Form 9 Compound 1 di(hydrogen sulfate) or hydrate or solvate thereof has an X-ray powder diffraction pattern comprising three or more peaks chosen from the peak list in Table 14 below (all peaks that can be or have been chosen from Table 14 are rounded to the nearest 0.1° 2θ).

TABLE 14
Peak list for crystalline Form 9 Compound 1 di(hydrogen sulfate)

| No. | Pos. [° 2θ] | d-spacing [Å] | Height [cts] | Rel. Int. [%] |
|---|---|---|---|---|
| 1 | 4.1247 | 21.42274 | 452.41 | 38.22 |
| 2 | 6.7046 | 13.18389 | 63.9 | 5.4 |
| 3 | 8.2636 | 10.69993 | 127.19 | 10.75 |
| 4 | 10.0925 | 8.76461 | 341.28 | 28.83 |
| 5 | 11.4783 | 7.70937 | 350.15 | 29.58 |
| 6 | 12.302 | 7.195 | 141.71 | 11.97 |
| 7 | 13.0566 | 6.78082 | 421.3 | 35.59 |
| 8 | 15.207 | 5.82643 | 336.63 | 28.44 |
| 9 | 16.4796 | 5.37926 | 882.68 | 74.57 |
| 10 | 16.8973 | 5.24723 | 664.93 | 56.18 |
| 11 | 17.2418 | 5.14314 | 345.7 | 29.21 |
| 12 | 18.2481 | 4.86174 | 817.11 | 69.03 |
| 13 | 18.4888 | 4.79898 | 583.89 | 49.33 |
| 14 | 20.0347 | 4.43204 | 446.48 | 37.72 |
| 15 | 21.4556 | 4.14162 | 1183.65 | 100 |
| 16 | 22.1589 | 4.01175 | 643 | 54.32 |
| 17 | 22.8833 | 3.88637 | 982.81 | 83.03 |
| 18 | 23.318 | 3.81489 | 663.09 | 56.02 |
| 19 | 24.0503 | 3.70037 | 456.85 | 38.6 |
| 20 | 24.3305 | 3.65837 | 442.12 | 37.35 |
| 21 | 24.7396 | 3.5988 | 559.75 | 47.29 |
| 22 | 25.577 | 3.48284 | 353.39 | 29.86 |
| 23 | 30.0906 | 2.96991 | 75.83 | 6.41 |
| 24 | 31.2996 | 2.8579 | 39.74 | 3.36 |

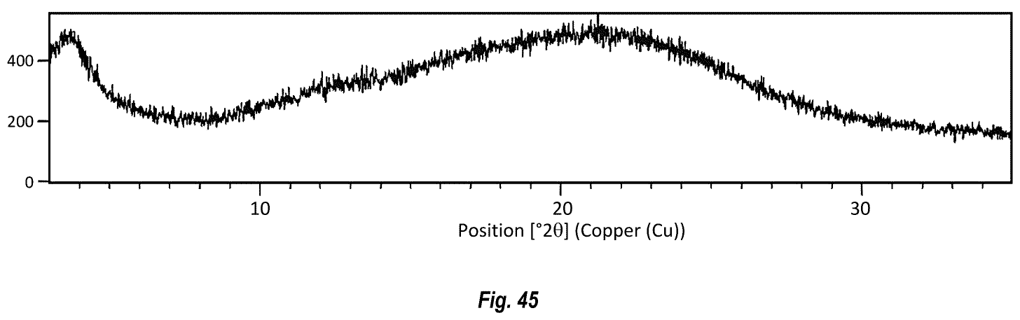

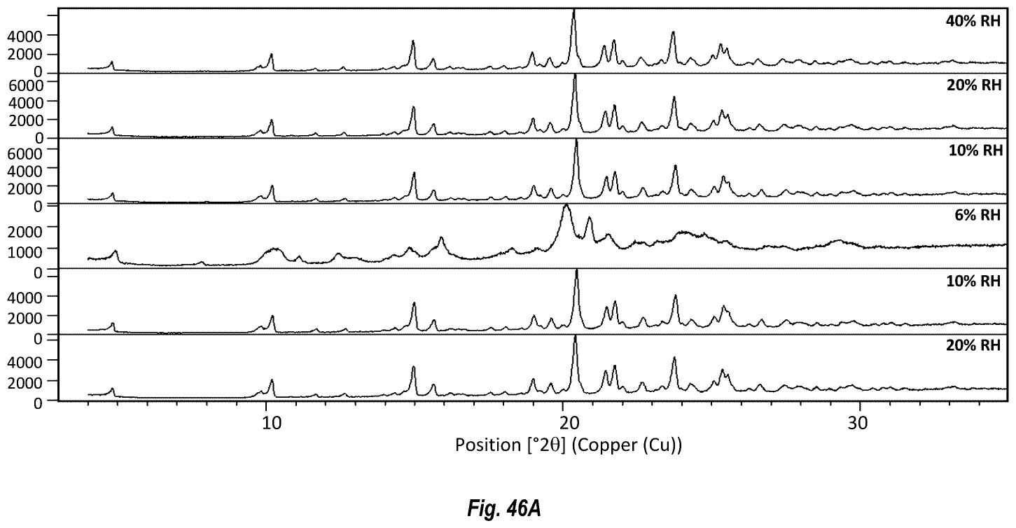

In one aspect, the present disclosure relates to amorphous Compound 1 di(hydrogen sulfate) and hydrates and solvates thereof. In some embodiments, the amorphous Compound 1 di(hydrogen sulfate) or hydrate or solvate thereof has an X-ray powder diffraction pattern substantially the same as that of FIG. 45.

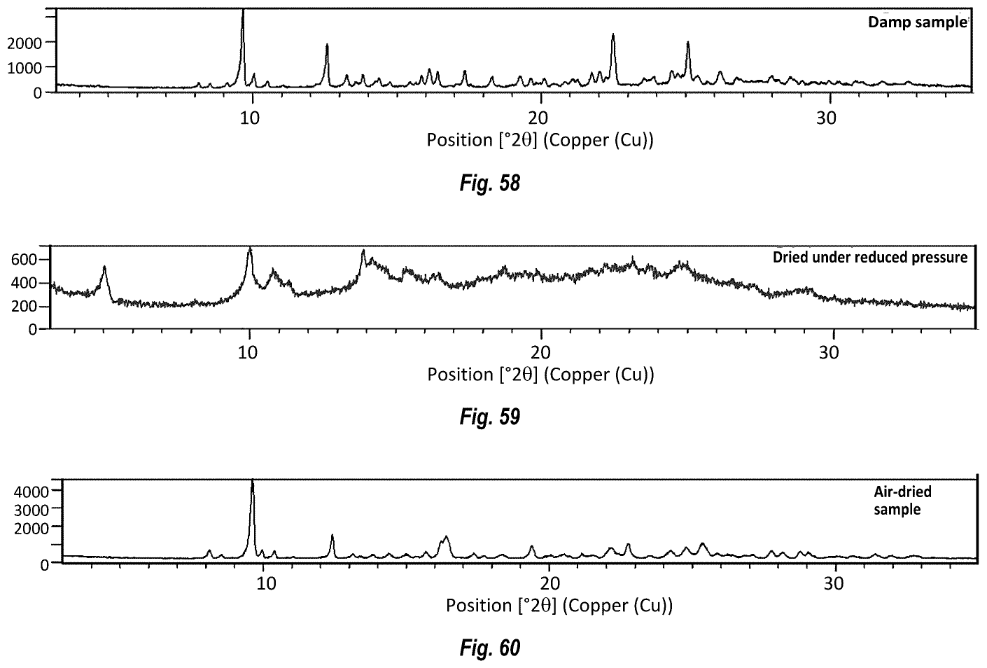

In one aspect, the present disclosure relates to Compound 1 hydrogen sulfate or a hydrate or solvate thereof, including crystalline Form 4 Compound 1 mono(hydrogen sulfate) or a hydrate or solvate thereof, having an X-ray powder diffraction pattern comprising a peak at about 8.0° 2θ. In some embodiments, the Compound 1 hydrogen sulfate or hydrate or solvate thereof, including crystalline Form 4 Compound 1 mono(hydrogen sulfate) or a hydrate or solvate thereof, has an X-ray powder diffraction pattern comprising a peak at about 4.5° 2θ. In some embodiments, the Compound 1 hydrogen sulfate or hydrate or solvate thereof, including crystalline Form 4 Compound 1 mono(hydrogen sulfate) or a hydrate or solvate thereof, has an X-ray powder diffraction pattern comprising one or more peaks chosen from peaks at about 4.5° 2θ, about 8.0° 2θ, about 9.0° 2θ, about 9.5° 2θ, about 10.4° 2θ, and about 12.4° 2θ. In some embodiments, the Compound 1 hydrogen sulfate or hydrate or solvate thereof, including crystalline Form 4 Compound 1 mono(hydrogen sulfate) or a hydrate or solvate thereof, has an X-ray powder diffraction pattern comprising peaks at about 8.0° 2θ, about 9.0° 2θ, about 10.4° 2θ, and about 12.4° 2θ. In some embodiments, the Compound 1 hydrogen sulfate or hydrate or solvate thereof, including crystalline Form 4 Compound 1 mono(hydrogen sulfate) or a hydrate or solvate thereof, has an X-ray powder diffraction pattern substantially the same as that of FIG. 58. In some embodiments, the Compound 1 hydrogen sulfate or hydrate or solvate thereof, including crystalline Form 4 Compound 1 mono(hydrogen sulfate) or a hydrate or solvate thereof, has an X-ray powder diffraction pattern substantially the same as that of FIG. 60.

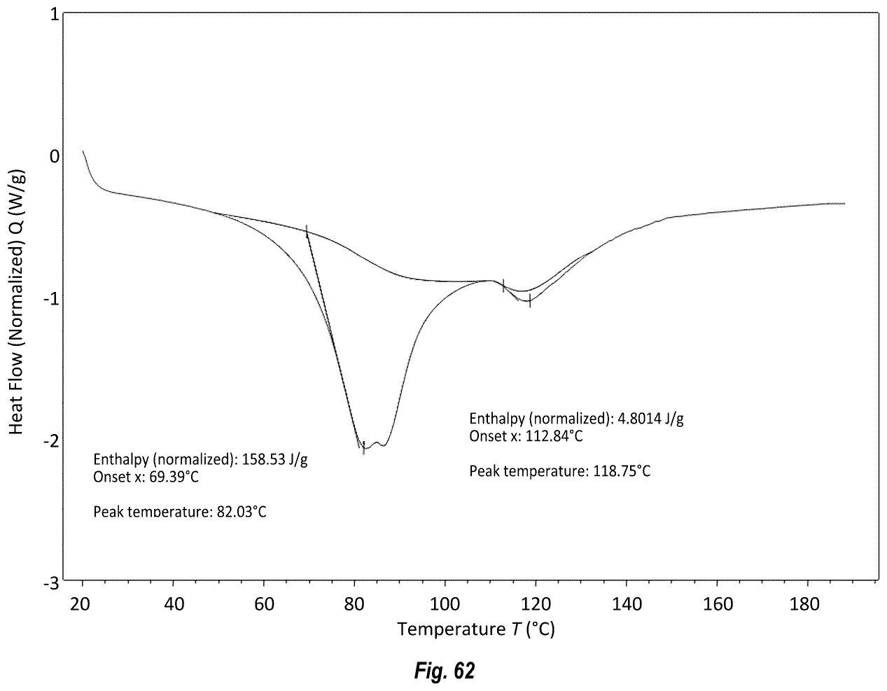

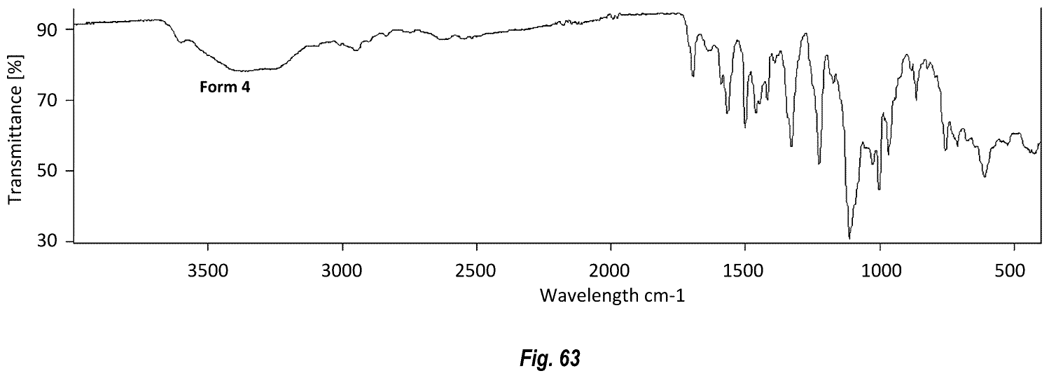

In some embodiments, the Compound 1 hydrogen sulfate or hydrate or solvate thereof, including crystalline Form 4 Compound 1 mono(hydrogen sulfate) or a hydrate or solvate thereof, has an FT-IR spectrum substantially the same as that of FIG. 63. In some embodiments, the Compound 1 hydrogen sulfate or hydrate or solvate thereof, including crystalline Form 4 Compound 1 mono(hydrogen sulfate) or a hydrate or solvate thereof, has a melting onset as measured by DSC in an open aluminum pan of about 148° C. In some embodiments, the Compound 1 hydrogen sulfate or hydrate or solvate thereof, including crystalline Form 4 Compound 1 mono(hydrogen sulfate) or a hydrate or solvate thereof, has a DSC thermogram substantially the same as that of FIG. 62.

In one aspect, the present disclosure relates crystalline Form 4 Compound 1 mono(hydrogen sulfate) and hydrates and solvates thereof. In some embodiments, the crystalline Form 4 Compound 1 mono(hydrogen sulfate) or a hydrate or solvate thereof is a hydrate. In some embodiments, the crystalline Form 4 Compound 1 mono(hydrogen sulfate) or a hydrate or solvate thereof is a septahydrate.

In some embodiments, the crystalline Form 4 Compound 1 mono(hydrogen sulfate) or hydrate or solvate thereof has an X-ray powder diffraction pattern comprising one or more peaks chosen from the peak list in Table 15 or 16 below (all peaks that can be or have been chosen from Table 15 or 16 are rounded to the nearest 0.1° 2θ). In some embodiments, the crystalline Form 4 Compound 1 mono(hydrogen sulfate) or hydrate or solvate thereof has an X-ray powder diffraction pattern comprising two or more peaks chosen from the peak list in Table 15 or 16 below (all peaks that can be or have been chosen from Table 15 or 16 are rounded to the nearest 0.1° 2θ). In some embodiments, the crystalline Form 4 Compound 1 mono(hydrogen sulfate) or hydrate or solvate thereof has an X-ray powder diffraction pattern comprising three or more peaks chosen from the peak list in Table 15 or 16 below (all peaks that can be or have been chosen from Table 15 or 16 are rounded to the nearest 0.1° 2θ).

TABLE 15
Peak list for crystalline Form 4
Compound 1 mono(hydrogen sulfate)

| No. | Pos. [° 2θ] | d-spacing [Å] | Height [cts] | Rel. Int. [%] |
|---|---|---|---|---|
| 1 | 7.982 | 11.07677 | 162.32 | 6.27 |
| 2 | 8.334 | 10.60959 | 129.16 | 4.99 |
| 3 | 8.9581 | 9.8718 | 148.26 | 5.73 |
| 4 | 9.5153 | 9.29501 | 2588.72 | 100 |
| 5 | 9.8851 | 8.94808 | 527.72 | 20.39 |
| 6 | 10.3687 | 8.5318 | 232.69 | 8.99 |
| 7 | 10.8196 | 8.17722 | 44.85 | 1.73 |
| 8 | 12.4352 | 7.11822 | 1387.69 | 53.61 |
| 9 | 13.1224 | 6.74697 | 433.22 | 16.74 |
| 10 | 13.4889 | 6.56447 | 193.32 | 7.47 |
| 11 | 13.6992 | 6.46414 | 418.72 | 16.17 |
| 12 | 14.2483 | 6.21626 | 331.17 | 12.79 |
| 13 | 14.6062 | 6.06472 | 211.59 | 8.17 |
| 14 | 15.3337 | 5.77858 | 184.99 | 7.15 |
| 15 | 15.7446 | 5.62869 | 405.4 | 15.66 |
| 16 | 15.997 | 5.54044 | 786.03 | 30.36 |
| 17 | 16.2756 | 5.44622 | 641.22 | 24.77 |
| 18 | 17.2507 | 5.14051 | 629.66 | 24.32 |
| 19 | 18.1638 | 4.88411 | 347.17 | 13.41 |
| 20 | 19.1852 | 4.62632 | 376.79 | 14.56 |
| 21 | 19.5381 | 4.54355 | 347.46 | 13.42 |
| 22 | 20.0169 | 4.43593 | 237.12 | 9.16 |
| 23 | 20.3602 | 4.36192 | 87.51 | 3.38 |
| 24 | 20.7383 | 4.28323 | 185.82 | 7.18 |
| 25 | 21.0252 | 4.22542 | 252.01 | 9.73 |
| 26 | 21.1991 | 4.19116 | 215.87 | 8.34 |
| 27 | 21.661 | 4.10281 | 429.04 | 16.57 |
| 28 | 21.9474 | 4.04993 | 519.88 | 20.08 |
| 29 | 22.4023 | 3.96871 | 1997.97 | 77.18 |
| 30 | 23.4819 | 3.78863 | 288.87 | 11.16 |
| 31 | 23.813 | 3.73669 | 346.51 | 13.39 |
| 32 | 24.4529 | 3.64033 | 523.4 | 20.22 |
| 33 | 24.6558 | 3.61084 | 411.54 | 15.9 |
| 34 | 25.0029 | 3.56149 | 1495.37 | 57.76 |
| 35 | 25.361 | 3.51202 | 394.94 | 15.26 |
| 36 | 25.6937 | 3.46729 | 178.96 | 6.91 |
| 37 | 26.1109 | 3.41283 | 500.36 | 19.33 |
| 38 | 26.7286 | 3.33534 | 348.11 | 13.45 |
| 39 | 27.9309 | 3.19445 | 325.81 | 12.59 |
| 40 | 28.5758 | 3.1238 | 326.24 | 12.6 |
| 41 | 28.9917 | 3.07993 | 193.7 | 7.48 |
| 42 | 29.4054 | 3.03754 | 142.12 | 5.49 |
| 43 | 29.9366 | 2.98483 | 175.14 | 6.77 |
| 44 | 30.2871 | 2.95109 | 162.82 | 6.29 |
| 45 | 30.8391 | 2.89951 | 214.42 | 8.28 |
| 46 | 31.1344 | 2.87268 | 145 | 5.6 |
| 47 | 31.8044 | 2.81368 | 181.78 | 7.02 |
| 48 | 32.7007 | 2.73858 | 150.36 | 5.81 |
| 49 | 33.8046 | 2.65163 | 63.8 | 2.46 |

TABLE 16
Peak list for crystalline Form 4
Compound 1 mono(hydrogen sulfate)

| No. | Pos. [° 2θ] | d-spacing [Å] | Height [cts] | Rel. Int. [%] |
|---|---|---|---|---|
| 1 | 4.4622 | 19.80321 | 103.82 | 2.58 |
| 2 | 8.2005 | 10.78203 | 260.52 | 6.48 |
| 3 | 8.6094 | 10.27092 | 184.75 | 4.6 |
| 4 | 8.9281 | 9.90498 | 247.96 | 6.17 |
| 5 | 9.6719 | 9.1448 | 4018.35 | 100 |
| 6 | 9.9912 | 8.85328 | 824.39 | 20.52 |
| 7 | 10.4307 | 8.4812 | 270.81 | 6.74 |
| 8 | 11.1027 | 7.96931 | 163.2 | 4.06 |
| 9 | 12.4495 | 7.11008 | 2898.9 | 72.14 |
| 10 | 13.1589 | 6.72831 | 658.69 | 16.39 |
| 11 | 13.4413 | 6.58759 | 330.42 | 8.22 |
| 12 | 13.8872 | 6.37706 | 714.09 | 17.77 |
| 13 | 14.4675 | 6.12257 | 693.95 | 17.27 |
| 14 | 15.0926 | 5.87033 | 275.19 | 6.85 |
| 15 | 15.4249 | 5.74463 | 358.23 | 8.91 |
| 16 | 15.7037 | 5.64325 | 524.76 | 13.06 |
| 17 | 16.2311 | 5.46104 | 407.49 | 10.14 |
| 18 | 16.4525 | 5.38805 | 699.66 | 17.41 |
| 19 | 16.9502 | 5.23095 | 282.82 | 7.04 |
| 20 | 17.4598 | 5.07941 | 1271.35 | 31.64 |
| 21 | 18.3932 | 4.82371 | 503.77 | 12.54 |
| 22 | 19.4525 | 4.56336 | 1011.24 | 25.17 |
| 23 | 20.0909 | 4.41977 | 455.35 | 11.33 |
| 24 | 20.4646 | 4.33989 | 281.47 | 7 |
| 25 | 21.3688 | 4.15825 | 263.09 | 6.55 |
| 26 | 22.1893 | 4.00631 | 1173.47 | 29.2 |
| 27 | 22.5302 | 3.94647 | 837.29 | 20.84 |
| 28 | 22.8609 | 3.89012 | 2788.18 | 69.39 |
| 29 | 23.578 | 3.77341 | 476.78 | 11.87 |
| 30 | 24.2912 | 3.66421 | 341.51 | 8.5 |
| 31 | 24.8976 | 3.57632 | 843.39 | 20.99 |
| 32 | 25.4697 | 3.49727 | 2402.88 | 59.8 |
| 33 | 25.9587 | 3.43249 | 272.64 | 6.78 |
| 34 | 26.5803 | 3.35362 | 698.82 | 17.39 |
| 35 | 26.8971 | 3.31483 | 556.3 | 13.84 |
| 36 | 27.7723 | 3.21232 | 771.78 | 19.21 |
| 37 | 28.2003 | 3.16454 | 284.24 | 7.07 |
| 38 | 28.813 | 3.09863 | 322.7 | 8.03 |
| 39 | 29.1137 | 3.0673 | 398.86 | 9.93 |
| 40 | 29.8206 | 2.99618 | 246.28 | 6.13 |
| 41 | 31.5457 | 2.83616 | 153.98 | 3.83 |
| 42 | 31.9968 | 2.79719 | 211.51 | 5.26 |
| 43 | 32.7977 | 2.7307 | 158.31 | 3.94 |
| 44 | 33.5537 | 2.67088 | 81.1 | 2.02 |

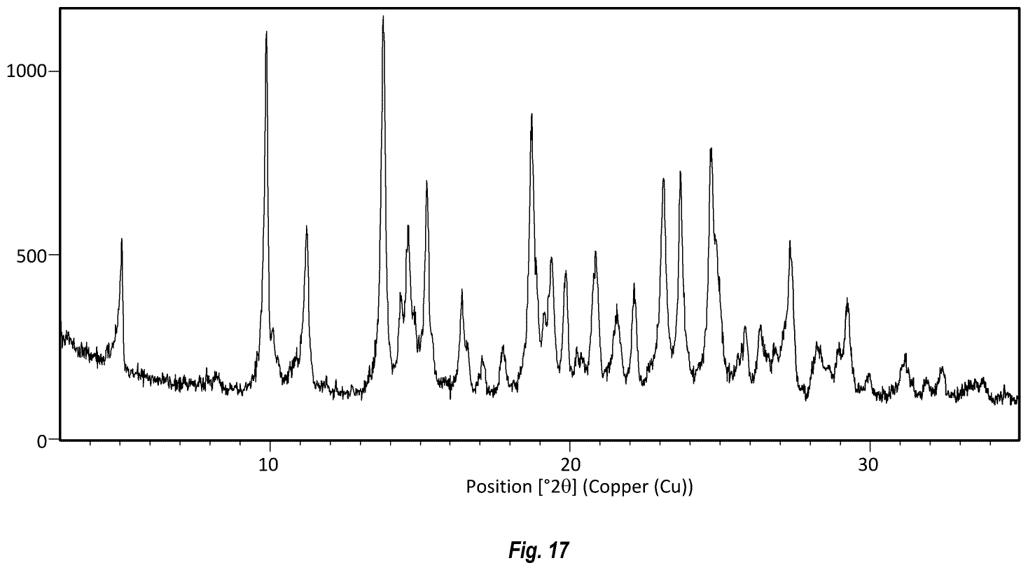

In one aspect, the present disclosure relates to Compound 1 hydrogen sulfate or a hydrate or solvate thereof, including crystalline Form 8 Compound 1 mono(hydrogen sulfate) or a hydrate or solvate thereof, having an X-ray powder diffraction pattern comprising a peak at about 5.1° 2θ. In some embodiments, the Compound 1 hydrogen sulfate or hydrate or solvate thereof, including crystalline Form 8 Compound 1 mono(hydrogen sulfate) or a hydrate or solvate thereof, has an X-ray powder diffraction pattern comprising a peak at about 5.1° 2θ and one or more peaks chosen from peaks at about 9.9° 2θ, about 11.2° 2θ, and about 13.8° 2θ. In some embodiments, the Compound 1 hydrogen sulfate or hydrate or solvate thereof, including crystalline Form 8 Compound 1 mono(hydrogen sulfate) or a hydrate or solvate thereof, has an X-ray powder diffraction pattern comprising peaks at about 5.1° 2θ, about 9.9° 2θ, about 11.2° 2θ, and about 13.8° 2θ. In some embodiments, the Compound 1 hydrogen sulfate or hydrate or solvate thereof, including crystalline Form 8 Compound 1 mono(hydrogen sulfate) or a hydrate or solvate thereof, has an X-ray powder diffraction pattern substantially the same as that of FIG. 17.

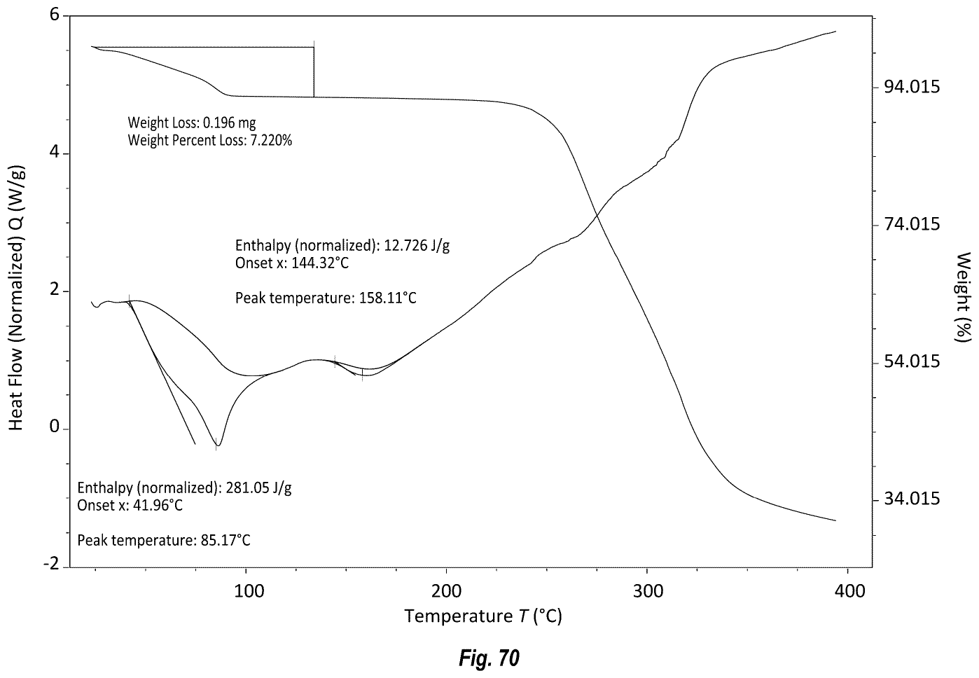

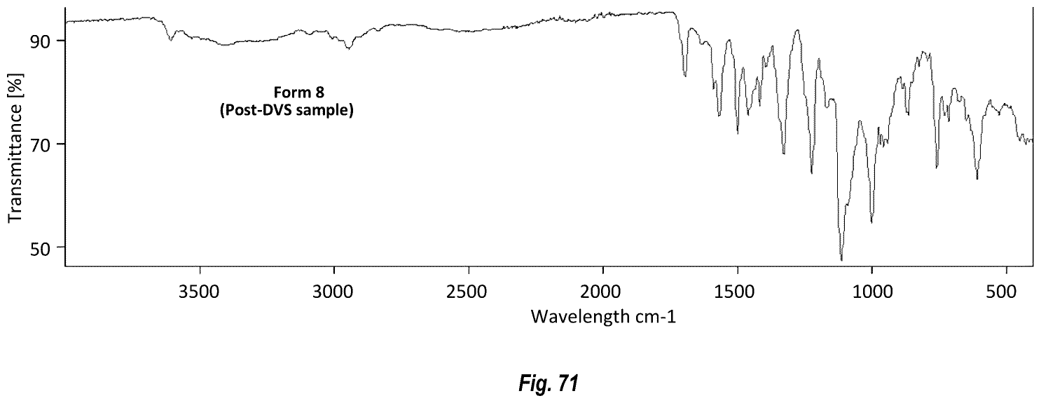

In some embodiments, the Compound 1 hydrogen sulfate or hydrate or solvate thereof, including crystalline Form 8 Compound 1 mono(hydrogen sulfate or a hydrate or solvate thereof), has an FT-IR spectrum substantially the same as that of FIG. 71. In some embodiments, the Compound 1 hydrogen sulfate or hydrate or solvate thereof, including crystalline Form 8 Compound 1 mono(hydrogen sulfate) or a hydrate or solvate thereof, has a melting onset as measured by DSC in an open aluminum pan of about 144° C. In some embodiments, the Compound 1 hydrogen sulfate or hydrate or solvate thereof, including crystalline Form 8 Compound 1 mono(hydrogen sulfate) or a hydrate or solvate thereof, has a DSC thermogram substantially the same as that of FIG. 70.

In one aspect, the present disclosure relates to crystalline Form 8 Compound 1 mono(hydrogen sulfate) and hydrates and solvates thereof. In some embodiments, the crystalline Form 8 Compound 1 mono(hydrogen sulfate) or a hydrate or solvate thereof is a hydrate. In some embodiments, the crystalline Form 8 Compound 1 mono(hydrogen sulfate) or a hydrate or solvate thereof is a trihydrate.

In some embodiments, the crystalline Form 8 Compound 1 mono(hydrogen sulfate) or hydrate or solvate thereof has an X-ray powder diffraction pattern comprising one or more peaks chosen from the peak list in Table 17 below (all peaks that can be or have been chosen from Table 17 are rounded to the nearest 0.1° 2θ). In some embodiments, the crystalline Form 8 Compound 1 mono(hydrogen sulfate) or hydrate or solvate thereof has an X-ray powder diffraction pattern comprising two or more peaks chosen from the peak list in Table 17 below (all peaks that can be or have been chosen from Table 17 are rounded to the nearest 0.1° 2θ). In some embodiments, the crystalline Form 8 Compound 1 mono(hydrogen sulfate) or hydrate or solvate thereof has an X-ray powder diffraction pattern comprising three or more peaks chosen from the peak list in Table 17 below (all peaks that can be or have been chosen from Table 17 are rounded to the nearest 0.1° 2θ).

TABLE 17
Peak list for crystalline Form 8
Compound 1 mono(hydrogen sulfate)

| No. | Pos. [° 2θ] | d-spacing [Å] | Height [cts] | Rel. Int. [%] |
|---|---|---|---|---|
| 1 | 5.0644 | 17.44975 | 352.23 | 35.47 |
| 2 | 8.1899 | 10.79605 | 31 | 3.12 |
| 3 | 9.8838 | 8.94927 | 992.91 | 100 |
| 4 | 11.2376 | 7.87396 | 431.52 | 43.46 |
| 5 | 13.8084 | 6.41325 | 959.35 | 96.62 |
| 6 | 14.3466 | 6.17387 | 258.6 | 26.04 |
| 7 | 14.6094 | 6.06341 | 467.97 | 47.13 |
| 8 | 15.2198 | 5.82155 | 595.1 | 59.94 |
| 9 | 16.3888 | 5.40886 | 265.15 | 26.7 |
| 10 | 17.0886 | 5.18891 | 89.77 | 9.04 |
| 11 | 17.7546 | 4.99575 | 130.41 | 13.13 |
| 12 | 18.7095 | 4.74287 | 766.35 | 77.18 |
| 13 | 19.4122 | 4.57274 | 353.01 | 35.55 |
| 14 | 19.8644 | 4.46965 | 336.46 | 33.89 |
| 15 | 20.8861 | 4.25326 | 352.21 | 35.47 |
| 16 | 21.5656 | 4.12076 | 229.75 | 23.14 |
| 17 | 22.1387 | 4.01537 | 290.18 | 29.23 |
| 18 | 23.1187 | 3.84731 | 600.72 | 60.5 |
| 19 | 23.6884 | 3.75606 | 618.9 | 62.33 |
| 20 | 24.6647 | 3.60956 | 678.09 | 68.29 |
| 21 | 25.8431 | 3.44758 | 194.35 | 19.57 |
| 22 | 26.324 | 3.38568 | 189.03 | 19.04 |
| 23 | 27.3482 | 3.26117 | 408.05 | 41.1 |
| 24 | 28.2685 | 3.15706 | 133.84 | 13.48 |
| 25 | 29.2464 | 3.05369 | 267.58 | 26.95 |
| 26 | 29.9484 | 2.98368 | 61.19 | 6.16 |
| 27 | 31.134 | 2.87271 | 104.57 | 10.53 |
| 28 | 31.8709 | 2.80796 | 48.73 | 4.91 |
| 29 | 32.4491 | 2.75923 | 71.06 | 7.16 |
| 30 | 33.7271 | 2.65755 | 42.05 | 4.24 |

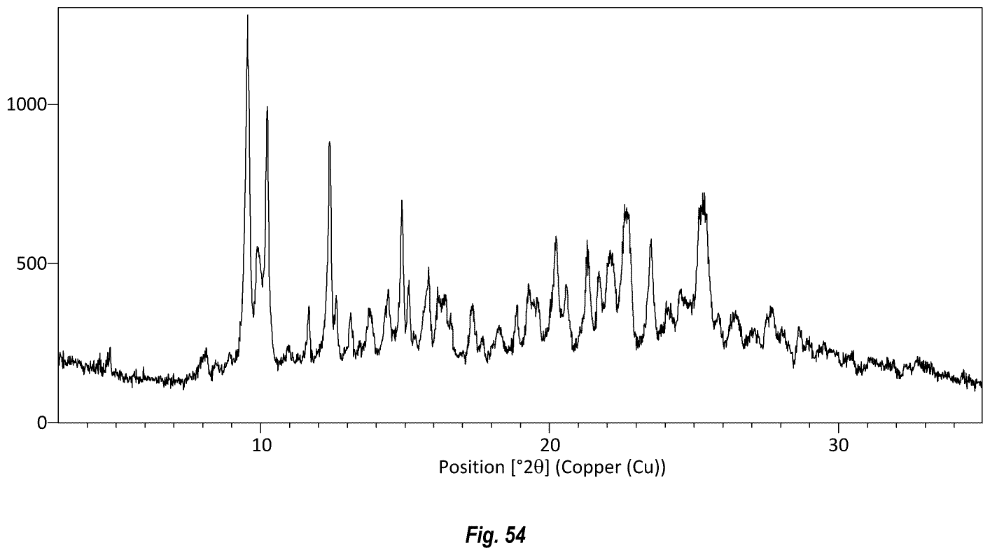

In one aspect, the present disclosure relates to Compound 1 hydrogen sulfate or hydrate or solvate thereof, including crystalline Form 10 Compound 1 mono(hydrogen sulfate) or a hydrate or solvate thereof, having an X-ray powder diffraction pattern comprising a peak at about 11.7° 2θ. In some embodiments, the Compound 1 hydrogen sulfate or hydrate or solvate thereof, including crystalline Form 10 Compound 1 mono(hydrogen sulfate) or a hydrate or solvate thereof, has an X-ray powder diffraction pattern comprising one or more peaks at about 9.6° 2θ, about 10.2° 2θ, about 11.7° 2θ, about 12.4° 2θ, and about 13.1° 2θ. In some embodiments, the Compound 1 hydrogen sulfate or hydrate or solvate thereof, including crystalline Form 10 Compound 1 mono(hydrogen sulfate) or a hydrate or solvate thereof, has an X-ray powder diffraction pattern comprising peaks at about 9.6° 2θ, about 10.2° 2θ, about 11.7° 2θ, about 12.4° 2θ, and about 13.1° 2θ. In some embodiments, the Compound 1 hydrogen sulfate or hydrate or solvate thereof, including crystalline Form 10 Compound 1 mono(hydrogen sulfate) or a hydrate or solvate thereof, has an X-ray powder diffraction pattern substantially the same as that of FIG. 54.

In one aspect, the present disclosure relates crystalline Form 10 Compound 1 mono(hydrogen sulfate) or hydrate or solvate thereof.

In some embodiments, the crystalline Form 10 Compound 1 mono(hydrogen sulfate) or hydrate or solvate thereof has an X-ray powder diffraction pattern comprising one or more peaks chosen from the peak list in Table 18 below (all peaks that can be or have been chosen from Table 18 are rounded to the nearest 0.1° 2θ). In some embodiments, the crystalline Form 10 Compound 1 mono(hydrogen sulfate) or hydrate or solvate thereof has an X-ray powder diffraction pattern comprising two or more peaks chosen from the peak list in Table 18 below (all peaks that can be or have been chosen from Table 18 are rounded to the nearest 0.1° 2θ). In some embodiments, the crystalline Form 10 Compound 1 mono(hydrogen sulfate) or hydrate or solvate thereof has an X-ray powder diffraction pattern comprising three or more peaks chosen from the peak list in Table 18 below (all peaks that can be or have been chosen from Table 18 are rounded to the nearest 0.1° 2θ).

TABLE 18
Peak list for crystalline Form 10
Compound 1 mono(hydrogen sulfate)

| No. | Pos. [° 2θ] | d-spacing [Å] | Height [cts] | Rel. Int. [%] |
|---|---|---|---|---|
| 1 | 4.7899 | 18.44898 | 77.53 | 6.59 |
| 2 | 8.0946 | 10.92284 | 87.13 | 7.41 |
| 3 | 9.5578 | 9.25373 | 1176.03 | 100 |
| 4 | 9.8784 | 8.95416 | 427.98 | 36.39 |
| 5 | 10.2354 | 8.64256 | 881.28 | 74.94 |
| 6 | 11.6766 | 7.57892 | 239.78 | 20.39 |
| 7 | 12.3936 | 7.142 | 767.88 | 65.29 |
| 8 | 12.624 | 7.01216 | 264.01 | 22.45 |
| 9 | 13.1158 | 6.75034 | 208.63 | 17.74 |
| 10 | 13.7709 | 6.43065 | 222.02 | 18.88 |
| 11 | 14.4268 | 6.13973 | 285.71 | 24.29 |
| 12 | 14.8992 | 5.9461 | 580.75 | 49.38 |
| 13 | 15.1236 | 5.8584 | 280.43 | 23.85 |
| 14 | 15.8174 | 5.60293 | 354.5 | 30.14 |
| 15 | 16.1552 | 5.48654 | 261.53 | 22.24 |
| 16 | 17.3094 | 5.1232 | 230.17 | 19.57 |
| 17 | 18.2136 | 4.87087 | 159.06 | 13.53 |
| 18 | 18.8663 | 4.70381 | 236.92 | 20.15 |
| 19 | 19.2604 | 4.60842 | 286.8 | 24.39 |
| 20 | 20.2448 | 4.38652 | 453.04 | 38.52 |
| 21 | 20.5826 | 4.31527 | 308.49 | 26.23 |
| 22 | 21.3096 | 4.16968 | 398.5 | 33.89 |
| 23 | 21.7009 | 4.09536 | 341.15 | 29.01 |
| 24 | 22.0101 | 4.03853 | 373.92 | 31.8 |
| 25 | 22.6028 | 3.93395 | 540.77 | 45.98 |
| 26 | 22.7529 | 3.90834 | 515.35 | 43.82 |
| 27 | 23.4936 | 3.78677 | 433 | 36.82 |
| 28 | 24.1032 | 3.69236 | 222.98 | 18.96 |
| 29 | 24.5285 | 3.6293 | 285.12 | 24.24 |
| 30 | 25.1759 | 3.53742 | 529.05 | 44.99 |
| 31 | 25.4149 | 3.50469 | 526.19 | 44.74 |
| 32 | 25.8529 | 3.4463 | 206.17 | 17.53 |
| 33 | 26.4849 | 3.36547 | 202.49 | 17.22 |
| 34 | 27.7092 | 3.21949 | 225.05 | 19.14 |
| 35 | 28.6217 | 3.11889 | 167.75 | 14.26 |
| 36 | 31.1652 | 2.86992 | 63.81 | 5.43 |
| 37 | 32.8129 | 2.72947 | 50.49 | 4.29 |

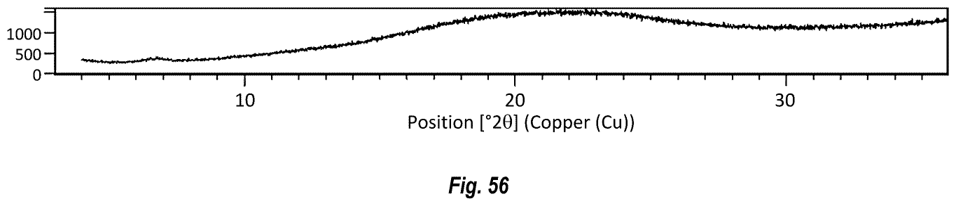

In one aspect, the present disclosure relates to amorphous Compound 1 mono(hydrogen sulfate) and hydrates and solvates thereof. In some embodiments, the Compound 1 mono(hydrogen sulfate) or hydrate or solvate thereof has an X-ray powder diffraction pattern substantially the same as that of FIG. 56.

In one aspect, the present disclosure relates to substantially crystalline Compound 1 hydrogen sulfate and hydrates and solvates thereof. In some embodiments, the substantially crystalline Compound 1 hydrogen sulfate or hydrate or solvate thereof is more than about 50% crystalline. In some embodiments, the substantially crystalline Compound 1 hydrogen sulfate or hydrate or solvate thereof is more than about 60% crystalline. In some embodiments, the substantially crystalline Compound 1 hydrogen sulfate or hydrate or solvate thereof is more than about 75% crystalline. In some embodiments, the substantially crystalline Compound 1 hydrogen sulfate or hydrate or solvate thereof is more than about 90% crystalline. In some embodiments, the substantially crystalline Compound 1 hydrogen sulfate or hydrate or solvate thereof is more than about 95% crystalline. In some embodiments, the substantially crystalline Compound 1 hydrogen sulfate or hydrate or solvate thereof is more than about 99% crystalline.

In one aspect, the present disclosure relates to substantially crystalline Compound 1 di(hydrogen sulfate) and hydrates and solvates thereof. In some embodiments, the substantially crystalline Compound 1 di(hydrogen sulfate) or hydrate or solvate thereof is more than about 50% crystalline. In some embodiments, the substantially crystalline Compound 1 di(hydrogen sulfate) or hydrate or solvate thereof is more than about 60% crystalline. In some embodiments, the substantially crystalline Compound 1 di(hydrogen sulfate) or hydrate or solvate thereof is more than about 75% crystalline. In some embodiments, the substantially crystalline Compound 1 di(hydrogen sulfate) or hydrate or solvate thereof is more than about 90% crystalline. In some embodiments, the substantially crystalline Compound 1 di(hydrogen sulfate) or hydrate or solvate thereof is more than about 95% crystalline. In some embodiments, the substantially crystalline Compound 1 di(hydrogen sulfate) or hydrate or solvate thereof is more than about 99% crystalline.

In one aspect, the present disclosure relates to substantially crystalline Form 2 Compound 1 di(hydrogen sulfate) trihydrate. In some embodiments, the substantially crystalline Form 2 Compound 1 di(hydrogen sulfate) trihydrate is more than about 50% crystalline. In some embodiments, the substantially crystalline Form 2 Compound 1 di(hydrogen sulfate) trihydrate is more than about 60% crystalline. In some embodiments, the substantially crystalline Form 2 Compound 1 di(hydrogen sulfate) trihydrate is more than about 75% crystalline. In some embodiments, the substantially crystalline Form 2 Compound 1 di(hydrogen sulfate) trihydrate is more than about 90% crystalline. In some embodiments, the substantially crystalline Form 2 Compound 1 di(hydrogen sulfate) trihydrate is more than about 95% crystalline. In some embodiments, the substantially crystalline Form 2 Compound 1 di(hydrogen sulfate) trihydrate is more than about 99% crystalline.

In one aspect, the present disclosure relates to substantially crystalline Compound 1 mono(hydrogen sulfate) and hydrates and solvates thereof. In some embodiments, the substantially crystalline Compound 1 mono(hydrogen sulfate) or hydrate or solvate thereof is more than about 50% crystalline. In some embodiments, the substantially crystalline Compound 1 mono(hydrogen sulfate) or hydrate or solvate thereof is more than about 60% crystalline. In some embodiments, the substantially crystalline Compound 1 mono(hydrogen sulfate) or hydrate or solvate thereof is more than about 75% crystalline. In some embodiments, the substantially crystalline Compound 1 mono(hydrogen sulfate) or hydrate or solvate thereof is more than about 90% crystalline. In some embodiments, the substantially crystalline Compound 1 mono(hydrogen sulfate) or hydrate or solvate thereof is more than about 95% crystalline. In some embodiments, the substantially crystalline Compound 1 mono(hydrogen sulfate) or hydrate or solvate thereof is more than about 99% crystalline.

In one aspect, the present disclosure relates to Compound 1 hydrogen chloride and hydrates and solvates thereof. Compound 1 hydrogen chloride is formed by, for example, the combination of Compound 1 (free base) and hydrochloric acid and can be represented as shown below where n is a number greater than zero.

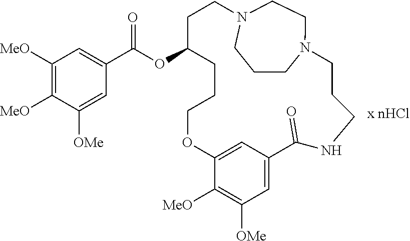 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 mono(hydrogen chloride) and hydrates and solvates thereof. Compound 1 mono(hydrogen chloride) is formed by, for example, the combination of Compound 1 (free base) and one molecular equivalent of hydrochloric acid and can be represented as shown below.

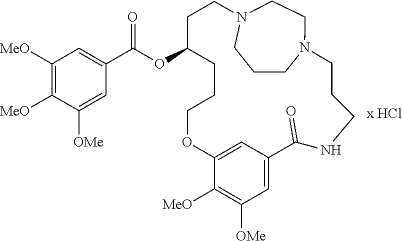 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 di(hydrogen chloride) and hydrates and solvates thereof. Compound 1 di(hydrogen chloride) is formed by, for example, the combination of Compound 1 (free base) and two molecular equivalents of hydrochloric acid and can be represented as shown below.

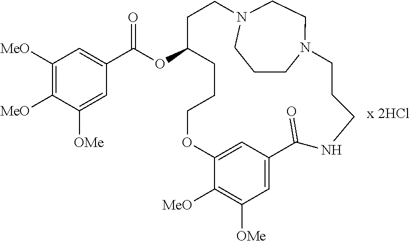 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to amorphous Compound 1 hydrogen chloride and hydrates and solvates thereof. In one aspect, the present disclosure relates to amorphous Compound 1 mono(hydrogen chloride) and hydrates and solvates thereof. In one aspect, the present disclosure relates to amorphous Compound 1 di(hydrogen chloride) and hydrates and solvates thereof.

In some embodiments, the present disclosure relates to crystalline Compound 1 hydrogen chloride and hydrates and solvates thereof. In some embodiments, the present disclosure relates to crystalline Compound 1 mono(hydrogen chloride) and hydrates and solvates thereof. In some embodiments, the present disclosure relates to crystalline Compound 1 di(hydrogen chloride) and hydrates and solvates thereof.

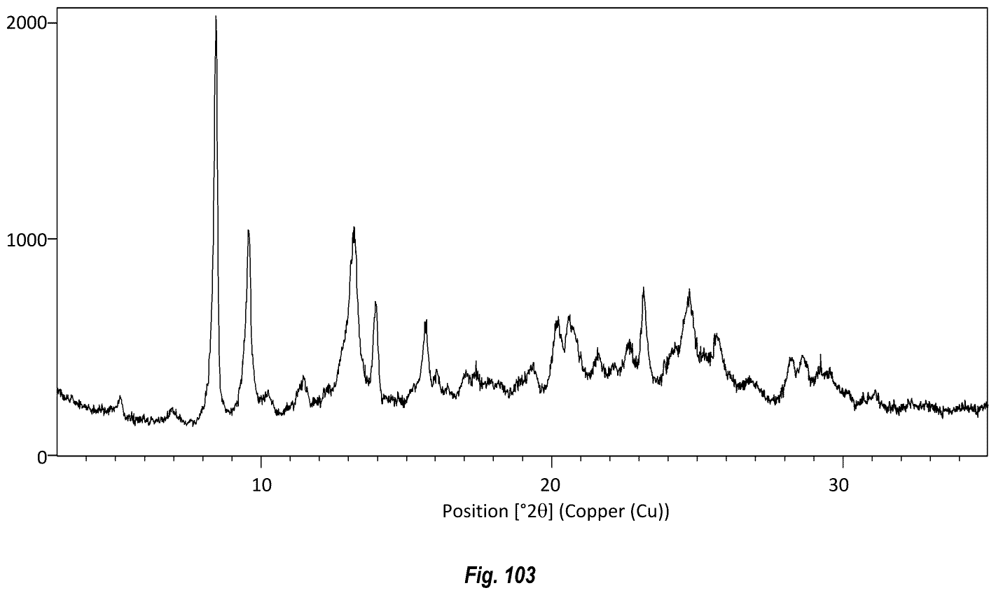

In some embodiments, the present disclosure relates to crystalline Compound 1 hydrogen chloride and hydrates and solvates thereof and crystalline Compound 1 di(hydrogen chloride) and hydrates and solvates thereof, including crystalline Form 1 Compound 1 hydrogen chloride or a hydrate or solvate thereof, having an x-ray powder diffraction pattern comprising a peak at about 8.5° 2θ. In some embodiments, the crystalline Compound 1 hydrogen chloride or hydrate or solvate thereof or the crystalline Compound 1 di(hydrogen chloride) or hydrate or solvate thereof, including crystalline Form 1 Compound 1 hydrogen chloride or a hydrate or solvate thereof, has an x-ray powder diffraction pattern comprising peaks at about 8.5° 2θ, about 9.6° 2θ, about 13.2° 2θ, about 14.0° 2θ, and about 15.7° 2θ. In some embodiments, the crystalline Compound 1 hydrogen chloride or hydrate or solvate thereof or the crystalline Compound 1 di(hydrogen chloride) or hydrate or solvate thereof, including crystalline Form 1 Compound 1 hydrogen chloride or a hydrate or solvate thereof, has an x-ray powder diffraction pattern comprising one or more peaks chosen from about 8.5° 2θ, about 9.6° 2θ, about 13.2° 2θ, about 14.0° 2θ, and about 15.7° 2θ. In some embodiments, the crystalline Compound 1 hydrogen chloride or hydrate or solvate thereof or the crystalline Compound 1 di(hydrogen chloride) or hydrate or solvate thereof, including crystalline Form 1 Compound 1 hydrogen chloride or a hydrate or solvate thereof, has an x-ray powder diffraction pattern substantially the same as that of FIG. 103.

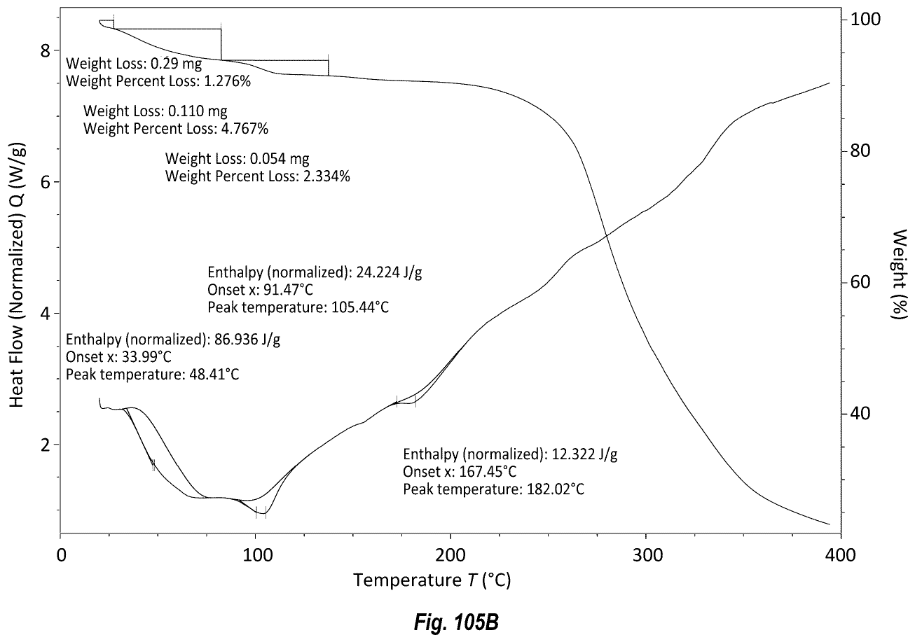

In some embodiments, the crystalline Compound 1 hydrogen chloride or hydrate or solvate thereof or the crystalline Compound 1 di(hydrogen chloride) or hydrate or solvate thereof, including crystalline Form 1 Compound 1 hydrogen chloride or a hydrate or solvate thereof, has a DSC endotherm with an onset of about 163° C. In some embodiments, the endotherm is a melting endotherm. In some embodiments, the crystalline Compound 1 hydrogen chloride or hydrate or solvate thereof or the crystalline Compound 1 di(hydrogen chloride) or hydrate or solvate thereof, including crystalline Form 1 Compound 1 hydrogen chloride or a hydrate or solvate thereof, has a DSC thermogram substantially the same as that of FIG. 105A or FIG. 105B.

In some embodiments, the present disclosure relates to crystalline Form 1 Compound 1 hydrogen chloride and hydrates and solvates thereof. In some embodiments, the crystalline Compound 1 hydrogen chloride or hydrate or solvate thereof or Compound 1 di(hydrogen chloride) or hydrate or solvate thereof is a hydrate.

In some embodiments, the crystalline Form 1 Compound 1 hydrogen chloride or hydrate or solvate thereof has an x-ray powder diffraction pattern comprising one or more peaks chosen from the peaks listed in Table 19 below (all peaks that can be or have been chosen from Table 19 are rounded to the nearest 0.1° 2θ). In some embodiments, the crystalline Form 1 Compound 1 hydrogen chloride or hydrate or solvate thereof has an x-ray powder diffraction pattern comprising two or more peaks chosen from the peaks listed in Table 19 below (all peaks that can be or have been chosen from Table 19 are rounded to the nearest 0.1° 2θ). In some embodiments, the crystalline Form 1 Compound 1 hydrogen chloride or hydrate or solvate thereof has an x-ray powder diffraction pattern comprising three or more peaks chosen from the peaks listed in Table 19 below (all peaks that can be or have been chosen from Table 19 are rounded to the nearest 0.1° 2θ).

TABLE 19
Peak list of crystalline Form 1 Compound 1 hydrogen chloride

| No. | Pos. [°2θ] | d-spacing [Å] | Height [cts] | Rel. Int. [%] |
|---|---|---|---|---|
| 1 | 5.1676 | 17.10145 | 83.71 | 4.36 |
| 2 | 6.9829 | 12.6592 | 50.31 | 2.62 |
| 3 | 8.4599 | 10.45201 | 1921.86 | 100 |
| 4 | 9.5717 | 9.24036 | 906.04 | 47.14 |
| 5 | 10.2439 | 8.63545 | 129.59 | 6.74 |
| 6 | 11.4929 | 7.69963 | 193.34 | 10.06 |
| 7 | 13.2234 | 6.69565 | 865.38 | 45.03 |
| 8 | 13.9661 | 6.34119 | 541.56 | 28.18 |
| 9 | 15.6646 | 5.65724 | 443.03 | 23.05 |
| 10 | 17.034 | 5.20542 | 205.63 | 10.7 |
| 11 | 19.3817 | 4.57986 | 231.52 | 12.05 |
| 12 | 20.1681 | 4.40302 | 436.71 | 22.72 |
| 13 | 20.5899 | 4.31376 | 467.21 | 24.31 |
| 14 | 21.5778 | 4.11845 | 289.07 | 15.04 |
| 15 | 22.7322 | 3.91185 | 314.38 | 16.36 |
| 16 | 23.1587 | 3.84076 | 581.24 | 30.24 |
| 17 | 24.7405 | 3.59867 | 545.41 | 28.38 |
| 18 | 25.6796 | 3.46916 | 367.33 | 19.11 |
| 19 | 26.8662 | 3.31857 | 153.22 | 7.97 |
| 20 | 28.1705 | 3.16782 | 244.06 | 12.7 |
| 21 | 28.6497 | 3.11591 | 263.06 | 13.69 |
| 22 | 29.3546 | 3.04268 | 185.85 | 9.67 |
| 23 | 31.0968 | 2.87607 | 80.92 | 4.21 |
| 24 | 32.6225 | 2.74496 | 24.32 | 1.27 |

In one aspect, the present disclosure relates to Compound 1 hydrogen phosphate and hydrates and solvates thereof. Compound 1 hydrogen phosphate is formed by, for example, the combination of Compound 1 (free base) and phosphoric acid and can be represented as shown below where n is a number greater than zero.

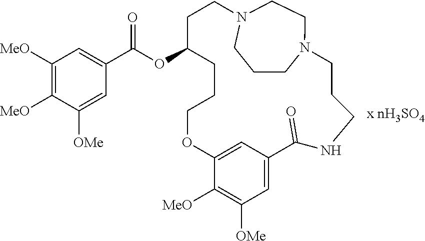 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 mono(hydrogen phosphate) and hydrates and solvates thereof. Compound 1 mono(hydrogen phosphate) is formed by, for example, the combination of Compound 1 (free base) and one molecular equivalent of phosphoric acid and can be represented as shown below.

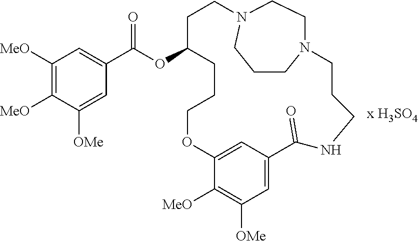 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 di(hydrogen phosphate) and hydrates and solvates thereof. Compound 1 mono(hydrogen phosphate) is formed by, for example, the combination of Compound 1 (free base) and two molecular equivalents of phosphoric acid and can be represented as shown below.

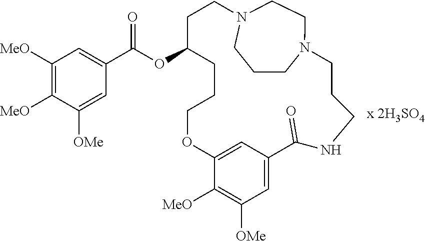 [SMILES: COc1cc(C(=O)O[C@@H]2CCCOc3cc(cc(OC)c3OC)C(=O)NCCCN3CCCN(CC2)CC3)cc(OC)c1OC]

In many embodiments, the Compound 1 di(hydrogen phosphate) or hydrate or solvate thereof is a di(dihydrogen phosphate) or hydrate or solvate thereof.

In one aspect, the present disclosure relates to amorphous Compound 1 hydrogen phosphate and hydrates and solvates thereof. In one aspect, the present disclosure relates to amorphous Compound 1 mono(hydrogen phosphate) and hydrates and solvates thereof. In one aspect, the present disclosure relates to amorphous Compound 1 di(hydrogen phosphate) and hydrates and solvates thereof.

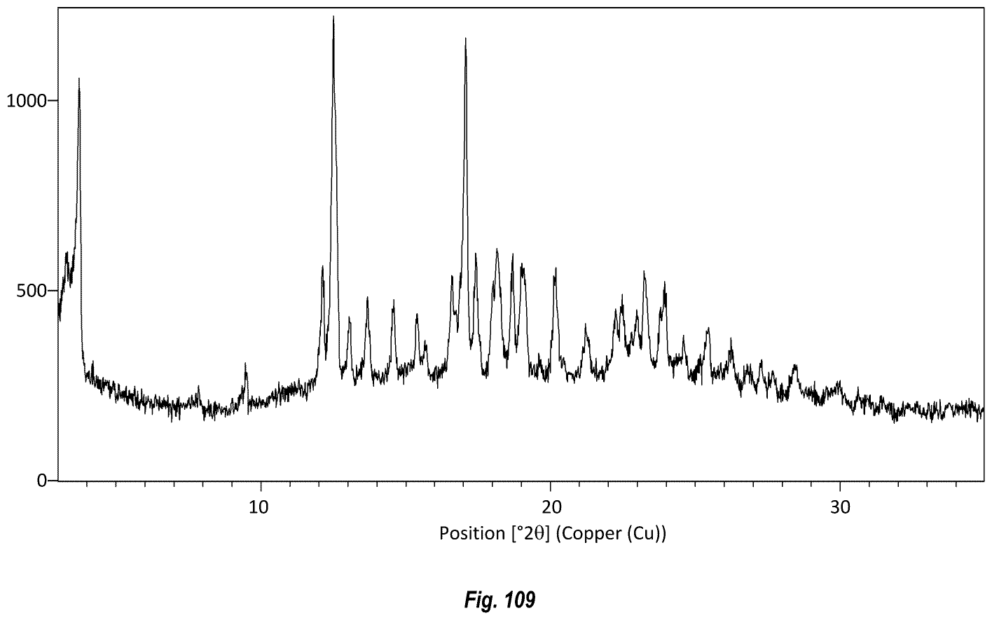

In some embodiments, the present disclosure relates to crystalline Compound 1 hydrogen phosphate and hydrates and solvates thereof. In some embodiments, the present disclosure relates to crystalline Compound 1 mono(hydrogen phosphate) and hydrates and solvates thereof. In some embodiments, the crystalline Compound 1 hydrogen phosphate or hydrate or solvate thereof or the crystalline Compound 1 mono(hydrogen phosphate) or hydrate or solvate thereof, including crystalline Form 1 Compound 1 hydrogen phosphate or a hydrate or solvate thereof, has an x-ray powder diffraction pattern comprising a diffraction pattern substantially the same as that of FIG. 109.

In some embodiments, the present disclosure relates to crystalline Form 1 Compound 1 hydrogen phosphate and hydrates and solvates thereof. In some embodiments, the crystalline Form 1 Compound 1 hydrogen chloride or a hydrate or solvate thereof is crystalline Form 1 Compound 1 di(hydrogen chloride) or a hydrate or solvate thereof.

In some embodiments, the crystalline Form 1 Compound 1 hydrogen phosphate or hydrate or solvate thereof has an X-ray powder diffraction pattern comprising one or more peaks chosen from the peaks listed in Table 20 below (all peaks that can be or have been chosen from Table 20 are rounded to the nearest 0.1° 2θ). In some embodiments, the crystalline Form 1 Compound 1 hydrogen phosphate or hydrate or solvate thereof has an X-ray powder diffraction pattern comprising two or more peaks chosen from the peaks listed in Table 20 below (all peaks that can be or have been chosen from Table 20 are rounded to the nearest 0.1° 2θ). In some embodiments, the crystalline Form 1 Compound 1 hydrogen phosphate or hydrate or solvate thereof has an X-ray powder diffraction pattern comprising three or more peaks chosen from the peaks listed in Table 20 below (all peaks that can be or have been chosen from Table 20 are rounded to the nearest 0.1° 2θ).

TABLE 20
Peak list of crystalline Form 1 Compound 1 hydrogen phosphate

| No. | Pos. [°2θ] | d-spacing [Å] | Height [cts] | Rel. Int. [%] |
|---|---|---|---|---|
| 1 | 3.2707 | 27.01405 | 347.61 | 32.97 |
| 2 | 3.7376 | 23.64057 | 816.88 | 77.48 |
| 3 | 7.662 | 11.53854 | 17.64 | 1.67 |
| 4 | 9.4979 | 9.31195 | 94.76 | 8.99 |
| 5 | 12.1193 | 7.30301 | 379.76 | 36.02 |
| 6 | 12.5086 | 7.0766 | 1054.26 | 100 |
| 7 | 13.0601 | 6.77899 | 243.34 | 23.08 |
| 8 | 13.6722 | 6.47683 | 284.83 | 27.02 |
| 9 | 14.5692 | 6.08005 | 278.62 | 26.43 |
| 10 | 15.3927 | 5.75659 | 251.29 | 23.84 |
| 11 | 16.5897 | 5.34382 | 351.64 | 33.35 |
| 12 | 17.0795 | 5.19164 | 975.79 | 92.56 |
| 13 | 17.4258 | 5.08925 | 413.52 | 39.22 |
| 14 | 18.1525 | 4.88713 | 425.38 | 40.35 |
| 15 | 18.6863 | 4.74871 | 398.25 | 37.77 |
| 16 | 19.0087 | 4.66889 | 378.15 | 35.87 |
| 17 | 20.1498 | 4.40697 | 348.65 | 33.07 |
| 18 | 21.2415 | 4.1829 | 198.69 | 18.85 |
| 19 | 22.2178 | 4.00125 | 233.06 | 22.11 |
| 20 | 22.4608 | 3.95851 | 275.38 | 26.12 |
| 21 | 23.2404 | 3.82745 | 360.8 | 34.22 |
| 22 | 23.9622 | 3.71376 | 301.6 | 28.61 |
| 23 | 24.6203 | 3.61597 | 163.56 | 15.51 |
| 24 | 25.4576 | 3.4989 | 209.17 | 19.84 |
| 25 | 26.2449 | 3.3957 | 155.6 | 14.76 |
| 26 | 27.2767 | 3.26955 | 114.62 | 10.87 |
| 27 | 27.6911 | 3.22156 | 85.08 | 8.07 |
| 28 | 28.4486 | 3.13748 | 106.12 | 10.07 |
| 29 | 29.9786 | 2.98075 | 52.29 | 4.96 |

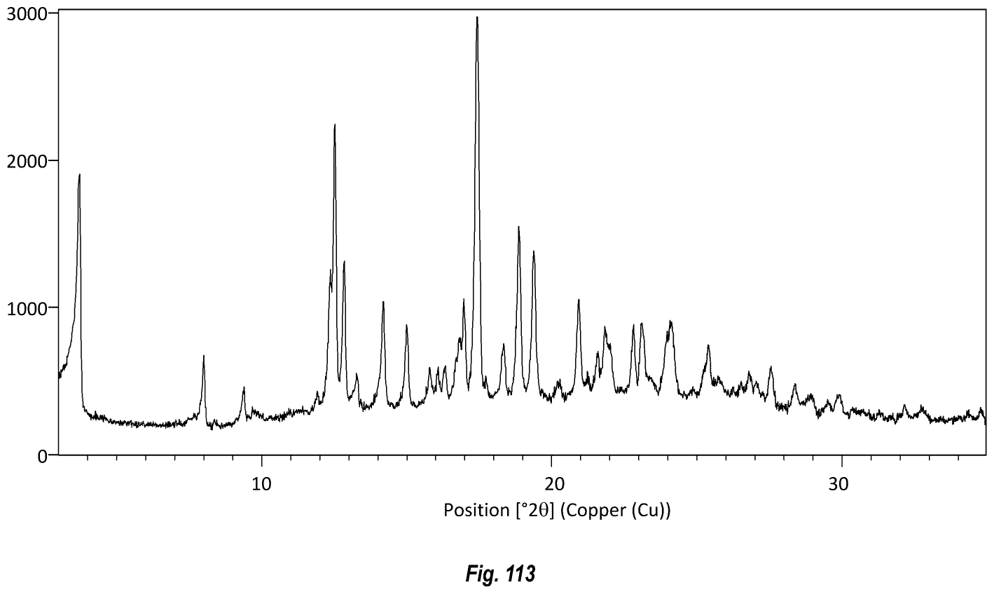

In some embodiments, the present disclosure relates to crystalline Compound 1 hydrogen phosphate and hydrates and solvates thereof and crystalline Compound 1 mono(hydrogen phosphate) and hydrates and solvates thereof, including crystalline Form 2 Compound 1 hydrogen phosphate or a hydrate or solvate thereof, having an X-ray powder diffraction pattern comprising a diffraction pattern substantially the same as that of FIG. 113.

In some embodiments, the present disclosure relates to crystalline Form 2 Compound 1 hydrogen phosphate and hydrates and solvates thereof. In some embodiments, the crystalline Form 2 Compound 1 hydrogen phosphate or a hydrate or solvate thereof is crystalline Form 2 Compound 1 mono(hydrogen phosphate) or a hydrate or solvate thereof.

In some embodiments, the crystalline Form 2 Compound 1 hydrogen phosphate or hydrate or solvate thereof has an X-ray powder diffraction pattern comprising one or more peaks chosen from the peaks listed in Table 21 below (all peaks that can be or have been chosen from Table 21 are rounded to the nearest 0.1° 2θ). In some embodiments, the crystalline Form 2 Compound 1 hydrogen phosphate or hydrate or solvate thereof has an X-ray powder diffraction pattern comprising two or more peaks chosen from the peaks listed in Table 21 below (all peaks that can be or have been chosen from Table 21 are rounded to the nearest 0.1° 2θ). In some embodiments, the crystalline Form 2 Compound 1 hydrogen phosphate or hydrate or solvate thereof has an X-ray powder diffraction pattern comprising three or more peaks chosen from the peaks listed in Table 21 below (all peaks that can be or have been chosen from Table 21 are rounded to the nearest 0.1° 2θ).

TABLE 21
Peak list of crystalline Form 2 Compound 1 hydrogen phosphate

| No. | Pos. [° 2θ] | d-spacing [Å] | Height [cts] | Rel. Int. [%] |
|---|---|---|---|---|
| 1 | 3.732 | 23.67608 | 1652.2 | 58.83 |
| 2 | 8.0092 | 11.03915 | 443.22 | 15.78 |
| 3 | 9.3944 | 9.41437 | 258.46 | 9.2 |
| 4 | 11.9189 | 7.4254 | 214.41 | 7.63 |
| 5 | 12.3501 | 7.16706 | 972.59 | 34.63 |
| 6 | 12.5297 | 7.06477 | 2073.2 | 73.82 |
| 7 | 12.8549 | 6.88674 | 1105.17 | 39.35 |
| 8 | 13.2864 | 6.66403 | 332.36 | 11.83 |
| 9 | 14.1891 | 6.24205 | 846.88 | 30.15 |
| 10 | 15.0095 | 5.90264 | 657.82 | 23.42 |
| 11 | 15.795 | 5.61084 | 375.61 | 13.37 |
| 12 | 16.0741 | 5.51403 | 359.82 | 12.81 |
| 13 | 16.3182 | 5.43209 | 393.77 | 14.02 |
| 14 | 16.9693 | 5.22511 | 863.01 | 30.73 |
| 15 | 17.4195 | 5.09109 | 2808.56 | 100 |
| 16 | 18.3397 | 4.83765 | 541.63 | 19.29 |
| 17 | 18.8674 | 4.70351 | 1364.69 | 48.59 |
| 18 | 19.3656 | 4.58362 | 1162 | 41.37 |
| 19 | 20.238 | 4.38797 | 268.56 | 9.56 |
| 20 | 20.9372 | 4.243 | 848.01 | 30.19 |
| 21 | 21.6074 | 4.11288 | 464.32 | 16.53 |
| 22 | 21.8316 | 4.07115 | 657.26 | 23.4 |
| 23 | 22.8061 | 3.89934 | 644.55 | 22.95 |
| 24 | 23.0991 | 3.85053 | 674.47 | 24.01 |
| 25 | 24.1197 | 3.68987 | 677 | 24.1 |
| 26 | 25.4318 | 3.5024 | 478.26 | 17.03 |
| 27 | 26.8015 | 3.32643 | 342.4 | 12.19 |
| 28 | 27.5696 | 3.23549 | 351.43 | 12.51 |
| 29 | 28.3733 | 3.14564 | 257.05 | 9.15 |
| 30 | 28.9414 | 3.08517 | 170.47 | 6.07 |
| 31 | 29.5369 | 3.02431 | 124.37 | 4.43 |
| 32 | 29.8951 | 2.98889 | 174.13 | 6.2 |
| 33 | 32.1716 | 2.7824 | 93.12 | 3.32 |
| 34 | 32.7729 | 2.73271 | 75 | 2.67 |

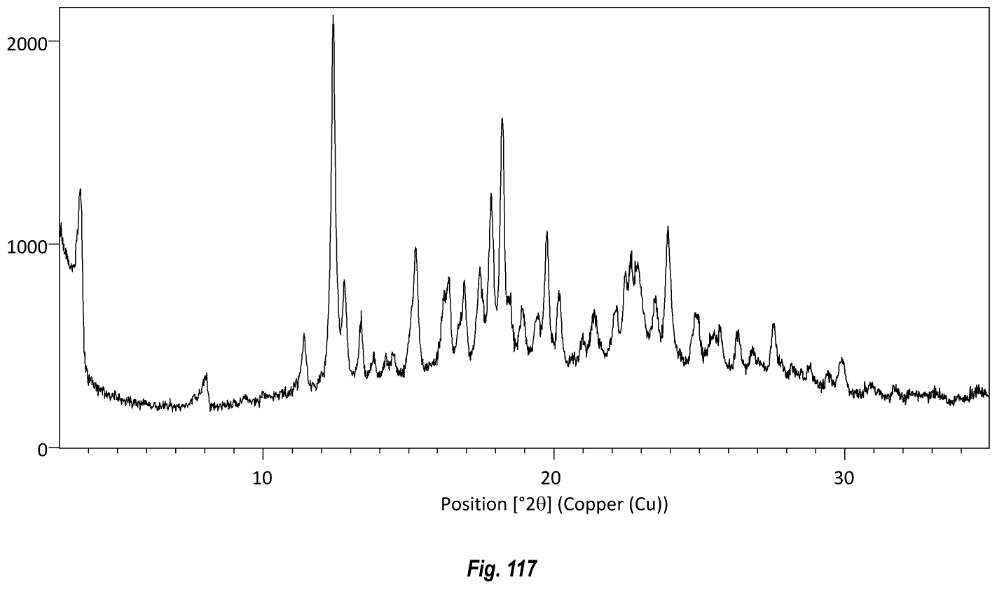

In some embodiments, the present disclosure relates to crystalline Compound 1 hydrogen phosphate and hydrates and solvates thereof and crystalline Compound 1 mono(hydrogen phosphate) and hydrates and solvates thereof, including crystalline Form 3 Compound 1 hydrogen phosphate or a hydrate or solvate thereof, having an X-ray powder diffraction pattern comprising a diffraction pattern substantially the same as that of FIG. 117.

In some embodiments, the present disclosure relates to crystalline Form 3 Compound 1 hydrogen phosphate and hydrates and solvates thereof. In some embodiments, the crystalline Form 3 Compound 1 hydrogen phosphate or a hydrate or solvate thereof is crystalline Form 3 Compound 1 mono(hydrogen phosphate) or a hydrate or solvate thereof.

In some embodiments, the crystalline Form 3 Compound 1 hydrogen phosphate or hydrate or solvate thereof has an X-ray powder diffraction pattern comprising one or more peaks chosen from the peaks listed in Table 22 below (all peaks that can be or have been chosen from Table 22 are rounded to the nearest 0.1° 2θ). In some embodiments, the crystalline Form 3 Compound 1 hydrogen phosphate or hydrate or solvate thereof has an X-ray powder diffraction pattern comprising two or more peaks chosen from the peaks listed in Table 22 below (all peaks that can be or have been chosen from Table 22 are rounded to the nearest 0.1° 2θ). In some embodiments, the crystalline Form 3 Compound 1 hydrogen phosphate or hydrate or solvate thereof has an X-ray powder diffraction pattern comprising three or more peaks chosen from the peaks listed in Table 22 below (all peaks that can be or have been chosen from Table 22 are rounded to the nearest 0.1° 2θ).

TABLE 22
Peak list of crystalline Form 3 Compound 1 hydrogen phosphate

| No. | Pos. [° 2θ] | d-spacing [Å] | Height [cts] | Rel. Int. [%] |
|---|---|---|---|---|
| 1 | 3.7548 | 23.53197 | 960.97 | 50.87 |
| 2 | 8.048 | 10.98597 | 147.48 | 7.81 |
| 3 | 11.415 | 7.75203 | 309.82 | 16.4 |
| 4 | 12.4131 | 7.13083 | 1889.15 | 100 |
| 5 | 12.8095 | 6.91107 | 561.72 | 29.73 |
| 6 | 13.3629 | 6.62606 | 378.48 | 20.03 |
| 7 | 13.7899 | 6.42184 | 194.02 | 10.27 |
| 8 | 14.3652 | 6.16592 | 167.86 | 8.89 |
| 9 | 15.2333 | 5.81644 | 743.7 | 39.37 |
| 10 | 16.4071 | 5.40287 | 599.61 | 31.74 |
| 11 | 16.9263 | 5.23828 | 560.66 | 29.68 |
| 12 | 17.4469 | 5.08315 | 628.63 | 33.28 |
| 13 | 17.8468 | 4.97013 | 1008.18 | 53.37 |
| 14 | 18.2527 | 4.86052 | 1306.11 | 69.14 |
| 15 | 18.9237 | 4.68965 | 428.75 | 22.7 |
| 16 | 19.4462 | 4.56481 | 396.01 | 20.96 |
| 17 | 19.7745 | 4.48975 | 781.22 | 41.35 |
| 18 | 20.1785 | 4.40079 | 503.67 | 26.66 |
| 19 | 20.9699 | 4.23646 | 279.99 | 14.82 |
| 20 | 21.372 | 4.15763 | 404.78 | 21.43 |
| 21 | 22.1504 | 4.01327 | 439.87 | 23.28 |
| 22 | 22.4632 | 3.95808 | 609.56 | 32.27 |
| 23 | 22.6413 | 3.92735 | 698.27 | 36.96 |
| 24 | 22.8928 | 3.88477 | 655.13 | 34.68 |
| 25 | 23.4708 | 3.79039 | 480.76 | 25.45 |
| 26 | 23.9085 | 3.72198 | 814.26 | 43.1 |
| 27 | 24.954 | 3.56836 | 389.46 | 20.62 |
| 28 | 25.7103 | 3.46508 | 340.24 | 18.01 |
| 29 | 26.3042 | 3.38819 | 301.99 | 15.99 |
| 30 | 26.8236 | 3.32375 | 223.73 | 11.84 |
| 31 | 27.5495 | 3.2378 | 356.44 | 18.87 |
| 32 | 29.4329 | 3.03476 | 105.39 | 5.58 |
| 33 | 29.9437 | 2.98414 | 166.6 | 8.82 |
| 34 | 30.8932 | 2.89456 | 48.48 | 2.57 |

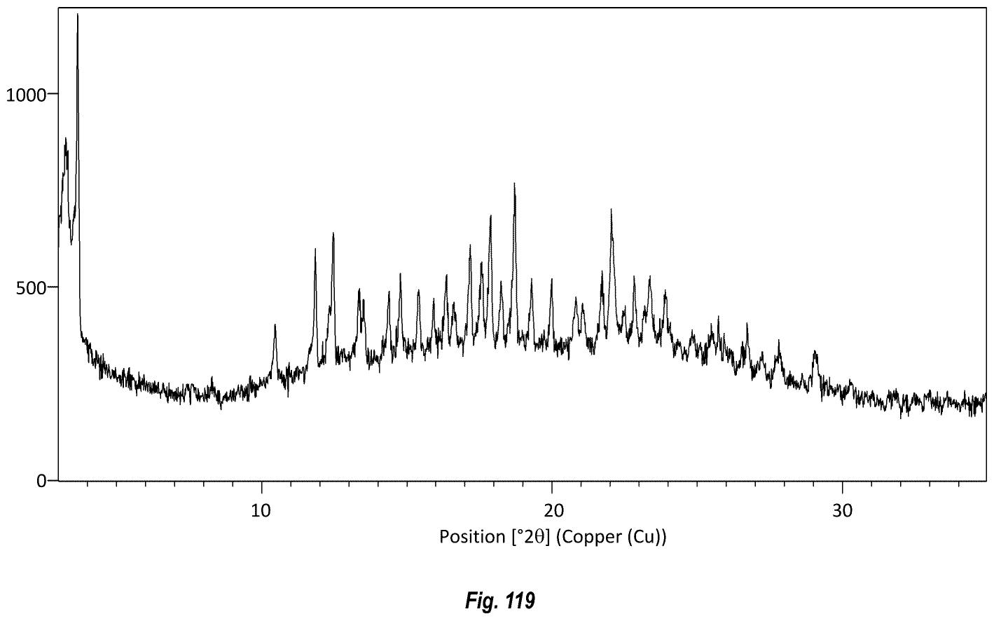

In some embodiments, the present disclosure relates to crystalline Compound 1 hydrogen phosphate and hydrates and solvates thereof and crystalline Compound 1 mono(hydrogen phosphate) and hydrates and solvates thereof, including crystalline Form 4 Compound 1 hydrogen phosphate or a hydrate or solvate thereof, having an X-ray powder diffraction pattern comprising a diffraction pattern substantially the same as that of FIG. 119.

In some embodiments, the present disclosure relates to crystalline Form 4 Compound 1 hydrogen phosphate and hydrates and solvates thereof. In some embodiments, the crystalline Form 4 Compound 1 hydrogen phosphate or a hydrate or solvate thereof is crystalline Form 4 Compound 1 mono(hydrogen phosphate) or a hydrate or solvate thereof.

In some embodiments, the crystalline Form 4 Compound 1 hydrogen phosphate or hydrate or solvate thereof has an X-ray powder diffraction pattern comprising one or more peaks chosen from the peaks listed in Table 23 below (all peaks that can be or have been chosen from Table 23 are rounded to the nearest 0.1° 2θ). In some embodiments, the crystalline Form 4 Compound 1 hydrogen phosphate or hydrate or solvate thereof has an X-ray powder diffraction pattern comprising two or more peaks chosen from the peaks listed in Table 23 below (all peaks that can be or have been chosen from Table 23 are rounded to the nearest 0.1° 2θ). In some embodiments, the crystalline Form 4 Compound 1 hydrogen phosphate or hydrate or solvate thereof has an X-ray powder diffraction pattern comprising three or more peaks chosen from the peaks listed in Table 23 below (all peaks that can be or have been chosen from Table 23 are rounded to the nearest 0.1° 2θ).

TABLE 23
Peak list of crystalline Form 4 Compound 1 hydrogen phosphate

| No. | Pos. [° 2θ] | d-spacing [Å] | Height [cts] | Rel. Int. [%] |
|---|---|---|---|---|
| 1 | 3.2764 | 26.96683 | 588.04 | 61.99 |
| 2 | 3.6701 | 24.07489 | 948.53 | 100 |
| 3 | 10.4709 | 8.44876 | 176.72 | 18.63 |
| 4 | 11.8507 | 7.46799 | 384.25 | 40.51 |
| 5 | 12.4705 | 7.09818 | 425.08 | 44.81 |
| 6 | 13.3533 | 6.63081 | 262.37 | 27.66 |
| 7 | 13.5228 | 6.54809 | 237.61 | 25.05 |
| 8 | 14.3845 | 6.15768 | 261.38 | 27.56 |
| 9 | 14.7851 | 5.99173 | 316.81 | 33.4 |
| 10 | 15.4093 | 5.75039 | 258.84 | 27.29 |
| 11 | 15.9141 | 5.56913 | 231.68 | 24.43 |
| 12 | 16.365 | 5.41667 | 316.79 | 33.4 |
| 13 | 16.6301 | 5.33094 | 218.67 | 23.05 |
| 14 | 17.1816 | 5.16102 | 390.22 | 41.14 |
| 15 | 17.5798 | 5.04502 | 335.35 | 35.36 |
| 16 | 17.894 | 4.95712 | 465.07 | 49.03 |
| 17 | 18.2645 | 4.85741 | 271.47 | 28.62 |
| 18 | 18.724 | 4.73922 | 523.96 | 55.24 |
| 19 | 19.3059 | 4.59767 | 290.68 | 30.65 |
| 20 | 19.9831 | 4.44336 | 284.17 | 29.96 |
| 21 | 20.8335 | 4.26387 | 243.64 | 25.69 |
| 22 | 21.7219 | 4.09145 | 294.83 | 31.08 |
| 23 | 22.0482 | 4.03163 | 456.88 | 48.17 |
| 24 | 22.8383 | 3.89392 | 298.86 | 31.51 |
| 25 | 23.3731 | 3.80602 | 296.41 | 31.25 |
| 26 | 23.9074 | 3.72216 | 260.54 | 27.47 |
| 27 | 24.8676 | 3.58057 | 143.29 | 15.11 |
| 28 | 25.5848 | 3.4818 | 133.51 | 14.08 |
| 29 | 26.7456 | 3.33326 | 141.49 | 14.92 |
| 30 | 27.8082 | 3.20826 | 106.51 | 11.23 |
| 31 | 29.0284 | 3.07612 | 102.23 | 10.78 |

In some embodiments, the present disclosure relates to crystalline Compound 1 hydrogen phosphate and hydrates and solvates thereof and crystalline Compound 1 mono(hydrogen phosphate) and hydrates and solvates thereof, including crystalline Form 4 Compound 1 hydrogen phosphate or a hydrate or solvate thereof, having an X-ray powder diffraction pattern comprising a peak at about 3.7° 2θ.

In some embodiments, the present disclosure relates to crystalline Compound 1 di(hydrogen phosphate) and hydrates and solvates thereof. In many embodiments, the crystalline Compound 1 di(hydrogen phosphate) or hydrate or solvate thereof is a di(dihydrogen phosphate) or hydrate or solvate thereof.

In some embodiments, the present disclosure relates to crystalline Compound 1 hydrogen phosphate and hydrates and solvates thereof and crystalline Compound 1 di(hydrogen phosphate) and hydrates and solvates thereof, including crystalline Form 5 Compound 1 hydrogen phosphate or a hydrate or solvate thereof, having an X-ray powder diffraction pattern comprising a peak at about 4.0° 2θ. In some embodiments, the crystalline Compound 1 hydrogen phosphate or hydrate or solvate thereof or the crystalline Compound 1 di(hydrogen phosphate) or hydrate or solvate thereof, including crystalline Form 5 Compound 1 hydrogen phosphate or a hydrate or solvate thereof, has an X-ray powder diffraction pattern comprising peaks at about 4.0° 2θ and about 8.2° 2θ. In some embodiments, the crystalline Compound 1 hydrogen phosphate or hydrate or solvate thereof or the crystalline Compound 1 di(hydrogen phosphate) or hydrate or solvate thereof, including crystalline Form 5 Compound 1 hydrogen phosphate or a hydrate or solvate thereof, has an X-ray powder diffraction pattern comprising peaks at about 4.0° 2θ, about 8.2° 2θ, and about 9.7° 2θ. In some embodiments, the crystalline Compound 1 hydrogen phosphate or hydrate or solvate thereof or the crystalline Compound 1 di(hydrogen phosphate) or hydrate or solvate thereof, including crystalline Form 5 Compound 1 hydrogen phosphate or a hydrate or solvate thereof, has an X-ray powder diffraction pattern substantially the same as that of FIG. 121.

In some embodiments, the present disclosure relates to crystalline Form 5 Compound 1 hydrogen phosphate and hydrates and solvates thereof. In some embodiments, the crystalline Form 5 Compound 1 hydrogen phosphate or hydrate or solvate thereof is crystalline Form 5 Compound 1 di(hydrogen phosphate) or a hydrate or solvate thereof.

In some embodiments, the crystalline Form 5 Compound 1 hydrogen phosphate or hydrate or solvate thereof has an X-ray powder diffraction pattern comprising one or more peaks chosen from the peaks listed in Table 24 below (all peaks that can be or have been chosen from Table 24 are rounded to the nearest 0.1° 2θ). In some embodiments, the crystalline Form 5 Compound 1 hydrogen phosphate or hydrate or solvate thereof has an X-ray powder diffraction pattern comprising two or more peaks chosen from the peaks listed in Table 24 below (all peaks that can be or have been chosen from Table 24 are rounded to the nearest 0.1° 2θ). In some embodiments, the crystalline Form 5 Compound 1 hydrogen phosphate or hydrate or solvate thereof has an X-ray powder diffraction pattern comprising three or more peaks chosen from the peaks listed in Table 24 below (all peaks that can be or have been chosen from Table 24 are rounded to the nearest 0.1° 2θ).

TABLE 24
Peak list of crystalline Form 5 Compound 1 hydrogen phosphate

| No. | Pos. [° 2θ] | d-spacing [Å] | Height [cts] | Rel. Int. [%] |
|---|---|---|---|---|
| 1 | 3.98 | 22.20144 | 1555.1 | 72.06 |
| 2 | 4.8903 | 18.07043 | 77.07 | 3.57 |
| 3 | 8.1833 | 10.80474 | 252.99 | 11.72 |
| 4 | 9.7298 | 9.09051 | 484.79 | 22.46 |
| 5 | 10.3041 | 8.58513 | 190.14 | 8.81 |
| 6 | 10.7143 | 8.25739 | 147.63 | 6.84 |
| 7 | 11.4056 | 7.75839 | 244.02 | 11.31 |
| 8 | 12.2962 | 7.19836 | 811.89 | 37.62 |
| 9 | 12.7354 | 6.95109 | 304.77 | 14.12 |
| 10 | 13.2715 | 6.67151 | 642.16 | 29.75 |
| 11 | 13.9231 | 6.36071 | 842.39 | 39.03 |
| 12 | 14.3691 | 6.16425 | 304.1 | 14.09 |
| 13 | 15.161 | 5.84403 | 289.72 | 13.42 |
| 14 | 16.0069 | 5.53704 | 910.44 | 42.19 |
| 15 | 16.186 | 5.47618 | 897.12 | 41.57 |
| 16 | 16.4308 | 5.39514 | 698.68 | 32.37 |
| 17 | 16.9374 | 5.2349 | 621.2 | 28.78 |
| 18 | 17.5873 | 5.04289 | 404.79 | 18.76 |
| 19 | 18.1885 | 4.87753 | 1107.16 | 51.3 |
| 20 | 18.5581 | 4.78121 | 392.22 | 18.17 |
| 21 | 19.0577 | 4.65698 | 1039.98 | 48.19 |
| 22 | 20.6919 | 4.29273 | 2158.2 | 100 |
| 23 | 21.2066 | 4.1897 | 1278.59 | 59.24 |
| 24 | 21.6867 | 4.09802 | 1551.97 | 71.91 |
| 25 | 22.544 | 3.94409 | 393.37 | 18.23 |
| 26 | 22.9274 | 3.87899 | 654.85 | 30.34 |
| 27 | 23.5886 | 3.77173 | 641.55 | 29.73 |
| 28 | 24.2596 | 3.6689 | 628.7 | 29.13 |
| 29 | 24.6634 | 3.60974 | 558.76 | 25.89 |
| 30 | 25.2317 | 3.52972 | 561.15 | 26 |
| 31 | 25.6923 | 3.46748 | 791.37 | 36.67 |
| 32 | 26.7674 | 3.3306 | 305.62 | 14.16 |
| 33 | 27.0656 | 3.29457 | 254.41 | 11.79 |
| 34 | 27.8491 | 3.20364 | 380.16 | 17.61 |
| 35 | 28.288 | 3.15493 | 348.02 | 16.13 |
| 36 | 30.1808 | 2.96124 | 201.93 | 9.36 |
| 37 | 30.6138 | 2.92034 | 162.17 | 7.51 |
| 38 | 31.2981 | 2.85803 | 152.84 | 7.08 |
| 39 | 32.0007 | 2.79687 | 201.9 | 9.36 |
| 40 | 33.1566 | 2.70196 | 169.12 | 7.84 |
| 41 | 33.7209 | 2.65803 | 153.8 | 7.13 |
| 42 | 34.328 | 2.61239 | 118.85 | 5.51 |

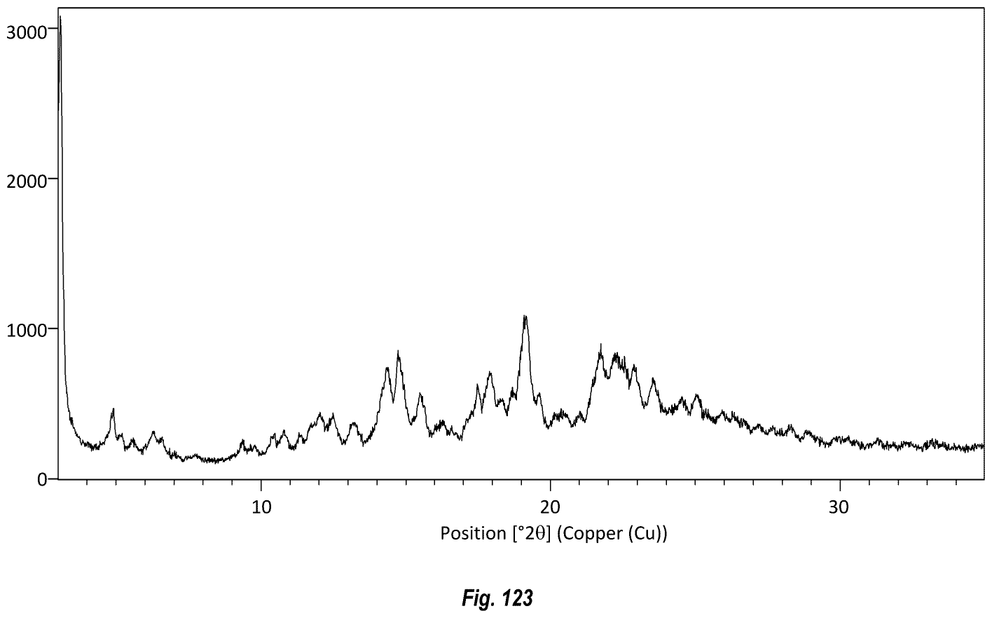

In some embodiments, the present disclosure relates to crystalline Compound 1 hydrogen phosphate and hydrates and solvates thereof and crystalline Compound 1 di(hydrogen phosphate) and hydrates and solvates thereof, including crystalline Form 6 Compound 1 hydrogen phosphate or a hydrate or solvate thereof, having an X-ray powder diffraction pattern comprising a peak at about 3.1° 2θ. In some embodiments, the crystalline Compound 1 hydrogen phosphate or hydrate or solvate thereof or the crystalline Compound 1 di(hydrogen phosphate) or hydrate or solvate thereof, including crystalline Form 6 Compound 1 hydrogen phosphate or a hydrate or solvate thereof, has an X-ray powder diffraction pattern comprising peaks at about 3.1° 2θ and about 5.6° 2θ. In some embodiments, the crystalline Compound 1 hydrogen phosphate or hydrate or solvate thereof or the crystalline Compound 1 di(hydrogen phosphate) or hydrate or solvate thereof, including crystalline Form 6 Compound 1 hydrogen phosphate or a hydrate or solvate thereof, has an X-ray powder diffraction pattern substantially the same as that of FIG. 123.

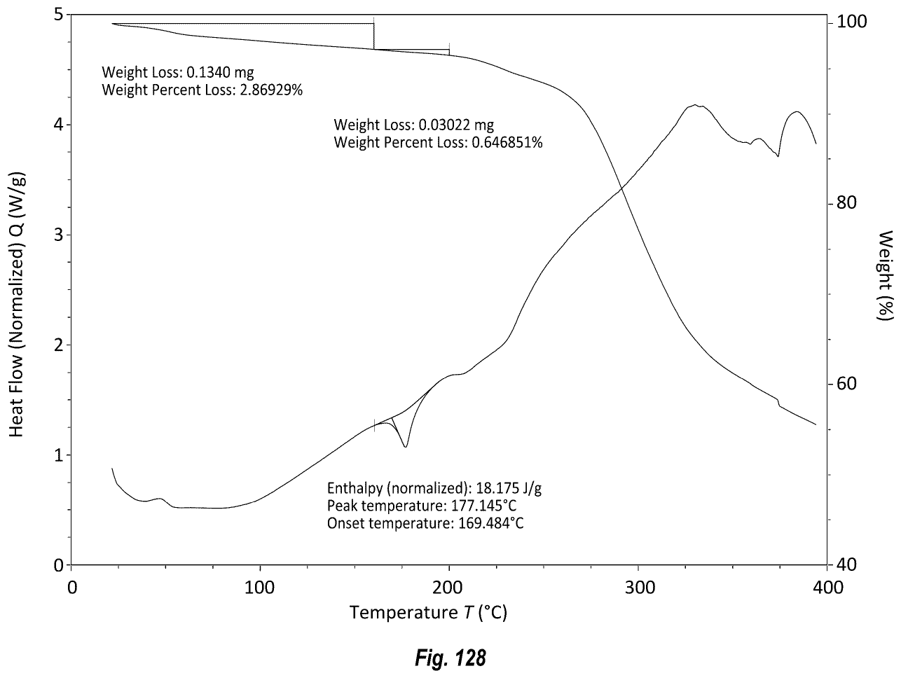

In some embodiments, the crystalline Compound 1 hydrogen phosphate or hydrate or solvate thereof or the crystalline Compound 1 di(hydrogen phosphate) or hydrate or solvate thereof, including crystalline Form 6 Compound 1 hydrogen phosphate or a hydrate or solvate thereof, has a DSC endotherm with an onset of about 141° C. In some embodiments, the endotherm is a melting endotherm. In some embodiments, the crystalline Compound 1 hydrogen phosphate or hydrate or solvate thereof or the crystalline Compound 1 di(hydrogen phosphate) or hydrate or solvate thereof, including crystalline Form 6 Compound 1 hydrogen phosphate or a hydrate or solvate thereof, has a DSC thermogram substantially the same as that of FIG. 128.

In some embodiments, the present disclosure relates to crystalline Form 6 Compound 1 hydrogen phosphate and hydrates and solvates thereof. In some embodiments, the crystalline Form 6 Compound 1 hydrogen phosphate or hydrate or solvate thereof is crystalline Form 6 Compound 1 di(hydrogen phosphate) or hydrate or solvate thereof.

In some embodiments, the crystalline Form 6 Compound 1 hydrogen phosphate or hydrate or solvate thereof has an X-ray powder diffraction pattern comprising one or more peaks chosen from the peaks listed in Table 25 below (all peaks that can be or have been chosen from Table 25 are rounded to the nearest 0.1° 2θ). In some embodiments, the crystalline Form 6 Compound 1 hydrogen phosphate or hydrate or solvate thereof has an X-ray powder diffraction pattern comprising two or more peaks chosen from the peaks listed in Table 25 below (all peaks that can be or have been chosen from Table 25 are rounded to the nearest 0.1° 2θ). In some embodiments, the crystalline Form 6 Compound 1 hydrogen phosphate or hydrate or solvate thereof has an X-ray powder diffraction pattern comprising three or more peaks chosen from the peaks listed in Table 25 below (all peaks that can be or have been chosen from Table 25 are rounded to the nearest 0.1° 2θ).

TABLE 25
Peak list of crystalline Form 6 Compound 1 hydrogen phosphate

| No. | Pos. [° 2θ] | d-spacing [Å] | Height [cts] | Rel. Int. [%] |
|---|---|---|---|---|
| 1 | 3.0981 | 28.51892 | 2943.46 | 100 |
| 2 | 4.9001 | 18.03439 | 341.16 | 11.59 |
| 3 | 5.5878 | 15.81628 | 128.96 | 4.38 |
| 4 | 6.2666 | 14.10452 | 188.13 | 6.39 |
| 5 | 7.6962 | 11.48734 | 27.37 | 0.93 |
| 6 | 9.3455 | 9.46345 | 105.72 | 3.59 |
| 7 | 10.4673 | 8.45161 | 147.41 | 5.01 |
| 8 | 10.8114 | 8.18338 | 151.07 | 5.13 |
| 9 | 11.3434 | 7.80076 | 118.6 | 4.03 |
| 10 | 12.0514 | 7.34403 | 249.48 | 8.48 |
| 11 | 12.4921 | 7.08592 | 236.23 | 8.03 |
| 12 | 13.1905 | 6.71225 | 178.91 | 6.08 |
| 13 | 14.3591 | 6.16851 | 529.25 | 17.98 |
| 14 | 14.7372 | 6.01112 | 621.87 | 21.13 |
| 15 | 15.4729 | 5.72692 | 327.7 | 11.13 |
| 16 | 16.2808 | 5.4445 | 132.35 | 4.5 |
| 17 | 17.4915 | 5.07027 | 364.43 | 12.38 |
| 18 | 17.9225 | 4.94932 | 439.31 | 14.92 |
| 19 | 19.2013 | 4.62249 | 744.14 | 25.28 |
| 20 | 19.6014 | 4.52901 | 271.59 | 9.23 |
| 21 | 20.3711 | 4.35961 | 124.09 | 4.22 |
| 22 | 21.7112 | 4.09345 | 516.73 | 17.56 |
| 23 | 22.2163 | 4.00151 | 490.29 | 16.66 |
| 24 | 22.8525 | 3.89153 | 404.31 | 13.74 |
| 25 | 23.5287 | 3.7812 | 306.31 | 10.41 |
| 26 | 24.5313 | 3.62888 | 165.94 | 5.64 |
| 27 | 25.0663 | 3.55264 | 179.25 | 6.09 |
| 28 | 28.3073 | 3.15282 | 53.14 | 1.81 |
| 29 | 28.8582 | 3.09387 | 45.43 | 1.54 |
| 30 | 31.2683 | 2.86068 | 31.74 | 1.08 |
| 31 | 32.208 | 2.77933 | 21.61 | 0.73 |

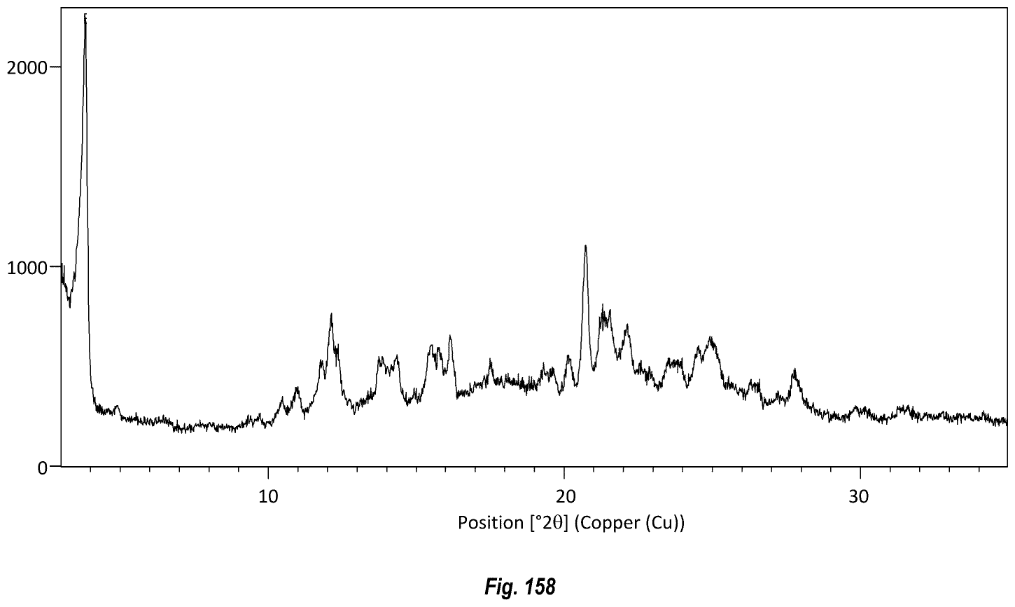

In some embodiments, the present disclosure relates to crystalline Compound 1 hydrogen phosphate and hydrates and solvates thereof and crystalline Compound 1 di(hydrogen phosphate) and hydrates and solvates thereof, including crystalline Form 7 Compound 1 hydrogen phosphate or a hydrate and solvate thereof, having an X-ray powder diffraction pattern comprising a peak at about 3.6° 2θ. In some embodiments, the crystalline Compound 1 hydrogen phosphate or hydrate and solvate thereof or the crystalline Compound 1 di(hydrogen phosphate) or hydrate and solvate thereof, including crystalline Form 7 Compound 1 hydrogen phosphate or a hydrate and solvate thereof, has an X-ray powder diffraction pattern comprising one or more peaks at about 3.6° 2θ, about 12.1° 2θ, and about 20.1° 2θ. In some embodiments, the crystalline Compound 1 hydrogen phosphate or hydrate and solvate thereof or the crystalline Compound 1 di(hydrogen phosphate) or hydrate and solvate thereof, including crystalline Form 7 Compound 1 hydrogen phosphate or a hydrate and solvate thereof, has an X-ray powder diffraction pattern substantially the same as that of FIG. 158.

In some embodiments, the present disclosure relates to crystalline Form 7 Compound 1 hydrogen phosphate and hydrates and solvates thereof. In some embodiments, the crystalline Form 7 Compound 1 hydrogen phosphate or hydrate and solvate thereof is crystalline Form 7 Compound 1 di(hydrogen phosphate) or hydrate and solvate thereof.

In some embodiments, the crystalline Form 7 Compound 1 hydrogen phosphate or hydrate or solvate thereof has an X-ray powder diffraction pattern comprising one or more peaks chosen from the peaks listed in Table 26 below (all peaks that can be or have been chosen from Table 26 are rounded to the nearest 0.1° 2θ). In some embodiments, the crystalline Form 7 Compound 1 hydrogen phosphate or hydrate or solvate thereof has an X-ray powder diffraction pattern comprising two or more peaks chosen from the peaks listed in Table 26 below (all peaks that can be or have been chosen from Table 26 are rounded to the nearest 0.1° 2θ). In some embodiments, the crystalline Form 7 Compound 1 hydrogen phosphate or hydrate or solvate thereof has an X-ray powder diffraction pattern comprising three or more peaks chosen from the peaks listed in Table 26 below (all peaks that can be or have been chosen from Table 26 are rounded to the nearest 0.1° 2θ).

TABLE 26
Peak list of crystalline Form 7 Compound 1 hydrogen phosphate

| No. | Pos. [° 2θ] | d-spacing [Å] | Height [cts] | Rel. Int. [%] |
|---|---|---|---|---|
| 1 | 3.8372 | 23.02675 | 2047.8 | 100 |
| 2 | 4.8984 | 18.0407 | 80.95 | 3.95 |
| 3 | 10.4638 | 8.45446 | 123.55 | 6.03 |
| 4 | 10.9773 | 8.06008 | 179.46 | 8.76 |
| 5 | 11.7948 | 7.50322 | 302.68 | 14.78 |
| 6 | 12.1353 | 7.29346 | 544.03 | 26.57 |
| 7 | 13.722 | 6.45347 | 311.66 | 15.22 |
| 8 | 14.3869 | 6.15667 | 315.23 | 15.39 |
| 9 | 15.5088 | 5.71374 | 401.4 | 19.6 |
| 10 | 16.1563 | 5.48617 | 442.86 | 21.63 |
| 11 | 17.5048 | 5.06646 | 266.91 | 13.03 |
| 12 | 19.6381 | 4.52064 | 260.33 | 12.71 |
| 13 | 20.1287 | 4.41155 | 328.83 | 16.06 |
| 14 | 20.739 | 4.28309 | 888.16 | 43.37 |
| 15 | 21.2539 | 4.18047 | 523.88 | 25.58 |
| 16 | 21.5511 | 4.12349 | 534.44 | 26.1 |
| 17 | 22.1339 | 4.01621 | 470.11 | 22.96 |
| 18 | 23.9292 | 3.71881 | 282.01 | 13.77 |
| 19 | 24.4848 | 3.63567 | 349.88 | 17.09 |
| 20 | 24.9549 | 3.56825 | 405.59 | 19.81 |
| 21 | 26.4204 | 3.37355 | 171.49 | 8.37 |
| 22 | 27.7596 | 3.21377 | 231.6 | 11.31 |
| 23 | 29.8337 | 2.9949 | 49.12 | 2.4 |
| 24 | 31.4442 | 2.84508 | 43.09 | 2.1 |

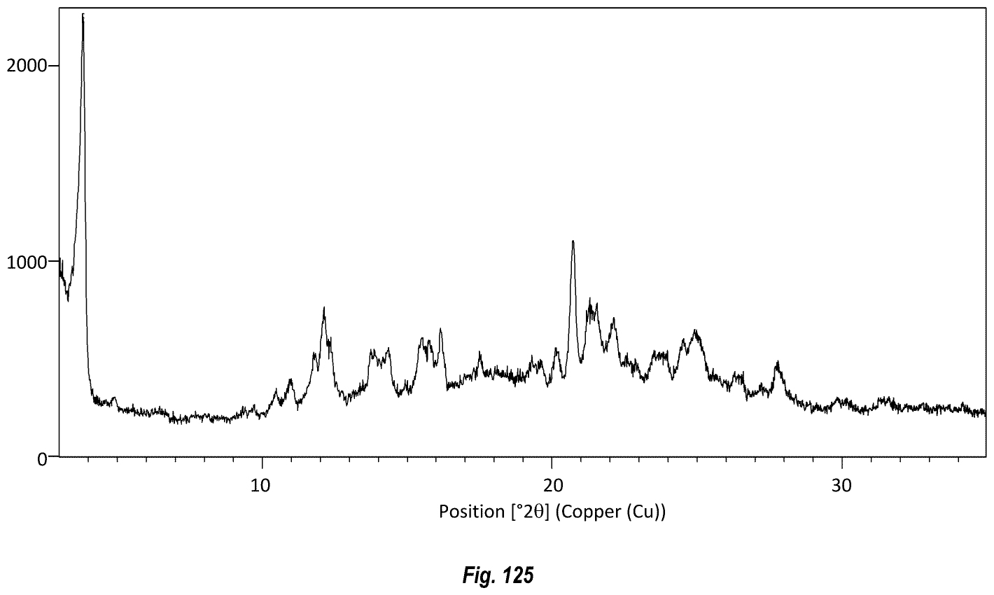

In some embodiments, the present disclosure relates to crystalline Compound 1 hydrogen phosphate and hydrates and solvates thereof and crystalline Compound 1 di(hydrogen phosphate) and hydrates and solvates thereof, including crystalline Form 8 Compound 1 or a hydrate or solvate thereof, having an X-ray powder diffraction pattern comprising a peak at about 3.8° 2θ. In some embodiments, the crystalline Compound 1 hydrogen phosphate or hydrate or solvate thereof or the crystalline Compound 1 di(hydrogen phosphate) or hydrate or solvate thereof, including crystalline Form 8 Compound 1 or a hydrate or solvate thereof, has an X-ray powder diffraction pattern comprising one or more peaks at about 3.8° 2θ and about 16.2° 2θ. In some embodiments, the crystalline Compound 1 hydrogen phosphate or hydrate or solvate thereof or the crystalline Compound 1 di(hydrogen phosphate) or hydrate or solvate thereof, including crystalline Form 8 Compound 1 or a hydrate or solvate thereof, has an X-ray powder diffraction pattern substantially the same as that of FIG. 125.

In some embodiments, the present disclosure relates to crystalline Form 8 Compound 1 hydrogen phosphate and hydrates and solvates thereof. In some embodiments, the crystalline Form 8 Compound 1 hydrogen phosphate or hydrate or solvate thereof is crystalline Form 8 Compound 1 di(hydrogen phosphate) or hydrate or solvate thereof.

In some embodiments, the crystalline Form 8 Compound 1 hydrogen phosphate or hydrate or solvate thereof has an X-ray powder diffraction pattern comprising one or more peaks chosen from the peaks listed in Table 27 below (all peaks that can be or have been chosen from Table 27 are rounded to the nearest 0.1° 2θ). In some embodiments, the crystalline Form 8 Compound 1 hydrogen phosphate or hydrate or solvate thereof has an X-ray powder diffraction pattern comprising two or more peaks chosen from the peaks listed in Table 27 below (all peaks that can be or have been chosen from Table 27 are rounded to the nearest 0.1° 2θ). In some embodiments, the crystalline Form 8 Compound 1 hydrogen phosphate or hydrate or solvate thereof has an X-ray powder diffraction pattern comprising three or more peaks chosen from the peaks listed in Table 27 below (all peaks that can be or have been chosen from Table 27 are rounded to the nearest 0.1° 2θ).

TABLE 27
Peak list of crystalline Form 8 Compound 1 hydrogen phosphate

| No. | Pos. [° 2θ] | d-spacing [Å] | Height [cts] | Rel. Int. [%] |
|---|---|---|---|---|
| 1 | 3.8372 | 23.02675 | 2047.8 | 100 |
| 2 | 4.8984 | 18.0407 | 80.95 | 3.95 |
| 3 | 10.4638 | 8.45446 | 123.55 | 6.03 |
| 4 | 10.9773 | 8.06008 | 179.46 | 8.76 |
| 5 | 11.7948 | 7.50322 | 302.68 | 14.78 |
| 6 | 12.1353 | 7.29346 | 544.03 | 26.57 |
| 7 | 13.722 | 6.45347 | 311.66 | 15.22 |
| 8 | 14.3869 | 6.15667 | 315.23 | 15.39 |
| 9 | 15.5088 | 5.71374 | 401.4 | 19.6 |
| 10 | 16.1563 | 5.48617 | 442.86 | 21.63 |
| 11 | 17.5048 | 5.06646 | 266.91 | 13.03 |
| 12 | 19.6381 | 4.52064 | 260.33 | 12.71 |
| 13 | 20.1287 | 4.41155 | 328.83 | 16.06 |
| 14 | 20.739 | 4.28309 | 888.16 | 43.37 |
| 15 | 21.2539 | 4.18047 | 523.88 | 25.58 |
| 16 | 21.5511 | 4.12349 | 534.44 | 26.1 |
| 17 | 22.1339 | 4.01621 | 470.11 | 22.96 |
| 18 | 23.9292 | 3.71881 | 282.01 | 13.77 |
| 19 | 24.4848 | 3.63567 | 349.88 | 17.09 |
| 20 | 24.9549 | 3.56825 | 405.59 | 19.81 |
| 21 | 26.4204 | 3.37355 | 171.49 | 8.37 |
| 22 | 27.7596 | 3.21377 | 231.6 | 11.31 |
| 23 | 29.8337 | 2.9949 | 49.12 | 2.4 |
| 24 | 31.4442 | 2.84508 | 43.09 | 2.1 |

In some embodiments, the present disclosure relates to crystalline Compound 1 hydrogen phosphate and hydrates and solvates thereof and crystalline Compound 1 di(hydrogen phosphate) and hydrates and solvates thereof, including crystalline Form 8 Compound 1 hydrogen phosphate or a hydrate or solvate thereof, having an X-ray powder diffraction pattern comprising peak at about 4.9° 2θ.

In one aspect, the present disclosure relates to Compound 1 fumarate and hydrates and solvates thereof. Compound 1 fumarate is formed by, for example, the combination of Compound 1 (free base) and fumaric acid and can be represented as shown below where n is a number greater than zero.

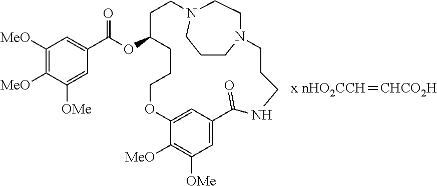

In some embodiments, the present disclosure relates to Compound 1 mono(fumarate) and hydrates and solvates thereof. Compound 1 mono(fumarate) is formed by, for example, the combination of Compound 1 (free base) and one molecular equivalent of fumaric acid and can be represented as shown below.

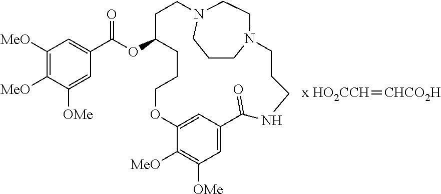

In some embodiments, the present disclosure relates to Compound 1 di(fumarate) and hydrates and solvates thereof. Compound 1 di(fumarate) is formed by, for example, the combination of Compound 1 (free base) and two molecular equivalents of fumaric acid and can be represented as shown below.

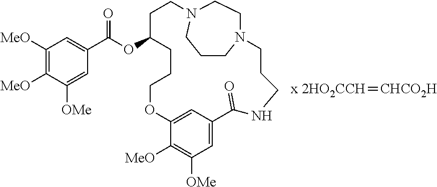

In some embodiments, the present disclosure relates to amorphous Compound 1 fumarate and hydrates and solvates thereof. In some embodiments, the present disclosure relates to amorphous Compound 1 mono(fumarate) and hydrates and solvates thereof. In some embodiments, the present disclosure relates to amorphous Compound 1 di(fumarate) and hydrates and solvates thereof.

In some embodiments, the present disclosure relates to crystalline Compound 1 fumarate and hydrates and solvates thereof. In some embodiments, the present disclosure relates to crystalline Compound 1 mono(fumarate) and hydrates and solvates thereof. In some embodiments, the present disclosure relates to crystalline Compound 1 di(fumarate) and hydrates and solvates thereof.

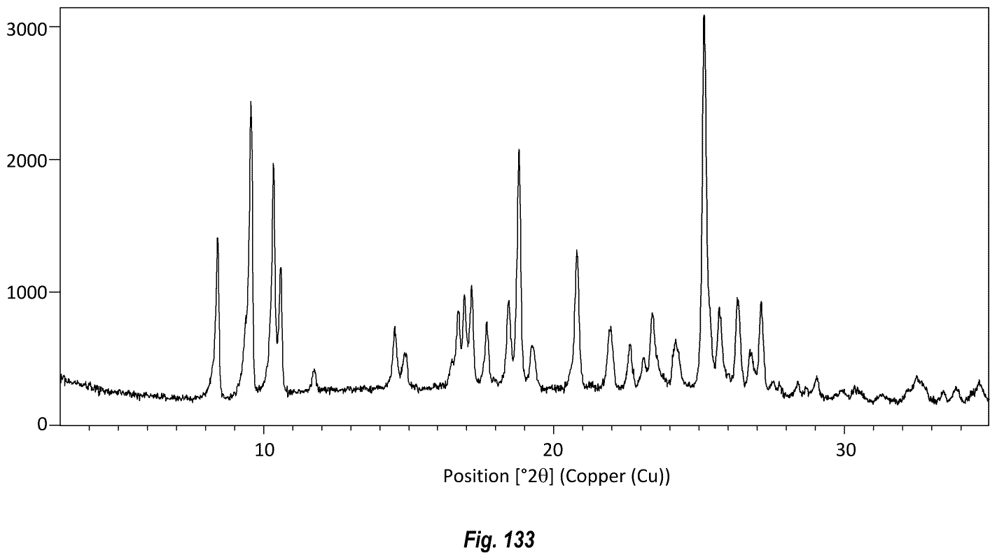

In some embodiments, the present disclosure relates to crystalline Compound 1 fumarate and hydrates and solvates thereof and crystalline Compound 1 mono(fumarate) and hydrates and solvates thereof, including crystalline Form 1 Compound 1 fumarate or a hydrate or solvate thereof, having an X-ray powder diffraction pattern comprising a peak at about 8.4° 2θ. In some embodiments, the crystalline Compound 1 fumarate or hydrate or solvate thereof or the crystalline Compound 1 mono(fumarate) or hydrate or solvate thereof, including crystalline Form 1 Compound 1 fumarate or a hydrate or solvate thereof, has an X-ray powder diffraction pattern comprising peaks at about 8.4° 2θ and about 14.5° 2θ. In some embodiments, the crystalline Compound 1 fumarate or hydrate or solvate thereof or the crystalline Compound 1 mono(fumarate) or hydrate or solvate thereof, including crystalline Form 1 Compound 1 fumarate or a hydrate or solvate thereof, has an X-ray powder diffraction pattern substantially the same as that of FIG. 133.

In some embodiments, the present disclosure relates to crystalline Form 1 Compound 1 fumarate and hydrates and solvates thereof. In some embodiments, the crystalline Form 1 Compound 1 fumarate or hydrate or solvate thereof is crystalline Form 1 Compound 1 mono(fumarate) or hydrate or solvate thereof.

In some embodiments, the crystalline Form 1 Compound 1 fumarate or hydrate or solvate thereof has an X-ray powder diffraction pattern comprising one or more peaks chosen from the peaks listed in Table 28 below (all peaks that can be or have been chosen from Table 28 are rounded to the nearest 0.1° 2θ). In some embodiments, the crystalline Form 1 Compound 1 fumarate or hydrate or solvate thereof has an X-ray powder diffraction pattern comprising two or more peaks chosen from the peaks listed in Table 28 below (all peaks that can be or have been chosen from Table 28 are rounded to the nearest 0.1° 2θ). In some embodiments, the crystalline Form 1 Compound 1 fumarate or hydrate or solvate thereof has an X-ray powder diffraction pattern comprising three or more peaks chosen from the peaks listed in Table 28 below (all peaks that can be or have been chosen from Table 28 are rounded to the nearest 0.1° 2θ).

TABLE 28
Peak list of crystalline Form 1 Compound 1 fumarate

| No. | Pos. [° 2θ] | d-spacing [Å] | Height [cts] | Rel. Int. [%] |
|---|---|---|---|---|
| 1 | 8.4241 | 10.49633 | 1248.59 | 42.17 |
| 2 | 9.5776 | 9.23467 | 2247.03 | 75.89 |
| 3 | 10.3419 | 8.55386 | 1813.23 | 61.24 |
| 4 | 10.5986 | 8.34726 | 1007.07 | 34.01 |
| 5 | 11.7407 | 7.53766 | 218.45 | 7.38 |
| 6 | 14.5205 | 6.10032 | 562.42 | 18.99 |
| 7 | 14.9081 | 5.94259 | 341.82 | 11.54 |
| 8 | 16.7003 | 5.30867 | 672.9 | 22.72 |
| 9 | 16.9256 | 5.23851 | 782.43 | 26.42 |
| 10 | 17.1752 | 5.16295 | 808 | 27.29 |
| 11 | 17.6812 | 5.0163 | 586.24 | 19.8 |
| 12 | 18.4509 | 4.80875 | 759.41 | 25.65 |
| 13 | 18.7781 | 4.7257 | 1818.35 | 61.41 |
| 14 | 19.2387 | 4.61359 | 416.6 | 14.07 |
| 15 | 20.7801 | 4.27471 | 1127.21 | 38.07 |
| 16 | 21.9328 | 4.0526 | 537.79 | 18.16 |
| 17 | 22.6434 | 3.92699 | 418.54 | 14.13 |
| 18 | 23.081 | 3.85351 | 317.58 | 10.73 |
| 19 | 23.4126 | 3.79968 | 642.29 | 21.69 |
| 20 | 24.1812 | 3.68062 | 447.39 | 15.11 |
| 21 | 25.1625 | 3.53927 | 2961.08 | 100 |
| 22 | 25.6854 | 3.46839 | 698.77 | 23.6 |
| 23 | 26.3044 | 3.38817 | 749.71 | 25.32 |
| 24 | 26.7534 | 3.33231 | 361.08 | 12.19 |
| 25 | 27.1254 | 3.28745 | 734.98 | 24.82 |
| 26 | 28.4017 | 3.14255 | 132.13 | 4.46 |
| 27 | 29.0565 | 3.0732 | 164.78 | 5.56 |
| 28 | 30.4686 | 2.93392 | 63.91 | 2.16 |
| 29 | 31.2905 | 2.85871 | 33.43 | 1.13 |
| 30 | 32.4756 | 2.75705 | 167.74 | 5.66 |
| 31 | 33.4432 | 2.67946 | 51.73 | 1.75 |
| 32 | 33.8036 | 2.6517 | 81.32 | 2.75 |

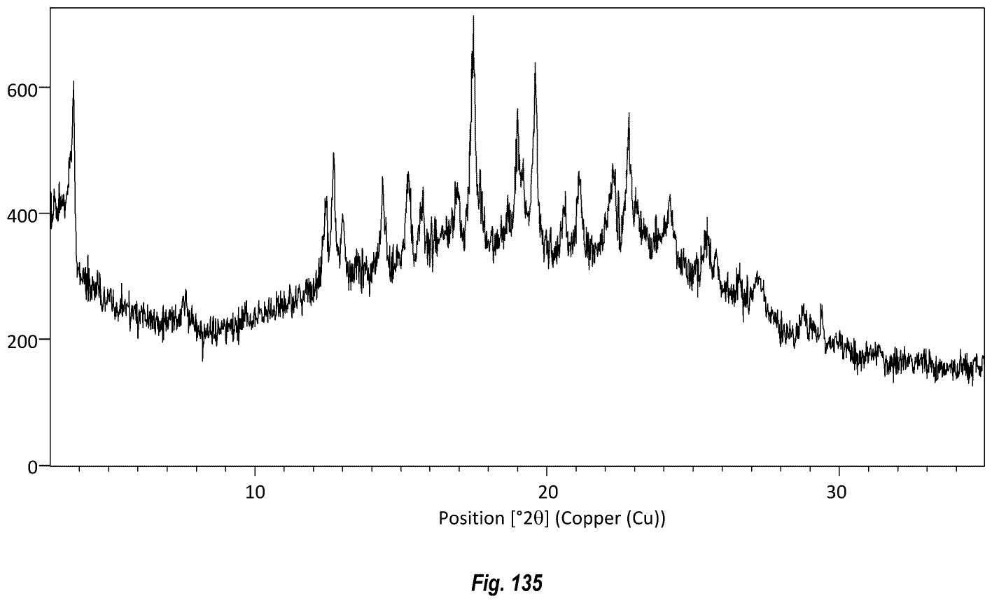

In some embodiments, the present disclosure relates to crystalline Compound 1 fumarate and hydrates and solvates thereof and crystalline Compound 1 mono(fumarate) and hydrates and solvates thereof, including crystalline Form 2 Compound 1 fumarate or a hydrate or solvate thereof, having an X-ray powder diffraction pattern comprising a peak at about 3.8° 2θ. In some embodiments, the crystalline Compound 1 fumarate or hydrate or solvate thereof or the crystalline Compound 1 mono(fumarate) or hydrate or solvate thereof, including crystalline Form 2 Compound 1 fumarate or a hydrate or solvate thereof, has an X-ray powder diffraction pattern comprising peaks at about 3.8° 2θ and about 12.7° 2θ. In some embodiments, the crystalline Compound 1 fumarate or hydrate or solvate thereof or the crystalline Compound 1 mono(fumarate) or hydrate or solvate thereof, including crystalline Form 2 Compound 1 fumarate or a hydrate or solvate thereof, has an X-ray powder diffraction pattern substantially the same as that of FIG. 135.

In some embodiments, the present disclosure relates to crystalline Form 2 Compound 1 fumarate and hydrates and solvates thereof. In some embodiments, the crystalline Form 2 Compound 1 fumarate or hydrate or solvate thereof is crystalline Form 2 Compound 1 mono(fumarate) or hydrate or solvate thereof.

In some embodiments, the crystalline Form 2 Compound 1 fumarate or hydrate or solvate thereof has an X-ray powder diffraction pattern comprising one or more peaks chosen from the peaks listed in Table 29 below (all peaks that can be or have been chosen from Table 29 are rounded to the nearest 0.1° 2θ). In some embodiments, the crystalline Form 2 Compound 1 fumarate or hydrate or solvate thereof has an X-ray powder diffraction pattern comprising two or more peaks chosen from the peaks listed in Table 29 below (all peaks that can be or have been chosen from Table 29 are rounded to the nearest 0.1° 2θ). In some embodiments, the crystalline Form 2 Compound 1 fumarate or hydrate or solvate thereof has an X-ray powder diffraction pattern comprising three or more peaks chosen from the peaks listed in Table 29 below (all peaks that can be or have been chosen from Table 29 are rounded to the nearest 0.1° 2θ).

TABLE 29
Peak list of crystalline Form 2 Compound 1 fumarate

| No. | Pos. [° 2θ] | d-spacing [Å] | Height [cts] | Rel. Int. [%] |
|---|---|---|---|---|
| 1 | 3.8046 | 23.22401 | 364.81 | 81.68 |
| 2 | 7.5546 | 11.70238 | 28.58 | 6.4 |
| 3 | 12.6999 | 6.97043 | 245.99 | 55.08 |
| 4 | 14.38 | 6.15959 | 204.49 | 45.79 |
| 5 | 15.234 | 5.81619 | 219.68 | 49.18 |
| 6 | 15.7015 | 5.64404 | 169.17 | 37.88 |
| 7 | 16.9339 | 5.23596 | 208.46 | 46.67 |
| 8 | 17.4795 | 5.07373 | 446.64 | 100 |
| 9 | 18.9953 | 4.67215 | 327.77 | 73.39 |
| 10 | 19.6109 | 4.52685 | 393.82 | 88.17 |
| 11 | 20.5786 | 4.31612 | 193.62 | 43.35 |
| 12 | 21.11 | 4.20864 | 239.44 | 53.61 |
| 13 | 22.31 | 3.98492 | 247.57 | 55.43 |
| 14 | 22.7772 | 3.90422 | 318.05 | 71.21 |
| 15 | 24.221 | 3.67467 | 212.77 | 47.64 |
| 16 | 25.4561 | 3.49911 | 169.32 | 37.91 |
| 17 | 27.2465 | 3.2731 | 106.54 | 23.85 |
| 18 | 28.9861 | 3.08052 | 35.51 | 7.95 |

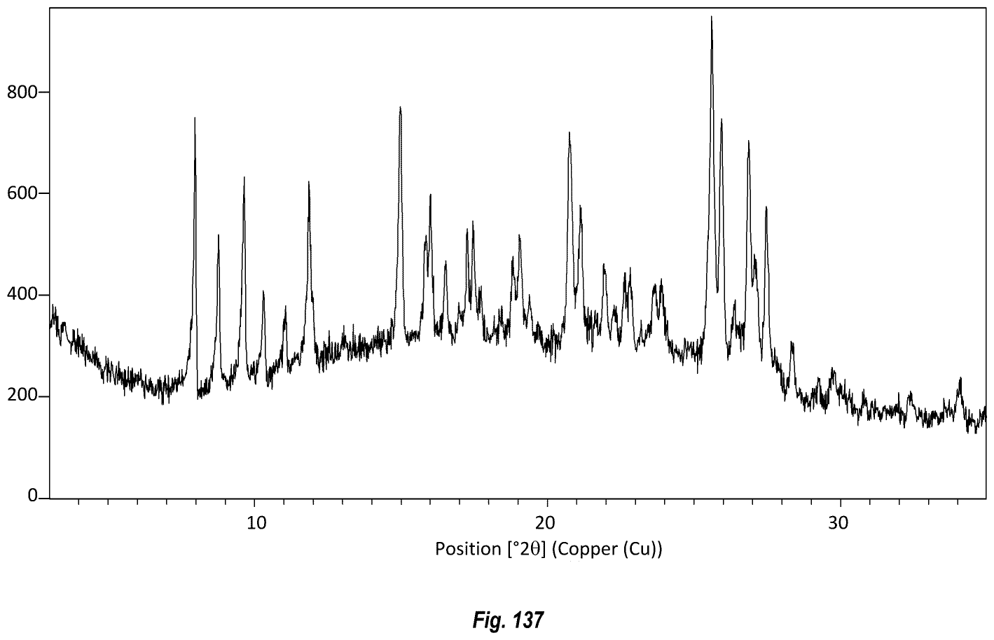

In some embodiments, the present disclosure relates to crystalline Compound 1 fumarate and hydrates and solvates thereof and crystalline Compound 1 mono(fumarate) and hydrates and solvates thereof, including crystalline Form 3 Compound 1 fumarate or a hydrate or solvate thereof, having an X-ray powder diffraction pattern comprising a peak at about 8.8° 2θ. In some embodiments, the crystalline Compound 1 fumarate or hydrate or solvate thereof or the crystalline Compound 1 mono(fumarate) or hydrate or solvate thereof, including crystalline Form 3 Compound 1 fumarate or a hydrate or solvate thereof, has an X-ray powder diffraction pattern comprising peaks at about 8.8° 2θ and about 15.8° 2θ. In some embodiments, the crystalline Compound 1 fumarate or hydrate or solvate thereof or the crystalline Compound 1 mono(fumarate) or hydrate or solvate thereof, including crystalline Form 3 Compound 1 fumarate or a hydrate or solvate thereof, has an X-ray powder diffraction pattern substantially the same as that of FIG. 137.

In some embodiments, the present disclosure relates to crystalline Form 3 Compound 1 fumarate and hydrates and solvates thereof. In some embodiments, the crystalline Form 3 Compound 1 fumarate or hydrate or solvate thereof is crystalline Form 3 Compound 1 mono(fumarate) or hydrate or solvate thereof.

In some embodiments, the crystalline Form 3 Compound 1 fumarate or hydrate or solvate thereof has an X-ray powder diffraction pattern comprising one or more peaks chosen from the peaks listed in Table 30 below (all peaks that can be or have been chosen from Table 30 are rounded to the nearest 0.1° 2θ). In some embodiments, the crystalline Form 3 Compound 1 fumarate or hydrate or solvate thereof has an X-ray powder diffraction pattern comprising two or more peaks chosen from the peaks listed in Table 30 below (all peaks that can be or have been chosen from Table 30 are rounded to the nearest 0.1° 2θ). In some embodiments, the crystalline Form 3 Compound 1 fumarate or hydrate or solvate thereof has an X-ray powder diffraction pattern comprising three or more peaks chosen from the peaks listed in Table 30 below (all peaks that can be or have been chosen from Table 30 are rounded to the nearest 0.1° 2θ).

TABLE 30
Peak list of crystalline Form 3 Compound 1 fumarate

| No. | Pos. [° 2θ] | d-spacing [Å] | Height [cts] | Rel. Int. [%] |
|---|---|---|---|---|
| 1 | 7.9774 | 11.08309 | 520.44 | 69.01 |
| 2 | 8.7732 | 10.07948 | 288.57 | 38.26 |
| 3 | 9.6541 | 9.16168 | 391.66 | 51.93 |
| 4 | 10.3057 | 8.58377 | 162.38 | 21.53 |
| 5 | 11.0487 | 8.00817 | 89.67 | 11.89 |
| 6 | 11.8678 | 7.45725 | 348.1 | 46.16 |
| 7 | 14.9836 | 5.91281 | 496.19 | 65.79 |
| 8 | 15.8295 | 5.59867 | 231.2 | 30.66 |
| 9 | 16.0116 | 5.53541 | 305.19 | 40.47 |
| 10 | 16.5104 | 5.36929 | 177.56 | 23.54 |
| 11 | 17.2546 | 5.13937 | 260.39 | 34.53 |
| 12 | 17.4606 | 5.07919 | 285.09 | 37.8 |
| 13 | 18.7999 | 4.72027 | 195.62 | 25.94 |
| 14 | 19.0424 | 4.66068 | 259.84 | 34.45 |
| 15 | 20.7479 | 4.28127 | 476.44 | 63.17 |
| 16 | 21.1432 | 4.20211 | 330.61 | 43.84 |
| 17 | 21.9468 | 4.05004 | 224.97 | 29.83 |
| 18 | 22.6264 | 3.92991 | 151.67 | 20.11 |
| 19 | 22.8257 | 3.89605 | 210.89 | 27.96 |
| 20 | 23.6326 | 3.76481 | 194.15 | 25.74 |
| 21 | 25.6004 | 3.47972 | 754.19 | 100 |
| 22 | 25.9491 | 3.43374 | 530.07 | 70.28 |
| 23 | 26.3589 | 3.38128 | 167.96 | 22.27 |
| 24 | 26.8852 | 3.31627 | 498.06 | 66.04 |
| 25 | 27.0911 | 3.29154 | 261.78 | 34.71 |
| 26 | 27.451 | 3.2492 | 373.06 | 49.46 |
| 27 | 28.3491 | 3.14826 | 105.78 | 14.03 |
| 28 | 29.7389 | 3.00423 | 63.92 | 8.48 |
| 29 | 32.3752 | 2.76537 | 37.46 | 4.97 |
| 30 | 34.0646 | 2.63199 | 67.37 | 8.93 |

In one aspect, the present disclosure relates to Compound 1 L-malate and hydrates and solvates thereof. Compound 1 L-malate is formed by, for example, the combination of Compound 1 (free base) and L-malic acid and can be represented as shown below where n is a number greater than zero.

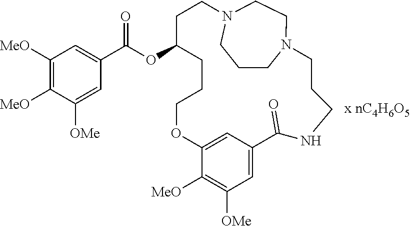 [SMILES: COc1cc(C(=O)O[C@@H]2CCCOc3cc(cc(OC)c3OC)C(=O)NCCCN3CCCN(CC2)CC3)cc(OC)c1OC ; "x nC4H6O5"]

In some embodiments, the present disclosure relates to Compound 1 mono(L-malate) and hydrates and solvates thereof. Compound 1 mono(L-malate) is formed by, for example, the combination of Compound 1 (free base) and one molecular equivalent of L-malic acid and can be represented as shown below.

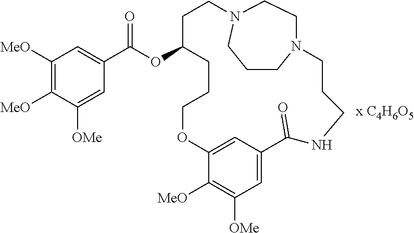 [SMILES: COc1cc(C(=O)O[C@@H]2CCCOc3cc(cc(OC)c3OC)C(=O)NCCCN3CCCN(CC2)CC3)cc(OC)c1OC ; "x C4H6O5"]

In some embodiments, the present disclosure relates to Compound 1 di(L-malate) and hydrates and solvates thereof. Compound 1 mono(L-malate) is formed by, for example, the combination of Compound 1 (free base) and two molecular equivalents of L-malic acid and can be represented as shown below.

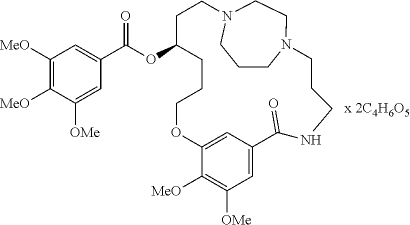 [SMILES: COc1cc(C(=O)O[C@@H]2CCCOc3cc(cc(OC)c3OC)C(=O)NCCCN3CCCN(CC2)CC3)cc(OC)c1OC ; "x 2C4H6O5"]

In some embodiments, the present disclosure relates to amorphous Compound 1 L-malate and hydrates and solvates thereof. In some embodiments, the present disclosure relates to amorphous Compound 1 mono(L-malate) and hydrates and solvates thereof. In some embodiments, the present disclosure relates to amorphous Compound 1 di(L-malate) and hydrates and solvates thereof.

In some embodiments, the present disclosure relates to crystalline Compound 1 L-malate and hydrates and solvates thereof. In some embodiments, the present disclosure relates to crystalline Compound 1 mono(L-malate) and hydrates and solvates thereof.

In some embodiments, the present disclosure relates to crystalline Compound 1 di(L-malate) and hydrates and solvates thereof.

In some embodiments, the crystalline Compound 1 L-malate or hydrate or solvate thereof or the crystalline Compound 1 mono(L-malate) or hydrate or solvate thereof has an X-ray powder diffraction pattern comprising a peak at about 4.3° 2θ.

In some embodiments, the crystalline Compound 1 L-malate or hydrate or solvate thereof or the crystalline Compound 1 mono(L-malate) or hydrate or solvate thereof has an X-ray powder diffraction pattern comprising one or more peaks at about 4.3° 2θ, about 10.1° 2θ, about 11.4° 2θ, and about 12.8° 2θ.

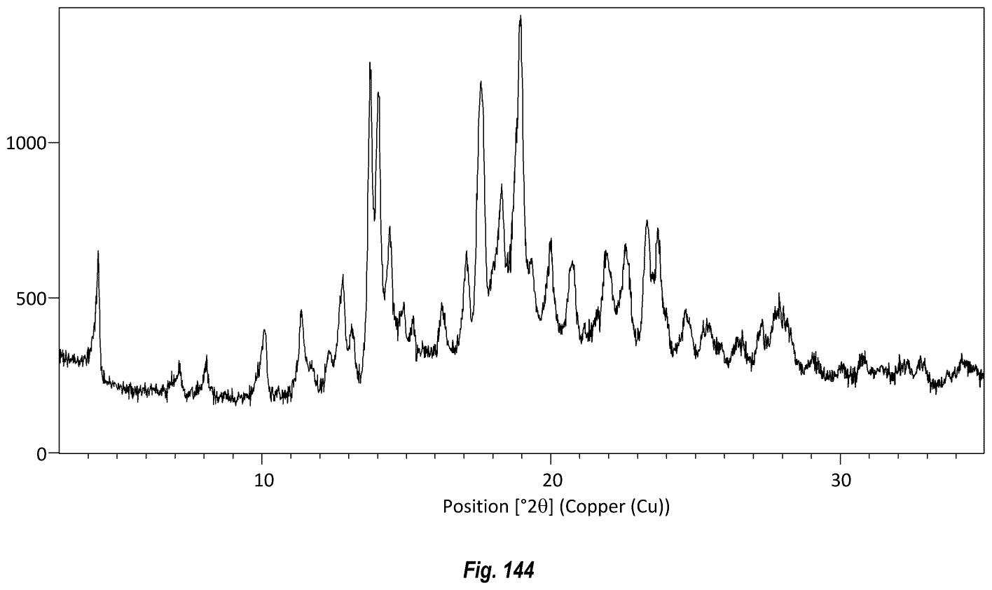

In some embodiments, the crystalline Compound 1 L-malate or hydrate or solvate thereof or the crystalline Compound 1 mono(L-malate) or hydrate or solvate thereof has an X-ray powder diffraction pattern substantially the same as that of FIG. 144.

In some embodiments, the crystalline Compound 1 L-malate or hydrate or solvate thereof has an X-ray powder diffraction pattern comprising one or more peaks chosen from the peaks listed in Table 31 below (all peaks that can be or have been chosen from Table 31 are rounded to the nearest 0.1° 2θ). In some embodiments, the crystalline Compound 1 L-malate or hydrate or solvate thereof has an X-ray powder diffraction pattern comprising two or more peaks chosen from the peaks listed in Table 31 below (all peaks that can be or have been chosen from Table 31 are rounded to the nearest 0.1° 2θ). In some embodiments, the crystalline Compound 1 L-malate or hydrate or solvate thereof has an X-ray powder diffraction pattern comprising three or more peaks chosen from the peaks listed in Table 31 below (all peaks that can be or have been chosen from Table 31 are rounded to the nearest 0.1° 2θ).

TABLE 31
Peak list of crystalline Compound 1 L-malate

| No. | Pos. [° 2θ] | d-spacing [Å] | Height [cts] | Rel. Int. [%] |
|---|---|---|---|---|
| 1 | 4.3457 | 20.33387 | 413.12 | 33.54 |
| 2 | 7.1611 | 12.34453 | 88.17 | 7.16 |
| 3 | 8.0829 | 10.93864 | 112.69 | 9.15 |
| 4 | 10.1115 | 8.74819 | 219.59 | 17.83 |
| 5 | 11.3648 | 7.78615 | 263.07 | 21.36 |
| 6 | 12.3397 | 7.17308 | 144.97 | 11.77 |
| 7 | 12.7997 | 6.91633 | 390.73 | 31.73 |
| 8 | 13.1439 | 6.73594 | 202.06 | 16.41 |
| 9 | 13.7502 | 6.44031 | 1089.83 | 88.49 |
| 10 | 14.0428 | 6.30677 | 983.24 | 79.84 |
| 11 | 14.4464 | 6.13143 | 517.95 | 42.06 |
| 12 | 14.8778 | 5.9546 | 280.69 | 22.79 |
| 13 | 15.2547 | 5.80832 | 231.58 | 18.8 |
| 14 | 16.2405 | 5.45791 | 281.9 | 22.89 |
| 15 | 17.0591 | 5.19781 | 442.73 | 35.95 |
| 16 | 17.6249 | 5.03222 | 969.58 | 78.73 |
| 17 | 18.3043 | 4.84693 | 670.72 | 54.46 |
| 18 | 18.9491 | 4.68342 | 1231.54 | 100 |
| 19 | 19.3528 | 4.58663 | 421.09 | 34.19 |
| 20 | 20.0003 | 4.43958 | 487.04 | 39.55 |
| 21 | 20.7919 | 4.2723 | 404.04 | 32.81 |
| 22 | 21.8866 | 4.06105 | 433.68 | 35.21 |
| 23 | 22.5748 | 3.93877 | 469 | 38.08 |
| 24 | 23.3544 | 3.80901 | 537.8 | 43.67 |
| 25 | 23.6894 | 3.75592 | 510.78 | 41.47 |
| 26 | 24.6799 | 3.60737 | 247.23 | 20.08 |
| 27 | 25.3859 | 3.50863 | 200.98 | 16.32 |
| 28 | 26.5174 | 3.36142 | 149.62 | 12.15 |
| 29 | 27.243 | 3.27352 | 187.61 | 15.23 |
| 30 | 27.8261 | 3.20624 | 240.94 | 19.56 |
| 31 | 29.088 | 3.06996 | 82.14 | 6.67 |
| 32 | 30.7495 | 2.90775 | 88.87 | 7.22 |
| 33 | 32.8596 | 2.7257 | 57.03 | 4.63 |

In one aspect, the present disclosure relates to Compound 1 benzoate and hydrates and solvates thereof. Compound 1 benzoate is formed by, for example, the combination of Compound 1 (free base) and benzoic acid and can be represented as shown below where n is a number greater than zero.

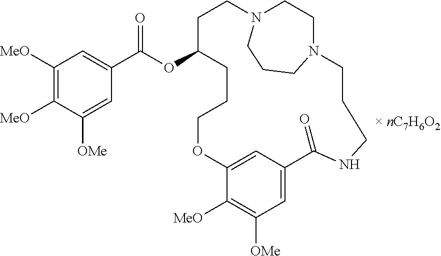 [SMILES: COc1cc(C(=O)O[C@@H]2CCCOc3cc(cc(OC)c3OC)C(=O)NCCCN3CCCN(CC2)CC3)cc(OC)c1OC]

In some embodiments, the present disclosure relates to Compound 1 mono(benzoate) and hydrates and solvates thereof. Compound 1 mono(benzoate) is formed by, for example, the combination of Compound 1 (free base) and one molecular equivalent of benzoic acid and can be represented as shown below.

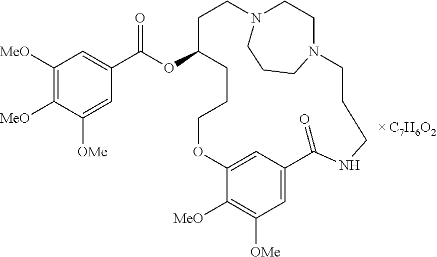 [SMILES: COc1cc(C(=O)O[C@@H]2CCCOc3cc(cc(OC)c3OC)C(=O)NCCCN3CCCN(CC2)CC3)cc(OC)c1OC]

In some embodiments, the present disclosure relates to Compound 1 di(benzoate) and hydrates and solvates thereof. Compound 1 mono(benzoate) is formed by, for example, the combination of Compound 1 (free base) and two molecular equivalents of benzoic acid and can be represented as shown below.

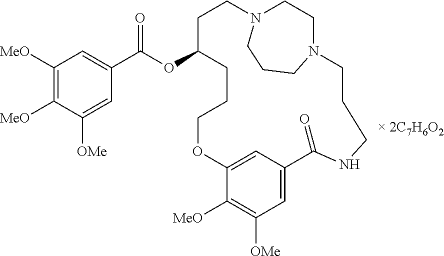 [SMILES: COc1cc(C(=O)O[C@@H]2CCCOc3cc(cc(OC)c3OC)C(=O)NCCCN3CCCN(CC2)CC3)cc(OC)c1OC]

In some embodiments, the present disclosure relates to amorphous Compound 1 benzoate and hydrates and solvates thereof. In some embodiments, the present disclosure relates to amorphous Compound 1 mono(benzoate) and hydrates and solvates thereof. In some embodiments, the present disclosure relates to amorphous Compound 1 di(benzoate) and hydrates and solvates thereof.

In some embodiments, the present disclosure relates to crystalline Compound 1 benzoate and hydrates and solvates thereof. In some embodiments, the present disclosure relates to crystalline Compound 1 mono(benzoate) and hydrates and solvates thereof. In some embodiments, the present disclosure relates to crystalline Compound 1 di(benzoate) and hydrates and solvates thereof.

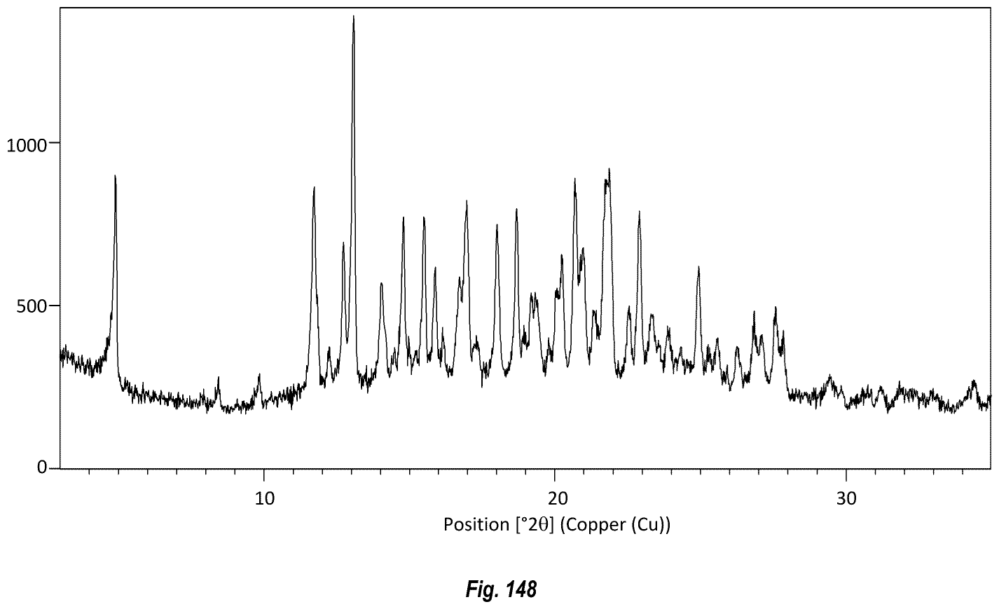

In some embodiments, the present disclosure relates to crystalline Compound 1 benzoate and hydrates and solvates thereof and crystalline Compound 1 mono(benzoate) and hydrates and solvates thereof, including crystalline Form 1 Compound 1 benzoate or a hydrate or solvate thereof, having an X-ray powder diffraction pattern comprising a peak at about 4.9° 2θ. In some embodiments, the crystalline Compound 1 benzoate or hydrate or solvate thereof or the crystalline Compound 1 mono(benzoate) or hydrate or solvate thereof, including crystalline Form 1 Compound 1 benzoate or a hydrate or solvate thereof, has an X-ray powder diffraction pattern comprising peaks at about 4.9° 2θ and about 11.7° 2θ. In some embodiments, the crystalline Compound 1 benzoate or hydrate or solvate thereof or the crystalline Compound 1 mono(benzoate) or hydrate or solvate thereof, including crystalline Form 1 Compound 1 benzoate or a hydrate or solvate thereof, has an X-ray powder diffraction pattern substantially the same as that of FIG. 148.

In some embodiments, the present disclosure relates to crystalline Form 1 Compound 1 benzoate and hydrates and solvates thereof. In some embodiments, the crystalline Form 1 Compound 1 benzoate or hydrate or solvate thereof is crystalline Form 1 Compound 1 mono(benzoate) or hydrate or solvate thereof.

In some embodiments, the crystalline Form 1 Compound 1 benzoate or hydrate or solvate thereof has an X-ray powder diffraction pattern comprising one or more peaks chosen from the peaks listed in Table 32 below (all peaks that can be or have been chosen from Table 32 are rounded to the nearest 0.1° 2θ). In some embodiments, the crystalline Form 1 Compound 1 benzoate or hydrate or solvate thereof has an X-ray powder diffraction pattern comprising two or more peaks chosen from the peaks listed in Table 32 below (all peaks that can be or have been chosen from Table 32 are rounded to the nearest 0.1° 2θ). In some embodiments, the crystalline Form 1 Compound 1 benzoate or hydrate or solvate thereof has an X-ray powder diffraction pattern comprising three or more peaks chosen from the peaks listed in Table 32 below (all peaks that can be or have been chosen from Table 32 are rounded to the nearest 0.1° 2θ).

TABLE 32
Peak list of crystalline Form 1 Compound 1 benzoate

| No. | Pos. [° 2θ] | d-spacing [Å] | Height [cts] | Rel. Int. [%] |
|---|---|---|---|---|
| 1 | 4.9084 | 18.00387 | 633.63 | 52.65 |
| 2 | 8.4195 | 10.50204 | 56.89 | 4.73 |
| 3 | 9.8359 | 8.99268 | 92.72 | 7.7 |
| 4 | 11.7045 | 7.5609 | 654.18 | 54.36 |
| 5 | 12.2398 | 7.23143 | 161.17 | 13.39 |
| 6 | 12.7264 | 6.95597 | 507.06 | 42.14 |
| 7 | 13.0891 | 6.76407 | 1203.4 | 100 |
| 8 | 14.03 | 6.31249 | 381.89 | 31.73 |
| 9 | 14.7901 | 5.9897 | 575.74 | 47.84 |
| 10 | 15.5088 | 5.71374 | 585.58 | 48.66 |
| 11 | 15.8857 | 5.579 | 433.16 | 35.99 |
| 12 | 16.1633 | 5.48382 | 204.05 | 16.96 |
| 13 | 16.7097 | 5.30571 | 389.9 | 32.4 |
| 14 | 16.9742 | 5.22361 | 616.96 | 51.27 |
| 15 | 17.3518 | 5.11078 | 186.34 | 15.48 |
| 16 | 18.0028 | 4.92743 | 559.48 | 46.49 |
| 17 | 18.7069 | 4.74353 | 581.55 | 48.33 |
| 18 | 19.1787 | 4.62787 | 339.14 | 28.18 |
| 19 | 19.3607 | 4.58478 | 313.52 | 26.05 |
| 20 | 20.0353 | 4.43191 | 352.16 | 29.26 |
| 21 | 20.2464 | 4.38617 | 462.13 | 38.4 |
| 22 | 20.6658 | 4.2981 | 616.47 | 51.23 |
| 23 | 20.9881 | 4.23281 | 472.38 | 39.25 |
| 24 | 21.323 | 4.16709 | 281.74 | 23.41 |
| 25 | 21.6966 | 4.09617 | 610.78 | 50.75 |
| 26 | 21.9045 | 4.05777 | 646.03 | 53.68 |
| 27 | 22.5467 | 3.94361 | 283.15 | 23.53 |
| 28 | 22.8841 | 3.88623 | 562.12 | 46.71 |
| 29 | 23.3381 | 3.81164 | 268.21 | 22.29 |
| 30 | 23.8685 | 3.72813 | 219.68 | 18.26 |
| 31 | 24.9481 | 3.56919 | 402.42 | 33.44 |
| 32 | 25.5859 | 3.48165 | 192.84 | 16.02 |
| 33 | 26.2515 | 3.39486 | 167.11 | 13.89 |
| 34 | 26.8314 | 3.32279 | 249.2 | 20.71 |
| 35 | 27.1269 | 3.28727 | 196.46 | 16.33 |
| 36 | 27.5575 | 3.23687 | 260.57 | 21.65 |
| 37 | 27.8412 | 3.20454 | 190.7 | 15.85 |
| 38 | 29.4286 | 3.03519 | 62.94 | 5.23 |
| 39 | 31.1998 | 2.86681 | 33.39 | 2.77 |
| 40 | 34.4103 | 2.60633 | 74.16 | 6.16 |

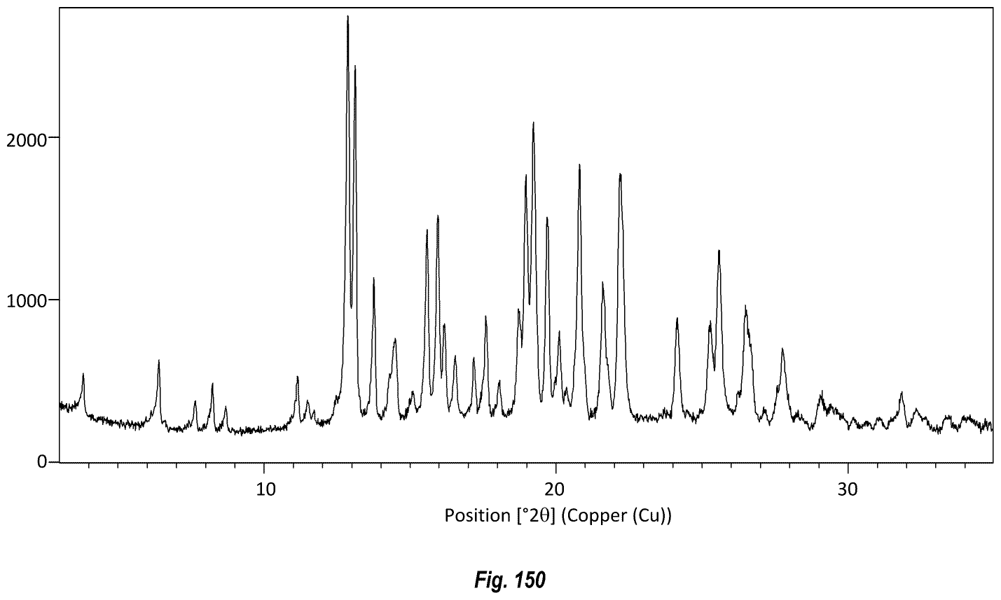

In some embodiments, the present disclosure relates to crystalline Compound 1 benzoate and hydrates and solvates thereof and crystalline Compound 1 mono(benzoate) and hydrates and solvates thereof, including crystalline Form 2 Compound 1 benzoate or a hydrate or solvate thereof, having an X-ray powder diffraction pattern comprising a peak at about 3.8° 2θ. In some embodiments, the crystalline Compound 1 benzoate or hydrate or solvate thereof or the crystalline Compound 1 mono(benzoate) or hydrate or solvate thereof, including crystalline Form 2 Compound 1 benzoate or a hydrate or solvate thereof, has an X-ray powder diffraction pattern substantially the same as that of FIG. 150.

In some embodiments, the present disclosure relates to crystalline Form 2 Compound 1 benzoate and hydrates and solvates thereof. In some embodiments, the crystalline Form 2 Compound 1 benzoate or hydrate or solvate thereof is crystalline Form 2 Compound 1 mono(benzoate) or hydrate or solvate thereof.

In some embodiments, the crystalline Form 2 Compound 1 benzoate or hydrate or solvate thereof has an X-ray powder diffraction pattern comprising one or more peaks chosen from the peaks listed in Table 33 below (all peaks that can be or have been chosen from Table 33 are rounded to the nearest 0.1° 2θ). In some embodiments, the crystalline Form 2 Compound 1 benzoate or hydrate or solvate thereof has an X-ray powder diffraction pattern comprising two or more peaks chosen from the peaks listed in Table 33 below (all peaks that can be or have been chosen from Table 33 are rounded to the nearest 0.1° 2θ). In some embodiments, the crystalline Form 2 Compound 1 benzoate or hydrate or solvate thereof has an X-ray powder diffraction pattern comprising three or more peaks chosen from the peaks listed in Table 33 below (all peaks that can be or have been chosen from Table 33 are rounded to the nearest 0.1° 2θ).

TABLE 33
Peak list of crystalline Form 2 Compound 1 benzoate

| No. | Pos. [° 2θ] | d-spacing [Å] | Height [cts] | Rel. Int. [%] |
|---|---|---|---|---|
| 1 | 3.8147 | 23.16294 | 362.7 | 14.09 |
| 2 | 6.4086 | 13.79224 | 451.18 | 17.52 |
| 3 | 7.6526 | 11.55274 | 189.33 | 7.35 |
| 4 | 8.2446 | 10.7245 | 308.35 | 11.97 |
| 5 | 8.685 | 10.1816 | 149.33 | 5.8 |
| 6 | 11.1591 | 7.92921 | 338.09 | 13.13 |
| 7 | 11.5039 | 7.69232 | 187.18 | 7.27 |
| 8 | 12.8823 | 6.87218 | 2575.02 | 100 |
| 9 | 13.1228 | 6.74677 | 2284.1 | 88.7 |
| 10 | 13.7654 | 6.43319 | 924.69 | 35.91 |
| 11 | 14.522 | 6.09969 | 522.5 | 20.29 |
| 12 | 15.1012 | 5.86701 | 242.16 | 9.4 |
| 13 | 15.586 | 5.6856 | 1224.71 | 47.56 |
| 14 | 15.9649 | 5.55153 | 1345.9 | 52.27 |
| 15 | 16.188 | 5.4755 | 668.06 | 25.94 |
| 16 | 16.5373 | 5.36061 | 470.71 | 18.28 |
| 17 | 17.1685 | 5.16495 | 451.08 | 17.52 |
| 18 | 17.6077 | 5.0371 | 672.26 | 26.11 |
| 19 | 18.0516 | 4.91421 | 289.14 | 11.23 |
| 20 | 18.727 | 4.73847 | 739.85 | 28.73 |
| 21 | 18.9659 | 4.67931 | 1608.34 | 62.46 |
| 22 | 19.2145 | 4.61934 | 1930.27 | 74.96 |
| 23 | 19.6741 | 4.51244 | 1265.82 | 49.16 |
| 24 | 20.1054 | 4.4166 | 623.64 | 24.22 |
| 25 | 20.8075 | 4.26914 | 1665.98 | 64.7 |
| 26 | 21.579 | 4.11823 | 805.42 | 31.28 |
| 27 | 22.1627 | 4.01106 | 1536.51 | 59.67 |
| 28 | 24.1541 | 3.68469 | 692.3 | 26.89 |
| 29 | 25.2648 | 3.52516 | 637.48 | 24.76 |
| 30 | 25.5702 | 3.48376 | 1121.83 | 43.57 |
| 31 | 26.4809 | 3.36598 | 741.55 | 28.8 |
| 32 | 26.7068 | 3.33802 | 446.3 | 17.33 |
| 33 | 27.7662 | 3.21301 | 494.67 | 19.21 |
| 34 | 29.0796 | 3.07082 | 202.23 | 7.85 |
| 35 | 31.0574 | 2.87963 | 59.14 | 2.3 |
| 36 | 31.818 | 2.81251 | 212.55 | 8.25 |
| 37 | 32.3302 | 2.76911 | 114.1 | 4.43 |
| 38 | 33.3911 | 2.68352 | 58.84 | 2.28 |

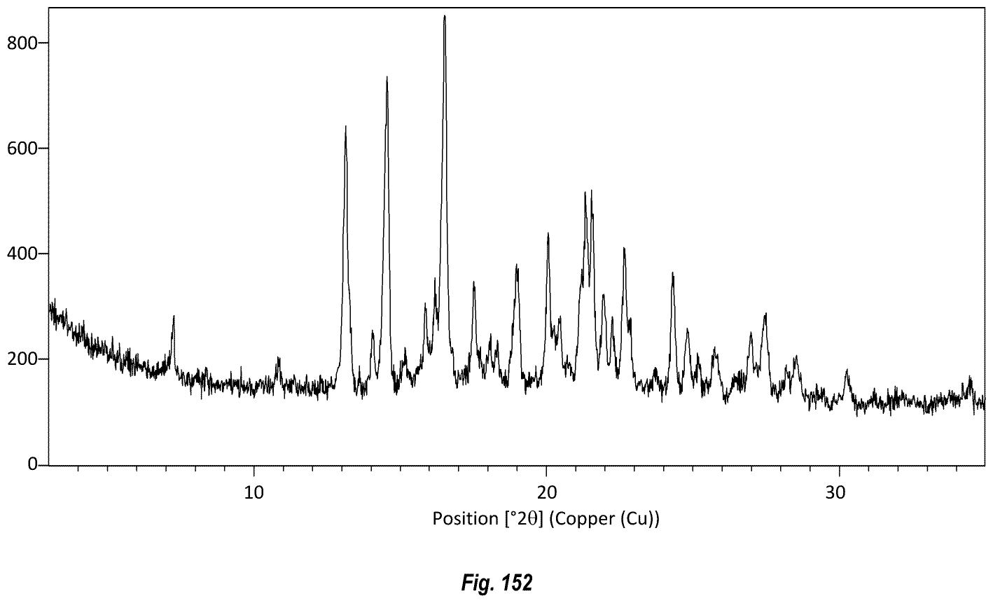

In some embodiments, the present disclosure relates to crystalline Compound 1 benzoate and hydrates and solvates thereof and crystalline Compound 1 mono(benzoate) and hydrates and solvates thereof, including crystalline Form 3 Compound 1 benzoate or a hydrate or solvate thereof, having an X-ray powder diffraction pattern comprising a peak at about 7.2° 2θ. In some embodiments, the crystalline Compound 1 benzoate or hydrate or solvate thereof or the crystalline Compound 1 mono(benzoate) or hydrate or solvate thereof, including crystalline Form 3 Compound 1 benzoate or a hydrate or solvate thereof, has an X-ray powder diffraction pattern substantially the same as that of FIG. 152.

In some embodiments, the present disclosure relates to crystalline Form 3 Compound 1 benzoate and hydrates and solvates thereof. In some embodiments, the crystalline Form 3 Compound 1 benzoate or hydrate or solvate thereof is crystalline Form 3 Compound 1 mono(benzoate) or hydrate or solvate thereof.

In some embodiments, the crystalline Form 3 Compound 1 benzoate or hydrate or solvate thereof has an X-ray powder diffraction pattern comprising one or more peaks chosen from the peaks listed in Table 34 below (all peaks that can be or have been chosen from Table 34 are rounded to the nearest 0.1° 2θ). In some embodiments, the crystalline Form 3 Compound 1 benzoate or hydrate or solvate thereof has an X-ray powder diffraction pattern comprising two or more peaks chosen from the peaks listed in Table 34 below (all peaks that can be or have been chosen from Table 34 are rounded to the nearest 0.1° 2θ). In some embodiments, the crystalline Form 3 Compound 1 benzoate or hydrate or solvate thereof has an X-ray powder diffraction pattern comprising three or more peaks chosen from the peaks listed in Table 34 below (all peaks that can be or have been chosen from Table 34 are rounded to the nearest 0.1° 2θ).

TABLE 34
Peak list of crystalline Form 3 Compound 1 benzoate

| No. | Pos. [° 2θ] | d-spacing [Å] | Height [cts] | Rel. Int. [%] |
|---|---|---|---|---|
| 1 | 7.2427 | 12.20562 | 106.16 | 15.4 |
| 2 | 10.8366 | 8.16442 | 48.02 | 6.97 |
| 3 | 13.15 | 6.73285 | 483.84 | 70.2 |
| 4 | 14.0578 | 6.30006 | 110.02 | 15.96 |
| 5 | 14.5643 | 6.08207 | 579.92 | 84.14 |
| 6 | 15.132 | 5.85517 | 61.88 | 8.98 |
| 7 | 15.8634 | 5.5868 | 167.69 | 24.33 |
| 8 | 16.1831 | 5.47713 | 203.04 | 29.46 |
| 9 | 16.544 | 5.35847 | 689.23 | 100 |
| 10 | 17.5168 | 5.06303 | 215.87 | 31.32 |
| 11 | 18.9954 | 4.67211 | 240.4 | 34.88 |
| 12 | 20.0438 | 4.43004 | 306.89 | 44.53 |
| 13 | 20.4553 | 4.34185 | 145.71 | 21.14 |
| 14 | 21.3371 | 4.16436 | 385.06 | 55.87 |
| 15 | 21.5616 | 4.12151 | 354.83 | 51.48 |
| 16 | 21.9533 | 4.04885 | 194.33 | 28.19 |
| 17 | 22.2331 | 3.99853 | 145.3 | 21.08 |
| 18 | 22.6626 | 3.92371 | 285.45 | 41.42 |
| 19 | 24.3134 | 3.66091 | 236.39 | 34.3 |
| 20 | 24.834 | 3.58534 | 129.39 | 18.77 |
| 21 | 25.1809 | 3.53672 | 69.96 | 10.15 |
| 22 | 25.748 | 3.4601 | 90.82 | 13.18 |
| 23 | 26.9847 | 3.30427 | 126.11 | 18.3 |
| 24 | 27.4599 | 3.24816 | 163.71 | 23.75 |
| 25 | 28.5324 | 3.12846 | 82.62 | 11.99 |
| 26 | 30.263 | 2.95338 | 59.74 | 8.67 |

In one aspect, the present disclosure relates to Compound 1 tosylate and hydrates and solvates thereof. Compound 1 tosylate is formed by, for example, the combination of Compound 1 (free base) and p-toluenesulfonic acid and can be represented as shown below where n is a number greater than zero.

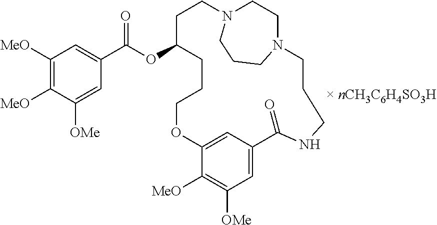 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 mono(tosylate) and hydrates and solvates thereof. Compound 1 mono(tosylate) is formed by, for example, the combination of Compound 1 (free base) and one molecular equivalent of p-toluenesulfonic acid and can be represented as shown below.

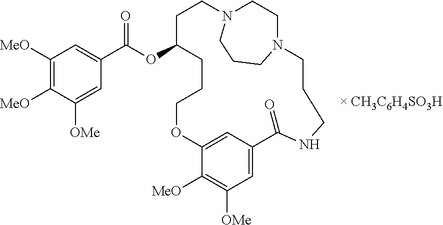 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 di(tosylate) and hydrates and solvates thereof. Compound 1 di(tosylate) is formed by, for example, the combination of Compound 1 (free base) and two molecular equivalents of p-toluenesulfonic acid and can be represented as shown below.

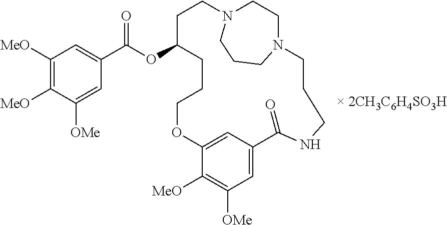 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to amorphous Compound 1 tosylate and hydrates and solvates thereof. In one aspect, the present disclosure relates to amorphous Compound 1 mono(tosylate) and hydrates and solvates thereof. In one aspect, the present disclosure relates to amorphous Compound 1 di(tosylate) and hydrates and solvates thereof.

In one aspect, the present disclosure relates to Compound 1 mesylate and hydrates and solvates thereof. Compound 1 mesylate is formed by, for example, the combination of Compound 1 (free base) and methanesulfonic acid and can be represented as shown below where n is a number greater than zero.

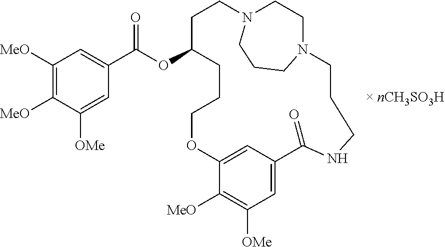 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 mono(mesylate) and hydrates and solvates thereof. Compound 1 mono(mesylate) is formed by, for example, the combination of Compound 1 (free base) and one molecular equivalent of methanesulfonic acid and can be represented as shown below.

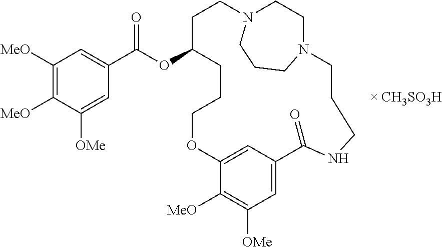 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 di(mesylate) and hydrates and solvates thereof. Compound 1 di(mesylate) is formed by, for example, the combination of Compound 1 (free base) and two molecular equivalents of methanesulfonic acid and can be represented as shown below.

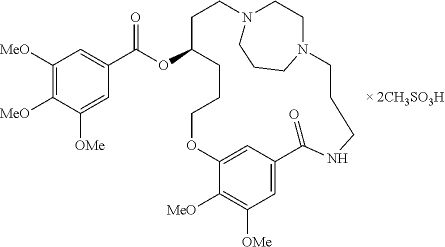 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to amorphous Compound 1 mesylate and hydrates and solvates thereof. In one aspect, the present disclosure relates to amorphous Compound 1 mono(mesylate) and hydrates and solvates thereof. In one aspect, the present disclosure relates to amorphous Compound 1 di(mesylate) and hydrates and solvates thereof.

In one aspect, the present disclosure relates to Compound 1 benzenesulfonate and hydrates and solvates thereof. Compound 1 benzenesulfonate is formed by, for example, the combination of Compound 1 (free base) and benzenesulfonic acid and can be represented as shown below where n is a number greater than zero.

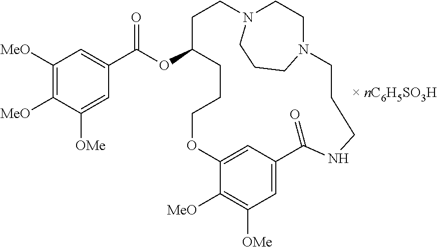 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 mono(benzenesulfonate) and hydrates and solvates thereof. Compound 1 mono(benzenesulfonate) is formed by, for example, the combination of Compound 1 (free base) and one molecular equivalent of benzenesulfonic acid and can be represented as shown below.

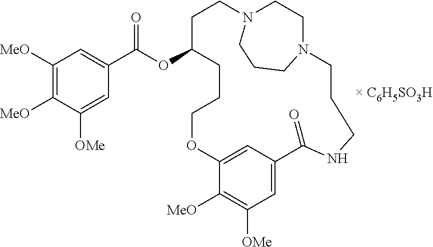 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 di(benzenesulfonate) and hydrates and solvates thereof. Compound 1 di(benzenesulfonate) is formed by, for example, the combination of Compound 1 (free base) and two molecular equivalents of benzenesulfonic acid and can be represented as shown below.

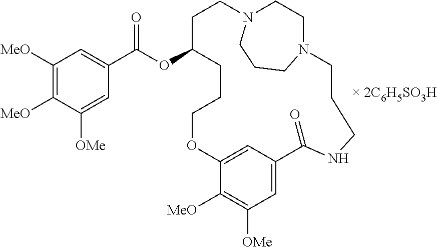 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to amorphous Compound 1 benzenesulfonate and hydrates and solvates thereof. In one aspect, the present disclosure relates to amorphous Compound 1 mono(benzenesulfonate) and hydrates and solvates thereof. In one aspect, the present disclosure relates to amorphous Compound 1 di(benzenesulfonate) and hydrates and solvates thereof.

In one aspect, the present disclosure relates to Compound 1 L-tartrate and hydrates and solvates thereof. Compound 1 L-tartrate is formed by, for example, the combination of Compound 1 (free base) and L-tartaric acid and can be represented as shown below where n is a number greater than zero.

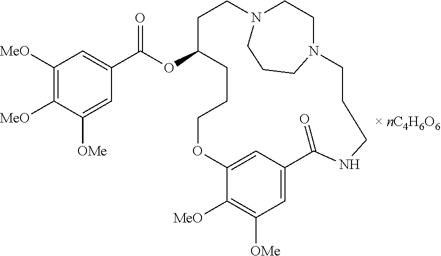 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 mono(L-tartrate) and hydrates and solvates thereof. Compound 1 mono(L-tartrate) is formed by, for example, the combination of Compound 1 (free base) and one molecular equivalent of L-tartaric acid and can be represented as shown below.

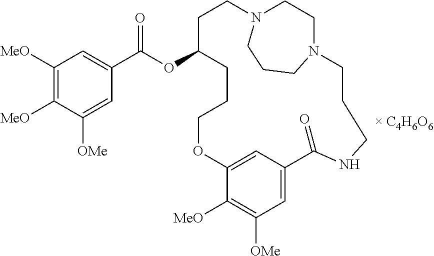 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 di(L-tartrate) and hydrates and solvates thereof. Compound 1 di(L-tartrate) is formed by, for example, the combination of Compound 1 (free base) and two molecular equivalents of L-tartaric acid and can be represented as shown below.

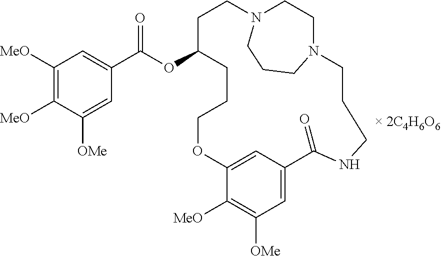 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to amorphous Compound 1 L-tartrate and hydrates and solvates thereof. In one aspect, the present disclosure relates to amorphous Compound 1 mono(L-tartrate) and hydrates and solvates thereof. In one aspect, the present disclosure relates to amorphous Compound 1 di(L-tartrate) and hydrates and solvates thereof.

In one aspect, the present disclosure relates to Compound 1 citrate and hydrates and solvates thereof. Compound 1 citrate is formed by, for example, the combination of Compound 1 (free base) and citric acid and can be represented as shown below where n is a number greater than zero.

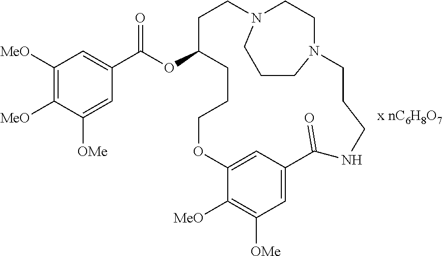 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 mono(citrate) and hydrates and solvates thereof. Compound 1 mono(citrate) is formed by, for example, the combination of Compound 1 (free base) and one molecular equivalent of citric acid and can be represented as shown below.

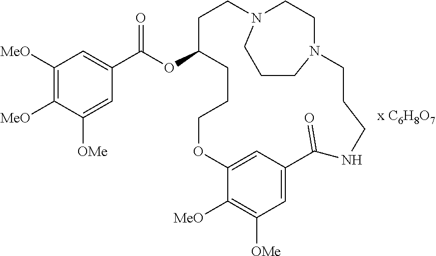 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to Compound 1 di(citrate) and hydrates and solvates thereof. Compound 1 di(citrate) is formed by, for example, the combination of Compound 1 (free base) and two molecular equivalents of citric acid and can be represented as shown below.

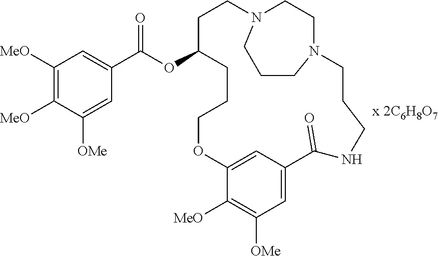 [SMILES: COc1cc(C(=O)O[C@@H]2CCCOc3cc(cc(OC)c3OC)C(=O)NCCCN3CCCN(CC2)CC3)cc(OC)c1OC]

In one aspect, the present disclosure relates to amorphous Compound 1 citrate and hydrates and solvates thereof. In one aspect, the present disclosure relates to amorphous Compound 1 mono(citrate) and hydrates and solvates thereof. In one aspect, the present disclosure relates to amorphous Compound 1 di(citrate) and hydrates and solvates thereof.

In one aspect, the present disclosure relates to Compound 1 maleate and hydrates and solvates thereof. Compound 1 maleate is formed by, for example, the combination of Compound 1 (free base) and maleic acid and can be represented as shown below where n is a number greater than zero.

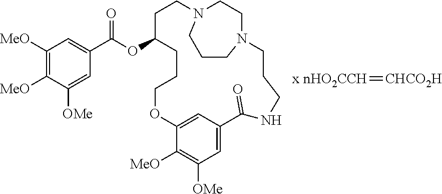

In one aspect, the present disclosure relates to Compound 1 mono(maleate) and hydrates and solvates thereof. Compound 1 mono(maleate) is formed by, for example, the combination of Compound 1 (free base) and one molecular equivalent of maleic acid and can be represented as shown below.

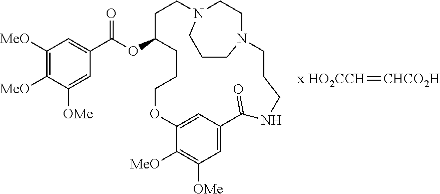

In one aspect, the present disclosure relates to Compound 1 di(maleate) and hydrates and solvates thereof. Compound 1 di(maleate) is formed by, for example, the combination of Compound 1 (free base) and two molecular equivalents of maleic acid and can be represented as shown below.

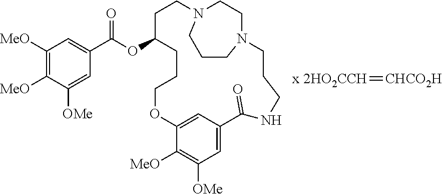

In one aspect, the present disclosure relates to amorphous Compound 1 maleate and hydrates and solvates thereof. In one aspect, the present disclosure relates to amorphous Compound 1 mono(maleate) and hydrates and solvates thereof. In one aspect, the present disclosure relates to amorphous Compound 1 di(maleate) and hydrates and solvates thereof.

In one aspect, the present disclosure relates to a substantially crystalline salt of Compound 1 and hydrates and solvates thereof. In some embodiments, the substantially crystalline salt or hydrate or solvate thereof is more than 50% crystalline. In some embodiments, the substantially crystalline salt or hydrate or solvate thereof is more than about 60% crystalline. In some embodiments, the substantially crystalline salt or hydrate or solvate thereof is more than about 75% crystalline. In some embodiments, the substantially crystalline salt or hydrate or solvate thereof is more than about 90% crystalline. In some embodiments, the substantially crystalline salt or hydrate or solvate thereof is more than about 95% crystalline. In some embodiments, the substantially crystalline salt or hydrate or solvate thereof is more than about 99% crystalline.

In some embodiments, the substantially crystalline salt of Compound 1 is a salt selected from Compound 1 hydrogen chloride, Compound 1 hydrogen sulfate, Compound 1 hydrogen phosphate, Compound 1 tosylate, Compound 1 mesylate, Compound 1 benzenesulfonate, Compound 1 L-tartrate, Compound 1 fumarate, Compound 1 citrate, Compound 1 maleate, Compound 1 L-malate, Compound 1 benzoate, and hydrates and solvates thereof.

In some embodiments, the substantially crystalline salt or hydrate or solvate thereof of Compound 1 is not a hexafluorophosphate salt of Compound 1 or a hydrate or solvate thereof.

### (2) Crystalline Compound 1 (Free Base)

In one aspect, the present disclosure relates to crystalline Compound 1 (free base) and hydrates and solvates thereof.

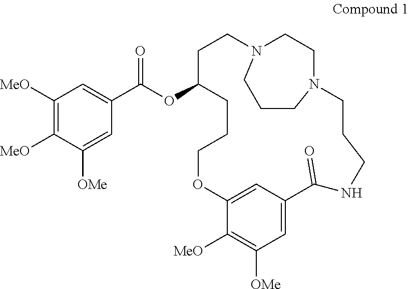 [SMILES: COc1cc(C(=O)O[C@@H]2CCCOc3cc(cc(OC)c3OC)C(=O)NCCCN3CCCN(CC2)CC3)cc(OC)c1OC]

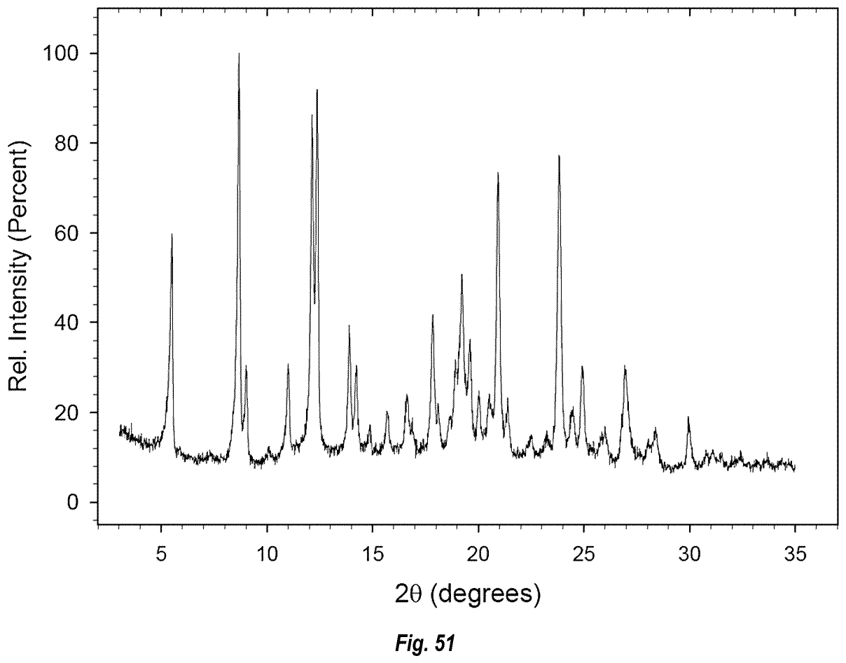

In some embodiments, the crystalline Compound 1 or hydrate or solvate thereof has an X-ray powder diffraction pattern substantially the same as that of FIG. 51.

In one aspect, the present disclosure relates to crystalline Form 1 Compound 1 and hydrates and solvates thereof.

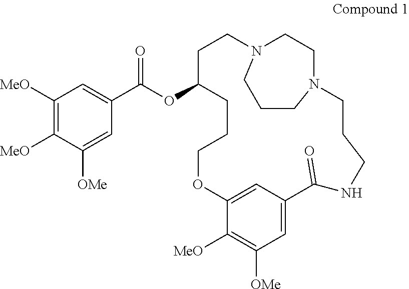 [SMILES: COc1cc(C(=O)O[C@@H]2CCCOc3cc(cc(OC)c3OC)C(=O)NCCCN3CCCN(CC2)CC3)cc(OC)c1OC]

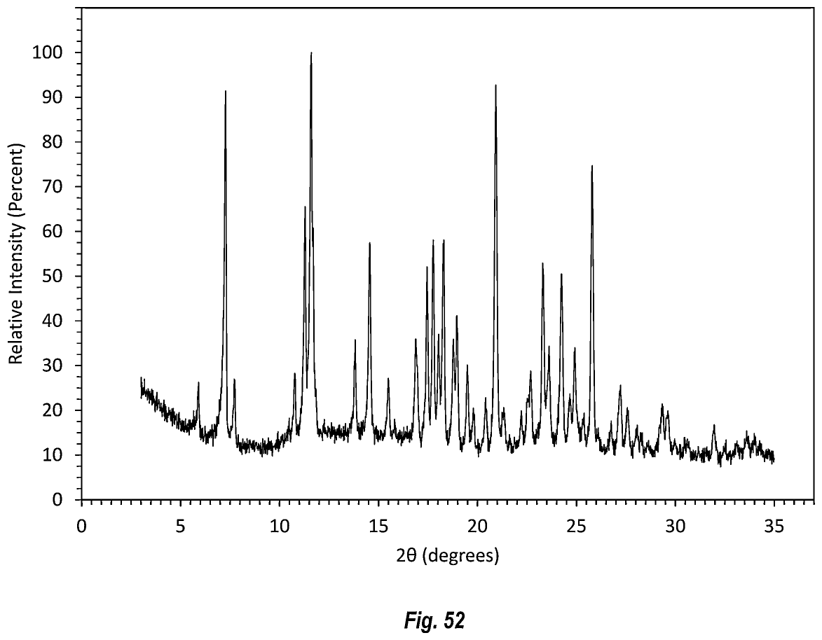

In some embodiments, the crystalline Compound 1 or hydrate or solvate thereof has an X-ray powder diffraction pattern substantially the same as that of FIG. 52.

In one aspect, the present disclosure relates to crystalline Form 2 Compound 1 and hydrates and solvates thereof.

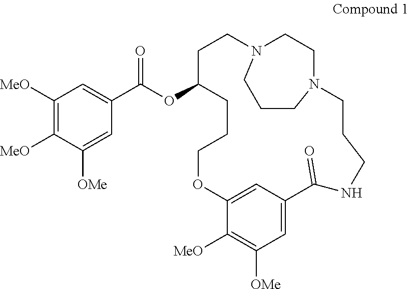 [SMILES: COc1cc(C(=O)O[C@@H]2CCCOc3cc(cc(OC)c3OC)C(=O)NCCCN3CCCN(CC2)CC3)cc(OC)c1OC]

### III. Compositions, Dosage Forms, and Kits

In one aspect, the present disclosure relates to compositions, dosage forms, and kits comprising one or more salts or crystalline complexes of Compound 1, or hydrates or solvates thereof.

### (1) Compositions

In one aspect, the present disclosure provides a composition comprising one or more salts of Compound 1 or a hydrate or solvate thereof. In some embodiments, the composition comprises two or more crystalline salts of Compound 1 or hydrates or solvates thereof.

In some embodiments, the composition comprises two or more of salts or hydrates or solvates thereof selected from Compound 1 hydrogen chloride, Compound 1 hydrogen sulfate, Compound 1 hydrogen phosphate, Compound 1 tosylate, Compound 1 mesylate, Compound 1 benzenesulfonate, Compound 1 L-tartrate, Compound 1 fumarate, Compound 1 citrate, Compound 1 maleate, Compound 1 L-malate, Compound 1 benzoate, and hydrates and solvates thereof. In some embodiments, at least one of the two or more salts of Compound 1 or hydrates or solvates thereof is crystalline.

In one aspect, the present disclosure provides a pharmaceutical composition comprising a salt of Compound 1 or hydrate or solvate thereof and at least one pharmaceutically acceptable excipient. In some embodiments, the salt of Compound 1 or hydrate or solvate thereof is crystalline. In some embodiments, the salt or hydrate or solvate thereof is selected from Compound 1 hydrogen chloride, Compound 1 hydrogen sulfate, Compound 1 hydrogen phosphate, Compound 1 tosylate, Compound 1 mesylate, Compound 1 benzenesulfonate, Compound 1 L-tartrate, Compound 1 fumarate, Compound 1 citrate, Compound 1 maleate, Compound 1 L-malate, Compound 1 benzoate, and hydrates and solvates thereof. In some embodiments, the salt or hydrate or solvate thereof is not a hexafluorophosphate salt of Compound 1 or hydrate or solvate thereof.

In one aspect, the present disclosure provides a composition comprising one or more Compound 1 hydrogen sulfates or hydrates or solvates thereof. In some embodiments, the composition comprises two or more crystalline Compound 1 hydrogen sulfates or hydrates or solvates thereof.

In some embodiments, the composition comprises one or more of crystalline Form 1 Compound 1 di(hydrogen sulfate), crystalline Form 2 Compound 1 di(hydrogen sulfate) trihydrate, crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), and hydrates and solvates thereof.

In some embodiments, at least one of the crystalline Form 1 Compound 1 di(hydrogen sulfate), crystalline Form 2 Compound 1 di(hydrogen sulfate) trihydrate, crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), or hydrate or solvate thereof is substantially crystalline.

In some embodiments, at least one of the crystalline Form 1 Compound 1 di(hydrogen sulfate), crystalline Form 2 Compound 1 di(hydrogen sulfate) trihydrate, crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), or hydrate or solvate thereof is more than about 50% crystalline.

In some embodiments, at least one of the crystalline Form 1 Compound 1 di(hydrogen sulfate), crystalline Form 2 Compound 1 di(hydrogen sulfate) trihydrate, crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), or hydrate or solvate thereof is more than about 90% crystalline.

In some embodiments, at least one of the crystalline Form 1 Compound 1 di(hydrogen sulfate), crystalline Form 2 Compound 1 di(hydrogen sulfate) trihydrate, crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), or hydrate or solvate thereof is more than about 95% crystalline.

In some embodiments, the composition comprises crystalline Form 3 Compound 1 di(hydrogen sulfate) or a hydrate or solvate thereof and crystalline Form 9 Compound 1 di(hydrogen sulfate) or a hydrate or solvate thereof.

In some embodiments, the composition comprises substantially crystalline Form 2 Compound 1 di(hydrogen sulfate) trihydrate, further comprising one or more of crystalline Form 1 Compound 1 di(hydrogen sulfate), crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), and hydrates and solvates thereof.

In some embodiments, the substantially crystalline Form 2 Compound 1 di(hydrogen sulfate) trihydrate is more than about 50% crystalline. In some embodiments, the substantially crystalline Form 2 Compound 1 di(hydrogen sulfate) trihydrate is more than about 90% crystalline. In some embodiments, the substantially crystalline Form 2 Compound 1 di(hydrogen sulfate) trihydrate is more than about 95% crystalline.

In some embodiments, the composition comprises substantially crystalline Form 4 Compound 1 mono(hydrogen sulfate) or a hydrate or solvate thereof, further comprising one or more of crystalline Form 8 Compound 1 mono(hydrogen sulfate), crystalline Form 10 Compound 1 mono(hydrogen sulfate), and hydrates and solvates thereof. In some embodiments, the substantially crystalline Form 4 Compound 1 mono(hydrogen sulfate) is more than about 50% crystalline. In some embodiments, the substantially crystalline Form 4 Compound 1 mono(hydrogen sulfate) is more than about 90% crystalline. In some embodiments, the substantially crystalline Form 4 Compound 1 mono(hydrogen sulfate) is more than about 95% crystalline.

In some embodiments, the composition comprising amorphous Compound 1 di(hydrogen sulfate) or a hydrate or solvate thereof and one or more of crystalline Form 1 Compound 1 di(hydrogen sulfate), crystalline Form 2 Compound 1 di(hydrogen sulfate) trihydrate, crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), and hydrates and solvates thereof.

In some embodiments, the composition comprising amorphous Compound 1 mono(hydrogen sulfate) or a hydrate or solvate thereof and one or more of crystalline Form 4 Compound 1 mono(hydrogen sulfate), crystalline Form 8 Compound 1 mono(hydrogen sulfate), crystalline Form 10 Compound 1 mono(hydrogen sulfate), and hydrates and solvates thereof.

In one aspect, the present disclosure provides a pharmaceutical composition comprising: a Compound 1 hydrogen sulfate or hydrate or solvate thereof as described herein or a composition as described herein, and at least one pharmaceutically acceptable excipient. In some embodiments, the Compound 1 hydrogen sulfate or hydrate or solvate thereof is Compound 1 di(hydrogen sulfate) or a hydrate or solvate thereof. In some embodiments, the Compound 1 hydrogen sulfate is Compound 1 mono(hydrogen sulfate) or a hydrate or solvate thereof. In some embodiments, the Compound 1 hydrogen sulfate or hydrate or solvate thereof is crystalline.

In some embodiments, the present disclosure provides a pharmaceutical composition comprising: a Compound 1 hydrogen sulfate or hydrate or solvate thereof chosen from crystalline Form 1 Compound 1 di(hydrogen sulfate), crystalline Form 2 Compound 1 di(hydrogen sulfate) trihydrate, crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), and hydrates and solvates thereof, and at least one pharmaceutically acceptable excipient. In some embodiments, the Compound 1 hydrogen sulfate or hydrate or solvate thereof is crystalline Form 2 Compound 1 di(hydrogen sulfate) trihydrate. In some embodiments, the Compound 1 hydrogen sulfate or hydrate or solvate thereof is crystalline Form 3 Compound 1 di(hydrogen sulfate) or a hydrate or solvate thereof.

In some embodiments, the present disclosure provides a pharmaceutical composition comprising: a Compound 1 hydrogen sulfate or hydrate or solvate thereof chosen from crystalline Form 4 Compound 1 mono(hydrogen sulfate), crystalline Form 8 Compound 1 mono(hydrogen sulfate), crystalline Form 10 Compound 1 mono(hydrogen sulfate), and hydrates and solvates thereof, and at least one pharmaceutically acceptable excipient. In some embodiments, the Compound 1 hydrogen sulfate or hydrate thereof is chosen from crystalline Form 4 Compound 1 mono(hydrogen sulfate) and hydrates and solvates thereof.

In some embodiments, the composition, including pharmaceutical compositions, may be administered orally or configured to be delivered as any effective conventional dosage unit forms, including immediate, slow and timed-release oral preparations, parenterally, topically, nasally, ophthalmically, optically, sublingually, rectally, vaginally, and the like. In some embodiments, the pharmaceutical composition is an immediate release composition.

In some embodiments, the at least one pharmaceutically acceptable excipient is chosen from pharmaceutically acceptable excipients include cosolvents, binders, antioxidants, surfactants, wetting agents, dissolution aids, emulsifying agents, buffering agents, pH modifying agents, preserving agents (or preservating agents), isotonifiers, stabilizing agents, granulating agents or binders, precipitation inhibitors, lubricants, disintegrants, glidants, diluents or fillers, adsorbents, dispersing agents, suspending agents, bulking agents, release agents, sweetening agents, flavoring agents, coatings, gelatins, carbohydrates such as lactose, amylose or starch, fatty acid esters, hydroxypropylmethylcellulose, polyvinyl pyrrolidine, and colors, and the like. For examples of excipients, see Martin, Remington's Pharmaceutical Sciences, 15th Ed., Mack Publ. Co., Easton, PA (1975) or Rowe, Shesky, and Quinn, Handbook of Pharmaceutical Excipients, 6th Ed. Pharmaceutical Press, London, UK (2009). The various classes of excipient examples above are not rigid categories and may at times be characterized in more than one category.

Examples of diluents or fillers include, but are not limited to, a sugar (e.g., mannitol, lactose, sorbitol, lactitol, erythritol, sucrose, fructose, glucose, agarose, maltose, isomalt, polydextrose, and combinations thereof), an inorganic material (e.g., dibasic calcium phosphate, hydroxyapatite, sodium carbonate, sodium bicarbonate, calcium carbonate, calcium sulfate, magnesium carbonate, magnesium oxide, bentonite, kaolin), calcium lactate, a starch (e.g., a pregelatinized starch), a microcrystalline cellulose, a silicified microcrystalline cellulose, a polysaccharide, a cellulose (e.g., a hydroxypropylcellulose, a hypromellose, a carboxymethylcellulose, a methylcellulose, a hydroxypropylmethylcellulose, a hydroxyethylcellulose), a dextrin, a maltodextrin, an alginate, a collagen, a polyvinylpyrrolidone, a polyvinylacrylate, polyethylene oxide, and polyethylene glycol. Sugar is defined herein to include sugar alcohols.

Examples of adsorbents include, but are not limited to silicon dioxide, purified aluminum silicate and the like.

Examples of disintegrants include, but are not limited to, alginic acid, an alginate, primogel, a cellulose (e.g., hydroxypropylcellulose), polacrillin potassium, sodium starch glycolate, sodium croscarmellose, a polyplasdone (e.g., a crospovidone), and a starch (e.g., corn starch, pregelatinized starch, hydroxypropyl starch, and carboxymethyl starch).

Examples of binders include, but are not limited to, a hydroxypropylcellulose, hydroxyethylcellulose, a hydroxypropylmethylcellulose (e.g., a low viscosity hydroxypropylmethylcellulose), a sugar, a polyvinylpyrrolidone, a polyvinyl alcohol, a polyvinyl acetate, a polydextrose, a chitosan, a carrageenan, carbophil, a microcrystalline cellulose, gum tragacanth, guar gum, gellan gum, gelatin, and a starch (e.g., corn starch).

Examples of wetting agents include, but are not limited to, a poloxamer (e.g., poloxamer 407), sodium dodecyl sulfate, sodium lauryl sulfate (SLS), sodium stearyl fumarate (SSF), a polydimethylsiloxane, a polysorbate (e.g., polyoxyethylene 20 sorbitan mono-oleate (Tween® 20)), sorbitan monooleate, sorbitan trioleate, sorbitan laurate, sorbitan stearate, sorbitan monopalmitate, lecithin, sodium taurocholate, ursodeoxycholate, polyethoxylated castor oil, cetyl trimethylammonium bromide, nonoxynol, \acute{α}-tocopherol polyethylene glycol 1000 succinate, and docusate sodium. In some embodiments, the wetting agent is sodium lauryl sulphate. Further examples of wetting and/or dissolution aids include certain cosolvents including, but not limited to caprylic acid, polyethylene glycol (PEG), propylene glycol, ethanol, dimethylsulfoxide, dimethylacetamide, dimethylisosorbide and mixtures thereof.

Examples of antioxidants include, but are not limited to butylated hydroxytoluene (BHT), butylated hydroxyanisole (BHA), citric acid, sodium metabisulfite, ascorbic acid, methionine and vitamin E. In some embodiments, the antioxidant is BHT.

Examples of surfactants include, but are not limited to polyethylene glycols, polyoxyethylene sorbitan fatty acid esters, sorbitan esters, sodium docusate, sodium lauryl sulfate, polysorbates (20, 80, etc.), poloxamers (188, 407 etc.), pluronic polyols, polyoxyethylene sorbitan monoethers (TWEEN®-20, TWEEN®-80, etc.), vitamin E TPGS (Vitamin E polyethylene glycol succinate), cremophor RH40 (polyoxyl 40 hydrogenated castor oil), cremophor EL (polyoxyl 35 hydrogenated castor oil), polyethylene glycol 660 12-monostearate, solutol HS15 (Polyoxyethylated 12-hydroxystearic acid), labrasol (caprylocaproyl polyoxyl-8 glycerides), labrafil M1944 (Oleoyl polyoxyl-6 glycerides).

Examples of glidants include, but are not limited to, colloidal silicon dioxide, colloidal anhydrous silica (also known as colloidal silicon dioxide), talc, kaolin, bentonite, and activated carbon/charcoal.

Examples of lubricants and glidants include, but are not limited to, a wax, a glyceride, a light mineral oil, a polyethylene glycol, sodium stearyl fumarate, magnesium stearate, stearic acid, hydrogenated oil (e.g., hydrogenated vegetable oil), an alkyl sulfate, sodium benzoate, sodium acetate, glyceryl behenate, palmitic acid, and coconut oil.

Examples of emulsifying agents include, but are not limited to, carbomer, carrageenan, lanolin, lecithin, mineral oil, oleic acid, oleyl alcohol, pectin, poloxamer, polyoxyethylene sorbitan fatty acid esters, sorbitan esters, triethanolamine, propylene glycol monolaurate, propylene glycol dilaurate, propylene glycol monocaprylate. In some embodiments emulsifying agents are for example poloxamer, propylene glycol monolaurate, propylene glycol dilaurate, and propylene glycol monocaprylate.

Examples of buffering agents that are used to help to maintain the pH in the range that approximates physiological conditions include both organic and inorganic acids and salts thereof, such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Examples of pH modifiers include, but are not limited to sodium hydroxide, sodium bicarbonate, magnesium oxide, potassium hydroxide, meglumine, sodium carbonate, citric acid, tartaric acid, ascorbic acid, fumaric acid, succinic acid and malic acid.

Examples of preservatives agents added to retard microbial include, but are not limited to phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol.

Examples of isotonifiers (sometimes known as “stabilizers”) include, but are not limited to polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall or helps to inhibit the precipitation, particle growth or agglomeration of the active ingredient. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc.; organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone; cellulose derivatives such as hydroxypropylmethylcellulose, hydroxypropylmethylcellulose phthalate or hydroxypropylmethylcellulose acetate succinate; carboxymethylcellulose (Na/Ca); monosaccharides, such as xylose, mannose, fructose, glucose; disaccharides such as lactose, maltose, sucrose and trisaccacharides such as raffinose; polysaccharides such as dextran; polyethylene glycol methyl ether-block-poly(D-L-lactide) copolymer; poly(butyl methacrylate-co-(2-dimethylaminoethyl) methacrylate-co-methyl methacrylate) 1:2:1. In some embodiments stabilizers are for example glycerol; polyethylene glycol; polyvinylpyrrolidone; cellulose derivatives such as hydroxypropylmethylcellulose, hydroxypropylmethylcellulose phthalate or hydroxypropylmethylcellulose acetate succinate; carboxymethylcellulose (Na/Ca); polyethylene glycol methyl ether-block-poly(D-L-lactide) copolymer; and poly(butyl methacrylate-co-(2-dimethylaminoethyl) methacrylate-co-methyl methacrylate) 1:2:1.

Examples of granulating agent/binder(s) include, but are not limited to starch, gums (inclusive of natural, semisynthetic and synthetic), microcrystalline cellulose, ethyl cellulose, methylcellulose, hydroxypropylcellulose, liquid glucose polymers such as povidone, polyvinylpyrrolidone polyvinylacetate copolymer and the like. In some embodiments granulating agents are for example methylcellulose, hydroxypropylcellulose, povidone and polyvinylpyrrolidone polyvinylacetate copolymer.

Examples of precipitation inhibitors include, but are not limited to, water soluble derivatives of cellulose including hydroxypropylmethylcellulose and methylcellulose, and water soluble polymers such as polyvinylpyrrolidone or polyvinylpyrrolidone polyvinylacetate copolymer. In some embodiments the precipitation inhibitor is hydroxypropylmethylcellulose.

Examples of colorants include, but are not limited to, titanium dioxide, aluminum lakes, iron oxides and carbon black.

Examples of coatings include but are not limited to, a film forming polymer (e.g., a hypromellose, a methyl cellulose, an ethylcellulose, cellulose acetate, a hydroxypropylmethyl cellulose, a hydroxypropyl cellulose, hydroxypropylmethyl cellulose acetate succinate, cellulose acetate phthalate, a polyvinylpyrrolidone, polyvinyl alcohol, a Eudragit/acrylate) and a plasticizer (e.g., triacetin, polyethylene glycol, propylene glycol).

In some embodiments, the at least one pharmaceutically acceptable excipient is a diluent or filler. In some embodiments, the at least one pharmaceutically acceptable excipient is a disintegrant. In some embodiments, the at least one pharmaceutically acceptable excipient is a glidant. In some embodiments, the at least one pharmaceutically acceptable excipient is a lubricant. In some embodiments, the pharmaceutical compositions disclosed herein comprise a diluent or filler, a disintegrant, a glidant, and a lubricant.

In some embodiments, the diluent or filler is a pregelatinized starch. In some embodiments, the disintegrant is a crospovidone. In some embodiments, the glidant is colloidal anhydrous silica/colloidal silicon dioxide. In some embodiments, the lubricant is magnesium stearate. In some embodiments, the pharmaceutical compositions disclosed herein comprise a pregelatinized starch, a crospovidone, colloidal anhydrous silica/colloidal silicon dioxide, and magnesium stearate.

In some embodiments, the pharmaceutical composition disclosed herein comprises about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, or about 15% w/w of a salt of Compound 1 or a hydrate or solvate thereof. In some embodiments, the pharmaceutical composition disclosed herein comprises about 1% w/w of salt of Compound 1 or a hydrate or solvate thereof. In some embodiments, the pharmaceutical composition disclosed herein comprises about 2% w/w of salt of Compound 1 or a hydrate or solvate thereof. In some embodiments, the pharmaceutical composition disclosed herein comprises about 14% w/w of salt of Compound 1 or a hydrate or solvate thereof. In some embodiments, the pharmaceutical composition disclosed herein comprises about 15% w/w of salt of Compound 1 or a hydrate or solvate thereof.

In some embodiments, the pharmaceutical composition disclosed herein comprises about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, or about 15% w/w of Compound 1 hydrogen sulfate, crystalline Compound 1, or a hydrate or solvate thereof. In some embodiments, the pharmaceutical composition disclosed herein comprises about 1% w/w of Compound 1 hydrogen sulfate, crystalline Compound 1, or a hydrate or solvate thereof. In some embodiments, the pharmaceutical composition disclosed herein comprises about 2% w/w of Compound 1 hydrogen sulfate, crystalline Compound 1, or a hydrate or solvate thereof. In some embodiments, the pharmaceutical composition disclosed herein comprises about 14% w/w of Compound 1 hydrogen sulfate, crystalline Compound 1, or a hydrate or solvate thereof. In some embodiments, the pharmaceutical composition disclosed herein comprises about 15% w/w of Compound 1 hydrogen sulfate, crystalline Compound 1, or a hydrate or solvate thereof.

### (2) Oral Dosage Forms

In one aspect, the present disclosure provides a dosage form comprising a pharmaceutical composition as described herein. In some embodiments, the dosage form is a solid dosage form. In some embodiments, the dosage form is an oral dosage form. In some embodiments, the dosage form is a solid oral dosage form. In some embodiments, the solid oral dosage form is chosen from tablets, capsules, granules, powders, minitabs, sachets, stickpacks, reconstitutable powders, dry powder inhalers, lozenges, and chewables.

In some embodiments, the dosage form is a capsule. In some embodiments, the capsule is a gel capsule. In some embodiments, the capsule is a hard gel capsule. In some embodiments, the size of the capsule is chosen from 000, 00, 0, 1, 2, 3, 4, and 5. In some embodiments, the capsule contains 100 mg of the pharmaceutical composition. In some embodiments, the capsule contains 150 mg of the pharmaceutical composition. In some embodiments, the capsule contains 200 mg of the pharmaceutical composition. In some embodiments, the capsule contains 250 mg of the pharmaceutical composition. In some embodiments, the capsule contains 300 mg of the pharmaceutical composition.

### (3) Kits

In one aspect, the present disclosure provides a kit of parts comprising: a salt of Compound 1 or a hydrate or solvate thereof; crystalline Compound 1 or a hydrate or solvate thereof; a composition as described herein; or a dosage form as described herein. In one aspect, the present disclosure provides a kit of parts comprising Compound 1 hydrogen sulfate or a hydrate or solvate thereof.

In one aspect, the present disclosure provides a kit of parts comprising: (1) a first part comprising a salt of Compound 1 or a hydrate or solvate thereof; crystalline Compound 1 or a hydrate or solvate thereof; a composition as described herein; or a dosage form as described herein; and (2) a second part comprising an adenosine receptor antagonist. In one aspect, the present disclosure provides a kit of parts comprising: (1) a first part comprising Compound 1 hydrogen sulfate or a hydrate or solvate thereof; and (2) a second part comprising an adenosine receptor antagonist.

In some embodiments, the adenosine receptor antagonist is an A2A or A2B receptor antagonist. In some embodiments, the adenosine receptor antagonist is any of the adenosine receptor antagonist described herein. For example, as discussed below in more detail, an adenosine receptor antagonist described herein is (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (also known as inupadenant) or a pharmaceutically acceptable salt or solvate thereof. In some embodiments, the A_{2A}R antagonist is a hydrochloride salt of (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a solvate thereof. See, e.g., WO 2023/059817, the entire content of which is incorporated herein by reference.

In some embodiments, the hydrochloride salt of (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a solvate thereof is amorphous. See, e.g., WO 2023/059817 at Example 6.

In some embodiments, the hydrochloride salt of (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a solvate thereof is crystalline. In some embodiments, the crystalline hydrochloride salt of (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a solvate thereof is Form 1. See, e.g., WO 2023/059817 at Example 2. In some embodiments, the crystalline hydrochloride salt of (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a solvate thereof is Form 2. See, e.g., WO 2023/059817 at Examples 3-4.

### IV. Methods and Uses

In one aspect, the present disclosure relates to methods and uses of a salt or crystalline complex of Compound 1, or a hydrate or solvate thereof. In one aspect, the present disclosure relates to methods and uses of Compound 1 hydrogen sulfate, crystalline Compound 1, and hydrates and solvates thereof.

In one aspect, the present disclosure provides a method of inhibiting ENT1 in a patient need thereof, comprising: administering to said patient an effective amount of a salt of Compound 1 or hydrate or solvate thereof; an effective amount of crystalline Compound 1 or a hydrate or solvate thereof; an effective amount of a composition as described herein; or a dosage form as described herein. In one aspect, the present disclosure provides a method of inhibiting ENT1 in a patient need thereof. In some embodiments, the method comprises: administering to said patient an effective amount of Compound 1 hydrogen sulfate or a hydrate or solvate thereof.

In one aspect, the present disclosure provides a use of a salt of Compound 1 or hydrate or solvate; crystalline Compound 1 or a hydrate or solvate thereof; a composition as described herein; or a dosage form as described herein, for the manufacture of a medicament for inhibiting ENT1. In one aspect, the present disclosure provides a use of Compound 1 hydrogen sulfate or a hydrate or solvate thereof for the manufacture of a medicament for inhibiting ENT1.

In one aspect, the present disclosure provides a salt of Compound 1 or hydrate or solvate thereof; crystalline Compound 1 or a hydrate or solvate thereof; a composition as described herein; or a dosage form as described herein for use as a medicament. In one aspect, the present disclosure provides Compound 1 hydrogen sulfate or a hydrate or solvate thereof.

In one aspect, the present disclosure provides a method of treating cancer in a patient need thereof, comprising: administering to said patient an effective amount of a salt of Compound 1 or hydrate or solvate thereof; an effective amount of crystalline Compound 1 or a hydrate or solvate thereof; an effective amount of a composition as described herein; or a dosage form as described herein. In one aspect, the present disclosure provides a method of treating cancer in a patient need thereof, comprising: administering to said patient an effective amount of Compound 1 hydrogen sulfate or a hydrate or solvate thereof.

In one aspect, the present disclosure provides a use of a salt of Compound 1 or hydrate or solvate thereof; crystalline Compound 1 or a hydrate or solvate thereof; a composition as described herein; or a dosage form as described herein, for the manufacture of a medicament for treating cancer. In one aspect, the present disclosure provides a use of Compound 1 hydrogen sulfate or a hydrate or solvate thereof for the manufacture of a medicament for treating cancer.

In one aspect, the present disclosure provides salt of Compound 1 or hydrate or solvate thereof; crystalline Compound 1 or a hydrate or solvate thereof; a composition as described herein; or a dosage form as described herein, for use in a method of treating cancer. In one aspect, the present disclosure provides Compound 1 hydrogen sulfate or a hydrate or solvate thereof for use in a method of treating cancer.

In one aspect, the present disclosure provides a method of treating cancer in a patient need thereof, comprising: administering to said patient (1) an effective amount of a salt of Compound 1 or hydrate or solvate thereof; an effective amount of crystalline Compound 1 or a hydrate or solvate thereof; an effective amount of a composition as described herein; or a dosage form as described herein; and (2) an adenosine receptor antagonist. In one aspect, the present disclosure provides a method of treating cancer in a patient need thereof, comprising: administering to said patient (1) an effective amount of Compound 1 hydrogen sulfate or a hydrate or solvate thereof; and (2) an adenosine receptor antagonist.

In one aspect, the present disclosure provides a use of a combination of (1) a salt of Compound 1 or hydrate or solvate thereof; crystalline Compound 1 or a hydrate or solvate thereof; a composition as described herein; or a dosage form as described herein; and (2) an adenosine receptor antagonist for the manufacture of a medicament for treating cancer. In one aspect, the present disclosure provides a use of a combination of (1) Compound 1 hydrogen sulfate or a hydrate or solvate thereof; and (2) an adenosine receptor antagonist for the manufacture of a medicament for treating cancer.

In some embodiments, the adenosine receptor antagonist is an A2A or A2B receptor antagonist.

In some embodiments, the A_{2A}R antagonist is a thiocarbamate disclosed in WO 2018/178338 and WO 2023/059817, both of which are incorporated herein by reference. In some embodiments, the A_{2A}R antagonist is a compound of formula (I):

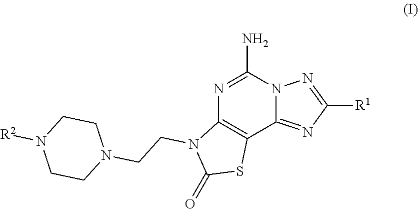

or a pharmaceutically acceptable salt or solvate thereof,
wherein
R¹ represents a 5- or 6-membered heteroaryl or 5- or 6-membered aryl, wherein the heteroaryl or aryl groups are optionally substituted by one or more substituent selected from C₁-C₆ alkyl (preferably methyl) and halo (preferably fluoro or chloro); preferably R¹ represents a 5-membered heteroaryl; more preferably R¹ a represents furyl;
R² represents a 6-membered aryl or 6-membered heteroaryl,
wherein the heteroaryl or aryl groups are optionally substituted by one or more substituent selected from halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino and alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl alkylsulfonyl and alkylsulfonealkyl;
or the heteroaryl or aryl groups are optionally substituted with two substituents that form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, the A_{2A}R antagonist is a compound of Formula (Ia):

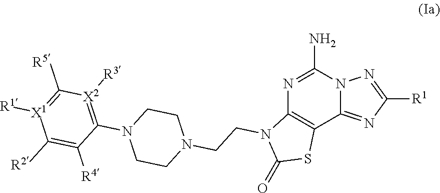

or a pharmaceutically acceptable salt or solvate thereof,
wherein:
R¹ represents a 5- or 6-membered heteroaryl or 5- or 6-membered aryl, wherein the heteroaryl or aryl groups are optionally substituted by one or more substituent selected from C₁-C₆ alkyl (preferably methyl) and halo (preferably fluoro or chloro); preferably R¹ represents a 5-membered heteroaryl; more preferably R¹ represents a furyl;
X¹ and X² represent each independently C or N;
R¹′ is absent when X¹ is N; or when X¹ is C, R¹′ represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl;
said R¹′ being optionally substituted where appropriate by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
R²′ represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl;
said R²′ being optionally substituted where appropriate by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
or R¹′ and R²′ form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;

R³′ is absent when X² is N; or when X² is C, R³′ represents H or halo, preferably H or F;
R⁴′ represents H or halo, preferably H or F; and
R⁵′ represents H or halo, preferably H or F.

In some embodiments, the A_{2A}R antagonist is a compound of Formula (Ia-1):

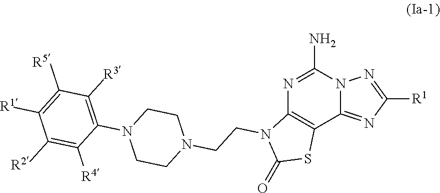

or a pharmaceutically acceptable salt or solvate thereof, wherein R¹, R¹′, R²′, R³′, R⁴′ and R⁵′ are as defined in Formula (Ia).

In some embodiments, an A_{2A}R antagonist is a compound of Formula (Ia-1a):

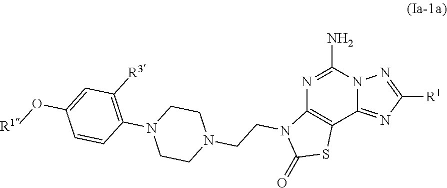

or a pharmaceutically acceptable salt or solvate thereof,
wherein:
R¹ and R³′ are as defined in Formula (Ia); and
R¹″ represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In some embodiments, the A_{2A}R antagonist is a compound of Formula (Ia-1b):

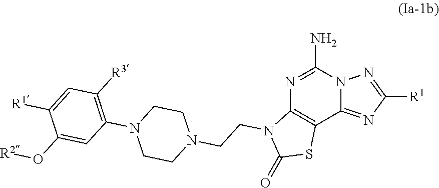

or a pharmaceutically acceptable salt or solvate thereof,
wherein:
R¹ and R³′ are as defined in Formula (Ia);
R¹′ represents H or halo, preferably H or F; and
R²″ represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In some embodiments, the A_{2A}R antagonist is a compound of Formula (Ia-1c) or (Ia-1d):

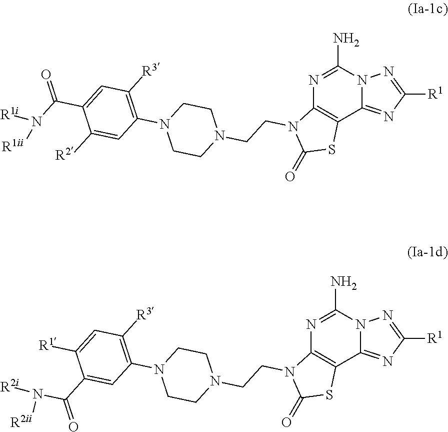

or a pharmaceutically acceptable salt or solvate thereof,
wherein:
R¹ and R³′ are as defined in Formula (Ia);
R¹′ represents H or halo, preferably H or F;
R²′ represents H or halo, preferably H or F;
R¹ⁱ and R¹ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl; and
R²ⁱ and R²ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl.

In some embodiments, the A_{2A}R antagonist is a compound of Formulae (Ia-2) or (IIa-3):

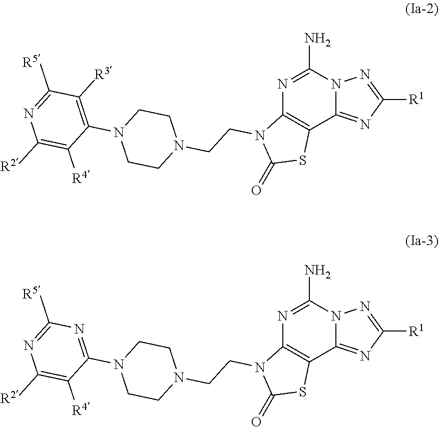

or a pharmaceutically acceptable salt or solvate thereof,
wherein R¹, R²′, R³′, R⁴ and R⁵′ are as defined in Formula (Ia).

In some embodiments, the A_{2A}R antagonist is a compound chosen from:
3-(2-(4-(4-((1H-1,2,3-triazolo-4-yl)methoxy-2-fluorophenyl)piperazine-1-yl)ethyl)-5-amino-(8-(furan-2-yl)thiazolo[5,4-e][1,2,4]triazolo[1,5-c]pyrimidine-2(3H)-one;
5-((4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)methyl)-1,3,4-oxadiazol-2(3H)-one;
5-amino-3-(2-(4-(3-fluoropyridin-4-yl)piperazin-1-yl)ethyl)-8-(furan-2-yl)thiazolo[5,4-e][1,2,4]triazolo[1,5-c]pyrimidin-2(3H)-one;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)acetamide;
(S)-5-amino-3-(2-(4-(2-fluoro-4-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2-fluoro-4-(2-(methylsulfinyl)ethoxy)phenyl)-piperazin-1-yl)ethyl)-8-(furan-2-yl)thiazolo[5,4-e][1,2,4]triazolo[1,5-c]pyrimidin-2(3H)-one;
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-8-(furan-2-yl)-3-(2-(4-(4-(2-hydroxyethoxy)phenyl)piperazin-1-yl)ethyl)thiazolo[5,4-e][1,2,4]triazolo[1,5-c]pyrimidin-2(3H)-one;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)phenoxy)acetic acid;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)phenoxy)acetamide;
5-amino-3-(2-(4-(4-(2,3-dihydroxypropoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(4-(2-aminoethoxy)phenyl)piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-methylbenzamide;
5-amino-8-(furan-2-yl)-3-(2-(4-(4-(2-morpholinoethoxy)phenyl)piperazin-1-yl)ethyl)thiazolo[5,4-e][1,2,4]triazolo[1,5-c]pyrimidin-2(3H)-one;
5-amino-3-(2-(4-(4-(2-(dimethylamino)ethoxy)phenyl)piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzenesulfonamide;
4-(4-(2-(5-amino-8-(furan-2-yl)-2-oxothiazolo[5,4-e][1,2,4]triazolo[1,5-c]pyrimidin-3(2H)-yl)ethyl) piperazin-1-yl)-N-methylbenzenesulfonamide;
5-amino-8-(furan-2-yl)-3-(2-(4-(4-(methylsulfonyl)phenyl)piperazin-1-yl)ethyl)thiazolo[5,4-e][1,2,4]triazolo[1,5-c]pyrimidin-2(3H)-one;
5-amino-8-(furan-2-yl)-3-(2-(4-(4-(methylsulfinyl)phenyl)piperazin-1-yl)ethyl)thiazolo[5,4-e][1,2,4]triazolo[1,5-c]pyrimidin-2(3H)-one;
3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzamide;
5-amino-8-(furan-2-yl)-3-(2-(4-(3-(2-hydroxyethoxy)phenyl)piperazin-1-yl)ethyl)thiazolo[5,4-e][1,2,4]triazolo[1,5-c]pyrimidin-2(3H)-one;
5-amino-3-(2-(4-(2-fluoro-4-(2-oxo-2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(piperidin-4-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(piperazine-1-carbonyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(piperazin-1-ylsulfonyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(methylsulfonyl)phenyl) piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-aminoethyl)-3-fluorobenzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylamino)ethyl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluorobenzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-hydroxyethyl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-3-fluorobenzamide;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)acetic acid;
2-(4-(4-(2-(5-amino-8-(furan-2-yl)-2-oxothiazolo[5,4-e][1,2,4]triazolo[1,5-c]pyrimidin-3(2H)-yl)ethyl) piperazin-1-yl)-3,5-difluorophenoxy) acetic acid;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid;
(S)-2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-2-methylpropanoic acid;
3-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenyl)propanoic acid;
4-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)butanoic acid;
2-(3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,6-difluorophenoxy) acetic acid;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) acetic acid;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorobenzoic acid;
2-((2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)amino)acetamide;
2-((2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)(methyl)amino)acetamide;
5-amino-3-(2-(4-(2-fluoro-4-(piperidin-4-yloxy)phenyl)piperazin-1-yl)ethyl)-8-(furan-2-yl) thiazolo[5,4-e][1,2,4]triazolo[1,5-c]pyrimidin-2(3H)-one;
5-amino-3-(2-(4-(2-fluoro-4-(pyrrolidin-3-yloxy)phenyl)piperazin-1-yl)ethyl)-8-(furan-2-yl)thiazolo[5,4-e][1,2,4]triazolo[1,5-c]pyrimidin-2(3H)-one;
3-(2-(4-(4-((1H-1,2,4-triazol-3-yl)methoxy)-2-fluorophenyl)piperazin-1-yl)ethyl)-5-amino-8-(furan-2-yl)thiazolo[5,4-e][1,2,4]triazolo[1,5-c]pyrimidin-2(3H)-one;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-(methylamino)ethyl) acetamide;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-(dimethylamino)ethyl) acetamide;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-aminoethyl)acetamide;
(R)-2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)acetamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-methyl-N-(2-(methylamino)ethyl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluoro-N-methylbenzamide;
(R)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(1-(dimethylamino) propan-2-yl)-3-fluorobenzamide;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-methyl-N-(2-(methylamino)ethyl) acetamide;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-2-methylpropanoic acid;
(S)-2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) propanoic acid;
(R)-2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) propanoic acid;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-(methylamino)ethyl) acetamide;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-(dimethylamino)ethyl) acetamide;
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-2,4-difluoro-N-methylbenzamide;
4-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) butanoic acid;
3-(2-(4-(5-((1H-tetrazol-5-yl)methoxy)-2,4-difluorophenyl)piperazin-1-yl)ethyl)-5-amino-8-(furan-2-yl)thiazolo[5,4-e][1,2,4]triazolo[1,5-c]pyrimidin-2(3H)-one;
5-amino-3-(2-(4-(2-fluoro-4-((1-methyl-1H-1,2,4-triazol-3-yl)methoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-((1-methyl-1H-1,2,4-triazol-3-yl) methoxy)phenyl)piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methyl (oxetan-3-yl)amino)ethyl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-((2-hydroxyethyl)amino)ethyl)benzamide;
2-amino-N-(2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl) acetamide;
(S)-2-amino-N-(2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)-3-methylbutanamide;
ethyl 2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)acetate;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) acetonitrile;
5-amino-8-(furan-2-yl)-3-(2-(4-(pyridin-4-yl) piperazin-1-yl)ethyl)thiazolo[5,4-e][1,2,4]triazolo[1,5-c]pyrimidin-2(3H)-one;
5-amino-8-(furan-2-yl)-3-(2-(4-(pyrimidin-4-yl)piperazin-1-yl)ethyl)thiazolo[5,4-e][1,2,4]triazolo[1,5-c]pyrimidin-2(3H)-one;
5-amino-3-(2-(4-(2,4-difluoro-5-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(6-fluoro-2-oxoindolin-5-yl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(S-methylsulfonimidoyl)phenyl)piperazin-1-yl)ethyl)-8-(furan-2-yl)thiazolo[5,4-e][1,2,4]triazolo[1,5-c]pyrimidin-2(3H)-one;
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-2,4-difluorobenzamide;
5-amino-3-(2-(4-(5-fluoro-2-methylpyridin-4-yl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3R,4R)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3S,4S)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(2-hydroxy-2-methylpropoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(2-hydroxypropan-2-yl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(3,3,3-trifluoro-2-hydroxypropoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-5-(2-hydroxyethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3R,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3S,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2,4-difluoro-5-((2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-((2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-morpholinoethyl)acetamide;
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(morpholin-3-ylmethyl)benzamide;
5-amino-3-(2-(4-(2-fluoro-4-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3R,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3R,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3S,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-morpholinoethyl)acetamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-morpholinoethyl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(morpholin-3-ylmethyl)benzamide;
5-amino-3-(2-(4-(4-(azetidin-3-yloxy)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((1s,4s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide;
5-amino-3-(2-(4-(2,4-difluoro-5-(1-oxidothiomorpholine-4-carbonyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((1s,4s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
(S)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;
(R)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;
5-amino-3-(2-(4-(2-fluoro-4-(1-oxidothiomorpholine-4-carbonyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo[1,5-c]pyrimidin-2(3H)-one;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-2,4-difluorobenzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-2,4-difluorobenzamide;
5-amino-3-(2-(4-(4-(azetidin-3-yloxy)-2-fluorophenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(5-(azetidin-3-yloxy)-2,4-difluorophenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(3-(methylsulfinyl)propoxy)phenyl)piperazin-1-yl)ethyl)-8-(furan-2-yl)thiazolo[5,4-e][1,2,4]triazolo[1,5-c]pyrimidin-2(3H)-one,
or a pharmaceutically acceptable salt or solvate thereof.

In some embodiments, an A_{2A}R antagonist is selected from the group consisting of:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one,

or a pharmaceutically acceptable salt or solvate thereof.

In some embodiments, an A_{2A}R antagonist is selected from the group consisting of:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one,

and pharmaceutically acceptable salts and solvates thereof.

In some embodiments, the A_{2A}R antagonist is (R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a pharmaceutically acceptable salt or solvate thereof.

In some embodiments, the A_{2A}R antagonist is (+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a pharmaceutically acceptable salt or solvate thereof.

In some embodiments, the A_{2A}R antagonist is (−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a pharmaceutically acceptable salt or solvate thereof.

In some embodiments, the A_{2A}R antagonist is (R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a pharmaceutically acceptable salt or solvate thereof. In some embodiments, the A_{2A}R antagonist is (R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a pharmaceutically acceptable salt thereof. In some embodiments, the A_{2A}R antagonist is (R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one.

In some embodiments, the A_{2A}R antagonist is (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (also known as inupadenant) or a pharmaceutically acceptable salt or solvate thereof. In some embodiments, the A_{2A}R antagonist is (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a pharmaceutically acceptable salt thereof. In some embodiments, the A_{2A}R antagonist is (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one.

In some embodiments, the A_{2A}R antagonist is a hydrochloride salt of (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a solvate thereof. See, e.g., WO 2023/059817.

In some embodiments, the hydrochloride salt of (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a solvate thereof is amorphous. See, e.g., WO 2023/059817 at Example 6.

In some embodiments, the hydrochloride salt of (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a solvate thereof is crystalline. In some embodiments, the crystalline hydrochloride salt of (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a solvate thereof is Form 1. See, e.g., WO 2023/059817 at Example 2. In some embodiments, the crystalline hydrochloride salt of (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a solvate thereof is Form 2. See, e.g., WO 2023/059817 at Examples 3-4.

In some embodiments, the A_{2A}R antagonist is an A_{2A}R antagonist disclosed in WO 2011/121418. For example, the A_{2A}R antagonist is the compound of example 1 of WO 2011/121418, namely 5-bromo-2,6-di-(1H-pyrazol-1-yl)pyrimidin-4-amine, also known as NIR178:

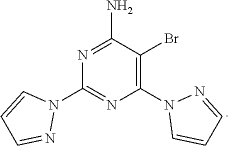 [SMILES: Nc1nc(-n2cccn2)nc(-n2cccn2)c1Br]

In some embodiments, the A_{2A}R antagonist is an A_{2A}R antagonist disclosed in WO 2009/156737. For example, the A_{2A}R antagonist is the compound of example 1S of WO 2009/156737, namely (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine, also known as CPI-444:

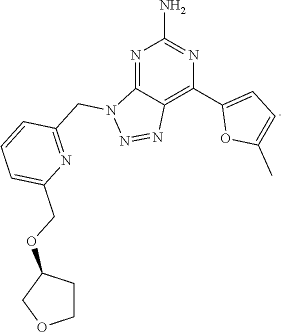 [SMILES: Cc1ccc(-c2nc(N)nc3c2nnn3Cc2cccc(CO[C@H]3CCOC3)n2)o1]

In some embodiments, the A_{2A}R antagonist is an A_{2A}R antagonist disclosed in WO 2011/095626. For example, the A_{2A}R antagonist is the compound (cxiv) of WO 2011/095626, namely 6-(2-chloro-6-methylpyridin-4-yl)-5-(4-fluorophenyl)-1,2,4-triazin-3-amine, also known as AZD4635:

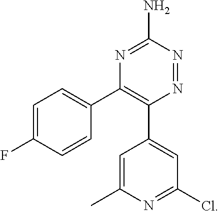 [SMILES: Cc1cc(-c2nnc(N)nc2-c2ccc(F)cc2)cc(Cl)n1]

In some embodiments, the A_{2A}R antagonist is an A_{2A}R antagonist disclosed in WO 2018/136700. For example, the A_{2A}R antagonist is the compound of example 1 of WO 2018/136700, namely 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile, also known as AB928:

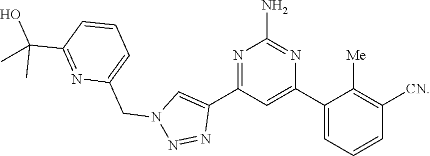 [SMILES: Cc1c(C#N)cccc1-c1cc(-c2cn(Cc3cccc(C(C)(C)O)n3)nn2)nc(N)n1]

In some embodiments, the A_{2A}R antagonist is Preladenant (SCH-420,814), namely 2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine:

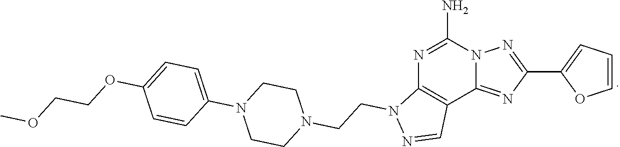 [SMILES: COCCOc1ccc(N2CCN(CCn3ncc4c3nc(N)n3nc(-c5ccco5)nc43)CC2)cc1]

In some embodiments, the A_{2A}R antagonist is Vipadenant (BIIB-014), namely 3-(4-amino-3-methylbenzyl)-7-(2-furyl)-3H-(1,2,3)triazolo(4,5-d)pyrimidine-5-amine:

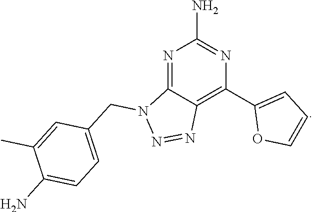 [SMILES: Cc1cc(Cn2nnc3c(-c4ccco4)nc(N)nc32)ccc1N]

In some embodiments, the A_{2A}R antagonist is Tozadenant (SYK-115), namely 4-hydroxy-N-(4-methoxy-7-morpholinobenzo[d]thiazol-2-yl)-4-methylpiperidine-1-carboxamide:

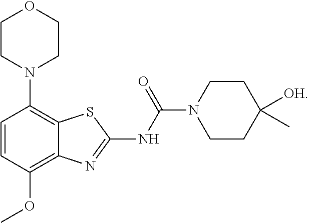 [SMILES: COc1ccc(N2CCOCC2)c2sc(NC(=O)N3CCC(C)(O)CC3)nc12]

In some embodiments, the adenosine receptor antagonist is chosen from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine;
4-hydroxy-N-(4-methoxy-7-morpholinobenzo[d]thiazol-2-yl)-4-methylpiperidine-1-carboxamide;
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and pharmaceutically acceptable salts thereof.

In some embodiments, the adenosine receptor antagonist is 5-bromo-2,6-di-(1H-pyrazol-1-yl)pyrimidin-4-amine. In some embodiments, the adenosine receptor antagonist is (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine. In some embodiments, the adenosine receptor antagonist is 6-(2-chloro-6-methylpyridin-4-yl)-5-(4-fluorophenyl)-1,2,4-triazin-3-amine. In some embodiments, the adenosine receptor antagonist is 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile.

In some embodiments, the adenosine receptor antagonist is chosen from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine;
4-hydroxy-N-(4-methoxy-7-morpholinobenzo[d]thiazol-2-yl)-4-methylpiperidine-1-carboxamide;
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and pharmaceutical salts and solvates thereof.

In one aspect, the present disclosure provides a method or use as described herein, wherein (1) the salt of Compound 1 or hydrate or solvate thereof; the crystalline Compound 1 or hydrate or solvate thereof; the composition as described herein; or the dosage form as described herein is administered prior to, concomitant with, or subsequent to administration of (2) the adenosine receptor antagonist. In one aspect, the present disclosure provides a method or use as described herein, wherein (1) the Compound 1 hydrogen sulfate or hydrate or solvate thereof is administered prior to, concomitant with, or subsequent to administration of (2) the adenosine receptor antagonist.

In some embodiments, the (2) adenosine receptor antagonist is administered prior to the day or on the same day as (1) the salt of Compound 1 or hydrate or solvate thereof; the crystalline Compound 1 or hydrate or solvate thereof; the composition as described herein; or the dosage form as described herein. In some embodiments, the (2) adenosine receptor antagonist is administered prior to the day or on the same day as (1) the Compound 1 hydrogen sulfate or a hydrate or solvate thereof.

In some embodiments, the (2) adenosine receptor antagonist is to be administered prior to and/or concomitantly with (1) the salt of Compound 1 or hydrate or solvate thereof; the crystalline Compound 1 or a hydrate or solvate thereof; the composition as described herein; or the dosage form as described herein, and continuously thereafter. In some embodiments, the (2) adenosine receptor antagonist is to be administered prior to and/or concomitantly with (1) the Compound 1 hydrogen sulfate or a hydrate or solvate thereof, and continuously thereafter.

In some embodiments, (1) the salt of Compound 1 or hydrate or solvate thereof; the crystalline Compound 1 or hydrate or solvate thereof; the composition as described herein; or the dosage form as described herein and (2) the adenosine receptor antagonist may be administered as a single daily dose, divided over one or more daily doses. In some embodiments, (1) the Compound 1 hydrogen sulfate or a hydrate or solvate thereof and (2) the adenosine receptor antagonist may be administered as a single daily dose, divided over one or more daily doses.

In some embodiments, in the methods and uses described herein, the cancer is a solid cancer and non-solid cancer, including benign and malignant solid tumors and benign and malignant non-solid tumors. In some embodiments, the cancer may be metastatic or non-metastatic. In some embodiments, the cancer may be familial or sporadic.

In some embodiments, the cancer is a solid cancer. In some embodiments, the solid tumors are chosen from biliary tract cancer, brain cancer (including glioblastomas and medulloblastomas), breast cancer, carcinoid, cervical cancer, choriocarcinoma, colon cancer, colorectal cancer, endometrial cancer, esophageal cancer, gastric cancer, glioma, head and neck cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumor), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumors (seminomas, and non-seminomas such as teratomas and choriocarcinomas), stromal tumors, germ cell tumors, thyroid cancer (including thyroid adenocarcinoma and medullary carcinoma) and urothelial cancer.

In some embodiments, the cancer is a non-solid cancer. In some embodiments, the non-solid tumors are chosen from hematological neoplasms. As used herein, a hematologic neoplasm is a term of art which includes lymphoid disorders, myeloid disorders, and AIDS associated leukemias. Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease, non-Hodgkin's lymphoma lymphomas, and lymphocytic lymphomas). Chronic lymphoid leukemias include, for example, T cell chronic lymphoid leukemias and B cell chronic lymphoid leukemias.

In some embodiments, the cancer is selected from breast, carcinoid, cervical, colorectal, endometrial, glioma, head and neck, liver, lung, melanoma, ovarian, pancreatic, prostate, renal, gastric, thyroid and urothelial cancers. In some embodiments, the cancer is breast cancer. In some embodiments, the cancer is carcinoid cancer. In some embodiments, the cancer is cervical cancer. In some embodiments, the cancer is colorectal cancer. In some embodiments, the cancer is endometrial cancer. In some embodiments, the cancer is glioma. In some embodiments, the cancer is head and neck cancer. In some embodiments, the cancer is liver cancer. In some embodiments, the cancer is lung cancer. In some embodiments, the cancer is melanoma. In some embodiments, the cancer is ovarian cancer. In some embodiments, the cancer is pancreatic cancer. In some embodiments, the cancer is prostate cancer. In some embodiments, the cancer is renal cancer. In some embodiments, the cancer is gastric cancer. In some embodiments, the cancer is thyroid cancer. In some embodiments, the cancer is urothelial cancer.

In some embodiments, the cancer is chosen from leukemia and multiple myeloma.

In some embodiments, in the methods and uses described herein, the salt or hydrate or solvate thereof is selected from Compound 1 hydrogen chloride, Compound 1 hydrogen sulfate, Compound 1 hydrogen phosphate, Compound 1 tosylate, Compound 1 mesylate, Compound 1 benzenesulfonate, Compound 1 L-tartrate, Compound 1 fumarate, Compound 1 citrate, Compound 1 maleate, Compound 1 L-malate, Compound 1 benzoate, and hydrates and solvates thereof. In some embodiments, in the methods and uses described herein, the salt or hydrate or solvate thereof is not a hexafluorophosphate salt of Compound 1 or hydrate or solvate thereof.

In some embodiments, in the methods and uses described herein, the Compound 1 hydrogen sulfate or hydrate or solvate thereof is chosen from crystalline Form 1 Compound 1 di(hydrogen sulfate), crystalline Form 2 Compound 1 di(hydrogen sulfate) trihydrate, crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), and hydrates and solvates thereof. In some embodiments, in the methods and uses described herein, the Compound 1 hydrogen sulfate or hydrate or solvate thereof is crystalline Form 2 Compound 1 di(hydrogen sulfate) trihydrate.

In some embodiments, in the methods and uses described herein, the Compound 1 hydrogen sulfate or hydrate or solvate thereof is chosen from crystalline Form 4 Compound 1 mono(hydrogen sulfate), crystalline Form 8 Compound 1 mono(hydrogen sulfate), crystalline Form 10 Compound 1 mono(hydrogen sulfate), and hydrates and solvates thereof.

### V. Processes for Preparing Compound 1 Hydrogen Sulfate, Crystalline Compound 1, and Hydrates Thereof

In one aspect, the present disclosure relates a process for preparing Compound 1 hydrogen sulfate or a hydrate or solvate thereof. In some embodiments, the Compound 1 hydrogen sulfate or hydrate or solvate thereof is Compound 1 di(hydrogen sulfate) or a hydrate or solvate thereof. In some embodiments, the process comprises the steps of dissolving Compound 1 (free base) or a hydrate or solvate thereof in a suitable solvent to form a solution, treating the solution with sulfuric acid, and removing the solvent from the solution to form Compound 1 di(hydrogen sulfate) or a hydrate or solvate thereof.

In some embodiments, the suitable solvent is selected from 1,4-dioxane, 1-propanol, 2-Methyl THF, isopropyl alcohol, water, acetone, acetonitrile, DCM, ethanol, methanol, MEK, or combinations thereof. In some embodiments, the suitable solvent is selected from isopropyl alcohol, water, ethoxyethanol, or combinations thereof. In some embodiments, the suitable solvent is selected from isopropyl alcohol, water, or combinations thereof. In some embodiments, the suitable solvent is a combination of isopropyl alcohol and water.

In some embodiments, the solvent is removed by evaporation. In some embodiments, the solvent is removed by anti-solvent addition. In some embodiments, the antisolvent is chosen from tBME, 1-propanol, and a combination thereof.

In some embodiments, the Compound 1 di(hydrogen sulfate) or a hydrate or solvate thereof is crystalline. In some embodiments, the Compound 1 di(hydrogen sulfate) or a hydrate or solvate thereof is crystalline Form 2 Compound 1 di(hydrogen sulfate) trihydrate.

In one aspect, the present disclosure provides Compound 1 di(hydrogen sulfate) or a hydrate or solvate thereof prepared by a process described herein. In some embodiments, the Compound 1 di(hydrogen sulfate) or a hydrate or solvate thereof is crystalline. In some embodiments, the Compound 1 di(hydrogen sulfate) or a hydrate or solvate thereof is crystalline Form 2 Compound 1 di(hydrogen sulfate) trihydrate.

In one aspect, the present disclosure relates a process for preparing Compound 1 mono(hydrogen sulfate) or a hydrate or solvate thereof. In one aspect, the process comprises the steps of dissolving a Compound 1 di(hydrogen sulfate) or a hydrate or solvate thereof in a suitable solvent to form a solution and removing the solvent from the solution to form the Compound 1 mono(hydrogen sulfate) or a hydrate or solvate thereof.

In some embodiments, the solvent is water. In some embodiments, the solvent is removed by filtration. In some embodiments, the solvent is removed by vacuum filtration. In some embodiments, the solvent is water and is removed by filtration. In some embodiments, the solvent is water and is removed by vacuum filtration.

### Enumerated Embodiments

Embodiment 1. Compound 1 hydrogen sulfate or a hydrate or solvate thereof.

Embodiment 2. The Compound 1 hydrogen sulfate or hydrate or solvate thereof of Embodiment 1, wherein the Compound 1 hydrogen sulfate or hydrate or solvate thereof is a mono(hydrogen sulfate) or hydrate or solvate thereof.

Embodiment 3. The Compound 1 hydrogen sulfate or hydrate or solvate thereof of Embodiment 1 or 2, wherein the Compound 1 hydrogen sulfate is a hydrate or solvate.

Embodiment 4. The Compound 1 hydrogen sulfate or hydrate or solvate thereof of any one of Embodiments 1-3, wherein the Compound 1 hydrogen sulfate or hydrate or solvate thereof is crystalline.

Embodiment 5. The Compound 1 hydrogen sulfate or hydrate or solvate thereof of Embodiment 1, wherein the Compound 1 hydrogen sulfate or hydrate or solvate thereof is a di(hydrogen sulfate) or hydrate or solvate thereof.

Embodiment 6. The Compound 1 hydrogen sulfate or hydrate or solvate thereof of Embodiment 5, wherein the Compound 1 hydrogen sulfate or hydrate or solvate thereof is a hydrate.

Embodiment 7. The Compound 1 hydrogen sulfate or hydrate or solvate thereof of any one of Embodiments 1-6, wherein the Compound 1 hydrogen sulfate or hydrate or solvate thereof is a trihydrate.

Embodiment 8. The Compound 1 hydrogen sulfate or hydrate or solvate thereof of any one of Embodiments 5-7, wherein the Compound 1 hydrogen sulfate or hydrate or solvate thereof is crystalline.

Embodiment 9. Crystalline Form 1 Compound 1 di(hydrogen sulfate) or a hydrate or solvate thereof.

Embodiment 10. The Compound 1 hydrogen sulfate or hydrate or solvate thereof or the crystalline Form 1 Compound 1 di(hydrogen sulfate) or a hydrate or solvate thereof of Embodiment 8 or 9, having an X-ray powder diffraction pattern comprising a peak at about 3.7° 2θ.

Embodiment 11. The Compound 1 hydrogen sulfate or hydrate or solvate thereof or the crystalline Form 1 Compound 1 di(hydrogen sulfate) or a hydrate or solvate thereof of Embodiment 8 or 9, having an X-ray powder diffraction pattern comprising one or more peaks chosen from peaks at about 3.7° 2θ, about 7.5° 2θ, about 8.0° 2θ, about 8.8° 2θ, about 11.3° 2θ, about 15.1° 2θ, and about 16.2° 2θ.

Embodiment 12. The Compound 1 hydrogen sulfate or hydrate or solvate thereof or the crystalline Form 1 Compound 1 di(hydrogen sulfate) or a hydrate or solvate thereof of Embodiment 8 or 9, having an X-ray powder diffraction pattern comprising peaks at about 3.7° 2θ, and about 15.1° 2θ.

Embodiment 13. The Compound 1 hydrogen sulfate or hydrate or solvate thereof or the crystalline Form 1 Compound 1 di(hydrogen sulfate) or a hydrate or solvate thereof of any one of Embodiment 8-12, having an X-ray powder diffraction pattern substantially the same as that of FIG. 1.

Embodiment 14. Crystalline Form 2 Compound 1 di(hydrogen sulfate) trihydrate.

Embodiment 15. The Compound 1 hydrogen sulfate or hydrate or solvate thereof or the crystalline Form 2 Compound 1 di(hydrogen sulfate) trihydrate or a hydrate or solvate thereof of Embodiment 8 or 14, having an X-ray powder diffraction pattern comprising a peak at about 4.9° 2θ.

Embodiment 16. The Compound 1 hydrogen sulfate or hydrate or solvate thereof or the crystalline Form 2 Compound 1 di(hydrogen sulfate) trihydrate or a hydrate or solvate thereof of any one of Embodiments 8 or 14-15, having an X-ray powder diffraction pattern comprising peaks at about 4.9° 2θ and about 9.9° 2θ.

Embodiment 17. The Compound 1 hydrogen sulfate or hydrate or solvate thereof or the crystalline Form 2 Compound 1 di(hydrogen sulfate) trihydrate or a hydrate or solvate thereof of any one of Embodiments 8 or 14-15, having an X-ray powder diffraction pattern comprising peaks at about 4.9° 2θ and about 11.7° 2θ.

Embodiment 18. The Compound 1 hydrogen sulfate or hydrate or solvate thereof or the crystalline Form 2 Compound 1 di(hydrogen sulfate) trihydrate or a hydrate or solvate thereof of any one of Embodiments 8 or 14-15, having an X-ray powder diffraction pattern comprising peaks at about 4.9° 2θ and about 15.0° 2θ.

Embodiment 19. The Compound 1 hydrogen sulfate or hydrate or solvate thereof or the crystalline Form 2 Compound 1 di(hydrogen sulfate) trihydrate or a hydrate or solvate thereof of any one of Embodiments 8 or 14-15, having an X-ray powder diffraction pattern comprising peaks at about 4.9° 2θ, about 11.7° 2θ, and about 15.0° 2θ.

Embodiment 20. The Compound 1 hydrogen sulfate or hydrate or solvate thereof or the crystalline Form 2 Compound 1 di(hydrogen sulfate) trihydrate or a hydrate or solvate thereof of any one of Embodiments 8 or 14-16, having an X-ray powder diffraction pattern comprising one or more peaks chosen from about 4.9° 2θ, about 9.9° 2θ about 10.2° 2θ, about 11.7° 2θ, about 12.7° 2θ, about 14.4° 2θ, about 15.0° 2θ, about 15.7° 2θ, about 19.0° 2θ, and about 19.6° 2θ.

Embodiment 21. The Compound 1 hydrogen sulfate or hydrate or solvate thereof or the crystalline Form 2 Compound 1 di(hydrogen sulfate) trihydrate or a hydrate or solvate thereof of any one of Embodiments 8 or 14-16, having an X-ray powder diffraction pattern comprising a peak at about 4.9° 2θ and one or more peaks chosen from peaks at about 10.2° 2θ and about 15.0° 2θ.

Embodiment 22. The Compound 1 hydrogen sulfate or hydrate or solvate thereof or the crystalline Form 2 Compound 1 di(hydrogen sulfate) trihydrate or a hydrate or solvate thereof of any one of Embodiments 8 or 14-21, having an X-ray powder diffraction pattern substantially the same as that of FIG. 3.

Embodiment 23. The Compound 1 hydrogen sulfate or hydrate or solvate thereof or the crystalline Form 2 Compound 1 di(hydrogen sulfate) trihydrate or a hydrate or solvate thereof of any one of Embodiments 8 or 14-22, having an FT-IR spectrum substantially the same as that of FIG. 23A.

Embodiment 24. The Compound 1 hydrogen sulfate or hydrate or solvate thereof or the crystalline Form 2 Compound 1 di(hydrogen sulfate) trihydrate or a hydrate or solvate thereof of any one of Embodiments 8 or 14-23, having a melting onset as measured by DSC in a sealed aluminum pan with a pierced lid of about 144° C.

Embodiment 25. The Compound 1 hydrogen sulfate or hydrate or solvate thereof or the crystalline Form 2 Compound 1 di(hydrogen sulfate) trihydrate or a hydrate or solvate thereof of any one of Embodiments 8 or 14-24, having a DSC thermogram substantially the same as that of FIG. 32.

Embodiment 26. Crystalline Form 3 Compound 1 di(hydrogen sulfate) or a hydrate or solvate thereof.

Embodiment 27. The crystalline Form 3 Compound 1 di(hydrogen sulfate) or hydrate or solvate thereof of Embodiment 26, wherein the Compound 1 di(hydrogen sulfate) is a hydrate or solvate.

Embodiment 28. The crystalline Form 3 Compound 1 di(hydrogen sulfate) or hydrate or solvate thereof of Embodiment 26 or 27, wherein the Compound 1 di(hydrogen sulfate) is a monohydrate.

Embodiment 29. The Compound 1 hydrogen sulfate or hydrate or solvate thereof or crystalline Form 3 Compound 1 di(hydrogen sulfate) or hydrate or solvate thereof of any one of Embodiments 8 or 26-28, having an X-ray powder diffraction pattern comprising a peak at about 3.9° 2θ.

Embodiment 30. The Compound 1 hydrogen sulfate or hydrate or solvate thereof or crystalline Form 3 Compound 1 di(hydrogen sulfate) or hydrate or solvate thereof of any one of Embodiments 8 or 26-28, having an X-ray powder diffraction pattern comprising one or more peaks chosen from peaks at about 3.9° 2θ, about 7.8° 2θ, about 10.2° 2θ, about 15.7° 2θ, and about 19.5° 2θ.

Embodiment 31. The Compound 1 hydrogen sulfate or hydrate or solvate thereof or crystalline Form 3 Compound 1 di(hydrogen sulfate) or hydrate or solvate thereof of any one of Embodiments 8 or 26-28, having an X-ray powder diffraction pattern comprising peaks at about 3.9° 2θ, about 7.8° 2θ, about 10.2° 2θ, about 15.7° 2θ, and about 19.5° 2θ.

Embodiment 32. The Compound 1 hydrogen sulfate or hydrate or solvate thereof or crystalline Form 3 Compound 1 di(hydrogen sulfate) or hydrate or solvate thereof of any one of Embodiments 8 or 26-31, having an X-ray powder diffraction pattern substantially the same as that of FIG. 5.

Embodiment 33. The Compound 1 hydrogen sulfate or hydrate or solvate thereof or crystalline Form 3 Compound 1 di(hydrogen sulfate) or hydrate or solvate thereof of any one of Embodiments 8 or 26-32, having an FT-IR spectrum substantially the same as that of FIG. 23B.

Embodiment 34. The Compound 1 hydrogen sulfate or hydrate or solvate thereof or crystalline Form 3 Compound 1 di(hydrogen sulfate) or hydrate or solvate thereof of any one of Embodiments 8 or 26-33, having a melting onset as measured by DSC in an open aluminum pan of about 163° C.

Embodiment 35. The Compound 1 hydrogen sulfate or hydrate or solvate thereof or crystalline Form 3 Compound 1 di(hydrogen sulfate) or hydrate or solvate thereof of any one of Embodiments 8 or 26-34, having a DSC thermogram substantially the same as that of FIG. 22.

Embodiment 36. Crystalline Form 5 Compound 1 di(hydrogen sulfate) or a hydrate or solvate thereof.

Embodiment 37. The Compound 1 hydrogen sulfate or hydrate or solvate thereof or crystalline Form 5 Compound 1 di(hydrogen sulfate) or hydrate or solvate thereof of Embodiment 8 or 36, having an X-ray powder diffraction pattern comprising a peak at about 5.0° 2θ.

Embodiment 38. The Compound 1 hydrogen sulfate or hydrate or solvate thereof or crystalline Form 5 Compound 1 di(hydrogen sulfate) or hydrate or solvate thereof of Embodiment 8 or 36, having an X-ray powder diffraction pattern comprising one or more peaks chosen from peaks at about 5.0° 2θ, about 7.8° 2θ, about 10.5° 2θ, and about 11.1° 2θ.

Embodiment 39. The Compound 1 hydrogen sulfate or hydrate or solvate thereof or crystalline Form 5 Compound 1 di(hydrogen sulfate) or hydrate or solvate thereof of Embodiment 8 or 38, having an X-ray powder diffraction pattern comprising a peak at about 5.0° 2θ and one or more peaks chosen from peaks at about 7.8° 2θ, about 10.5° 2θ, and about 11.1° 2θ.

Embodiment 40. The Compound 1 hydrogen sulfate or hydrate or solvate thereof or crystalline Form 5 Compound 1 di(hydrogen sulfate) or hydrate or solvate thereof of Embodiment 8 or 36, having an X-ray powder diffraction pattern comprising peaks at about 5.0° 2θ, about 7.8° 2θ, about 10.5° 2θ, and about 11.1° 2θ.

Embodiment 41. The Compound 1 hydrogen sulfate or hydrate or solvate thereof or crystalline Form 5 Compound 1 di(hydrogen sulfate) or hydrate or solvate thereof of any one of Embodiments 8 or 36-40, having an X-ray powder diffraction pattern substantially the same as that of FIG. 11.

Embodiment 42. Crystalline Form 6 Compound 1 di(hydrogen sulfate) or a hydrate or solvate thereof.

Embodiment 43. The Compound 1 hydrogen sulfate or hydrate or solvate thereof or crystalline Form 6 Compound 1 di(hydrogen sulfate) or hydrate or solvate thereof of Embodiment 8 or 42, having an X-ray powder diffraction pattern comprising a peak at about 3.6° 2θ.

Embodiment 44. The Compound 1 hydrogen sulfate or hydrate or solvate thereof or crystalline Form 6 Compound 1 di(hydrogen sulfate) or hydrate or solvate thereof of Embodiment 8 or 42, having an X-ray powder diffraction pattern comprising one or more peaks chosen from peaks at about 3.6° 2θ, about 7.3° 2θ, about 13.1° 2θ, and about 14.6° 2θ.

Embodiment 45. The Compound 1 hydrogen sulfate or hydrate or solvate thereof or crystalline Form 6 Compound 1 di(hydrogen sulfate) or hydrate or solvate thereof of Embodiment 8 or 42, having an X-ray powder diffraction pattern comprising peaks at about 3.6° 2θ, about 7.3° 2θ, about 13.1° 2θ, and about 14.6° 2θ.

Embodiment 46. The Compound 1 hydrogen sulfate or hydrate or solvate thereof or crystalline Form 6 Compound 1 di(hydrogen sulfate) or hydrate or solvate thereof of any one of Embodiments 8 or 42-45, having an X-ray powder diffraction pattern substantially the same as that of FIG. 13.

Embodiment 47. Crystalline Form 7 Compound 1 di(hydrogen sulfate) or a hydrate or solvate thereof.

Embodiment 48. The Compound 1 hydrogen sulfate or hydrate or solvate thereof or crystalline Form 7 Compound 1 di(hydrogen sulfate) or hydrate or solvate thereof of Embodiment 8 or 47, having an X-ray powder diffraction pattern comprising a peak at about 3.6° 2θ.

Embodiment 49. The Compound 1 hydrogen sulfate or hydrate or solvate thereof or crystalline Form 7 Compound 1 di(hydrogen sulfate) or hydrate or solvate thereof of Embodiment 8 or 47, having an X-ray powder diffraction pattern comprising one or more peaks chosen from peaks at about 3.6° 2θ, about 7.1° 2θ, about 9.9° 2θ, and about 12.5° 2θ.

Embodiment 50A. The Compound 1 hydrogen sulfate or hydrate or solvate thereof or crystalline Form 7 Compound 1 di(hydrogen sulfate) or hydrate or solvate thereof of Embodiment 8 or 47, having an X-ray powder diffraction pattern comprising peaks at about 3.6° 2θ, about 7.1° 2θ, about 9.9° 2θ, and about 12.5° 2θ.

Embodiment 50B. The Compound 1 hydrogen sulfate or hydrate or solvate thereof or crystalline Form 7 Compound 1 di(hydrogen sulfate) or hydrate or solvate thereof of Embodiment 8 or 47, having an X-ray powder diffraction pattern comprising peaks at about 3.6° 2θ, about 7.1° 2θ, about 9.9° 2θ, about 12.5° 2θ, and about 21.5° 2θ.

Embodiment 51. The Compound 1 hydrogen sulfate or hydrate or solvate thereof or crystalline Form 7 Compound 1 di(hydrogen sulfate) or hydrate or solvate thereof of any one of Embodiments 8 or 47-50B, having an X-ray powder diffraction pattern substantially the same as that of FIG. 15.

Embodiment 52. Crystalline Form 9 Compound 1 di(hydrogen sulfate) or a hydrate or solvate thereof.

Embodiment 53. The Compound 1 hydrogen sulfate or hydrate or solvate thereof or crystalline Form 9 Compound 1 di(hydrogen sulfate) or hydrate or solvate thereof of Embodiment 8 or 52, having an X-ray powder diffraction pattern comprising a peak at about 4.1° 2θ.

Embodiment 54. The Compound 1 hydrogen sulfate or hydrate or solvate thereof or crystalline Form 9 Compound 1 di(hydrogen sulfate) or hydrate or solvate thereof of Embodiment 8 or 52, having an X-ray powder diffraction pattern comprising one or more peaks chosen from peaks at about 8.3° 2θ, about 10.1° 2θ, about 11.5° 2θ, about 12.3° 2θ, about 13.3° 2θ, and about 16.5° 2θ.

Embodiment 55. The Compound 1 hydrogen sulfate or hydrate or solvate thereof or crystalline Form 9 Compound 1 di(hydrogen sulfate) or hydrate or solvate thereof of Embodiment 8 or 52, having an X-ray powder diffraction pattern comprising peaks at about 8.3° 2θ, about 10.1° 2θ, about 11.5° 2θ, about 12.3° 2θ, about 13.3° 2θ, and about 16.5° 2θ.

Embodiment 56. The Compound 1 hydrogen sulfate or hydrate or solvate thereof or crystalline Form 9 Compound 1 di(hydrogen sulfate) or hydrate or solvate thereof of any one of Embodiments 8 or 52-55, having an X-ray powder diffraction pattern substantially the same as that of FIG. 19.

Embodiment 57. Amorphous Compound 1 di(hydrogen sulfate) or a hydrate or solvate thereof.

Embodiment 58. The amorphous Compound 1 di(hydrogen sulfate) or hydrate or solvate thereof of Embodiment 57, having an X-ray powder diffraction pattern substantially the same as that of FIG. 45.

Embodiment 59. Crystalline Form 4 Compound 1 mono(hydrogen sulfate) or a hydrate or solvate thereof.

Embodiment 60. The crystalline Form 4 Compound 1 mono(hydrogen sulfate) or hydrate or solvate thereof of Embodiment 59, wherein the Compound 1 mono(hydrogen sulfate) or hydrate or solvate thereof is a hydrate.

Embodiment 61. The crystalline Form 4 Compound 1 mono(hydrogen sulfate) or hydrate or solvate thereof of Embodiment 59 or 60, wherein the Compound 1 mono(hydrogen sulfate) or hydrate or solvate thereof is a septahydrate.

Embodiment 62. The Compound 1 hydrogen sulfate or hydrate or solvate thereof or crystalline Form 4 Compound 1 mono(hydrogen sulfate) or hydrate or solvate thereof of any one of Embodiments 4 or 59-61, having an X-ray powder diffraction pattern comprising a peak at about 8.0° 2θ.

Embodiment 63. The Compound 1 hydrogen sulfate or hydrate or solvate thereof or crystalline Form 4 Compound 1 mono(hydrogen sulfate) or hydrate or solvate thereof of any one of Embodiments 4 or 59-61, having an X-ray powder diffraction pattern comprising a peak at about 4.5° 2θ.

Embodiment 64. The Compound 1 hydrogen sulfate or hydrate or solvate thereof or crystalline Form 4 Compound 1 mono(hydrogen sulfate) or hydrate or solvate thereof of any one of Embodiments 4 or 59-61, having an X-ray powder diffraction pattern comprising one or more peaks chosen from peaks at about 4.5° 2θ, about 8.0° 2θ, about 9.0° 2θ, about 9.5° 2θ, about 10.4° 2θ, and about 12.4° 2θ.

Embodiment 65. The Compound 1 hydrogen sulfate or hydrate or solvate thereof or crystalline Form 4 Compound 1 mono(hydrogen sulfate) or hydrate or solvate thereof of any one of Embodiments 4 or 59-61, having an X-ray powder diffraction pattern comprising peaks at about 8.0° 2θ, about 9.0° 2θ, about 10.4° 2θ, and about 12.4° 2θ.

Embodiment 66. The Compound 1 hydrogen sulfate or hydrate or solvate thereof or crystalline Form 4 Compound 1 mono(hydrogen sulfate) or hydrate or solvate thereof of any one of Embodiments 4 or 59-65, having an X-ray powder diffraction pattern substantially the same as that of FIG. 58.

Embodiment 67. The Compound 1 hydrogen sulfate or hydrate or solvate thereof or crystalline Form 4 Compound 1 mono(hydrogen sulfate) or hydrate or solvate thereof of any one of Embodiments 4 or 59-65, having an X-ray powder diffraction pattern substantially the same as that of FIG. 60.

Embodiment 68. The Compound 1 hydrogen sulfate or hydrate or solvate thereof or crystalline Form 4 Compound 1 mono(hydrogen sulfate) or hydrate or solvate thereof of any one of Embodiments 4 or 59-67, having an FT-IR spectrum substantially the same as that of FIG. 63.

Embodiment 69. The Compound 1 hydrogen sulfate or hydrate or solvate thereof or crystalline Form 4 Compound 1 mono(hydrogen sulfate) or hydrate or solvate thereof of any one of Embodiments 4 or 59-68, having a melting onset as measured by DSC in an open aluminum pan of about 148° C.

Embodiment 70. The Compound 1 hydrogen sulfate or hydrate or solvate thereof or crystalline Form 4 Compound 1 mono(hydrogen sulfate) or hydrate or solvate thereof of any one of Embodiments 4 or 59-69, having a DSC thermogram substantially the same as that of FIG. 62.

Embodiment 71. Crystalline Form 8 Compound 1 mono(hydrogen sulfate) or a hydrate or solvate thereof.

Embodiment 72. The crystalline Form 8 Compound 1 mono(hydrogen sulfate) or hydrate or solvate thereof of Embodiment 71, wherein the Compound 1 mono(hydrogen sulfate) or hydrate or solvate thereof is a hydrate.

Embodiment 73. The crystalline Form 8 Compound 1 mono(hydrogen sulfate) or hydrate or solvate thereof of Embodiment 71 or 72, wherein the Compound 1 mono(hydrogen sulfate) or hydrate or solvate thereof is a trihydrate.

Embodiment 74. The Compound 1 hydrogen sulfate or hydrate or solvate thereof or crystalline Form 8 Compound 1 mono(hydrogen sulfate) or hydrate or solvate thereof of any one of Embodiments 4 or 71 or 72, having an X-ray powder diffraction pattern comprising a peak at about 5.1° 2θ.

Embodiment 75. The Compound 1 hydrogen sulfate or hydrate or solvate thereof or crystalline Form 8 Compound 1 mono(hydrogen sulfate) or hydrate or solvate thereof of any one of Embodiments 4 or 71-73, having an X-ray powder diffraction pattern comprising a peak at about 5.1° 2θ and one or more peaks chosen from peaks at about 9.9° 2θ, about 11.2° 2θ, and about 13.8° 2θ.

Embodiment 76. The Compound 1 hydrogen sulfate or hydrate or solvate thereof or crystalline Form 8 Compound 1 mono(hydrogen sulfate) or hydrate or solvate thereof of any one of Embodiments 4 or 71-73, having an X-ray powder diffraction pattern comprising peaks at about 5.1° 2θ, about 9.9° 2θ, about 11.2° 2θ, and about 13.8° 2θ.

Embodiment 77. The Compound 1 hydrogen sulfate or hydrate or solvate thereof or crystalline Form 8 Compound 1 mono(hydrogen sulfate) or hydrate or solvate thereof of any one of Embodiments 4 or 71-76, having an FT-IR spectrum substantially the same as that of FIG. 71.

Embodiment 78. The Compound 1 hydrogen sulfate or hydrate or solvate thereof or crystalline Form 8 Compound 1 mono(hydrogen sulfate) or hydrate or solvate thereof of any one of Embodiments 4 or 71-77, having a melting onset as measured by DSC in an open aluminum pan of about 144° C.

Embodiment 79. The Compound 1 hydrogen sulfate or hydrate or solvate thereof or crystalline Form 8 Compound 1 mono(hydrogen sulfate) or hydrate or solvate thereof of any one of Embodiments 4 or 71-78, having a DSC thermogram substantially the same as that of FIG. 70.

Embodiment 80. Crystalline Form 10 Compound 1 mono(hydrogen sulfate) or a hydrate or solvate thereof.

Embodiment 81. The Compound 1 hydrogen sulfate or hydrate or solvate thereof or the crystalline Form 10 Compound 1 mono(hydrogen sulfate) or hydrate or solvate thereof of Embodiment 4 or 80, having an X-ray powder diffraction pattern comprising a peak at about 11.7° 2θ.

Embodiment 82. The Compound 1 hydrogen sulfate or hydrate or solvate thereof or the crystalline Form 10 Compound 1 mono(hydrogen sulfate) or hydrate or solvate thereof of any one of Embodiments 4 or 80-81, having an X-ray powder diffraction pattern comprising one or more peaks at about 9.6° 2θ, about 10.2° 2θ, about 11.7° 2θ, about 12.4° 2θ, and about 13.1° 2θ.

Embodiment 83. The Compound 1 hydrogen sulfate or hydrate or solvate thereof or the crystalline Form 10 Compound 1 mono(hydrogen sulfate) or hydrate or solvate thereof of any one of Embodiments 4 or 80-81, having an X-ray powder diffraction pattern comprising peaks at about 9.6° 2θ, about 10.2° 2θ, about 11.7° 2θ, about 12.4° 2θ, and about 13.1° 2θ.

Embodiment 84. The Compound 1 hydrogen sulfate or hydrate or solvate thereof or the crystalline Form 10 Compound 1 mono(hydrogen sulfate) or hydrate or solvate thereof of any one of Embodiments 4 or 80-83, having an X-ray powder diffraction pattern substantially the same as that of FIG. 54.

Embodiment 85. Amorphous Compound 1 mono(hydrogen sulfate) or a hydrate or solvate thereof.

Embodiment 86. The amorphous Compound 1 mono(hydrogen sulfate) or hydrate or solvate thereof according to Embodiment 85, having an X-ray powder diffraction pattern substantially the same as that of FIG. 56.

Embodiment 87. Crystalline Compound 1 or a hydrate or solvate thereof.

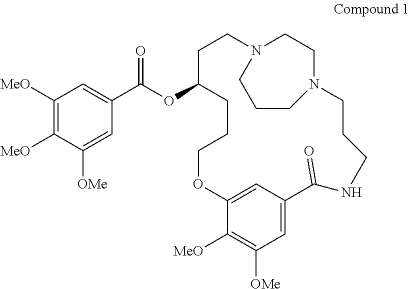 [SMILES: COc1cc(C(=O)O[C@@H]2CCCOc3cc(cc(OC)c3OC)C(=O)NCCCN3CCCN(CC2)CC3)cc(OC)c1OC]

Embodiment 88. Crystalline Form 1 Compound 1 or a hydrate or solvate thereof.

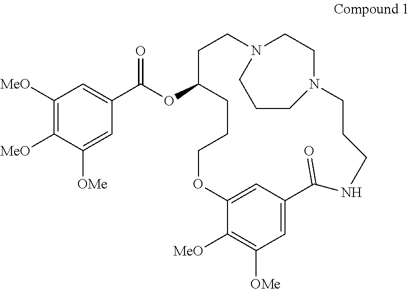 [SMILES: COc1cc(C(=O)O[C@@H]2CCCOc3cc(cc(OC)c3OC)C(=O)NCCCN3CCCN(CC2)CC3)cc(OC)c1OC]

Embodiment 89. The crystalline Compound 1 or hydrate or solvate thereof or crystalline Form 1 Compound 1 or hydrate or solvate thereof of Embodiment 87 or 88, having an X-ray powder diffraction pattern substantially the same as that of FIG. 51.

Embodiment 90. Crystalline Form 2 Compound 1 or a hydrate or solvate thereof.

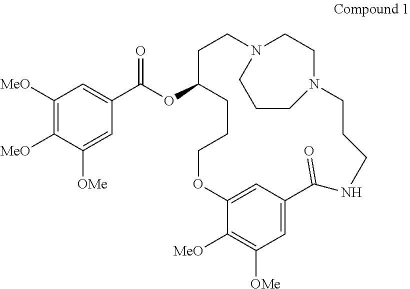 [SMILES: COc1cc(C(=O)O[C@@H]2CCCOc3cc(cc(OC)c3OC)C(=O)NCCCN3CCCN(CC2)CC3)cc(OC)c1OC]

Embodiment 91. The crystalline Compound 1 or hydrate or solvate thereof or crystalline Form 2 Compound 1 or hydrate or solvate thereof of Embodiment 87 or 90, having an X-ray powder diffraction pattern substantially the same as that of FIG. 52.

Embodiment 92. Substantially crystalline Compound 1 di(hydrogen sulfate) or a hydrate or solvate thereof.

Embodiment 93. The substantially crystalline Compound 1 di(hydrogen sulfate) or hydrate or solvate thereof according to Embodiment 92, wherein the substantially crystalline Compound 1 di(hydrogen sulfate) or hydrate or solvate thereof is more than about 50% crystalline.

Embodiment 94. A composition comprising two or more of crystalline Form 1 Compound 1 di(hydrogen sulfate), crystalline Form 2 Compound 1 di(hydrogen sulfate) trihydrate, crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), and hydrates and solvates thereof.

Embodiment 95. The composition of Embodiment 94, wherein at least one of the crystalline Form 1 Compound 1 di(hydrogen sulfate), crystalline Form 2 Compound 1 di(hydrogen sulfate) trihydrate, crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), or hydrates or solvates thereof is substantially crystalline.

Embodiment 96. The composition of Embodiment 95, wherein at least one of the crystalline Form 1 Compound 1 di(hydrogen sulfate), crystalline Form 2 Compound 1 di(hydrogen sulfate) trihydrate, crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), or hydrates or solvates thereof is more than about 50% crystalline.

Embodiment 97. Substantially crystalline Form 2 Compound 1 di(hydrogen sulfate) trihydrate.

Embodiment 98. The substantially crystalline Form 2 Compound 1 di(hydrogen sulfate) trihydrate according to Embodiment 97, wherein the substantially crystalline Form 2 Compound 1 di(hydrogen sulfate) trihydrate is more than about 50% crystalline.

Embodiment 99. A composition comprising substantially crystalline Form 2 Compound 1 di(hydrogen sulfate) trihydrate, further comprising one or more of crystalline Form 1 Compound 1 di(hydrogen sulfate), crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), and hydrates and solvates thereof.

Embodiment 100. The composition according to Embodiment 99, wherein the substantially crystalline Form 2 Compound 1 di(hydrogen sulfate) trihydrate is more than about 50% crystalline.

Embodiment 101. A composition comprising amorphous Compound 1 di(hydrogen sulfate) or a hydrate or solvate thereof and one or more of crystalline Form 1 Compound 1 di(hydrogen sulfate), crystalline Form 2 Compound 1 di(hydrogen sulfate) trihydrate, crystalline Form 3 Compound 1 di(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 9 Compound 1 di(hydrogen sulfate), and hydrates and solvates thereof.

Embodiment 102. A composition comprising amorphous Compound 1 mono(hydrogen sulfate) or a hydrate or solvate thereof and one or more of crystalline Form 4 Compound 1 mono(hydrogen sulfate), crystalline Form 8 Compound 1 mono(hydrogen sulfate), crystalline Form 10 mono(hydrogen sulfate), and hydrates and solvates thereof.

Embodiment 103. A pharmaceutical composition comprising:
the Compound 1 hydrogen sulfate or hydrate or solvate thereof according to any one of Embodiment 1-86, 92-93, or 97-98 or the composition according to any one of Embodiments 94-96 and 99-102,
and at least one pharmaceutically acceptable excipient.

Embodiment 104. The pharmaceutical composition of Embodiment 103, wherein the Compound 1 hydrogen sulfate or hydrate or solvate thereof is the Compound 1 hydrogen sulfate or hydrate or solvate thereof of any one of Embodiments 4, 8-56, 59-84, 92-93 or 97-98.

Embodiment 105. The pharmaceutical composition of Embodiment 103, wherein the Compound 1 hydrogen sulfate or hydrate or solvate thereof is the Compound 1 hydrogen sulfate or hydrate or solvate thereof of any one of Embodiments 57-58 or 85-86.

Embodiment 106. A pharmaceutical composition comprising crystalline Compound 1 or hydrate or solvate thereof according to any one of Embodiments 87-91 and at least one pharmaceutically acceptable excipient.

Embodiment 107. A dosage form comprising a pharmaceutical composition of any one of Embodiments 103-106.

Embodiment 108. The dosage form of Embodiment 107, wherein the dosage form is a solid dosage form.

Embodiment 109. The dosage form of Embodiment 107 or 108, wherein the dosage form is an oral dosage form.

Embodiment 110. A method of inhibiting ENT1 in a patient need thereof, comprising: administering to said patient an effective amount of a Compound 1 hydrogen sulfate or hydrate or solvate thereof according to any one of Embodiments 1-86, 92, 93, or 97-98; an effective amount of crystalline Compound 1 or hydrate or solvate thereof according to any one of Embodiments 87-91; a composition or pharmaceutical composition according to any one of Embodiments 94-96 or 99-106; or a dosage form according to any one of Embodiments 107-109.

Embodiment 111. Use of Compound 1 hydrogen sulfate or hydrate or solvate thereof according to any one of Embodiments 1-86, 92, 93, or 97-98; crystalline Compound 1 or hydrate or solvate thereof according to any one of Embodiments 87-91; composition or pharmaceutical composition according to any one of Embodiments 94-96 or 99-106; or dosage form according to any one of Embodiments 107-109, for the manufacture of a medicament for inhibiting ENT1.

Embodiment 112. Compound 1 hydrogen sulfate or hydrate or solvate thereof according to any one of Embodiments 1-86, 92, 93, or 97-98; crystalline Compound 1 or hydrate or solvate thereof according to any one of Embodiments 87-91; composition or pharmaceutical composition according to any one of Embodiments 94-96 or 99-106; or dosage form according to any one of Embodiments 107-109 for use as a medicament.

Embodiment 113. A method of treating cancer in a patient need thereof, comprising: administering to said patient an effective amount of a Compound 1 hydrogen sulfate or hydrate or solvate thereof according to any one of Embodiments 1-86, 92, 93, or 97-98; an effective amount of crystalline Compound 1 or hydrate or solvate thereof according to any one of Embodiments 87-91; a composition or pharmaceutical composition according to any one of Embodiments 94-96 or 99-106; or a dosage form according to any one of Embodiments 107-109.

Embodiment 114. Use of Compound 1 hydrogen sulfate or hydrate or solvate thereof according to any one of Embodiments 1-86, 92, 93, or 97-98; crystalline Compound 1 or hydrate or solvate thereof according to any one of Embodiments 87-91; composition or pharmaceutical composition according to any one of Embodiments 94-96 or 99-106; or dosage form according to any one of Embodiments 107-109, for the manufacture of a medicament for treating cancer.

Embodiment 115. Compound 1 hydrogen sulfate or hydrate or solvate thereof according to any one of Embodiments 1-86, 92, 93, or 97-98; crystalline Compound 1 or hydrate or solvate thereof according to any one of Embodiments 87-91; composition or pharmaceutical composition according to any one of Embodiments 94-96 or 99-106; or dosage form according to any one of Embodiments 107-109, for use in a method of treating cancer.

Embodiment 116. A method of treating cancer in a patient need thereof, comprising: administering to said patient (1) an effective amount of Compound 1 hydrogen sulfate or hydrate or solvate thereof according to any one of Embodiments 1-86, 92, 93, or 97-98; crystalline Compound 1 or hydrate or solvate thereof according to any one of Embodiments 87-91; composition or pharmaceutical composition according to any one of Embodiments 94-96 or 99-106; or dosage form according to any one of Embodiments 107-109; and (2) an adenosine receptor antagonist.

Embodiment 117. Use of a combination of (1) Compound 1 hydrogen sulfate or hydrate or solvate thereof according to any one of Embodiments 1-86, 92, 93, or 97-98; crystalline Compound 1 or hydrate or solvate thereof according to any one of Embodiments 87-91; composition or pharmaceutical composition according to any one of Embodiments 94-96 or 99-106; or dosage form according to any one of Embodiments 107-109; and (2) an adenosine receptor antagonist for the manufacture of a medicament for treating cancer.

Embodiment 118. The method or use of Embodiment 115 or 116, wherein (1) the Compound 1 hydrogen sulfate or hydrate or solvate thereof according to any one of Embodiments 1-86, 92, 93, or 97-98; crystalline Compound 1 or hydrate or solvate thereof according to any one of Embodiments 87-91; composition or pharmaceutical composition according to any one of Embodiments 94-96 or 99-106; or dosage form according to any one of Embodiments 107-109 is administered prior to, concomitant with, or subsequent to administration of (2) the adenosine receptor antagonist.

Embodiment 119. The method or use of any one of Embodiments 116-118, wherein the adenosine receptor antagonist is an A2A or A2B receptor antagonist.

Embodiment 120A. The method or use of any one of Embodiments 116-119, wherein the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine;
4-hydroxy-N-(4-methoxy-7-morpholinobenzo[d]thiazol-2-yl)-4-methylpiperidine-1-carboxamide;
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
   and pharmaceutical salts and solvates thereof.

Embodiment 120B. The method or use of any one of Embodiments 116-119, wherein the adenosine receptor antagonist is a hydrochloride salt of (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one.

Embodiment 120C. The method or use of Embodiment 120B, wherein the adenosine receptor antagonist is the hydrochloride salt of (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one is Form 2.

Embodiment 121. A process for preparing Compound 1 di(hydrogen sulfate) or a hydrate or solvate thereof comprising the steps of dissolving Compound 1 or a hydrate or solvate thereof in a suitable solvent to form a solution, treating the solution with sulfuric acid, and removing the solvent from the solution to form the Compound 1 di(hydrogen sulfate) or hydrate or solvate thereof.

Embodiment 122. The process of Embodiment 121, wherein the suitable solvent is selected from isopropyl alcohol, water, ethoxyethanol, or combinations thereof.

Embodiment 123. The process of Embodiment 121 or 122, wherein the solvent is removed by evaporation.

Embodiment 124. The process of Embodiment 121 or 122, wherein the solvent is removed by anti-solvent addition.

Embodiment 125. The process of Embodiment 124, wherein the antisolvent is chosen from tBME, 1-propanol, and a combination thereof.

Embodiment 126. The process according to any one of Embodiments 121-125, wherein the Compound 1 di(hydrogen sulfate) or hydrate or solvate thereof is crystalline.

Embodiment 127. Compound 1 di(hydrogen sulfate) or a hydrate or solvate thereof prepared by the processes of any one of Embodiments 121-125.

Embodiment 128. The Compound 1 di(hydrogen sulfate) or hydrate or solvate thereof according to Embodiment 127, wherein the Compound 1 di(hydrogen sulfate) or hydrate or solvate thereof is crystalline.

Embodiment 129. A process for preparing Compound 1 mono(hydrogen sulfate) or a hydrate or solvate thereof comprising the steps of dissolving a Compound 1 di(hydrogen sulfate) or a hydrate or solvate thereof in a suitable solvent to form a solution and removing the solvent from the solution to form the Compound 1 mono(hydrogen sulfate) or a hydrate or solvate thereof.

Embodiment 130. The process of Embodiment 129, wherein the solvent is water and is removed by vacuum filtration.

Embodiment 131. The process according to Embodiment 129 or 130, wherein the Compound 1 mono(hydrogen sulfate) or a hydrate or solvate thereof is crystalline.

Embodiment 132. Compound 1 mono(hydrogen sulfate) or a hydrate or solvate thereof prepared by the process of Embodiment 129 or 130.

Embodiment 133. The Compound 1 mono(hydrogen sulfate) or hydrate or solvate thereof according to Embodiment 132, wherein the Compound 1 mono(hydrogen sulfate) or a hydrate or solvate thereof is crystalline.

Embodiment 134. A salt of Compound 1 or a hydrate or solvate thereof, wherein the salt or hydrate or solvate thereof is not a hexafluorophosphate salt of Compound 1 or hydrate or solvate thereof.

Embodiment 135. A pharmaceutically acceptable salt of Compound 1 or a hydrate or solvate thereof.

Embodiment 136. A crystalline complex comprising Compound 1 and a coformer, or a hydrate or solvate of the crystalline complex.

Embodiment 137. A crystalline salt of Compound 1 comprising Compound 1 and a salt coformer, or a hydrate or solvate of the crystalline salt.

Embodiment 138. The crystalline salt or hydrate or solvate thereof of Embodiment 137, wherein the salt coformer is an inorganic acid.

Embodiment 139. The crystalline salt or hydrate or solvate thereof of Embodiment 138, wherein the salt coformer is hydrochloric acid, sulfuric acid, or phosphoric acid.

Embodiment 140A. The crystalline salt or hydrate or solvate thereof of Embodiment 139, wherein the salt coformer is hydrochloric acid.

Embodiment 140B. The crystalline salt or hydrate or solvate thereof of Embodiment 139, wherein the salt coformer is sulfuric acid.

Embodiment 140C. The crystalline salt or hydrate or solvate thereof of Embodiment 139, wherein the salt coformer is phosphoric acid.

Embodiment 141. The crystalline salt or hydrate or solvate thereof of Embodiment 137, wherein the salt coformer is an organic acid.

Embodiment 142. The crystalline salt or hydrate or solvate thereof of Embodiment 141, wherein the organic acid is an aromatic acid.

Embodiment 143. The crystalline salt or hydrate or solvate thereof of Embodiment 142, wherein the aromatic acid is benzoic acid.

Embodiment 144. The crystalline salt or hydrate or solvate thereof of Embodiment 141, wherein the organic acid is a di-acid.

Embodiment 145. The crystalline salt or hydrate or solvate thereof of Embodiment 144, wherein the di-acid contains at least one hydroxyl group.

Embodiment 146. The crystalline salt or hydrate or solvate thereof of Embodiment 144, wherein the di-acid is L-malic acid.

Embodiment 147. The crystalline salt or hydrate or solvate thereof of Embodiment 144, wherein the di-acid is fumaric acid.

Embodiment 148. The crystalline salt or hydrate or solvate thereof of any one of Embodiments 137-147, wherein the salt or hydrate or solvate thereof is a Compound 1 mono(salt coformer) salt or hydrate or solvate thereof.

Embodiment 149. The crystalline salt or hydrate or solvate thereof of any one of Embodiments 137-147, wherein the salt or hydrate or solvate thereof is a Compound 1 di(salt coformer) salt or hydrate or solvate thereof.

Embodiment 150. Amorphous Compound 1 hydrogen chloride or a hydrate or solvate thereof.

Embodiment 151. Amorphous Compound 1 mono(hydrogen chloride) or a hydrate or solvate thereof.

Embodiment 152. Amorphous Compound 1 di(hydrogen chloride) or a hydrate or solvate thereof.

Embodiment 153. Crystalline Compound 1 hydrogen chloride or a hydrate or solvate thereof.

Embodiment 154. Crystalline Compound 1 di(hydrogen chloride) or a hydrate or solvate thereof.

Embodiment 155. Crystalline Form 1 Compound 1 hydrogen chloride or a hydrate or solvate thereof.

Embodiment 156. The crystalline Form 1 Compound 1 hydrogen chloride or hydrate or solvate thereof of Embodiment 155, wherein the crystalline Form 1 Compound 1 hydrogen chloride or hydrate or solvate thereof is crystalline Form 1 Compound 1 di(hydrogen chloride) or hydrate or solvate thereof.

Embodiment 157. The crystalline Compound 1 hydrogen chloride or hydrate or solvate thereof or the crystalline Compound 1 di(hydrogen chloride) or hydrate or solvate thereof of any one of Embodiments 140A, 149, or 153-156, wherein the Compound 1 hydrogen chloride or hydrate or solvate thereof or the Compound 1 di(hydrogen chloride) or hydrate or solvate thereof is a hydrate.

Embodiment 158. The crystalline Compound 1 hydrogen chloride or hydrate or solvate thereof or the crystalline Compound 1 di(hydrogen chloride) or hydrate or solvate thereof of any one of Embodiments 140A, 149, or 153-157, having an X-ray powder diffraction pattern comprising a peak at about 8.5° 2θ.

Embodiment 159. The crystalline Compound 1 hydrogen chloride or hydrate or solvate thereof or the crystalline Compound 1 di(hydrogen chloride) or hydrate or solvate thereof of any one of Embodiments 140A, 149, or 153-157, having an X-ray powder diffraction pattern comprising peaks at about 8.5° 2θ, about 9.6° 2θ, about 13.2° 2θ, about 14.0° 2θ, and about 15.7° 2θ.

Embodiment 160. The crystalline Compound 1 hydrogen chloride or hydrate or solvate thereof or the crystalline Compound 1 di(hydrogen chloride) or hydrate or solvate thereof of any one of Embodiments 140A, 149, or 153-157, having an X-ray powder diffraction pattern comprising one or more peaks chosen from about 8.5° 2θ, about 9.6° 2θ, about 13.2° 2θ, about 14.0° 2θ, and about 15.7° 2θ.

Embodiment 161. The crystalline Compound 1 hydrogen chloride or hydrate or solvate thereof or the crystalline Compound 1 di(hydrogen chloride) or hydrate or solvate thereof of any one of Embodiments 140A, 149, or 153-160, having an X-ray powder diffraction pattern substantially the same as that of FIG. 103.

Embodiment 162. The crystalline Compound 1 hydrogen chloride or hydrate or solvate thereof or the crystalline Compound 1 di(hydrogen chloride) or hydrate or solvate thereof of any one of Embodiments 140A, 149, or 153-160, having a DSC endotherm with an onset of about 163° C.

Embodiment 163. The crystalline Compound 1 hydrogen chloride or hydrate or solvate thereof or the crystalline Compound 1 di(hydrogen chloride) or hydrate or solvate thereof of Embodiment 162, wherein the endotherm is a melting endotherm.

Embodiment 164. The crystalline Compound 1 hydrogen chloride or hydrate or solvate thereof or the crystalline Compound 1 di(hydrogen chloride) or hydrate or solvate thereof of any one of Embodiments 140A, 149, or 153-163, having a DSC thermogram substantially the same as that of FIG. 105A or FIG. 105B.

Embodiment 165. Crystalline Compound 1 hydrogen phosphate or a hydrate or solvate thereof.

Embodiment 166. Crystalline Compound 1 mono(hydrogen phosphate) or a hydrate or solvate thereof.

Embodiment 167. Crystalline Form 1 Compound 1 hydrogen phosphate or a hydrate or solvate thereof.

Embodiment 168. The crystalline Form 1 Compound 1 hydrogen phosphate or hydrate or solvate thereof of Embodiment 167, wherein the crystalline Form 1 Compound 1 hydrogen phosphate or hydrate or solvate thereof is crystalline Form 1 Compound 1 mono(hydrogen phosphate) or hydrate or solvate thereof.

Embodiment 169. The crystalline Compound 1 hydrogen phosphate or hydrate or solvate thereof or the crystalline Compound 1 mono(hydrogen phosphate) or hydrate or solvate thereof of any one of Embodiments 140C, 148, or 165-168, having an X-ray powder diffraction pattern comprising a diffraction pattern substantially the same as that of FIG. 109.

Embodiment 170. Crystalline Form 2 Compound 1 hydrogen phosphate or hydrate or solvate thereof.

Embodiment 171. The crystalline Form 2 Compound 1 hydrogen phosphate or hydrate or solvate thereof of Embodiment 170, wherein the crystalline Form 2 Compound 1 hydrogen phosphate or hydrate or solvate thereof is crystalline Form 2 Compound 1 mono(hydrogen phosphate) or hydrate or solvate thereof.

Embodiment 172. The crystalline Compound 1 hydrogen phosphate or hydrate or solvate thereof or the crystalline Compound 1 mono(hydrogen phosphate) or hydrate or solvate thereof of any one of Embodiments 140C, 148, 165-166, or 170-171, having an X-ray powder diffraction pattern comprising a diffraction pattern substantially the same as that of FIG. 113.

Embodiment 173. Crystalline Form 3 Compound 1 hydrogen phosphate or hydrate or solvate thereof.

Embodiment 174. The crystalline Form 3 Compound 1 hydrogen phosphate or hydrate or solvate thereof of Embodiment 173, wherein the crystalline Form 3 Compound 1 hydrogen phosphate or hydrate or solvate thereof is crystalline Form 3 Compound 1 mono(hydrogen phosphate) or hydrate or solvate thereof.

Embodiment 175. The crystalline Compound 1 hydrogen phosphate or hydrate or solvate thereof or the crystalline Compound 1 mono(hydrogen phosphate) or hydrate or solvate thereof of any one of Embodiments 140C, 148, 165-166, or 173-174, having an X-ray powder diffraction pattern comprising a diffraction pattern substantially the same as that of FIG. 117.

Embodiment 176. Crystalline Form 4 Compound 1 hydrogen phosphate or hydrate or solvate thereof.

Embodiment 177. The crystalline Form 4 Compound 1 hydrogen phosphate or hydrate or solvate thereof of Embodiment 176, wherein the crystalline Form 4 Compound 1 hydrogen phosphate or hydrate or solvate thereof is crystalline Form 4 Compound 1 mono(hydrogen phosphate) or hydrate or solvate thereof.

Embodiment 178. The crystalline Compound 1 hydrogen phosphate or hydrate or solvate thereof or the crystalline Compound 1 mono(hydrogen phosphate) or hydrate or solvate thereof of any one of Embodiments 140C, 148, 165-166, or 176-178, having an X-ray powder diffraction pattern comprising a diffraction pattern substantially the same as that of FIG. 119.

Embodiment 179. The crystalline Compound 1 hydrogen phosphate or hydrate or solvate thereof or the crystalline Compound 1 mono(hydrogen phosphate) or hydrate or solvate thereof of any one of Embodiments 140C, 148, or 165-178, having an X-ray powder diffraction pattern comprising a peak at about 3.7° 2θ.

Embodiment 180. Crystalline Compound 1 di(hydrogen phosphate) or hydrate or solvate thereof.

Embodiment 181. Crystalline Form 5 Compound 1 hydrogen phosphate or hydrate or solvate thereof.

Embodiment 182. The crystalline Form 5 Compound 1 hydrogen phosphate or hydrate or solvate thereof of Embodiment 181, wherein the crystalline Form 5 Compound 1 hydrogen phosphate or hydrate or solvate thereof is crystalline Form 5 Compound 1 di(hydrogen phosphate) or hydrate or solvate thereof.

Embodiment 183. The crystalline Compound 1 hydrogen phosphate or hydrate or solvate thereof or the crystalline Compound 1 di(hydrogen phosphate) or hydrate or solvate thereof of any one of Embodiments 140C, 149, 165, or 180-182, having an X-ray powder diffraction pattern comprising a peak at about 4.0° 2θ.

Embodiment 184. The crystalline Compound 1 hydrogen phosphate or hydrate or solvate thereof or the crystalline Compound 1 di(hydrogen phosphate) or hydrate or solvate thereof of any one of Embodiments 140C, 149, 165, or 180-182, having an X-ray powder diffraction pattern comprising peaks at about 4.0° 2θ, about 8.2° 2θ, and about 9.7° 2θ.

Embodiment 185. The crystalline Compound 1 hydrogen phosphate or hydrate or solvate thereof or the crystalline Compound 1 di(hydrogen phosphate) or hydrate or solvate thereof of any one of Embodiments 140C, 149, 165, or 180-182, having an X-ray powder diffraction pattern substantially the same as that of FIG. 121.

Embodiment 186. Crystalline Form 6 Compound 1 hydrogen phosphate or hydrate or solvate thereof.

Embodiment 187. The crystalline Form 6 Compound 1 hydrogen phosphate or hydrate or solvate thereof of Embodiment 186, wherein the crystalline Form 6 Compound 1 hydrogen phosphate or hydrate or solvate thereof is crystalline Form 6 Compound 1 di(hydrogen phosphate) or hydrate or solvate thereof.

Embodiment 188. The crystalline Compound 1 hydrogen phosphate or hydrate or solvate thereof or the crystalline Compound 1 di(hydrogen phosphate) or hydrate or solvate thereof of any one of Embodiments 140C, 149, 165, or 186-187, having an X-ray powder diffraction pattern comprising a peak at about 3.1° 2θ.

Embodiment 189. The crystalline Compound 1 hydrogen phosphate or hydrate or solvate thereof or the crystalline Compound 1 di(hydrogen phosphate) or hydrate or solvate thereof of any one of Embodiments 140C, 149, 165, 180, or 186-187, having an X-ray powder diffraction pattern comprising peaks at about 3.1° 2θ and about 5.6° 2θ.

Embodiment 190. The crystalline Compound 1 hydrogen phosphate or hydrate or solvate thereof or the crystalline Compound 1 di(hydrogen phosphate) or hydrate or solvate thereof of any one of Embodiments 140C, 149, 165, 180, or 186-189, having an X-ray powder diffraction pattern substantially the same as that of FIG. 123.

Embodiment 191. The crystalline Compound 1 hydrogen phosphate or hydrate or solvate thereof or the crystalline Compound 1 di(hydrogen phosphate) or hydrate or solvate thereof of any one of Embodiments 140C, 149, 165, 180, or 186-190, having a DSC endotherm with an onset of about 141° C.

Embodiment 192. The crystalline Compound 1 hydrogen phosphate or hydrate or solvate thereof or the crystalline Compound 1 di(hydrogen phosphate) or hydrate or solvate thereof of Embodiment 191, wherein the endotherm is a melting endotherm.

Embodiment 193. The crystalline Compound 1 hydrogen phosphate or hydrate or solvate thereof or the crystalline Compound 1 di(hydrogen phosphate) or hydrate or solvate thereof of any one of Embodiments 140C, 149, 165, 180, or 186-192, having a DSC thermogram substantially the same as that of FIG. 128.

Embodiment 194. Crystalline Form 8 Compound 1 hydrogen phosphate or hydrate or solvate thereof.

Embodiment 195. The crystalline Form 8 Compound 1 hydrogen phosphate or hydrate or solvate thereof of Embodiment 194, wherein the crystalline Form 8 Compound 1 hydrogen phosphate or hydrate or solvate thereof is crystalline Form 8 Compound 1 di(hydrogen phosphate) or hydrate or solvate thereof.

Embodiment 196. The crystalline Compound 1 hydrogen phosphate or hydrate or solvate thereof or the crystalline Compound 1 di(hydrogen phosphate) or hydrate or solvate thereof of any one of Embodiments 140C, 149, 165, 180, or 194-195, having an X-ray powder diffraction pattern comprising a peak at about 3.8° 2θ.

Embodiment 197. The crystalline Compound 1 hydrogen phosphate or hydrate or solvate thereof or the crystalline Compound 1 di(hydrogen phosphate) or hydrate or solvate thereof of any one of Embodiments 140C, 149, 165, 180, or 194-195, having an X-ray powder diffraction pattern comprising one or more peaks at about 3.8° 2θ, and about 16.2° 2θ.

Embodiment 198. The crystalline Compound 1 hydrogen phosphate or hydrate or solvate thereof or the crystalline Compound 1 di(hydrogen phosphate) or hydrate or solvate thereof of any one of Embodiments 140C, 149, 165, 180, or 194-197, having an X-ray powder diffraction pattern substantially the same as that of FIG. 125.

Embodiment 199. The crystalline Compound 1 hydrogen phosphate or hydrate or solvate thereof or the crystalline Compound 1 di(hydrogen phosphate) or hydrate or solvate thereof of any one of Embodiments 140C, 149, 165, or 180-197, having an X-ray powder diffraction pattern comprising peak at about 4.9° 2θ.

Embodiment 200. An amorphous salt of Compound 1 comprising Compound 1 and a salt coformer, or a hydrate or solvate of said amorphous salt.

Embodiment 201. An amorphous salt of Compound 1 comprising Compound 1 and a salt coformer, or a hydrate or solvate of said amorphous salt, wherein the amorphous salt or hydrate or solvate thereof is not a hexafluorophosphate salt of Compound 1 or a hydrate or solvate thereof.

Embodiment 202. The amorphous salt of Compound 1 or hydrate or solvate thereof of any one of Embodiments 200 or 201, wherein the salt coformer is selected from hydrochloric acid, sulfuric acid, p-toluenesulfonic acid, phosphoric acid, methanesulfonic acid, benzenesulfonic acid, L-tartaric acid, fumaric acid, citric acid, maleic acid, L-malic acid, and benzoic acid.

Embodiment 203. The amorphous salt of Compound 1 or hydrate or solvate thereof of any one of Embodiments 200-202, wherein the amorphous salt or hydrate or solvate thereof is a mono(salt coformer) salt or hydrate or solvate thereof.

Embodiment 204. The amorphous salt of Compound 1 or hydrate or solvate thereof of any one of Embodiments 200-202, wherein the amorphous salt or hydrate or solvate thereof is a di(salt coformer) salt or hydrate or solvate thereof.

Embodiment 205. The amorphous salt of Compound 1 or hydrate or solvate thereof of any one of Embodiments 202-204, wherein the salt coformer is selected from p-toluenesulfonic acid, methanesulfonic acid, and benzenesulfonic acid.

Embodiment 206. Crystalline Compound 1 fumarate or a hydrate or solvate thereof.

Embodiment 207. Crystalline Compound 1 mono(fumarate) or a hydrate or solvate thereof.

Embodiment 208. Crystalline Form 1 Compound 1 fumarate or a hydrate or solvate thereof.

Embodiment 209. The crystalline Form 1 Compound 1 fumarate or hydrate or solvate thereof of Embodiment 208, wherein the crystalline Form 1 Compound 1 fumarate or hydrate or solvate thereof is crystalline Form 1 Compound 1 mono(fumarate) or hydrate or solvate thereof.

Embodiment 210. The crystalline Compound 1 fumarate or hydrate or solvate thereof or the crystalline Compound 1 mono(fumarate) or hydrate or solvate thereof of any one of Embodiments 147 or 206-209, having an X-ray powder diffraction pattern comprising a peak at about 8.4° 2θ.

Embodiment 211. The crystalline Compound 1 fumarate or hydrate or solvate thereof or the crystalline Compound 1 mono(fumarate) or hydrate or solvate thereof of any one of Embodiments 147 or 206-209, having an X-ray powder diffraction pattern comprising peaks at about 8.4° 2θ and about 14.5° 2θ.

Embodiment 212. The crystalline Compound 1 fumarate or hydrate or solvate thereof or the crystalline Compound 1 mono(fumarate) or hydrate or solvate thereof of any one of Embodiments 147 or 206-211, having an X-ray powder diffraction pattern substantially the same as that of FIG. 133.

Embodiment 213. Crystalline Form 2 Compound 1 fumarate or hydrate or solvate thereof.

Embodiment 214. The crystalline Form 2 Compound 1 fumarate or hydrate or solvate thereof of Embodiment 213, wherein the crystalline Form 2 Compound 1 fumarate or hydrate or solvate thereof is crystalline Form 2 Compound 1 mono(fumarate) or hydrate or solvate thereof.

Embodiment 215. The crystalline Compound 1 fumarate or hydrate or solvate thereof or the crystalline Compound 1 mono(fumarate) or hydrate or solvate thereof of any one of Embodiments 147, 206-207, or 213-214, having an X-ray powder diffraction pattern comprising a peak at about 3.8° 2θ.

Embodiment 216. The crystalline Compound 1 fumarate or hydrate or solvate thereof or the crystalline Compound 1 mono(fumarate) or hydrate or solvate thereof of any one of Embodiments 147, 206-207, or 213-214, having an X-ray powder diffraction pattern comprising peaks at about 3.8° 2θ and about 12.7° 2θ.

Embodiment 217. The crystalline Compound 1 fumarate or hydrate or solvate thereof or the crystalline Compound 1 mono(fumarate) or hydrate or solvate thereof of any one of Embodiments 147, 206-207, or 213-217, having an X-ray powder diffraction pattern substantially the same as that of FIG. 135.

Embodiment 218. Crystalline Form 3 Compound 1 fumarate or hydrate or solvate thereof.

Embodiment 219. The crystalline Form 3 Compound 1 fumarate or hydrate or solvate thereof of Embodiment 218, wherein the crystalline Form 3 Compound 1 fumarate or hydrate or solvate thereof is crystalline Form 3 Compound 1 mono(fumarate) or hydrate or solvate thereof.

Embodiment 220. The crystalline Compound 1 fumarate or hydrate or solvate thereof or the crystalline Compound 1 mono(fumarate) or hydrate or solvate thereof of any one of Embodiments 147, 206-207, or 218-219, having an X-ray powder diffraction pattern comprising a peak at about 8.8° 2θ.

Embodiment 221. The crystalline Compound 1 fumarate or hydrate or solvate thereof or the crystalline Compound 1 mono(fumarate) or hydrate or solvate thereof of any one of Embodiments 147, 206-207, or 218-219, having an X-ray powder diffraction pattern comprising peaks at about 8.8° 2θ and about 15.8° 2θ.

Embodiment 222. The crystalline Compound 1 fumarate or hydrate or solvate thereof or the crystalline Compound 1 mono(fumarate) or hydrate or solvate thereof of any one of Embodiments 147, 206-207, or 218-221, having an X-ray powder diffraction pattern substantially the same as that of FIG. 137.

Embodiment 223. Crystalline Compound 1 L-malate or hydrate or solvate thereof.

Embodiment 224. Crystalline Compound 1 mono(L-malate) or hydrate or solvate thereof.

Embodiment 225. The crystalline Compound 1 L-malate or hydrate or solvate thereof or the crystalline Compound 1 mono(L-malate) or hydrate or solvate thereof of any one of Embodiments 146, 223, or 224, having an X-ray powder diffraction pattern comprising a peak at about 4.3° 2θ.

Embodiment 226. The crystalline Compound 1 L-malate or hydrate or solvate thereof or the crystalline Compound 1 mono(L-malate) or hydrate or solvate thereof of any one of Embodiments 146, 223, or 224, having an X-ray powder diffraction pattern comprising one or more peaks at about 4.3° 2θ, about 10.1° 2θ, about 11.4° 2θ, and about 12.8° 2θ.

Embodiment 227. The crystalline Compound 1 L-malate or hydrate or solvate thereof or the crystalline Compound 1 mono(L-malate) or hydrate or solvate thereof of any one of Embodiments 146 or 223-226, having an X-ray powder diffraction pattern substantially the same as that of FIG. 144.

Embodiment 228. Crystalline Compound 1 benzoate or hydrate or solvate thereof.

Embodiment 229. Crystalline Compound 1 mono(benzoate) or hydrate or solvate thereof.

Embodiment 230. Crystalline Form 1 Compound 1 benzoate or hydrate or solvate thereof.

Embodiment 231. The crystalline Form 1 Compound 1 benzoate or hydrate or solvate thereof of Embodiment 230, wherein the crystalline Form 1 Compound 1 benzoate or hydrate or solvate thereof is crystalline Form 1 Compound 1 mono(benzoate) or hydrate or solvate thereof.

Embodiment 232. The crystalline Compound 1 benzoate or hydrate or solvate thereof or the crystalline Compound 1 mono(benzoate) or hydrate or solvate thereof of any one of Embodiments 143 or 228-231, having an X-ray powder diffraction pattern comprising a peak at about 4.9° 2θ.

Embodiment 233. The crystalline Compound 1 benzoate or hydrate or solvate thereof or the crystalline Compound 1 mono(benzoate) or hydrate or solvate thereof of any one of Embodiments 143 or 228-231, having an X-ray powder diffraction pattern comprising peaks at about 4.9° 2θ and about 11.7° 2θ.

Embodiment 234. The crystalline Compound 1 benzoate or hydrate or solvate thereof or the crystalline Compound 1 mono(benzoate) or hydrate or solvate thereof of any one of Embodiments 143 or 228-233, having an X-ray powder diffraction pattern substantially the same as that of FIG. 148.

Embodiment 235. Crystalline Form 2 Compound 1 benzoate or hydrate or solvate thereof.

Embodiment 236. The crystalline Form 2 Compound 1 benzoate or hydrate or solvate thereof of Embodiment 235, wherein the crystalline Form 2 Compound 1 benzoate or hydrate or solvate thereof is crystalline Form 2 Compound 1 mono(benzoate) or hydrate or solvate thereof.

Embodiment 237. The crystalline Compound 1 benzoate or hydrate or solvate thereof or the crystalline Compound 1 mono(benzoate) or hydrate or solvate thereof of any one of Embodiments 143, 228-229, or 235-236, having an X-ray powder diffraction pattern comprising a peak at about 3.8° 2θ.

Embodiment 238. The crystalline Compound 1 benzoate or hydrate or solvate thereof or the crystalline Compound 1 mono(benzoate) or hydrate or solvate thereof of any one of Embodiments 143, 228-229, or 235-237, having an X-ray powder diffraction pattern substantially the same as that of FIG. 150.

Embodiment 239. Crystalline Form 3 Compound 1 benzoate or hydrate or solvate thereof.

Embodiment 240. The crystalline Form 3 Compound 1 benzoate or hydrate or solvate thereof of Embodiment 239, wherein the crystalline Form 3 Compound 1 benzoate or hydrate or solvate thereof is crystalline Form 3 Compound 1 mono(benzoate) or hydrate or solvate thereof.

Embodiment 241. The crystalline Compound 1 benzoate or hydrate or solvate thereof or the crystalline Compound 1 mono(benzoate) or hydrate or solvate thereof of any one of Embodiments 143, 228-229, or 239-240, having an X-ray powder diffraction pattern comprising a peak at about 7.2° 2θ.

Embodiment 242. The crystalline Compound 1 benzoate or hydrate or solvate thereof or the crystalline Compound 1 mono(benzoate) or hydrate or solvate thereof of any one of Embodiments 143, 228-229, or 239-241, having an X-ray powder diffraction pattern substantially the same as that of FIG. 152.

Embodiment 243. Crystalline Form 7 Compound 1 hydrogen phosphate or hydrate or solvate thereof.

Embodiment 244. The crystalline Form 7 Compound 1 hydrogen phosphate or hydrate or solvate thereof of Embodiment 243, wherein the crystalline Form 7 Compound 1 hydrogen phosphate or hydrate or solvate thereof is crystalline Form 7 Compound 1 di(hydrogen phosphate) or hydrate or solvate thereof.

Embodiment 245. The crystalline Compound 1 hydrogen phosphate or hydrate or solvate thereof or the crystalline Compound 1 di(hydrogen phosphate) or hydrate or solvate thereof of any one of Embodiments 140C, 149, 165, 180, or 243-244, having an X-ray powder diffraction pattern comprising a peak at about 3.6° 2θ.

Embodiment 246. The crystalline Compound 1 hydrogen phosphate or hydrate or solvate thereof or the crystalline Compound 1 di(hydrogen phosphate) or hydrate or solvate thereof of any one of Embodiments 140C, 149, 165, 180, or 243-244, having an X-ray powder diffraction pattern comprising one or more peaks at about 3.6° 2θ, about 12.1° 2θ, and about 20.1° 2θ.

Embodiment 247. The crystalline Compound 1 hydrogen phosphate or hydrate or solvate thereof or the crystalline Compound 1 di(hydrogen phosphate) or hydrate or solvate thereof of any one of Embodiments 140C, 149, 165, 180, or 243-246, having an X-ray powder diffraction pattern substantially the same as that of FIG. 158.

Embodiment 248. A substantially crystalline salt of Compound 1 or a hydrate or solvate thereof.

Embodiment 249. The substantially crystalline salt or hydrate or solvate thereof of Compound 1 of Embodiment 248, wherein the substantially crystalline salt or hydrate or solvate thereof is more than 50% crystalline.

Embodiment 250. A salt or hydrate or solvate thereof selected from Compound 1 hydrogen chloride, Compound 1 hydrogen phosphate, Compound 1 tosylate, Compound 1 mesylate, Compound 1 benzenesulfonate, Compound 1 L-tartrate, Compound 1 fumarate, Compound 1 citrate, Compound 1 maleate, Compound 1 L-malate, Compound 1 benzoate, and hydrates and solvates thereof.

Embodiment 251. A composition comprising two or more salts of Compound 1 or a hydrate or solvate thereof of any one of Embodiments 134-135 or 137-250.

Embodiment 252. The composition of Embodiment 251, wherein at least one of the two or more salts of Compound 1 or hydrates or solvates thereof is crystalline.

Embodiment 253. A pharmaceutical composition comprising a salt of Compound 1 or a hydrate or solvate thereof of any one of Embodiments 134-135 or 137-250 and at least one pharmaceutically acceptable excipient.

Embodiment 254. The pharmaceutical composition of Embodiment 253, wherein the salt of Compound 1 or hydrate or solvate thereof is crystalline.

Embodiment 255. A dosage form comprising a pharmaceutical composition of Embodiment 253 or 254.

Embodiment 256. The dosage form of Embodiment 255, wherein the dosage form is a solid dosage form.

Embodiment 257. The dosage form of Embodiment 255 or 256, wherein the dosage form is an oral dosage form.

Embodiment 258. A method of inhibiting ENT1 in a patient need thereof, comprising: administering to said patient an effective amount of a salt of Compound 1 or hydrate or solvate thereof of any one of Embodiments 134-135 or 137-250; an effective amount of a composition or pharmaceutical composition according to any one of Embodiments 251-254; or a dosage form according to any one of Embodiments 255-257.

Embodiment 259. Use of a salt of Compound 1 or hydrate or solvate thereof of any one of Embodiments 134-135 or 137-250; a composition or pharmaceutical composition according to any one of Embodiments 251-254; or a dosage form according to any one of Embodiments 255-257, for the manufacture of a medicament for inhibiting ENT1.

Embodiment 260. A salt of Compound 1 or hydrate or solvate thereof of any one of Embodiments 134-135 or 137-250; a composition or pharmaceutical composition according to any one of Embodiments 251-254; or a dosage form according to any one of Embodiments 255-257, for use as a medicament.

Embodiment 261. A method of treating cancer in a patient need thereof, comprising: administering to said patient an effective amount of a salt of Compound 1 or hydrate or solvate thereof of any one of Embodiments 134-135 or 137-250; an effective amount of a composition or pharmaceutical composition according to any one of Embodiments 251-254; or a dosage form according to any one of Embodiments 255-257.

Embodiment 262. Use of a salt of Compound 1 or hydrate or solvate thereof of any one of Embodiments 134-135 or 137-250; a composition or pharmaceutical composition according to any one of Embodiments 251-254; or a dosage form according to any one of Embodiments 255-257, for the manufacture of a medicament for treating cancer.

Embodiment 263. A salt of Compound 1 or hydrate or solvate thereof of any one of Embodiments 134-135 or 137-250; a composition or pharmaceutical composition according to any one of Embodiments 251-254; or a dosage form according to any one of Embodiments 255-257, for use in a method of treating cancer.

Embodiment 264. A method of treating cancer in a patient need thereof, comprising: administering to said patient (1) an effective amount of a salt of Compound 1 or hydrate or solvate thereof of any one of Embodiments 134-135 or 137-250; an effective amount of a composition or pharmaceutical composition according to any one of Embodiments 251-254; or a dosage form according to any one of Embodiments 255-257; and (2) an adenosine receptor antagonist.

Embodiment 265. Use of a combination of (1) a salt of Compound 1 or hydrate or solvate thereof of any one of Embodiments 134-135 or 137-250; a composition or pharmaceutical composition according to any one of Embodiments 251-254; or a dosage form according to any one of Embodiments 255-257; and (2) an adenosine receptor antagonist for the manufacture of a medicament for treating cancer.

Embodiment 266. The method or use of Embodiment 264 or 265, wherein (1) the salt of Compound 1 or hydrate or solvate thereof of any one of Embodiments 134-135 or 137-250; the composition or pharmaceutical composition according to any one of Embodiments 251-254; or the dosage form according to any one of Embodiments 255-257, is administered prior to, concomitant with, or subsequent to administration of (2) the adenosine receptor antagonist.

Embodiment 267. The method or use of any one of Embodiments 264-266, wherein the adenosine receptor antagonist is an A2A or A2B receptor antagonist.

Embodiment 268A. The method or use of any one of Embodiments 264-267, wherein the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine;
4-hydroxy-N-(4-methoxy-7-morpholinobenzo[d]thiazol-2-yl)-4-methylpiperidine-1-carboxamide;
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
   and pharmaceutical salts and solvates thereof.

Embodiment 268B. The method or use of any one of Embodiments 264-267, wherein the adenosine receptor antagonist is a hydrochloride salt of (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one.

Embodiment 268C. The method or use of Embodiment 268B, wherein the adenosine receptor antagonist is the hydrochloride salt of (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one is Form 2.

### Examples

### Abbreviations and Definitions

| | |
|---|---|
| 5EU | 5-Ethynyl-Uridine |
| ACE | Acetone |
| ACN | Acetonitrile |
| ASA | Anti-solvent addition |
| a_{w} | Water activity |
| ca. | Circa/approximately |
| DCM | Dichloromethane |
| DCC | dicyclohexylcarbodiimide |
| DEAD | Diethyl azodicarboxylate |
| DIBAL-H | Diisobutylaluminium hydride |
| DIC | N,N′-Diisopropylcarbodiimide |
| DIEA | N,N-Diisopropylethylamine |
| DMAP | 4-Dimethylaminopyridine |
| DMF | Dimethylformamide |
| DMSO | Dimethylsulfoxide |
| DSC | Differential Scanning Calorimetric Analysis |
| DVS | Dynamic Vapor Sorption |
| EtOAc | Ethyl acetate |
| EtOH | Ethanol |
| equiv. | Equivalence |
| FaSSGF | Fasted State Simulated Gastric Fluid |
| FaSSIF | Fasted State Simulated Intestinal Fluid |
| FeSSIF | Fed State Simulated Intestinal Fluid |
| FT-IR | Fourier-Transform Infrared Spectroscopy |
| h or hr | Hour |
| HCl | Hydrogen chloride acid (also known as |
| | hydrochloric acid) |
| HPLC | High Performance Liquid Chromatography |
| HPLC-CAD | High Performance Liquid Chromatography - |
| | Charged Aerosol Detection |
| HPLC-UV | High Performance Liquid Chromatography - |
| | Ultraviolet Detection |
| IPA | Isopropyl alcohol |
| IR | Infrared Spectroscopy |
| KF | Karl Fischer Coulometric Titration |
| L | Liters |
| LCMS | Liquid chromatography-mass spectrometry |
| Max. | Maximum |
| MEK | Methyl ethyl ketone (also known as butanone) |
| MeOH | Methanol |
| 2-Methyl THF | 2-Methyltetrahydrofuran |
| or 2-MeTHF | |
| MiBK | Methyl Isobutyl Ketone |
| Min. | Minutes |
| mg | Milligrams |
| mL | Milliliters |
| MSZW | Metastable Zone Width |
| MTBE | Methyl tertiary-butyl ether |
| N/A | Not applicable |
| NMR | Nuclear Magnetic Resonance Spectroscopy |
| PLM | Polarized light microscopy |
| ppm | Parts per million |
| PyBop | Benzotriazol-1-yloxytripyrrolidinophosphonium |
| | hexafluorophosphate |
| RT | Room temperature |
| RH | Relative humidity |
| RPM | Revolutions per minute |
| SFC | Supercritical Fluid Chromatography |
| (S,S)-Ms-DENEB | Chloro[(S,S)-N-[2-(4-methylbenzyloxy)ethyl]- |
| catalyst | N′-(p-toulenesulfonyl)-1,2-diphenyleth- |
| | ylenediamine]ruthenium (II) |
| tBME | tert-Butyl methyl ether |
| TEA | Triethylamine |
| TG/DSC | Thermogravimetric/Differential Scanning Calorimetry |
| TGA/DSC | Thermogravimetric Analysis/Differential Scanning |
| | Calorimetry |
| TGA-IR | Thermogravimetric Analysis Infrared Spectroscopy |
| THF | tetrahydrofuran |
| μL | Microliters |
| Vol. | Volume |
| VH-XRPD | Variable Humidity X-ray Powder Diffraction |
| VT-XRPD | Variable Temperature X-ray Powder Diffraction |
| v/v | Volume/volume |
| w/w | Weight/weight |
| XRPD | X-ray Powder Diffraction |

### Examples—Sulfuric Acid

### Analytical Methods

### (1) X-Ray Powder Diffraction (XRPD)

In all examples below except Example 10, unless otherwise stated, XRPD analysis was carried out on a PANalytical X′pert pro with PIXcel detector (128 channels), scanning the samples between 3 and 35° 2θ. The material was gently ground (where required) to release any agglomerates and loaded onto a multi-well plate with Mylar polymer film to support the sample. The multi-well plate was then placed into the diffractometer and analyzed using Cu K radiation (α1 λ=1.54060 Å; α2=1.54443 Å; β-1.39225 Å; α1: α2 ratio=0.5; most, if not all, β radiation is removed from the beam using an X-ray mirror) running in transmission mode (step size 0.0130° 2θ, step time 18.87s) using 40 kV/40 mA generator settings. Data were visualized and images generated using the HighScore Plus 4.7 desktop application (PANalytical, 2017).

In Example 10, NMR data was obtained on a Bruker DPX 400 Mz spectrometer.

### (2) Single Crystal X-Ray Diffraction (SCXRD)

In Example 6, a suitable crystal was selected and mounted in a nylon loop while protected in a protective layer of paratone oil. Two data sets were collected at temperatures: 295K and 100K. In both cases, data were collected using a Bruker D8 Venture diffractometer equipped with a Photon III detector operating in shutterless mode with Cu-Kα radiation (1.54178 Å). All nonhydrogen atoms were located in the Fourier map and their positions refined prior to describing the thermal movement of all non-hydrogen atoms anisotropically.

The structures were solved in the Olex2 software package with the ShelXT (intrinsic phasing) structure solution program and refined with the ShelXL refinement package using Least Squares minimization. See Dolomanov, O. V. et al, J. Appl. Cryst., 2009, 42, 339-341; Sheldrick, G. M., Acta Cryst., 2015, A71, 3-8; and Sheldrick, G. M., Acta Cryst., 2015, C71, 3-8. Data was collected, solved, and refined in triclinic space-group P1.

A series of restraints were applied to the final refinement. All CH and CH₂ groups were refined with fixed Uiso of 1.2 times, and all CH₃ groups with fixed Uiso of 1.5 times. The anions were disordered in the structure collected at 100K and they were modelled as follows: —S4 was disordered over two positions and modelled at 0.44 occupancy; O300, O302 and O303 were disordered over two positions and modelled at 0.55 occupancy.

For the structure collected at 295 K, the highest residual Fourier peak was found to be 0.75 e. Å⁻³ approx. 2.43 Å from O302 and the deepest Fourier hole was found to be −0.72 e. Å⁻³ approx. 0.62 Å from S2.

For the structure collected at 100 K, the highest residual Fourier peak was found to be 0.63 e. Å⁻³ approx. 0.51 Å from H4WA and the deepest Fourier hole was found to be −1.17 e.Å⁻³ approx. 0.61 Å from S2.

Crystal data for the structure at 295K was obtained with the following parameters: C₃₃H₅₅N₃O₁₉S₂* (M=858.89 g/mol): triclinic, space group P1 (no. 1), a=9.669(2) Å, b=12.439(3) Å, c=18.717(4) Å, α=102.143(13)°, β=94.505(14)°, γ=110.746(12)°, V=2029.0(8) Å³, Z=2 T=295(2) K, μ(CuKα)=1.896 mm⁻¹, Dcalc=1.406 g/cm³, 59429 reflections measured (4.898≤2Θ≤133.718), 13898 unique (Rᵢₙₜ=0.0790, R_{sigma}=0.0782) which were used in all calculations. The final R₁ was 0.0835 (>2σ(I)) and wR2 was 0.2767 (all data). * the calculated formula differs from the reported formula by two hydrogen atoms and one oxygen atom that were not located

Crystal data for the structure at 100K was obtained with the following parameters: C₃₃H₅₇N₃O₂₀S₂ (M=879.93 g/mol): triclinic, space group P₁ (no. 1), a=9.6794(2) Å, b=12.3886(2) Å, c=18.4016(4) Å, α=101.6708(8)°, β=94.7448(8)°, γ=110.7514(8)°, V=1991.88(7) Å³, Z=2, T=100.0 K, μ(CuKα)=1.962 mm-1, Dcalc=1.467 g/cm3, 97841 reflections measured (4.974°≤2Θ≤144.29°), 15198 unique (Rᵢₙₜ=0.0299, R_{sigma}=0.0202) which were used in all calculations. The final R₁ was 0.0586 (I>2σ(I)) and wR₂ was 0.1597 (all data).

### (3) Variable Temperature X-Ray Powder Diffraction (VT-XRPD)

In the examples below, VT-XRPD analysis was carried out on a Philips X'Pert Pro Multipurpose diffractometer equipped with a temperature chamber. The samples were scanned between 4 and 36° 2θ using Cu K radiation (α1 λ=1.54060 Å; α2=1.54443 Å; β=1.39225 Å; α1: α2 ratio=0.5; most, if not all, B radiation is removed from the beam using an X-ray mirror) running in Bragg-Brentano geometry (step size 0.008° 2θ) using 40 kV/40 mA generator settings. Measurements were carried out from ambient temperature to an upper limit of 250° C., with a final measurement taken after cooling.

### (4) Variable Humidity X-Ray Powder Diffraction (VH-XRPD)

In the examples below, VH-XRPD analysis was carried out on a Philips X'Pert Pro Multipurpose diffractometer equipped with a humidity chamber. The samples were scanned between 4 and 36° 2θ using Cu K radiation (α1λ=1.54060 Å; α2=1.54443 Å; β=1.39225 Å; α1: α2 ratio=0.5; most, if not all, B radiation is removed from the beam using an X-ray mirror) running in Bragg-Brentano geometry (step size 0.008° 2θ) using 40 kV/40 mA generator settings. Measurements were carried out from 2-90% RH, with a final measurement taken at 40% RH.

(5) Thermogravimetric Analysis/Differential Scanning calorimetry (TGA/DSC), a.k.a. Thermogravimetric/Differential Scanning calorimetry (TG/DSC)

In the examples below, to measure TGA/DSC, 2-10 mg of material was added into a pre-tared open aluminum pan and loaded into a TA Instruments Discovery SDT 650 Auto—Simultaneous DSC and held at room temperature. The sample was then heated at a rate of 10° C./min from 30° C. to 400° C. during which time the change in sample weight was recorded along with the heat flow response (DSC). Nitrogen was used as the sample purge gas, at a flow rate of 200 cm³/min.

(6) Differential Scanning calorimetry (DSC)

METHOD A: In Example 3, 1-5 mg of material was weighed into an aluminum DSC pan and sealed non-hermetically with a pierced aluminum lid. The sample pan was then loaded into a TA DSC2500 and held at 20° C. Once a stable heat-flow response was obtained, the sample and reference were heated to an upper temperature of 200° C. at a scan rate of 10° C./min and the resulting heat flow response monitored. The sample was held at the upper temperature for 3 minutes, before it was cooled at 10° C./min to 20° C., reheated to the upper temperature at 10° C./min, and then cooled again to 20° C. at the same rate. Nitrogen was used as the purge gas, at a flow rate of 50 cm³/min.

METHOD B: In Examples 2A, 14, and 17, 1-5 mg of material was weighed into an aluminum DSC pan and sealed non-hermetically with a pierced aluminum lid. The sample pan was then loaded into a TA DSC2500 and held at 20° C. Once a stable heat-flow response was obtained, the sample and reference were heated to an upper temperature of 200° C. at a scan rate of 10° C./min and the resulting heat flow response monitored. The sample was held at the upper temperature for 3 minutes, before it was cooled at 10° C./min to −80° C. and then reheated to the upper temperature at 10° C./min. Nitrogen was used as the purge gas, at a flow rate of 50 cm³/min.

METHOD C: In Example 5, 1-5 mg of a sample were weighed into an aluminum DSC pan and sealed non-hermetically with an aluminum lid. The sample pan was then loaded into a TA Instruments Discovery DSC 2500 differential scanning calorimeter equipped with a RC90 cooler. The sample and reference were heated to 190° C. at a scan rate of 10° C./min and the resulting heat flow response monitored. The sample was re-cooled to −80° C. and then reheated again to 190° C. all at 10° C./min. Nitrogen was used as the purge gas, at a flow rate of 50 cm³/min.

### (7) Nuclear Magnetic Resonance Spectroscopy (NMR)

METHOD A: In Examples 2A, 2B, 3, 7, 14, and 17, 1H NMR experiments were performed on a Bruker AV500 (frequency: 500 MHz for protons). Experiments were performed in d₄-methanol and samples were prepared to ca. 5 mM concentration.

METHOD B: In Example 5, NMR experiments were performed on a Bruker A VIIIHD spectrometer equipped with a DCH cryoprobe operating at 500.12 MHz for protons. Experiments were performed in d4-methanol and each sample was prepared to ca. 10 mM concentration. Quantitative NMR analysis was carried out using 1,3,5-trimethoxybenzene as the external standard; experiments were carried out in d₄-methanol for Compound 1, which is a free base, samples and in DMSO-d₆ for the di(hydrogen sulfate) samples.

METHOD C: In Example 10, NMR data was obtained on a Bruker DPX 400 Mz spectrometer.

### (8) Infrared Spectroscopy (IR)

In the examples below, Infrared spectroscopy was carried out on a Bruker

ALPHA P spectrometer. Sufficient material was placed onto the center of the plate of the spectrometer and the spectra were obtained using the following parameters:
Resolution: 4 cm⁻¹
Background Scan Time: 16 scans
Sample Scan Time: 16 scans
Data Collection: 4000 to 400 cm⁻¹
Result Spectrum: Transmittance Software: OPUS version 6

### (9) Polarized Light Microscopy (PLM)

In Example 5, the presence of crystallinity (birefringence) was determined using an Olympus BX50 microscope, equipped with cross-polarizing lenses and a Motic camera. Images were captured using Motic Images Plus 2.0. All images were recorded using the 20× objective, unless otherwise stated.

### (10) Hot Stage Light Microscopy (HSM)

In Example 11, thermal events were monitored visually using a calibrated Linkam THM600 hotstage with connected controller unit coupled to an Olympus BH2 microscope equipped with a Motic camera and image capture software (Motic Images Plus 3.0). Approximately 0.5 mg of material was placed onto a microscope coverslip and heated at a rate of 10° C./min with images taken at routine intervals to document any thermal transitions. All images were recorded using the 20× objective, unless otherwise stated.

### (11) Karl Fischer Coulometric Titration (KF)

In the examples below, KF titration measured using either an In Motion KF Autosampler or Direct Addition Method. The In Motion KF Autosampler was carried out via the following procedure. 15-20 mg of solid was weighed into a 10 mL glass vial and tightly sealed with a screw cap. Samples were analyzed using a Mettler Toledo C30SX and an InMotion KFOven Autosampler at 130° C. Samples were analyzed in duplicate and an average moisture content reported. See table below for further details.

| | | |
|---|---|---|
| Blank | Oven Temperature/° C. | 130 |
| | Source for Drift | Determination |
| | Max. Start Drift/μg/min | 10 |
| | Carrier Gas Flow Rate/mL/min | 80 |
| | Transfer Tube Heating | No |
| | Mix Time/s | 60 |
| | Stir Speed/% | 45 |
| | Drift Termination/s | 10 (Delay Time) |
| | Max. Titration Time/s | 600 |
| Sample | Oven Temperature/° C. | 130 |
| | Source for Drift | Determination |
| | Max. Start Drift/μg/min | 10 |
| | Carrier Gas Flow Rate/mL/min | 80 |
| | Mix Time/s | 60 |
| | Stir Speed/% | 45 |
| | Drift Termination/s | 10 (Delay Time) |
| | Max. Titration Time/s | 600 |

The Direct Addition Method was carried out via the following procedure. 15-20 mg of solid material was accurately weighed into a vial. The solid was then manually introduced into the titration cell of a Mettler Toledo C30 Compact Titrator. The vial was back-weighed after the addition of the solid and the weight of the added solid entered on the instrument. Titration was initiated once the sample had fully dissolved in the cell. The water content was calculated automatically by the instrument as a percentage and the data printed.

### (12) Dynamic Vapor Sorption (DVS)

METHOD A: In Example 11, approximately 5-15 mg of sample was placed into a mesh vapor sorption balance pan and loaded into either a DVS Intrinsic or DVS Advantage dynamic vapor sorption balance by Surface Measurement Systems. The sample was subjected to a ramping profile from 40-90% relative humidity (RH) at 10% increments, maintaining the sample at each step until a stable weight had been achieved (dm/dt 0.004%, minimum step length 30 minutes, maximum step length 500 minutes) at 25° C. After completion of the sorption cycle, the sample was dried using the same procedure to 0% RH and then a second sorption cycle back to 40% RH was carried out. Two cycles were performed. The weight change during the sorption/desorption cycles were plotted, allowing the hygroscopic nature of the sample to be determined. XRPD analysis was then carried out on the residual solid.

METHOD B: In Examples 2A, 3, 14, and 17, approximately 10-20 mg of sample was placed into a mesh vapor sorption balance pan and loaded into either a DVS Intrinsic or DVS Advantage dynamic vapor sorption balance by Surface Measurement Systems. The sample was subjected to a ramping profile from 40-90% relative humidity (RH) at 10% increments, maintaining the sample at each step until a stable weight had been achieved (dm/dt 0.004%, minimum step length 30 minutes, maximum step length 500 minutes) at 25° C. After completion of the sorption cycle, the sample was dried using the same procedure to 0% RH and then a second sorption cycle back to 40% RH was carried out. Two cycles were performed. The weight change during the sorption/desorption cycles were plotted, allowing the hygroscopic nature of the sample to be determined.

### (13) Appearance Testing

In Example 12, the solid sample, in a clear, colorless, glass vial, was examined against a matt white background. The color of the material was determined with reference to the Sigma Aldrich colour chart.

### (14) Liquid Chromatography-Mass Spectrometry (LCMS)

In Example 10, LCMS was performed using one of the following methods:

### Method A:

Instrument: Agilent 1200 HPLC MSD:6120 single quadrupole MSD
Column: Luna C18, 2.0*50 mm, 5 μm
Column temperature: 40° C.
Mobile Phase A (MP A): 0.04% TFA in H₂O
Mobile Phase B (MP B): 0.02% TFA in ACN
Flow Rate: 1.0 mL/min
Detection: 220 nm
Gradient Ratio:

| Time (min) | MP A (%) | MP B (%) |
|---|---|---|
| 0.01 | 95 | 5 |
| 0.40 | 95 | 5 |
| 3.00 | 5 | 95 |
| 4.00 | 5 | 95 |
| 4.01 | 95 | 5 |
| 4.50 | 95 | 5 |

### Method B:

Instrument: Shimadzu LC-20AD MSD:LCMS-2020
Column: Kinetex 5 um EVO C18 30*2.1 mm
Column temperature: 40° C.
Mobile Phase A (MP A): 0.04% TFA in H₂O
Mobile Phase B (MP B): 0.02% TFA in ACN
Flow Rate: 1.5 mL/min

| Time (min) | MP B (%) | Flow (ml/min) |
|---|---|---|
| 0.01 | 5 | 1.5 |
| 0.70 | 95 | 1.5 |
| 1.16 | 95 | 1.5 |
| 1.50 | 5 | 1.5 |

### (15) High Performance Liquid Chromatography-Ultraviolet Detection (HPLC-UV)

Unless otherwise indicated in the examples below, HPLC-UV was performed with the follow experimental parameters and conditions:
Column: Waters XSelect CSH Fluoro-Phenyl 150×4.6 mm, 2.5 μm
Mobile Phase A: 0.1% TFA in water
Mobile Phase B: 0.1% TFA in acetonitrile
Diluent: Methanol
Autosampler Temperature: 5° C.
Flow Rate: 1.0 mL/min
Runtime: 30 minutes
Column Temperature: 30° C. (±1° C.)
Column Pressure: 110 bar (at start of run)
Injection Volume: 5 μL
Sample Concentration: 0.5 mg/mL
Detection: 266 nm
Sampling Rate: 50 Hz
Gradient Program:

| Time (min) | Mobile Phase A (%) | Mobile Phase B (%) |
|---|---|---|
| 0.0 | 95 | 5 |
| 5.0 | 75 | 25 |
| 18.0 | 65 | 35 |
| 20.0 | 50 | 50 |
| 22.0 | 5 | 95 |
| 25.0 | 5 | 95 |
| 25.1 | 95 | 5 |
| 30.0 | 95 | 5 |

### (16) Chiral High-Performance Liquid Chromatography (Chiral HPLC)

Unless otherwise indicated in the examples below, chiral HPLC was performed using one of the two methods below:

### Method 1:

Instrument: CAS-TJ-Chiral HPLC-K (Waters Arc with PDA detector)
Proc. Chnl. Descr.: 2998 PDA 254.0 nm (2998 (190-300)nm)
Column: Chiralpak IC-3, 50×4.6 mm, I.D., 3 um
Mobile phase: A: Heptane B: EtOH (0.05% DEA, v/v)
Gradient: A:B=20:80
Flow rate: 1 mL/min
Column temp.: 35° C.

### Method 2:

Instrument: CAS-TJ-ANA-Chiral HPLC-K (Waters Arc with 2998)
Proc. Chnl. Descr.: 2998 PDA 254.0 nm (2998 (190-300)nm)
Column: Chiralpak IF-3, 150×4.6 mm I.D., 3 um
Mobile phase: A: Heptane B: EtOH+ACN (4:1) (0.05% IPAm, v/v)
Gradient: A:B=92:8
Flow rate: 1 mL/min
Column temp.: 30° C.

### (17) Gas Chromatography (GC)

In Example 5, gas chromatography was conducted using a Thermo Trace 1300 GC equipped with a Tri-Plus 300 Headspace sampler. The following parameters were used:
Column: Agilent J&W DB-624 30 m×0.32 mm, 1.8 um d.f. or equivalent
Oven Temperature: 35° C. (hold 0.5 min) to 45° C. @ 16.5° C./min to 70° C. @ 5.0° C./min to 220° C.@30.0° C./min
Flow Rate: 2.2 mL/min (constant flow)
Carrier Gas: Hydrogen
Diluent: DMA
Injection Mode: Split
Injection Temperature: 225° C.
Injection Split Ratio: 20:1
Detector Temperature: 250° C.
Detector Hydrogen: 30.0 mL/min
Detector Air: 400 mL/min
Make-up Flow: 40.0 mL/min
Make-up Gas: Nitrogen
The following Headspace Parameters were used:
Oven Temperature: 100° C.
Loop Temperature: 110° C.
Transfer Line Temperature: 150° C.
Vial Equilibration Time: 10.0 min
Pressurization Mode: Pressure
Auxiliary Pressure: 100 kPa (Nitrogen)
Pressurization Time: 0.2 min
Loop Fill Mode: Pressure
Loop Pressure: 50 kPa
Loop Equilibration Time: 0.2 min
Loop Volume: 1 mL
Inject Time: 0.5 min
Vial Shaking: High
GC Cycle Time: 21 min.

### (18) Raman Spectroscopy

In Example 4, Raman spectroscopy was carried out using a Wasatch 785 nm spectrometer and a BlazeMetrics 900 probe. The material to be analyzed was brought in contact with the probe window and measurements were taken with the following parameters:
Wavenumber: ca. 2021-218 cm⁻¹
Accumulations: 2
Integration time: 2000 ms

Care was taken to protect the sample from external light sources.

### (19) Particle Size Distribution

In Example 5, the particle size distribution of solids was measured using a Mastersizer 2000 instrument and the method is detailed below.

| Dispersant | 0.05% w/v span 85 in Heptane | |
|---|---|---|
| Concentration | 100 mg in 10 mL dispersant | |
| Stirrer Speed | 2000 | rpm |
| Sonication time | 30 | seconds |
| Pre-measurement delay | 1 | minute |
| Measurement time | 10 | seconds |
| Background measurement time | 10 | seconds |

| | |
|---|---|
| Measurement cycles | 3 |
| Obscuration | 5-20% |
| Analysis model | General model |
| Sensitivity | Normal |
| Dispersant Refractive Index | 1.39 |
| Material size absorption | 0.01 |
| Material R.I. | 1.58 |

### (20) High Throughput-Xray Powder Diffraction (HT-XRPD)

The HT-XRPD patterns referenced in Example 19 were as follows. The plates were mounted on a Bruker General Area Detector Diffraction System (GADDS) equipped with a VÅNTEC-500 gas area detector corrected for intensity and geometric variations. The calibration of the measurement accuracy (peaks position) was performed using NIST SRM1976 standard (Corundum).

Data collection was carried out at room temperature using monochromatic Cu Kα radiation in the 20 region (1.54178 Å) between 1.5° and 41.5°. The diffraction pattern of each well was collected in two 20 ranges (1.5°≤2θ≤21.5° for the first frame, and 19.5°≤2θ≤41.5° for the second) with an exposure time of 90s for each frame. No background subtraction or curve smoothing was applied to the XRPD patterns.

### (21) Ultra-Performance Liquid Chromatography (UPLC)

UPLC System—UPLC: ThermoFisher Vanquish, Detector 1: UV detector set at 266 nm

UPLC Conditions—
Auto sampler temp.: RT
Column: Agilent Eclipse Plus C18 HD (50×2.1 mm; 1.8 μm)
Column temp: 40° C.
Gradient: Mobile phase A: 10 mM ammonium acetate in water
Mobile phase B: Acetonitrile
Flow: 0.6 mL/min
Gradient:

| Time [min] | Eluent A | Eluent B |
|---|---|---|
| 0 | 95% | 5% |
| 0.1 | 95% | 5% |
| 2.5 | 3% | 97% |
| 2.55 | 3% | 97% |
| 2.56 | 95% | 5% |
| 3.5 | 95% | 5% |

Run time: 4.0 minutes

### Sample Preparation—

Concentration: ca. 1.0 mg/mL
Solvent: ACN/water 50:50
Injection volume: 1 μl
Retention time: 2.9 min

The compound integrity is expressed as a peak-area percentage, calculated from the area of each peak in the chromatogram, except the ‘injection peak’, and the total peak-area, as follows:

$$peak area ( \% ) = \frac{peak area}{total area of all peaks} \cdot 100 \%$$

The peak area percentage of the compound of interest is employed as an indication of the purity of the component in the sample.

Example 1—Solid Form Screen of Compound 1 Di(Hydrogen Sulfate): Amorphous Compound 1 di(hydrogen sulfate) (made in accordance with Example 7) was prepared and then subject to various crystallization techniques—temperature cycling, evaporation, crash cooling, solvent drop grinding, anti-solvent addition—each of which is described in more detail below. In addition, a reactive crystallization experiment was also conducted on Compound 1, which is a free base.

Temperature Cycling: Approximately 20 mg of Amorphous Compound 1 di(hydrogen sulfate) was added to 24×1.5 mL screw cap vials. Into each vial, the appropriate solvent system was added in 50 μL aliquots in an attempt to form slurries at 40° C. When the addition was completed, the samples were temperature-cycled between 40° C. and 5° C., using 0.1° C./min ramp and 1 hr hold between steps. After about 24 hours of cycling, anti-solvent addition at 40° C. was carried out on samples that were clear solutions. Temperature cycling was continued for all samples for a total of 48 hours. After 48 hours of cycling, any solids present in the samples were isolated via centrifugation and analyzed damp by XRPD to assess the polymorphic form. Table 35 below shows the solvent, anti-solvent and observations for the temperature cycling.

TABLE 35
Details of anti-solvent addition for temperature cycling experiments

Anti-solvent addition +
further cycling

| Solvent system | Anti-solvent | Observations |
|---|---|---|
| IPA:water | IPA | Slurry |
| (50:50% v/v) | | |
| DMSO | Ethyl acetate | Clear |
| Ethanol:water | Ethanol | Slurry |
| (50:50% v/v) | | |
| Methanol | tBME | Gum |
| DMF | tBME | Clear |

Evaporation: Solutions of Amorphous Compound 1 di(hydrogen sulfate) were prepared in different solvent systems. For solvent systems with high solubility, solutions were prepared by dissolving ca. 20 mg of Amorphous Compound 1 di(hydrogen sulfate) in 200 μL of the solvent at 25° C. For solvent systems with low solubility, slurries were prepared at 50° C. and the mother liquors were needle-filtered into a new set of vials. The solutions were then left open to allow the solvents to evaporate at ambient. Solids observed post-evaporation were analyzed by XRPD.

Crash Cooling: Solutions of Amorphous Compound 1 di(hydrogen sulfate) were prepared in different solvent systems. For solvent systems with high solubility, this was prepared by dissolving ca. 20 mg of the amorphous material in 200 μL of the solvent at 25° C. For solvent systems with low solubility, slurries were prepared at 50° C. and the mother liquors were needle-filtered into a new set of vials. The solutions were placed in a refrigerator to crash-cool the samples to about 5° C. for 48 hours. Solids observed at this point were analyzed by XRPD. The rest of the samples where no solids were observed were placed in a freezer to allow the samples to crash-cool further to about −20° C. After about 48 hours in the freezer, samples with solids were analyzed by XRPD. For samples where solids were still not observed, anti-solvent addition was carried out at 5° C., and the samples returned to the freezer. After 4 days in the freezer post-ASA, any solids present were analyzed by XRPD. Table 36 below shows the solvent, anti-solvent and observations for the crash cooling.

TABLE 36
Details of anti-solvent addition for crash cooling experiments

| | Anti-solvent Addition + further | |
|---|---|---|
| | cooling (−20° C.) | |
| Solvent system | Anti-solvent | Observations |
| 1,4-Dioxane | tBME | Clear |
| 2-Methyl THF | tBME | Clear |
| IPA:water (50:50% v/v) | IPA | Gum/slurry |
| Acetone | tBME | Clear |
| Acetone:water (75:25% v/v) | Acetone | Slurry |
| Acetonitrile | tBME | Clear |
| Acetonitrile:water (75:25% v/v) | Acetonitrile | Slurry |
| Dichloromethane (DCM) | tBME | Clear |
| DMSO | Ethyl acetate | Clear |
| Ethanol:water (50:50% v/v) | Ethanol | Clear |
| Ethyl Acetate | tBME | Clear |
| Isopropyl Acetate | tBME | Clear |
| Methanol | tBME | Gum |
| MEK | tBME | Clear |
| MiBK | tBME | Clear |
| DMF | tBME | Gum |
| THF | tBME | Few particles |
| Toluene | tBME | Cloudy |
| Water | Acetone | Clear |

Solvent Drop Grinding: 15-20 mg of Amorphous Compound 1 di(hydrogen sulfate) was added to 24×2 mL bead mill tubes. Into each tube, 5 μL of the appropriate solvent system was added at ambient. Two small stainless-steel beads were placed in each tube, and the samples were milled using the following program: (1) Speed=5000 rpm; (2) Cycle=10×90 s; (3) Pause=10 s; (4) Repeat=duplicate. For samples where dissolution was suspected during the milling, additional amorphous material was added, and the milling program repeated. When the milling program was completed, the residual solids were analyzed by XRPD.

Anti-solvent Addition: 20-25 mg of Amorphous Compound 1 di(hydrogen sulfate) was added to 24×1.5 mL screw cap vials. Into each vial, 100 μL of the appropriate solvent system was added at 25° C. to form solutions. Where complete dissolution was not observed, an additional 400 μL of solvent was added and the samples were stirred at 50° C. to allow further dissolution. The mother liquors of these samples were needle-filtered into a new set of vials. To all the solutions, anti-solvent addition was carried out at 25° C. Where sufficient precipitation was observed post-ASA, these were isolated and analyzed by XRPD. Where thin slurries or clear solutions were observed post-ASA, these were cooled to 5° C. at 0.1° C./min and stirred at 5° C. for ca 18 h. Any solids present were isolated and analyzed by XRPD. Table 37 below shows the solvent and anti-solvent for each experiment.

TABLE 37
Solvent and Anti-solvents used in
Anti-solvent Addition Experiments

| Solvent | Anti-Solvent |
|---|---|
| 1,4-Dioxane | tBME |
| 1-Propanol | tBME |
| 2-Methyl THF | tBME |
| 2-Propanol | tBME |
| 2-Propanol:water (50:50% v/v) | IPA |
| 2-Propanol:water (75:25% v/v) | IPA |
| Acetone | tBME |
| Acetone:water (75:25% v/v) | Acetone |
| Acetonitrile | tBME |
| Acetonitrile:water (75:25% v/v) | ACN |
| Dichloromethane (DCM) | tBME |
| Dimethylsulfoxide (DMSO) | Ethyl acetate |
| Ethanol | tBME |
| Ethanol:water (50:50% v/v) | Ethanol |
| Ethanol:water (90:10% v/v) | Ethanol |
| Solvent | Anti-Solvent |
| Ethyl Acetate | tBME |
| Isopropyl acetate | tBME |
| Methanol | tBME |
| Methylethyl Ketone (MEK) | tBME |
| Methylisobutyl Ketone (MiBK) | tBME |
| N,N′-Dimethylformamide (DMF | tBME |
| Tetrahydrofuran (THF | tBME |
| Toluene | tBME |
| Water | Acetone |

Reactive Crystallization: 15-20 mg of Compound 1 was added to 18×1.5 mL screw cap vials. Into each vial, 100 μL of the appropriate solvent system was added at 40° C. 2.05 mole equiv. of H₂SO₄ was also added to the vials as solutions in 100 μL of the respective solvent systems. The samples were stirred at 40° C. for another approximately 30 mins, cooled to 5° C. at 0.1° C./min and left stirring at 5° C. for about 18 h. At 5° C., solids present in any samples were isolated and analyzed by XRPD. For samples that were clear solutions at 5° C., anti-solvent addition was carried out at 5° C. to encourage crystallization. Any solids present were isolated and analyzed by XRPD.

Drying Studies: The XRPD plate containing the damp samples from each of the above experiments was placed in an oven to dry under vacuum at ambient temperature for 48-72 hours. Post-drying, the dried samples were then re-analyzed by XRPD to check the solid form.

Results: Pre-drying results are set forth in Table 38 with post-drying results in Table 39. Results were determined by X-ray powder diffraction in accordance with XRPD Method A.

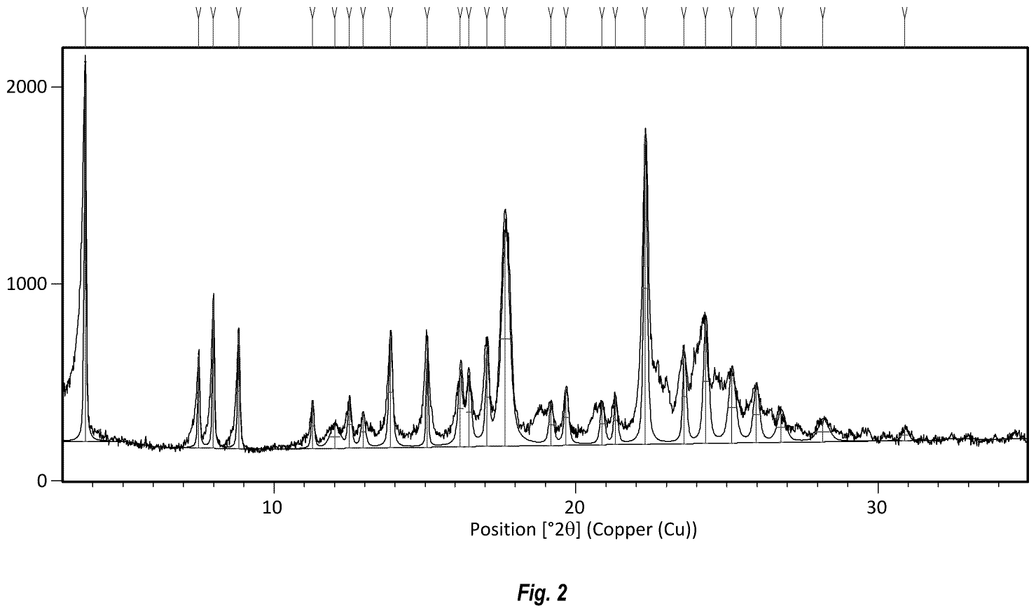

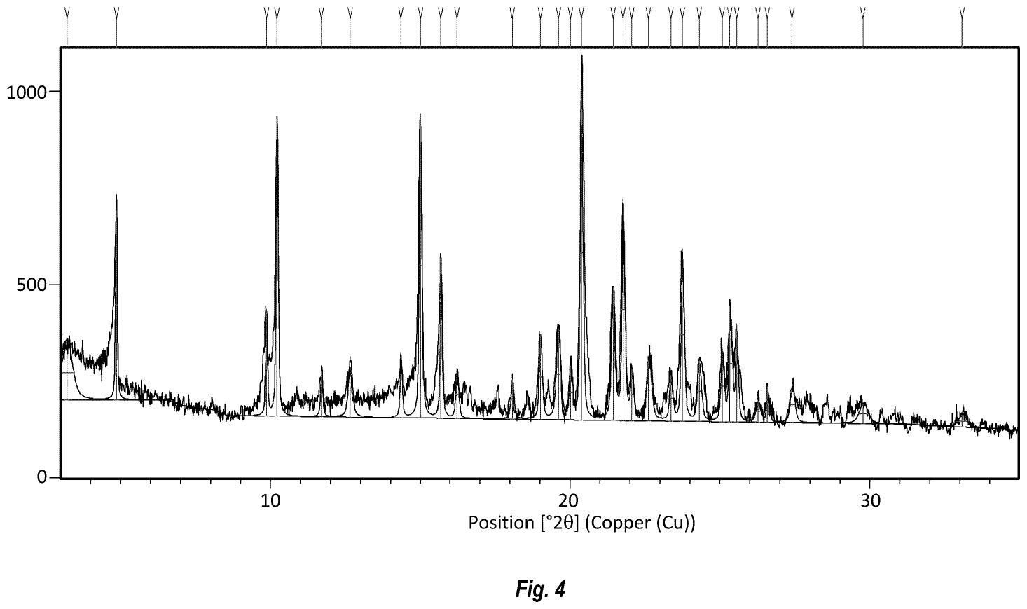

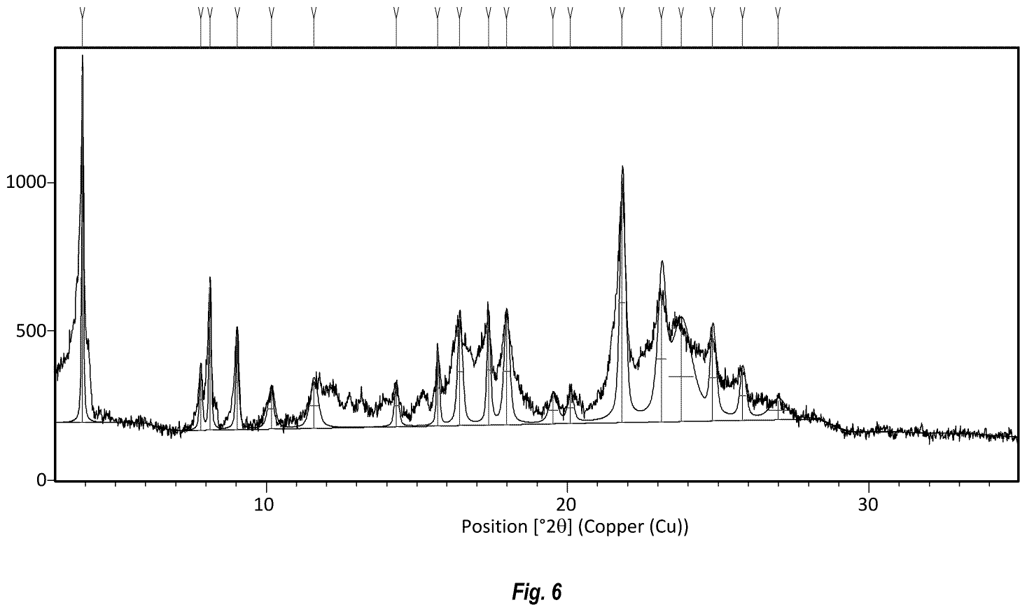

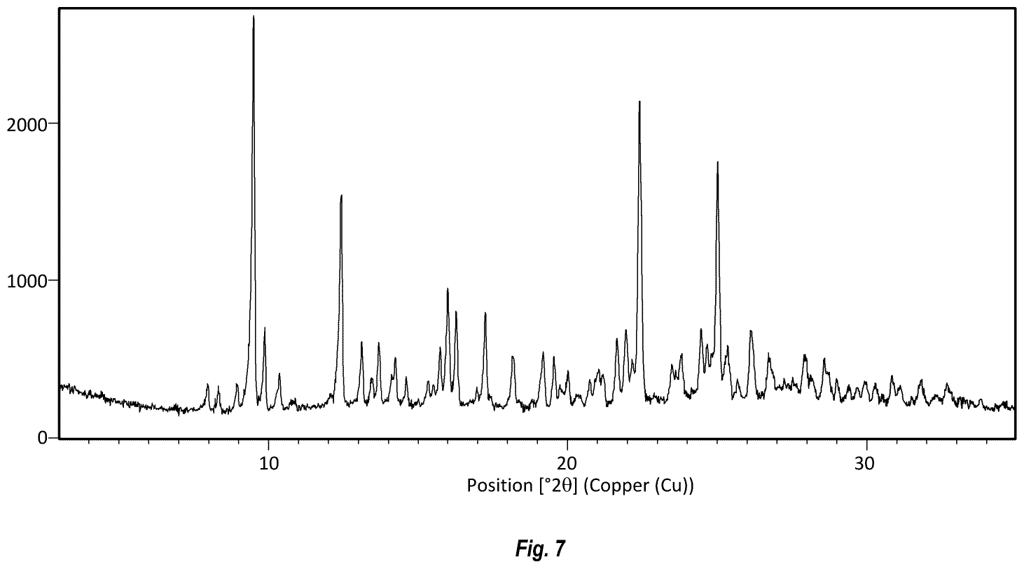

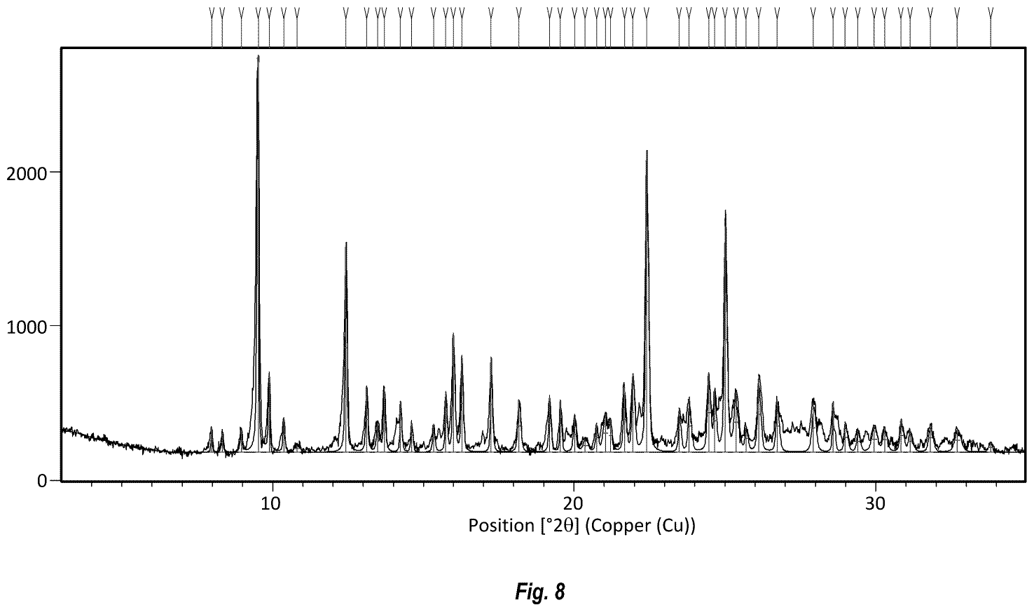

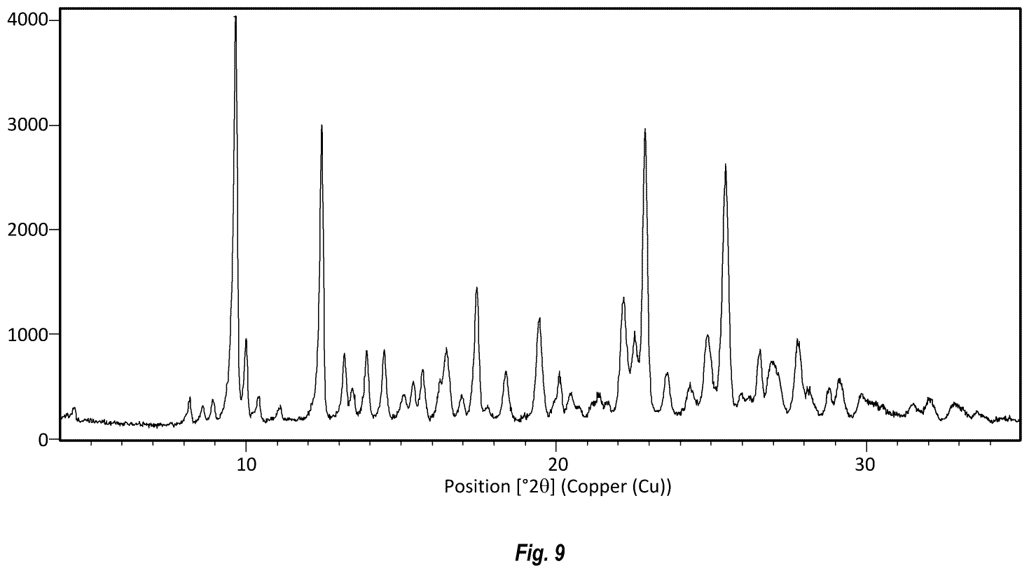

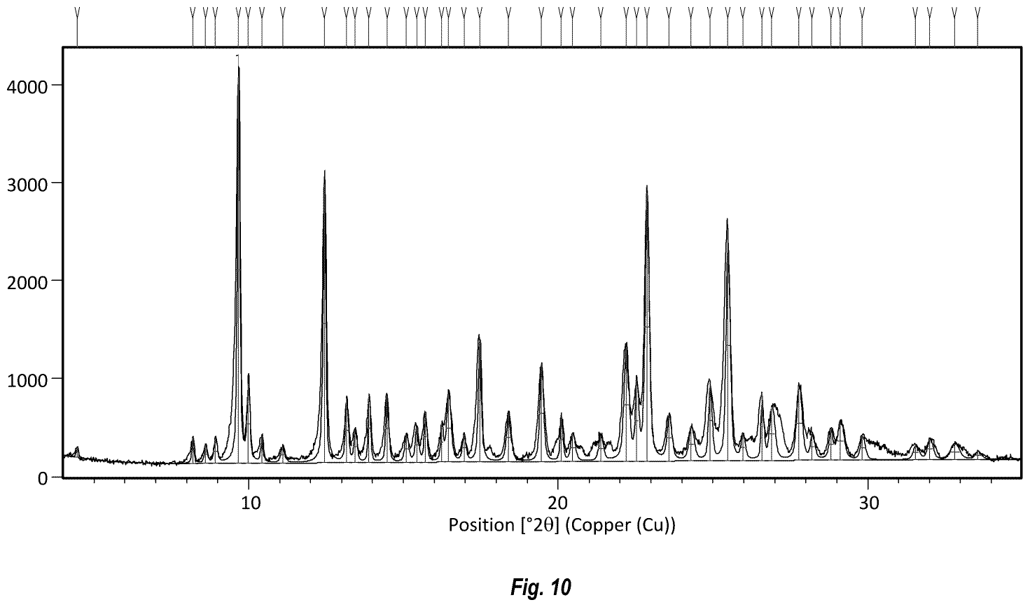

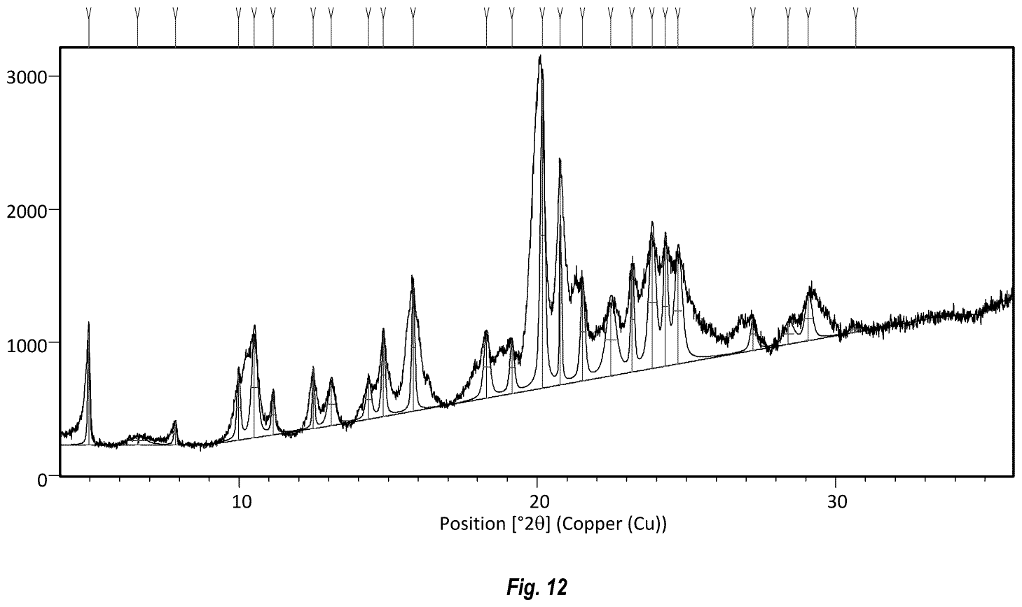

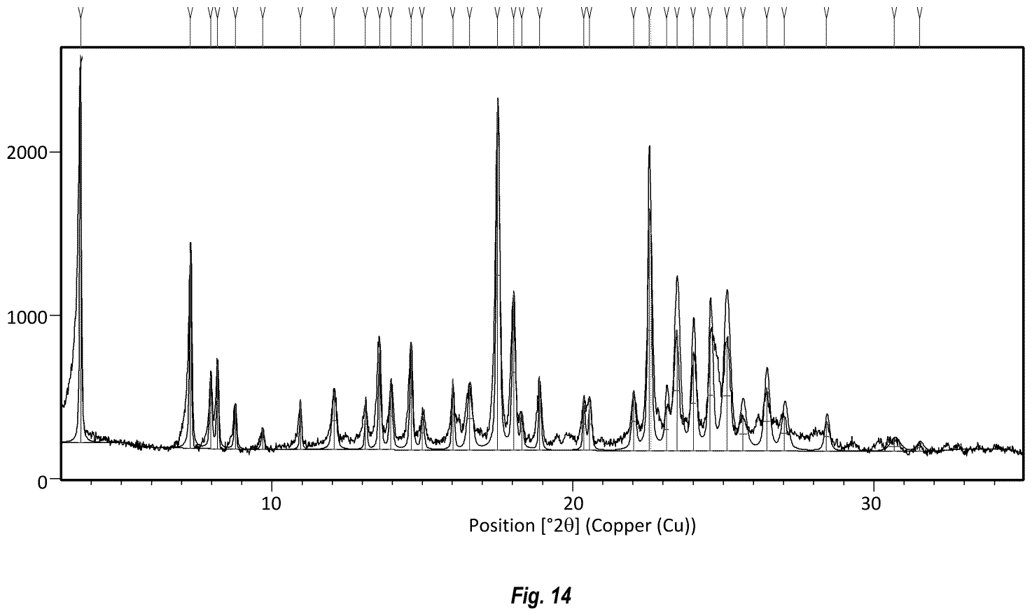

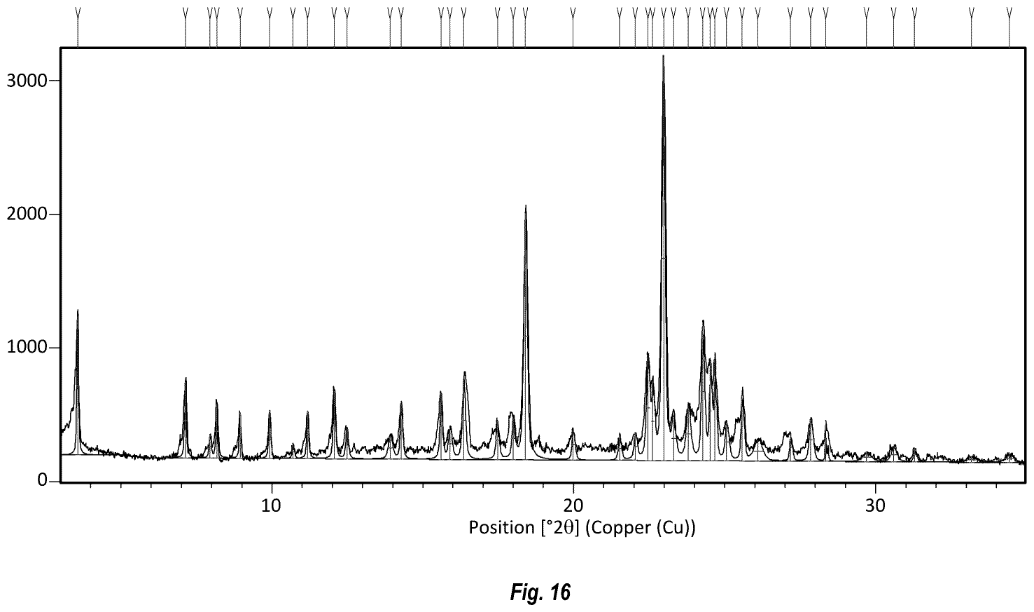

FIGS. 1-6 and 11-16 are examples of X-ray powder diffraction patterns for crystalline Compound 1 di(hydrogen sulfate) Forms 1-3 and Forms 5-7 including patterns whose peaks are picked. FIGS. 7-10 correspond to crystalline Compound 1 mono(hydrogen sulfate) Form 4.

TABLE 38
Results obtained from the solid form screen - XRPD (Damp)

| | XRPD (Damp) | | | | | |
|---|---|---|---|---|---|---|
| | Temp | | | Anti- | Solvent | Re- |
| | cy- | Evapo- | Crash | solvent | drop | active |
| Solvent System | cling | ration | cooling | addition | grinding | cryst. |
| 1,4-Dioxane | 2, PC | 2, PC | ns | Am | Am | 2 |
| 1-Propanol | Am | 2 | ins. | 2, PC | 2, PC | 2 |
| 2-Methyl THF | Am | 2, PC | ns | Am | Am | 2 + Am |
| 2-Propanol | Am | 2, PC | ins. | Am | Am | Am |
| 2-propanol:water | 2 | 2, PC | 2 | 2 | 2 | 2 |
| (50:50% v/v) | | | | | | |
| 2-propanol:water | 2 | 2 | 2, PO | 2 | 2 | 2, PC |
| (75:25% v/v) | | | | | | |
| Acetone | 2, PC | 2, PC | ns | Am | Am | 2 |
| Acetone:water | 2 | 2 | 2 | 2 | 2 | 2 |
| (75:25% v/v) | | | | | | |
| Acetonitrile | 6 | 6, PC | ns | ins. | 1 | 6 |
| Acetonitrile:water | 2 | NCO | 7 | 2 | 2 | 7 |
| (75:25% v/v) | | | | | | |
| DCM | 3 | 2 + 6 | ns | ins. | Am | NCO |
| DMSO | ns | ns. | ns | Am | Am | NCO |
| Ethanol | 2 | 2 | ins. | 2 | 2 | 2 |
| Ethanol:water | 2 | 2 | ns | 2 | 2 | 2 |
| (50:50% v/v) | | | | | | |
| Ethanol:water | 2 | 2 | 2 | 2 | 2 | 2 |
| (90:10% v/v) | | | | | | |
| Ethyl Acetate | Am | ins. | ns | ns | Am | Am |
| Isopropyl Acetate | Am | ns | ns | ns | Am | NCO |
| Methanol | Am | ns | 2 + Am | 2 | 2, PC | Am |
| MEK | 2, PC | ins. | ns | ns | Am | 6+ |
| MiBK | Am | ns. | ns | ns | Am | Am |
| DMF | ns. | ns. | ns | Am | Am | NCO |
| THF | M4+ | 2, PC | ins | ins. | Am | NCO |
| Toluene | Am | ns. | ins | ns | Am | NCO |
| Water | 2 | 2 | ns | 2 | 2 | ns |

| | | | |
|---|---|---|---|
| 1 | Crystalline Form 1 | NCO | Not carried out |
| | di(hydrogen sulfate) | | |
| 2 | Crystalline Form 2 | PC | Poorly crystalline |
| | di(hydrogen sulfate) | | |
| | trihydrate | | |
| 3 | Crystalline Form 3 | ins | Insufficient solid |
| | di(hydrogen sulfate) | | |
| M4 | Crystalline Form 4 | ns | No solid |
| | mono(hydrogen sulfate) | | |
| 6 | Crystalline Form 6 | + | Extra peaks |
| | di(hydrogen sulfate) | | |
| 7 | Crystalline Form 7 | PO | Preferred orientation |
| | di(hydrogen sulfate) | | |
| Am | Amorphous | 2 + 6 | Crystalline Form 2 |
| | di(hydrogen sulfate) | | di(hydrogen sulfate) |
| | | | trihydrate and Crystalline |
| | | | Form 6 di(hydrogen sulfate) |
| 2 + Am | Crystalline Form 2 | | |
| | di(hydrogen sulfate) | | |
| | trihydrate and | | |
| | amorphous di(hydrogen | | |
| | sulfate) | | |

TABLE 39
Results obtained from the solid form screen - XRPD (Dried)

| | XRPD (Dried) | | | | | |
|---|---|---|---|---|---|---|
| | Temp | | | Anti- | Solvent | Re- |
| | cy- | Evapo- | Crash | solvent | drop | active |
| Solvent System | cling | ration | cooling | addition | grinding | cryst. |
| 1,4-Dioxane | 2, PC | 2, PC | ns | Am | Am | 2 |
| 1-Propanol | Am | 2 | ins. | 2, PC | 2 | 2 |
| 2-Methyl THF | Am | 2, PC | ns | Am | Am | 2 |
| 2-Propanol | Am | 2 | ins | Am | 2 | Am |
| 2-propanol:water | 2 | 2, PC | 2 | 2 | 2 | 2 |
| (50:50% v/v) | | | | | | |
| 2-propanol:water | 2 | 2 | 2, PO | 2 | 2 | 2 |
| (75:25% v/v) | | | | | | |
| Acetone | 2, PC | 2, PC | ns | Am | Am | 2 |
| Acetone:water | 2 | 2 | 2 | 2 | 2 | 2 |
| (75:25% v/v) | | | | | | |
| Acetonitrile | 3 | 6+, PC | ns | ins. | 3 | 1+ |
| Acetonitrile:water | 2 | NCO | 1 + | 2 | 2 | 3+ |
| (75:25% v/v) | | | 2 + 7 | | | |
| DCM | 3 | 2 + 3 | ns | ins. | Am | NCO |
| DMSO | ns. | ns | ns | Am | Am | NCO |
| Ethanol | 2 | 2 | ins | 2 | 2 | 2 |
| Ethanol:water | 2 | 2 | ns | 2 | 2 | 2 |
| (50:50% v/v) | | | | | | |
| Ethanol:water | 2 | 2, PC | 2 | 2 | 2 | 2 |
| (90:10% v/v) | | | | | | |
| Ethyl Acetate | Am | ins. | ns | ns | Am | Am |
| Isopropyl Acetate | Am | ns | ns | ns | Am | NCO |
| Methanol | Am | ns | 2 | 2 | 2, PC | Am |
| MEK | 2, PC | ins. | ns | ns | Am | 1 |
| MiBK | Am | ns. | ns | ns | Am | Am |
| DMF | ns. | ns. | ns | Am | Am | NCO |
| THF | M4+ | 2, PC | ins | ins. | Am | NCO |
| Toluene | Am | ns. | ns | ns | Am | NCO |
| Water | 2 | 2 | ns | 2 | 2 | ns |

| | | | |
|---|---|---|---|
| 1 | Crystalline Form 1 | NCO | Not carried out |
| | di(hydrogen sulfate) | | |
| 2 | Crystalline Form 2 | PC | Poorly crystalline |
| | di(hydrogen sulfate) | | |
| | trihydrate | | |
| 3 | Crystalline Form 3 | ins | Insufficient solid |
| | di(hydrogen sulfate) | | |
| M4 | Crystalline Form 4 | ns | No solid |
| | mono(hydrogen sulfate) | | |
| 6 | Crystalline Form 6 | PO | Preferred orientation |
| | di(hydrogen sulfate) | | |
| 2 + 3 | Crystalline Form 2 | 1 + 2 + 7 | Crystalline Form 1 |
| | di(hydrogen sulfate) | | di(hydrogen sulfate), |
| | trihydrate and | | Crystalline Form 2 |
| | Crystalline Form 3 | | di(hydrogen sulfate) |
| | di(hydrogen sulfate) | | trihydrate, and Crystalline |
| | | | Form 7 di(hydrogen sulfate) |

Example 2A-Preparation of Crystalline Form 3 Compound 1 di(hydrogen sulfate): Into a 20 mL vial containing ca. 500 mg of crystalline Form 2 Compound 1 di(hydrogen sulfate) trihydrate, 5 mL of acetonitrile was added. The resultant slurry sample was slurried at 5° C. for 1-4 days. The solid was isolated via vacuum filtration and analyzed by XRPD. The solid sample was then dried under reduced pressure at 40° C. for 2-3 days.

XRPD analysis of the damp sample was consistent with crystalline Form 6 Compound 1 di(hydrogen sulfate) and conversion to crystalline Form 3 Compound 1 di(hydrogen sulfate) was observed upon drying under reduced pressure at 40° C. crystalline Form 3 Compound 1 di(hydrogen sulfate) showed partial crystallinity after drying.

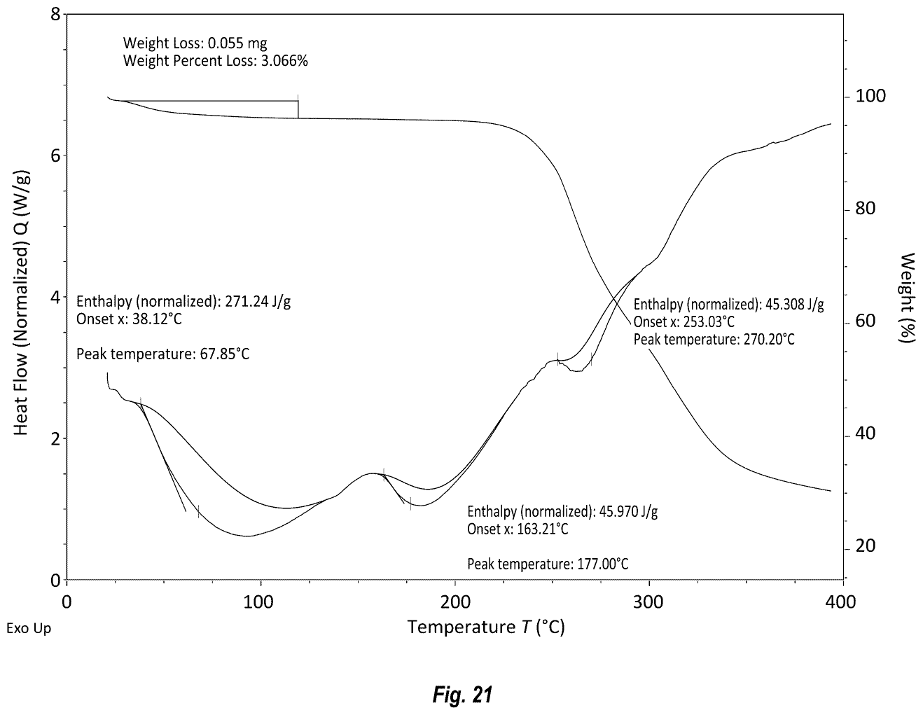

TG/DSC data of crystalline Form 3 Compound 1 di(hydrogen sulfate) showed a mass loss of 3.1% between 20-120° C., which corresponded with an endothermic event at peak onset 38° C. (peak at 68° C.) (FIG. 21). This is consistent with a water loss of about 1.5 mole equivalent of water (3.1% being 1.47 mole equivalents). A small endothermic event was observed at an onset 163° C. (FIG. 21) which is likely melting.

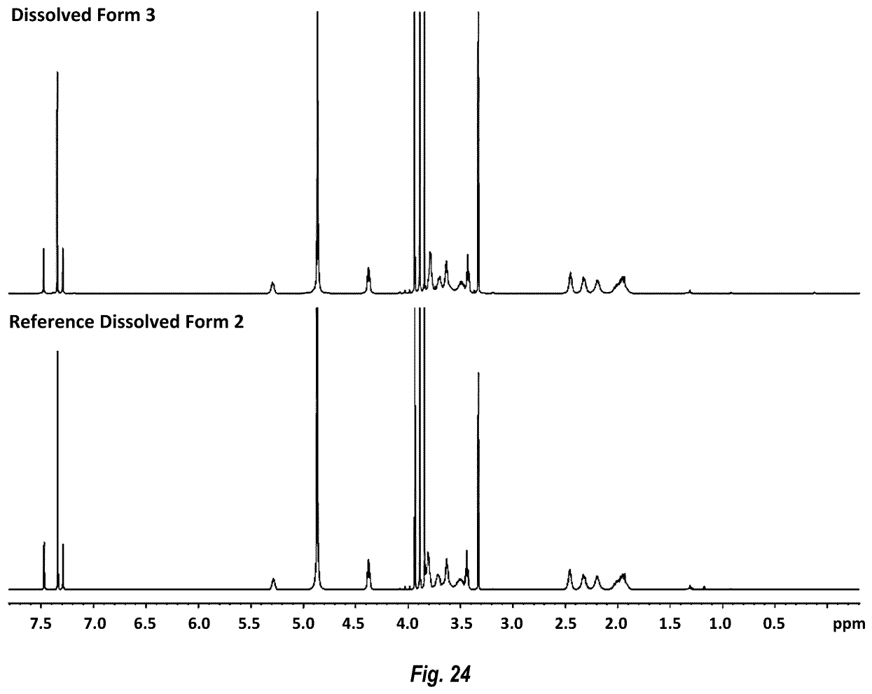

A DSC analysis showed a broad endothermic event at onset 47° C. (FIG. 22), which is likely due to dehydration. KF analysis indicated an average moisture content of 1.89% w/w. An FT-IR spectrum was similar to the reference spectrum of crystalline Form 2 Compound 1 di(hydrogen sulfate) trihydrate, although there are peaks not present in the OH stretching region (around 3500 cm⁻¹) in the crystalline Form 3 Compound 1 di(hydrogen sulfate) spectrum that are present in the crystalline Form 2 Compound 1 di(hydrogen sulfate) trihydrate spectrum (FIGS. 23A and 23B). A ¹H-NMR spectrum was similar to the ¹H-NMR spectrum of crystalline Form 2 Compound 1 di(hydrogen sulfate) trihydrate, and no residual solvent (acetonitrile) was observed (FIG. 24), noting, however, that ¹H-NMR does not provide solid-state structural information.

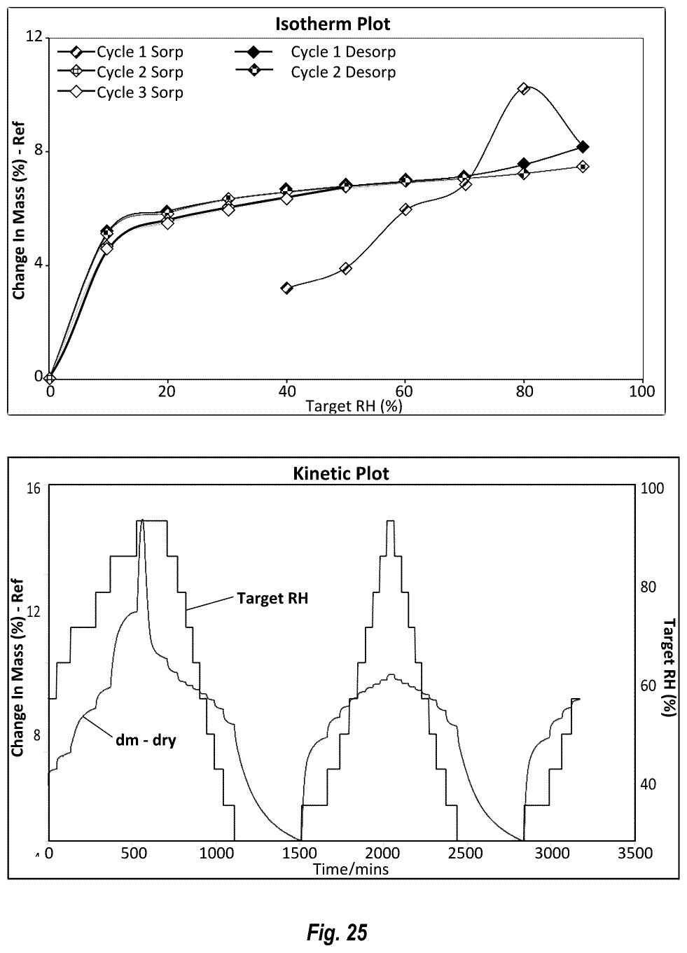

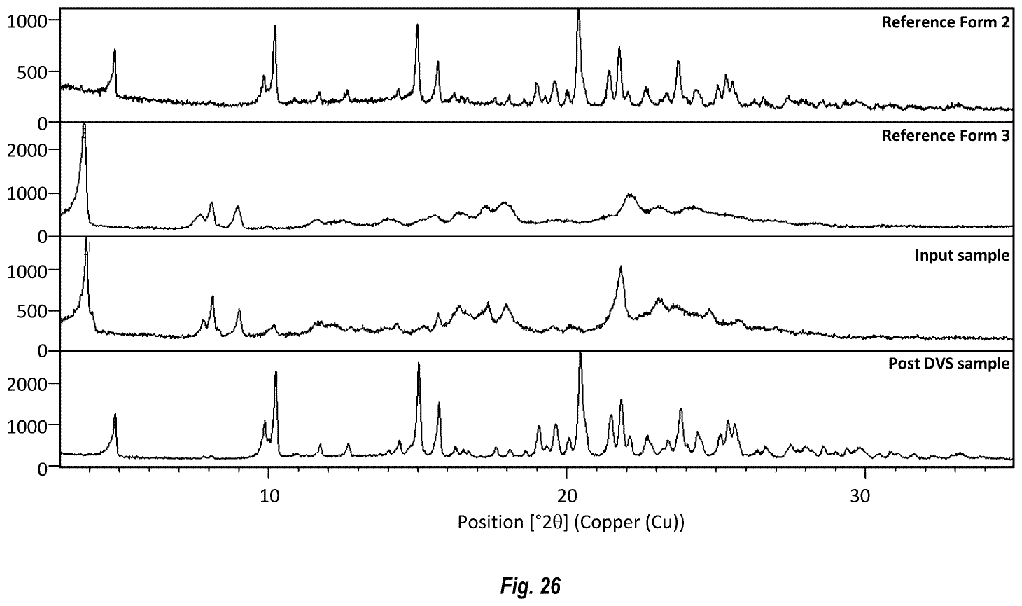

DVS analysis showed a moisture uptake of ca. 7.1% between 40-80% RH during the first sorption cycle. Possible recrystallization/form change was observed between 80-90% RH steep uptake of about 5.2% was observed between 0-10% RH during the second and third sorption cycles. A more gradual uptake of ca. 2.1% was observed between 10-80% RH during the second sorption cycle. No significant hysteresis was observed (not taking into account the first sorption cycle) (FIG. 25). XRPD analysis of the post-DVS sample showed conversion to crystalline Form 2 Compound 1 di(hydrogen sulfate) trihydrate (FIG. 26). HPLC analysis indicated a solid purity of 99.83% area.

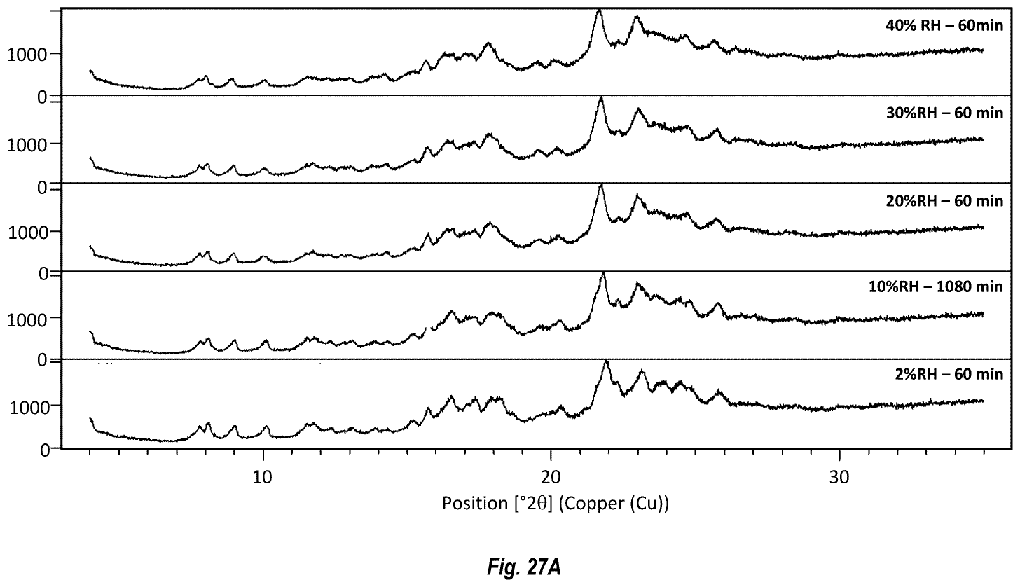

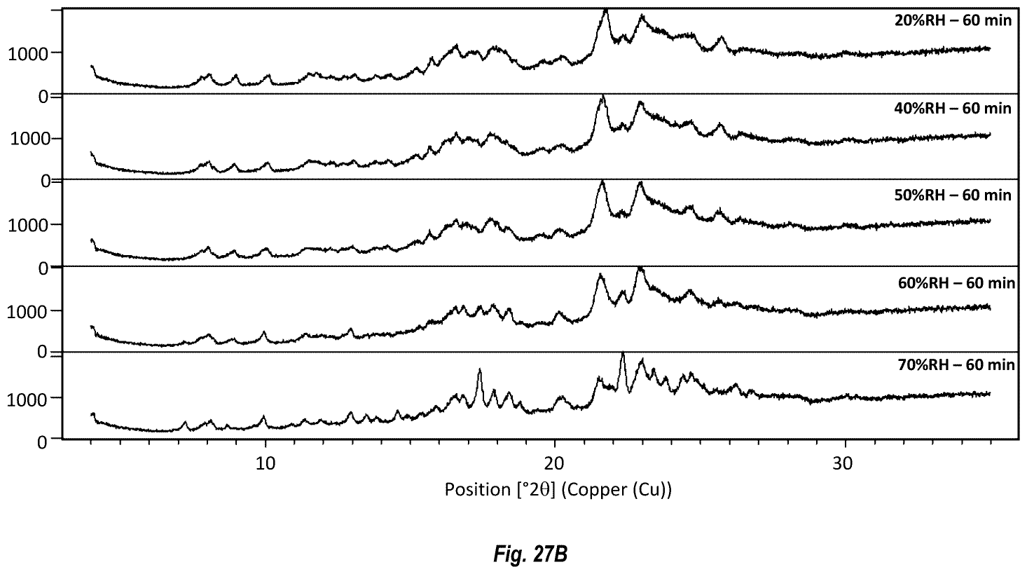

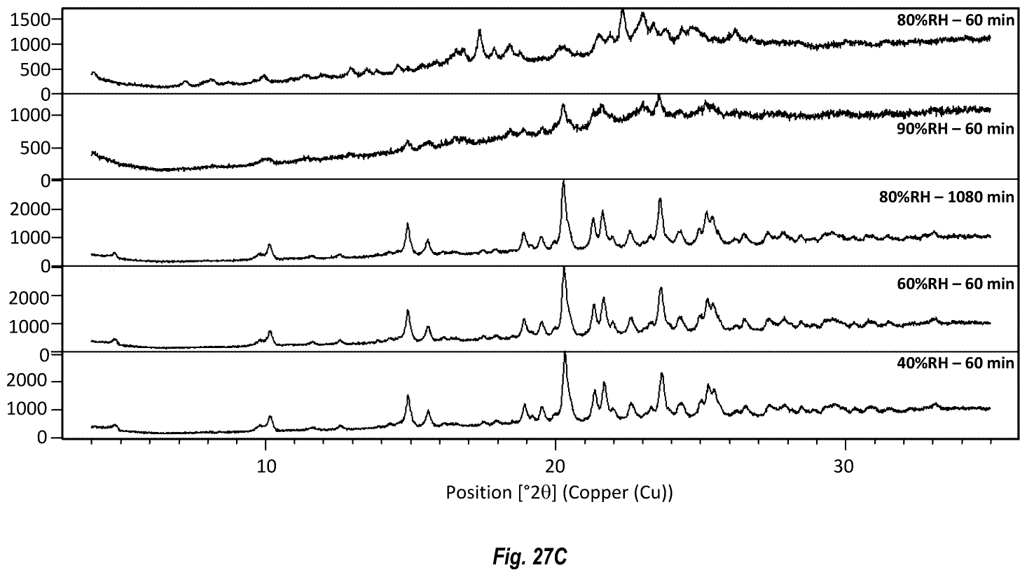

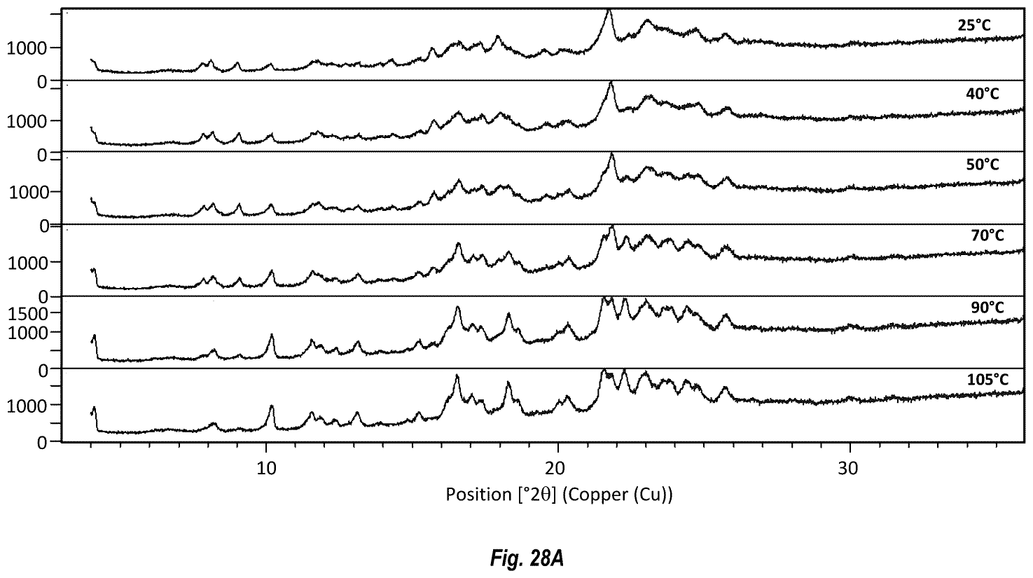

VH-XRPD analysis carried out between 2-90% RH showed crystalline Form 3 Compound 1 di(hydrogen sulfate) being retained between 2-60% RH. Peaks corresponding to crystalline Form 6 Compound 1 di(hydrogen sulfate) were observed (in addition to crystalline Form 3 Compound 1 di(hydrogen sulfate)) between 70-80% RH. Conversion to crystalline Form 2 Compound 1 di(hydrogen sulfate) trihydrate was observed after extended exposure to ≥80% RH, and this was maintained during final desorption cycle to 40% RH (FIGS. 27A, 27B, and 27C). The results are summarized in Table 40.

TABLE 40
Results of VH-XRPD analysis for Crystalline
Form 3 Compound 1 di(hydrogen sulfate)

| | Rel. Humidity | Hold time | XRPD |
|---|---|---|---|
| Cycle | (% RH) | (min) | Results |
| Desorption | 40 | 60* | 3 |
| | 30 | 60 | 3 |
| | 20 | 60 | 3 |
| | 10 | 1080 | 3 |
| | 2 | 60 | 3 |
| Absorption | 20 | 60 | 3 |
| | 40 | 60 | 3 |
| | 50 | 60 | 3 |
| | 60 | 60 | 3 |

| | | | |
|---|---|---|---|
| 70 | 60 | 3 | 6 |
| 80 | 60 | 3 | 6 |
| 90 | 60 | 3 | 2 |

| | | | |
|---|---|---|---|
| Desorption | 80 | 1080 | 2 |
| | 60 | 60 | 2 |
| | 40 | 60 | 2 |

*XRPD scan for this sample was taken after 40 min (i.e. 20 mins before the end of the hold period)
2 Crystalline Form 2 di(hydrogen sulfate) trihydrate
3 Crystalline Form 3 di(hydrogen sulfate)
6 Crystalline Form 6 di(hydrogen sulfate)

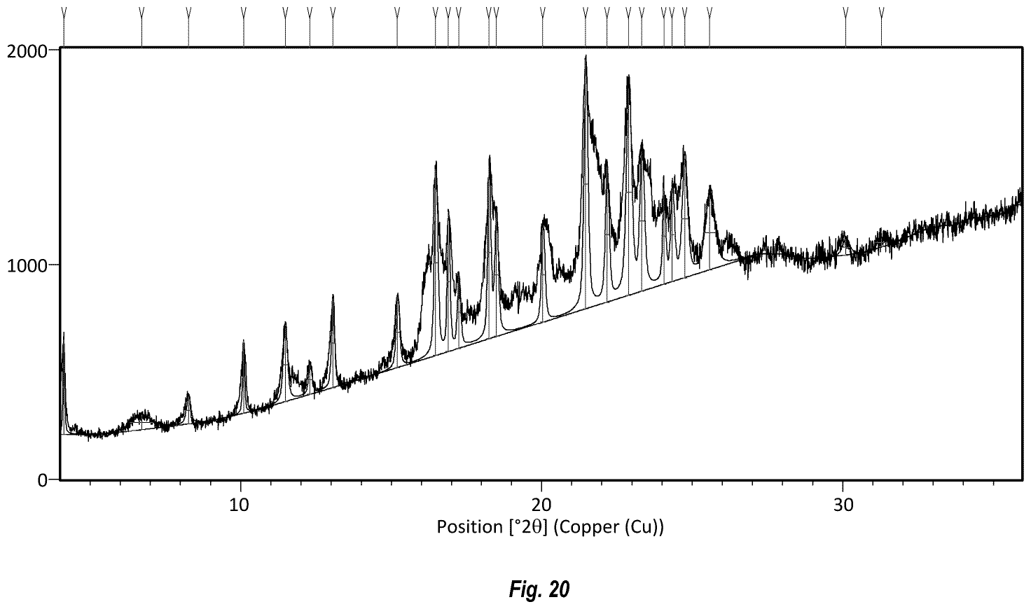

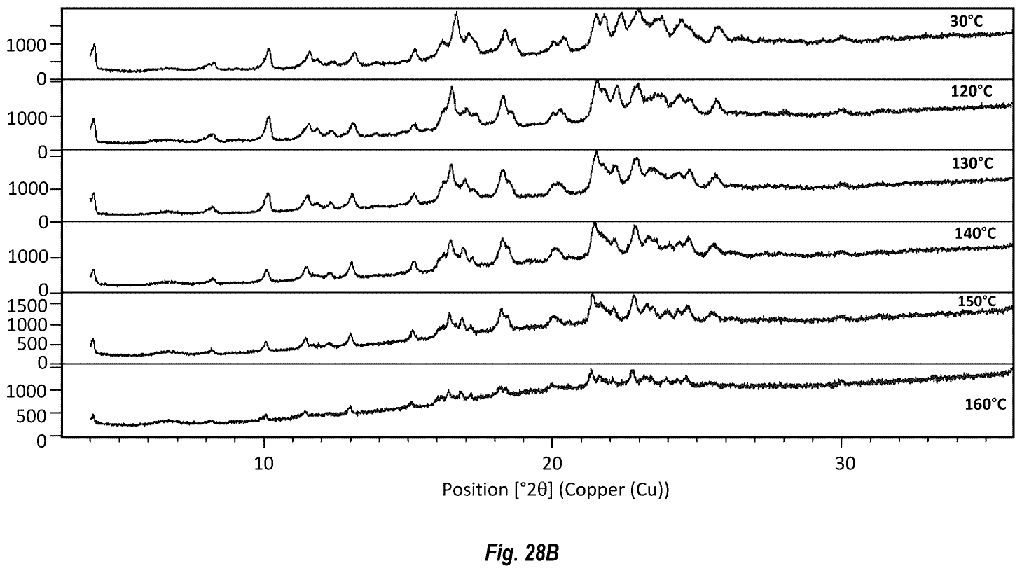

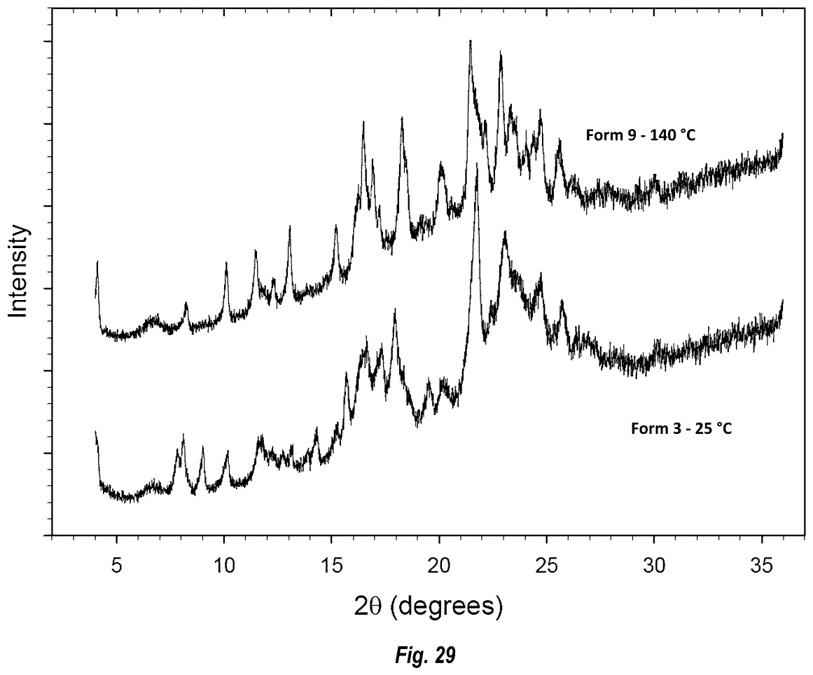

VT-XRPD analysis carried out between 25-160° C. showed crystalline Form 3 Compound 1 di(hydrogen sulfate) being retained between 25-40° C. Peaks (corresponding to crystalline Form 9 Compound 1 di(hydrogen sulfate)) were observed to form between 50° C.-160° C. FIGS. 28A and 28B are a series of variable-temperature XRPD patterns ranging from 25° C. to 160° C. and FIG. 29 is an overlay showing crystalline Form 3 Compound 1 di(hydrogen sulfate) at 25° C. and crystalline Form 9 Compound 1 di(hydrogen sulfate) at 140° C. An exemplary XRPD pattern of crystalline Form 9 Compound 1 di(hydrogen sulfate) is shown in FIGS. 19 and 20. Reversion to crystalline Form 3 Compound 1 di(hydrogen sulfate) was not observed during cooling. Reduced crystallinity was observed at 160° C. which is likely due to the onset of melting. The results are summarized in Table 41.

TABLE 41
Results of VT-XRPD analysis for Crystalline
Form 3 Compound 1 di(hydrogen sulfate)

| | Temp | Hold time | XRPD |
|---|---|---|---|
| Cycle | (° C.) | (min) | Results |
| Heating | 25 | 0 | 3 |
| | 40 | 30 | 3 |
| | 50 | 30 | 3 (trace of 9) |

| | | | | |
|---|---|---|---|---|
| | 70 | 30 | 3 | 9 |
| | 90 | 30 | 3 | 9 |
| | 105 | 30 | 3 | 9 |
| Cooling | 30 | 10 | 3 | 9 |
| Heating | 120 | 30 | 3 | 9 |
| | 130 | 30 | 3 | 9 |

| | | |
|---|---|---|
| 140 | 30 | 9 |
| 150 | 30 | 9 |

| | | | |
|---|---|---|---|
| 160 | 30 | 9 | Melt |

3 Crystalline Form 3 di(hydrogen sulfate)
9 Crystalline Form 9 di(hydrogen sulfate)
Melt Melt/Amorphous

The obtained data suggested that crystalline Form 3 Compound 1 di(hydrogen sulfate) is likely to be a partial or lower hydrated form of di(hydrogen sulfate) compared with crystalline Form 2 Compound 1 di(hydrogen sulfate) trihydrate.

Example 2B—7 day Stability Study on Crystalline Form 3 Compound 1 di(hydrogen sulfate) and Crystalline Form 4 Compound 1 mono(hydrogen sulfate): A seven-day study was conducted on samples of crystalline Form 3 Compound 1 di(hydrogen sulfate) and crystalline Form 4 Compound 1 mono(hydrogen sulfate) in open vials. Crystalline Form 3 Compound 1 di(hydrogen sulfate) could prepared according to Example 1 or 2A above. Crystalline Form 4 Compound 1 mono(hydrogen sulfate) could be prepared according to Example 14 below.

In the experiment, one set of vials was exposed to ambient light, temperature and humidity conditions. Another was exposed openly to 40° C. and 75% RH conditions and a third was sealed and exposed to 80° C. conditions. After 7 days, XRPD and HPLC measurements were collected. The results are summarized in Table 42 below.

TABLE 42
Results from the stability studies of Crystalline
Form 3 Compound 1 di(hydrogen sulfate) and Crystalline
Form 4 Compound 1 mono(hydrogen sulfate)

| Input Material | Conditions | XRPD | | HPLC (% area) |
|---|---|---|---|---|
| Form 3 | Input sample | 3 | | 99.83 |
| di(hydrogen | Ambient | 6 | | 99.84 |
| sulfate) | 40° C./75% RH | 2 | | 99.82 |
| | 80° C. | 3 | 9 | 99.80 |

| | | | |
|---|---|---|---|
| Form 4 | Input sample | M4 | 99.66 |
| mono(hydrogen | Ambient | M4 | 99.89 |
| sulfate) | 40° C./75% RH | M4 | 99.87 |
| | 80° C. | Amorphous* | 99.64 |

*Predominantly
2 Crystalline Form 2 di(hydrogen sulfate) trihydrate
3 Crystalline Form 3 di(hydrogen sulfate)
M4 Crystalline Form 4 mono(hydrogen sulfate)
6 Crystalline Form 6 di(hydrogen sulfate)
9 Crystalline Form 9 di(hydrogen sulfate)

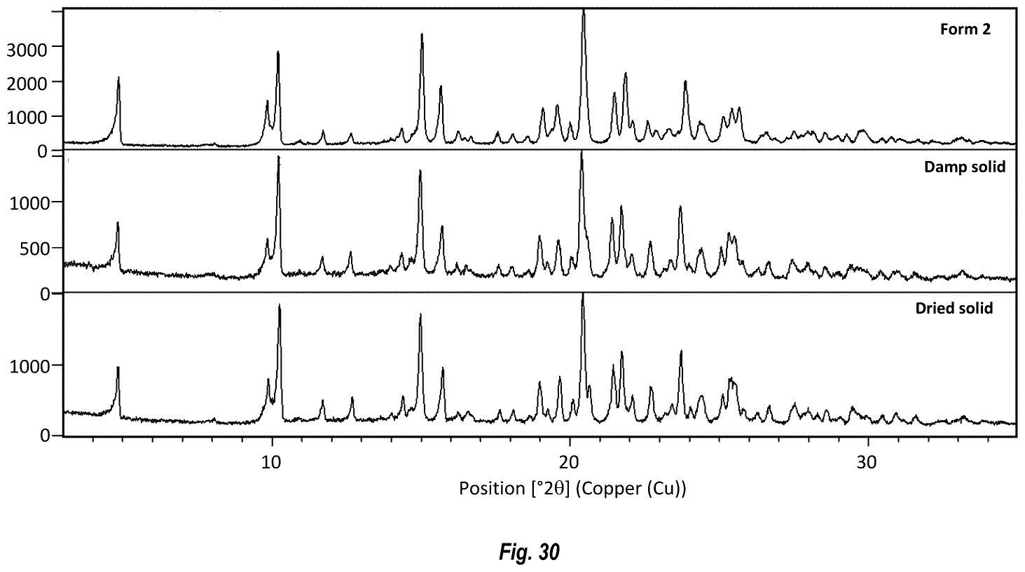

Example 3—Preparation of Crystalline Form 2 Compound 1 di(hydrogen sulfate) trihydrate: A sample of Compound 1 (free base) and 6 vol. of IPA/water (75:25% v/v) were added to a 20 mL scintillation vial. 2.05 mole equivalents of sulfuric acid were added as a solution in 2 vol. of IPA/water (75:35% v/v). The solution was stirred for about one hour at 40° C. The temperature was then cycled between 40° C. and 5° C. with a 0.1° C./min ramp and a one hour hold between each step. After about 48 hours of cycling, a clear solution was still observed. Up to 6 vol. of anti-solvent (tBME) was added to 40° C. to facilitate precipitation. The experiment (now a slurry) was further temperature cycled for 24 hours. The solids were then isolated via vacuum filtration and dried under vacuum at about 40° C. for 48 hours. The damp and dried solids were subsampled and analyzed by XRPD and shown to be crystalline Form 2 Compound 1 di(hydrogen sulfate) trihydrate as shown in FIG. 30. TG/DSC data show a mass loss of 7.3% corresponding to an endothermic event with an onset of 67° C. which is likely due to water loss (approximating 3.6 mole equivalents of water) (FIG. 31). DSC shows an endotherm at an onset of 144° C., which is consistent with a melting event (FIG. 32) whereas a small melting event was observed in the TG/DSC data with an onset of 130° C. Compared to the free base, crystalline Form 2 Compound 1 di(hydrogen sulfate) trihydrate begins to melt around 130° C. or 144° C. depending on the method used, whereas the crystalline Form 1 Compound 1 (free base) starts to melt at 65° C. (see Example 11 below). This large difference in melting points is significant for pharmaceutical development and processing, where higher melting solids are generally preferred.

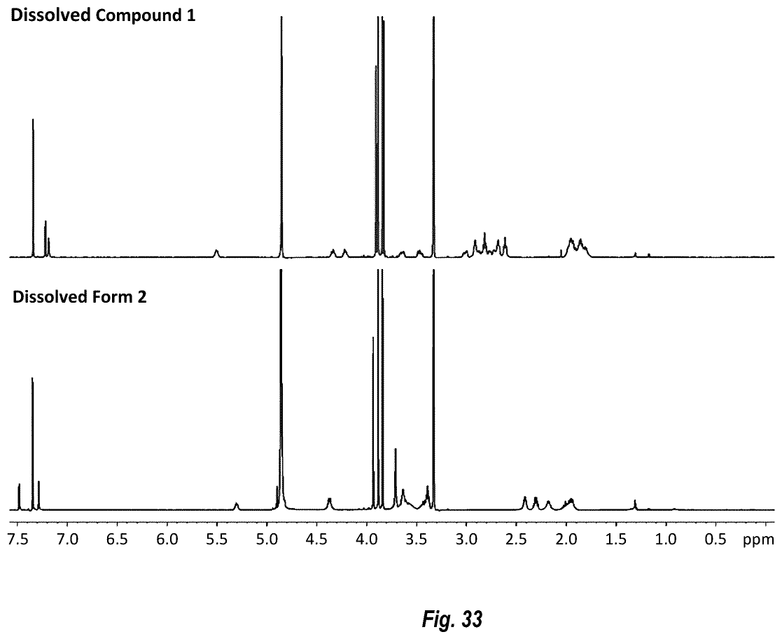

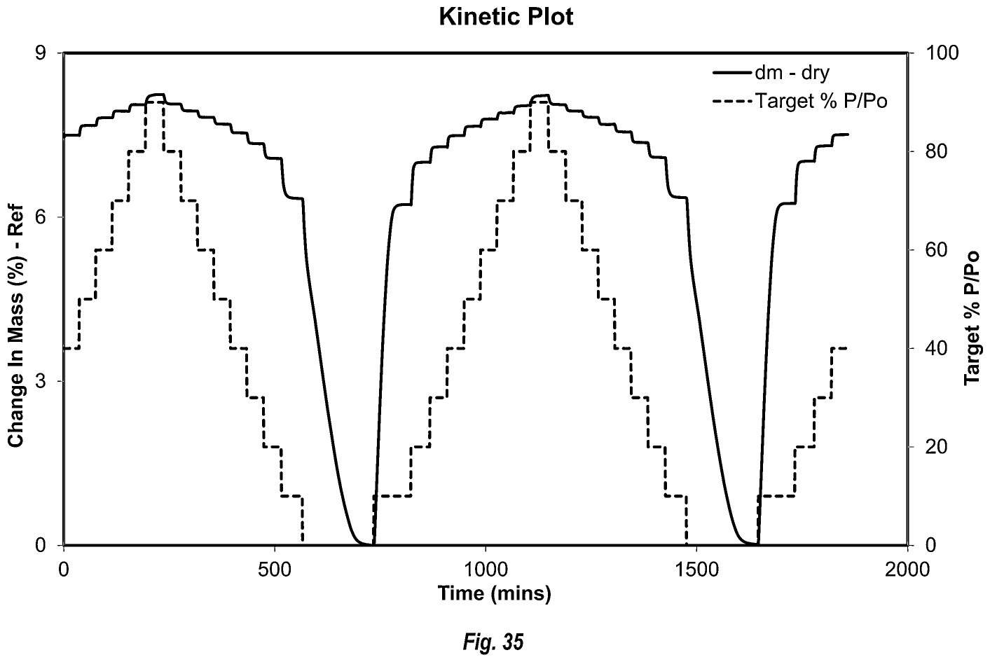

KF analysis shows a water content of 6.74%. ¹H-NMR is consistent with the Compound 1 (free base) structure with peak shifts indicating salt formation (FIG. 33). A DVS experiment (FIG. 34 and FIG. 35) shows a moisture uptake of about 6.3% between 0 and 10% RH with a more gradual uptake of 1.7% as RH is raised to 80% from 10%. As discussed in more detail in Example 9, a variable humidity XRPD experiment showed conversion to crystalline Form 5 Compound 1 di(hydrogen sulfate) at about 6% RH. In addition, as discussed in more detail in Example 9, a variable temperature XRPD analysis showed conversion to crystalline Form 5 Compound 1 di(hydrogen sulfate) at temperatures 80° C. to 130° C.

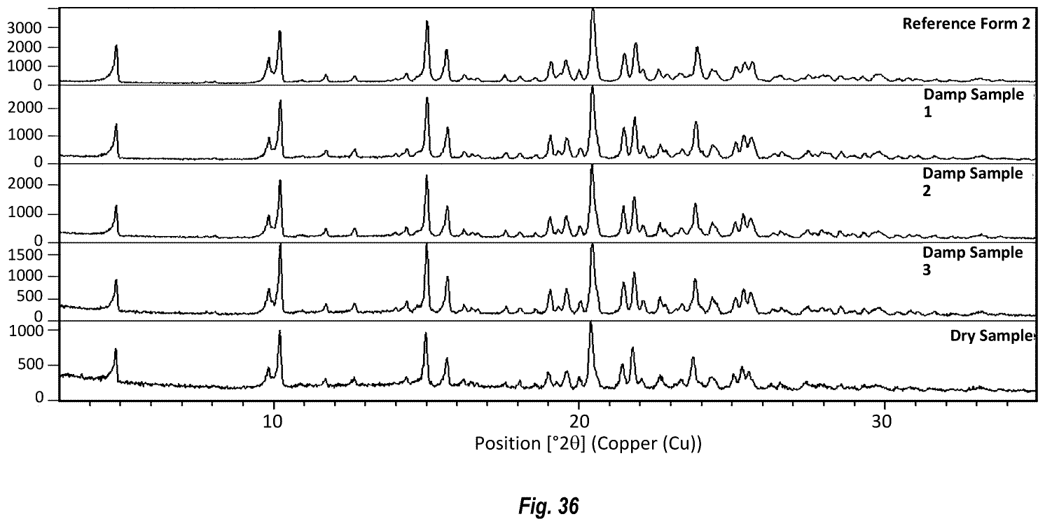

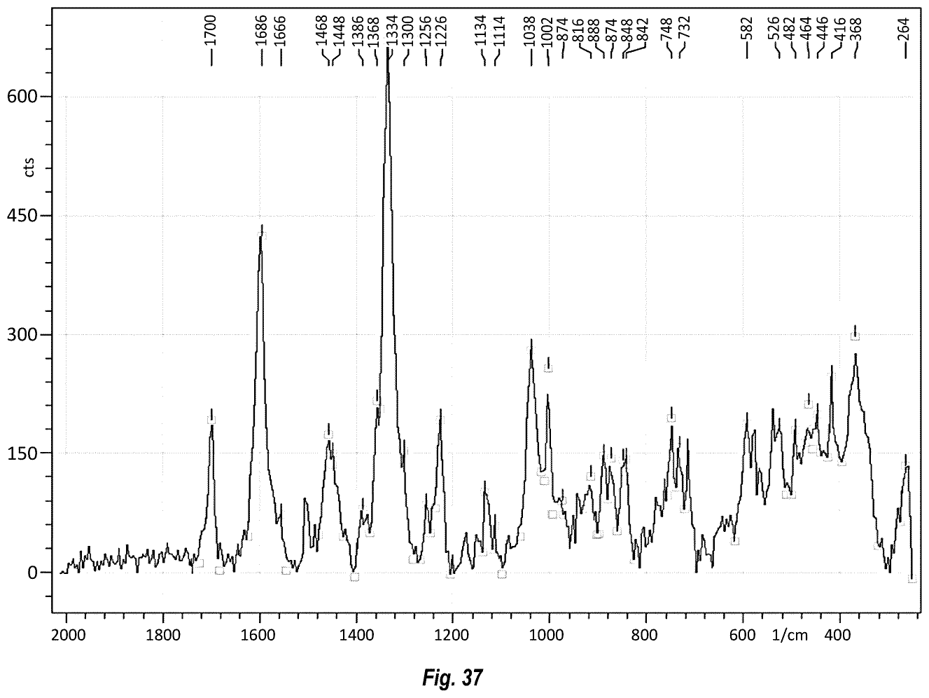

Example 4-Preparation of Crystalline Form 2 Compound 1 di(hydrogen sulfate) trihydrate: Approximately 1.5 g of Compound 1 was added to three 20 mL scintillation vials. 5 mL of 2-propanol:water (75:25% v/v) was added to each vial, and the samples were stirred at 40° C. 2.05 mole equiv. of H₂SO₄ was added to each sample as solutions in 5 mL of propanol:water (75:25% v/v). Clear solutions were observed in all samples. Stirring continued at 40° C. for about 1.5 hours after addition of H₂SO₄. tBME was then added to each sample, also at 40° C., as an anti-solvent in 1 mL aliquots until the solutions turned cloudy. The samples were then temperature-cycled between 40° C. and 5° C. for about 20 hours with 0.1° C./min ramp and a 1 hour hold between steps. The resultant slurries were subsampled, and solids isolated via centrifugation. The isolated solids were analyzed by XRPD. The remainder of the slurries were vacuum filtered as one sample using a Buchner funnel. The filter cake was dried under vacuum at 40° C. for 48 hours. 4.87 g of crystalline Form 2 Compound 1 di(hydrogen sulfate) trihydrate was obtained; isolated yield=81%. An XRPD pattern was collected confirming crystalline Form 2 Compound 1 di(hydrogen sulfate) trihydrate (FIG. 36). A Raman spectrum was also collected (FIG. 37).

Example 5—Preparation of Crystalline Form 2 Compound 1 di(hydrogen sulfate) trihydrate: 253.75 g of a mixture of crystalline Form 1 Compound 1 (free base) and crystalline Form 2 Compound 1 (free base) was added to a 5 L temperature-controlled reactor. 1.10 L of 1-propanol:water (80:20% v/v) was added to the reactor, followed by stirring at 50° C. at 100 RPM. 2.5 equivalents of sulfuric acid (95% wt.) were added to the reactor as a solution in 0.17 L of the solvent system, achieving a concentration of about 200 mg/mL. The experiment was equilibrated at 50° C., cooled to 40° C. and seeded with 1% crystalline Form 2 Compound 1 di(hydrogen sulfate) trihydrate. The seed crystal can be prepared methods such as those disclosed herein. Post-seeding, equilibration of ca. 6 hr was applied. The stirring speed was increased to 120 RPM during the equilibration period. The experiment was cooled to 20° C. at 0.2° C./min and equilibrated at 20° C. for 1 hour. At 20° C., anti-solvent addition with 1-propanol was carried out at 0.25 L/hr. 2.96 L of 1-propanol was added to reach a final ratio of 94:6% v/v. The stirring speed was increased to 190 RPM during the addition. Equilibration at 20° C. for about 1 hour was applied post-addition followed by cooling to 5° C. at 0.2° C./min and stirring at 5° C. for about 20 hours. The resulting slurry was vacuum filtered and the resulting isolated cake was washed with 0.5 L of the precooled resulting solvent system. The filter cake was dried under vacuum at 40° C. for 4 days. The dried solids were exposed to ambient conditions for about 20 hours to allow time for moisture equilibration. The moisture content of the solids after about 20 hours exposure to ambient condition was measured as 4.33% w/w. The solids were then re-exposed to ambient conditions for 2 days to allow time for further moisture uptake and equilibration during which time the solids were manually mixed intermittently.

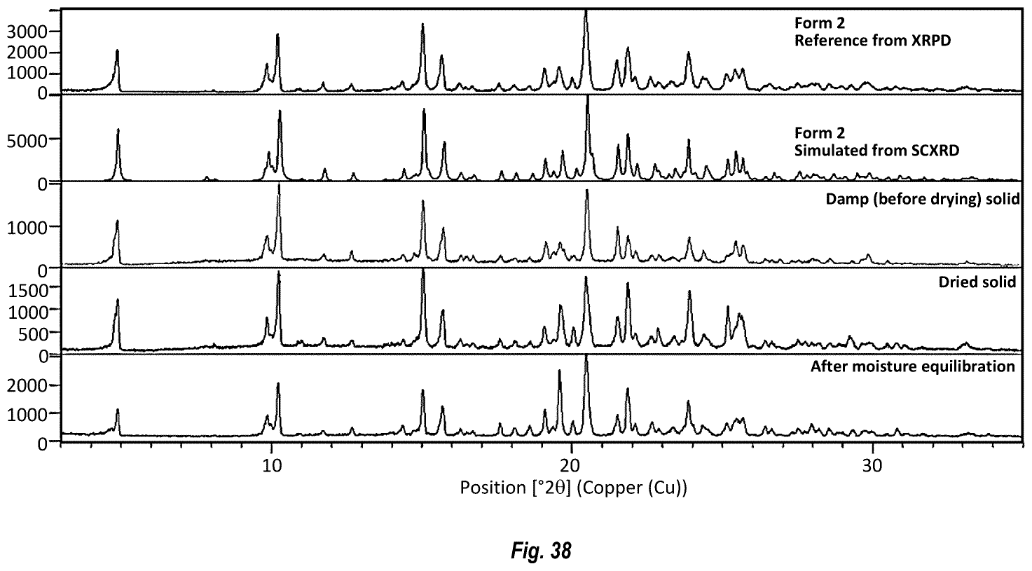

The resulting solids were shown to be crystalline Form 2 Compound 1 di(hydrogen sulfate) trihydrate by XRPD. FIG. 38 shows the material before drying, after drying, and after drying and moisture equilibration. All diffractograms are that of crystalline Form 2 Compound 1 di(hydrogen sulfate) trihydrate. An isolated yield of 80.4% was obtained, with the theoretical yield estimated as 80.3% (based on the concentrations of the extracted mother liquor and wash). The purity of the dried solids was determined as >99.99% area by HPLC and GC indicated about 0.12% by weight residual 1-propanol.

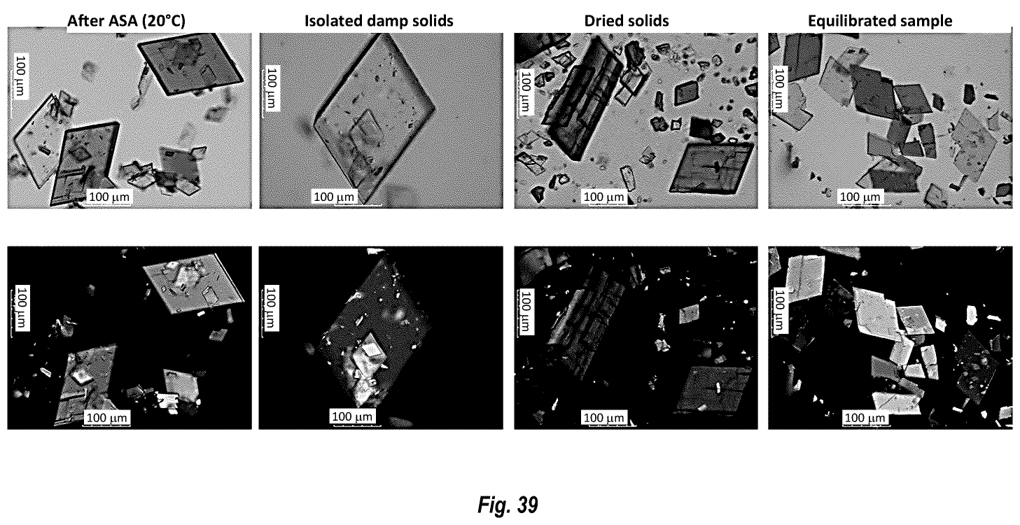

PLM showed the particle to be birefringent after anti-solvent addition at 20° C., as damp solids, after drying, and after moisture equilibration (FIG. 39). By Karl Fischer measurements, a water content on average of 4.33% w/w was measured after about 21 hours of re-exposure to ambient conditions, which increased to 6.07% after another 48 hours. A water content of 6.14% is the theoretical content for a trihydrate.

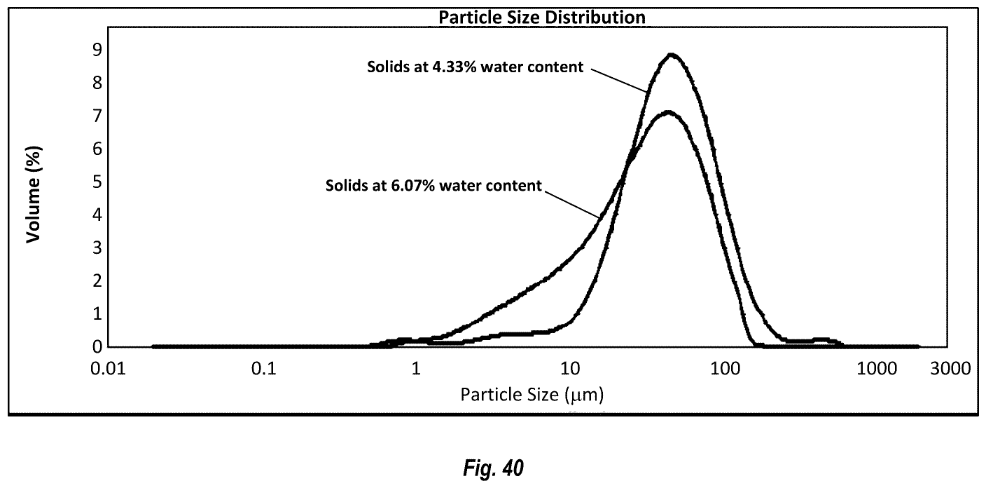

Particle size-measurements were taken before and after complete equilibration (moisture content of 4.33% and then later 6.07%). FIG. 40 is an overlay of the two measurements showing the difference in particle size distributions. The data are summarized in Table 43 below:

TABLE 43
Particle Size Measurements after Equilibration

| | Before complete equilibration; | | After complete equilibration; | |
|---|---|---|---|---|
| | 24 hr post-drying | | 3 days post-drying | |
| | (moisture content = 4.33%) | | (moisture content = 6.07%) | |
| D₁₀ | 17.596 | μm | 6.32 | μm |
| D₅₀ | 46.785 | μm | 32.90 | μm |
| D₉₀ | 109.799 | μm | 84.73 | μm |

These data show that the average particle size decreased with longer equilibration times and higher water contents.

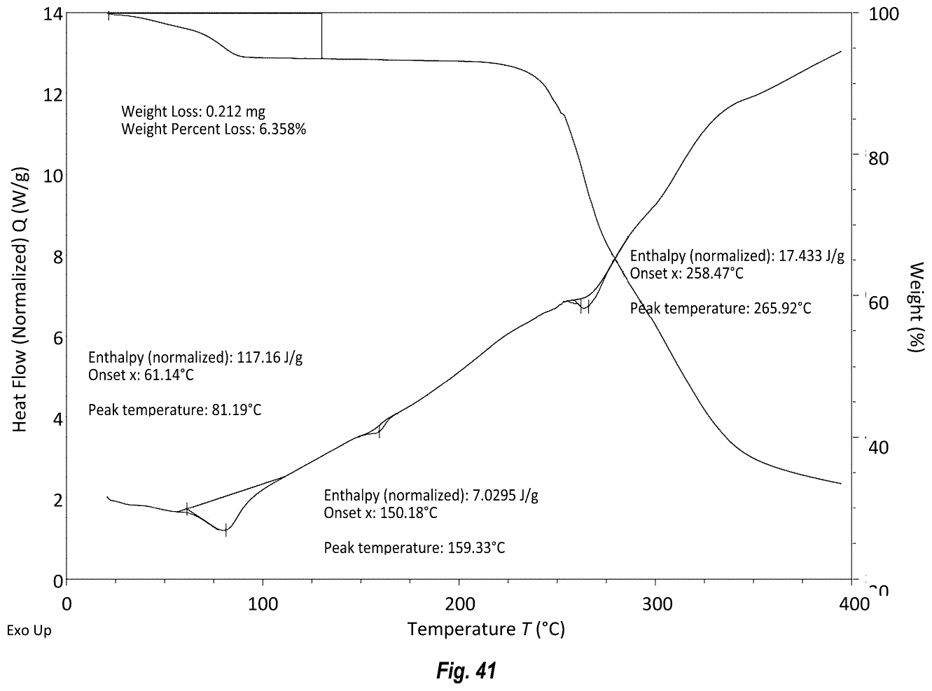

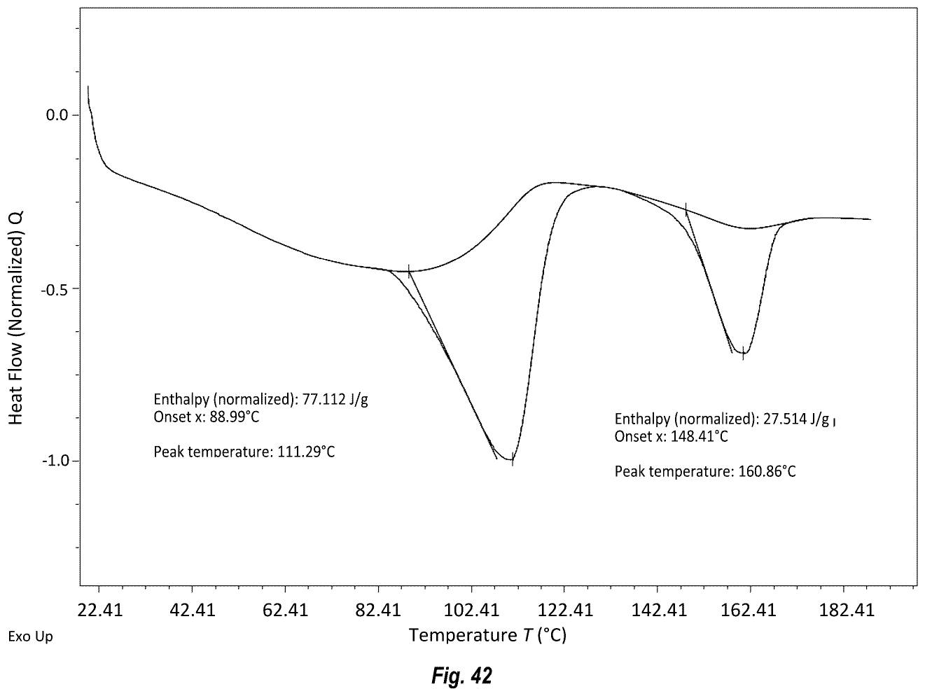

TG/DSC thermograms are presented in FIG. 41 of a fully equilibrated sample of crystalline Form 2 Compound 1 di(hydrogen sulfate) trihydrate. The data reveal a mass loss of about 6.4% between about 20° C. and 130° C., which corresponded with an endothermic event at about 61° C., both of which are likely due to solvent/water loss. A small endothermic event with a peak onset of about 150° C. may be due to melting and the thermal events above 200° C. are likely due to degradation. A DSC thermogram is provided at FIG. 42 showing thermal events at onsets of about 89° C. and about 148° C. The lower one is likely dehydration whereas the higher one is a melting event.

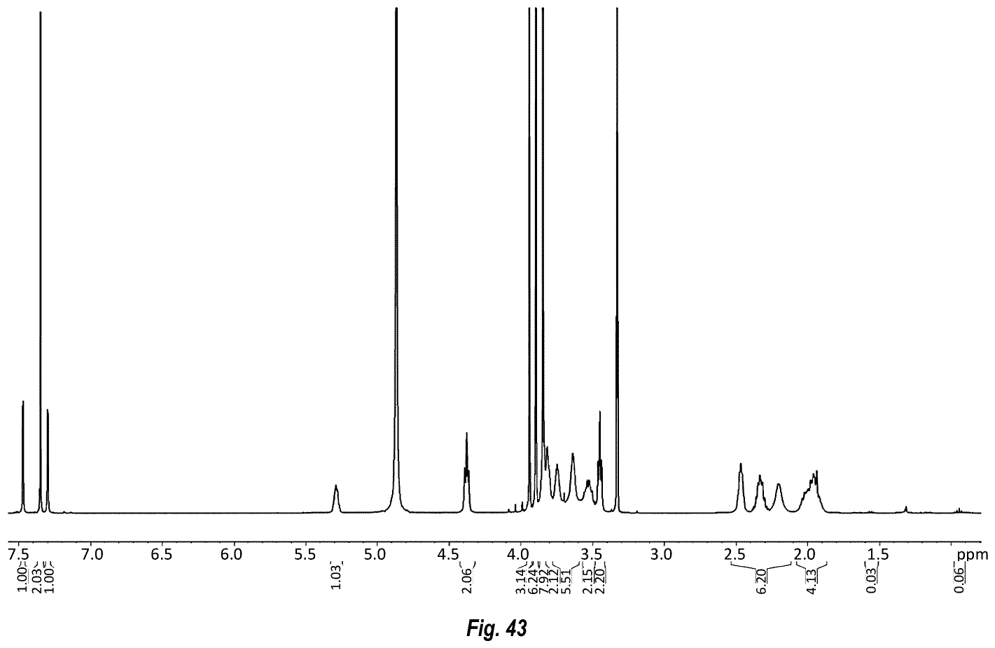

¹H-NMR spectra were collected on crystalline Form 2 Compound 1 di(hydrogen sulfate) trihydrate and the spectrum was consistent with the di(hydrogen sulfate) structure (FIG. 43).

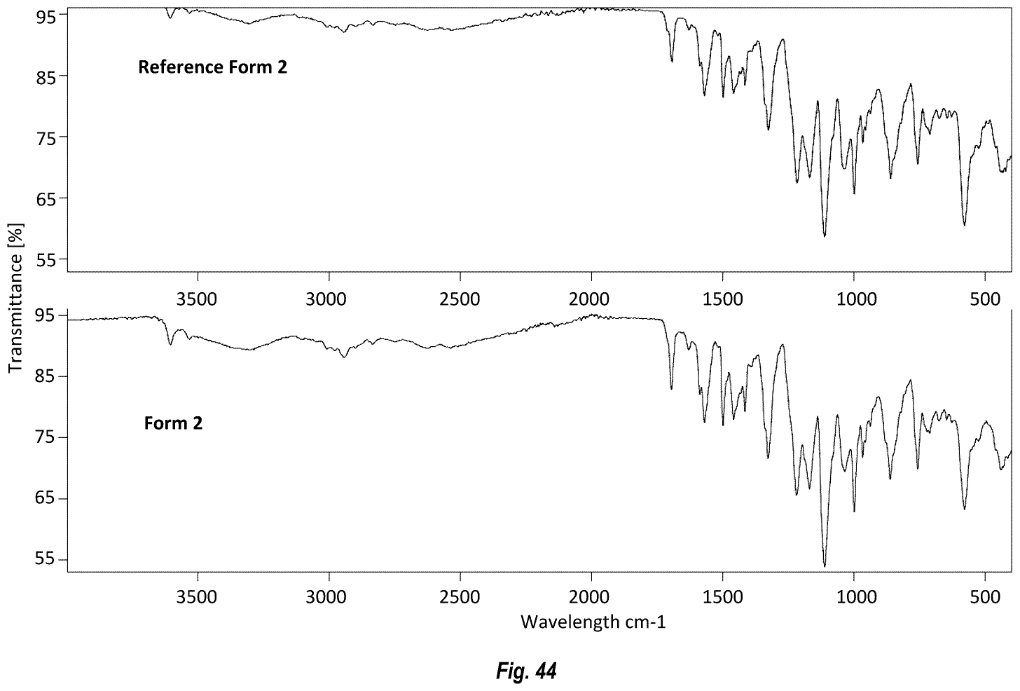

An FT-IR spectrum of crystalline Form 2 Compound 1 di(hydrogen sulfate) trihydrate are shown in FIG. 44.

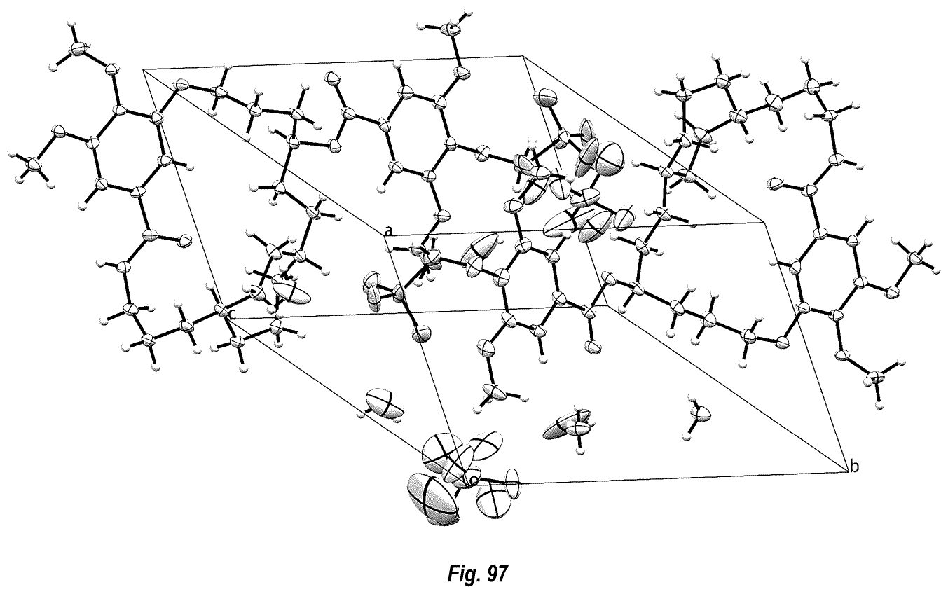

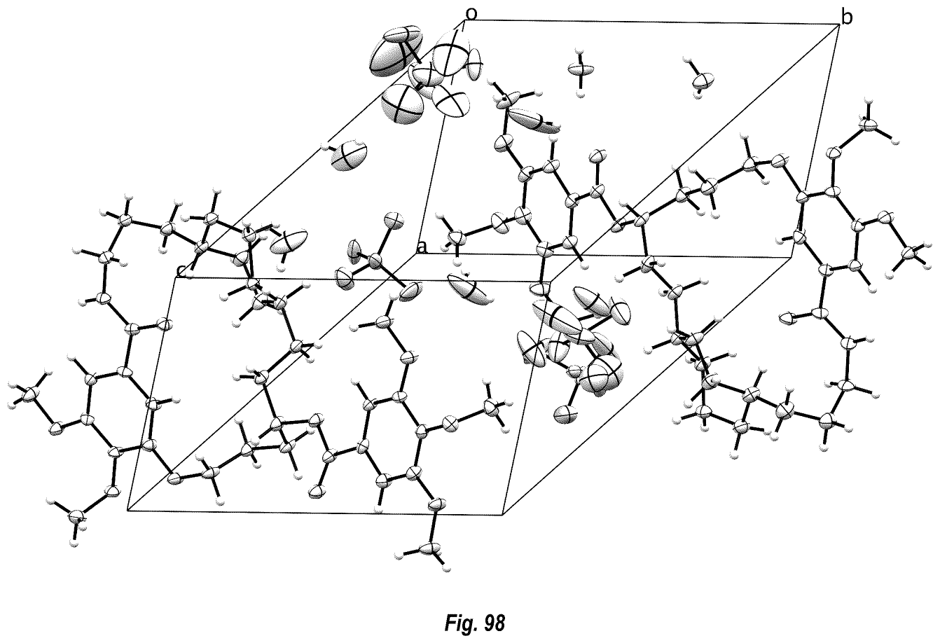

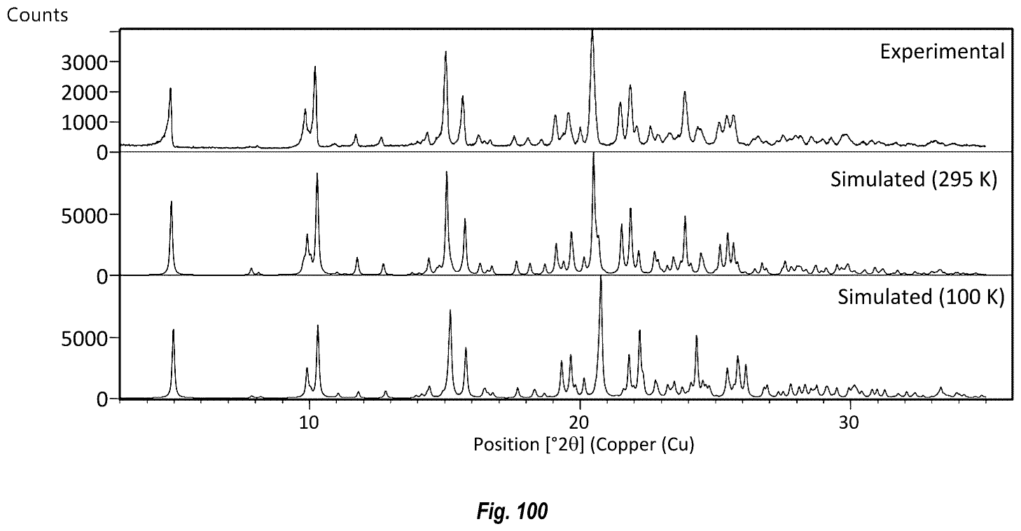

Example 6—Single Crystal Study of Crystalline Form 2 Compound 1 di(hydrogen sulfate) trihydrate: A single crystal of crystalline Form 2 Compound 1 di(hydrogen sulfate) trihydrate was prepared in the following manner. Approximately 10 mg of Amorphous Compound 1 di(hydrogen sulfate) was added to a 2 mL vial, into which 100 μL of 2-ethoxyethanol was added. Complete dissolution was observed at ambient temperature (ca. 20° C.). The solvent was then allowed to evaporate at ambient conditions for 3 days. Post-evaporation, the residual solid was observed to be clear block-like particles, which were analyzed by single crystal X-ray diffraction. FIGS. 97 and 98 show a single crystal structure drawing of crystalline Form 2 Compound 1 di(hydrogen sulfate) trihydrate at 100K in two different orientations. Simulated XRPD diffractograms were calculated for structures collected at 295 K and 100 K, respectively (FIG. 99), and compared to an experimental (room temperature) diffractogram (FIG. 100) with detailed peak list in Table 4 and Table 7 and the 20 most intense peaks listed in Table 5 and Table 8. The simulated diffractogram showed good correlation with the experimental diffractogram indicating that the structure presented in this data is representative of the bulk material.

Example 7-Amorphous Compound 1 di(hydrogen sulfate): Approximately 2.5 g of crystalline Form 2 Compound 1 di(hydrogen sulfate) trihydrate (see, e.g., Example 4) was added to a 250 ml conical flask. 100 mL of methanol was added to the flask to dissolve the solid at ambient temperature. When complete dissolution was observed, the solvent was removed under vacuum via rotary evaporation. An amorphous solid resulted as determined by XRPD (FIG. 45). ¹H-NMR analysis of a solution of the compound prepared from the solid material was consistent with the ¹H-NMR spectrum of Compound 1.

Example 8-Solution Stability of Compound 1 di(hydrogen sulfate): As per Table 44, weighed amounts (mg) of Compound 1 (free base) were added to 5×1.5 mL screw cap vials. Sulfuric acid equivalents were added to each vial as solutions in volumes of 1-propanol: water (80:20% v/v) as further set forth in Table 44. Salt formation occurred in situ. The target free base concentration was about 186 mg/mL. The solutions were stirred at 50° C. for about 24 hours. Clear solutions were observed in all the samples upon set-up. Subsamples (50 μL) of the solutions were extracted for HPLC analysis after 2, 4, 6, and 23 hours of stirring at 50° C. Details are shown in Table 44 below.

TABLE 44
Experimental details for Solubility Stability
of Compound 1 di(hydrogen sulfate)

| | Input | Acid Addition (95%) | | Solvent Addition | |
|---|---|---|---|---|---|
| Solvent | FB | Acid | Vol. | Vol. | |
| System | (mg) | Equiv. | (μL) | (mL) | Observations |
| 1-propanol:water | 119.17 | 1.90 | 20.17 | 0.64 | Clear |
| (80:20% v/v) | 115.93 | 1.95 | 20.14 | 0.62 | Clear |
| | 123.88 | 2.00 | 22.08 | 0.67 | Clear* |
| | 117.46 | 2.05 | 21.45 | 0.63 | Clear* |
| | 120.65 | 2.10 | 22.57 | 0.65 | Clear* |

*nucleation observed after about 23 hours of stirring at 50° C.
FB = Compound 1 (free base)

The results obtained are summarized in Table 45. The concentration of the solutions was determined to be between 162-183 mg/mL by HPLC. Experimental input free base concentration was 186 mg/mL. The solution purity was determined to be between 98.7-99.7% area. The purity of the samples analyzed at the T₂₃ₕ timepoint were observed to be slightly lower than the earlier samples taken (by about 0.8% area). Overall, the results indicated substantial stability for Compound 1 di(hydrogen sulfate) in the 1-propanol:water solvent system at 50° C.

TABLE 45
Results obtained from solution stability studies using
1-propanol:water (80:20% v/v) solvent system

| | Acid | HPLC Conc. (mg/mL) | | | | HPLC Purity (% area) | | | |
|---|---|---|---|---|---|---|---|---|---|
| Solvent System | Equiv. | T_{2 h} | T_{4 h} | T_{6 h} | T_{23 h} | T_{2 h} | T_{4 h} | T_{6 h} | T_{23 h} |
| 1-propanol:water | 1.90 | 176 | 167 | 183 | 164 | 99.70 | 99.67 | 99.58 | 98.94 |
| (80:20% v/v) | 1.95 | 175 | 169 | 179 | 162 | 99.60 | 99.61 | 99.53 | 99.00 |
| | 2.00 | 170 | 165 | 169 | 170 | 99.66 | 99.58 | 99.43 | 98.78 |
| | 2.05 | 166 | 165 | 173 | 165 | 99.68 | 99.52 | 99.42 | 98.85 |
| | 2.10 | 166 | 164 | 168 | 167 | 99.60 | 99.62 | 99.47 | 98.89 |

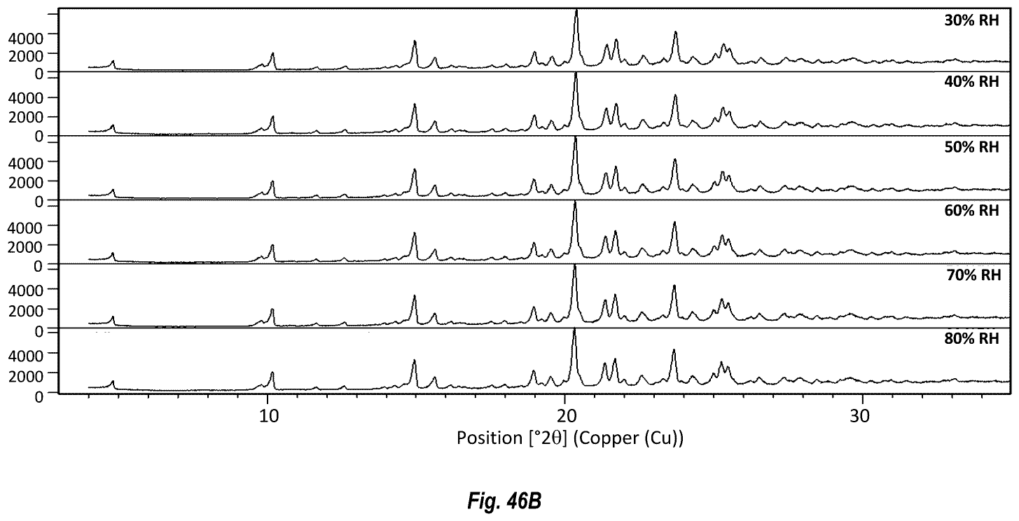

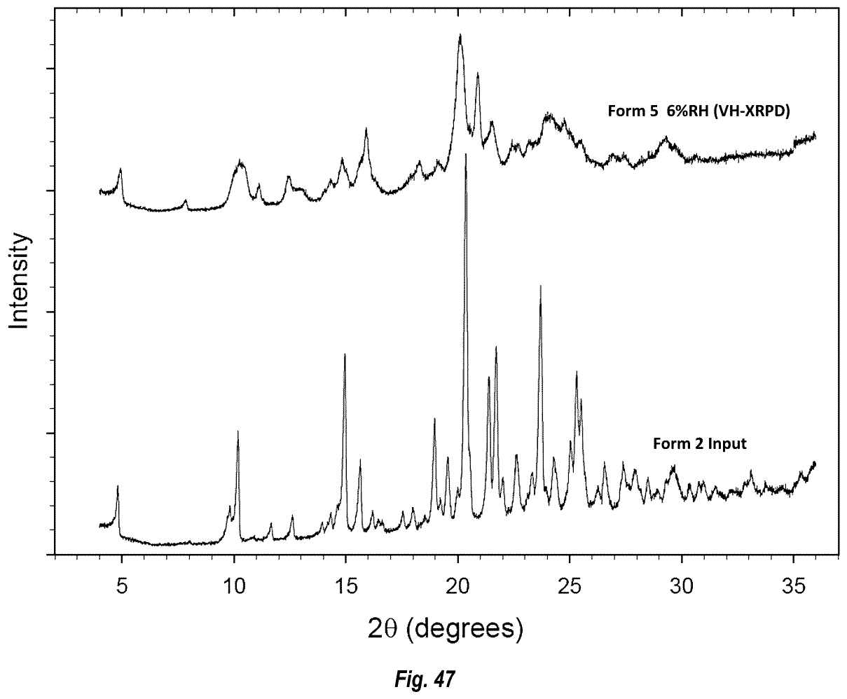

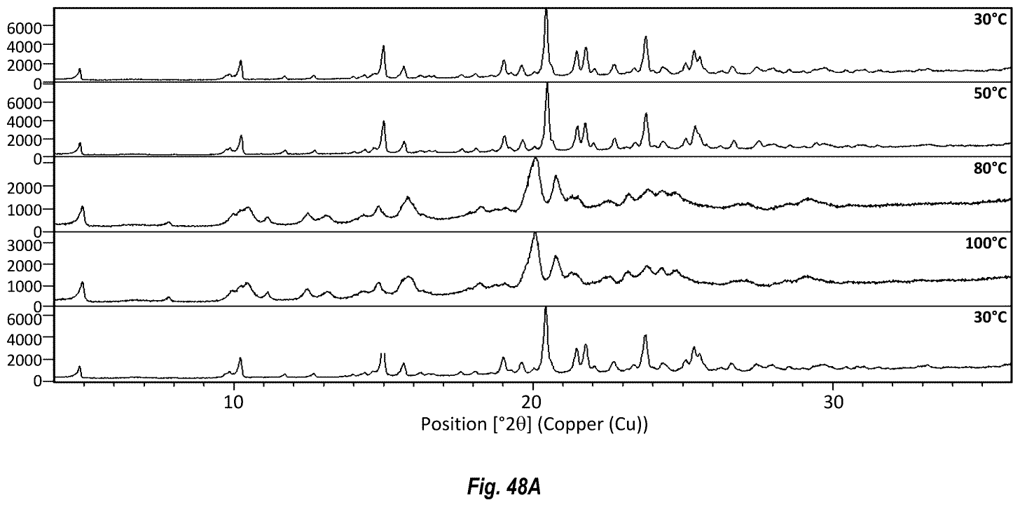

Example 9—Variable Temperature and Humidity Stability of Crystalline Form 2 Compound 1 di(hydrogen sulfate) trihydrate: A variable-humidity analysis was conducted on crystalline Form 2 Compound 1 di(hydrogen sulfate) trihydrate prepared in accordance with Example 3. Between 10% and 80% RH, crystalline Form 2 Compound 1 di(hydrogen sulfate) trihydrate was stable and no form change was observed by XRPD (FIGS. 46A and 46B). At 6% RH, however, XRPD indicated a different form (referred to as crystalline Form 5 Compound 1 di(hydrogen sulfate)) (FIG. 47). Upon exposure to relative humidities of 10% or greater, crystalline Form 5 Compound 1 di(hydrogen sulfate) converted to crystalline Form 2 Compound 1 di(hydrogen sulfate) trihydrate. A table summarizing the results (Table 46) is set forth below.

TABLE 46
Results of VH-XRPD analysis for Crystalline Form
2 Compound 1 di(hydrogen sulfate) trihydrate

| | Rel. Humidity | Hold time | |
|---|---|---|---|
| Cycle | (% RH) | (min) | XRPD Results |
| Desorption | 40 | 60 | 2 |
| | 20 | 60 | 2 |
| | 10 | 60 | 2 |
| | 6 | 60 | 5, PC |
| Sorption | 10 | 60 | 2 |
| | 20 | 60 | 2 |
| | 30 | 60 | 2 |
| | 40 | 960 | 2 |
| | 50 | 60 | 2 |
| | 60 | 60 | 2 |
| | 70 | 60 | 2 |
| | 80 | 60 | 2 |

2 Crystalline Form 2 di(hydrogen sulfate) trihydrate
5 Crystalline Form 5 di(hydrogen sulfate)
PC Poorly Crystalline

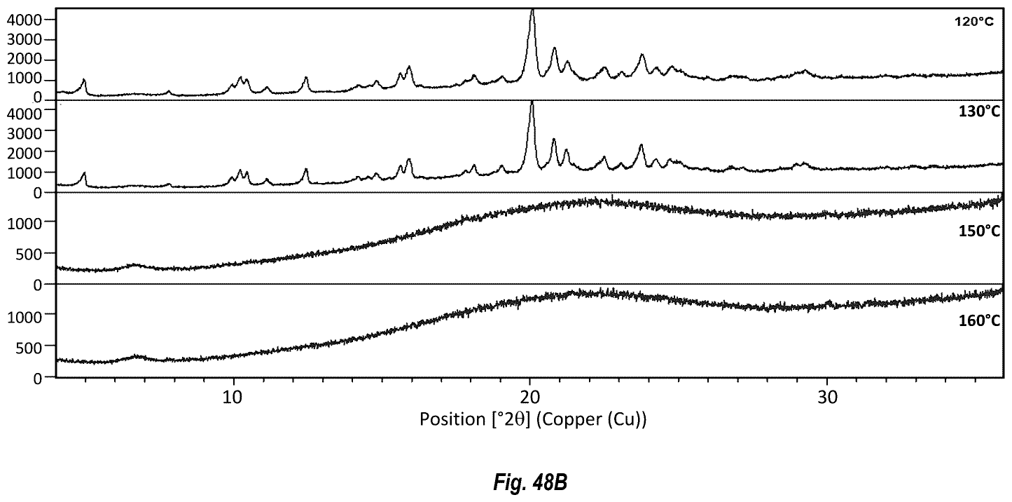

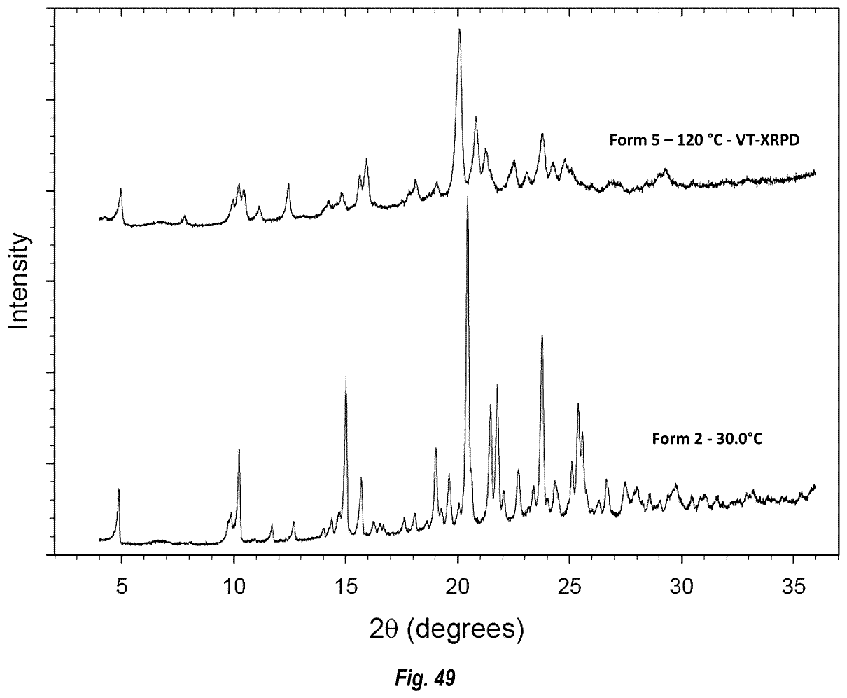

A variable-temperature study was also conducted on crystalline Form 2 Compound 1 di(hydrogen sulfate) trihydrate prepared in accordance with Example 3. A sample of crystalline Form 2 Compound 1 di(hydrogen sulfate) trihydrate was heated between 30° C. and 160° C. Crystalline Form 2 Compound 1 di(hydrogen sulfate) trihydrate was retained between 30° C. and 50° C. as determined by XRPD (FIGS. 48A and 48B). At temperatures 80° C. to 130° C., the sample converted to crystalline Form 5 Compound 1 di(hydrogen sulfate) (FIG. 49). The sample melted at 150° C. become amorphous. The data are summarized in Table 47 below.

TABLE 47
Results of VT-XRPD analysis for Crystalline Form
2 Compound 1 di(hydrogen sulfate) trihydrate

| Cycle | Temp (° C.) | Hold Time (min) | XRPD Results |
|---|---|---|---|
| Heating | 30 | 0 | 2 |
| | 50 | 30 | 2 |
| | 80 | 30 | 5+ |
| | 100 | 30 | 5+ |
| Cooling | 30 | 10 | 2 |
| Heating | 120 | 30 | 5 |
| | 130 | 30 | 5 |
| | 150 | 30 | Melt |
| | 160 | 30 | Melt |

2 Crystalline Form 2 di(hydrogen sulfate) trihydrate
5 Crystalline Form 5 di(hydrogen sulfate)
+ Extra peaks
Melt Melt/Amorphous

Example 10—Amorphous Compound 1 (free base); Amorphous Compound 1 (free base) was used to prepare different crystalline forms. Compound 1 (free base) can be prepared as shown in, for example, WO 2021/204896 (“Method A”—see also Scheme 1 above). Compound 1 (free base) may also be prepared as described by any of the methods below (i.e., Methods B-E—see also Schemes 2-4 above). See also WO 2023/056910.

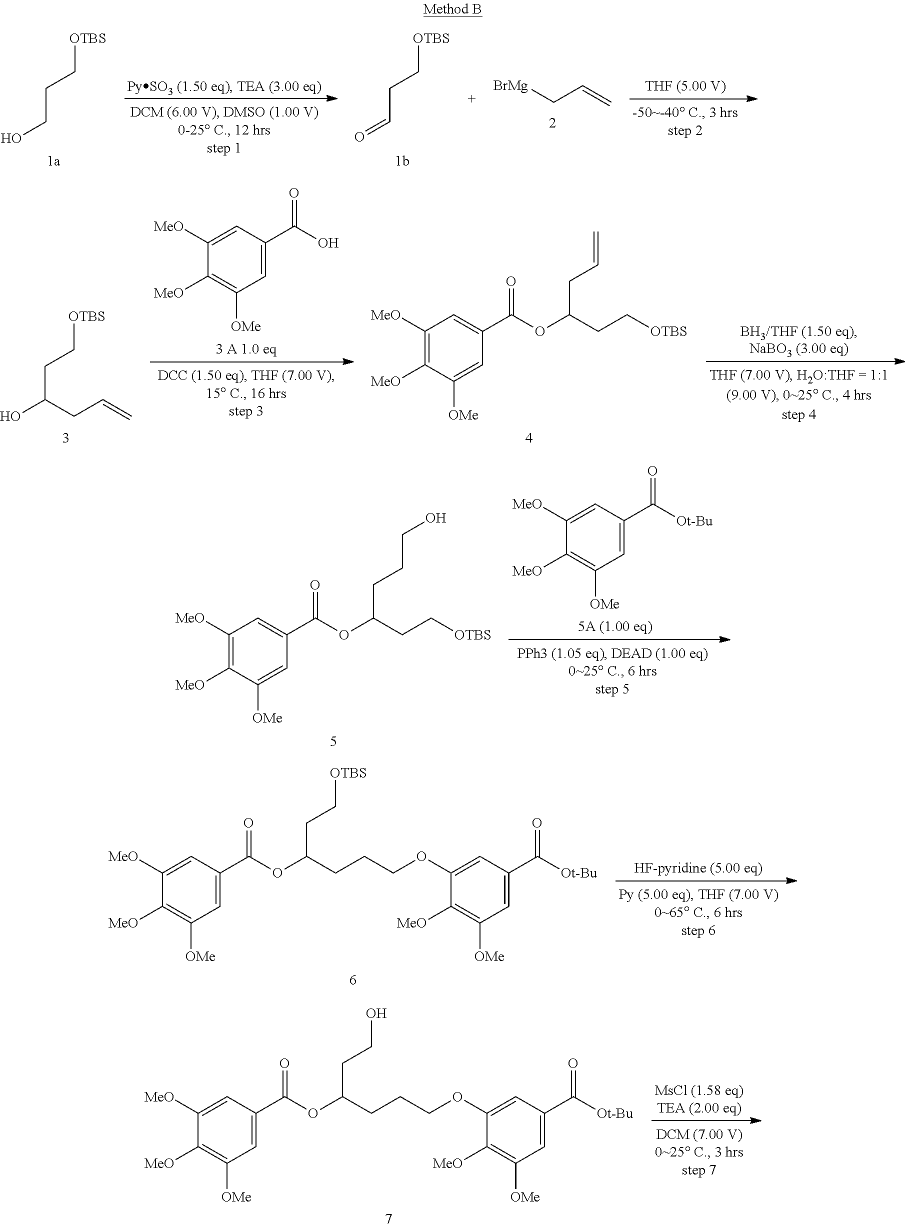

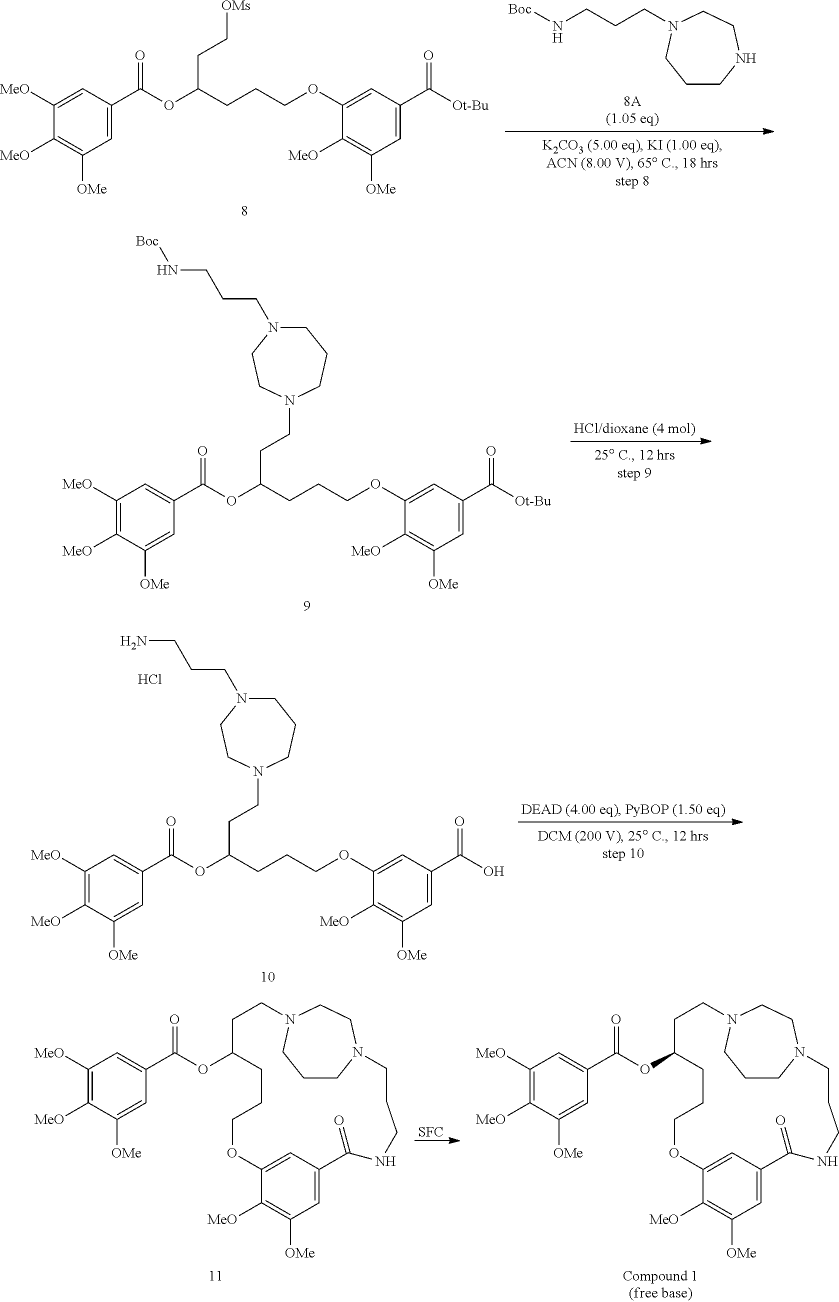

### Step 1:

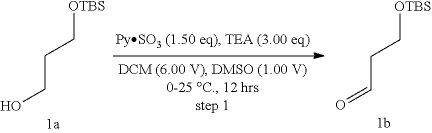 [SMILES: CC(C)(C)[Si](C)(C)OCCCO ; CC(C)(C)[Si](C)(C)OCCC=O]

At 0-5° C., Compound 1a (3.50 kg, 18.41 mol, 1.00 eq), DCM (21.0 L) and DMSO (3.50 L) were charged into the reactor. Then TEA (5.58 kg, 55.1 mol, 3.00 eq), Py.SO3 (4.39 kg, 27.5 mol, 1.50 eq) into the mixture at 0-20° C. The reaction mixture was stirred at 20-25° C. for 12 hrs. An aqueous solution of 0.5 M citric acid (20.0 L) was slowly added into the mixture at 0˜20° C. and stirred for 10 min. The organic phase was separated, washed with an aqueous solution of 10% NaHCO₃ (20.0 L) and brine (20.0 L), dried over Na₂SO₄, filtered and concentrated to give the compound 1b (3.60 kg, crude) as brown oil. Purity determined by quantitative NMR: 66.8%

¹H NMR (400 MHz, CDCl₃) δ 9.79 (d, J=2.0 Hz, 1H), 3.97 (t, J=2.0 Hz, 2H), 2.57 (t, J=6.0 Hz, 2H), 0.89 (s, 9H), 0.05 (s, 6H).

### Step 2:

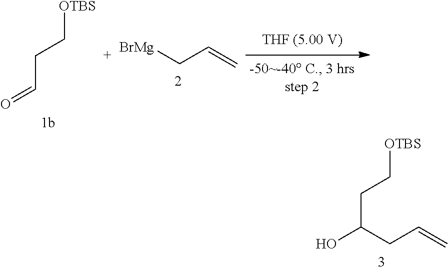

At 20° C., THF (28.8 L), Compound 2 (1 M, 22.9 L, 1.20 eq) was charged into the reactor then cooled −60˜−50° C. Compound 1b (3.60 kg, 19.12 mol, 1.00 eq) in THF (7.20 L) was added into the mixture at −60˜−50° C. The reaction mixture was stirred at −50˜−40° C. for 3 hrs then slowly warmed to 0-10° C. The reaction was quenched by addition of an aqueous solution of 0.5 N HCl (20.0 L) between 0-10° C. The organic phase was separated, washed with brine (20.0 L), dried over Na₂SO₄, filtered and concentrated to give a brown oil. The oil was purified by column chromatography (SiO₂, n-hexane/ethyl acetate=1/0 to 50/1) to give the compound 3 (2.10 kg, 9.11 mol, 48.0% yield) as yellow oil. Note: Changed the charging sequence by adding the aldehyde to Grignard reagent, the yield increased from 40% to 48%.

¹H NMR (400 MHz, CDCl₃) δ 5.72-5.94 (m, 1H), 4.97-5.18 (m, 2H), 3.75-3.94 (m, 3H), 3.37 (d, J=6.4 Hz, 1H), 2.17-2.31 (m, 2H), 1.60-1.71 (m, 2H), 0.88 (s, 9H), 0.03 (s, 6H).

### Step 3:

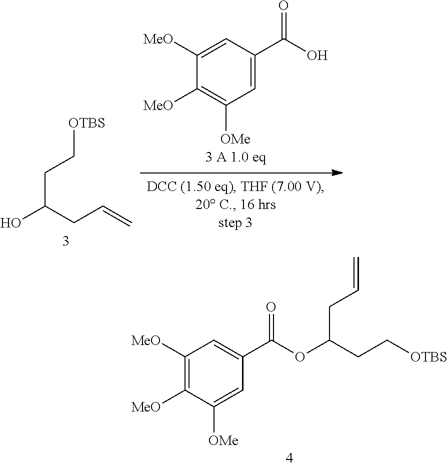 [SMILES: C=CCC(O)CCO[Si](C)(C)C(C)(C)C ; COc1cc(C(=O)O)cc(OC)c1OC ; C=CCC(CCO[Si](C)(C)C(C)(C)C)OC(=O)c1cc(OC)c(OC)c(OC)c1]

At 20-25° C., THF (14.70 L), Compound 3 (2.10 kg, 9.11 mol, 1.00 eq), Compound 3 A (1.93 kg, 9.11 mol, 1.00 eq), DCC (2.82 kg, 13.69 mol, 487 mL, 1.50 eq), DMAP (1.67 kg, 13.69 mol, 1.50 eq) were charged into the reactor. The reaction mixture was stirred at 20˜25° C. for 16 hrs. The reaction mixture was filtered and the filtrate concentrated under reduced pressure to give the residue. The residue was purified by column chromatography (n-hexane/ethyl acetate=100/0 to 90/10) to give the compound 4 (2.70 kg, 6.36 mol, 70% yield, 95.1% purity) as yellow oil.

¹H NMR (400 MHz, CDCl₃) δ 7.28 (s, 2H), 5.74-5.92 (m, 1H), 5.22-5.33 (m, 1H), 5.02-5.17 (m, 2H), 3.90 (s, 9H), 3.67-3.76 (m, 2H), 2.41-2.57 (m, 2H), 1.87-2.00 (m, 2H), 0.89 (s, 9H), 0.05 (s, 6H).

### Step 4:

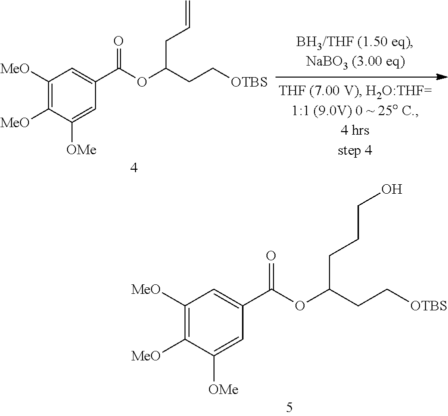 [SMILES: C=CCC(CCO[Si](C)(C)C(C)(C)C)OC(=O)c1cc(OC)c(OC)c(OC)c1 ; COc1cc(C(=O)OC(CCCO)CCO[Si](C)(C)C(C)(C)C)cc(OC)c1OC]

At 10-20° C., Compound 4 (2.40 kg, 5.66 mol, 1.00 eq) and THF (16.80 L) was charged into a 50.0 L reactor. BH3. THF (1 M, 8.48 L, 1.50 eq) was added dropwise into the mixture at 0˜10° C. A mixture of H₂O (10.8 L) and THF (10.8 L) was added to quench the reaction between 0˜10° C. (Caution: evolution of H₂, and exothermal is observed.). NaBO₃·4H₂O (2.61 kg, 16.9 mol, 3.00 eq) was charged by portions into the mixture at 0˜10° C., then the reaction mixture was stirred at 10˜25° C. for 4 hrs. The reaction was quenched by addition of an aqueous solution of 10% Na₂S₂O₃ (20.0 L) slowly at 0˜10° C. Ethyl acetate (7.50 L) was charged into the reactor at 10˜20° C. and stirred for 10 min. The organic phase was separated, washed with brine (5.00 L), dried over Na₂SO₄, filtered and concentrated to give the residue. The residue was purified by column chromatography (SiO₂, petroleum ether: ethyl acetate=10:1 to 1:1) to give the compound 5 (1.40 kg, 3.38 mol, 60% yield, 91.6% purity) as yellow oil.

¹H NMR (400 MHz, CDCl₃) δ 7.26 (s, 2H), 5.16-5.33 (m, 1H), 3.88 (s, 9H), 3.55-3.78 (m, 4H), 1.56-2.01 (m, 6H), 0.87 (s, 9H), 0.02 (s, 6H).

### Step 5:

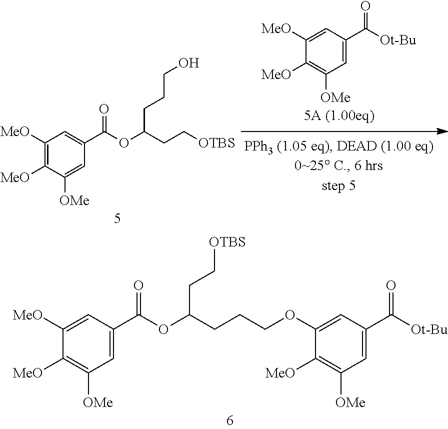 [SMILES: COc1cc(C(=O)OC(CCCO)CCO[Si](C)(C)C(C)(C)C)cc(OC)c1OC ; COc1cc(C(=O)OC(C)(C)C)cc(OC)c1OC ; COc1cc(C(=O)OC(CCCOc2cc(C(=O)OC(C)(C)C)cc(OC)c2OC)CCO[Si](C)(C)C(C)(C)C)cc(OC)c1OC]

At 20° C., Compound 5 (1.48 kg, 3.34 mol, 1.00 eq) and Toluene (10.3 L), compound 5A (0.85 kg, 3.34 mol, 1.00 eq), PPh3 (0.91 kg, 3.51 mol, 1.05 eq) were charged into the reactor. DEAD (0.58 kg, 3.34 mol, 1.00 eq) was added dropwise, (Exothermic phenomenon is observed during the addition process). After addition, the reaction mixture was stirred at 25° C. for 6 hrs, then the reaction mixture was stirred at −20° C. for 1 hr to precipitate part of OPPh3. The reaction mixture was filtered and the filtrate concentrated under reduced pressure to give crude product. The crude product was purified by silica gel chromatography (n-hexane/Ethyl acetate=5/1) to give the compound 6 (1.38 kg, 2.03 mol, 81.2% purity) as colorless oil.

¹H NMR (400 MHz, CDCl₃) δ 7.29 (s, 2H), 7.22 (d, J=1.6 Hz, 2H) 5.27-5.37 (m, 1H), 4.06 (s, 2H), 3.84-3.95 (m, 15H), 3.67-3.78 (m, 2H), 1.87-2.06 (m, 6H), 1.65 (s, 9H), 0.89 (s, 9H), 0.02 (s, 6H).

### Step 6:

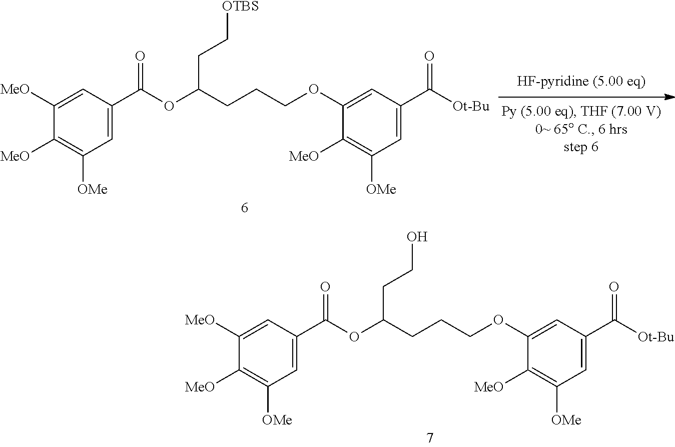 [SMILES: COc1cc(C(=O)OC(CCCOc2cc(C(=O)OC(C)(C)C)cc(OC)c2OC)CCO[Si](C)(C)C(C)(C)C)cc(OC)c1OC ; COc1cc(C(=O)OC(CCO)CCCOc2cc(C(=O)OC(C)(C)C)cc(OC)c2OC)cc(OC)c1OC]

At 20° C., Compound 6 (1.35 kg, 1.98 mol, 1.00 eq) and THF (9.45 L) were charged into the reactor. Pyridine (0.78 kg, 9.95 mol, 5.00 eq), HF-Pyridine (1.40 kg, 9.95 mol, 70% purity, 5.00 eq) were added to the reaction mixture at 0-10° C. The reaction mixture was stirred at 60˜65° C. for 6 hrs. An aqueous solution of 1 M citric acid (˜16.00 L) was added to the reaction mixture at 0˜20° C. and stirred for 10 min. The organic layer's pH was adjusted to pH ˜8 by addition of an aqueous solution of 10% NaHCO₃ (˜16.00 L). The organic layer was washed with brine (16.0 L), dried over Na₂SO₄, filtered and concentrated to give the compound 7 (1.09 kg, 81.0% purity) as yellow oil.

¹H NMR (400 MHz, CDCl₃) δ 7.30 (s, 2H), 7.21-7.25 (m, 2H), 5.33-5.45 (m, 1H), 4.05-4.12 (m, 2H), 3.86-3.94 (m, 15H), 3.58-3.77 (m, 2H), 1.88-2.04 (m, 6H), 1.58 (s, 9H).

### Step 7:

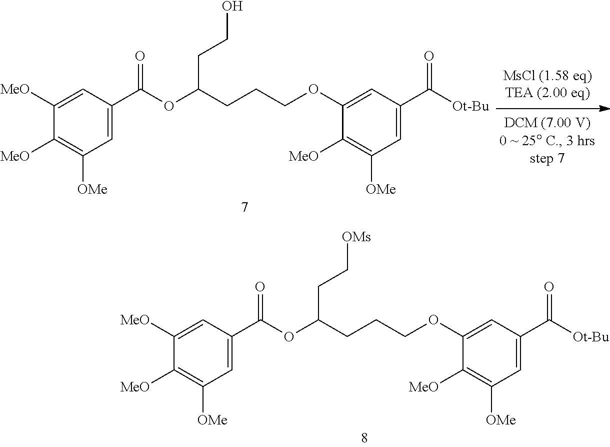 [SMILES: COc1cc(C(=O)OC(CCO)CCCOc2cc(C(=O)OC(C)(C)C)cc(OC)c2OC)cc(OC)c1OC ; COc1cc(C(=O)OC(CCCOc2cc(C(=O)OC(C)(C)C)cc(OC)c2OC)CCOS(C)(=O)=O)cc(OC)c1OC]

At 0° C., Compound 7 (1.03 kg, 1.82 mol, 1.00 eq) and DCM (7.21 L) was charged into a 20.0 L reactor, then TEA (0.37 kg, 3.64 mol, 2.00 eq) was added. MsCl (0.33 kg, 2.88 mol, 1.58 eq) was added dropwise the reaction mixture at 0-5° C. The reaction mixture was stirred at 15˜25° C. for 3 hrs. An aqueous solution of 1 M citric acid (6.00 L) was slowly added to quench the reaction at 0˜20° C. and stirred for 10 min. The aqueous phase was separated. The organic layer was adjusted to pH=8 with an aqueous solution of 10% NaHCO₃(6.00 L). The organic phase was separated, washed with brine (6.00 L), dried over Na₂SO₄, filtered and concentrated to give the compound 8 (1.14 kg, 1.77 mol, crude, 82% purity) as brown oil. Purity determined by quantitative NMR: 87.3%

¹H NMR (400 MHz, CDCl₃) δ 7.28 (s, 2H), 7.19-7.25 (m, 2H), 5.3-5.43 (m, 1H), 4.26-4.40 (m, 2H), 4.04-4.12 (m, 2H), 3.84-3.93 (m, 15H), 2.98 (s, 3H), 2.21 (q, J=6.0 Hz, 2H), 1.87-2.00 (m, 4H), 1.58 (s, 9H).

### Step 8:

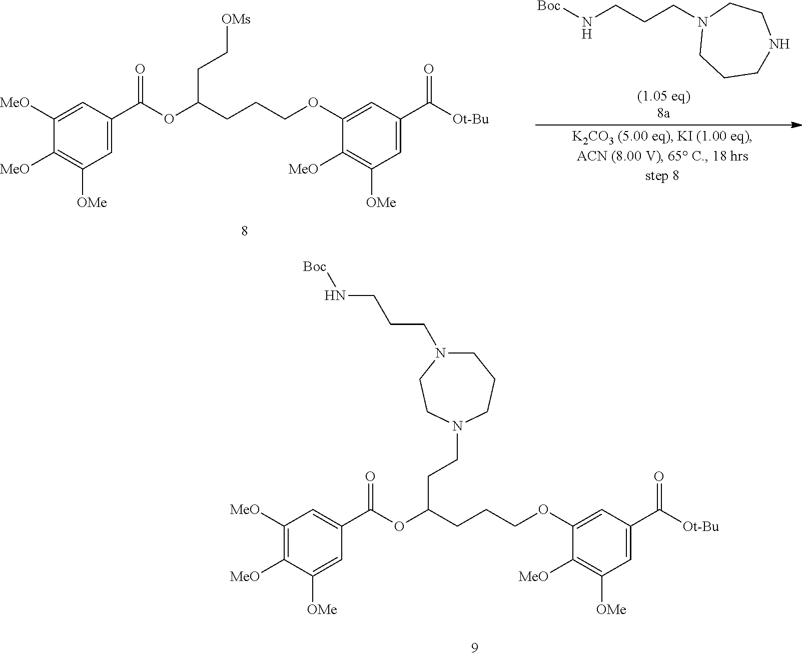 [SMILES: COc1cc(C(=O)OC(CCCOc2cc(C(=O)OC(C)(C)C)cc(OC)c2OC)CCOS(C)(=O)=O)cc(OC)c1OC ; CC(C)(C)OC(=O)NCCCN1CCCNCC1 ; COc1cc(C(=O)OC(CCCOc2cc(C(=O)OC(C)(C)C)cc(OC)c2OC)CCN2CCCN(CCCNC(=O)OC(C)(C)C)CC2)cc(OC)c1OC]

At 25° C., Compound 8 (1190 g, 1.85 mol, 1.00 eq) and ACN (9.52 L) were charged into a 20.0 L reactor. Then, compound 8A (548 g, 2.13 mol, 1.05 eq), K₂CO₃ (1279 g, 9.26 mol, 5.00 eq) and KI (307 g, 1.85 mol, 1.00 eq) were added. The reaction mixture was stirred at 65° C. for 18 hrs. The solvent was removed under reduced pressure to give the residue. H₂O (3.00 L) was added to the residue and extracted with EtOAc (3.00 L×3). The organic phase was separated, washed with brine (3.00 L), dried over Na₂SO₄, filtered and concentrated to give a residue. The residue was purified by column chromatography (SiO₂, Dichloromethane: Methanol=50/1 to 5/1) to give the compound 9 (1116 g, 1.39 mol, 75.0% yield) as yellow oil. Purity determined by quantitative NMR: 91.8%

¹H NMR (400 MHz, CDCl₃) δ 7.30 (s, 2H), 7.21 (s, 2H), 5.23-5.36 (m, 1H), 4.04-4.17 (m, 2H), 3.73-3.94 (m, 15H), 3.06 (t, J=6.8 Hz, 2H), 2.65-2.80 (m, 8H), 2.60 (t, J=7.6 Hz, 2H), 2.49 (t, J=7.6 Hz, 2H), 1.86-2.03 (m, 6H), 1.76-1.85 (m, 2H), 1.61-1.68 (m, 2H), 1.58 (s, 9H), 1.43 (s, 9H).

### Step 9:

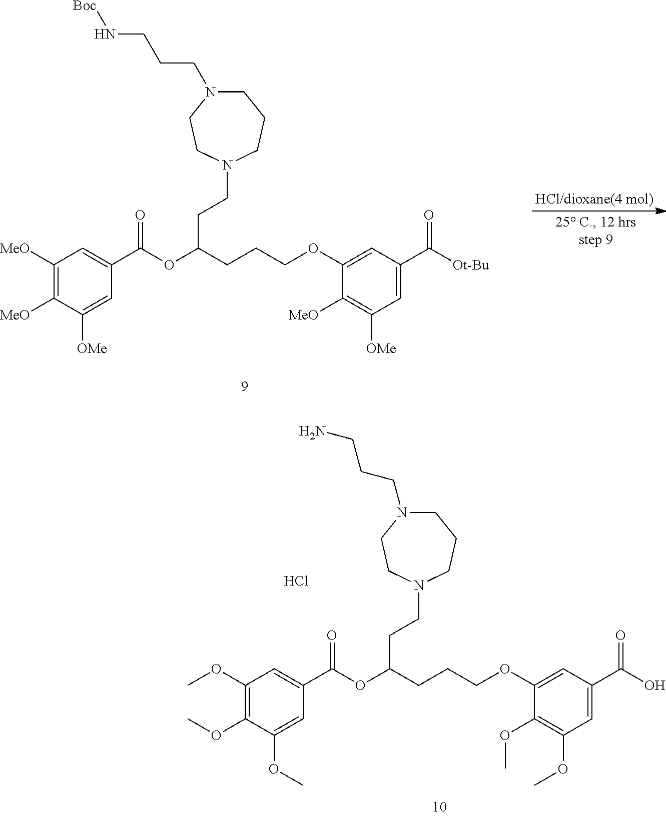 [SMILES: COc1cc(C(=O)OC(CCCOc2cc(C(=O)OC(C)(C)C)cc(OC)c2OC)CCN2CCCN(CCCNC(=O)OC(C)(C)C)CC2)cc(OC)c1OC ; COc1cc(C(=O)OC(CCCOc2cc(C(=O)O)cc(OC)c2OC)CCN2CCCN(CCCN)CC2)cc(OC)c1OC ; Cl]

At 0-5° C., a solution of HCl in dioxane (4 mol, 7.60 L) and Compound 9 (1086 g, 1.35 mol, 1.00 eq) was charged into a 20.0 L reactor. The reaction mixture was stirred at 25° C. for 12 hrs. The solvent was removed under reduced pressure to give the compound 10 (1050 g, as HCl Salt) as yellow solid. Purity determined by quantitative NMR: 75.2%

¹H NMR (400 MHz, CD₃OD) δ 7.29 (s, 2H), 7.26 (s, 2H) 5.2-5.37 (m, 1H), 4.11 (s, 2H), 3.94 (brs, 4H), 3.78-3.90 (m, 15H), 3.33-3.45 (m, 4H), 3.08 (t, J=7.6 Hz, 2H), 2.12-2.49 (m, 6H), 1.90-2.09 (m, 4H).

### Step 10:

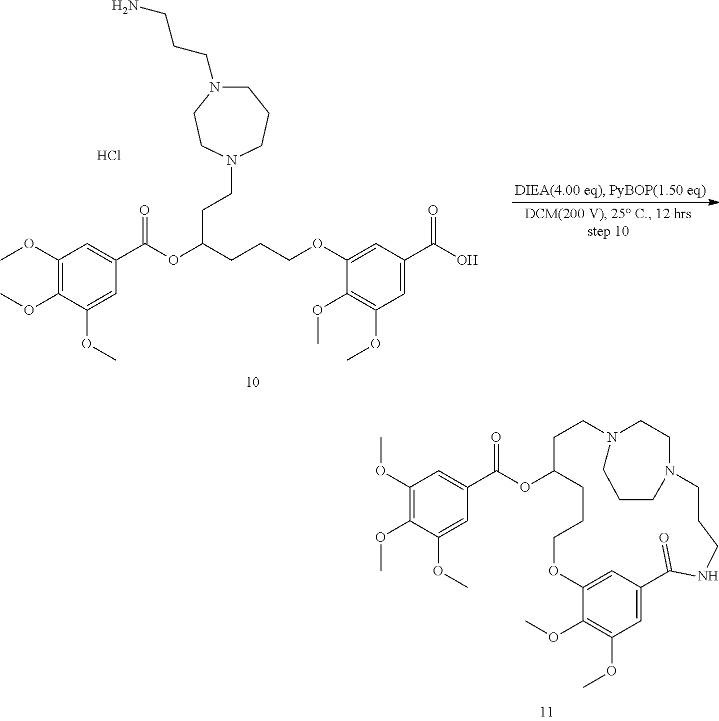 [SMILES: COc1cc(C(=O)OC(CCCOc2cc(C(=O)O)cc(OC)c2OC)CCN2CCCN(CCCN)CC2)cc(OC)c1OC ; Cl ; COc1cc(C(=O)OC2CCCOc3cc(cc(OC)c3OC)C(=O)NCCCN3CCCN(CC2)CC3)cc(OC)c1OC]

At 20° C., Compound 10 (1050 g, 1.53 mol, 1.00 eq, HCl) and DCM (210 L) were charged into the reactor. Then, DIEA (793 g, 6.13 mol, 4.00 eq) and PyBOP (38.4 g, 2.29 mol, 1.50 eq) was added to the reactor at 20° C. The reaction mixture was stirred at 25° C. for 12 hrs. The reaction mixture was concentrated at 35-40° C. to give the residue. The residue was triturated with MeOH (4.2 L, 4.00 V) at 20° C. for 60 min. The mixture was filtered and the cake collected to give the compound 11 (470 g, 34.94 mmol, 48.6% yield) as white solid. Purity determined by quantitative NMR: 75.2%

¹H NMR (400 MHz, CD₃OD) δ 7.31 (s, 2H), 7.20 (d, J=1.8 Hz, 1H), 7.13 (d, J=1.8 Hz, 1H), 5.49 (s, 1H), 4.31 (br d, J=8.3 Hz, 1H), 4.18 (br s, 1H), 3.85-3.89 (m, 9H), 3.81 (d, J=7.3 Hz, 6H), 3.56-3.66 (m, 1H), 3.38-3.49 (m, 1H), 2.97 (td, J=3.2, 10.3 Hz, 1H), 2.84-2.91 (m, 2H), 2.74-2.84 (m, 3H), 2.61-2.73 (m, 4H), 2.56 (br t, J=6.5 Hz, 2H), 1.86-1.95 (m, 5H), 1.73-1.85 (m, 5H).

SFC-Chiral Separation of Compound 1:

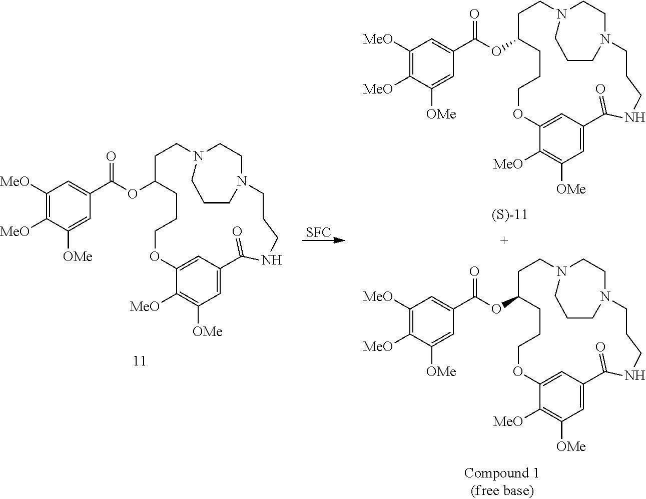

Enantiomers of the racemic compound 11 (470 g) were separated by chiral-SFC (Supercritical Fluid Chromatography-Column: Phenomenex-Cellulose-2 (250 mm*30 mm, 10 um); mobile phase: [0.1% NH₃—H₂O MEOH]; B %: 60%-60%, 10 min) to give the compound (S)-11 (170 g) and Compound 1 (165 g) as white solids.

### Compound (S)-11:

LCMS (ESI position ion) m/z: 630.2 (M+H)+ (calculated: 630.3), purity >99%
Chiral HPLC: retention time=3.836 min, ee >99%

¹H NMR (400 MHz, CD₃OD) δ 7.31 (s, 2H), 7.20 (d, J=1.8 Hz, 1H), 7.13 (d, J=1.8 Hz, 1H), 5.49 (s, 1H), 4.31 (br d, J=8.3 Hz, 1H), 4.18 (br s, 1H), 3.85-3.89 (m, 9H), 3.81 (d, J=7.3 Hz, 6H), 3.56-3.66 (m, 1H), 3.38-3.49 (m, 1H), 2.97 (td, J=3.2, 10.3 Hz, 1H), 2.84-2.91 (m, 2H), 2.74-2.84 (m, 3H), 2.61-2.73 (m, 4H), 2.56 (br t, J=6.5 Hz, 2H), 1.86-1.95 (m, 5H), 1.73-1.85 (m, 5H).

Compound 1:
LCMS (ESI position ion) m/z: 630.2 (M+H)+ (calculated: 630.3), purity >99%
Chiral SFC: retention time=6.560 min, ee >99%

¹H NMR (400 MHz, CD₃OD) δ 7.31 (s, 2H), 7.20 (d, J=1.8 Hz, 1H), 7.13 (d, J=1.8 Hz, 1H), 5.49 (s, 1H), 4.31 (br d, J=8.3 Hz, 1H), 4.18 (br s, 1H), 3.85-3.89 (m, 9H), 3.81 (d, J=7.3 Hz, 6H), 3.56-3.66 (m, 1H), 3.38-3.49 (m, 1H), 2.97 (td, J=3.2, 10.3 Hz, 1H), 2.84-2.91 (m, 2H), 2.74-2.84 (m, 3H), 2.61-2.73 (m, 4H), 2.56 (br t, J=6.5 Hz, 2H), 1.86-1.95 (m, 5H), 1.73-1.85 (m, 5H).

Step 1 of the Conversion of (S)-11 to Compound 1:

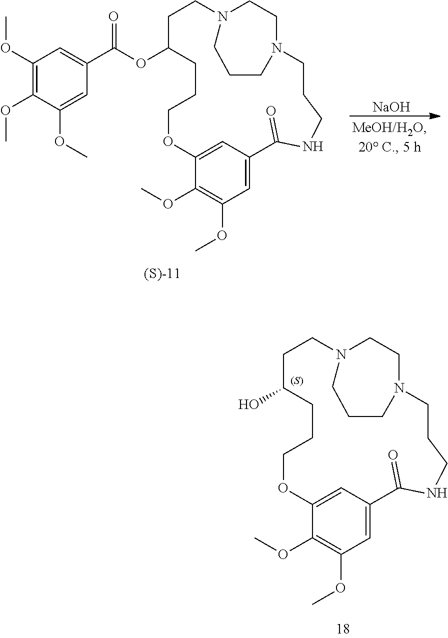

To a solution of compound (S)-11 (150 g, 238.19 mmol, 1 eq) in MeOH (800 mL) and H₂O (400 mL) was added NaOH (28.58 g, 714.58 mmol, 3 eq). The mixture was stirred at 20° C. for 5 hr. The solvent MeOH was removed under reduced pressure at 25° C. The mixture was diluted with H₂O (1500 mL) and extracted with DCM (500 mL×3). The organic layer was washed with brine, dried by Na₂SO₄. The solution was concentrated to afford compound 18 (116.5 g, crude) as yellow solid.

LCMS (ESI position ion) m/z: 436.2 (M+H)+ (calculated: 436.3)

¹H NMR (400 MHz, CD₃OD) δ 7.15 (dd, J=1.8, 7.6 Hz, 2H), 4.30-4.13 (m, 2H), 3.99-3.90 (m, 1H), 3.88 (s, 3H), 3.83 (s, 3H), 3.57-3.40 (m, 2H), 2.88-2.52 (m, 12H), 2.05-1.92 (m, 1H), 1.91-1.71 (m, 5H), 1.71-1.51 (m, 4H).

Step 2 of the Conversion of (S)-11 to Compound 1:

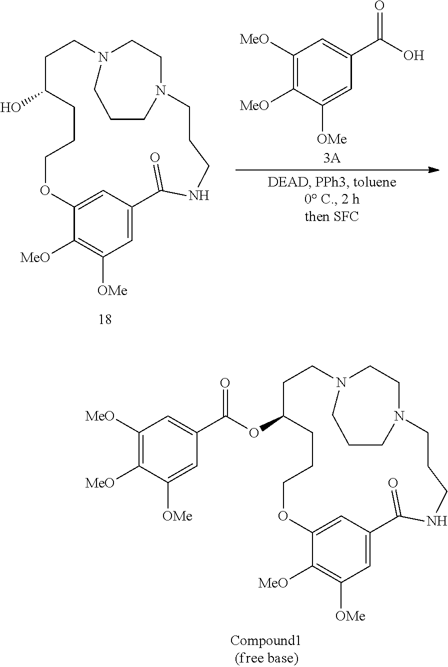 [SMILES: COc1cc2cc(c1OC)OCCC[C@H](O)CCN1CCCN(CCCNC2=O)CC1 ; COc1cc(C(=O)O)cc(OC)c1OC ; COc1cc(C(=O)O[C@@H]2CCCOc3cc(cc(OC)c3OC)C(=O)NCCCN3CCCN(CC2)CC3)cc(OC)c1OC]

A mixture of Compound 18 (10.00 g, 22.96 mmol, 1 eq), compound 3A (14.62 g, 68.88 mmol, 3 eq) and PPh₃ (30.11 g, 114.80 mmol, 5 eq) in toluene (250 mL) was added DEAD (19.99 g, 114.80 mmol, 20.87 mL, 5 eq) dropwise at 0° C. The mixture was stirred at 0° C. for 2 hr under nitrogen atmosphere. The reaction mixture was filtered by column chromatography (SiO₂, Petroleum ether/Ethyl acetate=1/0 to 0/1 and DCM/MeOH=10/1 to 1/1) to give the crude product. The crude product was purified by prep-HPLC (column: Welch Xtimate C18 250*50 mm*10 um; mobile phase: [water(FA)-ACN]; B %: 2%-32%, 15 min). The purified solution was concentrated and adjusted pH with NaHCO₃ to 7-8 at 0° C. The solution was extracted with DCM (500 mL×2). The organic layer was washed with brine, dried over Na₂SO₄. The solution was concentrated to afford Compound 1 (4.7 g, 33% yield) as a white solid. This reaction was carried out in 12 batches and totally affording 50 g of Compound 1 with an ee=60%. The compound was further purified by chiral SFC in the condition below to afford Compound 1 (35.5 g) as a white solid.
LCMS (ESI position ion) m/z: 630.2 (M+H)+ (calculated: 630.3), purity >99%
Chiral SFC: retention time=6,560 min, ee >99%

¹H NMR (400 MHz, CD₃OD) δ 7.31 (s, 2H), 7.20 (d, J=1.8 Hz, 1H), 7.13 (d, J=1.8 Hz, 1H), 5.49 (s, 1H), 4.31 (br d, J=8.3 Hz, 1H), 4.18 (br s, 1H), 3.85-3.89 (m, 9H), 3.81 (d, J=7.3 Hz, 6H), 3.56-3.66 (m, 1H), 3.38-3.49 (m, 1H), 2.97 (td, J=3.2, 10.3 Hz, 1H), 2.84-2.91 (m, 2H), 2.74-2.84 (m, 3H), 2.61-2.73 (m, 4H), 2.56 (br t, J=6.5 Hz, 2H), 1.86-1.95 (m, 5H), 1.73-1.85 (m, 5H), 1.83-1.72 (m, 5H).
Column: Chiralpak AD-3 50×4.6 mm I.D., 3 um
Mobile phase: Phase A for CO2, and Phase B for IPA (0.05% DEA);
Gradient elution: B in A from 5% to 40%
Flow rate: 3 mL/min; Detector: PDA
Column Temp: 35C; Back Pressure: 100 Bar
SFC: tR=9.658 min, 100% ee value

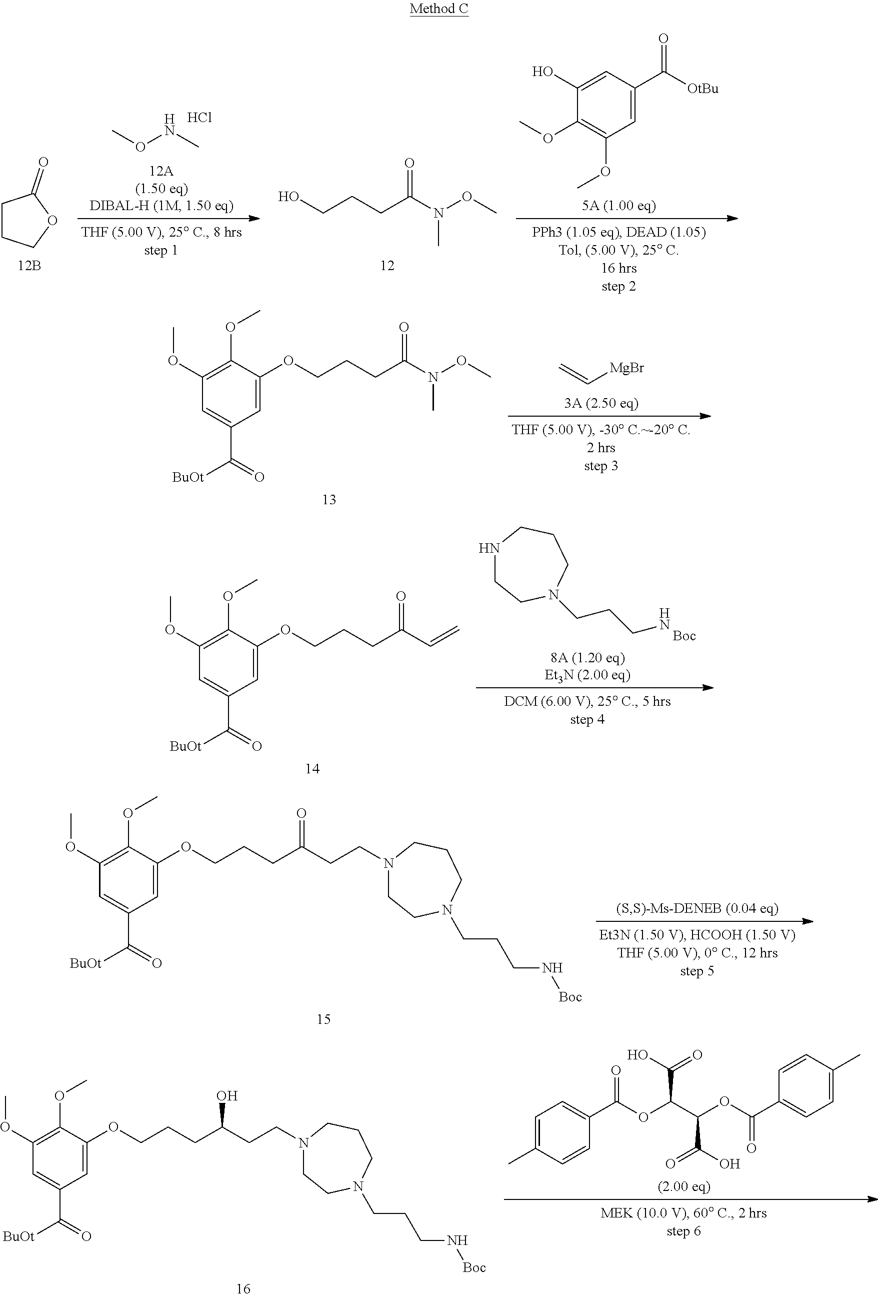

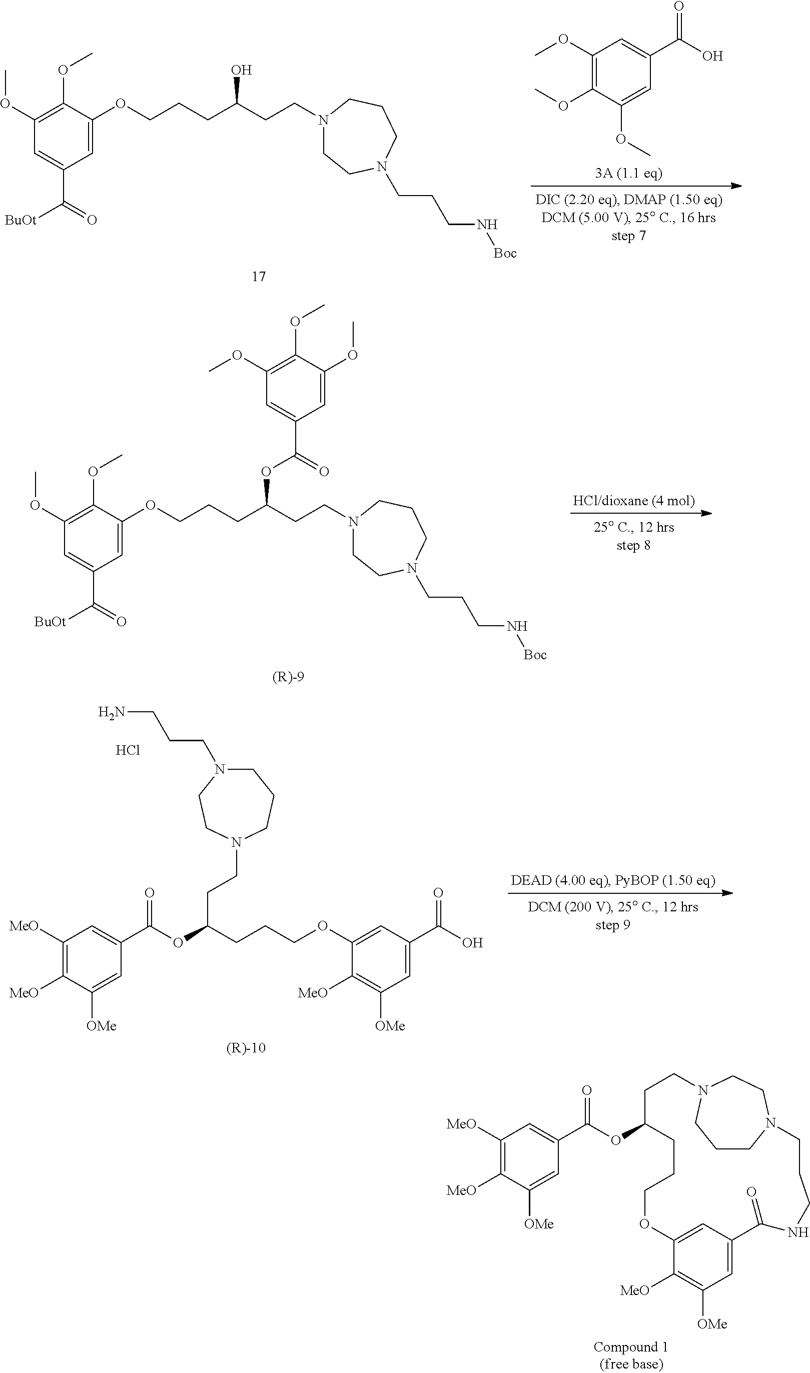

### Step 1:

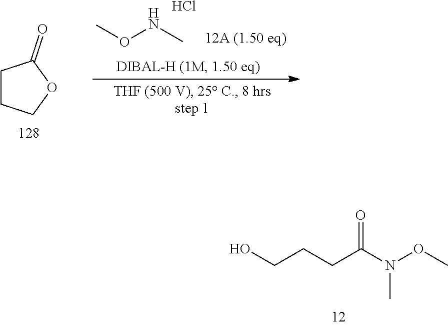

At 15-25° C., Compound 12A (210 g, 1.50 eq, 1.57 mol) was dissolved in THF (450 mL, 5.00 V). To the reaction mixture was added DIBAL-H (1.57 L, 1.50 eq, 1.57 mol) dropwise at 0-10° C. The reaction mixture was stirred at 15-25° C. for 2 hrs. The compound 12B (90.0, 1.00 eq, 1.05 mol) was added to the reaction mixture at 0-10° C. dropwise. The reaction mixture was stirred at 15-25° C. for 5 hrs. H₂O (63.0 mL) was added at 0-10° C. dropwise, then an aqueous solution of NaOH (15%, 63.0 mL) slowly, then additional H₂O (157 mL) at 0-10° C. slowly. The reaction mixture was stirred at 15-25° C. for 15 mins then dried over MgSO₄. The solvent was removed under reduced pressure to give the compound 12 (70.0 g, 0.48 mol, 45.5% yield) as yellow oil.

### Step 2:

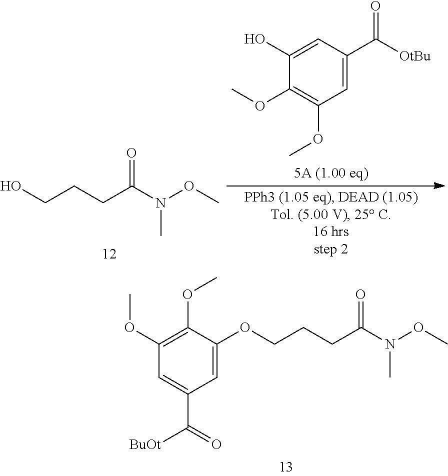

At 15-25° C., Compound 12 (30.0 g, 1.00 eq), compound 5A (51.8 g, 1.00 eq), PPh3 (56.1 g, 1.05 eq) and toluene (150 mL, 5.00 V) were charged into the reactor. DEAD (37.2 g, 1.05 eq) was added dropwise to the reaction mixture at 0-10° C. The reaction mixture was stirred at 15-25° C. for 24 hrs. The solvent was removed under reduced pressure and the residue was purified by silica column chromatography (SiO₂, Petroleum ether/Ethyl acetate=100/1 to 0/1) to give the compound 13 (50.0 g, 0.13 mol) as yellow solid.

¹H NMR (400 MHz, CDCl₃) δ 7.24 (s, 2H) 4.08-4.16 (m, 2H) 3.84-3.94 (m, 6H) 3.62-3.76 (m, 3H) 3.13-3.26 (m, 3H) 2.59-2.74 (m, 2H) 2.12-2.32 (m, 2H) 1.52-1.63 (m, 9H).

### Step 3:

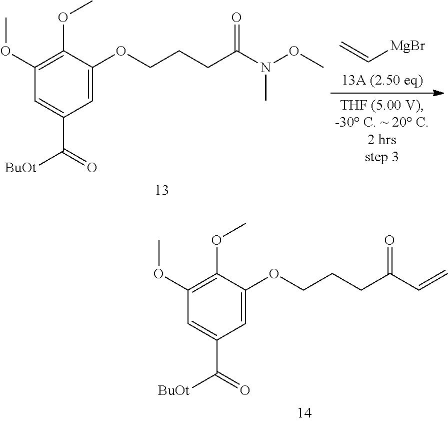

At 15-25° C., Compound 13 (45.0 g, 1.00 eq) was dissolved in THF (225 mL, 5.00 V). At −30° C., the compound 13A (293 mL, 1.00 eq, 1M) was added to the reaction mixture dropwise. The reaction mixture was stirred at −30° C. for 2 hrs. HCl (1.35 L, 1M in H₂O, 30.0 V) was slowly added-30° C. Ethyl acetate (225 mL, 5.00 V) was added. The organic phase was separated, washed with brine, dried over Na₂SO₄ and concentrated under reduced pressure to give the crude product. The crude product was purified by silica column chromatography (SiO₂, Petroleum ether/Ethyl acetate=100/1 to 0/1) to give the compound 14 (27.0 g, 75.5 mmol, 64.3% yield, 98.3% purity) as yellow oil.

¹H NMR (400 MHz, CDCl₃) δ 7.21-7.25 (m, 2H) 6.20-6.53 (m, 2H) 5.80-5.89 (m, 1H) 4.03-4.14 (m, 2H) 3.81-3.92 (m, 6H) 2.77-2.94 (m, 2H) 2.06-2.25 (m, 2H) 1.46-1.67 (m, 10H).

### Step 4:

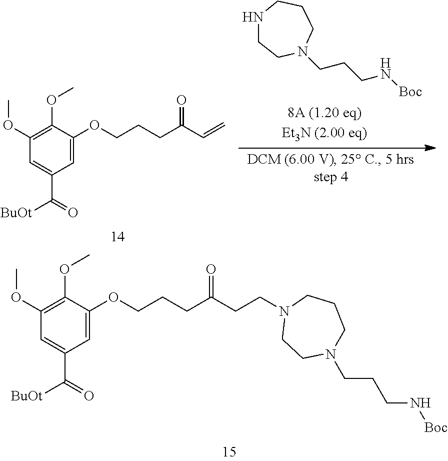

At 15-25° C., Compound 14 (27.0 g, 1.00 eq) was dissolved in DCM (135 mL, 5.00 V). The compound 8A (24.7 g, 1.00 eq) and Et₃N (15.6 g, 2.00 eq) was added and the reaction mixture was stirred for 12 hrs. The reaction mixture was concentrated to give the crude compound 15 (42.0 g, 69.1 mmol) as a yellow oil.

¹H NMR (400 MHz, CD₃OD) δ 7.21-7.28 (m, 2H) 4.01-4.13 (m, 2H) 3.75-3.91 (m, 6H) 2.99-3.16 (m, 3H) 2.63-2.85 (m, 13H) 2.44-2.58 (m, 3H) 2.02-2.12 (m, 2H) 1.75-1.86 (m, 2H) 1.61-1.66 (m, 2H) 1.59 (s, 9H) 1.43 (s, 9H).

### Step 5:

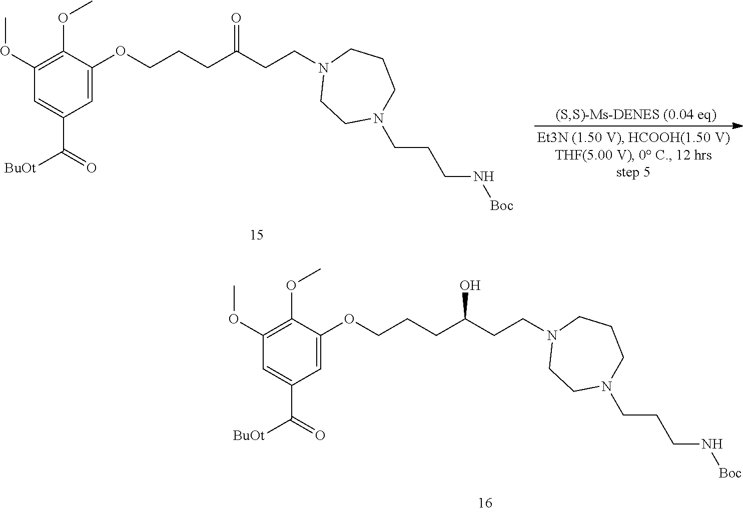 [1292] At 15-25° C., to a solution of Compound 15 (3.00 g, 1.00 eq), HCOOH/Et3N (9.00 mL, 1:1, 3.00 V) in THF (15.0 mL, 5.00 V) was added (S,S)-Ms-DENEB catalyst (0.11 g, 0.04 eq). The reaction mixture was stirred at 15-25° C. for 12 hrs. H₂O (9.00 mL, 3.00 V) and DCM (9.00 mL, 3.00 V) was added to the reaction mixture. The organic phase was separated, washed with brine, dried over Na₂SO₄ and concentrated under reduced pressure to give the crude product. The crude product was purified by silica column chromatography (SiO₂, Petroleum ether/Ethyl acetate=100/1 to 0/1) to give the compound 16 (1.80 g, 2.89 mmol, 98.1% purity) as yellow oil.
LCMS (ESI position ion) m/z: 630.2 (M+H)+ (calculated: 630.3)
Chiral HPLC: retention time=21.398 and 23.972 min, ee=85.9%

### Step 6:

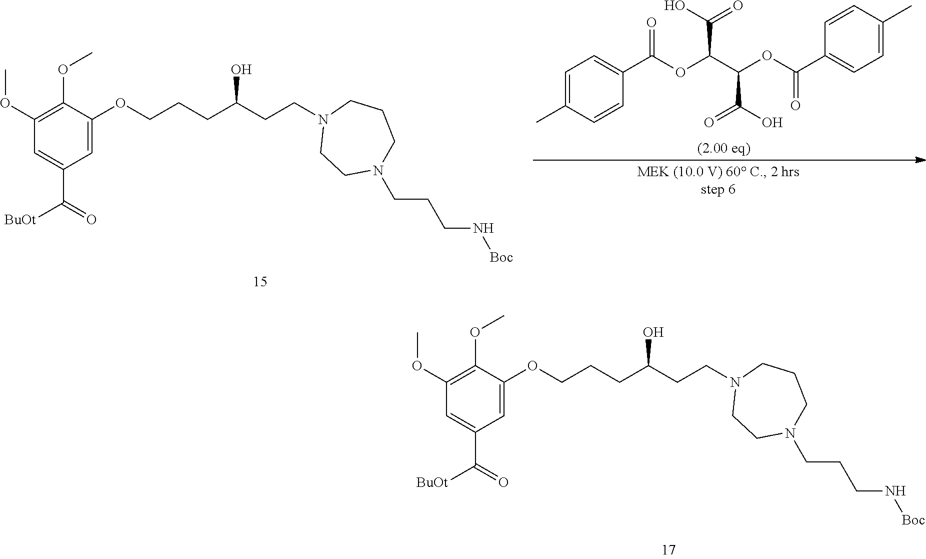

Compound 16 (1.00 eq) was added to 2-butanone (MEK) (5.00 V) at 15-25° C. then the mixture was stirred at 55-60° C. for 2 hrs. Di-p-toluoyl-1-tartaric acid (2.00 eq) was added and the mixture stirred at 10-20° C. for 12 hrs.

The mixture was concentrated to give the crude product. (Reddish brown solid) The solid was triturated with 2-butanone (10.0 V) at 25° C. for 30 mins. (white solid). The mixture was filtered and the filter cake was washed twice with 2-butanone (1.00 V). The solid was dissolved in water (3.00 V). A saturated solution sodium carbonate was added into the mixture to adjust pH=11. DCM (3.00 V) was added into the mixture and the organic phase was separated, washed with brine, dried over Na₂SO₄, concentrated under reduced pressure to give the compound 17 as a reddish brown oil.
Chiral HPLC: retention time=18.335 and 20.673 min, ee=95.9%

### Step 7:

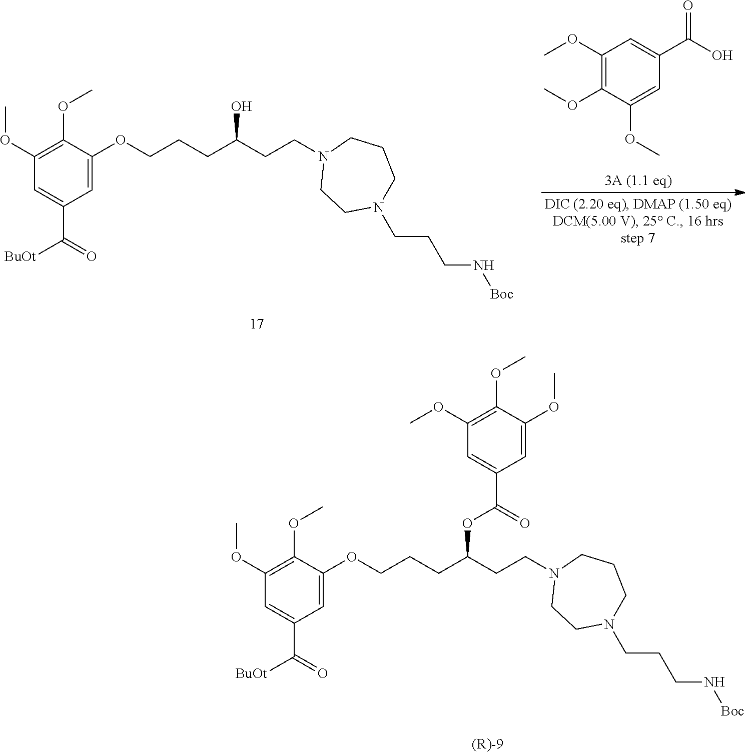

At 15-25° C., to a solution of Compound 17 (1.00 g, 1.00 eq), compound 3 A (1.20 eq) in DCM (5.00 V) was added DIC (2.20 eq) and DMAP (1.50 eq). The reaction mixture was stirred at 15-25° C. for 16 hrs. H₂O (3.00 V) and DCM (3.00 V) was added and the organic phase was separated, washed with brine, dried over Na₂SO₄ and concentrated under reduced pressure to give the crude product. The crude product was purified by silica column chromatography (SiO₂, Petroleum ether/Ethyl acetate=100/1 to 0/1) to give the compound (R)-9 (600 mg, 45% yield).

LCMS (ESI position ion) m/z: 804.4 (M+H)+ (calculated: 804.5)

### Step 8:

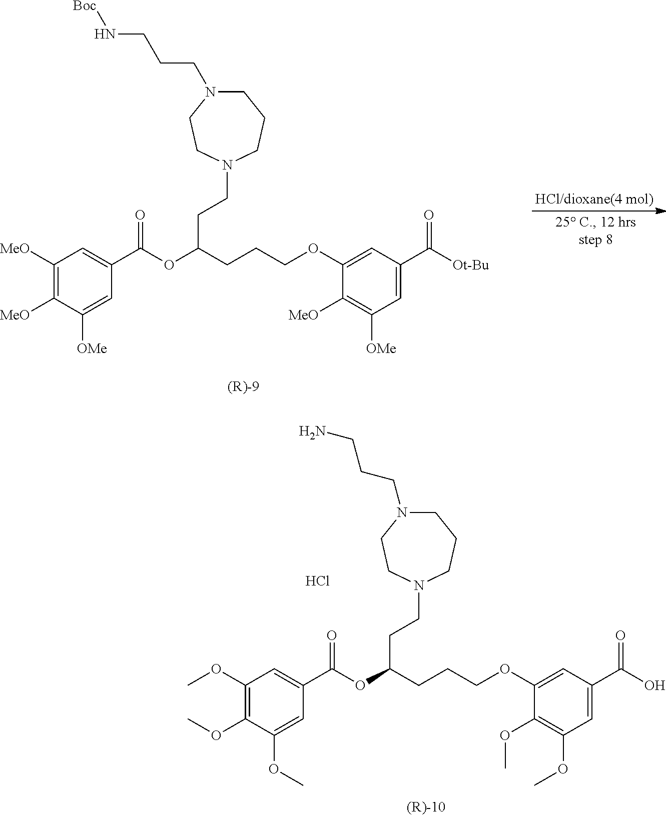 [SMILES: COc1cc(C(=O)OC(CCCOc2cc(C(=O)OC(C)(C)C)cc(OC)c2OC)CCN2CCCN(CCCNC(=O)OC(C)(C)C)CC2)cc(OC)c1OC ; COc1cc(C(=O)O[C@H](CCCOc2cc(C(=O)O)cc(OC)c2OC)CCN2CCCN(CCCN)CC2)cc(OC)c1OC ; Cl]

At 0-5° C., a solution of HCl in dioxane (4 mol, 7.60 L) and compound (R)-9 (1086 g, 1.35 mol, 1.00 eq) was charged into a 20.0 L reactor. The reaction mixture was stirred at 25° C. for 12 hrs. The solvent was removed under reduced pressure to give the compound (R)-10 (1050 g, as HCl Salt) as yellow solid. Purity determined by quantitative NMR: 75.2%.

¹H NMR (400 MHz, CD₃OD) δ 7.29 (s, 2H), 7.26 (s, 2H) 5.2-5.37 (m, 1H), 4.11 (s, 2H), 3.94 (brs, 4H), 3.78-3.90 (m, 15H), 3.33-3.45 (m, 4H), 3.08 (t, J=7.6 Hz, 2H), 2.12-2.49 (m, 6H), 1.90-2.09 (m, 4H).

### Step 9:

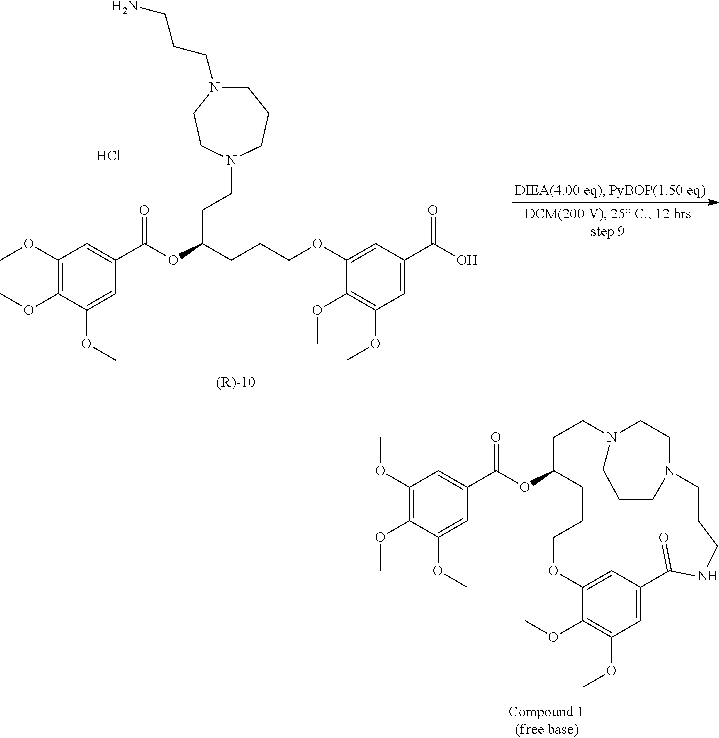 [SMILES: COc1cc(C(=O)O[C@H](CCCOc2cc(C(=O)O)cc(OC)c2OC)CCN2CCCN(CCCN)CC2)cc(OC)c1OC ; Cl ; COc1cc(C(=O)O[C@@H]2CCCOc3cc(cc(OC)c3OC)C(=O)NCCCN3CCCN(CC2)CC3)cc(OC)c1OC] [1306] At 20° C., compound (R)-10 (1050 g, 1.53 mol, 1.00 eq, HCl) and DCM (210 L) were charged into the reactor. Then, DIEA (793 g, 6.13 mol, 4.00 eq) and PyBOP (38.4 g, 2.29 mol, 1.50 eq) was added to the reactor at 20° C. The reaction mixture was stirred at 25° C. for 12 hrs. The reaction mixture was concentrated at 35-40° C. to give the residue. The residue was triturated with MeOH (4.2 L, 4.00 V) at 20° C. for 60 min. The mixture was filtered and concentrated in vacuum to give Compound 1 (470 g, 34.94 mmol, 48.6% yield) as white solid. Purity determined by quantitative NMR: 75.2%

¹H NMR (400 MHz, CD₃OD) δ 7.31 (s, 2H), 7.20 (d, J=1.8 Hz, 1H), 7.13 (d, J=1.8 Hz, 1H), 5.49 (s, 1H), 4.31 (br d, J=8.3 Hz, 1H), 4.18 (br s, 1H), 3.85-3.89 (m, 9H), 3.81 (d, J=7.3 Hz, 6H), 3.56-3.66 (m, 1H), 3.38-3.49 (m, 1H), 2.97 (td, J=3.2, 10.3 Hz, 1H), 2.84-2.91 (m, 2H), 2.74-2.84 (m, 3H), 2.61-2.73 (m, 4H), 2.56 (br t, J=6.5 Hz, 2H), 1.86-1.95 (m, 5H), 1.73-1.85 (m, 5H).

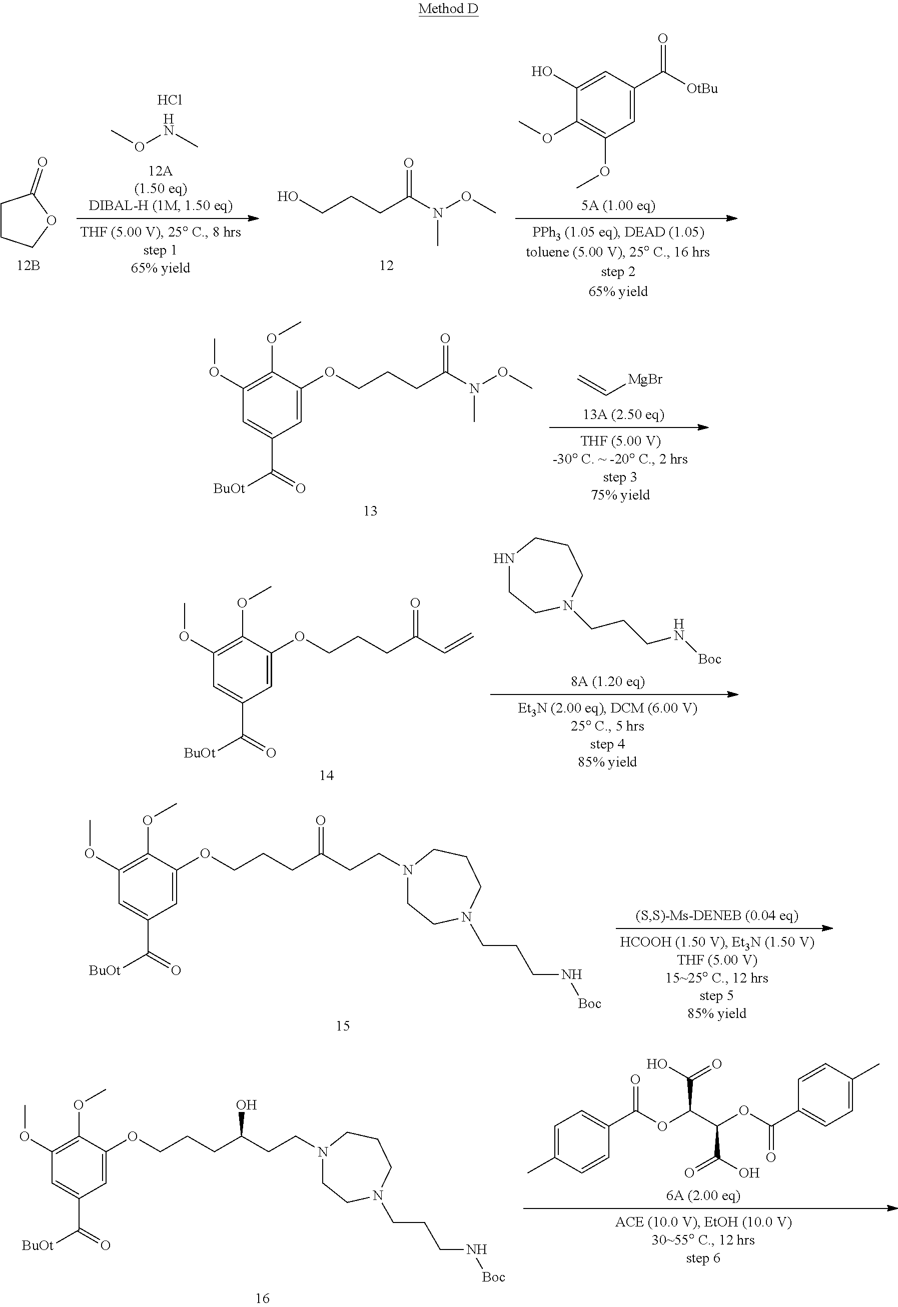

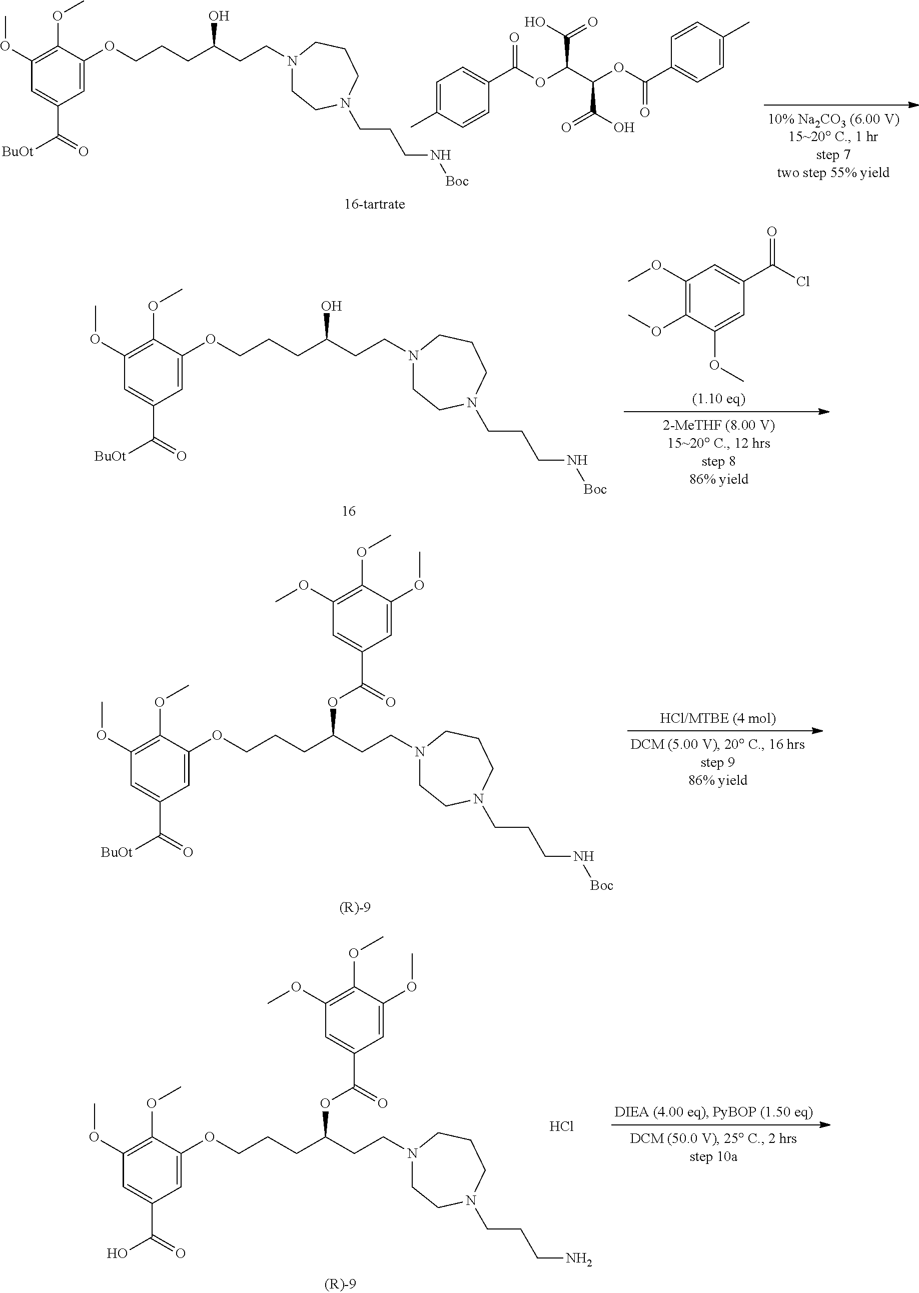

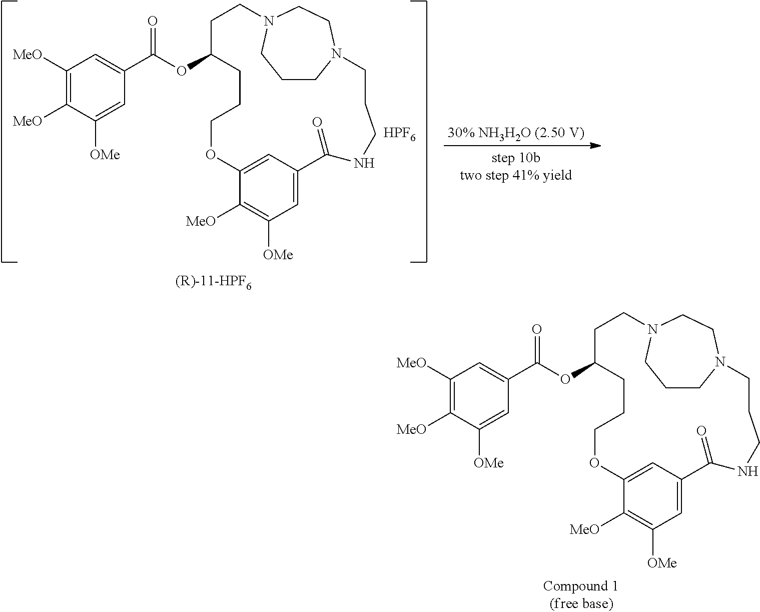

### Step 1:

Compound 12A (1.70 kg, 1.50 eq) was dissolved in THF (5.00 L, 5.00 V). To the reaction mixture was added DIBAL-H (17.4 L, 1 M in toluene, 1.50 eq) dropwise at 0-10° C. The reaction mixture was stirred at 20-30° C. for 2 hrs. The compound 12B (1.00 kg, 1.00 eq) was added to the reaction mixture at 0-10° C. dropwise. The reaction mixture was stirred at 20-30° C. for 12 hrs. H₂O (700 mL, 0.04× mL) was added at 0-10° C. dropwise, then an aqueous solution of NaOH (700 mL, 0.04× mL, 15%) slowly, then additional H₂O (1.74 L, 0.1×mL) at 0-10° C. slowly. The reaction mixture was stirred at 20-30° C. for 15 mins then dried over MgSO₄ (500 g). The solvent was removed under reduced pressure to give the compound 12 (7.80 kg, 65% yield) as yellow oil.

¹H NMR: (400 MHz, CDCl₃) δ ppm 1.75-1.95 (m, 2H), 2.53-2.63 (m, 2H), 3.13-3.24 (m, 3H), 3.61-3.72 (m, 5H).

### Step 2:

At 15-25° C., Compound 12 (7.80 kg, 1.00 eq), compound 5A (8.30 kg, 1.00 eq), PPh3 (9.00 kg, 1.05 eq) and toluene (35 L) were charged into the reactor. DEAD (13.0 kg, 1.05 eq) was added slowly to the reaction mixture at 0-10° C. The reaction mixture was stirred at 15-25° C. for 12 hrs. The reaction system was filtered, and the filter cake was washed with MTBE. Water (0.3 L) followed by MgCl₂ (5.64 kg) was added to the filtrate, and the mixture was stirred 20-30° C. for 2 hrs. The reaction system was filtered, and the filter cake was washed with MTBE. The filtrate was washed with 10% citric acid aqueous solution (25.0 L, 3.00× by volume). The organic phase was washed with 5% brine and dried over Na₂SO₄ (4.15 kg, 0.50× by weight). The organic phase was concentrated at 45-55° C. to a volume of 12-20 L. n-Heptane (12.5 L, 1.50× by volume) was added, and the system was reduced to 12-20 L; this was repeated. n-Heptane (41.5 L, 5.00× by volume) was added, and the system was heated at 50-60° C. for 2 hrs with stirring. The system was cooled, filtered and the cake was washed with n-heptane. The filter caked was vacuum dried at 40-50° C., resulting in compound 13 (6.50 kg, 98% purity by HPLC, 65% yield).

¹H NMR (400 MHz, CDCl₃) δ ppm 7.22-7.26 (m, 2H), 4.08-4.15 (m, 2H), 3.86-3.92 (m, 6H), 3.67-3.71 (m, 3H), 3.16-3.24 (m, 3H), 2.63-2.73 (m, 2H), 2.13-2.25 (m, 2H), 1.57-1.60 (m, 9H).

### Step 3:

At 15-25° C., Compound 13 (6.00 kg, 1.00 eq) was dissolved in THF (30.0 L, 5.00 V). At −20° C., compound 13A (39.0 L, 1 M in THF, 6.52× by volume) was added to the reaction mixture slowly. The reaction mixture was stirred at −10-0° C. for 2 hrs. HCl (30.0 L, 1M, 5.00× by volume) was slowly added controlling the pH at 1˜3 at 0˜20° C. MTBE (18.0 L, 3.00× by volume) was added. The organic phase was separated and washed with 0.5 N HCl (18.0 L, 3.00× by volume) twice. The organic phase was washed with 5% NaHCO₃ aqueous (18.0 L, 3.00× by volume), 5% brine (18.0 L, 3.00× by volume), dried over Na₂SO₄, filtered, and concentrated under reduced pressure to give compound 14 (4.50 kg, 75% yield, 92.3% purity) as yellow oil.

¹H NMR (400 MHz, CDCl₃) δ ppm 7.20-7.26 (m, 2H), 6.20-6.51 (m, 2H), 5.78-5.93 (m, 1H), 4.03-4.17 (m, 2H), 2.80-2.91 (m, 2H), 2.11-2.23 (m, 2H), 1.53-1.64 (m, 9H).

### Step 4:

At 15-25° C., Compound 14 (5.20 kg, 1.00 eq) was dissolved in DCM (26.0 L, 5.00× by volume). The compound 8A (4.57 kg, 1.00 eq) and Et3N (3.0 kg, 2.00 eq) was added and the reaction mixture was stirred for 12 hrs. The reaction mixture was concentrated to give the crude compound 15 (7.30 kg, 90.7% purity 85% yield) as a yellow oil.

¹H NMR (400 MHz, CDCl₃) δ ppm 7.17-7.24 (m, 2H), 5.63-5.79 (m, 1H), 4.00-4.07 (m, 2H), 3.81-3.89 (m, 6H), 3.11-3.21 (m, 2H), 2.76-2.84 (m, 2H), 2.44-2.70 (m, 15H), 2.04-2.14 (m, 2H), 1.71-1.79 (m, 2H), 1.57 (s, 9H), 1.41 (s, 9H).

### Step 5:

At 15-25° C., to a solution of compound 15 (5.00 kg, 1.00 eq), HCOOH (7.50 L, 1.50× by volume), Et3N (7.50 L, 1.50× by volume) in THF (25.0 L, 5.00× by volume) was added (S,S)-Ms-DENEB catalyst (360 g, 0.04 eq). The reaction mixture was stirred at 10-15° C. for 12 hrs. The reaction was cooled to 5-10° C., and pH of the system was adjusted to 11˜12 with saturated Na₂CO₃ aqueous solution (almost 15.0 L). DCM (15.0 L, 3.00 V) was added to the reaction mixture. The organic phase was separated, washed with brine, dried over Na₂SO₄ and concentrated under reduced pressure to give compound 16 (4.50 kg, 83.4% purity, 84.9% ee, 85% yield) as brown oil.

| Instrument: | Shimadzu 20AD | | | | | |
|---|---|---|---|---|---|---|
| Column: | Gemini-NX C18 4.6*150 mm, 5 um | | | | | |
| Column temperature: | 40° C. | | | | | |
| Mobile phase A(MPA) | H₂O + 0.04%(v/v) TFA | | | | | |
| Mobile phase B(MPB) | ACN + 0.02%(v/v) TFA | | | | | |
| Flow rate: | 1.2 mL/min | | | | | |
| Gradient Ratio: | Time(min) | 0.01 | 16 | 19 | 19.01 | 20.00 |
| | MPA(%) | 90 | 20 | 0 | 90 | 90 |
| | MPB(%) | 10 | 80 | 100 | 10 | 10 |

| | |
|---|---|
| Detection: | 220 nm 215 nm 254 nm |

### Steps 6-7:

The compound 16 (62.0 kg, 1.00× by weight) was dissolved in acetone (ACE) (434 L, 7.00× by volume) and EtOH (434 L, 7.00× by volume) at 10-20° C. then the mixture was stirred at 50-55° C. for 1 hr and then allowed to cool to 25-30° C. Acetone (186 L, 3.00× by volume), ethanol (186 L, 3.00× by volume) and compound 6A (78.5 kg, 1.26× by weight) were added and the mixture was stirred at 50˜55° C. for 1 hr. The system was cooled to 25˜30° C. at a rate of 3˜5° C. degrees an hour. The mixture was filtered, and the filter cake was washed with ACE:EtOH=1:1 (4.50 L, 1.00× by volume) and dried under N₂ in a blast drying oven at 45˜55° C. affording the product 16-tartrate (5.10 kg, 98.0% purity, 97.5% ee). The salt Compound 16-tartrate can be converted to the corresponding acid by means known in the art, such as those in the Scheme above.

### Step 8:

Compound 16 (1.50 kg, 1.0 equiv.) was dissolved in 2-MeTHF (20.2 L). Under N₂, the solution was cooled to 10-15° C. A solution of 2, 3, 4 trimethoxybenzoyl chloride (624.0 g, 1.10 equiv) in 2-MeTHF (3.00 L) was added to the solution dropwise. The reaction was stirred at 15-20° C. for 16 h. At which time, aqueous Na₂CO₃ (10%, 4.5 L) was added to adjust the pH to 11-12 at 10-20° C. The organic phase was separated, washed with 10% NaCl (4.5 L), dried over Na₂SO₄, and filtered. The solvent of the filtrate was removed under reduced pressure to give compound (R)-9 (1.70 kg, 94.4% purity, 97.7% ee, 86% yield).

### Step 9:

Compound (R)-9 (200 g, 1.00 equiv) was dissolved in DCM (1.00 L) under N₂. At 15-20° C., 4M HCl in MTBE (600 mL) was added. The reaction mixture was stirred at 15-20° C. for 16 hrs. MTBE (2.00 L) was added dropwise over 20 min. A white precipitate formed. Stirring ceased, and the mixture was allowed to stand for 30 min. The supernatant liquid was removed with a peristalic pump, reducing the solution to a volume of ˜1.0 L. The mixture was filtered, and the filter cake was dried under vacuum at 40-45° C. to afford compound (R)-10 (150 g, 98.6 purity, 86% yield).

Steps 10a-10b:

PyBOP (913 g, 1.5 eq) was dissolved in DCM (40.0 L, 30.0 V) under N₂ at 15˜25° C. DIEA (600 g, 4.00 eq) was added followed by a solution of Compound (R)-10 (800 g, 1.00 eq) in DCM (1.60 L, 20.0 V) over about 1.5 hrs. The mixture was stirred for an additional 20 min at 15˜25° C. At which time, the reaction was concentrated to ˜2.00 V at 40-45°. The solution was washed with water (2.40 L, 3.00 V) three times. The organic phase was washed with 10% NaCl (2.40 L, 3.00 V). The organic phase was dried over Na₂SO₄ (200 g, 0.25× by weight), and filtered. The filter cake was washed with MeOH. MeOH (2.40 L, 3.00 V) was added into the filtrate and then the mixture was concentrated to about 2.00 V. The addition of MeOH and concentration was repeated two more times to remove any residual DCM. MeOH (2.40 L, 3.00 V) was added into mixture and stirred at 15˜25° C. for 12 hrs. The system was filtered and the resultant cake was washed with MeOH (0.80 L, 1.00 V). The filter cake was dried cake under vacuum at 45˜50° C. and 550 g of Compound 1 —HPF₆ was obtained with 98.8% purity (66% yield).

A reaction vessel was charged with MeOH (4.00 L, 5.00 V) and 550 g of Compound 1 HPF₆. The reaction vessel was subsequently charged with 30% of NH₃·H₂O (1375 mL, 2.50 V) slowly at 15˜25° C. for 20 mins until the system gradually became clear. The system was extracted with DCM three times (2750 mL, (5.00 V)×3). The organic layers were combined, washed with 10% of Na₂CO₃ (1.50 L, 3.00 V) one time, and washed with 10% NaCl (1.50 L, 3.00 V). The organic layer was dried over Na₂SO₄ (125 g, 0.25× by weight), filtered (washing the filter cake with DCM (250 mL, 0.50 V)) and solvent was removed under vacuum to give 410 g of crude Compound 1.

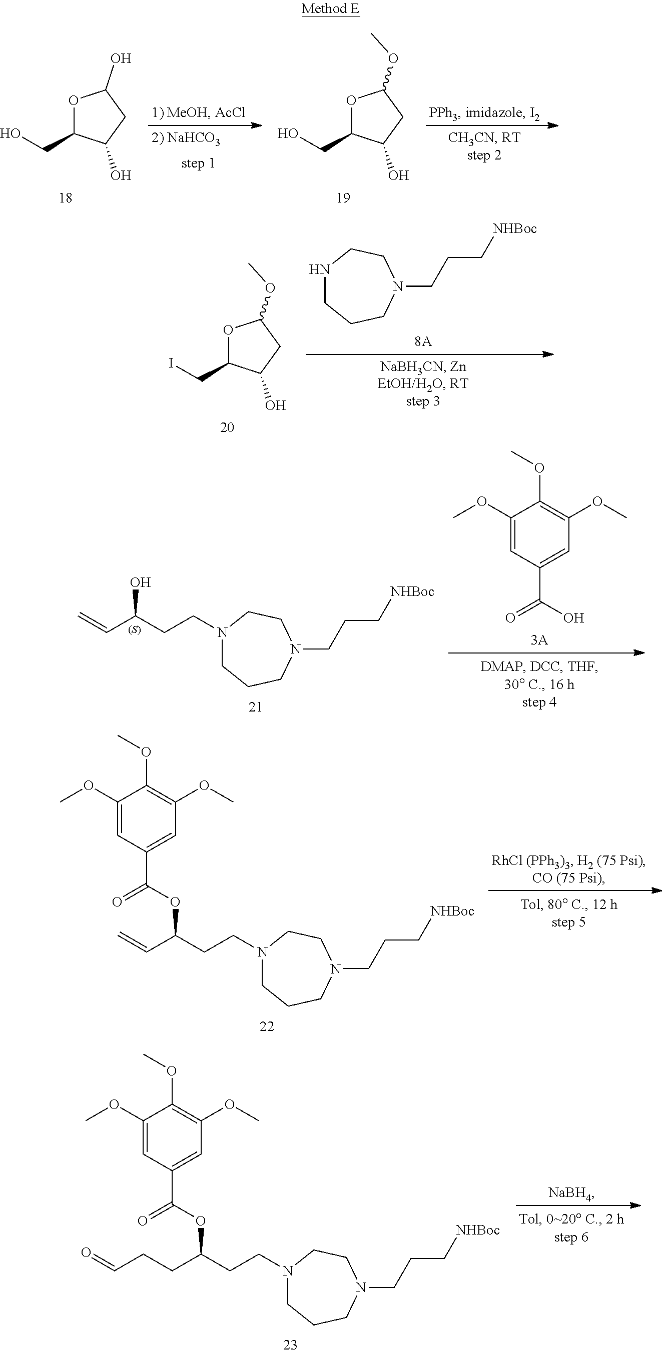

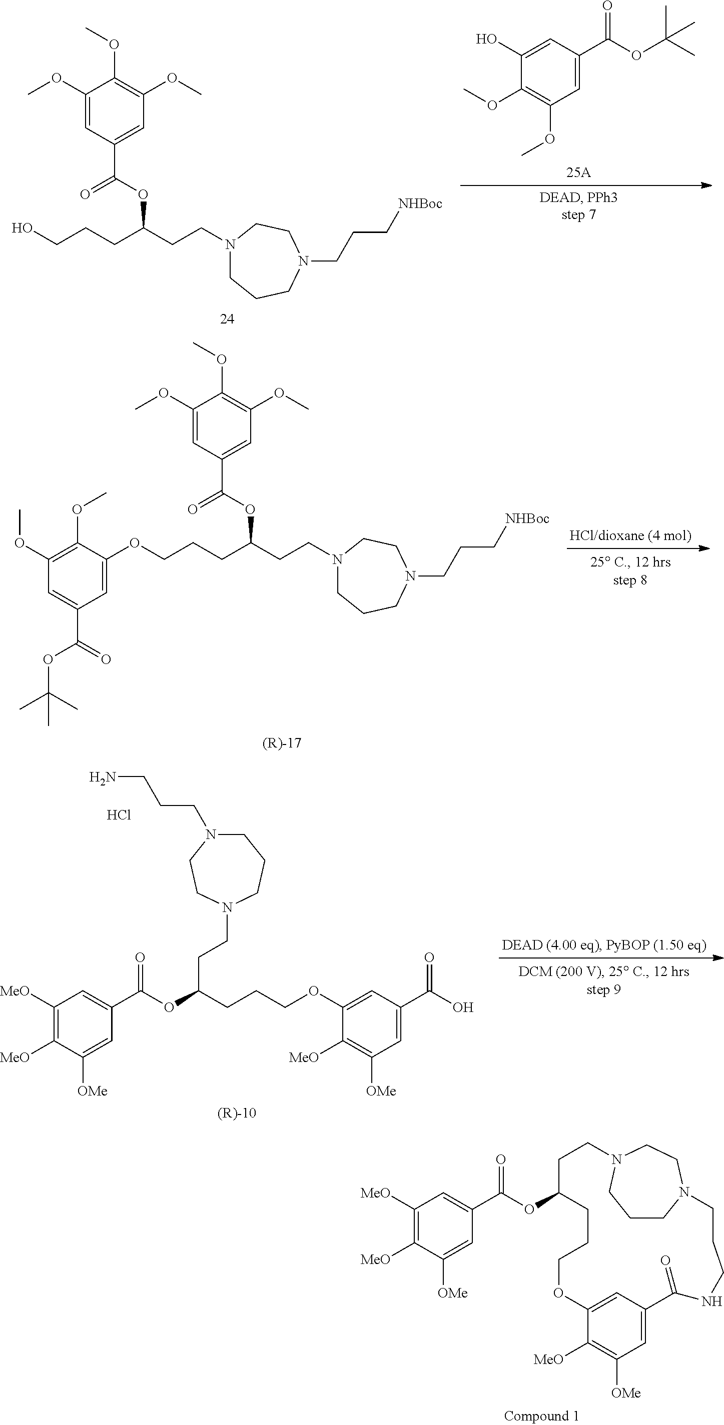 [SMILES: COc1cc(C(=O)O[C@H](CCCO)CCN2CCCN(CCCNC(=O)OC(C)(C)C)CC2)cc(OC)c1OC ; COc1cc(C(=O)OC(C)(C)C)cc(O)c1OC ; COc1cc(C(=O)O[C@H](CCCOc2cc(C(=O)OC(C)(C)C)cc(OC)c2OC)CCN2CCCN(CCCNC(=O)OC(C)(C)C)CC2)cc(OC)c1OC ; COc1cc(C(=O)O[C@H](CCCOc2cc(C(=O)O)cc(OC)c2OC)CCN2CCCN(CCCN)CC2)cc(OC)c1OC ; Cl ; COc1cc(C(=O)O[C@@H]2CCCOc3cc(cc(OC)c3OC)C(=O)NCCCN3CCCN(CC2)CC3)cc(OC)c1OC]

Example 11-Crystalline Form 1 Compound 1 (free base) and Crystalline Form 2 Compound 1 (free base): Approximately 100 mg of amorphous Compound 1 (free base) was weighed into a 1.5 mL screw-cap vial. Acetonitrile was added in 100 μL aliquots while stirring at 40° C. until the material dissolved (500 μL). The solution was stirred at 40° C. for 0.25 hr and then cooled to 5° C. at 0.1° C./min. A thick slurry (poorly mixed) was obtained at ca. 30° C. and so 200 μL of tBME was added as anti-solvent, but the slurry dissolved. After 11 hr at 5° C., a slurry was obtained. A portion of the slurry was isolated by centrifugation (0.22 μm nylon filter) and analyzed by XRPD. The isolated solids were consistent with a mixture of crystalline Form 1 Compound 1 (free base) and crystalline Form 2 Compound 1 (free base), which dried to crystalline Form 1 Compound 1 (free base).

Further aliquots of tBME were added to the remaining slurry (total of 600 μL), but dissolution was observed. Aliquots of heptane (3×100 μL) were added, but solution remained. Material was recovered by rotary evaporation and re-slurried in acetonitrile, then isolated by centrifugation (0.22 μm nylon filter) and analyzed by XRPD and found to be crystalline Form 1 Compound 1 (free base).

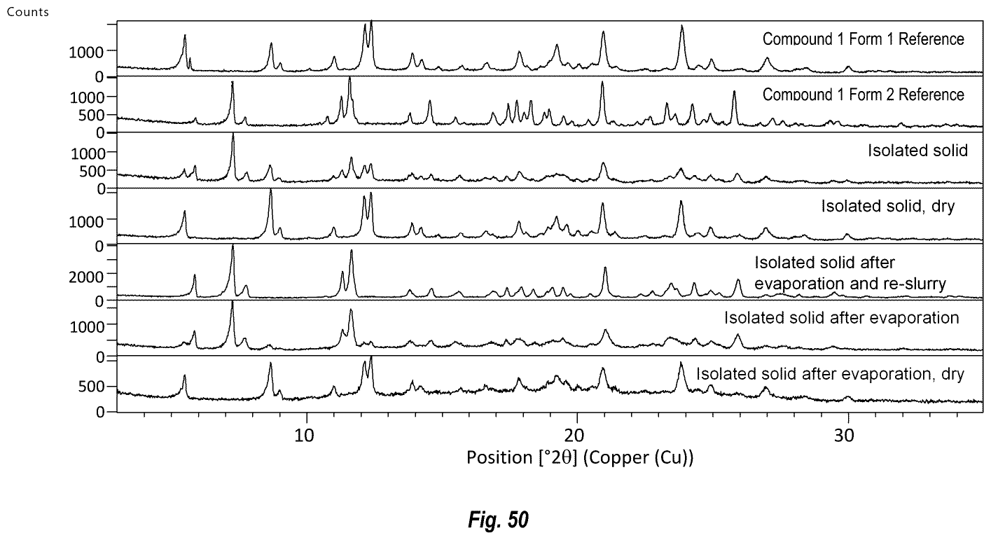

All liquors were evaporated to recover material. Solids were dried under vacuum at 35° C. for ca. 21 hr and then re-analyzed by XRPD. The solids were found to be crystalline Form 2 Compound 1 (free base), which dried to crystalline Form 1 Compound 1 (free base) with some loss of crystallinity. FIG. 50 is a stackplot of XRPD patterns taken during this experiment including crystalline Form 1 Compound 1 (free base) and crystalline Form 2 Compound 1 (free base) reference patterns. FIG. 51 is a separate XRPD pattern of crystalline Form 1 Compound 1 (free base) and FIG. 52 is a separate XRPD Pattern of crystalline Form 2 Compound 1 (free base) each prepared separately. FIG. 51 corresponds to FIG. 58 in U.S. Provisional Application No. 63/491,684. That original figure (FIG. 58) is believed to contain an artifact at 6.5° 2θ because the peak shape was usually narrow and the peak was not present in other patterns of the same compound. Accordingly, that peak has been removed in the current figure (FIG. 51).

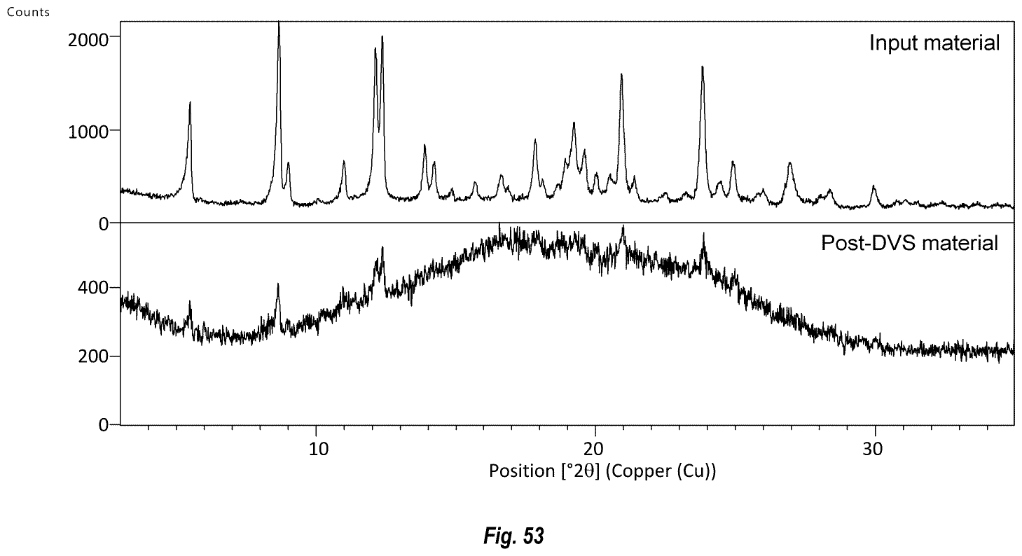

Hot stage microscopy showed that crystalline Form 1 Compound 1 (free base) started to melt at 65° C., with melting complete at 80° C. DVS analysis indicated that crystalline Form 1 Compound 1 (free base) was slightly hygroscopic with water uptake of 0.9% between 40-80% RH in the first sorption cycle. A further uptake of 3.8% between 80-90% RH likely resulted in formation of amorphous material, which was then moderately hygroscopic (uptake of 3.3% at 80% RH) in the second sorption cycle. Post-DVS XRPD analysis indicated that predominantly amorphous material was recovered (traces of crystalline Form 1 Compound 1 (free base)) as shown in FIG. 53.

Example 12-Stability Studies of Amorphous Compound 1 (free base) and Crystalline Form 1 Compound 1 (free base): Seven-day stability studies were carried out comparing amorphous Compound 1 (free base) and crystalline Form 1 Compound 1 (free base) at 40° C./75% RH, under ambient conditions and at 80° C. After storage at 40° C./75% RH and 80° C., the amorphous material formed glassy solids which were pale yellow (40° C./75% RH) or orange (80° C.). It is likely that the material melted and re-solidified at 80° C. and deliquesced at 40° C./75% RH. HPLC analysis showed that no significant degradation was observed after storage of the amorphous material at 40° C./75% RH and ambient conditions, but that the material degraded after storage at 80° C. (decrease in purity of ca. 11% area). XRPD analysis showed that the amorphous material remained amorphous under all storage conditions.

After storage at 40° C./75% RH and 80° C., crystalline Form 1 Compound 1 (free base) formed glassy solids which were colorless (40° C./75% RH) or orange (80° C.). It is likely that the material melted and resolidified at 80° C. and deliquesced at 40° C./75% RH. HPLC analysis showed that no significant degradation was observed after storage of crystalline Form 1 Compound 1 (free base) at 40° C./75% RH and ambient conditions, but that the material degraded after storage at 80° C. (likely decrease in purity of ca. 15% area). XRPD analysis showed that crystalline Form 1 Compound 1 (free base) was retained after storage at ambient conditions, but amorphous material was recovered from storage at 40° C./75% RH and 80° C. Table 48 below summarizes the results.

TABLE 48
Results of stability studies

| Input Material | | | | | Solid | |
|---|---|---|---|---|---|---|
| | Purity/ | | Storage | Post-Storage | Purity/ | XRPD |
| Material | % area | Appearance | Conditions | Observations | % area | Analysis |
| Amorphous | 98.8 | White solid | 40° C./75% | Pale yellow | 98.8 | A |
| Compound | | (NE12) | RH | glassy solid | | |
| 1 | | | Ambient | White solid | 98.9 | A |
| | | | 80° C. | Orange glassy | 87.5 | A |
| | | | | solid | | |
| Form 1 | Not | White solid | 40° C./75% | Glassy solid | 99.7 | A |
| Compound | determined | (NE12) | RH | | | |
| 1 | | | Ambient | White solid | 99.9 | A |
| | | | 80° C. | Orange glassy | 84.1 | 1 |
| | | | | solid | | |
| | | | 40° C./75% | Pale yellow | 98.8 | A |
| | | | RH | glassy solid | | |

A = Amorphous Compound 1 (free base)
1 = Crystalline Form 1 Compound 1 (free base)
NE12 is a color code from the Signa Aldrich colour chart

Example 13-Amorphous Compound 1 mono(hydrogen sulfate) and Crystalline Form 10 Compound 1 mono(hydrogen sulfate): Approximately 25 mg of amorphous Compound 1 (free base) was weighed out into 1.5 mL screw-cap vials and a stirrer bar was added. 100 μL of each solvent listed in Table 49 below was added to the free base. The samples were stirred at 40° C. for 5-10 min and then 1 mol. equiv. of sulfuric acid was added to the free base, rinsing out the transfer vial with a further 50 μL of solvent. Prior to acid addition, complete dissolution of the free base was observed in all the solvent systems assessed except for acetonitrile where partial dissolution was observed.

Experiments were temperature cycled between 40° C. and 5° C. (1 hour hold; 0.1° C./min ramp/cool) for ˜24-68 hours. Anti-solvent addition (ASA) was then carried out on slurries at 5° C. and at 30° C. or 40° C. where clear solutions or gums were observed. Experiments were then heated to 30° C. for 1 hour. Temperature cycling was then continued for ˜16-69 hours, followed by further ASA and temperature cycling if required.

Materials were isolated at 5° C. and analyzed three times using XRPD. First, an XRPD was obtained immediately following isolation from the solvent system (“Damp XRPD”). Then, the material was dried under vacuum at ˜40° C. for ˜17-91 hours and then reanalyzed by XRPD (“Dry XRPD”). Finally, the XRPD plate was stored at 40° C./75% RH for 24-48 hour and then samples were reanalyzed (“40° C./75% RH XRPD”). All the damp and dried samples analyzed were found to be amorphous by XRPD. After a series of temperature cycling, anti-solvent addition, and/or evaporation, the isolated materials were observed to be solids from all the solvent systems assessed, except 2-propanol: water (75:25% v/v) from which oil was obtained.

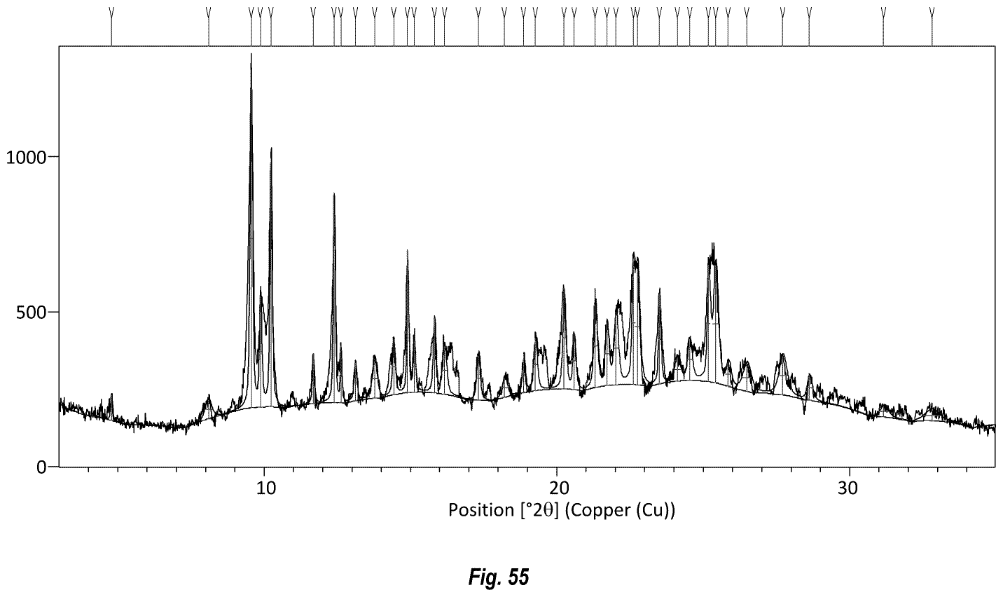

After 24-48 hours of storage at 40° C./75% RH, the samples obtained from MEK and 2-propanol: water (75:25% v/v) were found to have converted from amorphous material to a crystalline or poorly crystalline Compound 1 mono(hydrogen sulfate), which was identified as crystalline Form 10 Compound 1 mono(hydrogen sulfate). Table 49 below summarizes the forms found. An XRPD pattern for crystalline Form 10 Compound 1 mono(hydrogen sulfate) is shown in FIG. 54 with the peaks picked in FIG. 55. A corresponding peak table to FIG. 55 is found at Table 18.

An XRPD pattern of amorphous Compound 1 mono(hydrogen sulfate) can be found at FIG. 56.

TABLE 49
Salt screen - Sulfuric Acid, 1 equiv.

| | | XRPD Analysis | | |
|---|---|---|---|---|
| Solvent System | AS | Damp | Dry | 40° C. 75% RH |
| Acetonitrile | tBME | A | A | A |
| Ethanol | Heptane | A | A | A |
| Ethyl acetate | Heptane | A | A | A |
| MEK | Heptane | A | A | 10 |
| THF | Heptane | A | A | A |
| IPA:H₂O (75:25% v/v) | tBME | A | A | 10 |

A = amorphous
10 = Crystalline Form 10 mono(hydrogen sulfate)

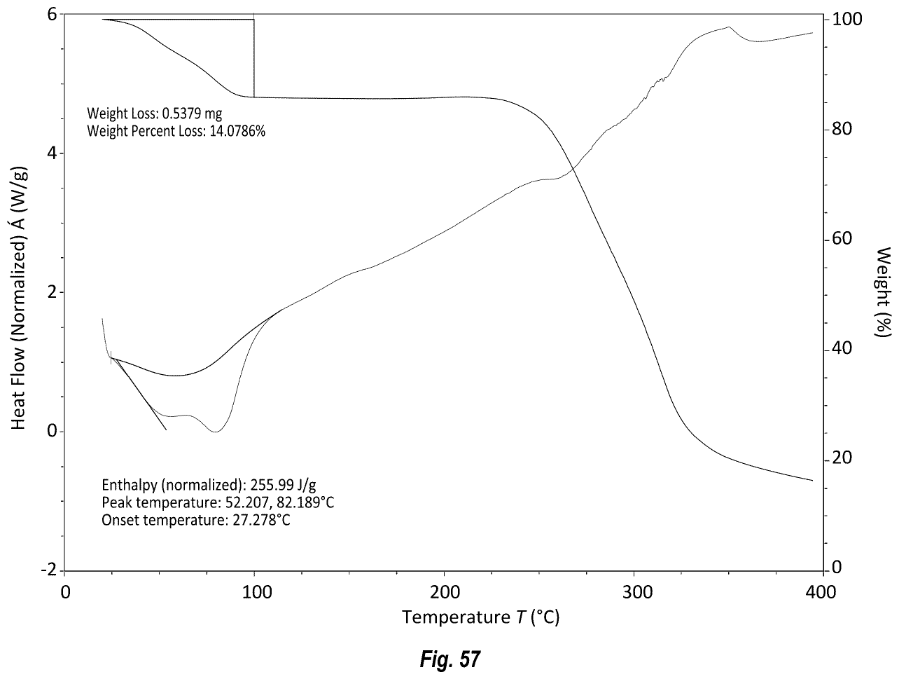

Crystalline Form 10 Compound 1 mono(hydrogen sulfate) obtained from MEK was further analyzed by TG/DSC. Mass loss of 14.1% was observed from the outset to 100° C. (this corresponds to 5.7 equiv. of water) as shown in FIG. 57. Broad overlapping endothermic events were observed from peak onset 27° C. (peaks at 52° C. and 82° C.).

Example 14-Crystalline Form 4 Compound 1 mono(hydrogen sulfate) and Crystalline Form 8 Compound 1 mono(hydrogen sulfate): Into a 20 mL vial containing about 500 mg of crystalline Form 2 Compound 1 di(hydrogen sulfate) trihydrate, 4 mL of water was added to dissolve the solids at ca. 40° C. The resulting clear solution was then stirred at 5° C. for 2-4 days. Solids were observed to be present in the solution after >24 h of stirring. The solids were isolated via vacuum filtration and analyzed by XRPD (damp). The solid samples were then air-dried under ambient conditions for 24 hr or dried under reduced pressure.

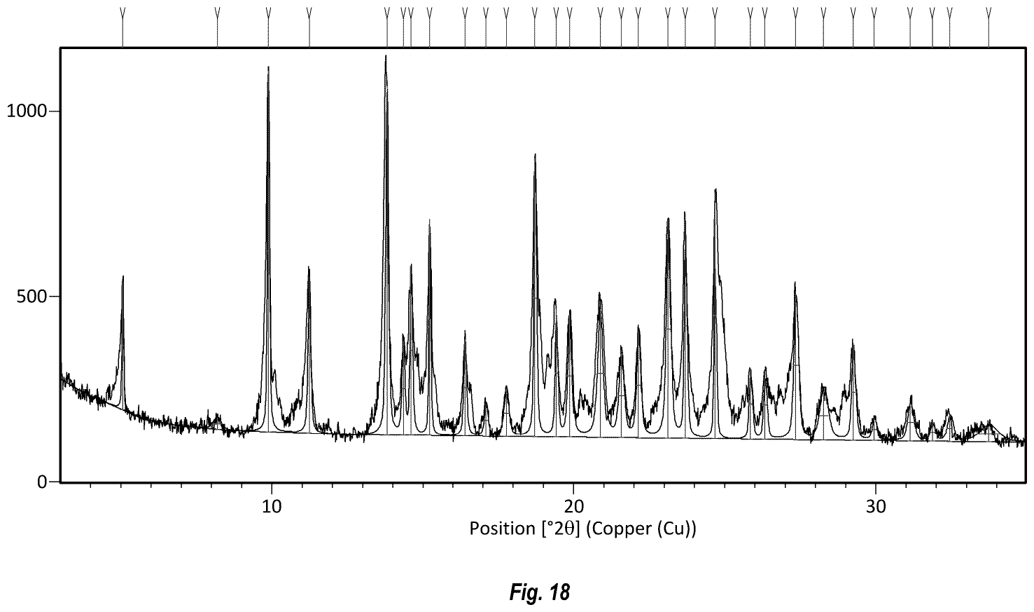

XRPD results of the damp and air-dried samples were consistent with crystalline Form 4 Compound 1 mono(hydrogen sulfate); some peak shifts/differences were observed in the dried sample as seen in FIGS. 58 and 60. The sample dried under reduced pressure showed a poorly crystalline diffractogram with other peaks (corresponding to crystalline Form 8 Compound 1 mono(hydrogen sulfate)) (FIG. 59). Additional exemplary XRPD patterns for crystalline Form 8 Compound 1 mono(hydrogen sulfate) are shown in FIGS. 17 and 18.

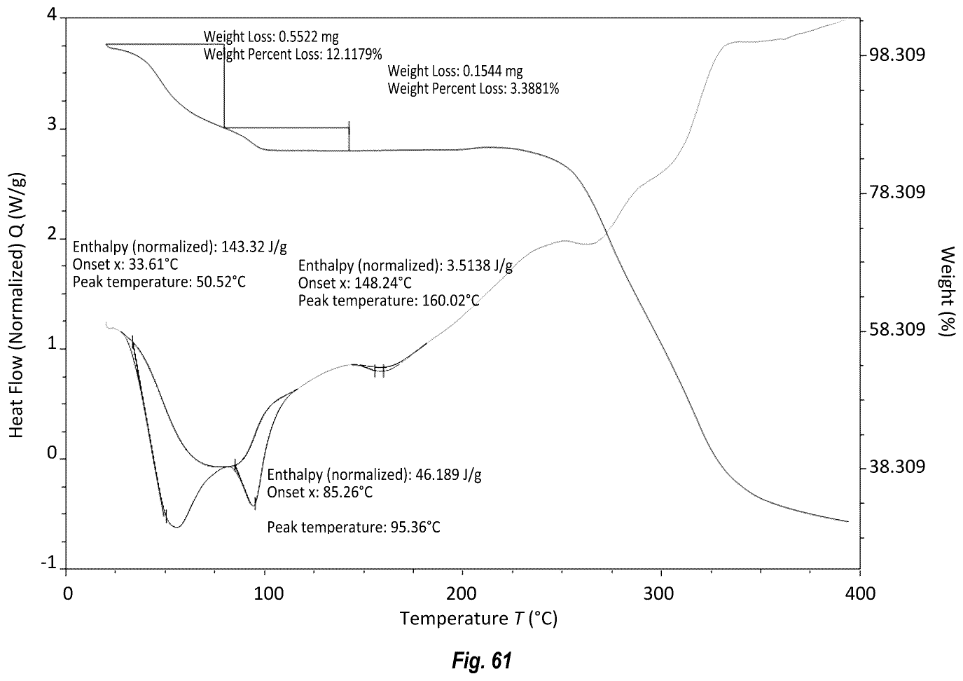

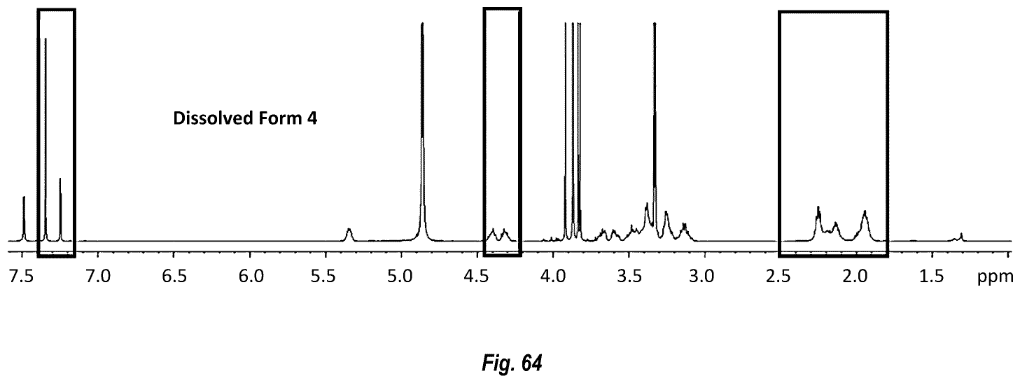

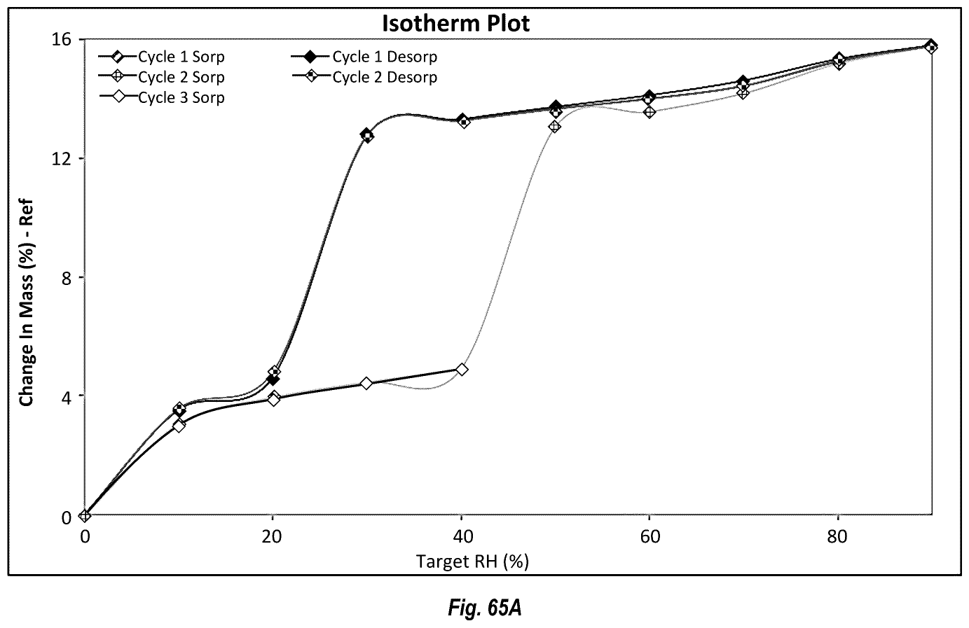

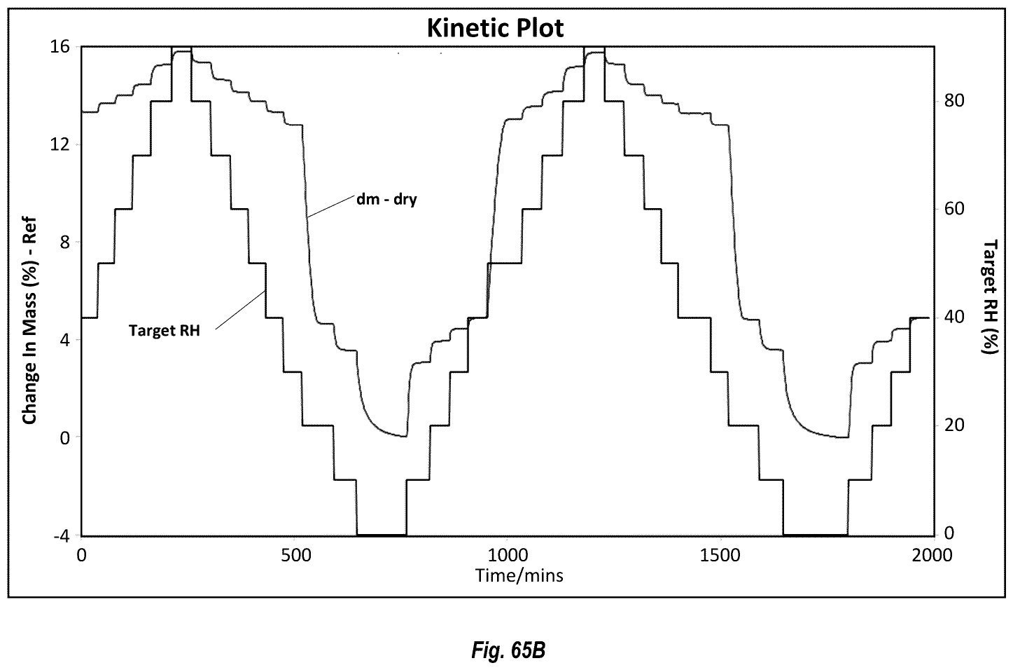

The air-dried sample (crystalline Form 4 Compound 1 mono(hydrogen sulfate)) was further characterized by TG/DSC (FIG. 61), DSC (FIG. 62), KF, FT-IR (FIG. 63), ¹H-NMR (FIG. 64), DVS (FIGS. 65A and 65B), HPLC (solid purity), and VT-XRPD. HPLC-CAD was done to obtain a general idea of the quantity of a particular element within a molecular or material as it is not a precise measure. The results of the HPLC-CAD are consistent with the chemical formula of a mono(hydrogen sulfate).

TG/DSC of the air-dried sample (crystalline Form 4 Compound 1 mono(hydrogen sulfate)) showed mass losses of 12.1% and 3.4% between 20° C.-140° C. (FIG. 61). These corresponded with endothermic events at peak onsets 34° C. and 85° C. (peaks at 50° C. and 95° C.). These are likely due to water loss. A shallow endothermic event was observed at peak onset 148° C. (peak at 160° C.). This is likely due to a melting event. The thermal events observed above 200° C. are likely due to degradation.

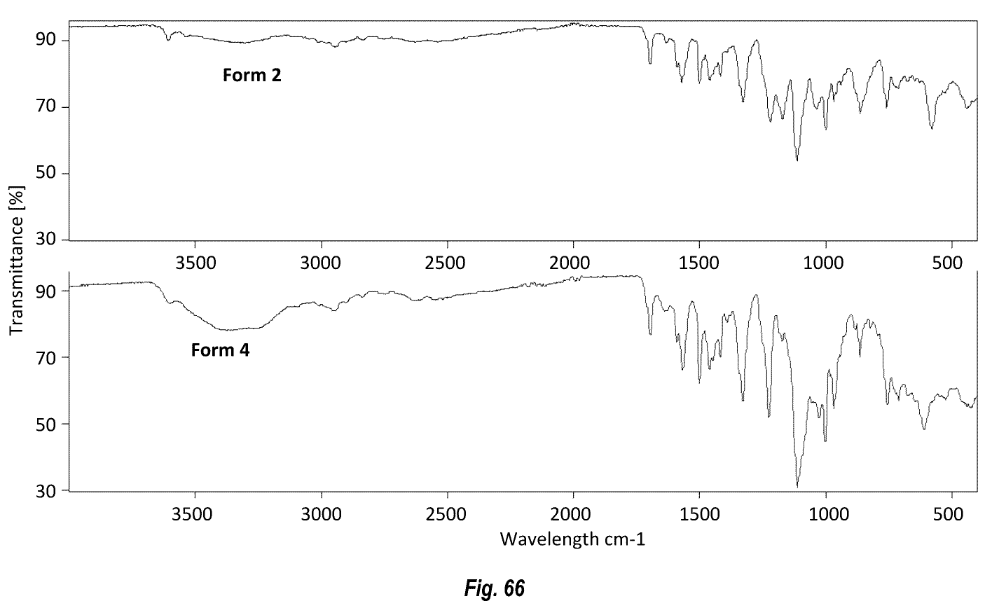

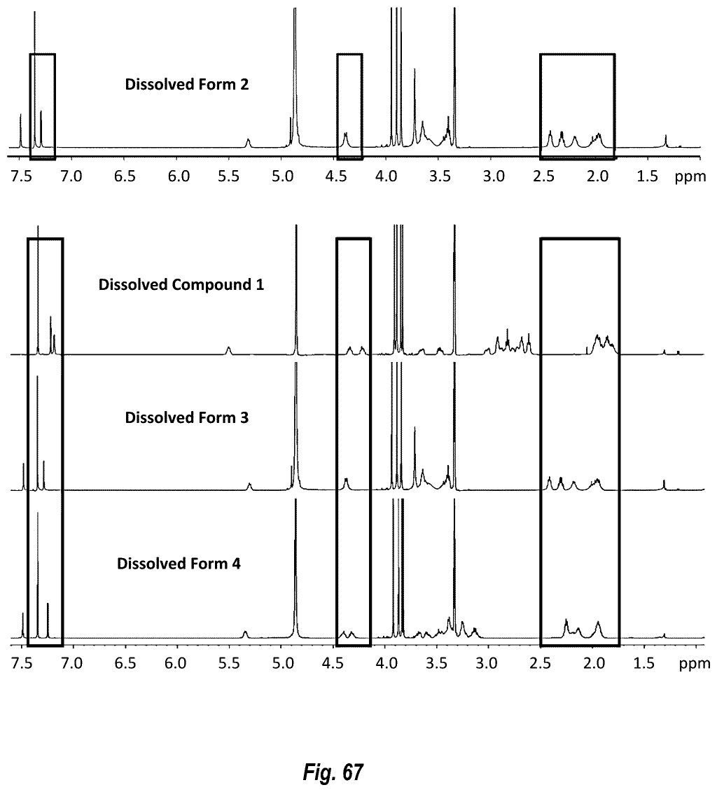

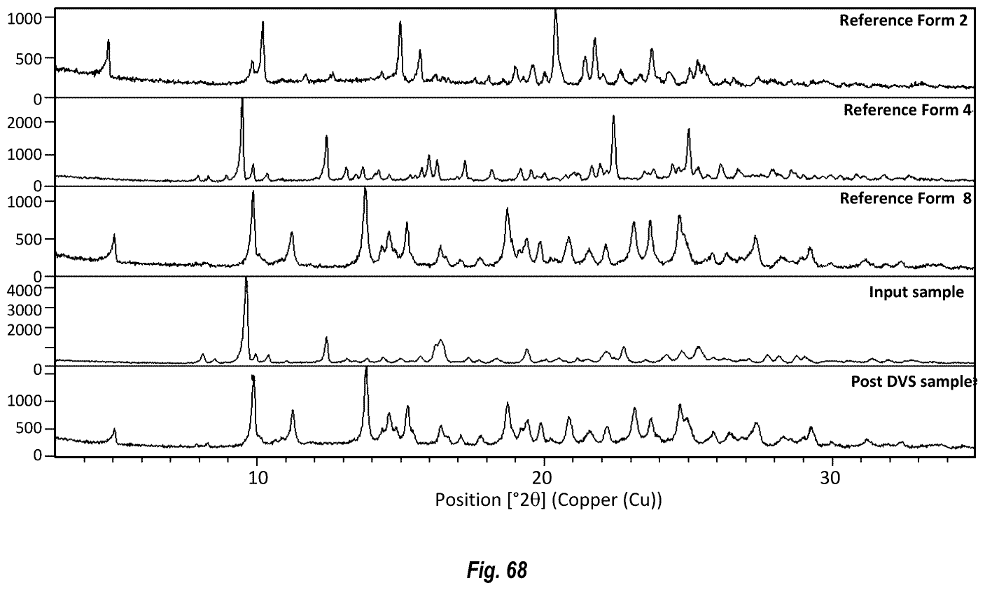

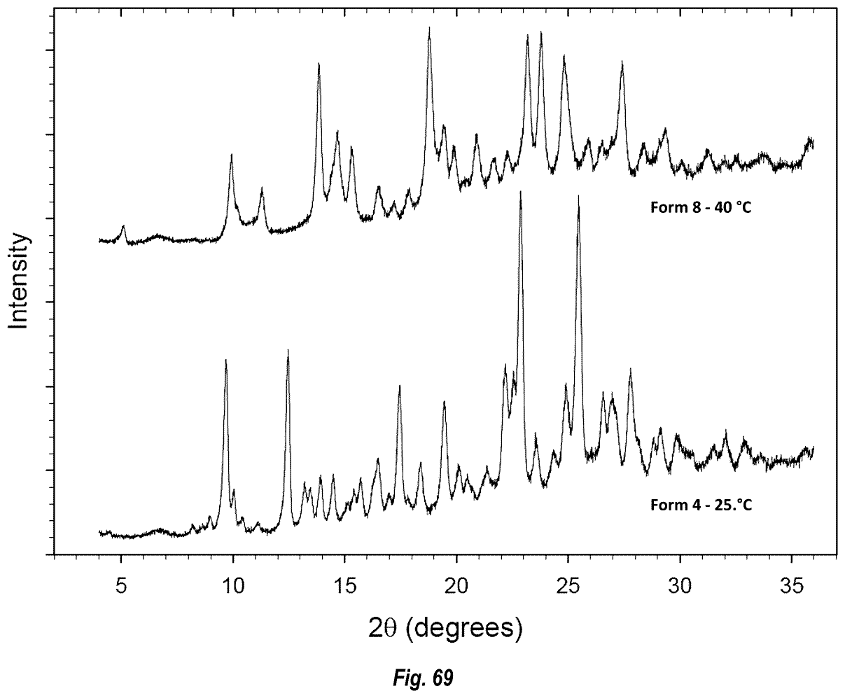

DSC analysis showed an endothermic event at peak onset 69° C. (peak at 82° C.). This is likely water loss (FIG. 62). A shallow endothermic event was observed at peak onset 113° C. (peak at 119° C.). KF analysis of the air-dried sample (crystalline Form 4 Compound 1 mono(hydrogen sulfate)) indicated an average moisture content of 14.8% w/w. This corresponds to 7.1 mole equiv. of water. FT-IR spectrum was similar to that of crystalline Form 2 Compound 1 di(hydrogen sulfate) trihydrate, but with slight differences (see FIG. 66). A ¹H-NMR spectrum showed some peak shifts that were not observed in Compound 1 (free base), crystalline Form 2 Compound 1 di(hydrogen sulfate) trihydrate and crystalline Form 3 Compound 1 di(hydrogen sulfate) as seen in the overlay spectra at FIG. 67. DVS analyses in FIGS. 65A and 65B showed a moisture uptake of ca. 4.9% between 40-40% RH during the sorption cycles. A steep uptake of 8.1% was observed between 40-50% RH. This might be due to possible hydration/form change. A further gradual uptake of ca. 2.2% was observed between 50-80% RH. A steep loss of ca. 8.0% was observed between 20-30% RH. This might be due to possible dehydration/solid form change. XRPD analysis of the post-DVS sample showed conversion to crystalline Form 8 Compound 1 mono(hydrogen sulfate) in FIG. 68. Crystalline Form 4 Compound 1 mono(hydrogen sulfate) is likely a hydrated form given that there are about 7 mole equivalents of water.

HPLC analysis of the air-dried sample (crystalline Form 4 Compound 1 mono(hydrogen sulfate)) indicated a solid purity of 99.66% area. HPLC-CAD indicated the presence of 0.85 mole equiv. of sulfate which evidences a mono(hydrogen sulfate). VT-XRPD analysis carried out between 25-110° C. showed conversion to crystalline Form 8 Compound 1 mono(hydrogen sulfate) between 40-70° C. as seen in FIG. 69. Upon heating to greater than 90° C., the sample turned amorphous as determined by XRPD. Table 50 below summarizes the experiments and results.

TABLE 50
Results of VT-XRPD analysis for Crystalline
Form 4 Compound 1 mono(hydrogen sulfate)

| Cycle | Temp (° C.) | Hold time (min) | XRPD Results |
|---|---|---|---|
| Heating | 25 | 0 | M4 |
| | 40 | 60 | M8 |
| | 50 | 60 | M8 |
| | 70 | 60 | M8 |
| Cooling | 30 | 10 | M8 |
| Heating | 50 | 60 | M8 |
| | 70 | 60 | M8 |
| | 90 | 60 | Amorphous |
| | 100 | 60 | Amorphous |
| | 110 | 60 | Amorphous |

M4 = Crystalline Form 4 mono(hydrogen sulfate)
M8 = Crystalline Form 8 mono(hydrogen sulfate)
Amorphous = Amorphous mono(hydrogen sulfate)

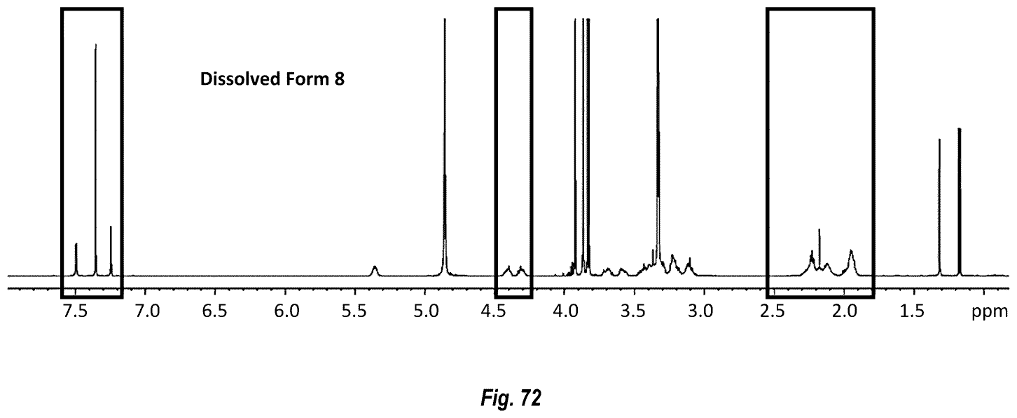

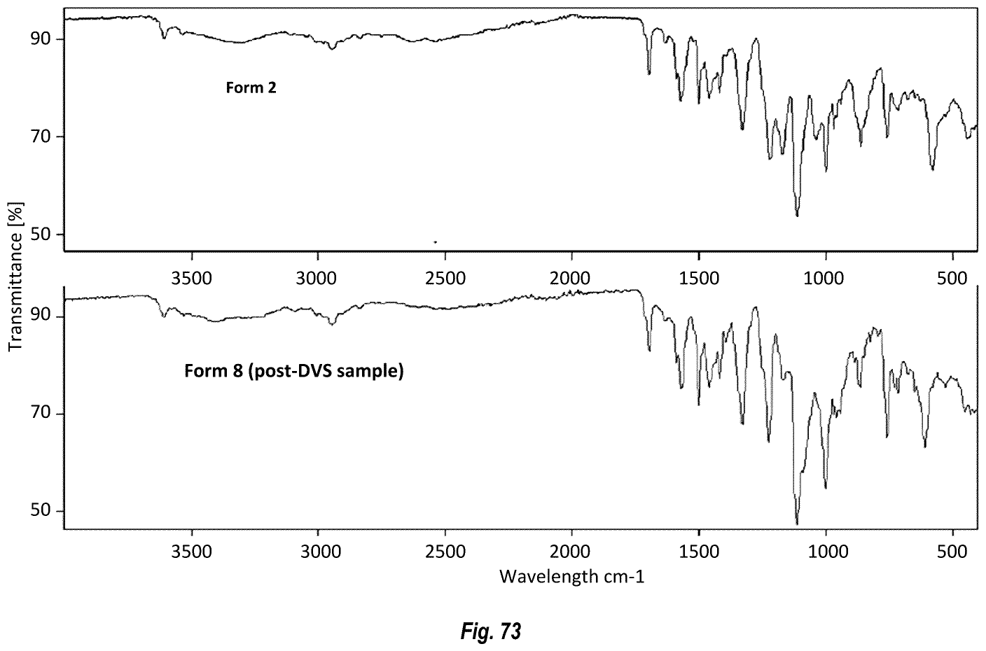

Crystalline Form 8 Compound 1 mono(hydrogen sulfate) was further characterized using TG/DSC (FIG. 70), FT-IR (FIG. 71), ¹H-NMR (FIG. 72), and HPLC-CAD. TG/DSC analysis on crystalline Form 8 Compound 1 mono(hydrogen sulfate) that was generated after DVS analysis of crystalline Form 4 Compound 1 mono(hydrogen sulfate) showed mass loss of 7.2% between 20° C.-135° C. This corresponded with an endothermic event at peak onset 42° C. (peak at 85° C.). This is likely due to water loss (7.2%=3.14 mole equiv. of H₂O theoretically). An endothermic event was observed at peak onset 144° C. (peak at 158° C.). This is likely due to a melting event. The thermal events observed above 200° C. are likely due to degradation. The FT-IR spectrum was similar to that of crystalline Form 2 Compound 1 di(hydrogen sulfate) trihydrate, but with slight differences as seen in the overlay spectrum of FIG. 73. HPLC-CAD indicated the presence of 0.84 mole equiv. of sulfate.

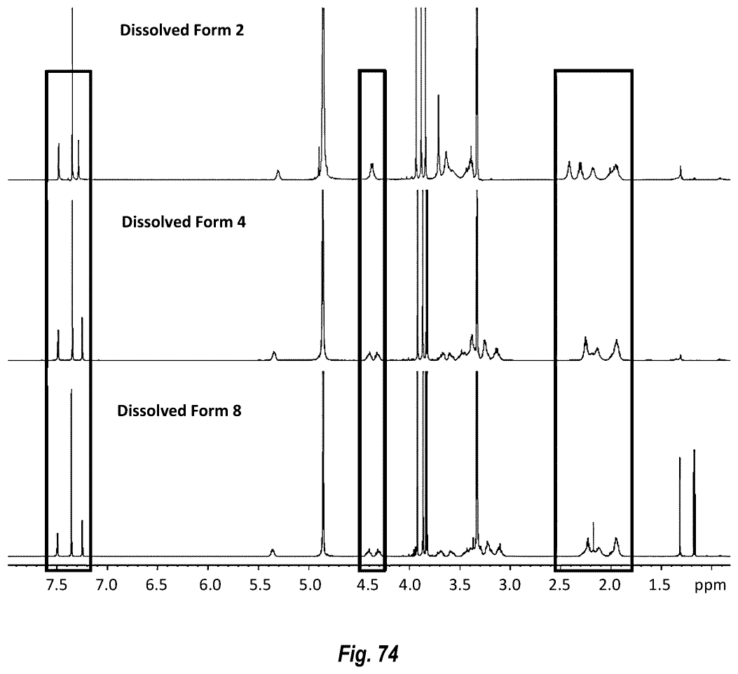

¹H-NMR spectrum on the crystalline Form 8 Compound 1 mono(hydrogen sulfate) that formed after drying crystalline Form 4 Compound 1 mono(hydrogen sulfate) under vacuum at ambient temperature showed some peak shifts compared with Compound 1 (free base) and crystalline Form 2 Compound 1 di(hydrogen sulfate) trihydrate. The shifts were consistent with crystalline Form 4 Compound 1 mono(hydrogen sulfate) as shown in FIG. 74.

The data obtained suggested that crystalline Form 8 Compound 1 mono(hydrogen sulfate) is likely to be a lower hydrated form (about 3 mole equiv. water) of the mono(hydrogen sulfate) of Compound 1 compared with crystalline Form 4 Compound 1 mono(hydrogen sulfate).

Example 15-Stabilities Studies-Crystalline Form 3 Compound 1 di(hydrogen sulfate) and Crystalline Form 4 Compound 1 mono(hydrogen sulfate): Seven-day stability studies were carried out on the prepared crystalline Form 3 Compound 1 di(hydrogen sulfate) and crystalline Form 4 Compound 1 mono(hydrogen sulfate) using the following conditions: (1) Ambient light, temperature and humidity conditions (open vial); (2) 40° C./75% RH (open vial); and (3) 80° C. (closed vial). After 7 days of storage under the specified conditions, XRPD and HPLC analyses were carried out on the residual solid materials, and the results are summarized in Table 51.

TABLE 51
Results of Stability Studies

| Input Material | Conditions | XRPD | HPLC (% area) |
|---|---|---|---|
| Form 3 | Input sample | 3 | 99.83 |
| di(hydrogen | Ambient | 6 | 99.84 |
| sulfate) | 40° C./75% RH | 2 | 99.82 |
| | 80° C. | 3 + 9 | 99.80 |
| Form 4 | Input sample | M4 | 99.66 |
| mono(hydrogen | Ambient | M4 | 99.89 |
| sulfate) | 40° C./75% RH | M4 | 99.87 |
| | 80° C. | Amorphous* | 99.64 |

2 = Crystalline Form 2 di(hydrogen sulfate) trihydrate
3 = Crystalline Form 3 di(hydrogen sulfate)
M4 = Crystalline Form 4 mono(hydrogen sulfate)
6 = Crystalline Form 6 di(hydrogen sulfate)
3 + 9 = Mixture of Crystalline Form 3 di(hydrogen sulfate) and Crystalline Form 9 di(hydrogen sulfate)
*Predominantly

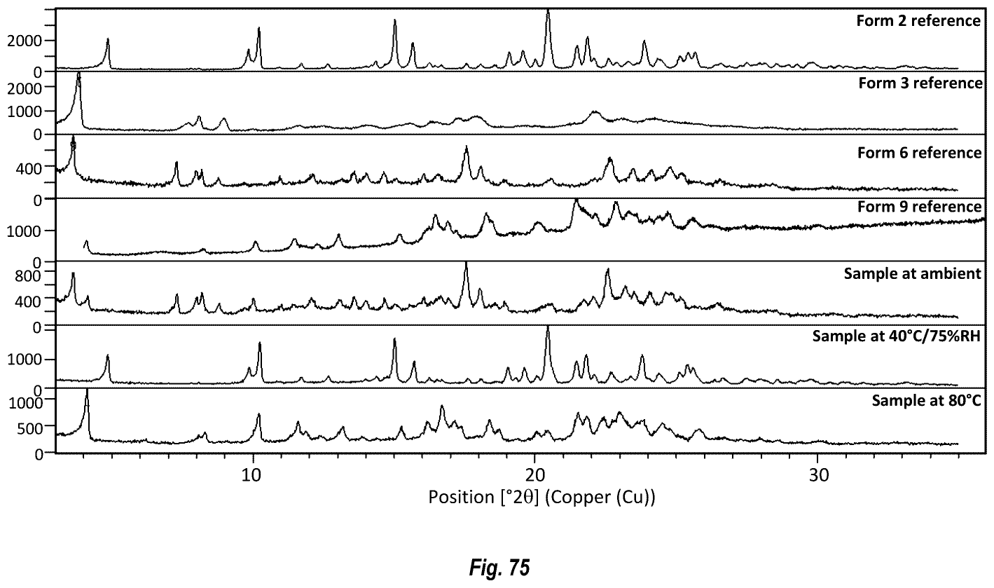

For the crystalline Form 3 Compound 1 di(hydrogen sulfate) input, conversion to crystalline Form 6 Compound 1 di(hydrogen sulfate) was observed at ambient conditions. Conversion to crystalline Form 2 Compound 1 di(hydrogen sulfate) trihydrate was observed at 40° C./75% RH. A mixture of crystalline Form 3 Compound 1 di(hydrogen sulfate) and crystalline Form 9 Compound 1 di(hydrogen sulfate) was observed at 80° C. The purity of the samples was similar to the input material for all the conditions assessed. FIG. 75 shows the XRPD patterns associated with the crystalline Form 3 Compound 1 di(hydrogen sulfate) input.

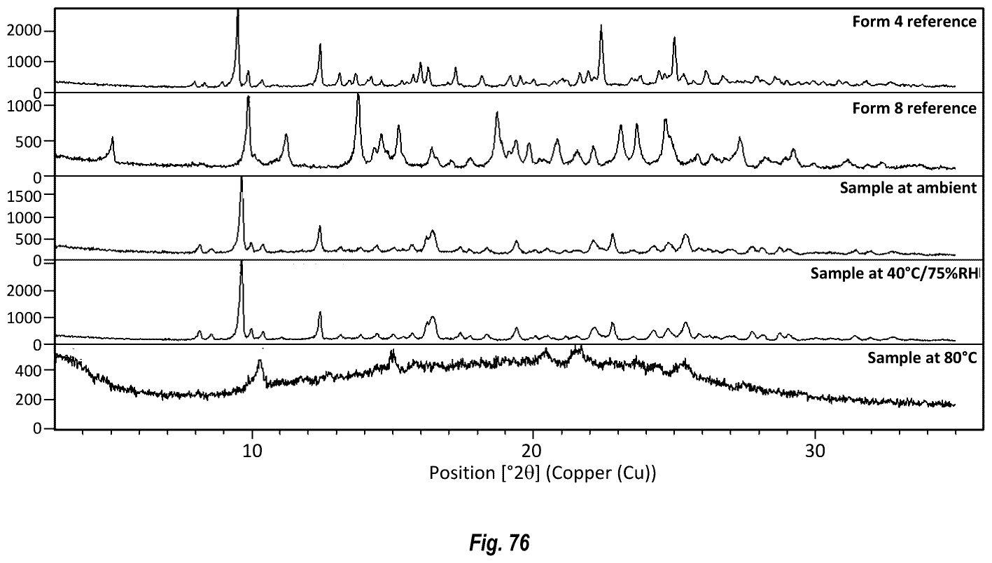

For the crystalline Form 4 Compound 1 mono(hydrogen sulfate) input, crystalline Form 4 Compound 1 mono(hydrogen sulfate) was retained at ambient conditions and at 40° C./75% RH. The sample stored at 80° C. was found to be predominantly amorphous. The purity of the samples was similar to the input material for all the conditions assessed. FIG. 76 shows the XRPD patterns associated with the crystalline Form 4 Compound 1 mono(hydrogen sulfate) input.

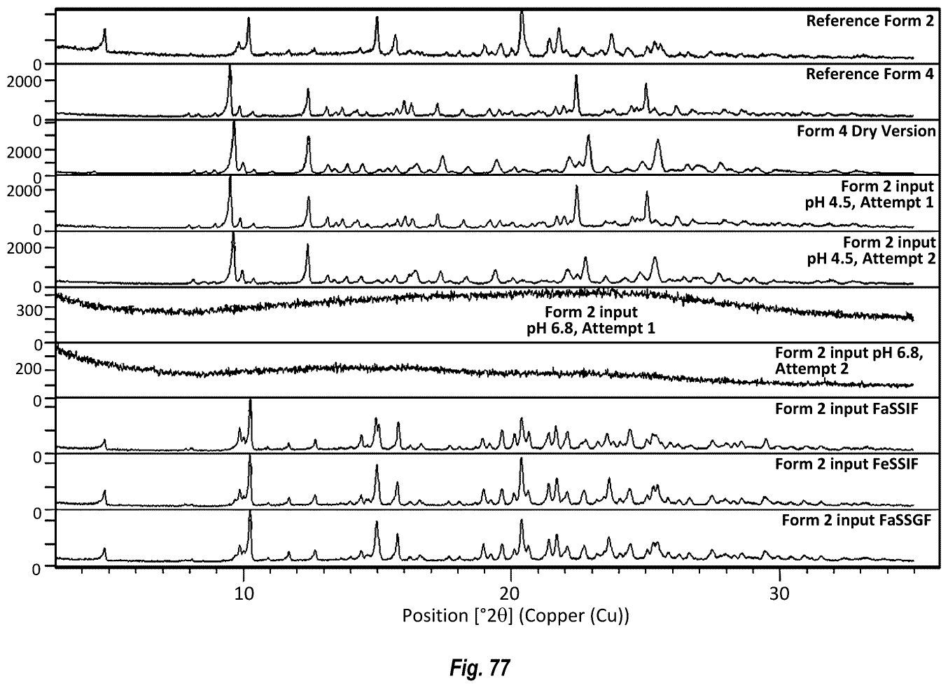

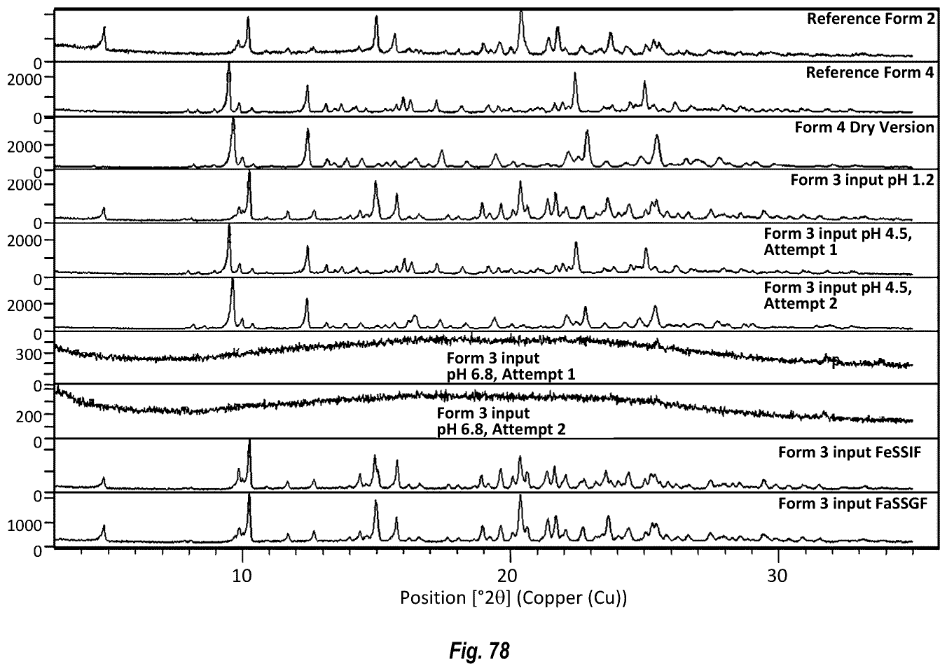

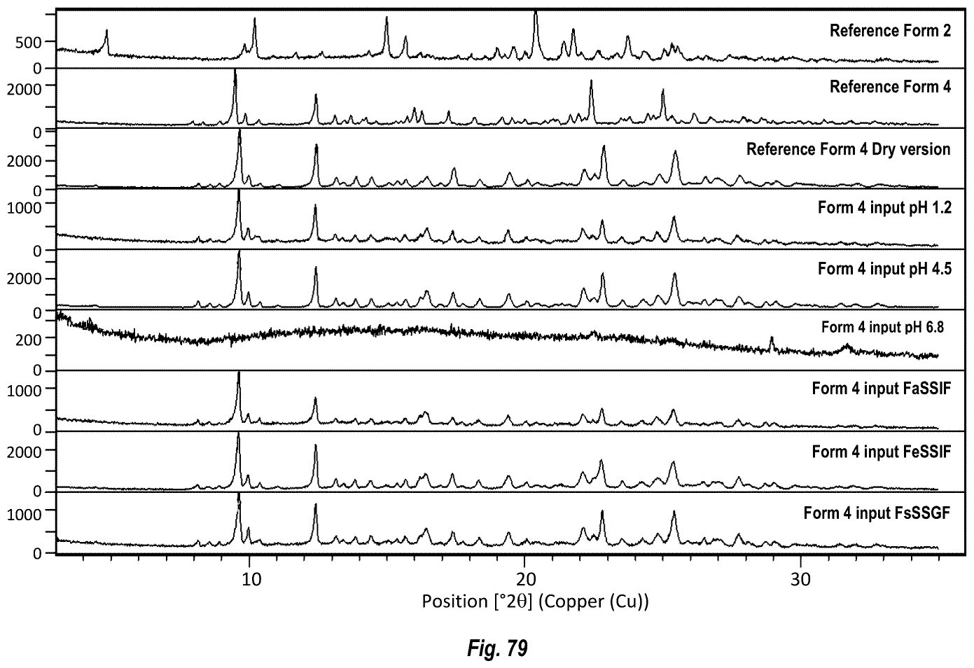

Example 16—pH Solubility Studies—(1) Crystalline Form 2 Compound 1 di(hydrogen sulfate) trihydrate, (2) Crystalline Form 3 Compound 1 di(hydrogen sulfate), and (3) Crystalline Form 4 Compound 1 mono(hydrogen sulfate): pH solubility studies were carried out on the prepared crystalline Form 2 Compound 1 di(hydrogen sulfate) trihydrate, crystalline Form 3 Compound 1 di(hydrogen sulfate) and crystalline Form 4 Compound 1 mono(hydrogen sulfate) in the following media: (1) HCl/KCl, pH 1.2; (2) Acetate, pH 4.5; (3) Phosphate, pH 6.8; (4) FaSSIF; (5) FeSSIF; and (6) FaSSGF. Slurries of the hydrogen sulfate forms were prepared in the appropriate medium and stirred at 25° C. For the experiments in the buffer media, the pH of the slurry samples was measured and adjusted as required after 1 hour and 23 hours of the slurrying. Additional solid was added where dissolution was observed post-adjustment. The pH of the samples in the biorelevant media was measured at the 23 hour timepoint. No pH adjustment was made for the biorelevant media. After a total of 24 hours of slurrying, the mother liquors were extracted via centrifugation, and the concentrations and solution purity were determined by HPLC. The residual solids were isolated and analyzed by XRPD and are shown in FIG. 77, FIG. 78, and FIG. 79. The assessment was repeated for some of the samples, where required.

The results for the crystalline Form 2 Compound 1 di(hydrogen sulfate) trihydrate are summarized in Table 52 and the XRPD patterns are in FIG. 77. A solubility of >75 mg/mL was observed in the pH 1.2 and 4.5 buffers and in all the biorelevant media. A solubility of <20 mg/mL was observed in pH 4.5 buffer media from Attempt 1. The solution purity of the extracted mother liquors was >99% for all samples except pH 4.5 Attempt 1, which was 89% area. This chromatogram showed an early eluting impurity of ca. 9.27% at RRT 0.28. XRPD of the residual solids showed crystalline Form 4 Compound 1 mono(hydrogen sulfate) in the pH 4.5 media; the Attempt 2 sample was consistent with the air-dried crystalline Form 4 Compound 1 mono(hydrogen sulfate) sample where some peak shifts/changes were observed. Amorphous material was obtained in the pH 6.8 media and crystalline Form 2 Compound 1 di(hydrogen sulfate) trihydrate was obtained from the biorelevant media. No solids were obtained from the pH 1.2 media.

TABLE 52
Results for the Crystalline Form 2 Compound 1 di(hydrogen sulfate) trihydrate pH solubility studies

| | | | | | | | | ML | |
|---|---|---|---|---|---|---|---|---|---|
| Buffer | | Buffer | After 4 h | | After 24 h | | Solub. | purity | |
| Media | Attempts | addition | pH | Obs. | pH | Obs. | (mg/mL) | (% area) | XRPD |
| pH 1.2 | 1 | Slurry | 1.20 | Slurry | 0.98→ | Slurry→ | >86.7 | 99.13 | ns. |
| (HCl/KCl) | | | | | 1.22 | Clear | | | |
| pH 4.5 | 1 | Slurry | 1.18→ | Slurry | 4.53 | Slurry | 17.8 | 89.50 | M4 |
| (Acetate) | | | 4.39 | | | | | | |
| | 2 | Slurry | 1.16→ | Slurry→ | 1.68→ | Slurry | 94.5 | 99.76 | M4 |
| | | | 4.45 | Clear | 4.62 | | | | |
| pH 6.8 | 1 | Slurry | 1.16→ | Slurry→ | 1.01→ | Slurry→ | 12.0 | 99.43 | A |
| (Phosphate) | | | 6.72 | Clear | 6.87 | Gum | | | |
| | 2 | Slurry | 1.42→ | Slurry→ | 4.79→ | Slurry→ | 6.1 | n/a | A |
| | | | 6.92 | Clear | 6.90 | Gum | | | |
| FaSSIF | 1 | Slurry | — | — | 1.12 | Slurry | 76.6 | 99.80 | 2 |
| FeSSIF | 1 | Slurry | — | — | 1.07 | Slurry | 133.3 | 99.72 | 2 |
| FaSSGF | 1 | Slurry | — | — | 0.93 | Slurry | 81.1 | 99.67 | 2 |

M4 = Crystalline Form 4 mono(hydrogen sulfate)
2 = Crystalline Form 2 di(hydrogen sulfate) trihydrate
A = Amorphous
n/a = not applicable

The results for the crystalline Form 3 Compound 1 di(hydrogen sulfate) are summarized in Table 53 and the XRPD patterns are in FIG. 78. A solubility of >50 mg/mL was observed in all the media assessed, except in the pH 4.5 media where a solubility of 20 mg/mL was observed in Attempt 2. The solution purity of the extracted mother liquors was >95% area. XRPD of the residual solids showed crystalline Form 4 Compound 1 mono(hydrogen sulfate) in the pH 4.5 media; the Attempt 2 sample was consistent with the air-dried crystalline Form 4 Compound 1 mono(hydrogen sulfate) sample where some peak shifts/changes were observed. Amorphous material was obtained in the pH 6.8 media. Crystalline Form 2 Compound 1 di(hydrogen sulfate) trihydrate was obtained from the pH 1.2 buffer and the biorelevant media.

TABLE 53
Results for the Crystalline Form 3 Compound 1 di(hydrogen sulfate) pH solubility studies

| | | | | | | | | ML | |
|---|---|---|---|---|---|---|---|---|---|
| Buffer | | Buffer | After 4 h | | After 24 h | | Solub. | purity | |
| Media | Attempts | addition | pH | Obs. | pH | Obs. | (mg/mL) | (% area) | XRPD |
| pH 1.2 | 1 | Clear→ | 1.08 | Slurry | 1.13 | Slurry | 75.7 | 99.56 | 2 |
| (HCl/KCl) | | Slurry | | | | | | | |
| pH 4.5 | 1 | Clear→ | 1.16→ | Slurry | 4.76→ | Slurry | 54.2 | 99.59 | M4 |
| (Acetate) | | Slurry | 4.60 | | 4.58 | | | | |
| | 2 | Clear→ | 1.61→ | Slurry | 4.88→ | Slurry | 20.0 | 95.53 | M4 |
| | | Slurry | 4.65 | | 4.56 | | | | |
| pH 6.8 | 1 | Clear→ | 1.01→ | Slurry→ | 5.57→ | Clear→ | 122.5 | 99.87 | A |
| (Phosphate) | | Slurry | 6.79 | Clear | 6.70 | Gum | | | |
| | 2 | Clear→ | 1.67→ | Slurry→ | 1.57→ | Slurry→ | 152.8 | 99.16 | A |
| | | Slurry | 6.95 | Clear | 6.71 | Gum | | | |
| FaSSIF | 1 | Clear→ | — | — | 1.10 | Slurry | 77.1 | 99.76 | 2 |
| | | Slurry | | | | | | | |
| FeSSIF | 1 | Cloudy | — | — | 1.01 | Cloudy | ≥208.3 | 99.90 | ns. |
| FaSSGF | 1 | Clear→ | — | — | 0.87 | Slurry | 72.0 | 99.51 | 2 |
| | | Slurry | | | | | | | |

2 = Crystalline Form 2 di(hydrogen sulfate) trihydrate
M4 = Crystalline Form 4 mono(hydrogen sulfate)
A = amorphous
ns. = not enough solid

The results for the crystalline Form 4 Compound 1 mono(hydrogen sulfate) are summarized in Table 54 and the XRPD patterns are in FIG. 79. A solubility of >100 mg/mL was observed in the pH 1.2 and FeSSIF media. A solubility of 30-80 mg/mL was obtained in the other media assessed. The solution purity of the extracted mother liquors was >98% area. The XRPD of the residual solids showed crystalline Form 4 Compound 1 mono(hydrogen sulfate) from all the media assessed, except from the pH 6.8 media where amorphous material was obtained. The crystalline Form 4 Compound 1 mono(hydrogen sulfate) diffractograms were consistent with the air-dried crystalline Form 4 Compound 1 mono(hydrogen sulfate) sample where peak shifts/changes were observed.

TABLE 54
Results for the Crystalline Form 4 Compound 1 mono(hydrogen sulfate) pH solubility studies

| Buffer | Buffer | After 4 h | | After 24 h | | Solub. | ML purity | |
|---|---|---|---|---|---|---|---|---|
| Media | addition | pH | Obs. | pH | Obs. | (mg/mL) | (% area) | XRPD |
| pH 1.2 | Slurry | 1.60→ | Slurry→ | 1.57→ | Slurry | 134.1 | >99.99 | M4 |
| (HCl/KCl) | | 1.33 | Clear | 1.33 | | | | |
| pH 4.5 | Slurry | 4.23→ | Slurry | 4.43 | Slurry | 46.9 | 99.86 | M4 |
| (Acetate) | | 4.42 | | | | | | |
| pH 6.8 | Slurry | 4.69→ | Slurry | 5.19→ | Slurry→ | 62.0 | 98.30 | A |
| (Phosphate) | | 6.72 | | 6.94 | Gum | | | |
| FaSSIF | Slurry | — | — | 3.93 | Slurry | 44.9 | 99.86 | M4 |
| FeSSIF | Slurry | — | — | 4.34 | Slurry | 124.2 | 99.69 | M4 |
| FaSSGF | Slurry | — | — | 1.98 | Slurry | 36.7 | 99.82 | M4 |

M4 = Crystalline Form 4 mono(hydrogen sulfate)
A = amorphous

Example 17—Hydration Mapping Studies-Crystalline Form 2 Compound 1 di(hydrogen sulfate) trihydrate, Crystalline Form 3 Compound 1 di(hydrogen sulfate), and Blend: The effects of different water activities on crystalline Form 2 Compound 1 di(hydrogen sulfate) trihydrate, crystalline Form 3 Compound 1 di(hydrogen sulfate) and a blend of the two were investigated. The “blend” was prepared by mixing crystalline Form 2 Compound 1 di(hydrogen sulfate) trihydrate and crystalline Form 3 Compound 1 di(hydrogen sulfate) in a 1:1 ratio. To the vials containing the required solid material, the appropriate solvent system was added to form slurries. The slurry samples were stirred at the required temperatures during and after the solvent addition. Where dissolution was observed post-solvent addition, additional solid was added to re-saturate the sample. After stirring at the required temperature for 48 hours, the residual solids left in the vials were analyzed by XRPD to assess the solid forms. The analyzed samples were then dried on the XRPD plate under reduced pressure at 40° C. for 24 hr. The dried samples were analyzed by XRPD. Table 55 shows the conditions considered during these studies.

TABLE 55
Conditions studied in Hydration Mapping Studies

| | Solvent Systems | | | | | | | | | |
|---|---|---|---|---|---|---|---|---|---|---|
| | Acetonitrile:Water | | | | | 2-Propanol:Water | | | | |
| Water Activity (a_{w}) | 0.1 | 0.3 | 0.5 | 0.7 | 0.9 | 0.1 | 0.3 | 0.5 | 0.7 | 0.9 |
| 1:1 Blend | ✓ | ✓ | ✓ | ✓ | ✓ | ✓ | ✓ | ✓ | ✓ | ✓ |
| Form 2 | ✓ | | | | ✓ | ✓ | | | | ✓ |
| di(hydrogen | | | | | | | | | | |
| sulfate) trihydrate | | | | | | | | | | |
| Form 3 | ✓ | | | | ✓ | ✓ | | | | ✓ |
| di(hydrogen | | | | | | | | | | |
| sulfate) | | | | | | | | | | |

| | |
|---|---|
| Temperature | 50° C., 20° C. |

The results obtained from these studies are summarized in Table 56 and Table 57 and discussed below in more detail.

TABLE 56
Results from the hydration studies - damp samples
50° C.

| Solvent Systems | Acetonitrile:Water | | | | | | | | | 2-Propanol:Water | | | | | |
|---|---|---|---|---|---|---|---|---|---|---|---|---|---|---|---|
| Water Activity (a_{w}) | 0.1 | | 0.3 | | 0.5 | | 0.7 | 0.9 | | 0.1 | 0.3 | 0.5 | 0.7 | 0.9 | |
| Blend | 1 | 3 | 1 | 3 | 3 | 2 | 2 | 2+ | M10 | 2 | 2 | 2 | 2 | 2+ | M4 |
| Form 2 di(hydrogen | 6 | | | | | | | 2 | | 2 | | | | 2 | |
| sulfate) trihydrate | | | | | | | | | | | | | | | |
| Input | | | | | | | | | | | | | | | |

| | | | | | | |
|---|---|---|---|---|---|---|
| Form 3 di(hydrogen | 3 | 6 | 2 | 1 | 6 | 2 |

sulfate) Input

20° C.

| Solvent Systems | Acetonitrile:Water | | | | | | | 2-Propanol:Water | | | | |
|---|---|---|---|---|---|---|---|---|---|---|---|---|
| Water Activity (a_{w}) | 0.1 | | 0.3 | | 0.5 | 0.7 | 0.9 | 0.1 | 0.3 | 0.5 | 0.7 | 0.9 |
| Blend | 3 | 2 | 3 | 2 | 2 | 2 | 2 | 2 | 2 | 2 | 2 | 2 |
| Form 2 di(hydrogen | 2 | | | | | | 2 | 2 | | | | 2 |
| sulfate) trihydrate | | | | | | | | | | | | |
| Input | | | | | | | | | | | | |

| | | | | | | |
|---|---|---|---|---|---|---|
| Form 3 di(hydrogen | 1 | 3 | 2 | 3 | 6 | 2 |

sulfate) Input

TABLE 57
Results from the hydration studies - dried samples
50° C.

| Solvent Systems | Acetonitrile:Water | | | | | | | | | 2-Propanol:Water | | | | |
|---|---|---|---|---|---|---|---|---|---|---|---|---|---|---|
| Water Activity (a_{w}) | 0.1 | | 0.3 | | 0.5 | | 0.7 | 0.9 | | 0.1 | 0.3 | 0.5 | 0.7 | 0.9 |
| Blend | 3 | 6 | 3 | 6 | 3 | 2 | 2 | 2 | M10 | 2 | 2 | 2 | 2 | 2+ |
| Form 2 di(hydrogen | 3+ | | | | | | | 2 | | 2 | | | | 2 |
| sulfate) trihydrate | | | | | | | | | | | | | | |
| Input | | | | | | | | | | | | | | |
| Form 3 di(hydrogen | 3, PC | | | | | | | 2 | | 1, PC | | | | 2 |
| sulfate) Input | | | | | | | | | | | | | | |

20° C.

| Solvent Systems | Acetonitrile:Water | | | | | | | 2-Propanol:Water | | | | |
|---|---|---|---|---|---|---|---|---|---|---|---|---|
| Water Activity (a_{w}) | 0.1 | | 0.3 | | 0.5 | 0.7 | 0.9 | 0.1 | 0.3 | 0.5 | 0.7 | 0.9 |
| Blend | 2 | 3 | 2 | 3 | 2 | 2 | 2 | 2 | 2 | 2 | 2 | 2 |
| Form 2 di(hydrogen | 2 | | | | | | 2 | 2 | | | | 2 |
| sulfate) trihydrate | | | | | | | | | | | | |
| Input | | | | | | | | | | | | |
| Form 3 di(hydrogen | 3, PC | | | | | | 2 | 3, PC | | | | 2 |
| sulfate) Input | | | | | | | | | | | | |

1 Crystalline Form 1 di(hydrogen sulfate)
2 Crystalline Form 2 di(hydrogen sulfate) trihydrate
3 Crystalline Form 3 di(hydrogen sulfate)
M4 Crystalline Form 4 mono(hydrogen sulfate)
6 Crystalline Form 6 di(hydrogen sulfate)
M10 Crystalline Form 10 mono(hydrogen sulfate)
PC Poorly crystalline
+ Extra peaks

Regarding the blended material (crystalline Form 2 Compound 1 di(hydrogen sulfate) trihydrate and crystalline Form 3 Compound 1 di(hydrogen sulfate)), some crystalline Form 3 Compound 1 di(hydrogen sulfate) was retained within the samples from acetonitrile:water solvent systems with a_{w}≤0.5. Crystalline Form 1 Compound 1 di(hydrogen sulfate) or crystalline Form 6 Compound 1 di(hydrogen sulfate) was also observed in the samples from the systems with a_{w}≤0.3 at 50° C. Crystalline Form 2 Compound 1 di(hydrogen sulfate) trihydrate was observed in the samples from systems with a_{w}>0.5. Crystalline Form 2 Compound 1 di(hydrogen sulfate) trihydrate was also observed in the samples from all the 2-propanol:water solvent systems. Traces of crystalline Form 4 Compound 1 mono(hydrogen sulfate) or crystalline Form 10 Compound 1 mono(hydrogen sulfate) were observed in the samples from the systems with a_{w}=0.9 at 50° C.

Regarding crystalline Form 2 Compound 1 di(hydrogen sulfate) trihydrate, conversion to crystalline Form 6 Compound 1 di(hydrogen sulfate) was observed in the sample from acetonitrile:water (a_{w}=0.1) at 50° C. This sample dried to crystalline Form 3 Compound 1 di(hydrogen sulfate). The rest of the samples were consistent with crystalline Form 2 Compound 1 di(hydrogen sulfate) trihydrate.

Regarding crystalline Form 3 Compound 1 di(hydrogen sulfate), crystalline Form 3 Compound 1 di(hydrogen sulfate) was retained in the samples from solvent systems with a_{w}=0.1. Partial conversion to crystalline Form 1 Compound 1 di(hydrogen sulfate) or crystalline Form 6 Compound 1 di(hydrogen sulfate), with some loss of crystallinity, was observed in these systems when damp. Complete conversion to crystalline Form 1 Compound 1 di(hydrogen sulfate), with some loss of crystallinity, was observed in the dried sample from 2-propanol:water a_{w}=0.1 at 50° C. Conversion to crystalline Form 2 Compound 1 di(hydrogen sulfate) trihydrate was observed in the samples from solvent systems with a_{w}=0.9.

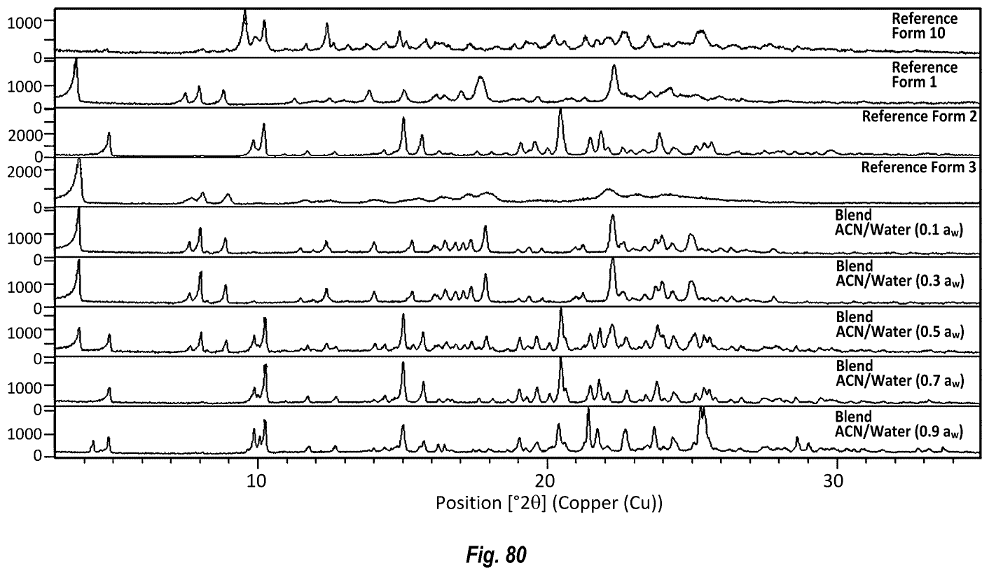

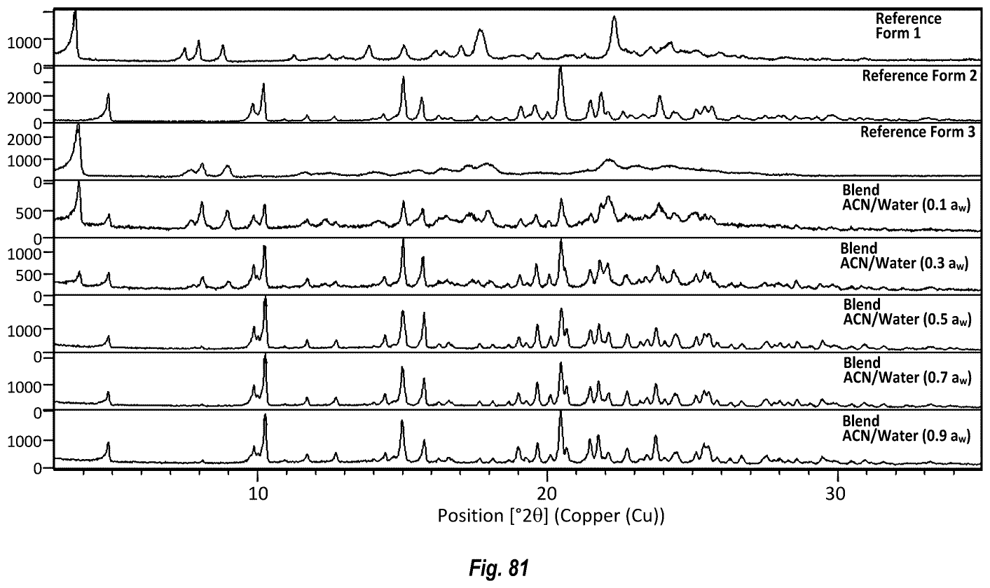

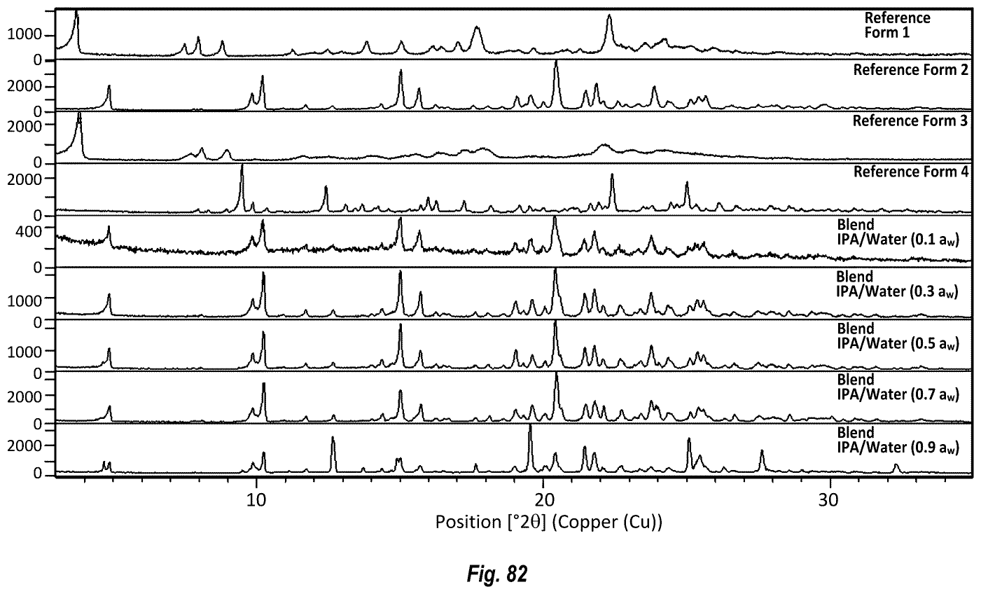

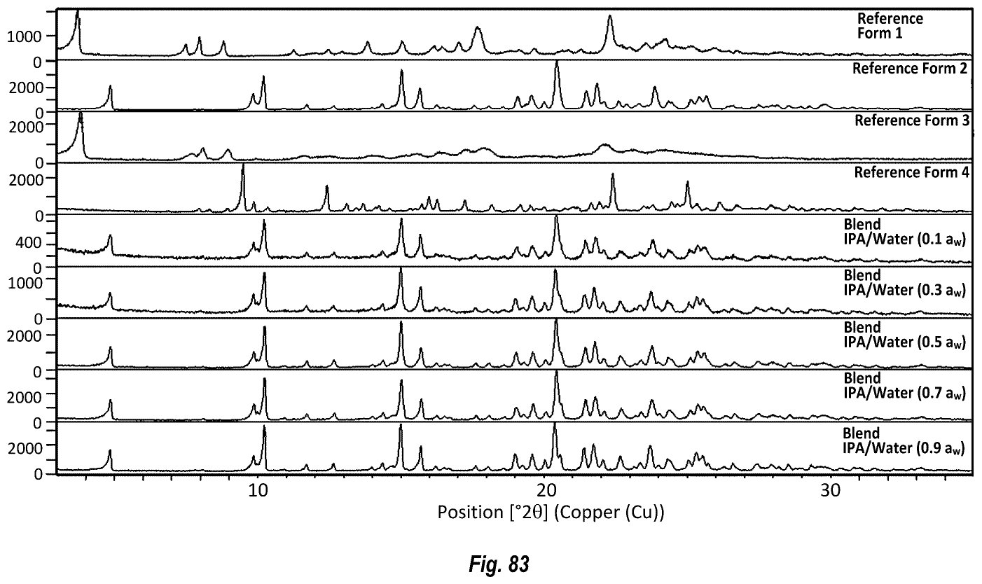

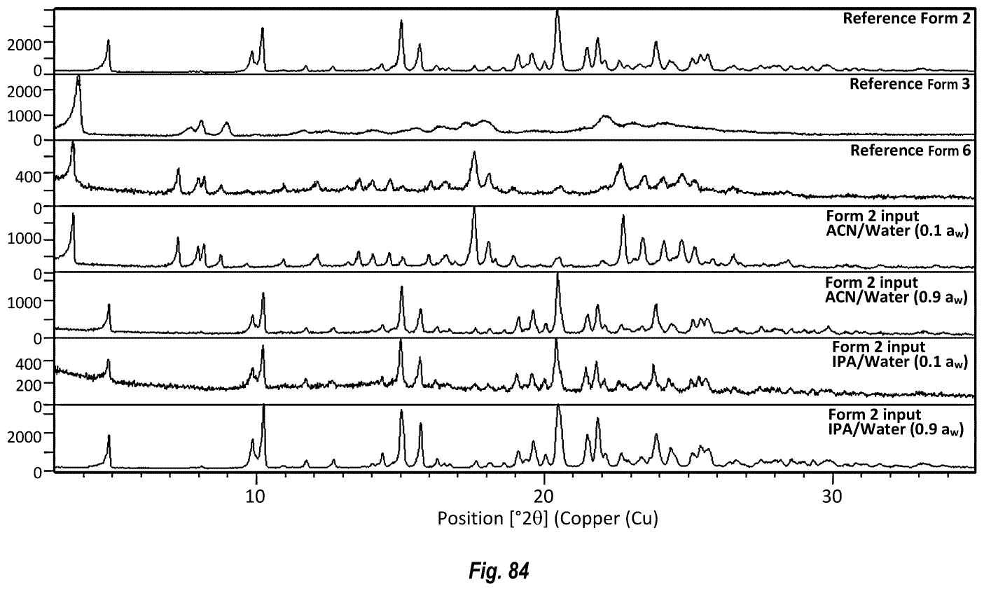

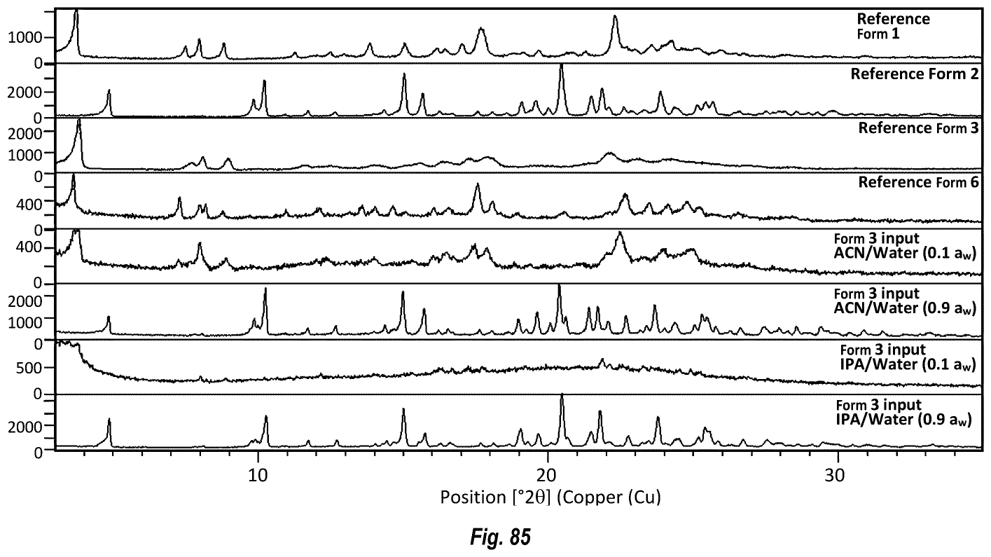

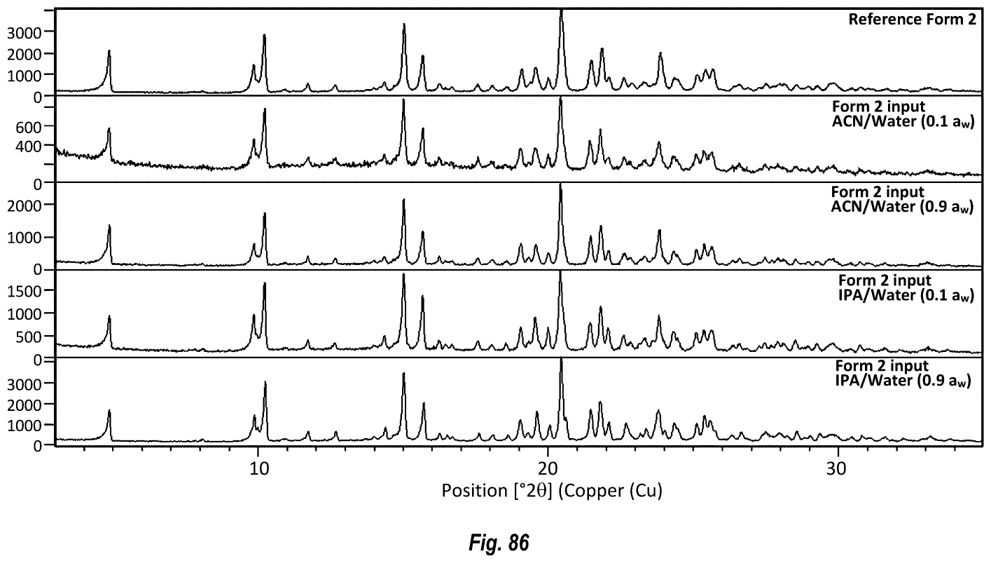

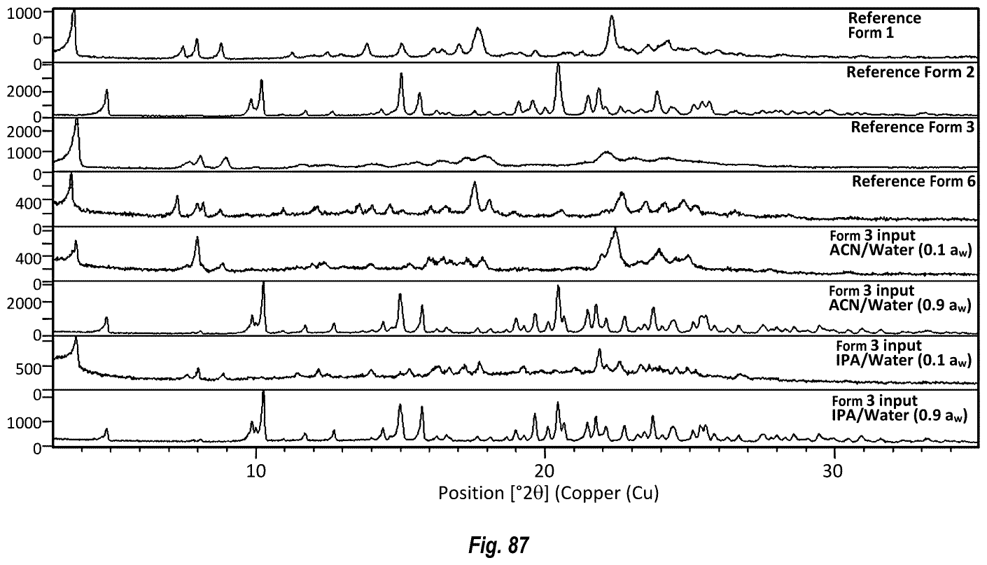

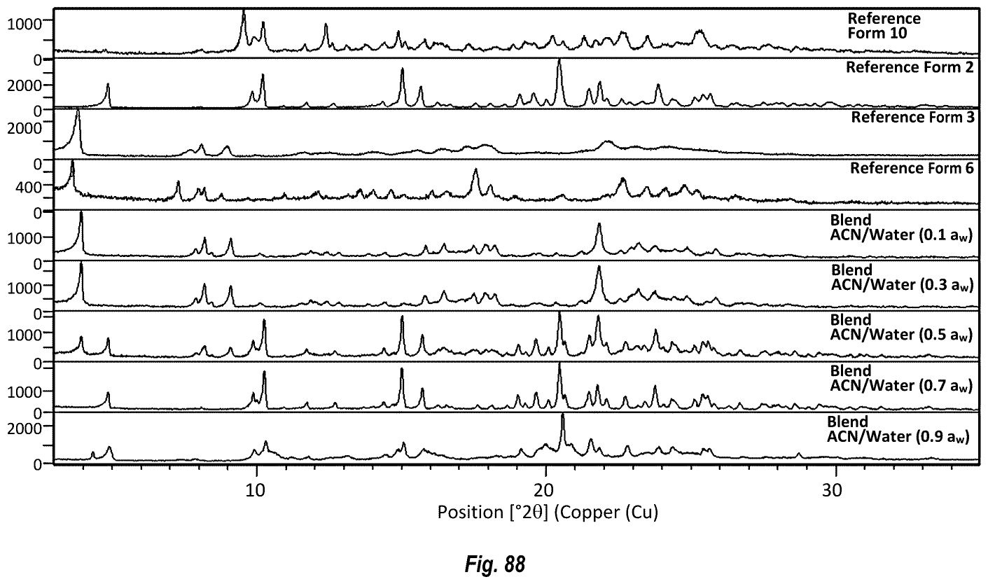

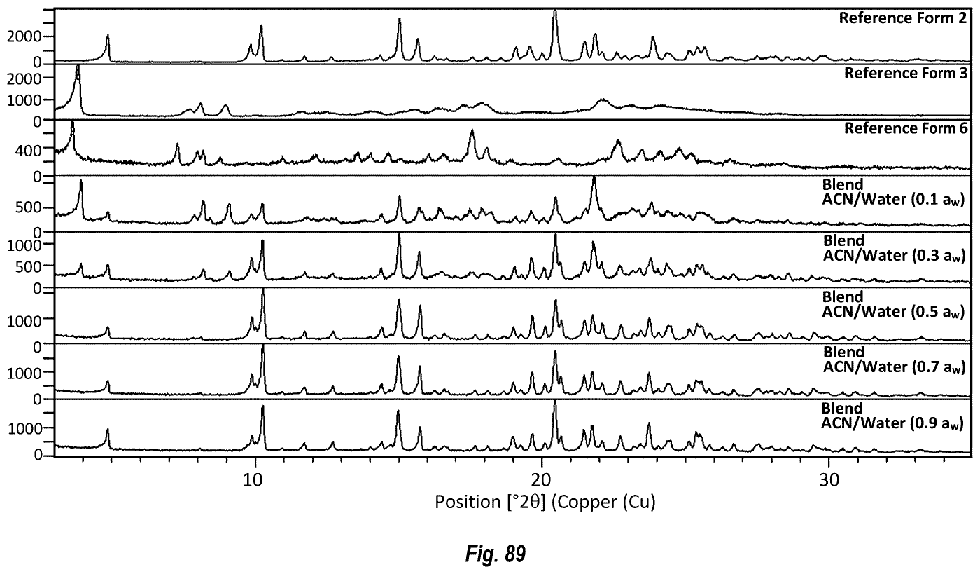

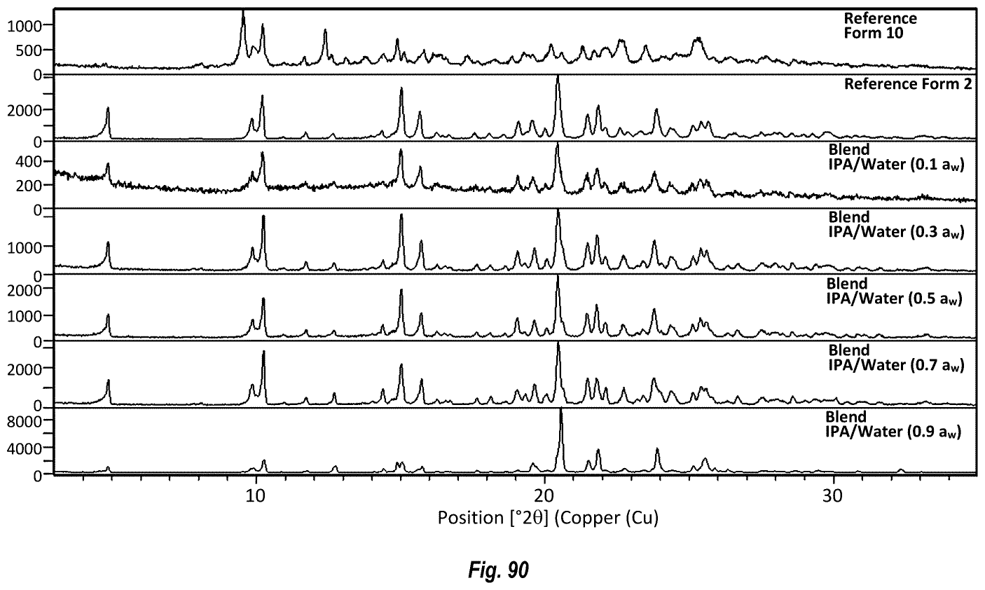

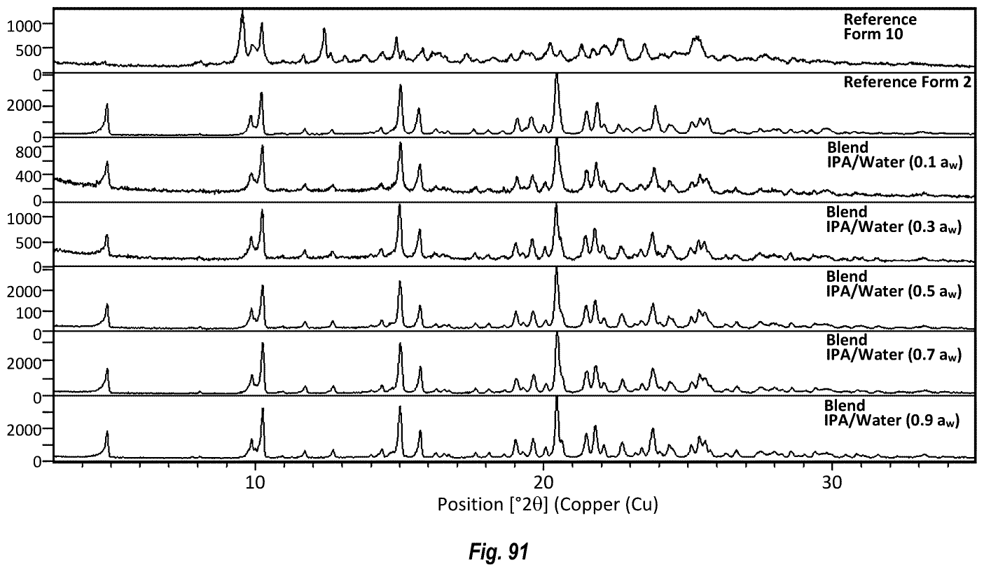

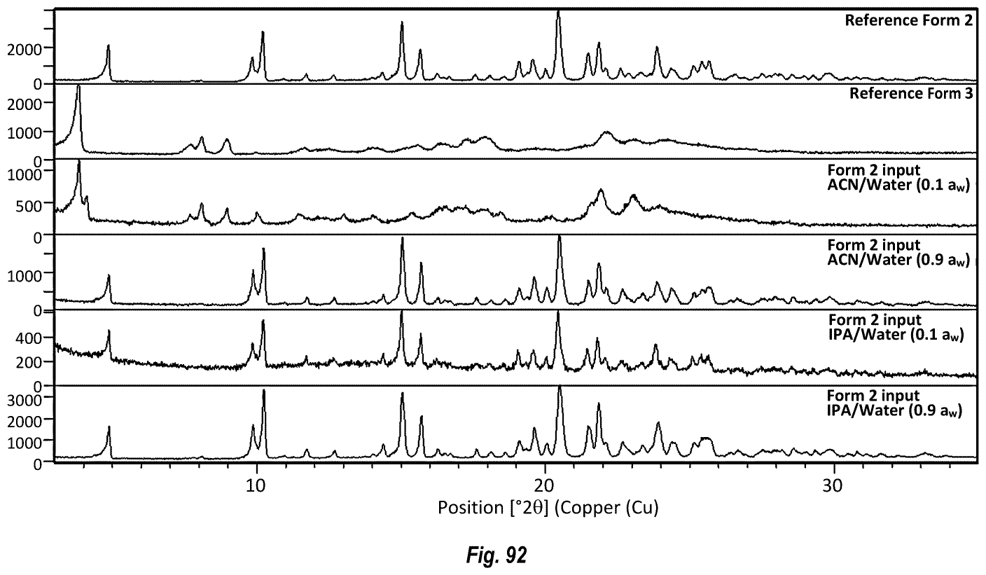

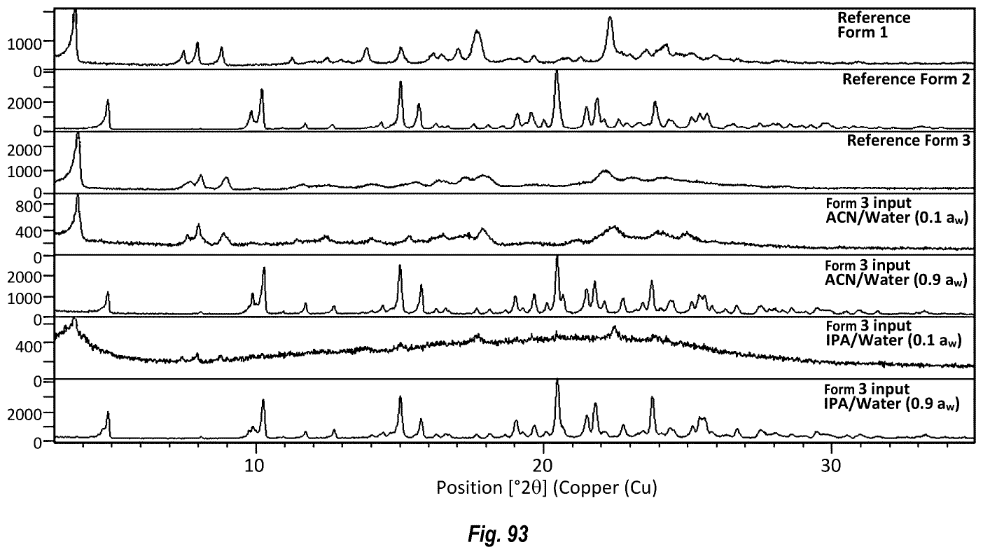

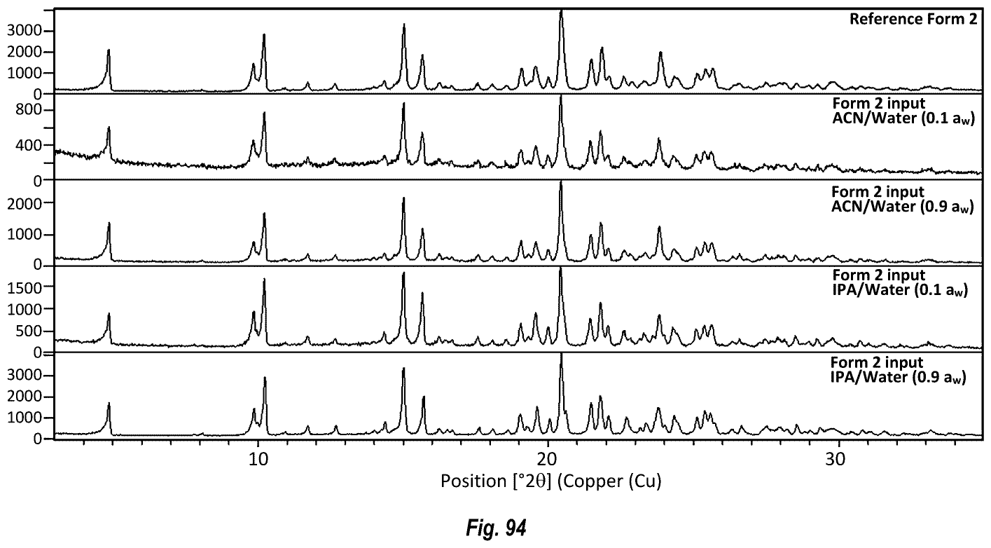

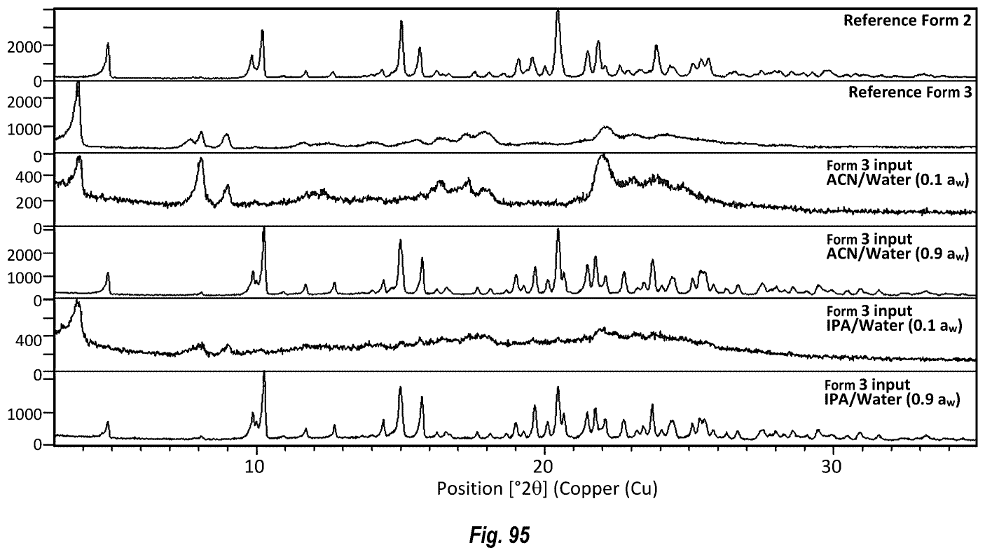

XRPD diffraction overlay associated with the hydration mapping studies are set forth in FIGS. 80-95.

Example 18-Seven Day Stability Study on Crystalline Form 2 Compound 1 di(hydrogen sulfate) trihydrate: A 7-day stability study was conducted on crystalline Form 2 Compound 1 di(hydrogen sulfate) trihydrate using the following conditions: (1) ambient light, temperature, and humidity conditions (open vial); (2) 40° C., 75% RH (open vial); and (3) 80° C. (closed vial). After 7 days of storage under the specified, XRPD and HPLC analyses were carried out on the residual solid materials. The results are summarized in Table 58 below.

TABLE 58

| Conditions | XRPD | HPLC (% area) |
|---|---|---|
| Input Sample | 2 | 99.92 |
| Ambient | 2 | 99.86 |
| 40° C./75% RH | 2 | 99.86 |
| 80° C. | 2 | 99.85 |

Example 19-Solubility of Crystalline Form 2 Compound 1 di(hydrogen sulfate) trihydrate: The thermodynamic solubility of crystalline Form 2 Compound 1 di(hydrogen sulfate) trihydrate was determined in 8 aqueous media (0.1M HCl, Phthalate buffer pH 3, acetate buffer pH 4.2, phosphate buffer pH 7, phosphate buffer pH 7.4, alkaline borate buffer pH 9.0, purified water EP/USP and phosphate water pH 12). Buffer solutions were prepared according to the United States Pharmacopeia USP 43 with modified buffer strength. Suspensions were prepared by addition of a single aliquot of the aqueous medium to the crystalline Form 2 Compound 1 di(hydrogen sulfate) trihydrate. The pH was titrated to the targeted pH with 1M NaOH. After incubation for 24 hours at 37° C., the pH was measured, and the mother liquors were filtered and analyzed by UPLC to determine the concentration of the free base. The residual solids after the solubility determination were ambient- and vacuum dried and analyzed by HT-XRPD.

The solubility of crystalline Form 2 Compound 1 di(hydrogen sulfate) trihydrate was determined in aqueous media. A strong dependence upon pH was observed. At low pH values (<3.0), crystalline Form 2 Compound 1 di(hydrogen sulfate) trihydrate was freely soluble; at pH 4.2, crystalline Form 2 Compound 1 di(hydrogen sulfate) trihydrate was soluble; at pH 7.0 crystalline Form 2 Compound 1 di(hydrogen sulfate) trihydrate was slightly soluble; and at high pH (12.0) crystalline Form 2 Compound 1 di(hydrogen sulfate) trihydrate was very slightly soluble.

Crystalline Form 2 Compound 1 di(hydrogen sulfate) trihydrate was found to be more soluble than the free base. At pH 9, crystalline Form 2 Compound 1 di(hydrogen sulfate) trihydrate in solution is the free base.

Example 20-ENT1 Activity of Crystalline Form 2 Compound 1 di(hydrogen sulfate) trihydrate compared to Compound 1 (free base)-Initial and after Six-Month Storage: (1) Initial time: Whole blood from 8 healthy donors was incubated with 5-Ethynyl-Uridine (5EU, CAS #: 69075-42-9, Jena Bioscience) for 60 min at 37° C., in the absence or presence of freshly suspended 1 μM Compound 1 (free base) or 1 μM Form 2 Compound 1 di(hydrogen sulfate) trihydrate. Intracellular uptake of 5EU through ENT1 transporters was assessed by standard flow cytometry and ENT1 activity was determined.

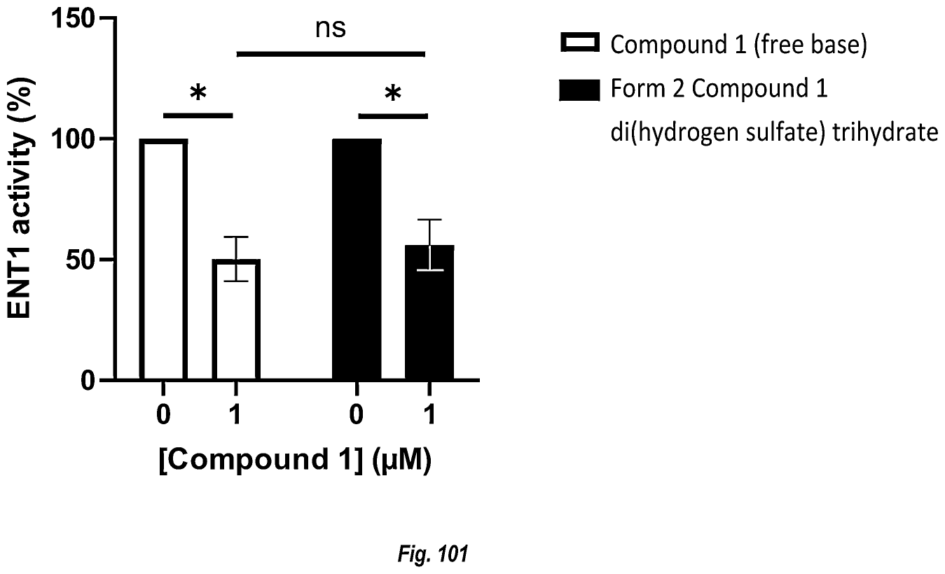

As shown in FIG. 101, ENT1 activity significantly decreased by 49.8% or 43.9% compared to untreated CD19 positive cells in presence of Compound 1 (free base) or Form 2 Compound 1 di(hydrogen sulfate) trihydrate, respectively. No significant difference in ENT1 inhibition levels was evidenced by either treatment, showing that Form 2 Compound 1 di(hydrogen sulfate) trihydrate and Compound 1 (free base) have essentially the same inhibitory potency.

In FIG. 101, the data are expressed as % of ENT1 activity in untreated CD19 cells, arbitrary set at 100%, and are the means±SD of three independent experiments. Statistical analysis was performed using one-way analysis of variance (ANOVA) followed by Tukey's post hoc analysis with Prism 10.1.2 software (GraphPad Software). *: p<0.0001 vs. untreated B cells; ns: non-significant.

(2) Six months: Compound stability was evaluated on suspended solutions of Compound 1 (free base) or Form 2 Compound 1 di(hydrogen sulfate) trihydrate that were stored at −20° C. for 6 months. On the day of the experiment, whole blood from 3 healthy donors was incubated with 5-Ethynyl-Uridine (5EU, CAS #: 69075-42-9, Jena Bioscience) for 60 min at 37° C., in the absence or presence of either 1 μM Compound 1 (free base) or 1 μM Form 2 Compound 1 di(hydrogen sulfate) trihydrate of 6-month-old solutions. Intracellular uptake of 5EU through ENT1 transporters was assessed by standard flow cytometry and ENT1 activity was determined.

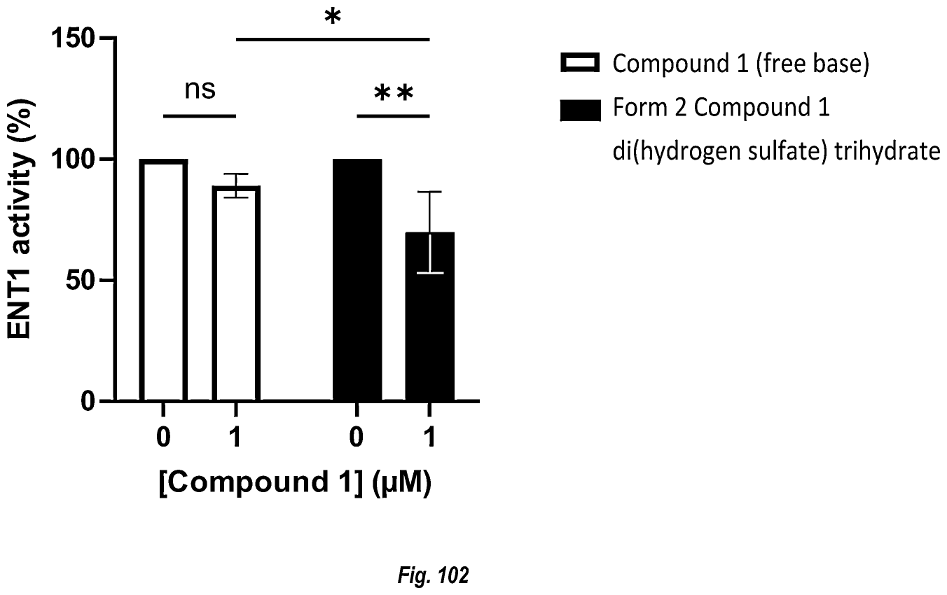

As shown in FIG. 102, ENT1 activity was significantly decreased by 30.4% compared to untreated CD19 positive cells in presence of Form 2 Compound 1 di(hydrogen sulfate) trihydrate. No significant effect of Compound 1 (free base) on ENT1 inhibition levels was evidenced. Taken together, these data suggest that Compound 1 (free base) is degrades in potency more than Form 2 Compound 1 di(hydrogen sulfate) trihydrate in suspended solutions after 6 months of long-term stability evaluation.

In FIG. 102, the data are expressed as % of ENT1 activity in untreated CD19 cells, arbitrary set at 100%, and are the means±SD. Statistical analysis was performed using two-way analysis of variance (ANOVA) with Prism 10.1.2 software (GraphPad Software). *: p<0.05 vs. untreated B cells; **: p<0.01 vs. untreated B cells; ns: non-significant.

Summary of certain data on different crystalline Compound 1 hydrogen sulfates: Table 59 provides a summary of data from crystalline Form 2 Compound 1 di(hydrogen sulfate) trihydrate, crystalline Form 3 Compound 1 di(hydrogen sulfate), crystalline Form 4 Compound 1 mono(hydrogen sulfate), and crystalline Form 8 Compound 1 mono(hydrogen sulfate).

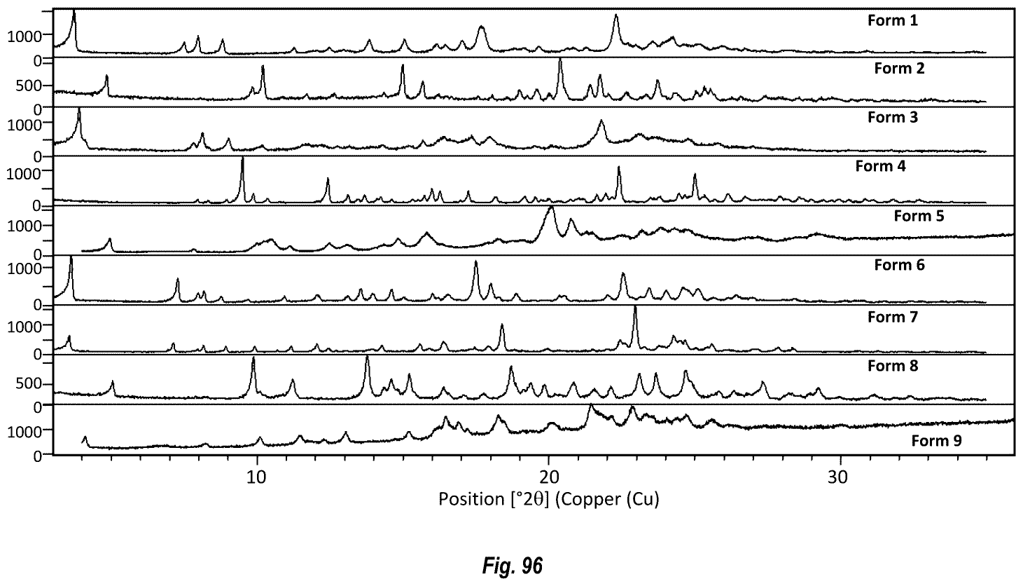

A comparison of XRPD patterns is shown in FIG. 96.

TABLE 59

| | Crystalline | | | |
|---|---|---|---|---|
| | Form 2 | Crystalline | Crystalline | Crystalline |
| | Compound 1 | Form 3 | Form 4 | Form 8 |
| | di(hydrogen | Compound 1 | Compound 1 | Compound 1 |
| | sulfate) | di(hydrogen | mono(hydrogen | mono(hydrogen |
| Characterization | trihydrate | sulfate) | sulfate) | sulfate) |
| XRPD | Crystalline | Partially | Crystalline | Crystalline |
| | | Crystalline | (some shifts | |
| | | | upon air drying) | |
| TGA/DSC | 7.3% mass loss | 3.1% mass loss | 15.5% mass loss | 7.2% mass loss |
| | 130° C. (149° C.) | 163° C. (177° C.) | 148° C. (160° C.) | 144° C. (158° C.) |
| DSC | 144° C. (158° C.) | 133° C. (139° C.), | 113° C. (119° C.) | x |
| (onset/peak) | | 165° C. (169° C.) | | |
| DVS | 8.0% uptake at | 7.3% uptake at | 8.1% uptake at | x |
| | 80% RH | 80% RH | 40-50% RH | |
| | Dehydrates <15% RH | Conversion to P2 | (2^{nd} sorption) | |
| | | | 8.0% loss at | |
| | | | 20-30% RH | |
| | | | (desorption) | |
| | | | Conversion to | |
| | | | Form 8 after | |
| | | | DVS at 40% RH | |
| VH-XRPD | Conv. To Form | Conv. To Form 6→ | x | x |
| | 5 at 6% RH | Form 2 at ≥70% RH | | |
| | Form 2 retained | Form 3 retained | | |
| | at 10-80% RH | at 10-60% RH | | |
| VT-XRPD | Form 2 retained | Form 3 retained | Conv. To Form 8 | x |
| | at 30-50° C. | at 25-50° C. | at 40-70° C. | |
| | Conv. To Form 5 | Conv. To Form 9 | Conversion to | |
| | at 80-130° C. | from 50-160° C. | amorph. At ≥90° C. | |
| | Melt from 150° C. | Melt from 160° C. | | |
| FT-IR* | For reference | For reference | For reference | For reference |
| NMR | 0.73% heptane; | No residual | Peak shifts | Peak shifts |
| | 1.04% IPA | solvent observed | observed, compared | comparable to |
| | | | to Form 2 | Form 4 |
| HPLC (purity) | 99.92% area | 99.83% area | 99.66% area | x |
| HPLC-CAD* | 1.8 equiv. | 1.86 equiv. | 0.85 equiv. | 0.84 equiv. |
| | of acid | of acid | of acid | of acid |
| KF | 6.74% moisture | 1.89% moisture | 14.87% moisture | ~ |
| | content | content | content | |
| 7-day Stability | No change in | Form 6 at ambient; | Form 4 retained | x |
| studies | solid form for | Form 2 at | at all conditions | |
| | all conditions | 40° C./75% RH; | (crystallinity | |
| | Purity similar | Form 9 at 80° C. | significantly | |
| | to input | Purity similar | reduced at 80° C.) | |
| | | to input | Purity similar | |
| | | | to input | |
| pH solubility | pH 1.2: >87 | pH 1.2: 76 | pH 1.2: 134 | x |
| | mg/mL, no solid | mg/mL, Form 2 | mg/mL, Form 4 | |
| | pH 4.5: 95 | pH 4.5: 54 | pH 4.5: 47 | |
| | mg/mL, Form 4 | mg/mL, Form 4 | mg/mL, Form 4 | |
| | pH 6.8: 12 | pH 6.8: 150 | pH 6.8: 62 | |
| | mg/mL, Gum | mg/mL, Gum | mg/mL, Gum | |
| Nature of Form | Di(hydrogen | Di(hydrogen | Mono(hydrogen | Mono(hydrogen |
| | sulfate) | sulfate) hydrate | sulfate) hydrate | sulfate) hydrate |
| | Trihydrate | | | |

x = Analysis not carried out.
* = FT-IR and HLPC-CAD are routine techniques. CAD (Charged aerosol detector) may be used in conjunction with HPLC (HPLC-CAD) to measure the amount of chemicals in a sample by creating charged aerosol particles for detection.

### Examples—Hydrocloric Acid, P-Toluenesulfonic Acid, Methanesulfonic Acid, Benzenesulfonic Acid, Phosphoric Acid, Maleic Acid, L-Tartaric Acid, Fumaric Acid, Citric Acid, L-Malic Acid, Benzoic Acid

### Example 21: Analytical Methods

### X-Ray Powder Diffraction (XRPD)

In the examples below, XRPD analysis was carried out on a PANalytical X′pert pro with PIXcel detector (128 channels), scanning the samples between 3 and 35° 2θ. The material was gently ground (where required) to release any agglomerates and loaded onto a multi-well plate with Mylar polymer film to support the sample. The multi-well plate was then placed into the diffractometer and analyzed using Cu K radiation (α₁ λ=1.54060 Å; α₂=1.54443 Å; β=1.39225 Å; α₁:α₂ ratio=0.5; most, if not all, β radiation is removed from the beam using an X-ray mirror) running in transmission mode (step size 0.0130° 2θ, step time 18.87 s) using 40 kV/40 mA generator settings. Data were visualized and images generated using the HighScore Plus 4.7 desktop application (PANalytical, 2017).

### Variable Humidity X-Ray Powder Diffraction (VH-XRPD)

VH-XRPD analysis was carried out on a Philips X'Pert Pro Multipurpose diffractometer equipped with a humidity chamber. The samples were scanned between 4 and 36° 2θ using Cu K radiation (α₁ λ=1.54060 Å; α₂=1.54443 Å; β=1.39225 Å; α₁:α₂ ratio=0.5; most, if not all, β radiation is removed from the beam using an X-ray mirror) running in Bragg-Brentano geometry (step size 0.008° 2θ) using 40 kV/40 mA generator settings. Measurements were carried out from 6-90% relative humidity (RH), with a final measurement taken at 40% RH.

### Variable Temperature X-Ray Powder Diffraction (VT-XRPD)

VT-XRPD analysis was carried out on a Philips X'Pert Pro Multipurpose diffractometer equipped with a temperature chamber. The samples were scanned between 4 and 36° 2θ using Cu K radiation (α₁ λ=1.54060 Å; α₂=1.54443 Å; β₂=1.39225 Å; α₁:α₂ ratio=0.5; most, if not all, β radiation is removed from the beam using an X-ray mirror) running in Bragg-Brentano geometry (step size 0.008° 2θ) using 40 kV/40 mA generator settings. Measurements were carried out from ambient temperature to an upper limit of 250° C., with a final measurement taken after cooling.

### Polarised Light Microscopy (PLM)

The presence of crystallinity (birefringence) was determined using an Olympus BX53 polarizing microscope, equipped with a Motic camera. Images were captured using Motic Images Plus 3.0. All images were recorded using the 20× objective, unless otherwise stated.

### Thermogravimetric/Differential Scanning Calorimetry (TG/DSC)

2-10 mg of material was added into a pre-tared open aluminum pan and loaded into a TA Instruments Discovery SDT 650 Auto-Simultaneous DSC and held at room temperature. The sample was then heated at a rate of 10° C./min from 30° C. to 400° C. during which time the change in sample weight was recorded along with the heat flow response (DSC). Nitrogen was used as the sample purge gas, at a flow rate of 200 cm³/min.

### Differential Scanning Calorimetric Analysis (DSC)

1-5 mg of material was weighed into an aluminum DSC pan and sealed non-hermetically with a pierced aluminum lid. The sample pan was then loaded into a TA DSC2500 and held at 20° C. Once a stable heat-flow response was obtained, the sample and reference were heated to an upper temperature of 200° C. at a scan rate of 10° C./min and the resulting heat flow response monitored. The sample was held at the upper temperature for 3 min, before it was cooled at 10° C./min to 20° C., reheated to the upper temperature at 10° C./min, and then cooled again to 20° C. at the same rate. Nitrogen was used as the purge gas, at a flow rate of 50 cm³/min.

### Nuclear Magnetic Resonance Spectroscopy (NMR)

¹H NMR spectroscopic experiments were performed on a Bruker AV500 (frequency: 500 MHz for protons). Experiments were performed in d₄-methanol and samples were prepared to ca. 5 mM concentration.

### Dynamic Vapor Sorption (DVS)

Approximately 10-20 mg of sample was placed into a mesh vapor sorption balance pan and loaded into either a DVS Intrinsic or DVS Advantage dynamic vapor sorption balance by Surface Measurement Systems. The sample was subjected to a ramping profile from 40-90% RH at 10% increments, maintaining the sample at each step until a stable weight had been achieved (dm/dt 0.004%, minimum step length 30 min, maximum step length 500 min) at 25° C. After completion of the sorption cycle, the sample was dried using the same procedure to 0% RH and then a second sorption cycle back to 40% RH was carried out. Two cycles were performed. The weight change during the sorption/desorption cycles were plotted, allowing the hygroscopic nature of the sample to be determined.

### High Performance Liquid Chromatography-Ultraviolet Detection (HPLC-UV)

Column: Waters XSelect CSH Fluoro-Phenyl 150×4.6 mm, 2.5 μm
Mobile Phase A: 0.1% TFA in water
Mobile Phase B: 0.1% TFA in acetonitrile
Diluent: Methanol
Autosampler Temperature: 5° C.
Flow Rate: 1.0 mL/min
Runtime: 30 min
Column Temperature: 30° C. (±1° C.)
Column Pressure: 110 bar (at start of run)
Injection Volume: 5 μL
Sample Concentration: 0.5 mg/mL
Detection: 266 nm
Sampling Rate: 50 Hz
Gradient Program:

| Time/min | Mobile Phase A (%) | Mobile Phase B (%) |
|---|---|---|
| 0.0 | 95 | 5 |
| 5.0 | 75 | 25 |
| 18.0 | 65 | 35 |
| 20.0 | 50 | 50 |
| 22.0 | 5 | 95 |
| 25.0 | 5 | 95 |
| 25.1 | 95 | 5 |
| 30.0 | 95 | 5 |

### Liquid Chromatography-Mass Spectrometry (LCMS)

LCMS was preformed using one of the following methods:

### Method A:

Instrument: Agilent 1200 HPLC MSD:6120 single quadrupole MSD
Column: Luna C18, 2.0*50 mm, 5 μm
Column temperature: 40° C.
Mobile Phase A (MP A): 0.04% TFA in H₂O
Mobile Phase B (MP B): 0.02% TFA in ACN
Flow Rate: 1.0 mL/min
Detection: 220 nm
Gradient Ratio:

| Time (min) | MP A (%) | MP B (%) |
|---|---|---|
| 0.01 | 95 | 5 |
| 0.40 | 95 | 5 |
| 3.00 | 5 | 95 |
| 4.00 | 5 | 95 |
| 4.01 | 95 | 5 |
| 4.50 | 95 | 5 |

### Method B:

Instrument: Shimadzu LC-20AD MSD:LCMS-2020
Column: Kinetex 5 um EVO C18 30*2.1 mm
Column temperature: 40° C.
Mobile Phase A (MP A): 0.04% TFA in H₂O
Mobile Phase B (MP B): 0.02% TFA in CAN
Flow Rate: 1.5 mL/min

| Time (min) | MP B (%) | Flow (ml/min) |
|---|---|---|
| 0.01 | 5 | 1.5 |
| 0.70 | 95 | 1.5 |
| 1.16 | 95 | 1.5 |
| 1.50 | 5 | 1.5 |

### Chiral High-Performance Liquid Chromatography (Chiral HPLC)

Chiral HPLC was preformed using one of the following methods:

### Method 1:

Instrument: CAS-TJ-Chiral HPLC-K (Waters Arc with PDA detector)
Proc. Chnl. Descr.: 2998 PDA 254.0 nm (2998 (190-300)nm)
Column: Chiralpak IC-3, 50×4.6 mm, I.D., 3 um
Mobile phase: A: Heptane B: EtOH (0.05% DEA, v/v)
Gradient: A:B=20:80
Flow rate: 1 mL/min
Column temp.: 35° C.

### Method 2:

Instrument: CAS-TJ-ANA-Chiral HPLC-K (Waters Arc with 2998)
Proc. Chnl. Descr.: 2998 PDA 254.0 nm (2998 (190-300)nm)
Column: Chiralpak IF-3, 150×4.6 mm I.D., 3 um
Mobile phase: A: Heptane B: EtOH+ACN (4:1) (0.05% IPAm, v/v)
Gradient: A:B=92:8
Flow rate: 1 mL/min
Column temp.: 30° C.

### Example 22. Salt Preparation Methods

Salt Preparation Method 1: Approximately 25 mg of Compound 1 (free base) was weighed out into 1.5 mL screw-cap vials and a stirrer bar was added. For solid counterions, the appropriate amount of acid (1.0 or 2.0 equiv.) was weighed into separate 1.5 mL screw-cap vials. 100 μL of the appropriate solvent system was added to both the Compound 1 and the solid counterions. Stock solutions of liquid counterions were prepared, such that 100 μL contained 1.0 or 2.0 equiv. of acid. See counterion and solvent lists in Table 60 and Table 61 respectively.

The samples were stirred at 40° C. for 5-10 min and then the acid solutions were added to Compound 1, rinsing out the transfer vial with a further 50 μL of solvent. If acid was observed to be insoluble in solvent system, the Compound 1 solution was added to acid slurry. Experiments were temperature cycled between 40° C. and 5° C. (1 h hold; 0.1° C./min ramp/cool) for ca. 24-68 h. Anti-solvent addition (ASA) was carried out to slurries at 5° C. then experiments were heated to 30° C. over 1 h. ASA was carried out at 30° C. or 40° C. where clear solutions or gums were observed.

Temperature cycling was then continued for ca. 16-69 h, followed by further ASA and temperature cycling if required. Material was isolated at 5° C. and analyzed damp by XRPD. All isolated material was dried under vacuum at ca. 40° C. for ca. 17-91 h then re-analyzed by XRPD. The XRPD plate was stored at 40° C./75% RH for 24-48 h and then samples re-analyzed. Solutions were stored uncapped at ambient temperature to evaporate. Any residual solids were analyzed by XRPD.

TABLE 60
List of counterions assessed in Salt Preparation Method 1

| Counterions | 1.0 equiv. | 2.0 equiv. |
|---|---|---|
| Hydrochloric acid | ✓ | ✓ |
| p-Toluenesulfonic acid | ✓ | ✓ |
| Methanesulfonic acid | ✓ | ✓ |
| Benzenesulfonic acid | ✓ | ✓ |
| Phosphoric acid | ✓ | ✓ |
| Maleic acid | ✓ | NA |
| L-Tartaric acid | ✓ | NA |
| Fumaric acid | ✓ | NA |
| Citric acid | ✓ | NA |
| L-Malic acid | ✓ | NA |
| Benzoic acid | ✓ | NA |

NA = not applicable

TABLE 61
Solvent systems assessed in Salt Preparation Method 1
Solvent Systems

Acetonitrile
Ethanol
Ethyl acetate
MEK
THF
2-propanol:water (75:25% v/v)

Salt Preparation Method 2: The general procedure adopted in preparing the potential salts is outlined below. A known mass of Compound 1 (free base) and a known volume of the appropriate solvent system were added to 20 mL scintillation vials. The required mol. equiv. of the appropriate counterions were added to the vials as solutions or slurries in the corresponding solvent system.

The experiments were stirred at 40° C. for ca. 1 h, and then temperature cycled between 40° C. and 5° C. with 0.1° C./min ramp and 1 h hold between each step. Anti-solvent additions and subsequent slurrying at 5° C. were employed in order to facilitate crystallization. When slurries were observed in the vials, the solids were isolated via vacuum filtration and dried under vacuum at ca. 40° C. for 24-48 h. The damp and dried solids were subsampled and analyzed by XRPD. Where applicable, the dried solids were stored at 40° C./75% RH for 24-48 h in order to improve the crystallinity and/or facilitate solid form conversion to the desired form.

### Example 23. Hydrochloric Acid

Example 23A—Prior to acid addition, complete dissolution of Compound 1 (free base) was observed in all the solvent systems assessed except acetonitrile where partial dissolution was observed instead. Following Salt Preparation Method 1 and using 1 mol. equiv. of hydrochloric acid, the isolated materials from ethyl acetate, MEK, and THF were observed to be gum-like. Glassy solid was obtained from acetonitrile, ethanol, and 2-propanol: water (75:25% v/v). All the solids analyzed were found to be amorphous, as noted below in Table 62.

TABLE 62
Salt Preparation Method 1 - Hydrochloric acid, 1 equiv.

| | | XRPD Analysis | | |
|---|---|---|---|---|
| Initial Solvent System | AS | Damp | Dry | 40° C. 75% RH |
| Acetonitrile | tBME | A | A | A |
| | | (evap.) | (evap.) | |
| Ethanol* | Heptane | A | A | A |
| | | (evap.) | (evap.) | |
| Ethyl acetate | Heptane | A | A | A |
| MEK | Heptane | A | A | A |
| THF | Heptane | A | A | A |
| IPA:H₂O (75:25% v/v) | tBME | A | A | A |
| | | (evap.) | (evap.) | |

*50 μL of methanol was added after the second temperature cycling to homogenize biphasic solutions
A = amorphous
Evap. = isolated via evaporation

Example 23B—Prior to acid addition, complete dissolution of Compound 1 (free base) was observed in all the solvent systems assessed. Following Salt Preparation Method 1 and using 2 mol. equiv. of hydrochloric acid, the isolated materials from acetonitrile and ethyl acetate were observed to be sticky solids. Solid was obtained from ethanol. Gum-like material was obtained from MEK and THF. Oil was obtained from 2-propanol:water (75:25% v/v). All solvent systems analyzed were found to give amorphous material except ethanol, as noted below in Table 63.

TABLE 63
Salt screen - Hydrochloric acid, 2 equiv.

| | | XRPD Analysis | | |
|---|---|---|---|---|
| Initial Solvent System | AS | Damp | Dry | 40° C. 75% RH |
| Acetonitrile | tBME | A | A | A |
| Ethanol* | Heptane | 1 | 1 | 1 (PC) |
| Ethyl acetate | Heptane | A | A | A |
| MEK | Heptane | A | A | A |
| THF | Heptane | A | A | A |
| IPA:H₂O (75:25% v/v) | tBME | A | A | A |

*Slurry formed after further ASA and temperature cycling
A = amorphous
1 = Form 1 Compound 1 hydrogen chloride
PC = poorly crystalline

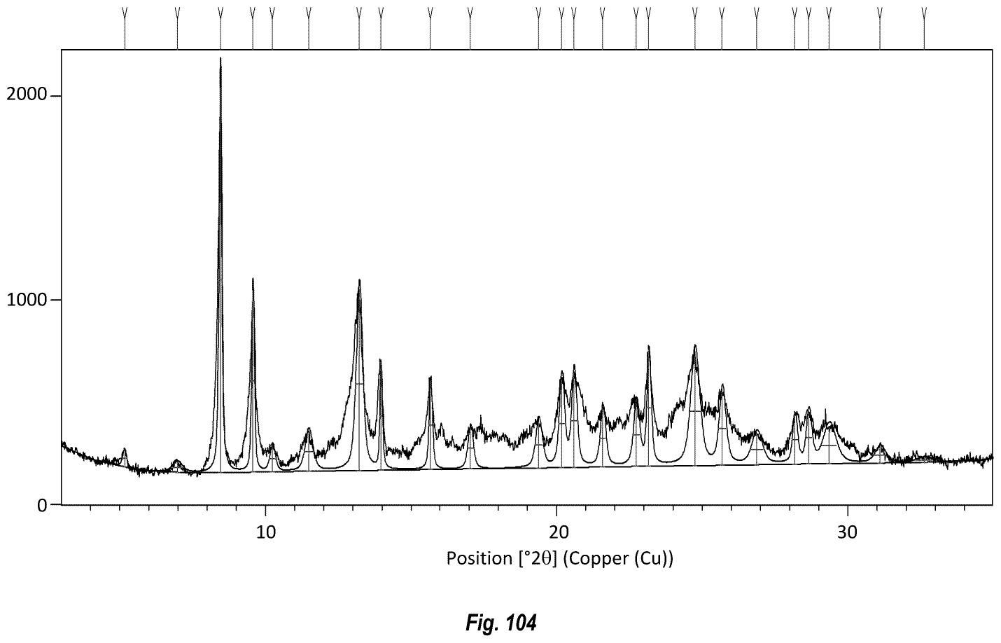

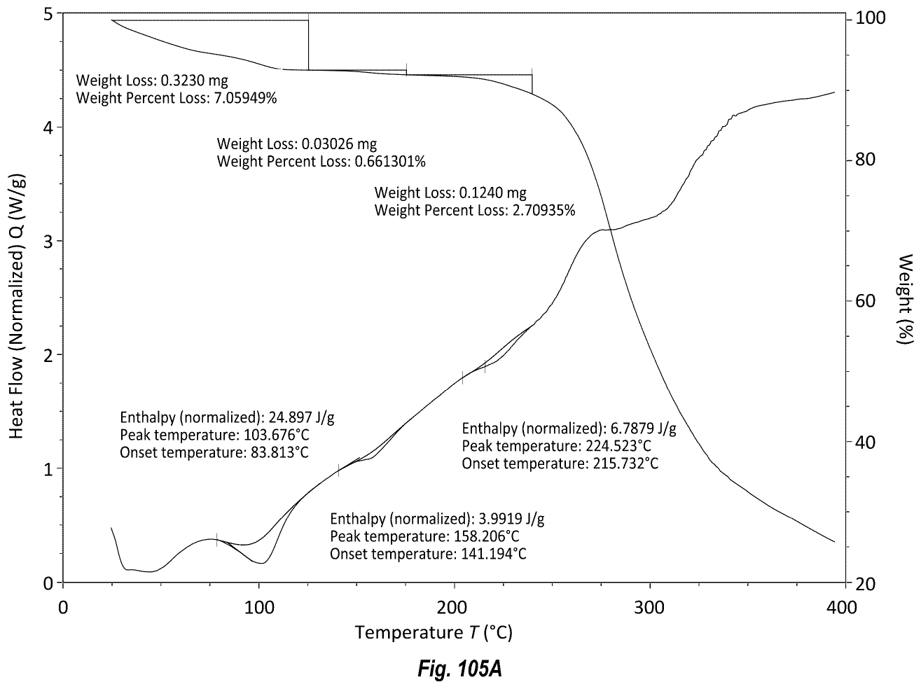

The XRPD patterns of crystalline Form 1 Compound 1 hydrogen chloride are shown in FIGS. 103 and 104. The TG/DSC of the dried sample from ethanol showed 7.1% mass loss between outset −125° C., followed by a further mass loss of 0.7% between 125-175° C. See FIG. 105A. Endothermic events were observed at peak onset 84° C. (peak at 104° C.), peak onset 141° C. (peak at 158° C.), and peak onset 216° C. (peak at 225° C.). The ¹H-NMR spectrum was consistent with the structure of the molecule, with peak shifting observed when compared with Compound 1 (an indication of salt formation). Peaks corresponding to <0.1% w/w ethanol and 0.7% w/w heptane were observed in the spectrum.

Example 23C—Amorphous Compound 1 hydrogen chloride and crystalline Form 1 Compound 1 hydrogen chloride were also prepared following the general procedure outlined in Salt Preparation Method 2. More specifically, amorphous Compound 1 hydrogen chloride was prepared as follows. A known mass of Compound 1 (free base), 2.05 mol. equiv. of HCl, and 8 vol. of ethanol were added to a 20 mL scintillation vial. The experiment was stirred at 5° C. and a clear solution was obtained. 6 vol. of anti-solvent (heptane) was added to 5° C. and sticky solids were observed after stirring at 5° C. for 24 h. The solids were isolated via vacuum filtration and dried under vacuum at ca. 40° C. for 24 h. The damp and dried solids subsampled and analyzed by XRPD. The dried solid was found to be amorphous by XRPD.

Crystalline Form 1 Compound 1 hydrogen chloride was prepared as follows. A known mass of Compound 1 (free base) and 5 vol. of ethanol were added to a 20 mL scintillation vial. 2.05 mol. equiv. of hydrochloric acid was added to the vial as a solution in 5 vol. of ethanol. The sample was stirred at 40° C. for ca. 1.5 h and a clear solution was observed; 16 vol. of anti-solvent (heptane) was then added to the vial and stirring continued at 40° C. for another 2 h. Gum formation was observed. The experiment was then cooled to 5° C. at 0.1° C./min and left stirring at 5° C. for ca. 16 h. Slurry formation was observed. Afterwards, the solids were isolated via vacuum filtration and dried under vacuum at ca. 40° C. for 48 h. The damp and dried solids were subsampled and analyzed by XRPD. The dried solids were characterized as discussed below.

Characterization of the prepared crystalline Form 1 Compound 1 hydrogen chloride yielded the following results.

PLM images showed slightly birefringent particles with no clear morphology.

TG/DSC analysis showed stepwise mass losses of ca. 8.4% between 20° C.-140° C., which corresponded with two endothermic events at peak onsets 34° C. and 91° C. (peaks at 48° C. and 105° C. respectively). These are likely due to solvent/water loss (theoretically, 8.4%=3.6 mol. equiv. H₂O). See FIG. 105B. A small endothermic event was observed at peak onset 167° C. (peak at 182° C.). This is likely due to a melting event. The thermal events observed above 250° C. are likely due to degradation.

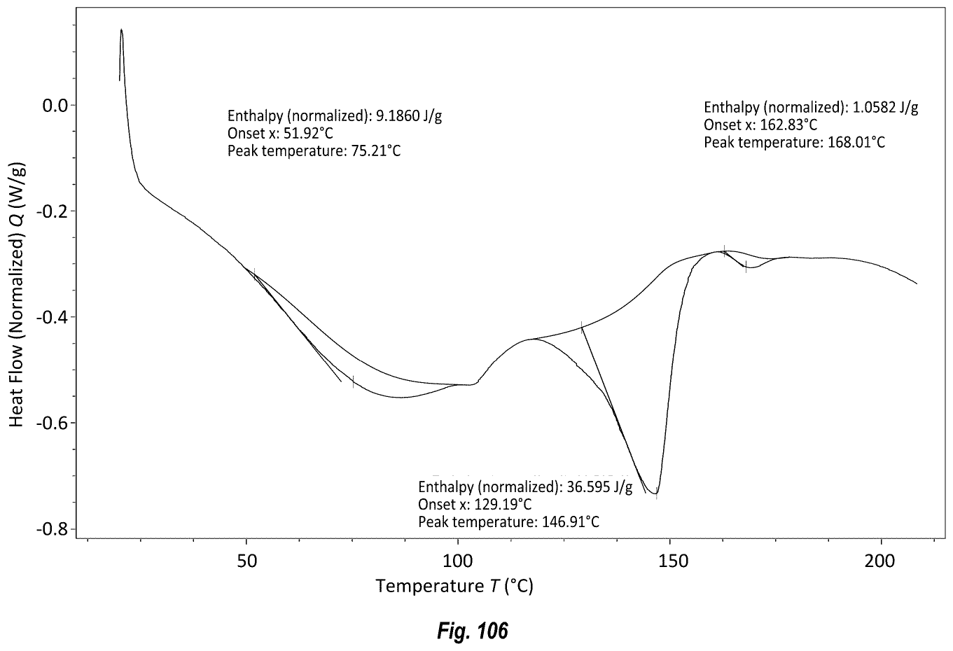

DSC analysis showed an endothermic event observed at peak onset 52° C. (peak at 75° C.), which is likely due to solvent/water loss. See FIG. 106. Two subsequent endothermic events were observed at peak onsets 129° C. and 163° C. (peaks at 147° C. and 168° C.). These are likely due to further solvent loss and subsequent melting event.

¹H-NMR spectrum was consistent with the received structure and showed peak shifts with respect to the Compound 1 material (indicating salt formation). Peaks corresponding to 0.07% w/w ethanol were observed.

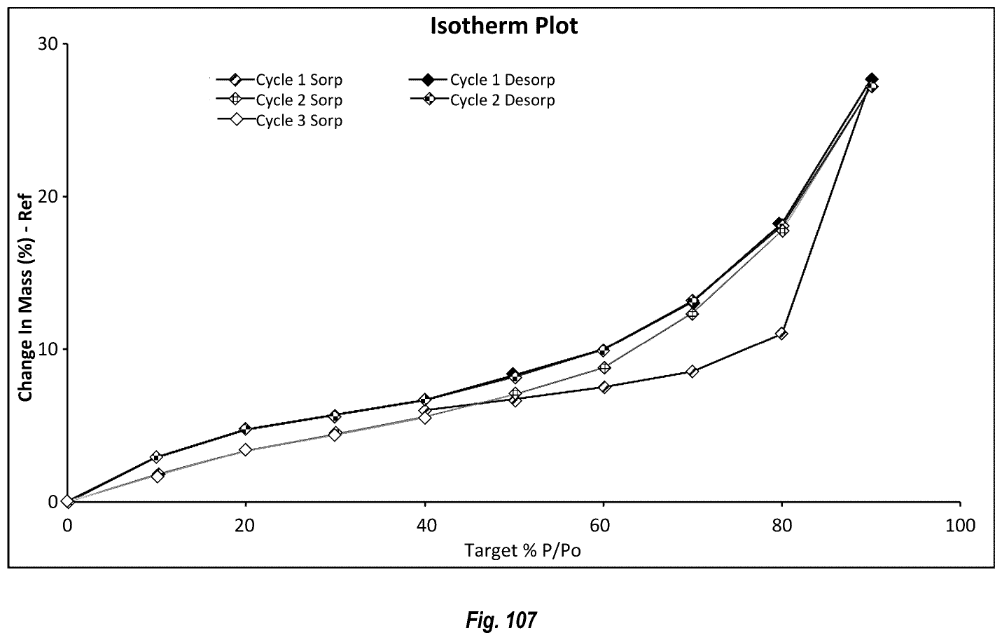

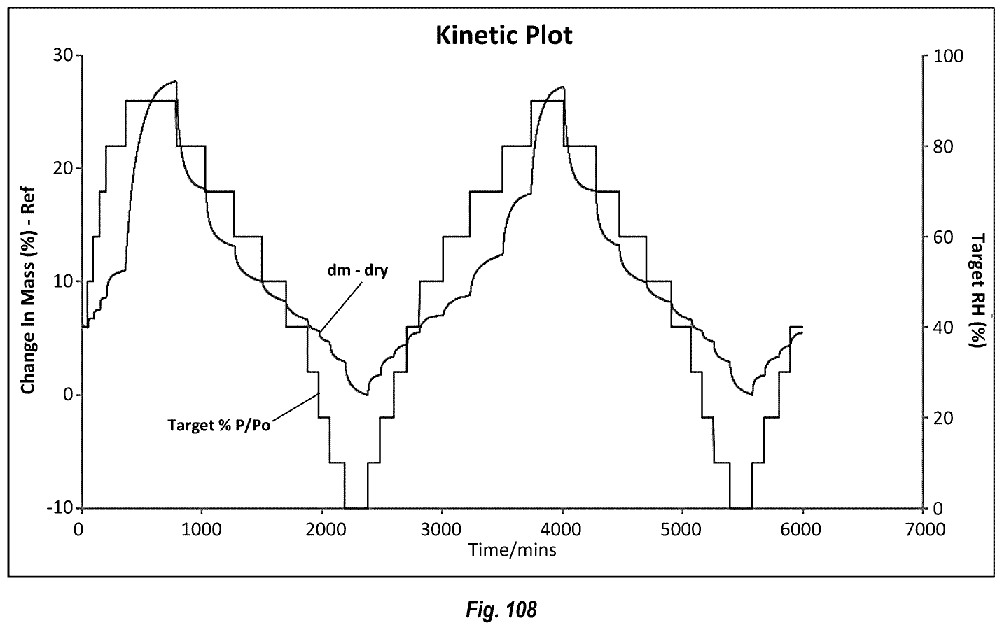

DVS analysis showed a moisture uptake of ca. 5% between 40-80% RH during the first sorption cycle and an uptake of 18% was observed between 0-80% RH during the second sorption cycle, indicating moderate-high hygroscopicity. See FIG. 107 and FIG. 108. A further uptake of 9% was observed between 80-90% RH. This is likely due to the sample deliquescing at high humidity conditions. Hysteresis of ca. 1.3% was observed between the sorption and desorption cycles. The post-DVS sample was observed to be gum-like material further indicating prior deliquescing.

HPLC analysis indicated a solid purity of 99.31% area.

HPLC-CAD indicated the presence of 2.09 mol. equiv. of chloride.

VH-XRPD analysis carried out between 7-80% RH showed crystalline Form 1 Compound 1 hydrogen chloride being retained throughout. See detailed results in Table 64.

TABLE 64
VH-XRPD analysis for crystalline Form
1 Compound 1 hydrogen chloride

| | Rel. Humidity | Hold time | XRPD |
|---|---|---|---|
| Cycle | (% RH) | (min) | Results |
| Desorption | 40 | 60 | 1 |
| | 20 | 960 | 1 |
| | 10 | 60 | 1 |
| | 7 | 60 | 1 |
| Sorption | 10 | 60 | 1 |
| | 20 | 60 | 1 |
| | 40 | 60 | 1 |
| | 60 | 60 | 1 |
| | 70 | 60 | 1 |
| | 80 | 60 | 1 |
| Desorption | 60 | 60 | 1 |
| | 40 | 60 | 1 |

1 = Form 1 Compound 1 hydrogen chloride

### Example 24. Phosphoric Acid

Example 24A-Prior to acid addition, complete dissolution of Compound 1 (free base) was observed in all the solvent systems assessed. Following Salt Preparation Method 1 and using 1 mol. equiv. of phosphoric acid, solid materials were produced in all solvent systems except acetonitrile and 2-propanol:water (75:25% v/v) from which gum and oil were obtained, respectively. Crystalline Form 1 Compound 1 hydrogen phosphate was obtained from ethanol and THE samples. Damp and dried samples from ethyl acetate were poorly crystalline and could not be assigned any solid form. After 24 h storage at 40° C./75% RH, recrystallisation to novel crystalline Form 2 Compound 1 hydrogen phosphate was observed. The damp sample from MEK was found to be a novel crystalline Form 3 Compound 1 hydrogen phosphate. Conversion to crystalline Form 2 Compound 1 hydrogen phosphate was observed upon drying and after 48 h storage at 40° C./75% RH. Samples from acetonitrile and 2-propanol:water (75:25% v/v) were found to be amorphous by XRPD. After 24 h storage at 40° C./75% RH, recrystallisation to novel crystalline Form 4 Compound 1 hydrogen phosphate was observed for the sample from acetonitrile. The results are summarized in Table 65.

TABLE 65
Salt screen - Phosphoric acid, 1 equiv.

| | | XRPD Analysis | | |
|---|---|---|---|---|
| Initial Solvent System | AS | Damp | Dry | 40° C. 75% RH |
| Acetonitrile | tBME | A | A | 4 |
| Ethanol | Heptane | 1 | 1 | 1 |
| Ethyl acetate | Heptane | PC | PC | 2 |
| MEK | Heptane | 3 | 2 | 2 |
| THF | Heptane | 1 | 1 | 1 |
| IPA:H₂O (75:25% v/v) | tBME | A | A | A |

A = amorphous
PC = poorly crystalline
1 = Form 1 Compound 1 hydrogen phosphate
2 = Form 2 Compound 1 hydrogen phosphate
3 = Form 3 Compound 1 hydrogen phosphate
4 = Form 4 Compound 1 hydrogen phosphate

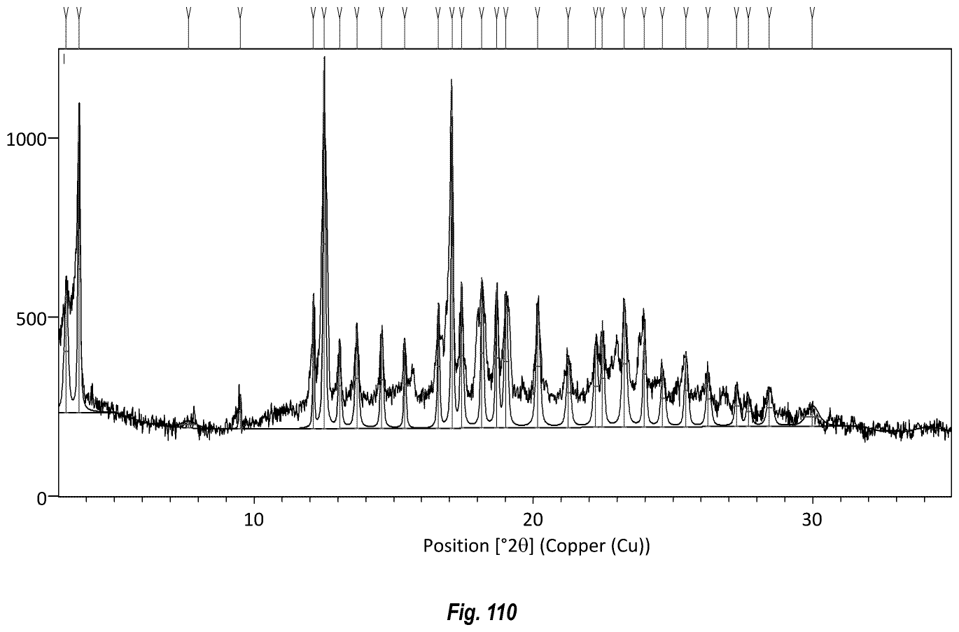

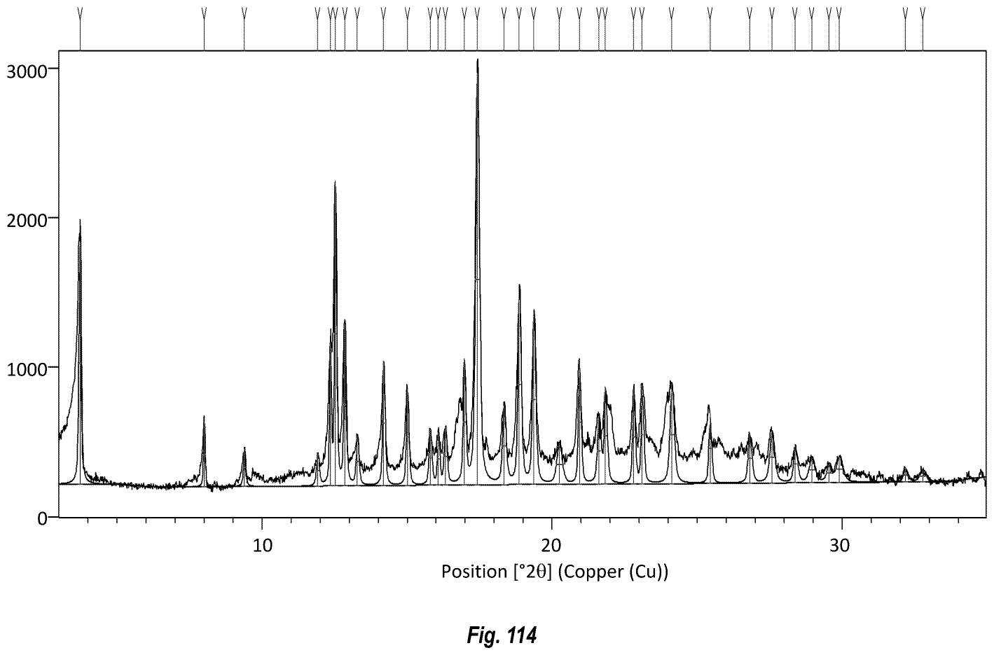

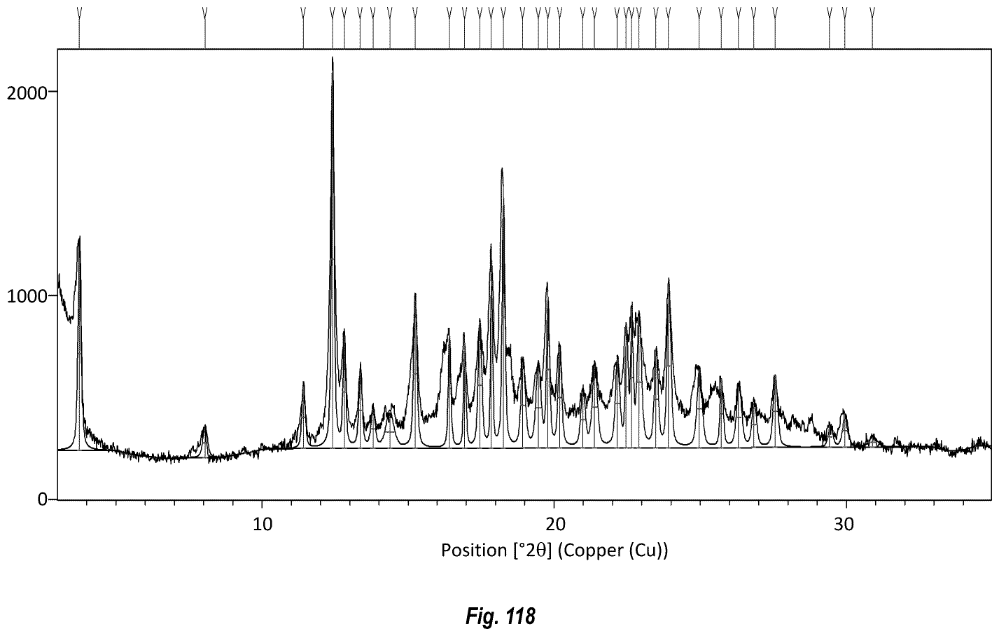

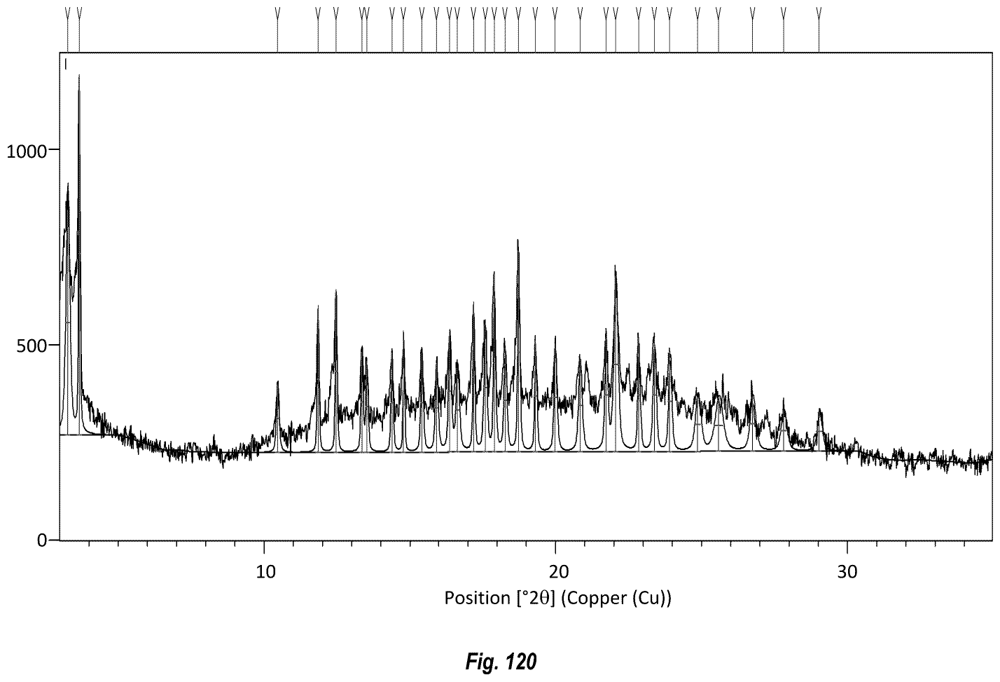

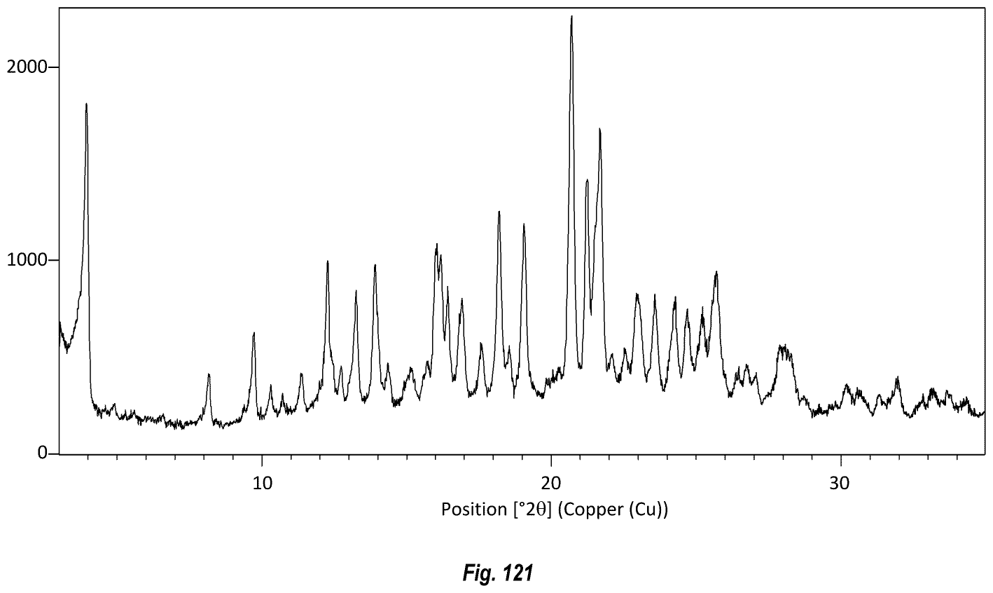

The XRPDs of crystalline Form 1 Compound 1 hydrogen phosphate are shown in FIGS. 109 and 110. The XRPDs of crystalline Form 2 Compound 1 hydrogen phosphate are shown in FIGS. 113 and 114. The XRPDs of crystalline Form 3 Compound 1 hydrogen phosphate are shown in FIGS. 117 and 118. The XRPDs of crystalline Form 4 Compound 1 hydrogen phosphate are shown in FIGS. 119 and 120. Crystalline Form 1 Compound 1 hydrogen phosphate and crystalline Form 2 Compound 1 hydrogen phosphate obtained from ethanol and MEK, respectively, were further analyzed as discussed below.

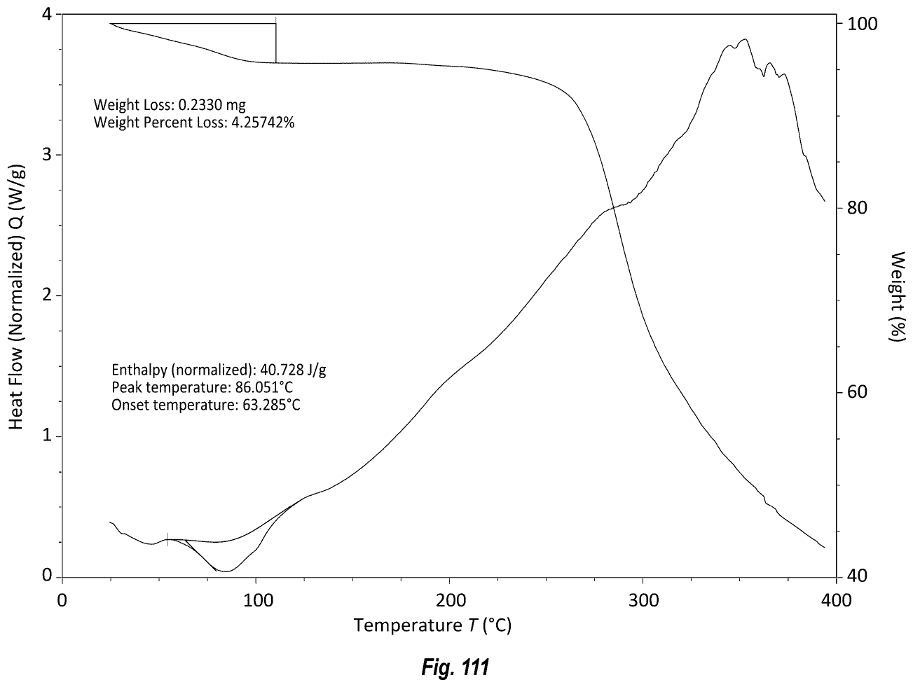

Crystalline Form 1 Compound 1 hydrogen phosphate—The TG/DSC of the dried sample from ethanol showed 4.3% mass loss between the outset and 110° C. A broad endothermic event was observed at peak onset 63° C. (peak at 86° C.). See FIG. 111.

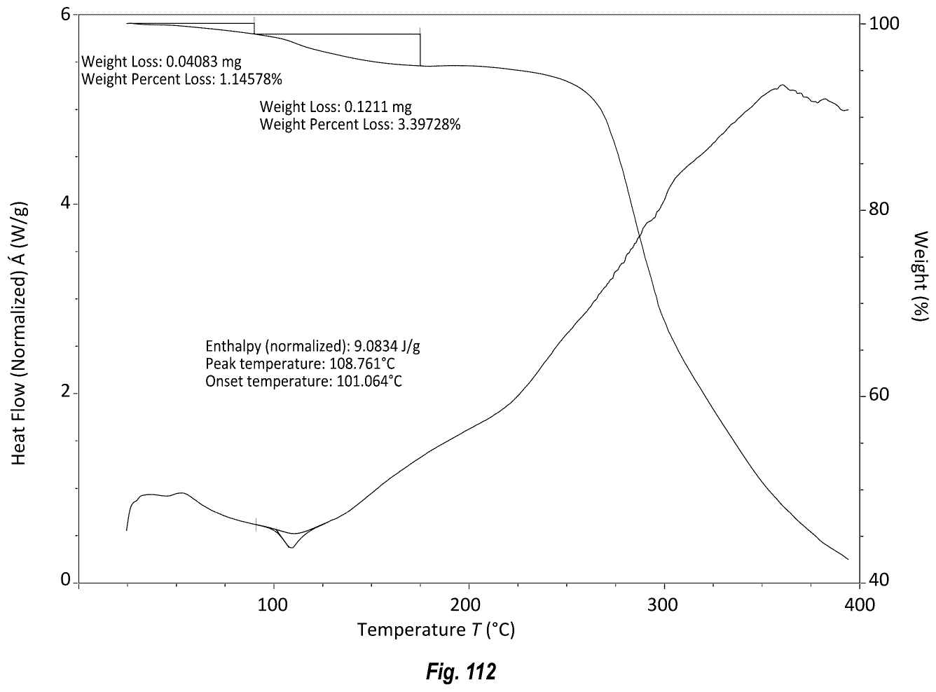

After 48 h storage at 40° C./75% RH, the sample from ethanol showed a 1.1% mass loss between the outset and 90° C., with a further loss of 3.4% (corresponding to 1.2 equiv. H2O) between 90-175° C. A small endothermic event was observed at peak onset 101° C. (peak at 109° C.). See FIG. 112.

The ¹H-NMR spectrum was consistent with the structure of the molecule, with peak shifting observed when compared with Compound 1 (an indication of salt formation). Peaks corresponding to 0.4% w/w heptane and 0.2% w/w ethanol were observed in the spectrum.

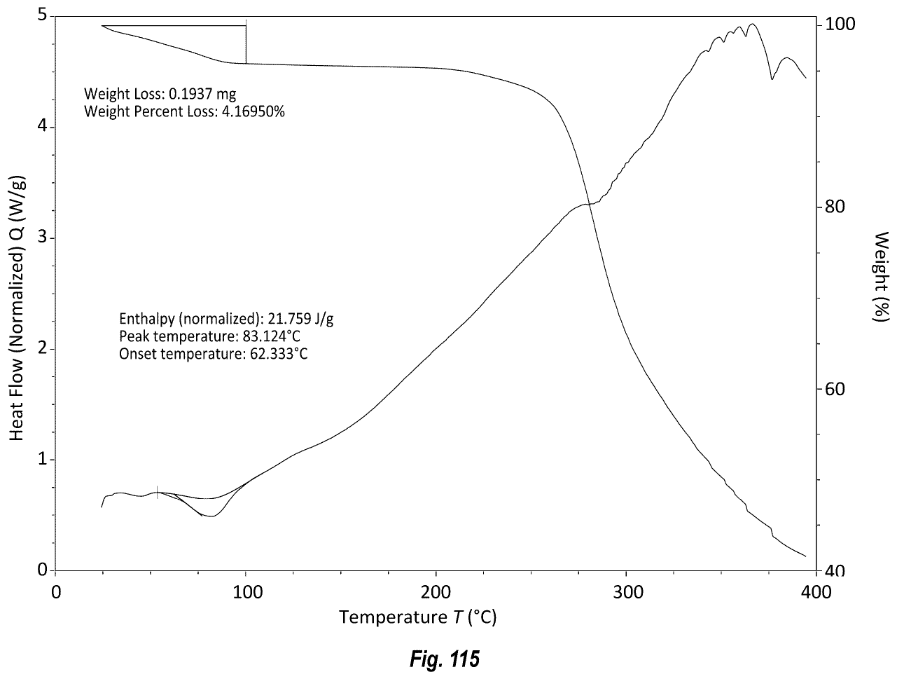

Crystalline Form 2 Compound 1 hydrogen phosphate—The TG/DSC of the dried sample from MEK showed 4.2% mass loss between the outset and 100° C. A broad endothermic event was observed at peak onset 62° C. (peak at 83° C.). See FIG. 115.

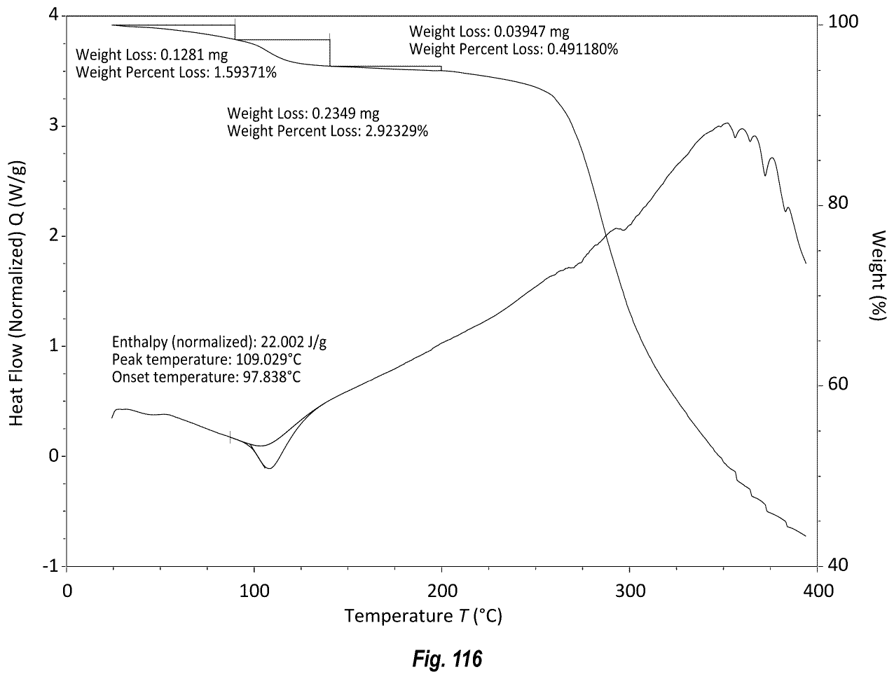

After 48 h storage at 40° C./75% RH, the sample from MEK showed a 1.6% mass loss between the outset and 90° C., a further loss of 2.9% (corresponding to 1 equiv. H₂O) between 90-140° C., and a small loss of 0.5% between 140-200° C. An endothermic event was observed at peak onset 98° C. (peak at 109° C.). See FIG. 116.

The ¹H-NMR spectrum was consistent with the structure of the molecule, with peak shifting observed when compared with Compound 1 (an indication of salt formation). Peaks corresponding to 1.0% w/w heptane and 0.1% w/w MEK were observed in the spectrum.

Example 24B—Prior to acid addition, complete dissolution of Compound 1 (free base) was observed in all the solvent systems assessed. Following Salt Preparation Method 1 and using 2 mol. equiv. of phosphoric acid, the isolated material was observed to be solid from all the solvent systems assessed, except 2-propanol:water (75:25% v/v) from which oil was obtained. Crystalline Form 5 Compound 1 hydrogen phosphate was obtained from acetonitrile. Upon storage at 40° C./75% RH, crystalline Form 5 Compound 1 hydrogen phosphate was retained but with additional peaks which corresponded to crystalline Form 6 Compound 1 hydrogen phosphate. Crystalline Form 6 Compound 1 hydrogen phosphate was also obtained from ethanol and ethyl acetate. The damp sample from THF was found to be crystalline Form 7 Compound 1 hydrogen phosphate, which upon drying under vacuum at ca. 40° C. was converted a crystalline Form 8 Compound 1 hydrogen phosphate. Upon storage at 40° C./75% RH, conversion of crystalline Form 8 Compound 1 hydrogen phosphate to crystalline Form 6 Compound 1 hydrogen phosphate was observed. Damp and dried samples from MEK were found to be poorly crystalline and could not be assigned any solid form. Upon storage at 40° C./75% RH, conversion of these samples from MEK to crystalline Form 6 Compound 1 hydrogen phosphate was observed. The samples obtained from 2-propanol:water (75:25% v/v) were found to be predominantly amorphous by XRPD. The results are summarized in Table 66.

TABLE 66
Salt screen - Phosphoric acid, 2 equiv.

| | | XRPD Analysis | | |
|---|---|---|---|---|
| Initial Solvent System | AS | Damp | Dry | 40° C. 75% RH |
| Acetonitrile | tBME | 5 | 5 | 5 + 6 |
| Ethanol | Heptane | 6 | 6 | 6 |
| Ethyl acetate | Heptane | 6 | 6 | 6* |
| MEK | Heptane | PC | PC | 6* |
| THF | Heptane | 7 | 8 | 6 |
| IPA:H₂O (75:25% v/v) | tBME | A* | A | A |

*Predominantly
**Material adhered the walls and bottom of the vials
A = amorphous
PC = poorly crystalline
5 = Form 5 Compound 1 hydrogen phosphate
6 = Form 6 Compound 1 hydrogen phosphate
7 = Form 7 Compound 1 hydrogen phosphate
8 = Form 8 Compound 1 hydrogen phosphate

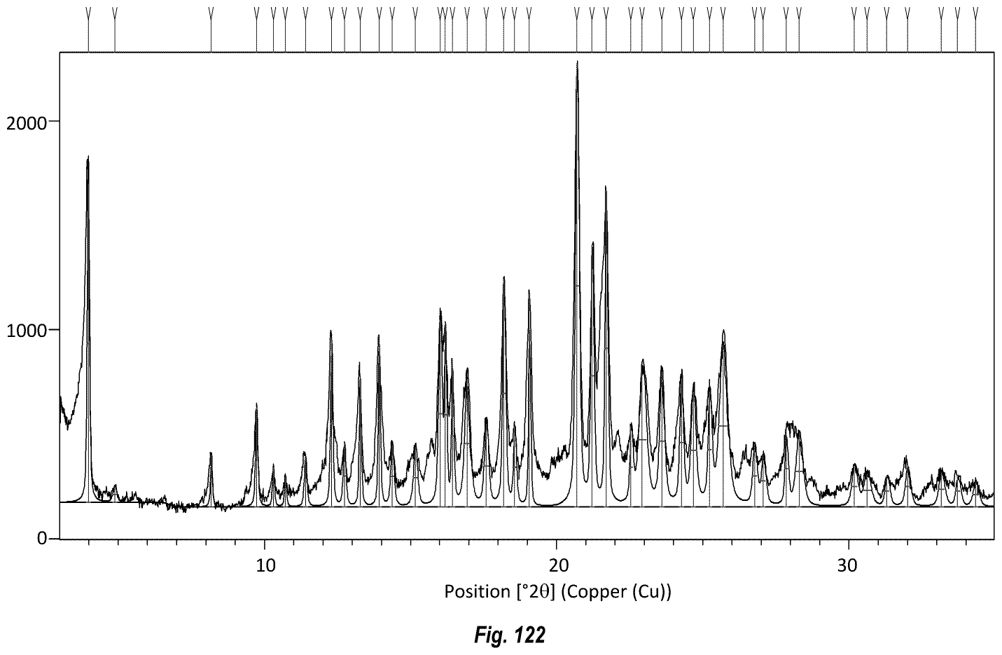

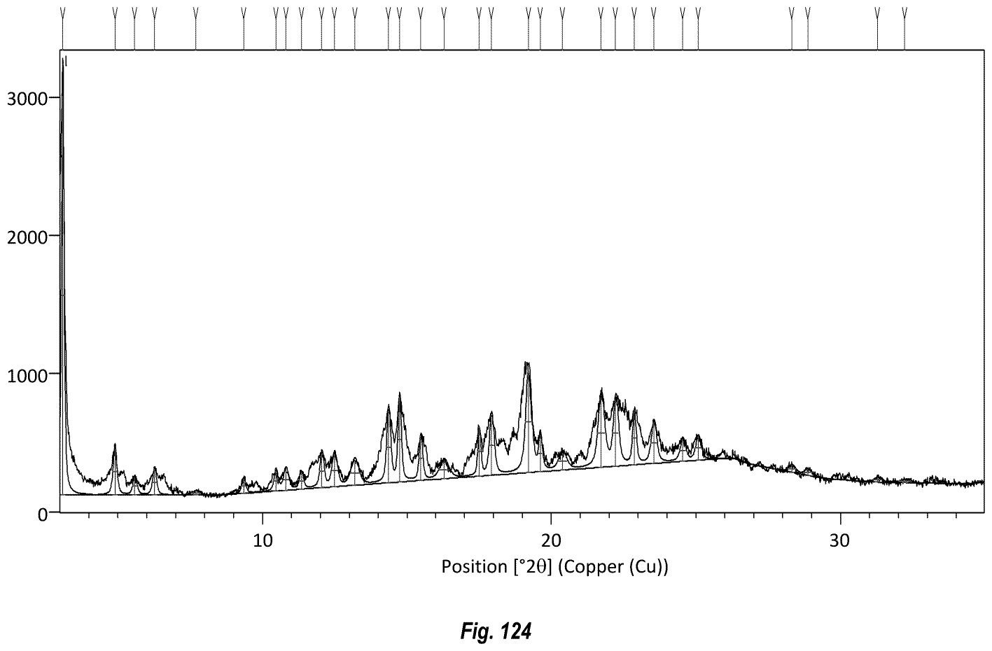

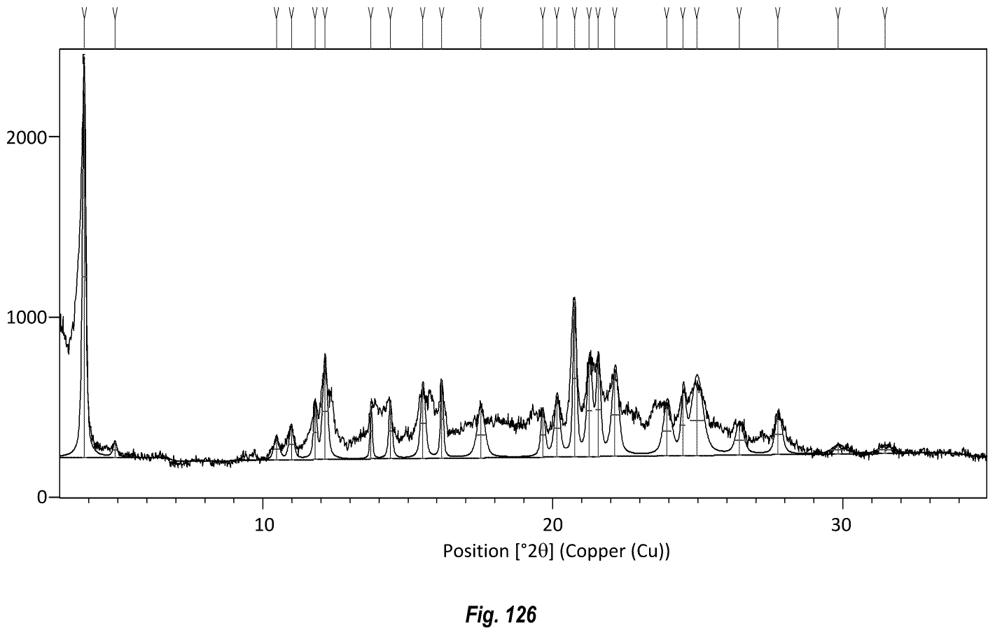

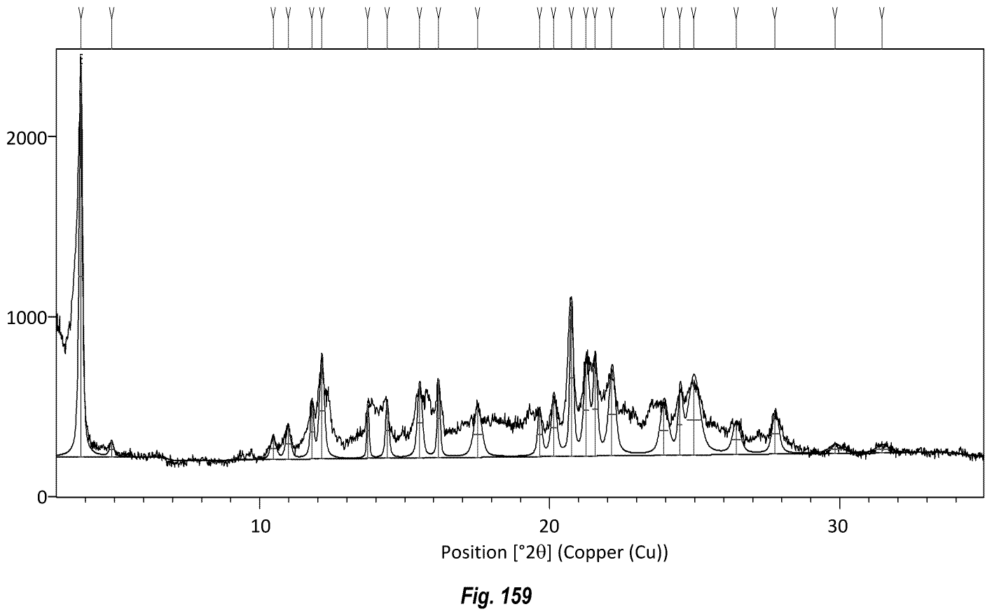

The XRPDs of crystalline Form 5 Compound 1 hydrogen phosphate are shown in FIGS. 121 and 122. The XRPDs of crystalline Form 6 Compound 1 hydrogen phosphate are shown in FIGS. 123 and 124. The XRPDs of crystalline Form 7 Compound 1 hydrogen phosphate are shown in FIGS. 158 and 159. The XRPDs of crystalline Form 8 Compound 1 hydrogen phosphate are shown in FIGS. 125 and 126. Crystalline Form 5 Compound 1 hydrogen phosphate (obtained from acetonitrile), crystalline Form 6 Compound 1 hydrogen phosphate (obtained from ethanol and THF), and crystalline Form 8 Compound 1 hydrogen phosphate (obtained from THF) were further analyzed as discussed below.

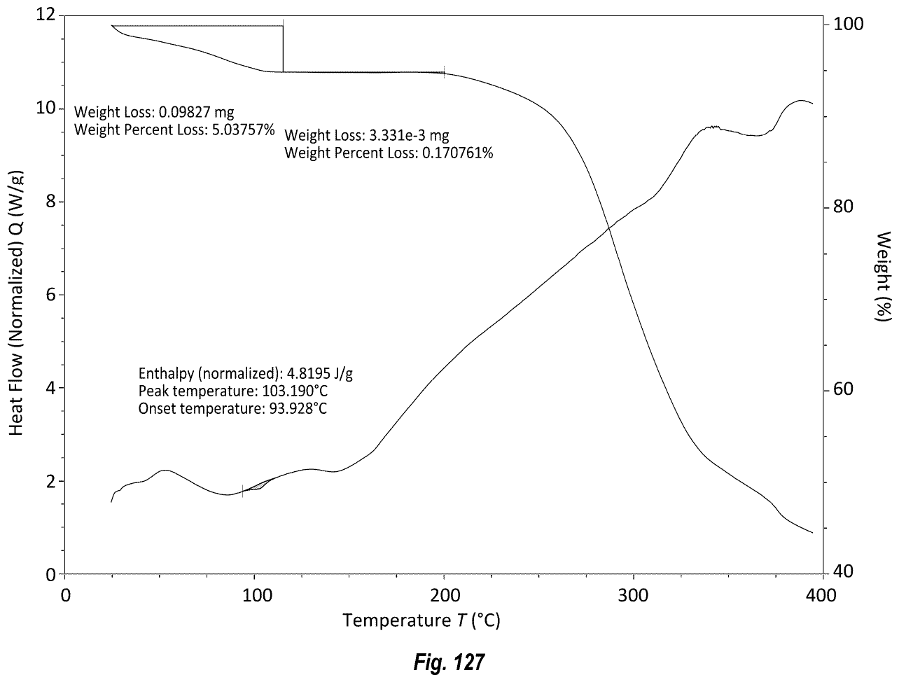

Crystalline Form 5 Compound 1 hydrogen phosphate—The TG/DSC of the dried sample from acetonitrile showed 5.0% mass loss between outset −115° C., followed by a further mass loss of 0.2% between 115-200° C. See FIG. 127. An endothermic event was observed at peak onset 94° C. (peak at 103° C.).

The ¹H-NMR spectrum was consistent with the structure of the molecule, with peak shifting observed when compared with the input Compound 1 (an indication of salt formation). Peaks corresponding to 1.6% w/w acetonitrile (overlapped) and <0.1% w/w tBME were observed in the spectrum.

Crystalline Form 6 Compound 1 hydrogen phosphate from ethanol—The TG/DSC of the dried sample from ethanol showed 2.9% mass loss between outset −160° C., followed by a further mass loss of 0.6% between 160-200° C. See FIG. 128. An endothermic event was observed at peak onset 169° C. (peak at 177° C.).

The ¹H-NMR spectrum was consistent with the structure of the molecule, with peak shifting observed when compared with Compound 1 (an indication of salt formation). Peaks corresponding to 1.6% w/w ethanol and 1.9% w/w heptane were observed in the spectrum.

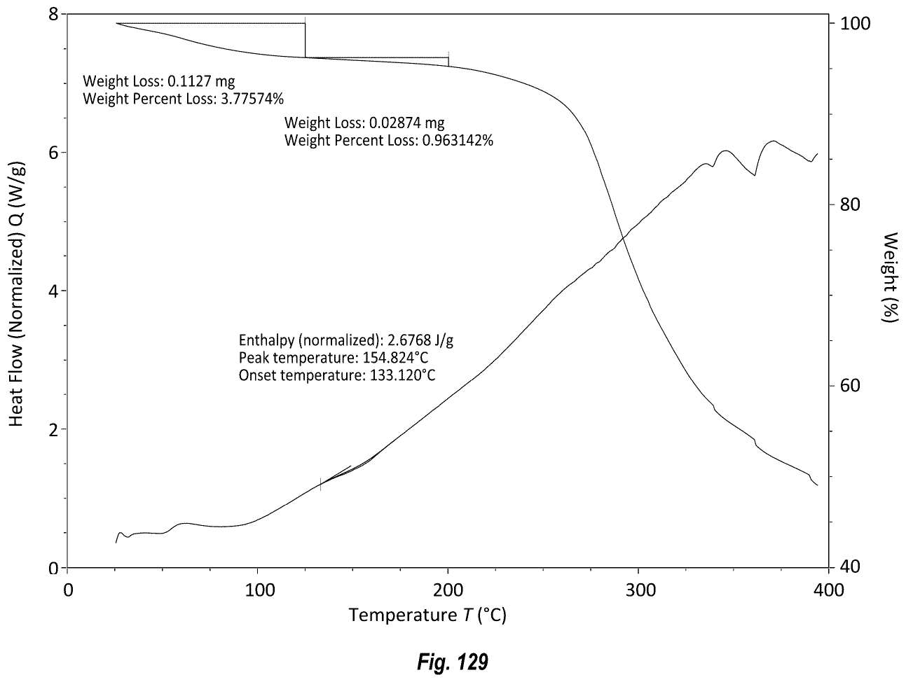

Crystalline Form 6 Compound 1 hydrogen phosphate from THF—The TG/DSC of the 40° C./75% RH sample from THE showed 3.8% mass loss between outset −125° C., followed by a further mass loss of 1.0% between 125-200° C. See FIG. 129. A shallow endothermic event was observed at peak onset 133° C. (peak at 155° C.).

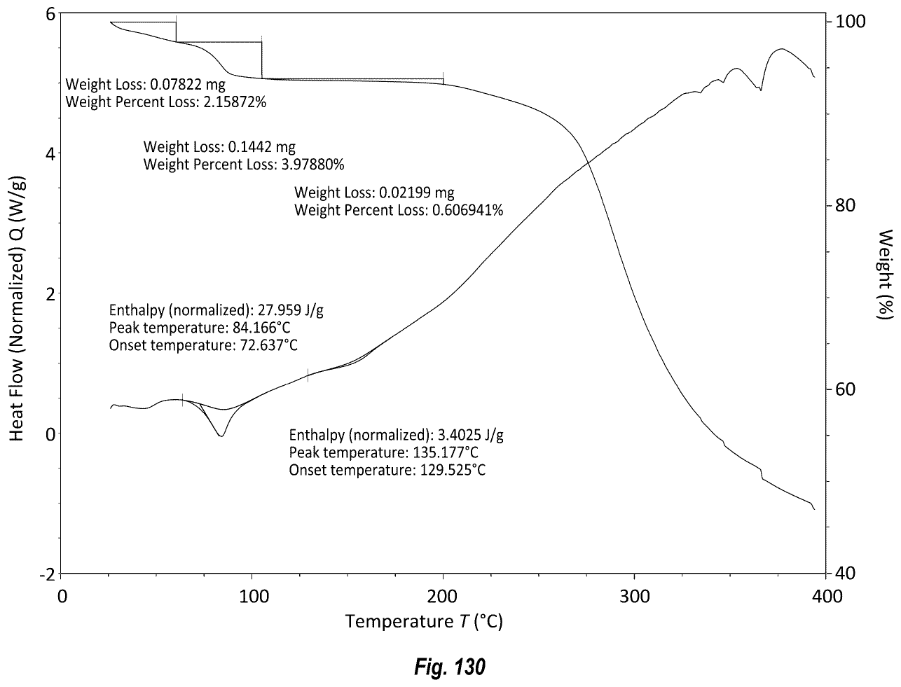

Crystalline Form 8 Compound 1 hydrogen phosphate—The TG/DSC of the dried sample from THE showed 2.2% mass loss between outset −60° C. This was followed by a further mass loss of 4.0% between 60-105° C., and 0.6% between 105-200° C. See FIG. 130. Endothermic events were observed at peak onsets 73° C. (peak at 84° C.) and 130° C. (peak at 135° C.).

The ¹H-NMR spectrum was consistent with the structure of the molecule, with peak shifting observed when compared with Compound 1 (an indication of salt formation). Peaks corresponding to 2.0% w/w THF (overlapped) and 1.0% w/w heptane were observed in the spectrum.

Example 24C—Crystalline Form 6 Compound 1 hydrogen phosphate was also prepared following the general procedure outlined in Salt Preparation Method 2. More specifically, the salt was prepared as follows. A known mass of Compound 1 (free base), 2.05 mol. equiv. of phosphoric acid, and 8 vol. of ethyl acetate were added to a 20 mL scintillation vial. The experiment was stirred at 5° C., and gum formation was observed. 6 vol. of anti-solvent (heptane) was added at 5° C., and slurry was observed after stirring at 5° C. for 24 h. The solids were isolated via vacuum filtration and dried under vacuum at ca. 40° C. for 24 h. The damp (sticky solid) and dried solids were subsampled and analyzed by XRPD. Due to poor crystallinity of the dried solids, these were stored at 40° C./75% RH for 24 h in order to improve the crystallinity. The final solid sample was characterized as discussed below.

Characterization of the prepared crystalline Form 6 Compound 1 hydrogen phosphate yielded the following results.

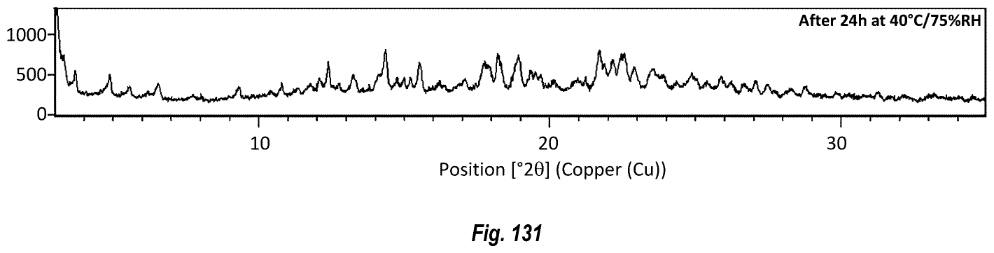

The XRPD of the final sample (after storage at 40° C./75% RH) is shown in FIG. 131.

PLM images showed slightly birefringent particles with no clear morphology.

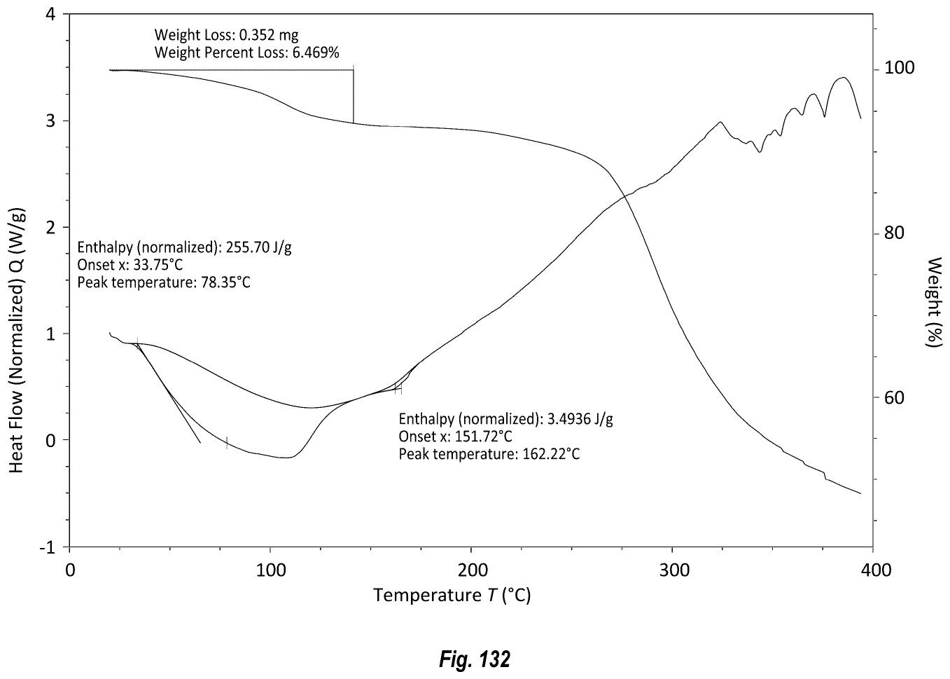

TG/DSC showed a 6.5% mass loss between 20° C.-140° C., which corresponded with an endothermic event at peak onset 34° C. (peak at 78° C.). This was likely due to water/solvent loss (theoretically, 6.5% w/w=3.1 mol. equiv. of H₂O). See FIG. 132. A small endothermic event was observed at peak onset 152° C. (peak at 162° C.). This was likely due to a melting event. The thermal events observed above 200° C. were likely due to degradation. DSC analysis showed two endothermic events at peak onsets 94° C. (peak at 96° C.) and 106° C. (peak at 113° C.). These were likely water loss. A third endothermic event was observed at peak onset 141° C. (peak at 163° C.), which was likely a melting event.

¹H-NMR spectrum was consistent with the received structure and showed peak shifts with respect to the Compound 1 material (indicating salt formation). No significant peaks corresponding to residual solvents were observed.

DVS analysis (carried out at 25° C.) showed a moisture uptake of ca. 3.5% between 40-80% RH during the first sorption cycle and an uptake of 3.2% was observed between 0-80% RH during the second sorption cycle, indicating moderate hygroscopicity. A steep uptake of 7.5% was observed between 80-90% RH. This was likely due to the sample deliquescing at high humidity conditions. Hysteresis of ca. 3% was observed between the sorption and desorption cycles. The post-DVS sample was observed to be gum-like material further indicating prior deliquescing.

DVS analysis (carried out at 40° C.) showed a moisture uptake of 6.4% between 40-80% RH during the first sorption cycle, and an uptake of 9.6% was observed between 0-80% RH during the second sorption cycle. This was an indication of moderate hygroscopicity. A further uptake of ca. 8.8% was observed between 80-90% RH, which was an indication of possible deliquescing. Hysteresis of ca. 2.5% was observed between the sorption and desorption cycles. The post-DVS sample was observed to be gum-like material further indicating prior deliquescing.

HPLC analysis indicated a solid purity of 99.25% area. HPLC-CAD indicated the presence of 2.75 mol. equiv. of phosphate.

VH-XRPD analysis carried out between 8-80% RH showed the crystalline Form 6 Compound 1 hydrogen phosphate being retained throughout. See Table 67.

TABLE 67
Results of VH-XRPD analysis for crystalline
Form 6 Compound 1 hydrogen phosphate

| | Rel. Humidity | Hold time | XRPD |
|---|---|---|---|
| Cycle | (% RH) | (min) | Results |
| Desorption | 40 | 60 | 6 |
| | 20 | 60 | 6 |
| | 10 | 60 | 6 |
| | 8 | 60 | 6 |
| Sorption | 20 | 60 | 6 |
| | 40 | 960 | 6 |
| | 60 | 60 | 6 |
| | 70 | 60 | 6 |
| | 80 | 120 | 6 |
| Desorption | 60 | 60 | 6 |
| | 40 | 120 | 6 |

6 = Form 6 Compound 1 hydrogen phosphate

VT-XRPD analysis carried out between 30-160° C. showed crystalline Form 6 Compound 1 hydrogen phosphate being retained between 30-50° C. Conversion to crystalline Form 9 Compound 1 hydrogen phosphate was observed between 80-150° C. The sample melted (amorphous diffractogram) from 160° C. See Table 68.

TABLE 68
Results of VT-XRPD analysis for crystalline
Form 6 Compound 1 hydrogen phosphate

| | Temp | Hold time | XRPD |
|---|---|---|---|
| Cycle | (° C.) | (min) | Results |
| Heating | 30 | 0 | 6 |
| | 50 | 30 | 6 |
| | 80 | 30 | 9 |
| | 100 | 30 | 9 |
| | 120 | 30 | 9 |
| Cooling | 30 | 10 | 6 (trace of 9) |
| Heating | 120 | 30 | 9 |
| | 130 | 30 | 9 |
| | 150 | 30 | 9 |
| | 160 | 30 | Melt |

6 = Form 6 Compound 1 hydrogen phosphate
9 = Form 9 Compound 1 hydrogen phosphate

### Example 25. p-Toluenesulfonic Acid

Example 25A—Prior to acid addition, complete dissolution of the Compound 1 (free base) was observed in all the solvent systems assessed except acetonitrile where partial dissolution was observed instead. Following Salt Preparation Method 1 and using 1 mol. equiv. of p-toluenesulfonic acid, the isolated materials from acetonitrile, ethyl acetate, MEK, and THF were observed to be gum-like. Glassy solids were obtained from ethanol and 2-propanol: water (75:25% v/v). All the solids analyzed from these experiments were found to be amorphous by XRPD as shown in Table 69.

TABLE 69
Salt screen - p-Toluenesulfonic acid, 1 equiv.

| | | XRPD Analysis | | |
|---|---|---|---|---|
| Initial Solvent System | AS | Damp | Dry | 40° C. 75% RH |
| Acetonitrile | tBME | A | A | A |
| Ethanol | Heptane | A (evap.) | | A |
| Ethyl acetate | Heptane | A | A | A |
| MEK | Heptane | A | A | A |
| THF | Heptane | A | A | A |

| | | | |
|---|---|---|---|
| IPA:H₂O (75:25% v/v) | tBME | A (evap.) | A |

A = amorphous
evap. = isolated via evaporation

Example 25B—Prior to acid addition, complete dissolution of Compound 1 (free base) was observed in all the solvent systems assessed. Following Salt Preparation Method 1 and using 2 mol. equiv. of p-toluenesulfonic acid, the isolated materials from ethanol, ethyl acetate, MEK, and THF were observed to be gum-like material. Sticky solid was obtained from acetonitrile. Solid material was obtained from 2-propanol:water (75:25% v/v) via evaporation. All the samples analyzed from these experiments were found to be amorphous by XRPD as shown in Table 70.

TABLE 70
Salt screen - p-Toluenesulfonic acid, 2 equiv.

| | | XRPD Analysis | | |
|---|---|---|---|---|
| Initial Solvent System | AS | Damp | Dry | 40° C. 75% RH |
| Acetonitrile | tBME | A | A | A |
| Ethanol | Heptane | A | A | A |
| Ethyl acetate | Heptane | A | A | A |
| MEK | Heptane | A | A | A |
| THF | Heptane | A | A | A |

| | | | |
|---|---|---|---|
| IPA:H₂O (75:25% v/v) | tBME | A (evap.) | A |

A = amorphous
evap. = isolated via evaporation

### Example 26. Methanesulfonic Acid

Example 26A—Prior to acid addition, complete dissolution of Compound 1 (free base) was observed in all the solvent systems assessed except acetonitrile where partial dissolution was observed instead. Following Salt Preparation Method 1 and using 1 mol. equiv. of methanesulfonic acid, the isolated materials from acetonitrile, ethyl acetate, MEK, and THF were observed to be gum-like. Glassy solids were obtained from ethanol and 2-propanol:water (75:25% v/v). All the solids analyzed from these experiments were found to be amorphous by XRPD as shown in Table 71.

TABLE 71
Salt screen - Methanesulfonic acid, 1 equiv.

| | | XRPD Analysis | | |
|---|---|---|---|---|
| Initial Solvent System | AS | Damp | Dry | 40° C. 75% RH |
| Acetonitrile | tBME | A | A | A |
| Ethanol | Heptane | A (evap.) | | A |
| Ethyl acetate | Heptane | A | A | A |
| MEK | Heptane | A | A | A |
| THF | Heptane | A | A | A |

| | | | |
|---|---|---|---|
| IPA:H₂O (75:25% v/v) | tBME | A (evap.) | A |

A = amorphous
evap. = isolated via evaporation

Example 26B—Prior to acid addition, complete dissolution of Compound 1 (free base) was observed in all the solvent systems assessed. Following Salt Preparation Method 1 and using 2 mol. equiv. of methanesulfonic acid, the isolated materials from acetonitrile, ethanol and MEK were observed to be gum-like material. Sticky solids were obtained from ethyl acetate and THF. Solid material was obtained from 2-propanol:water (75:25% v/v) via evaporation. All the samples analyzed from these experiments were found to be amorphous by XRPD as shown in Table 72.

TABLE 72
Salt screen - Methanesulfonic acid, 2 equiv.

| | | XRPD Analysis | | |
|---|---|---|---|---|
| Initial Solvent System | AS | Damp | Dry | 40° C. 75% RH |
| Acetonitrile | tBME | A | A | A |
| Ethanol | Heptane | A | A | A |
| Ethyl acetate | Heptane | A | A | A |
| MEK | Heptane | A | A | A |
| THF | Heptane | A | A | A |

| | | | |
|---|---|---|---|
| IPA:H₂O (75:25% v/v) | tBME | A (evap.) | A |

A = amorphous
evap. = isolated via evaporation

### Example 27. Benzenesulfonic Acid

Example 27A—Prior to acid addition, complete dissolution of Compound 1 (free base) was observed in all the solvent systems assessed. Following Salt Preparation Method 1 and using 1 mol. equiv. of benzenesulfonic acid, the isolated materials were all observed to be gum-like material except 2-propanol:water (75:25% v/v) where sticky solid was obtained. All the samples analyzed from these experiments were found to be amorphous by XRPD as shown in Table 73.

TABLE 73
Salt screen - Benzenesulfonic acid, 1 equiv.

| | | XRPD Analysis | | |
|---|---|---|---|---|
| Initial Solvent System | AS | Damp | Dry | 40° C. 75% RH |
| Acetonitrile | tBME | A | A | A |
| Ethanol | Heptane | A | A | A |
| Ethyl acetate | Heptane | A | A | A |
| MEK | Heptane | A | A | A |
| THF | Heptane | A | A | A |

| | | | |
|---|---|---|---|
| IPA:H₂O (75:25% v/v) | tBME | A (evap.) | A |

A = amorphous
evap. = isolated via evaporation

Example 27B—Prior to acid addition, complete dissolution of Compound 1 (free base) was observed in all the solvent systems assessed. Following Salt Preparation Method 1 and using 2 mol. equiv. of benzenesulfonic acid, the isolated materials from acetonitrile, ethanol and MEK were observed to be gum-like material. Sticky solids were obtained from ethyl acetate and THF. Oil was obtained from 2-propanol:water (75:25% v/v). All the samples analyzed from these experiments were found to be amorphous by XRPD as shown in Table 74.

TABLE 74
Salt screen - Benzenesulfonic acid, 2 equiv.

| | | XRPD Analysis | | |
|---|---|---|---|---|
| Initial Solvent System | AS | Damp | Dry | 40° C. 75% RH |
| Acetonitrile | tBME | A | A | A |
| Ethanol | Heptane | A | A | A |
| Ethyl acetate | Heptane | A | A | A |
| MEK | Heptane | A | A | A |
| THF | Heptane | A | A | A |
| IPA:H₂O (75:25) | tBME | A | A | A |

A = amorphous

### Example 28. L-Tartaric Acid

Prior to acid addition, complete dissolution of Compound 1 (free base) was observed in all the solvent systems assessed. Following Salt Preparation Method 1 and using 1 mol. equiv. of L-tartaric acid, the isolated materials from acetonitrile and ethanol were observed to be sticky solids. Isolated materials from ethyl acetate, MEK, and THF were observed to be solids. Isolated material from 2-propanol:water (75:25% v/v) was observed to be oil. All the samples analyzed from these experiments were found to be amorphous by XRPD as shown in Table 75. The damp and dried samples obtained from ethyl acetate were found to be consistent with the counterion (L-tartaric acid) with reduced crystallinity.

TABLE 75
Salt screen - L-Tartaric acid, 1 equiv.

| | | XRPD Analysis | | |
|---|---|---|---|---|
| Initial Solvent System | AS | Damp | Dry | 40° C. 75% RH |
| Acetonitrile | tBME | A | A | A |
| Ethanol | Heptane | A | A | A |
| Ethyl acetate | Heptane | PC, CI | PC, CI | A |
| MEK | Heptane | A | A | A |
| THF | Heptane | A | A | A |
| IPA:H₂O (75:25% v/v) | tBME | A | A | A |

A = amorphous
PC = poorly crystalline
CI = counterion
evap. = isolated via evaporation

### Example 29. Fumaric Acid

Example 29A—Prior to acid addition, complete dissolution of Compound 1 (free base) was observed in all the solvent systems assessed. Following Salt Preparation Method 1 and using 1 mol. equiv. of fumaric acid, the isolated materials from acetonitrile and ethyl acetate were observed to be solids. Isolated materials from ethanol and 2-propanol:water (75:25% v/v) were observed to be oil. Isolated materials from MEK and THF were observed to be sticky solids. Crystalline Form 1 Compound 1 fumarate was obtained from acetonitrile. After 24 h storage at 40° C./75% RH, crystalline Form 1 Compound 1 fumarate was retained but the XRPD showed additional peaks corresponding to crystalline Form 3 Compound 1 fumarate. The poorly crystalline Form 2 Compound 1 fumarate was obtained from ethyl acetate. After 24 h storage at 40° C./75% RH, conversion from crystalline Form 2 Compound 1 fumarate to crystalline Form 3 Compound 1 fumarate was observed. Samples obtained from ethanol, MEK, THF, and 2-propanol: water (75:25% v/v) were found to be amorphous by XRPD. After 24 h storage at 40° C./75% RH, these amorphous samples showed conversion to crystalline Form 3 Compound 1 fumarate except that from 2-propanol: water (75:25% v/v) which remained amorphous. The results are summarized in Table 76.

TABLE 76
Salt screen - Fumaric acid, 1 equiv.

| | | XRPD Analysis | | |
|---|---|---|---|---|
| Initial Solvent System | AS | Damp | Dry | 40° C. 75% RH |
| Acetonitrile | tBME | 1 | 1 | 1 + 3 |
| Ethanol | Heptane | A | A | 3 |
| Ethyl acetate | Heptane | 2, PC | 2, PC | 3 |
| MEK | Heptane | A | A | 3 |
| THF | Heptane | A | A | 3 |
| IPA:H₂O (75:25% v/v) | tBME | A | A | A |

A = amorphous
PC = poorly crystalline
1 = Form 1 Compound 1 fumarate
2 = Form 2 Compound 1 fumarate
3 = Form 3 Compound 1 fumarate

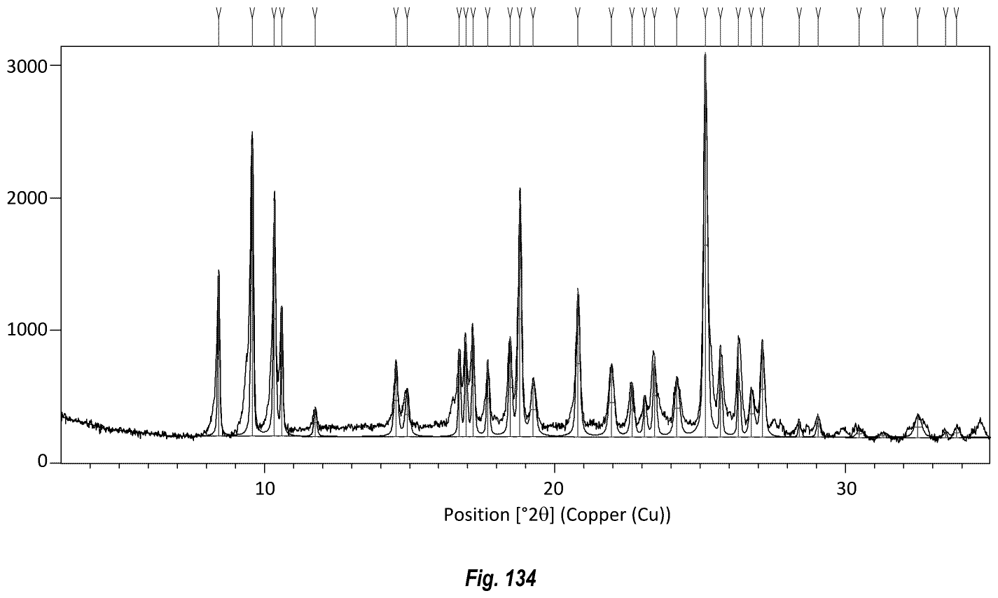

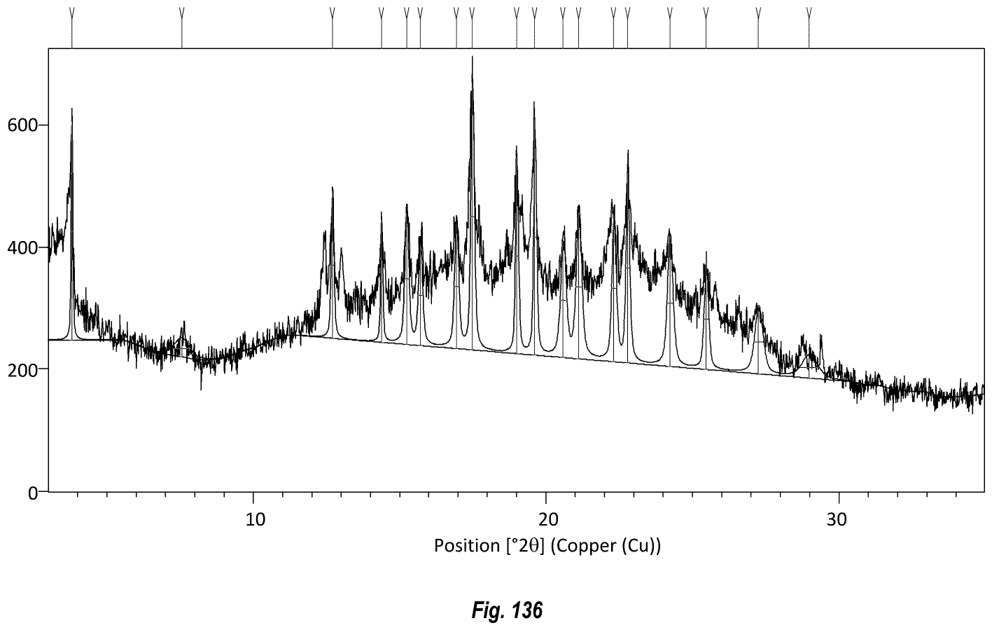

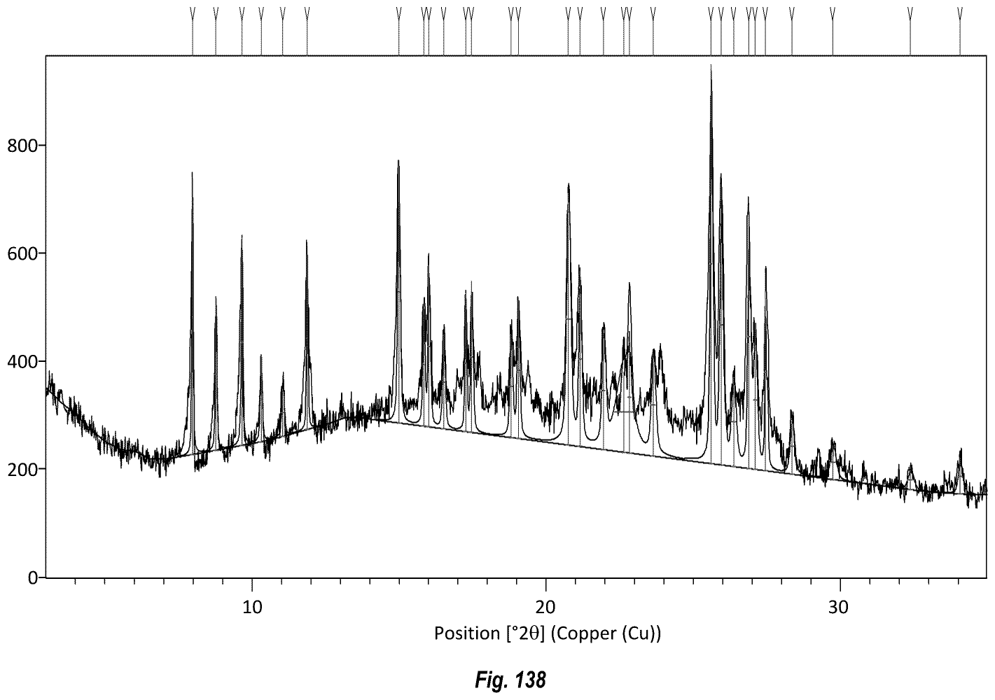

The XRPDs of crystalline Form 1 Compound 1 fumarate are shown in FIGS. 133 and 134. The XRPDs of crystalline Form 2 Compound 1 fumarate are shown in FIGS. 135 and 136. The XRPDs of crystalline Form 3 Compound 1 fumarate are shown in FIGS. 137 and 138. Crystalline Form 1 Compound 1 fumarate (from acetonitrile), crystalline Form 2 Compound 1 fumarate (from ethyl acetate), and crystalline Form 3 Compound 1 fumarate (from MEK) were further analyzed, as discussed below.

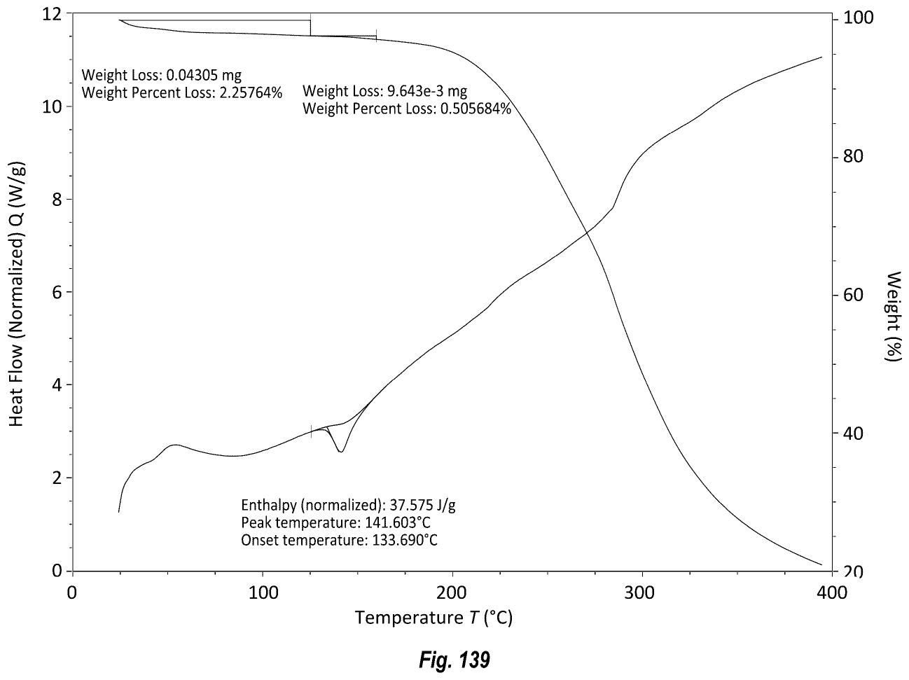

Crystalline Form 1 Compound 1 fumarate—The TG/DSC of the dried sample from acetonitrile showed 2.3% mass loss between outset −125° C. An endothermic event was observed at peak onset 134° C. (peak at 142° C.), which was concomitant with a further mass loss of 0.5%. See FIG. 139.

The ¹H-NMR spectrum was consistent with the structure of the molecule, with peak shifting observed when compared with Compound 1 (an indication of salt formation). A peak corresponding to 1.5 eq. of the counterion (fumaric acid) was observed at δ 6.7 ppm. Peaks corresponding to 0.3% w/w acetonitrile and 0.4% w/w tBME were observed in the spectrum.

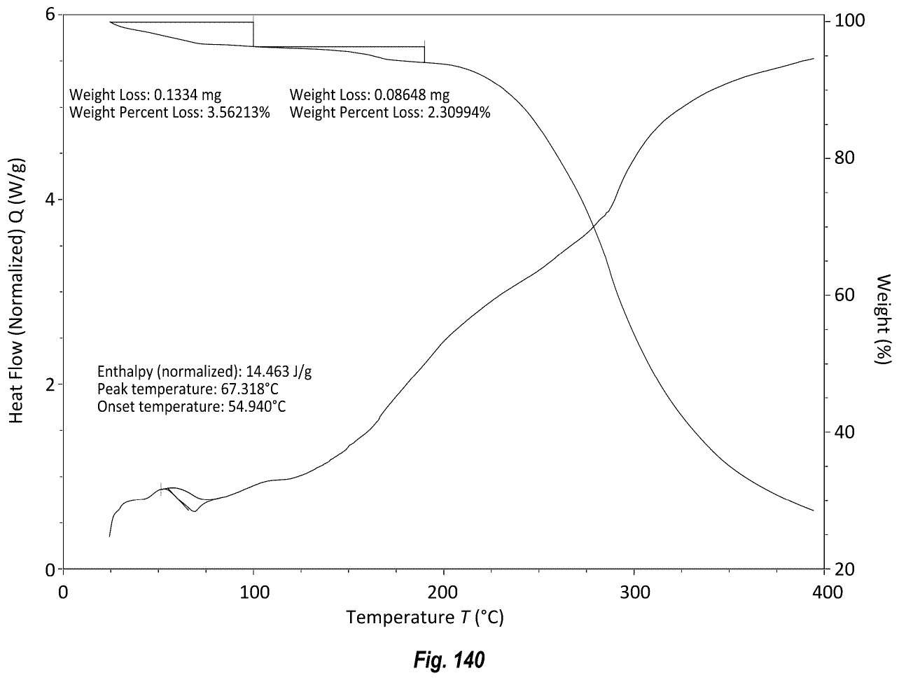

Crystalline Form 2 Compound 1 fumarate—The TG/DSC of the dried sample from ethyl acetate showed 3.6% mass loss between outset-100° C., followed a further mass loss of 2.3% between 100-190° C. (theoretically 1 equiv. of water). See FIG. 140. An endothermic event was observed at peak onset 55° C. (peak at 67° C.).

The ¹H-NMR spectrum was consistent with the structure of the molecule, with peak shifting observed when compared with Compound 1 (an indication of salt formation). A peak corresponding to 1.0 equiv. of the counterion (fumaric acid) was observed at δ 6.7 ppm. Peaks corresponding to 1.2% w/w ethyl acetate and 1.9% w/w heptane were observed in the spectrum.

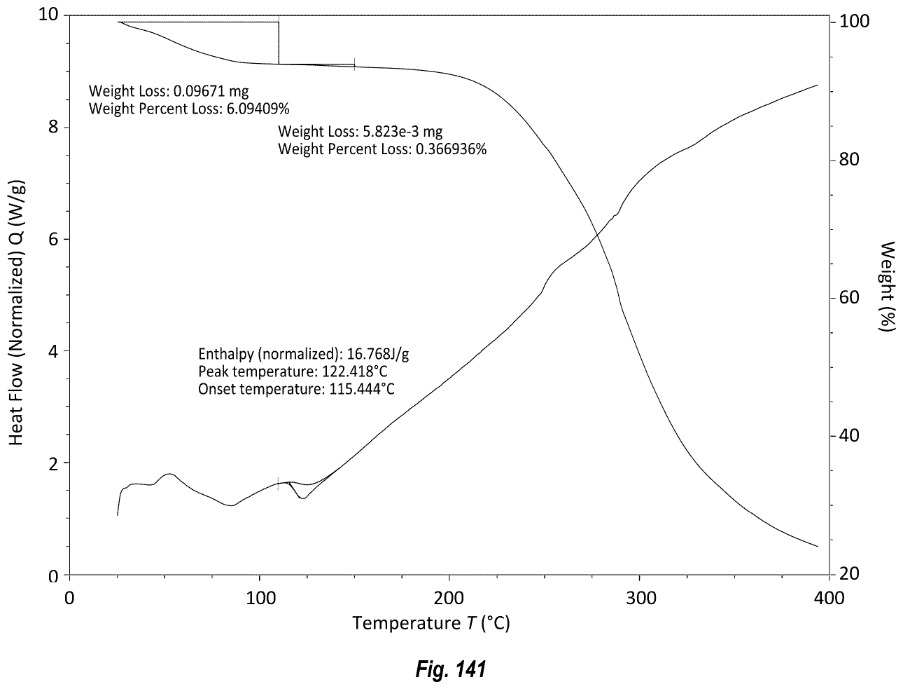

Crystalline Form 3 Compound 1 fumarate—The TG/DSC of the 40° C./75% RH sample from MEK showed 6.1% mass loss from outset −110° C. (2.7 equiv. of water, assuming a mono-salt). See FIG. 141. A further mass loss of 0.4% was observed between 110-150° C., which corresponded with an endothermic event at peak onset 115° C. (peak at 122° C.).

Example 29B—A mixture of crystalline Form 2 Compound 1 fumarate and crystalline Form 3 Compound 1 fumarate was prepared following the general procedure outlined in Salt Preparation Method 2. More specifically, the salt was prepared as follows. A known mass of Compound 1 (free base) and 4 vol. of ethyl acetate were added to a 20 mL scintillation vial. 1.05 mol. equiv. of fumaric acid was added to the vial as a slurry in 4 vol. of ethyl acetate. The sample was stirred at 40° C. for ca. 1 h, and then was temperature cycled between 40° C. and 5° C. with 0.1° C./min ramp and 1 h hold between each step. After ca. 48 h of cycling, gum formation was observed in the vial. Up to 16 vol. of anti-solvent (heptane) was then added at 5° C. to facilitate precipitation. The sample (now a mixture of gum and slurry) was further stirred at 5° C. for 4 days. The solids were isolated via vacuum filtration and dried under vacuum at ca. 40° C. for 24 h. The damp and dried solids were subsampled and analyzed by XRPD. XRPD results of the dried solids showed undesired solid form. The dried solids were then stored at 40° C./75% RH to facilitate solid form conversion to the desired form. After 26 days of storage, the residual solid sample (still a mixture of forms) was characterized as discussed below.

The sample obtained from the experiment showed a mixture of crystalline Form 2 Compound 1 fumarate and crystalline Form 3 Compound 1 fumarate after 26 days of 40° C./75% RH storage. The material was characterized as a mixture of solid forms.

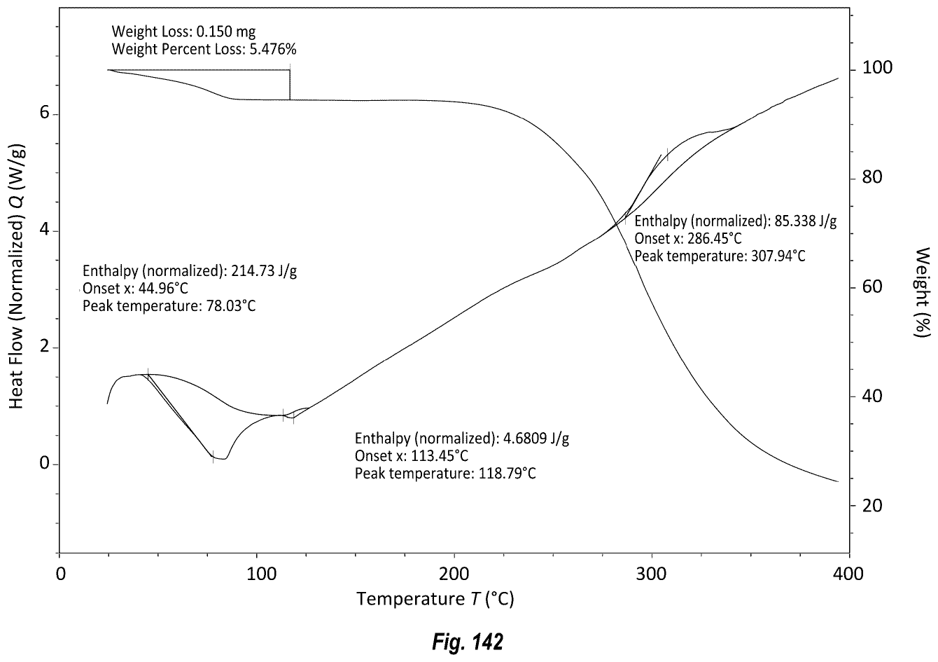

TG/DSC analysis showed a mass loss of ca. 5.5% between 20° C.-140° C., which corresponded with an endothermic event at peak onset 45° C. (peak at 78° C.). These are likely due to solvent/water loss (theoretically, 5.5%=2.4 mol. equiv. of H₂O). See FIG. 142. A small endothermic event was observed at peak onset 113° C. (peak at 119° C.). This is likely a melting event. The events observed above 200° C. are likely due to degradation.

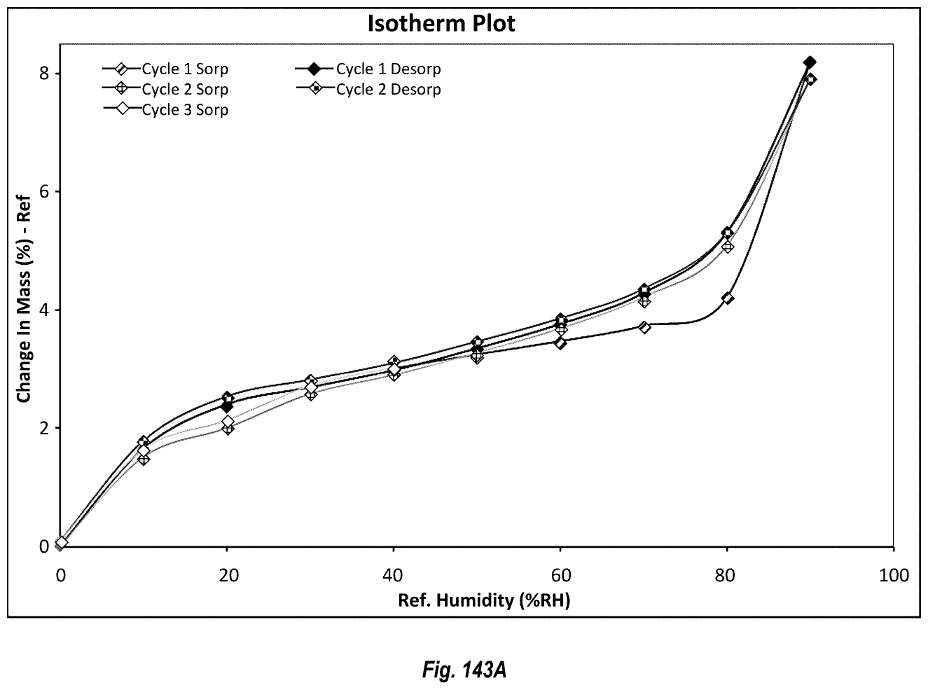

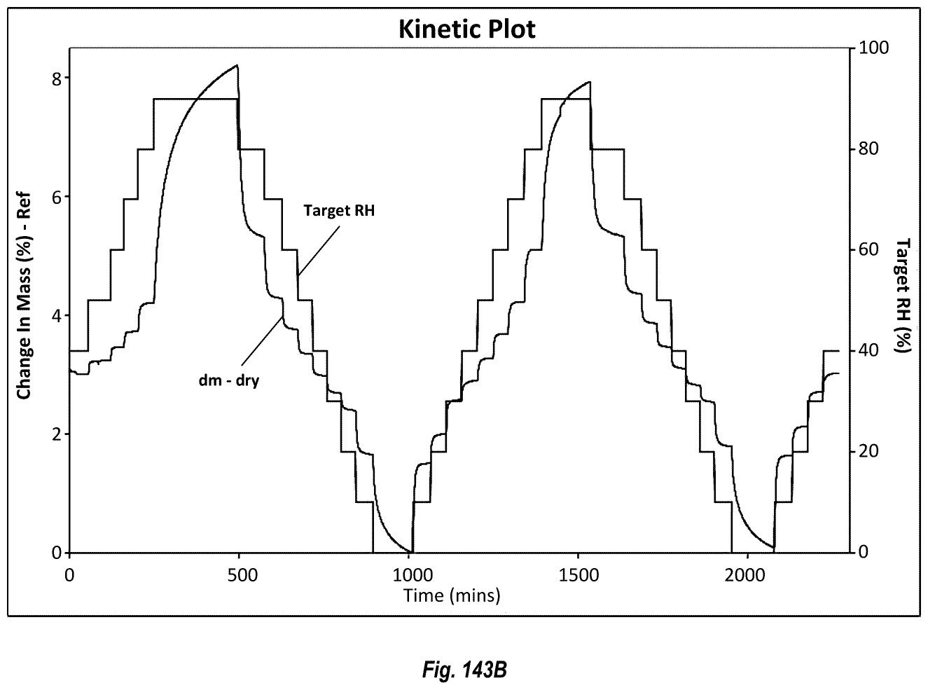

DVS analysis showed a moisture uptake of ca. 1.6% between 0-10% RH during the sorption cycles. A further uptake of ca. 3.5% was observed between 10-80% RH. See FIGS. 143A and 143B. A steep uptake of 4.0% was observed between 80-90% RH. A small hysteresis of ca. 0.4% was observed between the sorption and desorption cycles at 20% RH. The moisture uptake of the initial and final samples (at 40% RH) was ca. 3%. The post-DVS sample was found to be consistent with the input material (crystalline Form 2 Compound 1 fumarate and crystalline Form 3 Compound 1 fumarate) by XRPD.

### Example 30. Citric Acid

Prior to acid addition, complete dissolution of Compound 1 (free base) was observed in all the solvent systems assessed. Following Salt Preparation Method 1 and using 1 mol. equiv. of citric acid, the isolated materials from ethyl acetate, MEK, and THF were observed to be solids and isolated material from 2-propanol:water (75:25% v/v) was observed to be oil. All the samples analyzed were found to be amorphous by XRPD. The damp and dried samples obtained from ethyl acetate were found to be consistent with the counterion (citric acid) with reduced crystallinity. The results are summarized in Table 77.

TABLE 77
Salt screen - Citric acid, 1 equiv.

| | | XRPD Analysis | | |
|---|---|---|---|---|
| Initial Solvent System | AS | Damp | Dry | 40° C. 75% RH |
| Acetonitrile | tBME | A | A | A |
| Ethanol | Heptane | A | A | A |
| Ethyl acetate | Heptane | PC, CI | PC, CI | A |
| MEK | Heptane | A | A | A |
| THF | Heptane | A | A | A |
| IPA:H₂O (75:25% v/v) | tBME | A | A | A |

A = amorphous
PC = poorly crystalline
CI = Counterion

### Example 31. Maleic Acid

Prior to acid addition, complete dissolution of Compound 1 (free base) was observed in all the solvent systems assessed. Following Salt Preparation Method 1 and using 1 mol. equiv. of maleic acid, the isolated materials were all observed to be gum-like material except 2-propanol: water (75:25% v/v) where sticky solid was obtained. All the samples analyzed were found to be amorphous by XRPD as summarized in Table 78.

TABLE 78
Salt screen - Maleic acid, 1 equiv.

| | | XRPD Analysis | | |
|---|---|---|---|---|
| Initial Solvent System | AS | Damp | Dry | 40° C. 75% RH |
| Acetonitrile | tBME | A | A | A |
| Ethanol | Heptane | A | A | A |
| Ethyl acetate | Heptane | A | A | A |
| MEK | Heptane | A | A | A |
| THF | Heptane | A | A | A |

| | | | |
|---|---|---|---|
| IPA:H₂O (75:25% v/v) | tBME | A (evap.) | A |

A = amorphous
evap. = Isolated via evaporation

### Example 32. L-Malic Acid

Prior to acid addition, complete dissolution of Compound 1 (free base) was observed in all the solvent systems assessed. Following Salt Preparation Method 1 and using 1 mol. equiv. of L-malic acid, the isolated materials were all gum-like, except for ethyl acetate and MEK where solids were observed. A mixture of crystalline Form 1 Compound 1 L-malate and crystalline Form 2 Compound 1 L-malate was obtained from MEK. After 24 h storage at 40° C./75% RH, crystalline Form 1 Compound 1 L-malate was obtained (peaks associated with crystalline Form 2 Compound 1 L-malate were absent). All the other solids analyzed were found to be amorphous by XRPD. The results are summarized in Table 79.

TABLE 79
Salt screen - L-Malic acid, 1 equiv.

| | | XRPD Analysis | | |
|---|---|---|---|---|
| Initial Solvent System | AS | Damp | Dry | 40° C.75% RH |
| Acetonitrile | tBME | A | A | A |
| Ethanol | Heptane | * A | * A | A |
| Ethyl acetate | Heptane | A | A | A |
| MEK | Heptane | 1 + 2 | 1 + 2 | 1 |
| THE | Heptane | A | A | A |
| IPA:H₂O (75:25% v/v) | tBME | A | A | A |

A = amorphous
* = predominantly
1 = Form 1 Compound 1 L-malate
2 = Form 2 Compound 1 L-malate

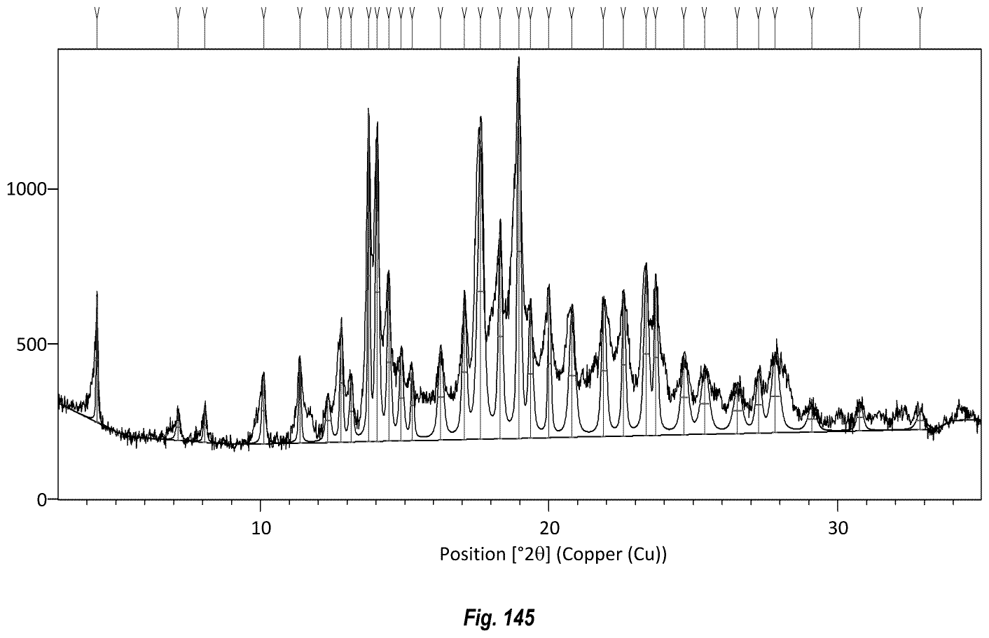

The XRPDs of crystalline Form 1 Compound 1 L-malate are shown in FIGS. 144 and 145. Crystalline Form 1 Compound 1 L-malate and a mixture of crystalline Form 1 Compound 1 L-malate and crystalline Form 2 Compound 1 L-malate obtained from MEK were further analyzed as discussed below.

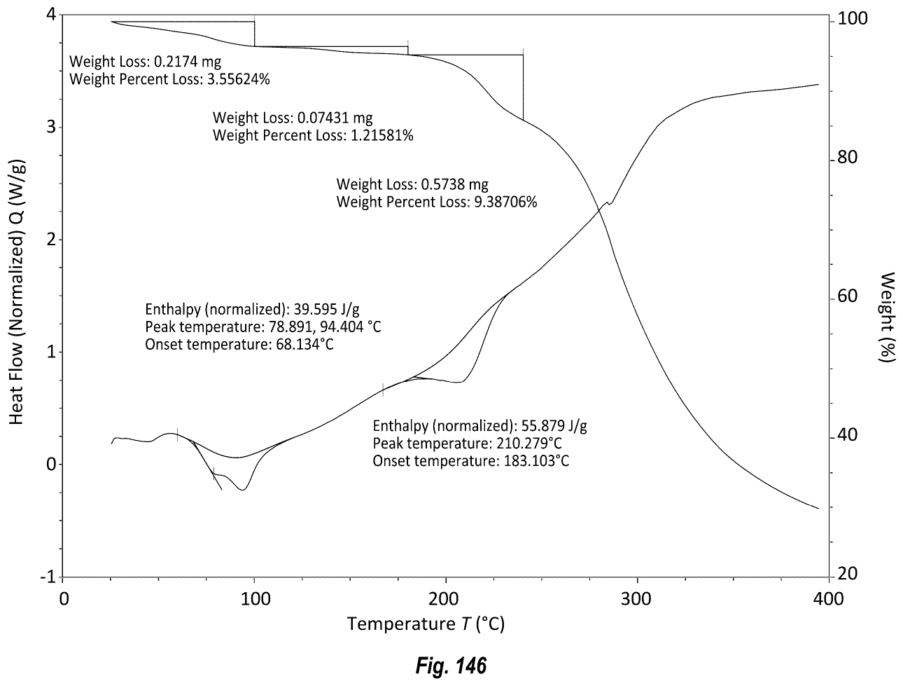

Crystalline Form 1 Compound 1 L-malate—The TG/DSC of the 40° C./75% RH sample from MEK showed 3.6% mass loss between the outset −100° C., followed by a further mass loss of 1.2% between 100-180° C., and 9.4% between 180-240° C. (theoretically, 17.5%=1 equiv. of malic acid; 9.6%=0.5 equiv.). See FIG. 146. Endothermic events were observed at peak onset 68° C. (peak at 79° C.) and peak onset 183° C. (peak at 210° C.).

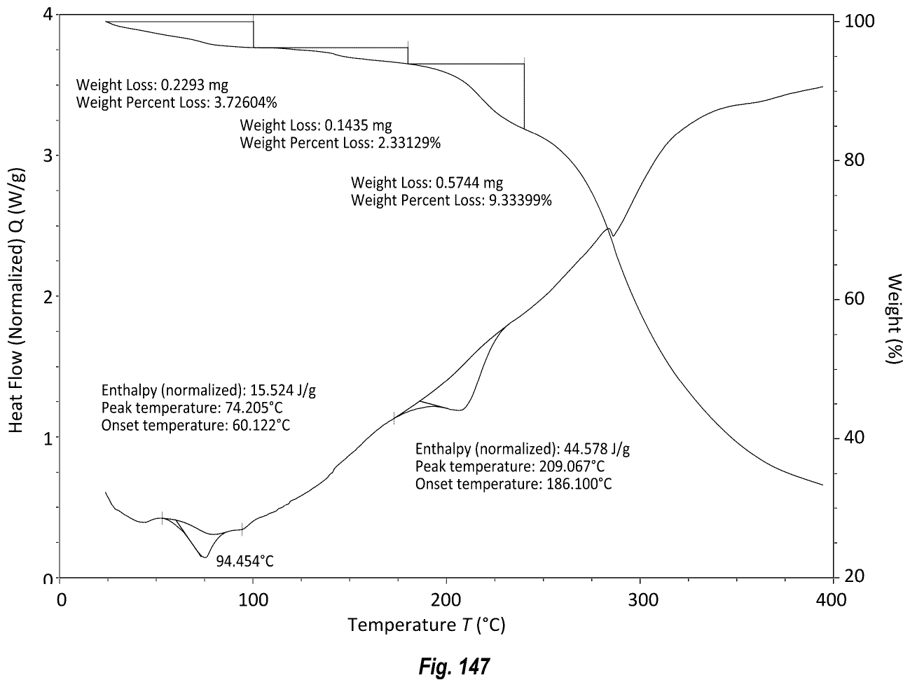

Mixture of Crystalline Form 1 Compound 1 L-malate and Crystalline Form 2 Compound 1 L-malate—The TG/DSC of the dried sample from MEK showed 3.7% mass loss between outset-100° C., followed by a further mass loss of 2.3% between 100-180° C. and 9.3% between 180-240° C. (theoretically, 17.5%=1 equiv. of malic acid; 9.6%=0.5 equiv.). See FIG. 147. Endothermic events were observed at peak onset 60° C. (peak at 74° C.) and peak onset 186° C. (peak at 209° C.).

The ¹H-NMR spectrum was consistent with the structure of the molecule, with peak shifting observed when compared with Compound 1 (an indication of salt formation). Peaks corresponding to 1.0 equiv. of the counterion (L-malic acid) were observed at δ 2.5, 2.8, and 4.3 ppm. Peaks corresponding to 1.4% w/w MEK and 1.6% w/w heptane were observed in the spectrum.

### Example 33. Benzoic Acid

Prior to acid addition, complete dissolution of Compound 1 (free base) was observed in all the solvent systems assessed. Following Salt Preparation Method 1 and using 1 mol. equiv. of benzoic acid, all the isolated materials were observed to be solids. Crystalline Form 1 Compound 1 benzoate was obtained from ethyl acetate and MEK samples. The sample from MEK became poorly crystalline after drying under vacuum at ca. 40° C., in which the crystallinity was improved upon 24 h storage at 40° C./75% RH; additional peaks corresponding to crystalline Form 3 Compound 1 benzoate were also observed in the sample. Crystalline Form 2 Compound 1 benzoate was obtained from ethanol and 2-propanol:water (75:25% v/v). Upon drying under vacuum at ca. 40° C., the sample from ethanol became amorphous while the sample from 2-propanol: water (75:25% v/v) retained its Form 2 Compound 1 benzoate crystallinity. Upon storage at 40° C./75% RH, both samples became crystalline mixtures of crystalline Form 2 Compound 1 benzoate and additional peaks, which corresponded to crystalline Form 3 Compound 1 benzoate. The damp sample from THE was found to be a mixture of crystalline Form 1 Compound 1 benzoate and Form 2 Compound 1 benzoate, which became amorphous upon drying under vacuum at ca. 40° C. Conversion to crystalline Form 3 Compound 1 benzoate was observed upon storage at 40° C./75% RH. The damp and dried solids from acetonitrile were found to be amorphous but conversion to mixtures of crystalline Form 1 Compound 1 benzoate, crystalline Form 2 Compound 1 benzoate and crystalline Form 3 Compound 1 benzoate were observed upon storage at 40° C./75% RH. The results are summarized in Table 80.

TABLE 80
Salt screen - Benzoic acid, 1 equiv.

| | | XRPD Analysis | | |
|---|---|---|---|---|
| Initial Solvent System | AS | Damp | Dry | 40° C. 75% RH |
| Acetonitrile | BME | A (evap.) | | 1 + 2 + 3 |
| Ethanol | Heptane | 2* | A | 2 + 3 |
| Ethyl acetate | Heptane | 1 | 1 | 1 |
| MEK | Heptane | 1* | 1, PC | 1 + 3 |
| THF | Heptane | 1 + 2 | A | 3 |
| IPA:H₂O (75:25% v/v) | tBME | 2 | 2 | 2 + 3 |

A = amorphous
PC = poorly crystalline
*= predominantly
1 = Form 1 Compound 1 benzoate
2 = Form 2 Compound 1 benzoate
3 = Form 3 Compound 1 benzoate

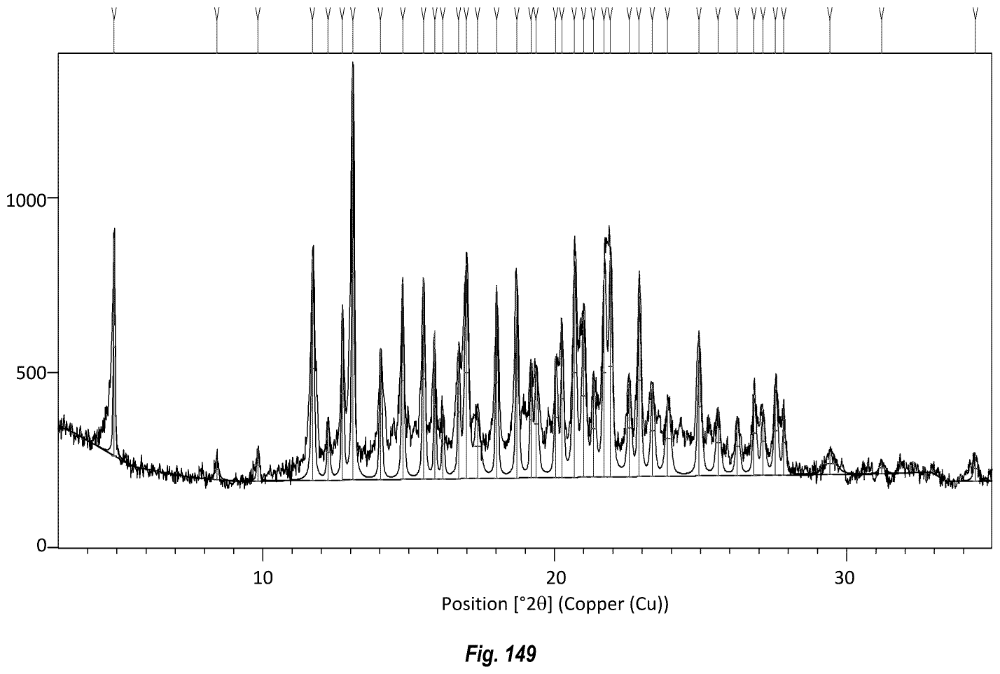

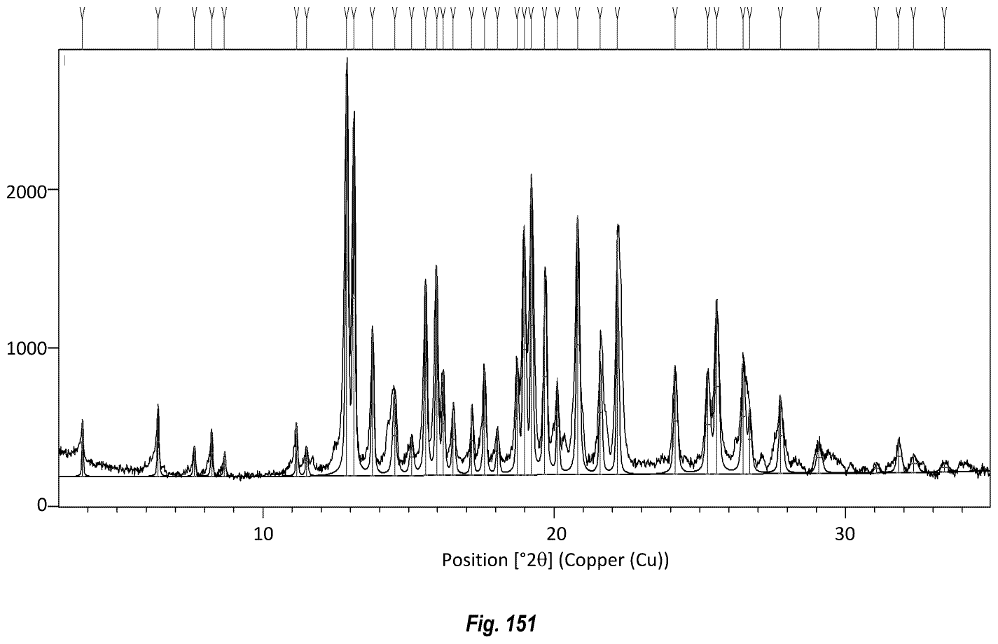

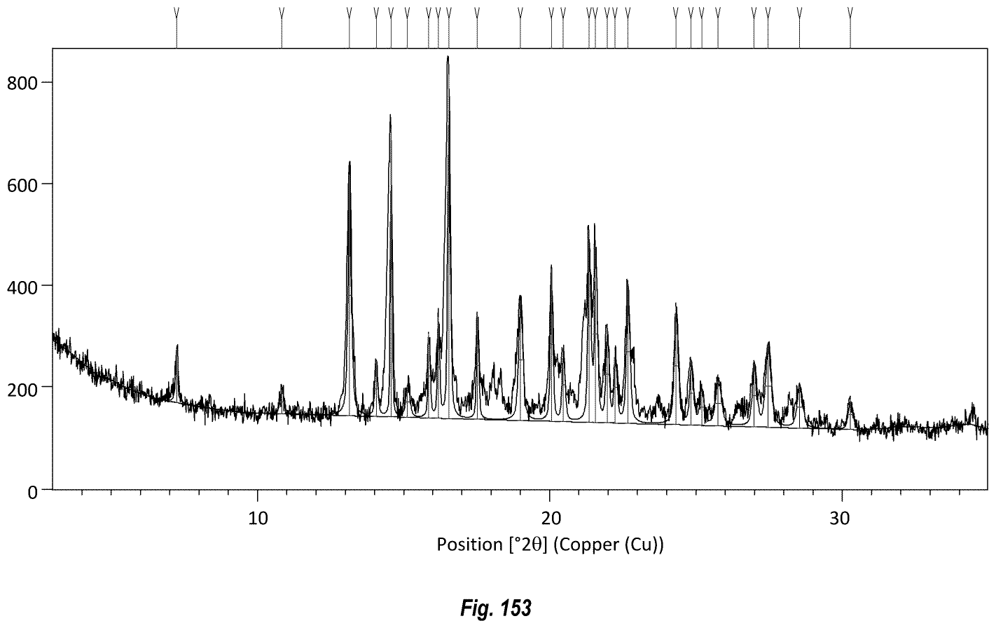

The XRPDs of crystalline Form 1 Compound 1 benzoate are shown in FIGS. 148 and 149. The XRPDs of crystalline Form 2 Compound 1 benzoate are shown in FIGS. 150 and 151. The XRPDs of crystalline Form 3 Compound 1 benzoate are shown in FIGS. 152 and 153. Crystalline Form 1 Compound 1 benzoate, crystalline Form 2 Compound 1 benzoate, and crystalline Form 3 Compound 1 benzoate obtained from ethyl acetate, 2-propanol:water (75:25% v/v), and THF, respectively, were further analyzed as discussed below.

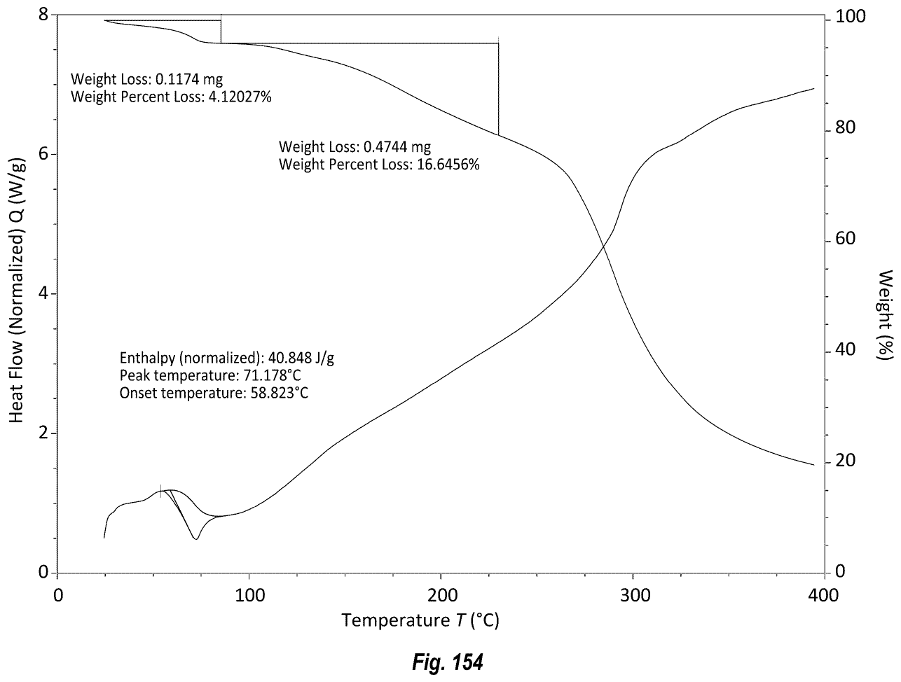

Crystalline Form 1 Compound 1 benzoate—The TG/DSC result of the dried sample from ethyl acetate showed 4.1% mass loss between the outset −85° C., followed by another mass loss of 16.6% between 85-230° C. (theoretically, 16.3%=1.0 equiv. of benzoic acid). See FIG. 154. An endothermic event was observed at peak onset 59° C. (peak at 71° C.).

The ¹H-NMR spectrum was consistent with the structure of the molecule, with peak shifting observed when compared with Compound 1 (an indication of salt formation). Peaks corresponding to 1.0 equiv. of the counterion (benzoic acid) were observed at δ 7.39, 7.47, and 7.96 ppm. Peaks corresponding to 1.7% w/w ethyl acetate and 3.0% w/w heptane were observed in the spectrum.

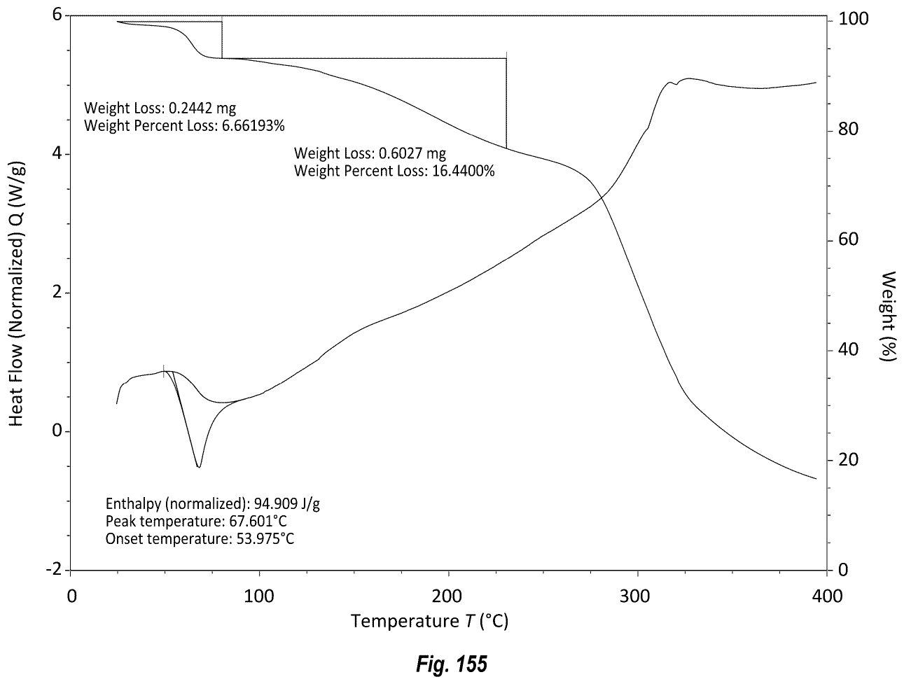

Crystalline Form 2 Compound 1 benzoate—The TG/DSC result of the dried sample from 2-propanol:water (75:25% v/v) showed 6.7% mass loss between the outset −80° C. (theoretically, 0.6 equiv. of 2-propanol or 1 equiv. of water), followed by another mass loss of 16.4% between 80-230° C. (theoretically, 16.3%=1.0 equiv. of benzoic acid). See FIG. 155. An endothermic event was observed at peak onset 54° C. (peak at 68° C.).

The ¹H-NMR spectrum was consistent with the structure of the molecule, with peak shifting observed when compared with input Compound 1 (an indication of salt formation). Peaks corresponding to 1.0 equiv. of the counterion (benzoic acid) were observed at δ 7.36, 7.43 and 7.96 ppm. Peaks corresponding to 4.3% w/w 2-propanol and 0.2% w/w heptane were observed in the spectrum.

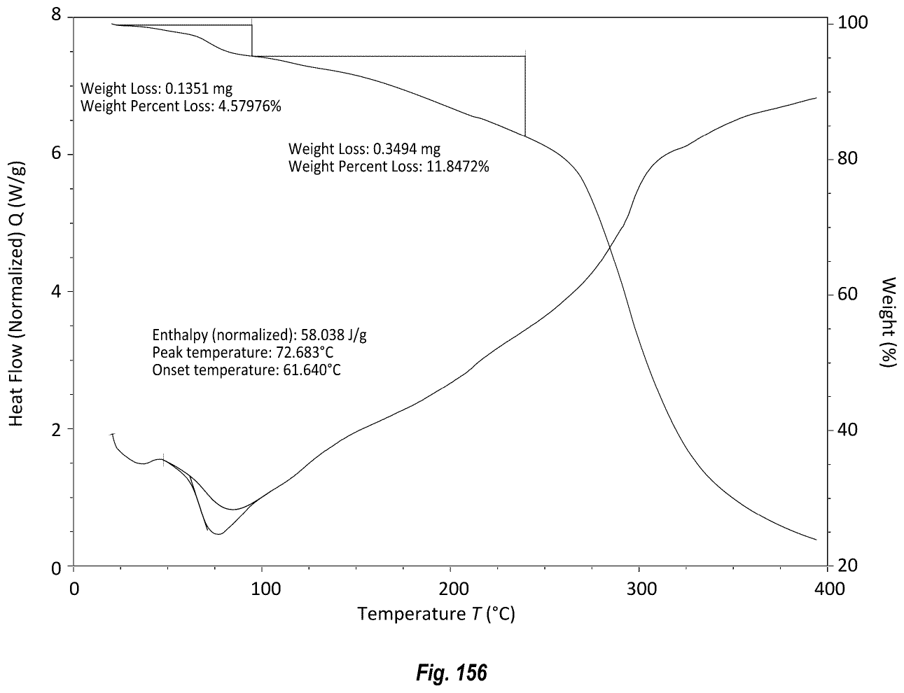

Crystalline Form 3 Compound 1 benzoate—The TG/DSC result of the 40° C./75% RH sample from THE showed 4.6% mass loss between the outset −95° C. (2 equiv. of water), followed by another mass loss of 11.8% between 95-240° C. (theoretically, 16.3%=1.0 equiv. of benzoic acid). See FIG. 156. An endothermic event was observed at peak onset 62° C. (peak at 73° C.).

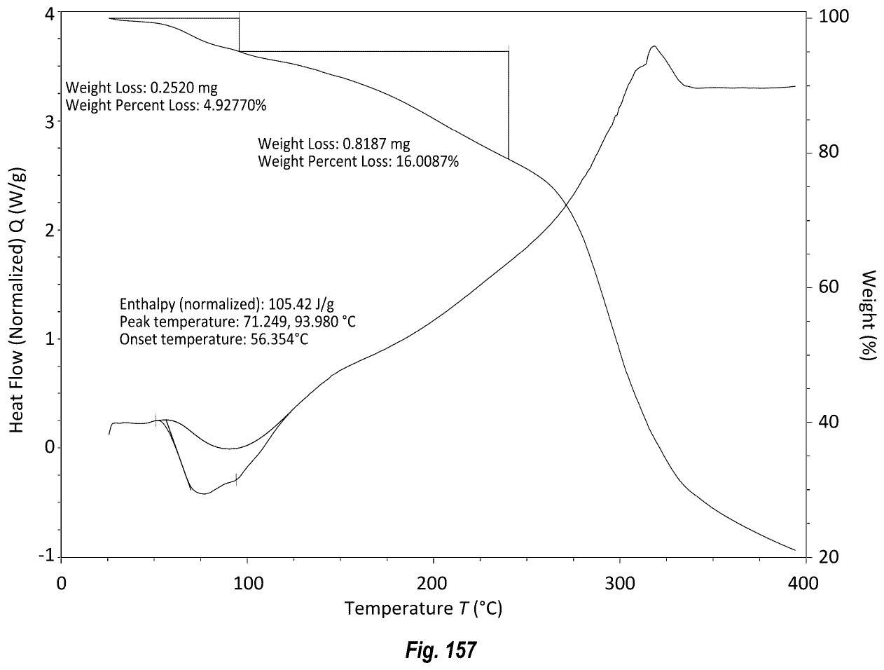

Mixture of Crystalline Form 1 Compound 1 benzoate, crystalline Form 2 Compound 1 benzoate, and crystalline Form 3 Compound 1 benzoate—The TG/DSC result of the 40° C./75% RH sample from acetonitrile showed 4.9% mass loss between the outset −95° C. (2.2 equiv. of water), followed by another mass loss of 16.0% between 95-240° C. (theoretically, 16.3%=1.0 equiv. of benzoic acid). See FIG. 157. An endothermic event was observed at peak onset 56° C. (peak at 71° C.).

### EQUIVALENTS

The foregoing written specification is considered to be sufficient to enable one skilled in the art to practice the embodiments. The foregoing description and Examples detail certain embodiments and describe the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. Unless otherwise set forth herein including, but not limited to, peak positions in two-theta of XRPD peaks, the term “about” generally refers to a range of numerical values (e.g., +/−5-10% of the recited range) that one of ordinary skill in the art would consider equivalent to the recited value (e.g., having the same function or result). When terms such as “at least” and “about” precede a list of numerical values or ranges, the terms modify all of the values or ranges provided in the list. In some instances, the term about may include numerical values that are rounded to the nearest significant figure.

1-271. (canceled)

272. An inorganic salt of Compound 1 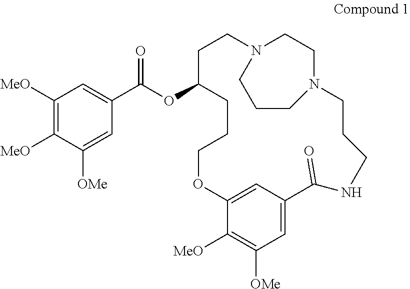 [SMILES: COc1cc(C(=O)O[C@@H]2CCCOc3cc(cc(OC)c3OC)C(=O)NCCCN3CCCN(CC2)CC3)cc(OC)c1OC]
or a hydrate or solvate thereof, wherein the salt or hydrate or solvate thereof is not a hexafluorophosphate salt of Compound 1 or a hydrate or solvate thereof.

273. An organic salt of Compound 1 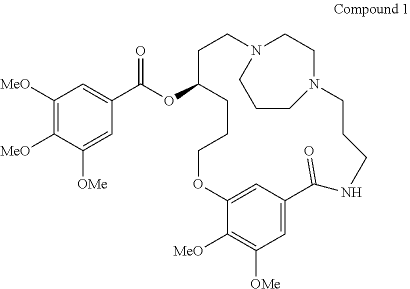 [SMILES: COc1cc(C(=O)O[C@@H]2CCCOc3cc(cc(OC)c3OC)C(=O)NCCCN3CCCN(CC2)CC3)cc(OC)c1OC]
or a hydrate or solvate thereof.

274. A crystalline salt comprising Compound 1 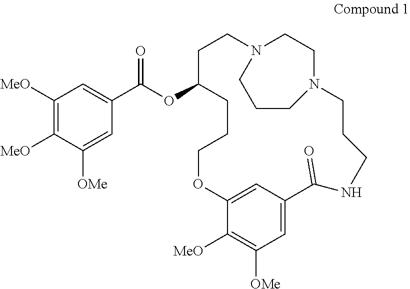 [SMILES: COc1cc(C(=O)O[C@@H]2CCCOc3cc(cc(OC)c3OC)C(=O)NCCCN3CCCN(CC2)CC3)cc(OC)c1OC]
and a salt coformer, or a hydrate or solvate thereof.

275. The crystalline salt or hydrate or solvate thereof of claim 274, wherein the salt coformer is an acid.

276. An amorphous salt comprising Compound 1 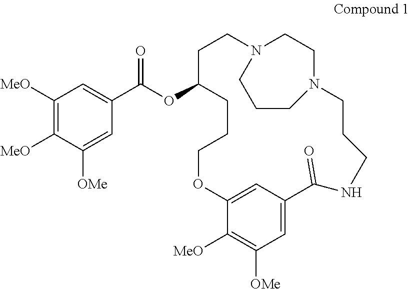 [SMILES: COc1cc(C(=O)O[C@@H]2CCCOc3cc(cc(OC)c3OC)C(=O)NCCCN3CCCN(CC2)CC3)cc(OC)c1OC]
and a salt coformer, or a hydrate or solvate thereof.

277. The amorphous salt or hydrate or solvate thereof of claim 276, wherein the salt coformer is an acid.

278. Compound 1 hydrogen sulfate or a hydrate or solvate thereof.

279. The Compound 1 hydrogen sulfate or hydrate or solvate thereof of claim 278, wherein the hydrogen sulfate is a mono(hydrogen sulfate) or a hydrate or solvate thereof.

280. The Compound 1 hydrogen sulfate or hydrate or solvate thereof of claim 278, wherein the hydrogen sulfate is a di(hydrogen sulfate) or a hydrate or solvate thereof.

281. The Compound 1 hydrogen sulfate or hydrate or solvate thereof of claim 280, wherein the di(hydrogen sulfate) is hydrate.

282. The Compound 1 hydrogen sulfate or hydrate or solvate thereof of claim 278, wherein the hydrogen sulfate is crystalline.

283. The Compound 1 hydrogen sulfate or hydrate or solvate thereof of claim 282, wherein the hydrogen sulfate is selected from crystalline Form 1 Compound 1 di(hydrogen sulfate), crystalline Form 3 Compound 1 di(hydrogen sulfate), crystalline Form 4 Compound 1 mono(hydrogen sulfate), crystalline Form 5 Compound 1 di(hydrogen sulfate), crystalline Form 6 Compound 1 di(hydrogen sulfate), crystalline Form 7 Compound 1 di(hydrogen sulfate), crystalline Form 8 Compound 1 mono(hydrogen sulfate), crystalline Form 9 Compound 1 di(hydrogen sulfate), crystalline Form 10 Compound 1 mono(hydrogen sulfate), and hydrates or solvates thereof.

284. The Compound 1 hydrogen sulfate or hydrate or solvate thereof of claim 282, wherein the hydrogen sulfate is crystalline Form 2 Compound 1 di(hydrogen sulfate) or a hydrate or solvate thereof.

285. Compound 1 hydrogen chloride or a hydrate or solvate thereof.

286. The Compound 1 hydrogen chloride or hydrate or solvate thereof of claim 285, wherein the hydrogen chloride is crystalline.

287. The Compound 1 hydrogen chloride or hydrate or solvate thereof of claim 286, wherein the hydrogen chloride is crystalline Form 1 Compound 1 hydrogen chloride or a hydrate or solvate thereof.

288. Compound 1 hydrogen phosphate or a hydrate or solvate thereof.

289. The Compound 1 hydrogen phosphate or hydrate or solvate thereof of claim 288, wherein the hydrogen phosphate is crystalline.

290. The Compound 1 hydrogen phosphate or hydrate or solvate thereof of claim 289, wherein the hydrogen phosphate is selected from crystalline Form 1 Compound 1 hydrogen phosphate, crystalline Form 2 Compound 1 hydrogen phosphate, crystalline Form 3 Compound 1 hydrogen phosphate, crystalline Form 4 Compound 1 hydrogen phosphate, crystalline Form 5 Compound 1 hydrogen phosphate, crystalline Form 6 Compound 1 hydrogen phosphate, crystalline Form 7 Compound 1 hydrogen phosphate, crystalline Form 8 Compound 1 hydrogen phosphate, and hydrates and solvates thereof.

291. Compound 1 fumarate or a hydrate or solvate thereof.

292. The Compound 1 fumarate or hydrate or solvate thereof of claim 291, wherein the fumaric acid is crystalline.

293. The Compound 1 fumarate or hydrate or solvate thereof of claim 292, wherein the fumarate is selected from crystalline Form 1 Compound 1 fumarate or a hydrate or solvate thereof, crystalline Form 2 Compound 1 fumarate or a hydrate or solvate thereof, crystalline Form 3 Compound 1 fumarate, and hydrates and solvates thereof.

294. Compound 1 malate or a hydrate or solvate thereof.

295. The Compound 1 malate or hydrate or solvate thereof of claim 294, wherein the malate is crystalline.

296. The Compound 1 malate or hydrate or solvate thereof of claim 295, wherein the malate is crystalline Form 1 Compound 1 malate or a hydrate or solvate thereof.

297. Compound 1 benzoate or a hydrate or solvate thereof.

298. The Compound 1 benzoate or hydrate or solvate thereof of claim 297, wherein the benzoate is crystalline.

299. The Compound 1 benzoate or hydrate or solvate thereof of claim 298, wherein the benzoate is selected from crystalline Form 1 Compound 1 benzoate, crystalline Form 2 Compound 1 benzoate, crystalline Form 3 Compound 1 benzoate, and hydrates and solvates thereof.

300. The amorphous salt or hydrate or solvate thereof of claim 276, wherein the salt is selected from Compound 1 p-tosylate, Compound 1 mesylate, Compound 1 benzenesulfonate, Compound 1 L-tartrate, Compound 1 citrate, Compound 1 maleate, and hydrates and solvates thereof.

301. Crystalline Compound 1 and hydrates and solvates thereof.

302. The crystalline Compound 1 or hydrate or solvate thereof of claim 301, wherein the crystalline Compound 1 is selected from crystalline Form 1 Compound 1, crystalline Form 1 Compound 2, and hydrates and solvates thereof.

303. A pharmaceutical composition comprising an inorganic salt of Compound 1 or a hydrate or solvate thereof of claim 272 and at least one pharmaceutically acceptable excipient.

304. A pharmaceutical composition comprising an organic salt of Compound 1 or a hydrate or solvate thereof of claim 273 and at least one pharmaceutically acceptable excipient.

305. A method of inhibiting ENT1 in a patient need thereof, comprising: administering to said patient an effective amount of an inorganic salt of Compound 1 or hydrate or solvate thereof of claim 272.

306. A method of inhibiting ENT1 in a patient need thereof, comprising: administering to said patient an effective amount of an organic salt of Compound 1 or hydrate or solvate thereof of claim 273.

307. A method of treating cancer in a patient need thereof, comprising: administering to said patient an effective amount of an inorganic salt of Compound 1 or hydrate or solvate thereof of claim 272.

308. A method of treating cancer in a patient need thereof, comprising: administering to said patient an effective amount of an organic salt of Compound 1 or hydrate or solvate thereof of claim 273.
